SEQUENCE LISTING (165 sequences)

SEQ ID NO: 1 - RNA, 1490 nt, unidentified
  source 1..1490: mol_type = rRNA; note = DP1 16S rRNA sequence
agtcagacatgcaagtcgagcggtagagagaagcttgcttctcttgagagcggcggacgggtgagtaaagcctaggaatctgcctggtagtgggggataacgttcggaaacggacgctaataccgcatacgtcctacgggagaaagcaggggaccttcgggccttgcgctatcagatgagcctaggtcggattagctagttggtgaggtaatggctcaccaaggcgacgatccgtaactggtctgagaggatgatcagtcacactggaactgagacacggtccagactcctacgggaggcagcagtggggaatattggacaatgggcgaaagcctgatccagccatgccgcgtgtgtgaagaaggtcttcggattgtaaagcactttaagttgggaggaagggcattaacctaatacgttagtgttttgacgttaccgacagaataagcaccggctaactctgtgccagcagccgcggtaatacagagggtgcaagcgttaatcggaattactgggcgtaaagcgcgcgtaggtggtttgttaagttggatgtgaaatccccgggctcaacctgggaactgcattcaaaactgactgactagagtatggtagagggtggtggaatttcctgtgtagcggtgaaatgcgtagatataggaaggaacaccagtggcgaaggcgaccacctggactaatactgacactgaggtgcgaaagcgtggggagcaaacaggattagataccctggtagtccacgccgtaaacgatgtcaactagccgttgggagccttgagctcttagtggcgcagctaacgcattaagttgaccgcctggggagtacggccgcaaggttaaaactcaaatgaattgacgggggcccgcacaagcggtggagcatgtggtttaattcgaagcaacgcgaagaaccttaccaggccttgacatccaatgaactttctagagatagattggtgccttcgggaacattgagacaggtgctgcatggctgtcgtcagctcgtgtcgtgagatgttgggttaagtcccgtaacgagcgcaacccttgtccttagttaccagcacgtaatggtgggcactctaaggagactgccggtgacaaaccggaggaaggtggggatgacgtcaagtcatcatggcccttacggcctgggctacacacgtgctacaatggtcggtacagagggttgccaagccgcgaggtggagctaatcccataaaaccgatcgtagtccggatcgcagtctgcaactcgactgcgtgaagtcggaatcgctagtaatcgcgaatcagaatgtcgcggtgaatacgttcccgggccttgtacacaccgcccgtcacaccatgggagtgggttgcaccagaagtagctagtctaaccttcgggaggacggttaccacggtgtgattcatgactggggtgaagtcgtaacaaggtagccgtaggggaacctgcggctggatcacctcctt

SEQ ID NO: 2 - DNA, 1017 bp, unidentified
  source 1..1017: mol_type = genomic DNA; note = DP2 ITS sequence
ttgttgctcgagttcttgtttagatcttttacaataatgtgtatctttaatgaagatgngngcttaattgcgctgctttattagagtgtcgcagtagaagtagtcttgcttgaatctcagtcaacgtttacacacattggagtttttttactttaatttaattctttctgctttgaatcgaaaggttcaaggcaaaaaacaaacacaaacaattttattttattataattttttaaactaaaccaaaattcctaacggaaattttaaaataatttaaaactttcaacaacggatctcttggttctcgcatcgatgaaaaacgtaccgaattgcgataagtaatgtgaattgcaaatactcgtgaatcattgaatttttgaacgcacattgcgcccttgagcattctcaagggcatgcctgtttgagcgtcatttccttctcaaaaaataattttttattttttggttgtgggcgatactcagggttagcttgaaattggagactgtttcagtcttttttaattcaacacttancttctttggagacgctgttctcgctgtgatgtatttatggatttattcgttttactttacaagggaaatggtaatgtaccttaggcaaagggttgcttttaatattcatcaagtttgacctcaaatcaggtaggattacccgctgaacttaagcatatcaataagcggaggaaaagaaaccaactgggattaccttagtaacggcgagtgaagcggtaaaagctcaaatttgaaatctggtactttcagtgcccgagttgtaatttgtagaatttgtctttgattaggtccttgtctatgttccttggaacaggacgtcatagagggtgagantcccgtttgnngaggataccttttctctgtannactttttcnaagagtcgagttgnttgggaatgcagctcaaanngggtngnaaattccatctaaagctaaatattngncnagagaccganagcgacantacagngatggaaagangaaa

SEQ ID NO: 3 - RNA, 1548 nt, unidentified
  source 1..1548: mol_type = rRNA; note = DP3 16S rRNA sequence
attgagagtttgatcctggctcaggatgaacgctggcggcgtgcctaatacatgcaagtcgaacgcacagcgaaaggtgcttgcacctttcaagtgagtggcgaacgggtgagtaacacgtggacaacctgcctcaaggctggggataacatttggaaacagatgctaataccgaataaaactcagtgtcgcatgacacaaagttaaaaggcgctttggcgtcacctagagatggatccgcggtgcattagttagttggtggggtaaaggcctaccaagacaatgatgcatagccgagttgagagactgatcggccacattgggactgagacacggcccaaactcctacgggaggctgcagtagggaatcttccacaatgggcgaaagcctgatggagcaacgccgcgtgtgtgatgaaggctttcgggtcgtaaagcactgttgtacgggaagaacagctagaatagggaatgattttagtttgacggtaccataccagaaagggacggctaaatacgtgccagcagccgcggtaatacgtatgtcccgagcgttatccggatttattgggcgtaaagcgagcgcagacggttgattaagtctgatgtgaaagcccggagctcaactccggaatggcattggaaactggttaacttgagtgcagtagaggtaagtggaactccatgtgtagcggtggaatgcgtagatatatggaagaacaccagtggcgaaggcggcttactggactgtaactgacgttgaggctcgaaagtgtgggtagcaaacaggattagataccctggtagtccacaccgtaaacgatgaacactaggtgttaggaggtttccgcctcttagtgccgaagctaacgcattaagtgttccgcctggggagtacgaccgcaaggttgaaactcaaaggaattgacggggacccgcacaagcggtggagcatgtggtttaattcgaagcaacgcgaagaaccttaccaggtcttgacatcctttgaagcttttagagatagaagtgttctcttcggagacaaagtgacaggtggtgcatggtcgtcgtcagctcgtgtcgtgagatgttgggttaagtcccgcaacgagcgcaacccttattgttagttgccagcattcagatgggcactctagcgagactgccggtgacaaaccggaggaaggcggggacgacgtcagatcatcatgccccttatgacctgggctacacacgtgctacaatggcgtatacaacgagttgccaacccgcgagggtgagctaatctcttaaagtacgtctcagttcggattgtagtctgcaactcgactacatgaagtcggaatcgctagtaatcgcggatcagcacgccgcggtgaatacgttcccgggtcttgtacacaccgcccgtcacaccatgggagtttgtaatgcccaaagccggtggcctaaccttttaggaaggagccgtctaaggcaggacagatgactggggtgaagtcgtaacaaggtagccgtaggagaacctgcggctggatcacctccttt

SEQ ID NO: 4 - RNA, 1538 nt, unidentified
  source 1..1538: mol_type = rRNA; note = DP4 16S rRNA sequence
ttgaagagtttgatcatggctcagattgaacgctggcggcaggcctaacacatgcaagtcgagcggcagcggaaagtagcttgctactttgccggcgagcggcggacgggtgagtaatgtctgggaaactgcctgatggagggggataactactggaaacggtagctaataccgcatgacctcgaaagagcaaagtgggggatcttcggacctcacgccatcggatgtgcccagatgggattagctagtaggtgaggtaatggctcacctaggcgacgatccctagctggtctgagaggatgaccagccacactggaactgagacacggtccagactcctacgggaggcagcagtggggaatattgcacaatgggcgcaagcctgatgcagccatgccgcgtgtgtgaagaaggccttagggttgtaaagcactttcagcgaggaggaaggcatcatacttaatacgtgtggtgattgacgttactcgcagaagaagcaccggctaactccgtgccagcagccgcggtaatacggagggtgcaagcgttaatcggaattactgggcgtaaagcgcacgcaggcggtttgttaagtcagatgtgaaatccccgcgcttaacgtgggaactgcatttgaaactggcaagctagagtcttgtagaggggggtagaattccaggtgtagcggtgaaatgcgtagagatctggaggaataccggtggcgaaggcggccccctggacaaagactgacgctcaggtgcgaaagcgtggggagcaaacaggattagataccctggtagtccacgccgtaaacgatgtcgacttggaggttgttcccttgaggagtggcttccggagctaacgcgttaagtcgaccgcctggggagtacggccgcaaggttaaaactcaaatgaattgacgggggcccgcacaagcggtggagcatgtggtttaattcgatgcaacgcgaagaaccttacctactcttgacatccacggaatttggcagagatgccttagtgccttcgggaaccgtgagacaggtgctgcatggctgtcgtcagctcgtgttgtgaaatgttgggttaagtcccgcaacgagcgcaacccttatcctttgttgccagcgattcggtcgggaactcaaaggagactgccggtgataaaccggaggaaggtggggatgacgtcaagtcatcatggcccttacgagtagggctacacacgtgctacaatggcgcatacaaagagaagcgacctcgcgagagcaagcggacctcacaaagtgcgtcgtagtccggatcggagtctgcaactcgactccgtgaagtcggaatcgctagtaatcgtggatcagaatgccacggtgaatacgttcccgggccttgtacacaccgcccgtcacaccatgggagtgggttgcaaaagaagtaggtagcttaaccttcgggagggcgcttaccactttgtgattcatgactggggtgaagtcgtaacaaggtaaccgtaggggaacctgcggttggatcacctcctt

SEQ ID NO: 5 - DNA, 1042 bp, unidentified
  source 1..1042: mol_type = genomic DNA; note = DP5 ITS sequence
gcgcttattgcgcggcgaaaaaaccttacacacagtgttttttgttattacannaacttttgctttggtctggactagaaatagtttgggccagaggttactaaactaaacttcaatatttatattgaattgttatttatttaattgtcaatttgttgattaaattcaaaaaatcttcaaaactttcaacaacggatctcttggttctcgcatcgatgaagaacgcagcgaaatgcgataagtaatatgaattgcagattttcgtgaatcatcgaatctttgaacgcacattgcgccctctggtattccagagggcatgcctgtttgagcgtcatttctctctcaaaccttcgggtttggtattgagtgatactcttagtcgaactaggcgtttgcttgaaatgtattggcatgagtggtactggatagtgctatatgactttcaatgtattaggtttatccaactcgttgaatagtttaatggtatatttctcggtattctaggctcggccttacaatataacaaacaagtttgacctcaaatcaggtaggattacccgctgaacttaagcatatcaataagcggaggaaaagaaaccaacagggattgccttagtaacggcgagtgaagcggcaaaagctcaaatttgaaatctggcaccttcggtgtccgagttgtaatttgaagaaggtaactttggagttggctcttgtctatgttccttggaacaggacgtcacagagggtgagaatcccgtgcgatgagatgcccaattctatgtaaagtgctttcgaagagtcgagttgtttgggaatgcagctctaagtgggtggtaaattccatctaaagctaaatattggcgagagaccgatagcgaacaagtacagtgatggaaagatgaaaagaactttgaaaagagagtgaaaaagtacgtgaaattgttgaaagggaaagggcttgagatcagacttggtattttgcgatcctttccttcttggttgggttcctcgcagcttactgggncagcatcggtttggatgg

SEQ ID NO: 6 - RNA, 1165 nt, unidentified
  source 1..1165: mol_type = rRNA; note = DP6 16S rRNA sequence
gaaaggcggcttcggctgtcacttatggatggacccgcgtcgcattagctagttggtgaggtaacggctcaccaaggcaacgatgcgtagccgacctgagagggtgatcggccacactgggactgagacacggcccagactcctacgggaggcagcagtagggaatcttccgcaatggacgaaagtctgacggagcaacgccgcgtgagtgatgaaggctttcgggtcgtaaaactctgttgttagggaagaacaagtgctagttgaataagctgcaccttgacggtacctaaccagaaagccacggctaactacgtgccagcagccgcggtaatacgtaggtggcaagcgttatccggaattattgggcgtaaagcgcgcgcaggtggtttcttaagtctgatgtgaaagcccacggctcaaccgtggagggtcattggaaactgggagacttgagtgcagaagaggaaagtggaattccatgtgtagcggtgaaatgcgtagagatatggaggaacaccagtggcgaaggcgactttctggtctgtaactgacactgaggcgcgaaagcgtggggagcaaacaggattagataccctggtagtccacgccgtaaacgatgagtgctaagtgttagagggtttccgccctttagtgctgaagttaacgcattaagcactccgcctggggagtacggccgcaaggctgaaactcaaaggaattgacgggggcccgcacaagcggtggagcatgtggtttaattcgaagcaacgcgaagaaccttaccaggtcttgacatcctctgaaaaccctagagatagggcttctccttcgggagcagagtgacaggtggtgcatggttgtcgtcagctcgtgtcgtgagatgttgggttaagtcccgcaacgagcgcaacccttgatcttagttgccatcattaagttgggcactctaaggtgactgccggtgacaaaccggaggaaggtggggatgacgtcaaatcatcatgccccttatgacctgggctacacacgtgctacaatggacggtacaaagagctgcaagaccgcgaggtggagctaatctcataaaaccgttctcagttcggattgtaggctgcaactcgcctacatgaagctggaatcgctagtaatcgcggatcagcat

SEQ ID NO: 7 - DNA, 871 bp, unidentified
  source 1..871: mol_type = genomic DNA; note = DP7 ITS sequence
ccacnctgcgtgggcgacacgaaacaccgaaaccgaacgcacgccgtcaagcaagaaatccacaaaactttcaacaacggatctcttggttctcgcatcgatgaagagcgcagcgaaatgcgatacctagtgtgaattgcagccatcgtgaatcatcgagttcttgaacgcacattgcgcccgctggtattccggcgggcatgcctgtctgagcgtcgtttccttcttggagcggagcttcagacctggcgggctgtctttcgggacggcgcgcccaaagcgaggggccttctgcgcgaactagactgtgcgcgcggggcggccggcgaacttataccaagctcgacctcagatcaggcaggagtacccgctgaacttaagcatatcaataagcggaggaaaagaaaccaacagggattgccccagtagcggcgagtgaagcggcaaaagctcagatttggaatcgcttcggcgagttgtgaattgcaggttggcgcctctgcggcggcggcggtccaagtcccttggaacagggcgccattgagggtgagagccccgtgggaccgtttgcctatgctctgaggcccttctgacgagtcgagttgtttgggaatgcagctctaagcgggtggtaaattccatctaaggctaaatactggcgagagaccgatagcgaacaagtactgtgaaggaaagatgaaaagcactttgaaaagagagtgaaacagcacgtgaaattgttgaaagggaagggtattgcgcccgacatggagcgtgcgcaccgctgcccctcgtgggcggcgctctgggcgtgctctgggccagcatcggtttttgccgcgggagaagggcggcgggcatgtagctcttc

SEQ ID NO: 8 - DNA, 942 bp, unidentified
  source 1..942: mol_type = genomic DNA; note = DP8 ITS sequence
gttgctcgagttcttgtttagatcttttacnataatgtgtatctttaatgaagatgtgcgcttaattgcgctgctttattagagtgtcgcagtagaagtagtcttgcttgaatctcagtcaacgtttacacacattggagtttttttactttaatttaattctttctgctttgaatcgaaaggttcaaggcaaaaaacaaacacaaacaattttattttattataattttttaaactaaaccaaaattcctaacggaaattttaaaataatttaaaactttcaacaacggatctcttggttctcgcatcgatgaaaaacgtagcgaattgcgataagtaatgtgaattgcaaatactcgtgaatcattgaatttttgaacgcacattgcgcccttgagcattctcaagggcatgcctgtttgagcgtcatttccttctcaaaagataattttttattttttggttgtgggcgatactcagggttagcttgaaattggagactgtttcagtcttttttaattcaacacttancttctttggagacgctgttctcgctgtgatgtatttatggatttattcgttttactttacaagggaaatggtaatgtaccttaggcaaagggttgcttttaatattcatcaagtttgacctcaaatcaggtaggattacccgctgaacttaagcatatcaataagcggaggaaaagaaaccaactgggattaccttagtaacggcgagtgaagcggtaaaagctcaaatttgaaatctggtactttcanngcccgagttgtaatttgtagaatttgtctttgattaggtccttgtctatgttccttggancaggacgtcatanagggtgantcccntttggcganganaccttttctctgtanactttttcnanagtcgagttgtttnggatgcagctcnaagtggggngg

SEQ ID NO: 9 - RNA, 1572 nt, unidentified
  source 1..1572: mol_type = rRNA; note = DP9 16S rRNA sequence
atgagagtttgatcttggctcaggatgaacgctggcggcgtgcctaatacatgcaagtcgaacgaacttccgttaattgattatgacgtacttgtactgattgagattttaacacgaagtgagtggcgaacgggtgagtaacacgtgggtaacctgcccagaagtaggggataacacctggaaacagatgctaataccgtataacagagaaaaccgcatggttttcttttaaaagatggctctgctatcacttctggatggacccgcggcgtattagctagttggtgaggcaaaggctcaccaaggcagtgatacgtagccgacctgagagggtaatcggccacattgggactgagacacggcccagactcctacgggaggcagcagtagggaatcttccacaatggacgcaagtctgatggagcaacgccgcgtgagtgaagaagggtttcggctcgtaaagctctgttgttaaagaagaacgtgggtaagagtaactgtttacccagtgacggtatttaaccagaaagccacggctaactacgtgccagcagccgcggtaatacgtaggtggcaagcgttatccggatttattgggcgtaaagcgagcgcaggcggtcttttaagtctaatgtgaaagccttcggctcaaccgaagaagtgcattggaaactgggagacttgagtgcagaagaggacagtggaactccatgtgtagcggtgaaatgcgtagatatatggaagaacaccagtggcgaaggcggctgtctggtctgcaactgacgctgaggctcgaaagcatgggtagcgaacaggattagataccctggtagtccatgccgtaaacgatgattactaagtgttggagggtttccgcccttcagtgctgcagctaacgcattaagtaatccgcctggggagtacgaccgcaaggttgaaactcaaaagaattgacgggggcccgcacaagcggtggagcatgtggtttaattcgaagctacgcgaagaaccttaccaggtcttgacatcttctgacagtctaagagattagaggttcccttcggggacagaatgacaggtggtgcatggttgtcgtcagctcgtgtcgtgagatgttgggttaagtcccgcaacgagcgcaacccttattactagttgccagcattaagttgggcactctagtgagactgccggtgacaaaccggaggaaggtggggacgacgtcaaatcatcatgccccttatgacctgggctacacacgtgctacaatggatggtacaacgagtcgcgagaccgcgaggttaagctaatctcttaaaaccattctcagttcggactgtaggctgcaactcgcctacacgaagtcggaatcgctagtaatcgcggatcagcatgccgcggtgaatacgttcccgggccttgtacacaccgcccgtcacaccatgagagtttgtaacacccaaagccggtggggtaaccttttaggagctagccgtctaaggtgggacagatgattagggtgaagtcgtaacaaggtagccgtaggagaacctgcggctggatcacctcctt

SEQ ID NO: 10 - RNA, 1300 nt, unidentified
  source 1..1300: mol_type = rRNA; note = DP10 16S rRNA sequence
cagatagttggtgaggtaacggctcaccaaggcaacgatgcgtagccgacctgagagggtgatcggccacactgggactgagacacggcccagactcctacgggaggcagcagtagggaatcttccgcaatggacgaaagtctgacggagcaacgccgcgtgagtgatgaaggttttcggatcgtaaagctctgttgttagggaagaacaagtgccgttcaaatagggcggcaccttgacggtacctaaccagaaagccacggctaactacgtgccagcagccgcggtaatacgtaggtggcaagcgttgtccggaattattgggcgtaaagggctcgcaggcggtttcttaagtctgatgtgaaagcccccggctcaaccggggagggtcattggaaactggggaacttgagtgcagaagaggagagtggaattccacgtgtagcggtgaaatgcgtagagatgtggaggaacaccagtggcgaaggcgactctctggtctgtaactgacgctgaggagcgaaagcgtggggagcgaacaggattagataccctggtagtccacgccgtaaacgatgagtgctaagtgttagggggtttccgccccttagtgctgcagctaacgcattaagcactccgcctggggagtacggtcgcaagactgaaactcaaaggaattgacgggggcccgcacaagcggtggagcatgtggtttaattcgaagcaacgcgaagaaccttaccaggtcttgacatcctctgacaatcctagagataggacgtccccttcgggggcagagtgacaggtggtgcatggttgtcgtcagctcgtgtcgtgagatgttgggttaagtcccgcaacgagcgcaacccttgatcttagttgccagcattcagttgggcactctaaggtgactgccggtgacaaaccggaggaaggtggggatgacgtcaaatcatcatgccccttatgacctgggctacacacgtgctacaatggacagaacaaagggcagcgaaaccgcgaggttaagccaatcccacaaatctgttctcagttcggatcgcagtctgcaactcgactgcgtgaagctggaatcgctagtaatcgcggatcagcatgccgcggtgaatacgttcccgggccttgtacacaccgcccgtcacaccacgagagtttgtaacacccgaagtcggtgaggtaaccttttaggagccagccgccgaaggtgggacagatgattggggtgaagtcgtaacaaggtagccgtatcggaaggtgcggctggatcacctccttt

SEQ ID NO: 11 - RNA, 1419 nt, unidentified
  source 1..1419: mol_type = rRNA; note = DP11 16S rRNA sequence
tgaagagtttgatcatggctcagattgaacgctggcggcaggcctaacacatgcaagtcgagcggtagagagaagcttgcttctcttgagagcggcggacgggtgagtaatgcctaggaatctgcctggtagtgggggataacgttcggaaacggacgctaataccgcatacgtcctacgggagaaagcaggggaccttcgggccttgcgctatcagatgagcctaggtcggattagctagttggtgaggtaatggctcaccaaggcgacgatccgtaactggtctgagaggatgatcagtcacactggaactgagacacggtccagactcctacgggaggcagcagtggggaatattggacaatgggcgaaagcctgatccagccatgccgcgtgtgtgaagaaggtcttcggattgtaaagcactttaagttgggaggaagggttgtagattaatactctgcaattttgacgttaccgacagaataagcaccggctaactctgtgccagcagccgcggtaatacagagggtgcaagcgttaatcggaattactgggcgtaaagcgcgcgtaggtggttcgttaagttggatgtgaaagccccgggctcaacctgggaactgcattcaaaactgacgagctagagtatggtagagggtggtggaatttcctgtgtagcggtgaaatgcgtagatataggaaggaacaccagtggcgaaggcgaccacctggactgatactgacactgaggtgcgaaagcgtggggagcaaacaggattagataccctggtagtccacgccgtaaacgatgtcaactagccgttggaatccttgagattttagtggcgcagctaacgcattaagttgaccgcctggggagtacggccgcaaggttaaaactcaaatgaattgacgggggcccgcacaagcggtggagcatgtggtttaattcgaagcaacgcgaagaaccttaccaggccttgacatccaatgaactttccagagatggatgggtgccttcgggaacattgagacaggtgctgcatggctgtcgtcagctcgtgtcgtgagatgttgggttaagtcccgtaacgagcgcaacccttgtccttagttaccagcacgttatggtgggcactctaaggagactgccggtgacaaaccggaggaaggtggggatgacgtcaagtcatcatggcccttacggcctgggctacacacgtgctacaatggtcggtacaaagggttgccaagccgcgaggtggagctaatcccataaaaccgatcgtagtccggatcgcagtctgcaactcgactgcgtgaagtcggaatcgctagtaatcgcgaatcagaatgtcgcggtgaatacgttcccgggccttgtacacaccgcccgtcacatcccacacgaattgcttg

SEQ ID NO: 12 - RNA, 1520 nt, unidentified
  source 1..1520: mol_type = rRNA; note = DP12 16S rRNA sequence
tacggagagtttgatcctggctcaggatgaacgctggcggcgtgcttaacacatgcaagtcgaacggtgaagccaagcttgcttggtggatcagtggcgaacgggtgagtaacacgtgagcaacctgccctggactctgggataagcgctggaaacggcgtctaatactggatatgagccttcatcgcatggtgggggttggaaagattttttggtctgggatgggctcgcggcctatcagcttgttggtgaggtaatggctcaccaaggcgtcgacgggtagccggcctgagagggtgaccggccacactgggactgagacacggcccagactcctacgggaggcagcagtggggaatattgcacaatgggcgaaagcctgatgcagcaacgccgcgtgagggatgacggccttcgggttgtaaacctcttttagcagggaagaagcgaaagtgacggtacctgcagaaaaagcgccggctaactacgtgccagcagccgcggtaatacgtagggcgcaagcgttatccggaattattgggcgtaaagagctcgtaggcggtttgtcgcgtctgctgtgaaatcccgaggctcaacctcgggcctgcagtgggtacgggcagactagagtgcggtaggggagattggaattcctggtgtagcggtggaatgcgcagatatcaggaggaacaccgatggcgaaggcagatctctgggccgtaactgacgctgaggagcgaaagggtggggagcaaacaggcttagataccctggtagtccaccccgtaaacgttgggaactagttgtggggaccattccacggtttccgtgacgcagctaacgcattaagttccccgcctggggagtacggccgcaaggctaaaactcaaaggaattgacggggacccgcacaagcggcggagcatgcggattaattcgatgcaacgcgaagaaccttaccaaggcttgacatacaccagaacgggccagaaatggtcaactctttggacactggtgaacaggtggtgcatggttgtcgtcagctcgtgtcgtgagatgttgggttaagtcccgcaacgagcgcaaccctcgttctatgttgccagcacgtaatggtgggaactcatgggatactgccggggtcaactcggaggaaggtggggatgacgtcaaatcatcatgccccttatgtcttgggcttcacgcatgctacaatggccggtacaaagggctgcaataccgtgaggtggagcgaatcccaaaaagccggtcccagttcggattgaggtctgcaactcgacctcatgaagtcggagtcgctagtaatcgcagatcagcaacgctgcggtgaatacgttcccgggtcttgtacacaccgcccgtcaagtcatgaaagtcggtaacacctgaagccggtggcccaacccttgtggagggagccgtcgaaggtgggatcggtaattaggactaagtcgtaacaaggtagccgtaccggaaggtgcggctggatcacctccttt

SEQ ID NO: 13 - RNA, 1457 nt, unidentified
  source 1..1457: mol_type = rRNA; note = DP13 16S rRNA sequence
agttagcggcggacgggtgagtaacacgtgggtaacctgcctataagactgggataactccgggaaaccggggctaataccggataacattttgcaccgcatggtgcgaaattgaaaggcggcttcggctgtcacttatagatggacctgcggcgcattagctagttggtgaggtaacggctcaccaaggcgacgatgcgtagccgacctgagagggtgatcggccacactgggactgagacacggcccagactcctacgggaggcagcagtagggaatcttccgcaatggacgaaagtctgacggagcaacgccgcgtgaacgatgaaggctttcgggtcgtaaagttctgttgttagggaagaacaagtgctagttgaataagctggcaccttgacggtacctaaccagaaagccacggctaactacgtgccagcagccgcggtaatacgtaggtggcaagcgttatccggaattattgggcgtaaagcgcgcgcaggtggtttcttaagtctgatgtgaaagcccacggctcaaccgtggagggtcattggaaactgggagacttgagtgcagaagaggaaagtggaattccatgtgtagcggtgaaatgcgtagagatatggaggaacaccagtggcgaaggcgactttctggtctgcaactgacactgaggcgcgaaagcgtggggagcaaacaggattagataccctggtagtccacgccgtaaacgatgagtgctaagtgttagagggtttccgccctttagtgctgaagttaacgcattaagcactccgcctggggagtacggccgcaaggctgaaactcaaaggaattgacgggggcccgcacaagcggtggagcatgtggtttaattcgaagcaacgcgaagaaccttaccaggtcttgacatcctctgaaaaccctagagatagggcttccccttcgggggcagagtgacaggtggtgcatggttgtcgtcagctcgtgtcgtgagatgttgggttaagtcccgcaacgagcgcaacccttgatcttagttgccatcattaagttgggcactctaaggtgactgccggtgacaaaccggaggaaggtggggatgacgtcaaatcatcatgccccttatgacctgggctacacacgtgctacaatggacggtacaaagagtcgcaagaccgcgaggtggagctaatctcataaaaccgttctcagttcggattgtaggctgcaactcgcctacatgaagctggaatcgctagtaatcgcggatcagcatgccgcggtgaatacgttcccgggccttgtacacaccgcccgtcacaccacgagagtttgtaacacccgaagtcggtggggtaaccttttggagccagccgcctaaggtgggacagatgattggggtgaagtcgtaacaaggtagccgtatcggaaggtgcggctggatcacctccttt

SEQ ID NO: 14 - RNA, 1526 nt, unidentified
  source 1..1526: mol_type = rRNA; note = DP1416S rRNA sequence
tacggagagtttgatcctggctcaggatgaacgctggcggcgtgcttaacacatgcaagtcgaacgatgacttctgtgcttgcacagaatgattagtggcgaacgggtgagtaacacgtgagtaacctgcccttaacttcgggataagcctgggaaaccgggtctaataccggatacgacctcctggcgcatgccatggtggtggaaagctttagcggttttggatggactcgcggcctatcagcttgttggttggggtaatggcccaccaaggcgacgacgggtagccggcctgagagggtgaccggccacactgggactgagacacggcccagactcctacgggaggcagcagtggggaatattgcacaatgggcgaaagcctgatgcagcgacgccgcgtgagggatgacggccttcgggttgtaaacctctttcagcagggaagaagcgaaagtgacggtacctgcagaagaagcgccggctaactacgtgccagcagccgcggtaatacgtagggcgcaagcgttatccggaattattgggcgtaaagagctcgtaggcggtttgtcgcgtctgctgtgaaagcccggggctcaaccccgggtctgcagtgggtacgggcagactagagtgcagtaggggagactggaattcctggtgtagcggtgaaatgcgcagatatcaggaggaacaccgatggcgaaggcaggtctctgggctgtaactgacgctgaggagcgaaagcatggggagcgaacaggattagataccctggtagtccatgccgtaaacgttgggcactaggtgtgggggacattccacgttttccgcgccgtagctaacgcattaagtgccccgcctggggagtacggccgcaaggctaaaactcaaaggaattgacgggggcccgcacaagcggcggagcatgcggattaattcgatgcaacgcgaagaaccttaccaaggcttgacatgaaccggtaagacctggaaacaggtcccccacttgtggccggtttacaggtggtgcatggttgtcgtcagctcgtgtcgtgagatgttgggttaagtcccgcaacgagcgcaaccctcgttctatgttgccagcgggttatgccggggactcataggagactgccggggtcaactcggaggaaggtggggacgacgtcaaatcatcatgccccttatgtcttgggcttcacgcatgctacaatggccggtacaaagggttgcgatactgtgaggtggagctaatcccaaaaagccggtctcagttcggattgaggtctgcaactcgacctcatgaagttggagtcgctagtaatcgcagatcagcaacgctgcggtgaatacgttcccgggccttgtacacaccgcccgtcaagtcacgaaagttggtaacacccgaagccggtggcctaaccccttgtgggagggagccgtcgaaggtgggaccggcgattgggactaagtcgtaacaaggtagccgtaccggaaggtgcggctggatcacctccttt

SEQ ID NO: 15 - RNA, 1520 nt, unidentified
  source 1..1520: mol_type = rRNA; note = DP15 16S rRNA sequence
tacggagagtttgatcctggctcaggacgaacgctggcggcgtgcttaacacatgcaagtcgaacgatgatcaggagcttgctcctgtgattagtggcgaacgggtgagtaacacgtgagtaacctgcccctgactctgggataagcgttggaaacgacgtctaatactggatatgatcactggccgcatggtctggtggtggaaagattttttggttggggatggactcgcggcctatcagcttgttggtgaggtaatggctcaccaaggcgacgacgggtagccggcctgagagggtgaccggccacactgggactgagacacggcccagactcctacgggaggcagcagtggggaatattgcacaatgggcgaaagcctgatgcagcaacgccgcgtgagggatgacggccttcgggttgtaaacctcttttagtagggaagaagcgaaagtgacggtacctgcagaaaaagcaccggctaactacgtgccagcagccgcggtaatacgtagggtgcaagcgttgtccggaattattgggcgtaaagagctcgtaggcggtttgtcgcgtctgctgtgaaatcccgaggctcaacctcgggcttgcagtgggtacgggcagactagagtgcggtaggggagattggaattcctggtgtagcggtggaatgcgcagatatcaggaggaacaccgatggcgaaggcagatctctgggccgtaactgacgctgaggagcgaaagcgtggggagcgaacaggattagataccctggtagtccacgccgtaaacgttgggcgctagatgtagggacctttccacggtttctgtgtcgtagctaacgcattaagcgccccgcctggggagtacggccgcaaggctaaaactcaaaggaattgacgggggcccgcacaagcggcggagcatgcggattaattcgatgcaacgcgaagaaccttaccaaggcttgacatacaccggaaacggccagagatggtcgcccccttgtggtcggtgtacaggtggtgcatggttgtcgtcagctcgtgtcgtgagatgttgggttaagtcccgcaacgagcgcaaccctcgttctatgttgccagcgcgttatggcggggactcataggagactgccggggtcaactcggaggaaggtggggatgacgtcaaatcatcatgccccttatgtcttgggcttcacgcatgctacaatggccggtacaaagggctgcgataccgtaaggtggagcgaatcccaaaaagccggtctcagttcggattgaggtctgcaactcgacctcatgaagtcggagtcgctagtaatcgcagatcagcaacgctgcggtgaatacgttcccgggccttgtacacaccgcccgtcaagtcatgaaagtcggtaacacccgaagccggtggcctaacccttgtggaaggagccgtcgaaggtgggatcggtgattaggactaagtcgtaacaaggtagccgtaccggaaggtgcggctggatcacctccttt

SEQ ID NO: 16 - RNA, 1368 nt, unidentified
  source 1..1368: mol_type = rRNA; note = DP16 16S rRNA sequence
gcacttcatcgtggtgcaccgtgaagggtctttgggcgttttacacatgcaagcaagtgttctataatttaggttatggaacagccaaatggtcagtacagctcagtcctaggcgatggactccgtaaaacggggacagactatcctttaataattaataggtttattatttcaataataatctctaggaagggatatacatatatccttattagtctaaaggttaataaaccgccttagtcaggactgagttctcaacagctacgggttaaaccccaggcaacgacgagtaggggatagtgatagctacaaccccgacactggccgcaagccagggtacttaagtacgcagcagtgaagaatcctcggcaatgcatcgcaattaccggtgacccaatataaaataatatcagggaggtagtaggtgtgaccgggtgacccaaagacgagtagtgacataagttattattcgcgtatgtcgaacatgatagtgacgtgttcaacatcaagccccgtccaacctctgtgccagcagtcgcggtaaaacaggaggggcagctcttatggtcatgaatgggcgtatagggcacgcagccagttagtaaaagcttgaatatttatttttttaaaaagaatgtttgagaggctatgagtttttataaagtgtacccacgacaccagacttagggctgagatcctatgaagtctgggggcggtcctttagggtgcattgtaaaaactgacggtaaggtgcgacagctgggataccgaagcggagtagagcccgcctagccccagccgtaaacgataggggccgttgttgactacggttttcaataaggctaacgcctgagcccctcgcctgtagggtatagccgcaaggccgacatattaacgatgagaccgctggtgagcaaacgggtgcggggcatgctgttcaatcagacagtacgctgacaaccttaccactccttgaatcttttagattatatttctaaaatgacaggtgctgcatggccgtcgtcagttcgtggtcgtgagtcgtccggttgagtccatgaacgaacgcagacccgtctgtatactcagtgaaaagaaatttagctgaactatacagttgtacttctataaaaggtacctgtacgggattatgacaggtcgtcatggcctttatggagtgggctacaggcgtgccacacgagccgttttaacgagttcctcatttttatgaataaggtctcttaatcacggctagtatacggatcgtaggctgtaactcgcctacgtgaagtcggagtcccgagtaatcgccgatcatcacgcggcggtgaatctacactctcactggggtactaaccgctcgtcacg

SEQ ID NO: 17 - RNA, 898 nt, unidentified
  source 1..898: mol_type = rRNA; note = DP17 16S rRNA sequence
gtgattgacgttactcgcagaagaagcaccggctaactccgtgccagcagccgcggtaatacggagggtgcaagcgttaatcggaattactgggcgtaaagcgcacgcaggcggtttgttaagtcagatgtgaaatccccgcgcttaacgtgggaactgcatttgaaactggcaagctagagtcttgtagaggggggtagaattccaggtgtagcggtgaaatgcgtagagatctggaggaataccggtggcgaaggcggccccctggacaaagactgacgctcaggtgcgaaagcgtggggagcaaacaggattagataccctggtagtccacgctgtaaacgatgtcgacttggaggttgtgcccttgaggcgtggcttccggagctaacgcgttaagtcgaccgcctggggagtacggccgcaaggttaaaactcaaatgaattgacgggggcccgcacaagcggtggagcatgtggtttaattcgatgcaacgcgaagaaccttacctactcttgacatccacggaattcgccagagatggcttagtgccttcgggaaccgtgagacaggtgctgcatggctgtcgtcagctcgtgttgtgaaatgttgggttaagtcccgcaacgagcgcaacccttatcctttgttgccagcacgtaatggtgggaactcaaaggagactgccggtgataaaccggaggaaggtggggatgacgtcaagtcatcatggcccttacgagtagggctacacacgtgctacaatggcatatacaaagagaagcgaactcgcgagagcaagcggacctcataaagtatgtcgtagtccggattggagtctgcaactcgactccatgaagtcggaatcgctagtaatcgtagatcagaatgctacgg

SEQ ID NO: 18 - RNA, 1532 nt, unidentified
  source 1..1532: mol_type = rRNA; note = DP18 16S rRNA sequence
tgaagagtttgatcatggctcagattgaacgctggcggcaggcctaacacatgcaagtcgagcggatgaaaggagcttgctcctggattcagcggcggacgggtgagtaatgcctaggaatctgcctggtagtgggggacaacgtttcgaaaggaacgctaataccgcatacgtcctacgggagaaagcaggggaccttcgggccttgcgctatcagatgagcctaggtcggattagctagttggtgaggtaatggctcaccaaggcgacgatccgtaactggtctgagaggatgatcagtcacactggaactgagacacggtccagactcctacgggaggcagcagtggggaatattggacaatgggcgaaagcctgatccagccatgccgcgtgtgtgaagaaggtcttcggattgtaaagcactttaagttgggaggaagggcagtaaattaatactttgctgttttgacgttaccgacagaataagcaccggctaactctgtgccagcagccgcggtaatacagagggtgcaagcgttaatcggaattactgggcgtaaagcgcgcgtaggtggtttgttaagttgaatgtgaaatccccgggctcaacctgggaactgcatccaaaactggcaagctagagtatggtagagggtggtggaatttcctgtgtagcggtgaaatgcgtagatataggaaggaacaccagtggcgaaggcgaccacctggactgatactgacactgaggtgcgaaagcgtggggagcaaacaggattagataccctggtagtccacgccgtaaacgatgtcaactagccgttgggagccttgagctcttagtggcgcagctaacgcattaagttgaccgcctggggagtacggccgcaaggttaaaactcaaatgaattgacgggggcccgcacaagcggtggagcatgtggtttaattcgaagcaacgcgaagaaccttaccaggccttgacatccaatgaactttccagagatggattggtgccttcgggaacattgagacaggtgctgcatggctgtcgtcagctcgtgtcgtgagatgttgggttaagtcccgtaacgagcgcaacccttgtccttagttaccagcacgttatggtgggcactctaaggagactgccggtgacaaaccggaggaaggtggggatgacgtcaagtcatcatggcccttacggcctgggctacacacgtgctacaatggtcggtacaaagggttgccaagccgcgaggtggagctaatcccataaaaccgatcgtagtccggatcgcagtctgcaactcgactgcgtgaagtcggaatcgctagtaatcgcgaatcagaatgtcgcggtgaatacgttcccgggccttgtacacaccgcccgtcacaccatgggagtgggttgcaccagaagtagctagtctaaccttcgggaggacggttaccacggtgtgattcatgactggggtgaagtcgtaacaaggtagccgtaggggaacctgcggctggatcacctcctt

SEQ ID NO: 19 - RNA, 1520 nt, unidentified
  source 1..1520: mol_type = rRNA; note = DP19 16S rRNA sequence
tacggagagtttgatcctggctcaggacgaacgctggcggcgtgcttaacacatgcaagtcgaacgatgatgcccagcttgctgggtggattagtggcgaacgggtgagtaacacgtgagtaacctgcccctgactctgggataagcgttggaaacgacgtctaatactggatacgactgccggccgcatggtctggtggtggaaagattttttggttggggatggactcgcggcctatcagcttgttggtgaggtaatggctcaccaaggcgacgacgggtagccggcctgagagggtgaccggccacactgggactgagacacggcccagactcctacgggaggcagcagtggggaatattgcacaatgggcgaaagcctgatgcagcaacgccgcgtgagggatgacggccttcgggttgtaaacctcttttagtagggaagaagcgaaagtgacggtacctgcagaaaaagcaccggctaactacgtgccagcagccgcggtaatacgtagggtgcaagcgttgtccggaattattgggcgtaaagagctcgtaggcggtttgtcgcgtctgctgtgaaatcccgaggctcaacctcgggcttgcagtgggtacgggcagactagagtgcggtaggggagattggaattcctggtgtagcggtggaatgcgcagatatcaggaggaacaccgatggcgaaggcagatctctgggccgtaactgacgctgaggagcgaaagcgtggggagcgaacaggattagataccctggtagtccacgccgtaaacgttgggcgctagatgtagggacctttccacggtttctgtgtcgtagctaacgcattaagcgccccgcctggggagtacggccgcaaggctaaaactcaaaggaattgacgggggcccgcacaagcggcggagcatgcggattaattcgatgcaacgcgaagaaccttaccaaggcttgacatacaccggaaacggccagagatggtcgcccccttgtggtcggtgtacaggtggtgcatggttgtcgtcagctcgtgtcgtgagatgttgggttaagtcccgcaacgagcgcaaccctcgttctatgttgccagcgcgttatggcggggactcataggagactgccggggtcaactcggaggaaggtggggatgacgtcaaatcatcatgccccttatgtcttgggcttcacgcatgctacaatggccggtacaaagggctgcgataccgtaaggtggagcgaatcccaaaaagccggtctcagttcggattgaggtctgcaactcgacctcatgaagtcggagtcgctagtaatcgcagatcagcaacgctgcggtgaatacgttcccgggccttgtacacaccgcccgtcaagtcatgaaagtcggtaacacccgaagccggtggcctaacccttgtggaaggagccgtcgaaggtgggatcggtgattaggactaagtcgtaacaaggtagccgtaccggaaggtgcggctggatcacctccttt

SEQ ID NO: 20 - RNA, 1543 nt, unidentified
  source 1..1543: mol_type = rRNA; note = DP20 16S rRNA sequence
tgaagagtttgatcctggctcagagtgaacgctggcggtaggcctaacacatgcaagtcgaacggcagcacagtaagagcttgctcttatgggtggcgagtggcggacgggtgaggaatacatcggaatctaccttttcgtgggggataacgtagggaaacttacgctaataccgcatacgaccttcgggtgaaagcaggggaccttcgggccttgcgcggatagatgagccgatgtcggattagctagttggcggggtaaaggcccaccaaggcgacgatccgtagctggtctgagaggatgatcagccacactggaactgagacacggtccagactcctacgggaggcagcagtggggaatattggacaatgggcgcaagcctgatccagccataccgcgtgggtgaagaaggccttcgggttgtaaagcccttttgttgggaaagaaaagcagtcggctaatacccggttgttctgacggtacccaaagaataagcaccggctaacttcgtgccagcagccgcggtaatacgaagggtgcaagcgttactcggaattactgggcgtaaagcgtgcgtaggtggttgtttaagtctgttgtgaaagccctgggctcaacctgggaattgcagtggatactgggcgactagagtgtggtagagggtagtggaattcccggtgtagcagtgaaatgcgtagagatcgggaggaacatccatggcgaaggcagctacctggaccaacactgacactgaggcacgaaagcgtggggagcaaacaggattagataccctggtagtccacgccctaaacgatgcgaactggatgttgggtgcaatttggcacgcagtatcgaagctaacgcgttaagttcgccgcctggggagtacggtcgcaagactgaaactcaaaggaattgacgggggcccgcacaagcggtggagtatgtggtttaattcgatgcaacgcgaagaaccttacctggtcttgacatgtcgagaactttccagagatggattggtgccttcgggaactcgaacacaggtgctgcatggctgtcgtcagctcgtgtcgtgagatgttgggttaagtcccgcaacgagcgcaacccttgtccttagttgccagcacgtaatggtgggaactctaaggagaccgccggtgacaaaccggaggaaggtggggatgacgtcaagtcatcatggcccttacgaccagggctacacacgtactacaatggtagggacagagggctgcaaacccgcgagggcaagccaatcccagaaaccctatctcagtccggattggagtctgcaactcgactccatgaagtcggaatcgctagtaatcgcagatcagcattgctgcggtgaatacgttcccgggccttgtacacaccgcccgtcacaccatgggagtttgttgcaccagaagcaggtagcttaaccttcgggagggcgcttgccacggtgtggccgatgactggggtgaagtcgtaacaaggtagccgtatcggaaggtgcggctggatcacctccttt

SEQ ID NO: 21 - skipped

SEQ ID NO: 22 - RNA, 1539 nt, unidentified
  source 1..1539: mol_type = rRNA; note = DP22 16S rRNA sequence
ttgaagagtttgatcatggctcagattgaacgctggcggcaggcctaacacatgcaagtcgagcggcagcgggaagtagcttgctactttgccggcgagcggcggacgggtgagtaatgtctgggaaactgcctgatggagggggataactactggaaacggtagctaataccgcatgacctcgcaagagcaaagtgggggaccttcgggcctcacgccatcggatgtgcccagatgggattagctagtaggtgaggtaatggctcacctaggcgacgatccctagctggtctgagaggatgaccagccacactggaactgagacacggtccagactcctacgggaggcagcagtggggaatattgcacaatgggcgcaagcctgatgcagccatgccgcgtgtgtgaagaaggccttagggttgtaaagcactttcagcgaggaggaagggttcagtgttaatagcactgaacattgacgttactcgcagaagaagcaccggctaactccgtgccagcagccgcggtaatacggagggtgcaagcgttaatcggaattactgggcgtaaagcgcacgcaggcggtttgttaagtcagatgtgaaatccccgagcttaacttgggaactgcatttgaaactggcaagctagagtcttgtagaggggggtagaattccaggtgtagcggtgaaatgcgtagagatctggaggaataccggtggcgaaggcggccccctggacaaagactgacgctcaggtgcgaaagcgtggggagcaaacaggattagataccctggtagtccacgctgtaaacgatgtcgacttggaggttgtgcccttgaggcgtggcttccggagctaacgcgttaagtcgaccgcctggggagtacggccgcaaggttaaaactcaaatgaattgacgggggcccgcacaagcggtggagcatgtggtttaattcgatgcaacgcgaagaaccttacctactcttgacatccagagaattcgctagagatagcttagtgccttcgggaactctgagacaggtgctgcatggctgtcgtcagctcgtgttgtgaaatgttgggttaagtcccgcaacgagcgcaacccttatcctttgttgccagcgagtaatgtcgggaactcaaaggagactgccggtgataaaccggaggaaggtggggatgacgtcaagtcatcatggcccttacgagtagggctacacacgtgctacaatggcatatacaaagagaagcaaactcgcgagagcaagcggacctcataaagtatgtcgtagtccggattggagtctgcaactcgactccatgaagtcggaatcgctagtaatcgtagatcagaatgctacggtgaatacgttcccgggccttgtacacaccgcccgtcacaccatgggagtgggttgcaaaagaagtaggtagcttaaccttcgggagggcgcttaccactttgtgattcatgactggggtgaagtcgtaacaaggtaaccgtaggggaacctgcggttggatcacctcctt

SEQ ID NO: 23 - RNA, 1536 nt, unidentified
  source 1..1536: mol_type = rRNA; note = DP23 16S rRNA sequence
ttgaagagtttgatcatggctcagattgaacgctggcggcaggcctaacacatgcaagtcgaacggtagcacagagagcttgctcttgggtgacgagtggcggacgggtgagtaatgtctgggaaactgcccgatggagggggataactactggaaacggtagctaataccgcataacgtcttcggaccaaagtgggggaccttcgggcctcacaccatcggatgtgcccagatgggattagctagtaggtggggtaatggctcacctaggcgacgatccctagctggtctgagaggatgaccagccacactggaactgagacacggtccagactcctacgggaggcagcagtggggaatattgcacaatgggcgcaagcctgatgcagccatgccgcgtgtatgaagaaggccttcgggttgtaaagtactttcagcggggaggaaggcgatacggttaataaccgtgtcgattgacgttacccgcagaagaagcaccggctaactccgtgccagcagccgcggtaatacggagggtgcaagcgttaatcggaattactgggcgtaaagcgcacgcaggcggtctgtcaagtcagatgtgaaatccccgggcttaacctgggaactgcatttgaaactggcaggcttgagtctcgtagaggggggtagaattccaggtgtagcggtgaaatgcgtagagatctggaggaataccggtggcgaaggcggccccctggacgaagactgacgctcaggtgcgaaagcgtggggagcaaacaggattagataccctggtagtccacgctgtaaacgatgtcgacttggaggttgtgcccttgaggcgtggcttccggagctaacgcgttaagtcgaccgcctggggagtacggccgcaaggttaaaactcaaatgaattgacgggggcccgcacaagcggtggagcatgtggtttaattcgatgcaacgcgaagaaccttacctggccttgacatccacagaattcggcagagatgccttagtgccttcgggaactgtgagacaggtgctgcatggctgtcgtcagctcgtgttgtgaaatgttgggttaagtcccgcaacgagcgcaacccttatcctttgttgccagcgattcggtcgggaactcaaaggagactgccggtgataaaccggaggaaggtggggatgacgtcaagtcatcatggcccttacggccagggctacacacgtgctacaatggcgcatacaaagagaagcgacctcgcgagagcaagcggacctcataaagtgcgtcgtagtccggatcggagtctgcaactcgactccgtgaagtcggaatcgctagtaatcgtagatcagaatgctacggtgaatacgttcccgggccttgtacacaccgcccgtcacaccatgggagtgggttgcaaaagaagtaggtagcttaaccttcgggagggcgcttaccactttgtgattcatgactggggtgaagtcgtaacaaggtaaccgtaggggaacctgcggttggatcacctcctt

SEQ ID NO: 24 - RNA, 1316 nt, unidentified
  source 1..1316: mol_type = rRNA; note = DP24 16S rRNA sequence
agcatttgattatggtgcttactgattgctatctaggggtttaacacatgctagtcaatgatcttttagattatggcgtacgggctaggaatacttagaatgataactctatgatcgcagtaatagcgtaaaaggtataataccgcatagaggttcgcttcgtatctcaataggtagttggtgaggtaaagctcaacaagccgatgatgagtaatattggatgaaagtcttaaatatagcagtggaaatgaaaaagtccaccgttatttattaacgcagcagtggagaatcgtcgtaatgtgcagtattcatttatggataagcatgaacgcgctacctagattcggataggagatagcatcttctaccgataaaagaacttagaataatgatctagttctcattagtgggtgacaatcgccgtgccagcatcagcggtaaaacggcttccgcaagcaatagtaatttaaattggtgtaaagggtacgtagccggccttattaggctagagttagatacgggtaagtacaatacttggagtagggctgatatcttatgatcccaaggggagtgctaaaggcgaaggcaacttactggtaataactgacggtgaggtacgaaggtcagggcatggaaagagattagatacctcattactcctgacagtaaacgatgtagattaaagattggaataattctgtcttaacgctaacgcattaaatctaccacctgtagagtatagtcgcaaggccgaaatacaaataattagacggctctagagcaaacggagtgaagcatgttatttaatacgataacccgcgtaaaatcttaccagttcttgaatcttagacaggtgttgcatggttgtcgtcagctcgtgctaatggtgtctggttaattccaaataacgagcgcaatccttacttctagttttctaggagtctccatttgacatacgtgtcaatggtttaaggaatatgacaaaccctcatggcccttatggactgggcaatagacgtgccacaagaatctagacaaaatgacgcgaaatggtaacaatgagctaatcatcaaagaagattaatgtacgaattatgggctggaactcgcccatatgaagtaggaattccgagtaatcgcgtatcagaacgacgcggtgaacatcatctctggagtgtactaactgctcgtcacgggacgaaagggagtgtattatgaagtggggctaattggttaactccggtgagtgtcacgaataatccttcccgattgttctgaagtcgaaacaaggtaaccgtaagggaacttgcggttga

SEQ ID NO: 25 - RNA, 1520 nt, unidentified
  source 1..1520: mol_type = rRNA; note = DP25 16S rRNA sequence
tacggagagtttgatcctggctcaggatgaacgctggcggcgtgcttaacacatgcaagtcgaacggtgaagccaagcttgcttggtggatcagtggcgaacgggtgagtaacacgtgagcaacctgccctggactctgggataagcgctggaaacggcgtctaatactggatatgagctccttccgcatggtgggggttggaaagatttttcggtctgggatgggctcgcggcctatcagcttgttggtgaggtaatggctcaccaaggcgtcgacgggtagccggcctgagagggtgaccggccacactgggactgagacacggcccagactcctacgggaggcagcagtggggaatattgcacaatgggcggaagcctgatgcagcaacgccgcgtgagggatgacggccttcgggttgtaaacctcttttagcagggaagaagcgaaagtgacggtacctgcagaaaaagcgccggctaactacgtgccagcagccgcggtaatacgtagggcgcaagcgttatccggaattattgggcgtaaagagctcgtaggcggtttgtcgcgtctgctgtgaaatcccgaggctcaacctcgggcctgcagtgggtacgggcagactagagtgcggtaggggagattggaattcctggtgtagcggtggaatgcgcagatatcaggaggaacaccgatggcgaaggcagatctctgggccgtaactgacgctgaggagcgaaagggtggggagcaaacaggcttagataccctggtagtccaccccgtaaacgttgggaactagttgtggggaccattccacggtttccgtgacgcagctaacgcattaagttccccgcctggggagtacggccgcaaggctaaaactcaaaggaattgacggggacccgcacaagcggcggagcatgcggattaattcgatgcaacgcgaagaaccttaccaaggcttgacatatacgagaacgggccagaaatggtcaactctttggacactcgtaaacaggtggtgcatggttgtcgtcagctcgtgtcgtgagatgttgggttaagtcccgcaacgagcgcaaccctcgttctatgttgccagcacgtaatggtgggaactcatgggatactgccggggtcaactcggaggaaggtggggatgacgtcaaatcatcatgccccttatgtcttgggcttcacgcatgctacaatggccggtacaaagggctgcaataccgtaaggtggagcgaatcccaaaaagccggtcccagttcggattgaggtctgcaactcgacctcatgaagtcggagtcgctagtaatcgcagatcagcaacgctgcggtgaatacgttcccgggtcttgtacacaccgcccgtcaagtcatgaaagtcggtaacacctgaagccggtggcccaacccttgtggagggagccgtcgaaggtgggatcggtaattaggactaagtcgtaacaaggtagccgtaccggaaggtgcggctggatcacctccttt

SEQ ID NO: 26 - RNA, 1482 nt, unidentified
  source 1..1482: mol_type = rRNA; note = DP26 16S rRNA sequence
cttgagagtttgatcctggctcagagcgaacgctggcggcaggcttaacacatgcaagtcgagcgggcatcttcggatgtcagcggcagacgggtgagtaacacgtgggaacgtacccttcggttcggaataacgctgggaaactagcgctaataccggatacgcccttttggggaaaggtttactgccgaaggatcggcccgcgtctgattagctagttggtggggtaacggcctaccaaggcgacgatcagtagctggtctgagaggatgatcagccacactgggactgagacacggcccagactcctacgggaggcagcagtggggaatattggacaatgggcgcaagcctgatccagccatgccgcgtgagtgatgaaggccttagggttgtaaagctcttttgtccgggacgataatgacggtaccggaagaataagccccggctaacttcgtgccagcagccgcggtaatacgaagggggctagcgttgctcggaatcactgggcgtaaagggcgcgtaggcggccattcaagtcgggggtgaaagcctgtggctcaaccacagaattgccttcgatactgtttggcttgagtatggtagaggttggtggaactgcgagtgtagaggtgaaattcgtagatattcgcaagaacaccggtggcgaaggcggccaactggaccattactgacgctgaggcgcgaaagcgtggggagcaaacaggattagataccctggtagtccacgccgtaaacgatgaatgccagctgttggggtgcttgcacctcagtagcgcagctaacgctttaagcattccgcctggggagtacggtcgcaagattaaaactcaaaggaattgacgggggcccgcacaagcggtggagcatgtggtttaattcgaagcaacgcgcagaaccttaccatcccttgacatggcatgttacccggagagattcggggtccacttcggtggcgtgcacacaggtgctgcatggctgtcgtcagctcgtgtcgtgagatgttgggttaagtcccgcaacgagcgcaacccacgtccttagttgccatcattcagttgggcactctagggagactgccggtgataagccgcgaggaaggtgtggatgacgtcaagtcctcatggcccttacgggatgggctacacacgtgctacaatggcggtgacagtgggacgcgaaggagcgatctggagcaaatccccaaaaaccgtctcagttcagattgcactctgcaactcgagtgcatgaaggcggaatcgctagtaatcgtggatcagcatgccacggtgaatacgttcccgggccttgtacacaccgcccgtcacaccatgggagttggtcttacccgacggcgctgcgccaaccgcaaggaggcaggcgaccacggtagggtcagcgactggggtgaagtcgtaacaaggtagccgtaggggaacctgcggctggatcacctccttt

SEQ ID NO: 27 - RNA, 935 nt, unidentified
  source 1..935: mol_type = rRNA; note = DP27 16S rRNA sequence
cttgagagtttgatcctggctcagaacgaacgctggcggcatgcctaacacatgcaagtcgaacgatgctttcgggcatagtggcgcacgggtgcgtaacgcgtgggaatctgccctcaggttcggaataacagctggaaacggctgctaataccggatgatatcgcaagatcaaagatttatcgcctgaggatgagcccgcgttggattaggtagttggtggggtaaaggcctaccaagccgacgatccatagctggtctgagaggatgatcagccacactgggactgagacacggcccagactcctacgggaggcagcagtggggaatattggacaatgggcgcaagcctgatccagcaatgccgcgtgagtgatgaaggccctagggttgtaaagctcttttacccgggaagataatgactgtaccgggagaataagccccggctaactccgtgccagcagccgcggtaatacggagggggctagcgttgttcggaattactgggcgtaaagcgcacgtaggcggctttgtaagtcagaggtgaaagcctggagctcaactccagaactgcctttgagactgcatcgcttgaatccaggagaggtcagtggaattccgagtgtagaggtgaaattcgtagatattcggaagaacaccagtggcgaaggcggctgactggactggtattgacgctgaggtgcgaaagcgtggggagcaaacaggattagataccctggtagtccacgccgtaaacgatgataactagctgtccgggcacttggtgcttgggtggcgcagctaacgcattaagttatccgcctggggagtacggccgcaaggttaaaactcaaaggaattgacgggggcctgcacaagcggtggagcatgtggtttaattcgaagcaacgcgcagaaccttaccagcgtttgac

SEQ ID NO: 28 - RNA, 931 nt, unidentified
  source 1..931: mol_type = rRNA; note = DP28 16S rRNA sequence
atagtcgggggcatcagtattcaattgtcagaggtgaaattcttggatttattgaagactaactactgcgaaagcatttgccaaggatgttttcattaatcagtgaacgaaagttaggggatcgaagacgatcagataccgtcgtagtcttaaccataaactatgccgactagggatcgggcgatgttatcattttgactcgctcggcaccttacgagaaatcaaagtctttgggttctggggggagtatggtcgcaaggctgaaacttaaagaaattgacggaagggcaccaccaggcgtggagcctgcggcttaatttgactcaacacggggaaactcaccaggtccagacacaataaggattgacagattgagagctctttcttgattttgtgggtggtggtgcatggccgttcttagttggtggagtgatttgtctgcttaattgcgataacgaacgagaccttaacctgctaaatagcccggcccgctttggcgggtcgccggcttcttagagggactatcggctcaagccgatggaagtttgaggcaataacaggtctgtgatgcccttagatgttctgggccgcacgcgcgctacactgacagagccaacgagttcatttccttgcccggaagggttgggtaatcttgttaaactctgtcgtgctggggatagagcattgcaattattgctcttcaacgaggaatgcctagtaagcgtacgtcatcagcgtgcgttgattacgtccctgccctttgtacacaccgcccgtcgctactaccgattgaatggctgagtgaggccttcggactggcccagggaggtcggcaacgaccacccagggccggaaagttggtcaaactccgtcatttagaggaagtaaaagtcgtaacaaggtttccgtaggtgaacctgcggaaggatca

SEQ ID NO: 29 - RNA, 1002 nt, unidentified
  source 1..1002: mol_type = rRNA; note = DP29 16S rRNA sequence
tacggagagtttgatcctggctcaggacgaacgctggcggcgtgcttaacacatgcaagtcgaacgatgaagcccagcttgctgggttgattagtggcgaacgggtgagtaacacgtgagcaacgtgcccataactctgggataacctccggaaacggtggctaatactggatatctaacacgatcgcatggtctgtgtttggaaagattttttggttatggatcggctcacggcctatcagcttgttggtgaggtaatggctcaccaaggcgacgacgggtagccggcctgagagggtgaccggccacactgggactgagacacggcccagactcctacgggaggcagcagtggggaatattgcacaatgggcgaaagcctgatgcagcaacgccgcgtgagggatgacggcattcgggttgtaaacctcttttagtagggaagaagcgaaagtgacggtacctgcagaaaaagcaccggctaactacgtgccagcagccgctgtaatacgtagggtgcaagcgttgtccggaattattgggcgtaaagagctcgtaggcggtttgtcgcgtctgctgtgaaatcccgaggctcaacctcgggtctgcagtgggtacgggcagactagagtgtggtaggggagattggaattcctggtgtagcggtggaatgcgcagatatcaggaggaacaccgatggcgaaggcagatctctgggccattactgacgctgaggagcgaaagcatggggagcgaacaggattagataccctggtagtccatgccgtaaacgttgggcgctagatgtggggaccattccacggtttccgtgtcgtagctaacgcattaagcgccccgcctggggagtacggccgcaaggctaaaactcaaaggaattgacgggggcccgcacaagcggcggagcatgcggattaattcgatgcaacgcgaagaaccttaccaaggcttgacatataccggaaacgttcagaaatgttcgcc

SEQ ID NO: 30 - RNA, 1520 nt, unidentified
  misc_feature 1..1520: note = DP30 16S rRNA sequence
  source 1..1520: mol_type = rRNA; note = DP30 16S rRNA sequence
tacggagagtttgatcctggctcaggatgaacgctggcggcgtgcttaacacatgcaagtcgaacggtgaagccaagcttgcttggtggatcagtggcgaacgggtgagtaacacgtgagcaacctgccctggactctgggataagcgctggaaacggcgtctaatactggatatgagacgtgatcgcatggtcgtgtttggaaagatttttcggtctgggatgggctcgcggcctatcagcttgttggtgaggtaatggctcaccaaggcgtcgacgggtagccggcctgagagggtgaccggccacactgggactgagacacggcccagactcctacgggaggcagcagtggggaatattgcacaatgggcgaaagcctgatgcagcaacgccgcgtgagggatgacggccttcgggttgtaaacctcttttagcagggaagaagcgaaagtgacggtacctgcagaaaaagcgccggctaactacgtgccagcagccgcggtaatacgtagggcgcaagcgttatccggaattattgggcgtaaagagctcgtaggcggtttgtcgcgtctgctgtgaaatcccgaggctcaacctcgggcctgcagtgggtacgggcagactagagtgcggtaggggagattggaattcctggtgtagcggtggaatgcgcagatatcaggaggaacaccgatggcgaaggcagatctctgggccgtaactgacgctgaggagcgaaagggtggggagcaaacaggcttagataccctggtagtccaccccgtaaacgttgggaactagttgtggggaccattccacggtttccgtgacgcagctaacgcattaagttccccgcctggggagtacggccgcaaggctaaaactcaaaggaattgacggggacccgcacaagcggcggagcatgcggattaattcgatgcaacgcgaagaaccttaccaaggcttgacatatacgagaacgggccagaaatggtcaactctttggacactcgtaaacaggtggtgcatggttgtcgtcagctcgtgtcgtgagatgttgggttaagtcccgcaacgagcgcaaccctcgttctatgttgccagcacgtaatggtgggaactcatgggatactgccggggtcaactcggaggaaggtggggatgacgtcaaatcatcatgccccttatgtcttgggcttcacgcatgctacaatggccggtacaaagggctgcaataccgtgaggtggagcgaatcccaaaaagccggtcccagttcggattgaggtctgcaactcgacctcatgaagtcggagtcgctagtaatcgcagatcagcaacgctgcggtgaatacgttcccgggtcttgtacacaccgcccgtcaagtcatgaaagtcggtaacacctgaagccggtggcccaacccttgtggagggagccgtcgaaggtgggatcggtaattaggactaagtcgtaacaaggtagccgtaccggaaggtgcggctggatcacctccttt

SEQ ID NO: 31 - RNA, 941 nt, unidentified
  source 1..941: mol_type = rRNA; note = DP31 16S rRNA sequence
cagccgggggcattagtatttgcacgctagaggtgaaattcttggattgtgcaaagacttcctactgcgaaagcatttgccaagaatgttttcattaatcaagaacgaaggttagggtatcgaaaacgattagataccgttgtagtcttaacagtaaactatgccgactccgaatcggtcgatgctcatttcactggctcgatcggcgcggtacgagaaatcaaagtttttgggttctggggggagtatggtcgcaaggctgaaacttaaagaaattgacggaagggcaccaccaggagtggagcctgcggcttaatttgactcaacacgggaaaactcaccgggtccggacatagtaaggattgacagattgatggcgctttcatgattctatgggtggtggtgcatggccgttcttagttggtggagtgatttgtctggttaattccgataacgaacgagaccttgacctgctaaatagacgggttgacattttgttggccccttatgtcttcttagagggacaatcgaccgtctaggtgatggaggcaaaaggcaataacaggtctgtgatgcccttagatgttccgggctgcacgcgcgctacactgacagagacaacgagtggggccccttgtccgaaatgactgggtaaacttgtgaaactttgtcgtgctggggatggagctttgtaatttttgctcttcaacgaggaattcctagtaagcgcaagtcatcagcttgcgttgactacgtccctgccctttgtacacaccgcccgtcgctactaccgattgaatggcttagtgaggacttgggagagtacatcggggagccagcaatggcaccctgacggctcaaactcttacaaacttggtcatttagaggaagtaaaagtcgtaacaaggtatctgtaggtgaacctgcagatggatcatttc

SEQ ID NO: 32 - RNA, 1072 nt, unidentified
  source 1..1072: mol_type = rRNA; note = DP32 16S rRNA sequence
actgagcattgacgttactcgcagaagaagcaccggctaactccgtgccagcagccgcggtaatacggagggtgcaagcgttaatcggaattactgggcgtaaagcgcacgcaggcggtttgttaagtcagatgtgaaatccccgagcttaacttgggaactgcatttgaaactggcaagctagagtcttgtagaggggggtagaattccaggtgtagcggtgaaatgcgtagagatctggaggaataccggtggcgaaggcggccccctggacaaagactgacgctcaggtgcgaaagcgtggggagcaaacaggattagataccctggtagtccacgctgtaaacgatgtcgacttggaggttgtgcccttgaggcgtggcttccggagctaacgcgttaagtcgaccgcctggggagtacggccgcaaggttaaaactcaaatgaattgacgggggcccgcacaagcggtggagcatgtggtttaattcgatgcaacgcgaagaaccttacctactcttgacatccagagaattcgctagagatagcttagtgccttcgggaactctgagacaggtgctgcatggctgtcgtcagctcgtgttgtgaaatgttgggttaagtcccgcaacgagcgcaacccttatcctttgttgccagcgagtaatgtcgggaactcaaaggagactgccggtgataaaccggaggaaggtggggatgacgtcaagtcatcatggcccttacgagtagggctacacacgtgctacaatggcatatacaaagagaagcgaactcgcgagagcaagcggacctcataaagtatgtcgtagtccggattggagtctgcaactcgactccatgaagtcggaatcgctagtaatcgtagatcagaatgctacggtgaatacgttcccgggccttgtacacaccgcccgtcacaccatgggagtgggttgcaaaagaagtaggtagcttaaccttcgggagggcgcttaccactttgtgattcatgactggggtgaagtcgtaacaaggtaaccgtaggggaacctgcggttggatcacctcctt

SEQ ID NO: 33 - RNA, 853 nt, unidentified
  source 1..853: mol_type = rRNA; note = DP33 16S rRNA sequence
ggaggaaggcgtagagatctggaggaataccggtggcgaaggcggccccctggacaaagactgacgctcaggtgcgaaagcgtggggagcaaacaggattagataccctggtagtccacgccgtaaacgatgtcgacttggaggttgtgcccttgaggcgtggcttccggagctaacgcgttaagtcgaccgcctggggagtacggccgcaaggttaaaactcaaatgaattgacgggggcccgcacaagcggtggagcatgtggtttaattcgatgcaacgcgaagaaccttacctggccttgacatccacggaattcggcagagatgccttagtgccttcgggaaccgtgagacaggtgctgcatggctgtcgtcagctcgtgttgtgaaatgttgggttaagtcccgcaacgagcgcaacccttatcctttgttgccagcacgtaatggtgggaactcaaaggagactgccggtgataaaccggaggaaggtggggatgacgtcaagtcatcatggcccttacggccagggctacacacgtgctacaatggcgcatacaaagagaagcgacctcgcgagagcaagcggacctcataaagtgcgtcgtagtccggatcggagtctgcaactcgactccgtgaagtcggaatcgctagtaatcgtagatcagaatgctacggtgaatacgttcccgggccttgtacacaccgcccgtcacaccatgggagtgggttgcaaaagaagtaggtagcttaaccttcgggagggcgcttaccactttgtgattcatgactggggtgaagtcgtaacaaggtaaccgtaggggaacctgcggttggatcacctcctt

SEQ ID NO: 34 - RNA, 1519 nt, unidentified
  source 1..1519: mol_type = rRNA; note = DP34 16S rRNA sequence
tacggagagtttgatcctggctcaggacgaacgctggcggcgtgcttaacacatgcaagtcgaacgatgaagcccagcttgctgggtggattagtggcgaacgggtgagtaacacgtgagtaacctgcccttgactctgggataagcgttggaaacgacgtctaataccggatacgagcttccaccgcatggtgagttgctggaaagaattttggtcaaggatggactcgcggcctatcagcttgttggtgaggtaatggctcaccaaggcgacgacgggtagccggcctgagagggtgaccggccacactgggactgagacacggcccagactcctacgggaggcagcagtggggaatattgcacaatgggcgaaagcctgatgcagcaacgccgcgtgagggacgacggccttcgggttgtaaacctcttttagcagggaagaagcgaaagtgacggtacctgcagaaaaagcaccggctaactacgtgccagcagccgcggtaatacgtagggtgcaagcgttgtccggaattattgggcgtaaagagctcgtaggcggtttgtcgcgtctgctgtgaaatcccgaggctcaacctcgggtctgcagtgggtacgggcagactagagtgcggtaggggagattggaattcctggtgtagcggtggaatgcgcagatatcaggaggaacaccgatggcgaaggcagatctctgggccgctactgacgctgaggagcgaaagggtggggagcaaacaggcttagataccctggtagtccaccccgtaaacgttgggcgctagatgtggggaccattccacggtttccgtgtcgtagctaacgcattaagcgccccgcctggggagtacggccgcaaggctaaaactcaaaggaattgacgggggcccgcacaagcggcggagcatgcggattaattcgatgcaacgcgaagaaccttaccaaggcttgacatatacgagaacgggccagaaatggtcaactctttggacactcgtaaacaggtggtgcatggttgtcgtcagctcgtgtcgtgagatgttgggttaagtcccgcaacgagcgcaaccctcgttctatgttgccagcacgtaatggtgggaactcatgggatactgccggggtcaactcggaggaaggtggggacgacgtcaaatcatcatgccccttatgtcttgggcttcacgcatgctacaatggccagtacaaagggctgcaataccgtaaggtggagcgaatcccaaaaagctggtcccagttcggattgaggtctgcaactcgacctcatgaagtcggagtcgctagtaatcgcagatcagcaacgctgcggtgaatacgttcccgggccttgtacacaccgcccgtcaagtcatgaaagtcggtaacacccgaagccagtggcctaaccgcaaggatggagctgtctaaggtgggatcggtaattaggactaagtcgtaacaaggtagccgtaccggaaggtgcggctggatcacctccttt

SEQ ID NO: 35 - RNA, 1536 nt, unidentified
  source 1..1536: mol_type = rRNA; note = DP35 16S rRNA sequence
ttgaagagtttgatcatggctcagattgaacgctggcggcaggcctaacacatgcaagtcggacggtagcacagagagcttgctcttgggtgacgagtggcggacgggtgagtaatgtctggggatctgcccgatagagggggataaccactggaaacggtggctaataccgcataacgtcgcaagaccaaagagggggaccttcgggcctctcactatcggatgaacccagatgggattagctagtaggcggggtaatggcccacctaggcgacgatccctagctggtctgagaggatgaccagccacactggaactgagacacggtccagactcctacgggaggcagcagtggggaatattgcacaatgggcgcaagcctgatgcagccatgccgcgtgtatgaagaaggccttcgggttgtaaagtactttcagcggggaggaaggcgatgaggttaataaccgcgtcgattgacgttacccgcagaagaagcaccggctaactccgtgccagcagccgcggtaatacggagggtgcaagcgttaatcggaattactgggcgtaaagcgcacgcaggcggtctgttaagtcagatgtgaaatccccgggcttaacctgggaactgcatttgaaactggcaggcttgagtcttgtagaggggggtagaattccaggtgtagcggtgaaatgcgtagagatctggaggaataccggtggcgaaggcggccccctggacaaagactgacgctcaggtgcgaaagcgtggggagcaaacaggattagataccctggtagtccacgccgtaaacgatgtcgacttggaggttgttcccttgaggagtggcttccggagctaacgcgttaagtcgaccgcctggggagtacggccgcaaggttaaaactcaaatgaattgacgggggcccgcacaagcggtggagcatgtggtttaattcgatgcaacgcgaagaaccttacctactcttgacatccagcgaacttagcagagatgctttggtgccttcgggaacgctgagacaggtgctgcatggctgtcgtcagctcgtgttgtgaaatgttgggttaagtcccgcaacgagcgcaacccttatcctttgttgccagcgattcggtcgggaactcaaaggagactgccggtgataaaccggaggaaggtggggatgacgtcaagtcatcatggcccttacgagtagggctacacacgtgctacaatggcgcatacaaagagaagcgacctcgcgagagcaagcggacctcacaaagtgcgtcgtagtccggatcggagtctgcaactcgactccgtgaagtcggaatcgctagtaatcgtggatcagaatgccacggtgaatacgttcccgggccttgtacacaccgcccgtcacaccatgggagtgggttgcaaaagaagtaggtagcttaaccttcgggagggcgcttaccactttgtgattcattactggggtgaagtcgtaacaaggtaaccgtaggggaacctgcggttggatcacctcctt

SEQ ID NO: 36 - RNA, 993 nt, unidentified
  source 1..993: mol_type = rRNA; note = DP36 16S rRNA sequence
ttgaagagtttgatcatggctcagattgaacgctggcggcaggcctaacacatgcaagtcggacggtagcacagagagcttgctcttgggtgacgagtggcggacgggtgagtaatgtctggggatctgcccgatagagggggataaccactggaaacggtggctaataccgcataacgtcgcaagaccaaagagggggaccttcgggcctctcactatcggatgaacccagatgggattagctagtaggcggggtaatggcccacctaggcgacgatccctagctggtctgagaggatgaccagccacactggaactgagacacggtccagactcctacgggaggcagcagtggggaatattgcacaatgggcgcaagcctgatgcagccatgccgcgtgtatgaagaaggccttcgggttgtaaagtactttcagcggggaggaaggcgatgcggttaataaccgcgtcgattgacgttacccgcagaagaagcaccggctaactccgtgccagcagccgcggtaatacggagggtgcaagcgttaatcggaattactgggcgtaaagcgcacgcaggcggtctgttaagtcagatgtgaaatccccgggcttaacctgggaactgcatttgaaactggcaggcttgagtcttgtagaggggggtagaattccaggtgtagcggtgaaatgcgtagagatctggaggaataccggtggcgaaggcggccccctggacaaagactgacgctcaggtgcgaaagcgtggggagcaaacaggattagataccctggtagtccacgccgtaaacgatgtcgacttggaggttgttcccttgaggagtggcttccggagctaacgcgttaagtcgaccgcctggggagtacggccgcaaggttaaaactcaaatgaattgacgggggcccgcacaagcggtggagcatgtggtttaattcgatgcaacgcgaagaaccttacctactcttgacatc

SEQ ID NO: 37 - RNA, 1532 nt, unidentified
  source 1..1532: mol_type = rRNA; note = DP37 16S rRNA sequence
tgaagagtttgatcatggctcagattgaacgctggcggcaggcctaacacatgcaagtcgagcggtagagagaagcttgcttctcttgagagcggcggacgggtgagtaatgcctaggaatctgcctggtagtgggggataacgttcggaaacgaacgctaataccgcatacgtcctacgggagaaagcaggggaccttcgggccttgcgctatcagatgagcctaggtcggattagctagttggtggggtaatggctcaccaaggcgacgatccgtaactggtctgagaggatgatcagtcacactggaactgagacacggtccagactcctacgggaggcagcagtggggaatattggacaatgggcgaaagcctgatccagccatgccgcgtgtgtgaagaaggtcttcggattgtaaagcactttaagttgggaggaagggccattacctaatacgtgatggttttgacgttaccgacagaataagcaccggctaactctgtgccagcagccgcggtaatacagagggtgcaagcgttaatcggaattactgggcgtaaagcgcgcgtaggtggtttgttaagttggatgtgaaatccccgggctcaacctgggaactgcattcaaaactgactgactagagtatggtagagggtggtggaatttcctgtgtagcggtgaaatgcgtagatataggaaggaacaccagtggcgaaggcgaccacctggactgatactgacactgaggtgcgaaagcgtggggagcaaacaggattagataccctggtagtccacgccgtaaacgatgtcaactagccgttgggagccttgagctcttagtggcgcagctaacgcattaagttgaccgcctggggagtacggccgcaaggttaaaactcaaatgaattgacgggggcccgcacaagcggtggagcatgtggtttaattcgaagcaacgcgaagaaccttaccaggccttgacatccaatgaactttctagagatagattggtgccttcgggaacattgagacaggtgctgcatggctgtcgtcagctcgtgtcgtgagatgttgggttaagtcccgtaacgagcgcaacccttgtccttagttaccagcacgtaatggtgggcactctaaggagactgccggtgacaaaccggaggaaggtggggatgacgtcaagtcatcatggcccttacggcctgggctacacacgtgctacaatggtcggtacagagggttgccaagccgcgaggtggagctaatcccataaaaccgatcgtagtccggatcgcagtctgcaactcgactgcgtgaagtcggaatcgctagtaatcgcgaatcagaatgtcgcggtgaatacgttcccgggccttgtacacaccgcccgtcacaccatgggagtgggttgcaccagaagtagctagtctaaccttcggggggacggttaccacggtgtgattcatgactggggtgaagtcgtaacaaggtagccgtaggggaacctgcggctggatcacctcctt

SEQ ID NO: 38 - RNA, 1517 nt, unidentified
  source 1..1517: mol_type = rRNA; note = DP38 16S rRNA sequence
tacggagagtttgatcctggctcaggacgaacgctggcggcgtgcttaacacatgcaagtcgagcggtaaggcctttcggggtacacgagcggcgaacgggtgagtaacacgtgggtgatctgccctgcactctgggataagcttgggaaactgggtctaataccggatatgaccacagcatgcatgtgttgtggtggaaagatttatcggtgcaggatgggcccgcggcctatcagcttgttggtggggtaatggcctaccaaggcgacgacgggtagccgacctgagagggtgaccggccacactgggactgagacacggcccagactcctacgggaggcagcagtggggaatattgcacaatgggcggaagcctgatgcagcgacgccgcgtgagggatgaaggccttcgggttgtaaacctctttcagcagggacgaagcgtgagtgacggtacctgcagaagaagcaccggctaactacgtgccagcagccgcggtaatacgtagggtgcgagcgttgtccggaattactgggcgtaaagagttcgtaggcggtttgtcgcgtcgtttgtgaaaacccggggctcaacttcgggcttgcaggcgatacgggcagacttgagtgtttcaggggagactggaattcctggtgtagcggtgaaatgcgcagatatcaggaggaacaccggtggcgaaggcgggtctctgggaaacaactgacgctgaggaacgaaagcgtgggtagcaaacaggattagataccctggtagtccacgccgtaaacggtgggcgctaggtgtgggttccttccacgggatctgtgccgtagctaacgcattaagcgccccgcctggggagtacggccgcaaggctaaaactcaaaggaattgacgggggcccgcacaagcggcggagcatgtggattaattcgatgcaacgcgaagaaccttacctgggtttgacatacaccggaaaaccgtagagatacggtcccccttgtggtcggtgtacaggtggtgcatggctgtcgtcagctcgtgtcgtgagatgttgggttaagtcccgcaacgagcgcaacccttgtcttatgttgccagcacgtaatggtggggactcgtaagagactgccggggtcaactcggaggaaggtggggacgacgtcaagtcatcatgccccttatgtccagggcttcacacatgctacaatggccagtacagagggctgcgagaccgtgaggtggagcgaatcccttaaagctggtctcagttcggatcggggtctgcaactcgaccccgtgaagtcggagtcgctagtaatcgcagatcagcaacgctgcggtgaatacgttcccgggccttgtacacaccgcccgtcacgtcatgaaagtcggtaacacccgaagccggtggcctaaccccttacggggagggagccgtcgaaggtgggatcggcgattgggacgaagtcgtaacaaggtagccgtaccggaaggtgcggctggatcacctccttt

SEQ ID NO: 39 - RNA, 1482 nt, unidentified
  source 1..1482: mol_type = rRNA; note = DP39 16S rRNA sequence
cttgagagtttgatcctggctcagaacgaacgctggcggcaggcttaacacatgcaagtcgaacgccccgcaaggggagtggcagacgggtgagtaacgcgtgggaatctaccgtgccctgcggaatagctccgggaaactggaattaataccgcatacgccctacgggggaaagatttatcggggtatgatgagcccgcgttggattagctagttggtggggtaaaggcctaccaaggcgacgatccatagctggtctgagaggatgatcagccacattgggactgagacacggcccaaactcctacgggaggcagcagtggggaatattggacaatgggcgcaagcctgatccagccatgccgcgtgagtgatgaaggccttagggttgtaaagctctttcaccggagaagataatgacggtatccggagaagaagccccggctaacttcgtgccagcagccgcggtaatacgaagggggctagcgttgttcggaattactgggcgtaaagcgcacgtaggcggatatttaagtcaggggtgaaatcccagagctcaactctggaactgcctttgatactgggtatcttgagtatggaagaggtaagtggaattccgagtgtagaggtgaaattcgtagatattcggaggaacaccagtggcgaaggcggcttactggtccattactgacgctgaggtgcgaaagcgtggggagcaaacaggattagataccctggtagtccacgccgtaaacgatgaatgttagccgtcgggcagtatactgttcggtggcgcagctaacgcattaaacattccgcctggggagtacggtcgcaagattaaaactcaaaggaattgacgggggcccgcacaagcggtggagcatgtggtttaattcgaagcaacgcgcagaaccttaccagctcttgacattcggggtttgggcagtggagacattgtccttcagttaggctggccccagaacaggtgctgcatggctgtcgtcagctcgtgtcgtgagatgttgggttaagtcccgcaacgagcgcaaccctcgcccttagttgccagcatttagttgggcactctaaggggactgccggtgataagccgagaggaaggtggggatgacgtcaagtcctcatggcccttacgggctgggctacacacgtgctacaatggtggtgacagtgggcagcgagacagcgatgtcgagctaatctccaaaagccatctcagttcggattgcactctgcaactcgagtgcatgaagttggaatcgctagtaatcgcagatcagcatgctgcggtgaatacgttcccgggccttgtacacaccgcccgtcacaccatgggagttggttttacccgaaggtagtgcgctaaccgcaaggaggcagctaaccacggtagggtcagcgactggggtgaagtcgtaacaaggtagccgtaggggaacctgcggctggatcacctccttt

SEQ ID NO: 40 - RNA, 903 nt, unidentified
  source 1..903: mol_type = rRNA; note = DP40 16S rRNA sequence
ttgacgttacccgcagaagaagcaccggctaactccgtgccagcagccgcggtaatacggagggtgcaagcgttaatcggaattactgggcgtaaagcgcacgcaggcggtctgttaagtcagatgtgaaatccccgggcttaacctgggaactgcatttgaaactggcaggcttgagtcttgtagaggggggtagaattccaggtgtagcggtgaaatgcgtagagatctggaggaataccggtggcgaaggcggccccctggacaaagactgacgctcaggtgcgaaagcgtggggagcaaacaggattagataccctggtagtccacgccgtaaacgatgtcgacttggaggttgttcccttgaggagtggcttccggagctaacgcgttaagtcgaccgcctggggagtacggccgcaaggttaaaactcaaatgaattgacgggggcccgcacaagcggtggagcatgtggtttaattcgatgcaacgcgaagaaccttacctactcttgacatccagagaactttccagagatggattggtgccttcgggaactctgagacaggtgctgcatggctgtcgtcagctcgtgttgtgaaatgttgggttaagtcccgcaacgagcgcaacccttatcctttgttgccagcgcgtgatggcgggaactcaaaggagactgccggtgataaaccggaggaaggtggggatgacgtcaagtcatcatggcccttacgagtagggctacacacgtgctacaatggcgcatacaaagagaagcgacctcgcgagagcaagcggacctcacaaagtgcgtcgtagtccggatcggagtctgcaactcgactccgtgaagtcggaatcgctagtaatcgtggatcagaatgccacggtgaatacgt

SEQ ID NO: 41 - RNA, 1457 nt, unidentified
  source 1..1457: mol_type = rRNA; note = DP41 16S rRNA sequence
gtggagagtttgatcctggctcaggatgaacgctggcggcgtgcttaacacatgcaagtcgaacggaaaggcccaagcttgcttgggtactcgagtggcgaacgggtgagtaacacgtgggtgatctgccctgcacttcgggataagcctgggaaactgggtctaataccggataggacgatggtttggatgccattgtggaaagttttttcggtgtgggatgagctcgcggcctatcagcttgttggtggggtaatggcctaccaaggcgtcgacgggtagccggcctgagagggtgtacggccacattgggactgagatacggcccagactcctacgggaggcagcagtggggaatattgcacaatgggcgcaagcctgatgcagcgacgccgcgtgggggatgacggccttcgggttgtaaactcctttcgctagggacgaagcgttttgtgacggtacctggagaagaagcaccggctaactacgtgccagcagccgcggtaatacgtagggtgcgagcgttgtccggaattactgggcgtaaagagctcgtaggtggtttgtcgcgtcgtttgtgtaagcccgcagcttaactgcgggactgcaggcgatacgggcataacttgagtgctgtaggggagactggaattcctggtgtagcggtggaatgcgcagatatcaggaggaacaccgatggcgaaggcaggtctctgggcagtaactgacgctgaggagcgaaagcatgggtagcgaacaggattagataccctggtagtccatgccgtaaacggtgggcgctaggtgtgagtcccttccacggggttcgtgccgtagctaacgcattaagcgccccgcctggggagtacggccgcaaggctaaaactcaaaggaattgacgggggcccgcacaagcggcggagcatgtggattaattcgatgcaacgcgaagaaccttacctgggcttgacatacaccagatcgccgtagagatacggtttccctttgtggttggtgtacaggtggtgcatggttgtcgtcagctcgtgtcgtgagatgttgggttaagtcccgcaacgagcgcaacccttgtcttatgttgccagcacgtgatggtggggactcgtgagagactgccggggttaactcggaggaaggtggggatgacgtcaaatcatcatgccccttatgtccagggcttcacacatgctacaatggtcggtacaacgcgcatgcgagcctgtgagggtgagcgaatcgctgtgaaagccggtcgtagttcggattggggtctgcaactcgaccccatgaagtcggagtcgctagtaatcgcagatcagcaacgctgcggtgaatacgttcccgggccttgtacacaccgcccgtcacaccatgggagtgggttgcaaaagaagtaggtagcttaaccttcgggagggcgcttaccactttgtgat

SEQ ID NO: 42 - RNA, 1532 nt, unidentified
  source 1..1532: mol_type = rRNA; note = DP42 16S rRNA sequence
tgaagagtttgatcatggctcagattgaacgctggcggcaggcctaacacatgcaagtcgagcggtagagaggtgcttgcacctcttgagagcggcggacgggtgagtaatacctaggaatctgcctgatagtgggggataacgttcggaaacggacgctaataccgcatacgtcctacgggagaaagcaggggaccttcgggccttgcgctatcagatgagcctaggtcggattagctagttggtgaggtaatggctcaccaaggctacgatccgtaactggtctgagaggatgatcagtcacactggaactgagacacggtccagactcctacgggaggcagcagtggggaatattggacaatgggcgaaagcctgatccagccatgccgcgtgtgtgaagaaggtcttcggattgtaaagcactttaagttgggaggaagggcattaacctaatacgttagtgtcttgacgttaccgacagaataagcaccggctaactctgtgccagcagccgcggtaatacagagggtgcaagcgttaatcggaattactgggcgtaaagcgcgcgtaggtggtttgttaagttgaatgtgaaatccccgggctcaacctgggaactgcatccaaaactggcaagctagagtatggtagagggtagtggaatttcctgtgtagcggtgaaatgcgtagatataggaaggaacaccagtggcgaaggcgactacctggactgatactgacactgaggtgcgaaagcgtggggagcaaacaggattagataccctggtagtccacgccgtaaacgatgtcaactagccgttgggaaccttgagttcttagtggcgcagctaacgcattaagttgaccgcctggggagtacggccgcaaggttaaaactcaaatgaattgacgggggcccgcacaagcggtggagcatgtggtttaattcgaagcaacgcgaagaaccttaccaggccttgacatccaatgaactttccagagatggattggtgccttcgggaacattgagacaggtgctgcatggctgtcgtcagctcgtgtcgtgagatgttgggttaagtcccgtaacgagcgcaacccttgtccttagttaccagcacgtaatggtgggcactctaaggagactgccggtgacaaaccggaggaaggtggggatgacgtcaagtcatcatggcccttacggcctgggctacacacgtgctacaatggtcggtacaaagggttgccaagccgcgaggtggagctaatcccataaaaccgatcgtagtccggatcgcagtctgcaactcgactgcgtgaagtcggaatcgctagtaatcgtgaatcagaatgtcacggtgaatacgttcccgggccttgtacacaccgcccgtcacaccatgggagtgggttgcaccagaagtagctagtctaaccctcgggaggacggttaccacggtgtgattcatgactggggtgaagtcgtaacaaggtagccgtaggggaacctgcggctggatcacctcctt

SEQ ID NO: 43 - RNA, 1525 nt, unidentified
  source 1..1525: mol_type = rRNA; note = DP43 16S rRNA sequence
ctgagtttgatcctggctcagattgaacgctggcggcatgccttacacatgcaagtcgaacggcagcacggagcttgctctggtggcgagtggcgaacgggtgagtaatatatcggaacgtaccctggagtgggggataacgtagcgaaagttacgctaataccgcatacgatctaaggatgaaagtgggggatcgcaagacctcatgctcgtggagcggccgatatctgattagctagttggtagggtaaaagcctaccaaggcatcgatcagtagctggtctgagaggacgaccagccacactggaactgagacacggtccagactcctacgggaggcagcagtggggaattttggacaatgggcgaaagcctgatccagcaatgccgcgtgagtgaagaaggccttcgggttgtaaagctcttttgtcagggaagaaacggtgagagctaatatctcttgctaatgacggtacctgaagaataagcaccggctaactacgtgccagcagccgcggtaatacgtagggtgcaagcgttaatcggaattactgggcgtaaagcgtgcgcaggcggttttgtaagtctgatgtgaaatccccgggctcaacctgggaattgcattggagactgcaaggctagaatctggcagaggggggtagaattccacgtgtagcagtgaaatgcgtagatatgtggaggaacaccgatggcgaaggcagccccctgggtcaagattgacgctcatgcacgaaagcgtggggagcaaacaggattagataccctggtagtccacgccctaaacgatgtctactagttgtcgggtcttaattgacttggtaacgcagctaacgcgtgaagtagaccgcctggggagtacggtcgcaagattaaaactcaaaggaattgacggggacccgcacaagcggtggatgatgtggattaattcgatgcaacgcgaaaaaccttacctacccttgacatggctggaatccttgagagatcagggagtgctcgaaagagaaccagtacacaggtgctgcatggctgtcgtcagctcgtgtcgtgagatgttgggttaagtcccgcaacgagcgcaacccttgtcattagttgctacgaaagggcactctaatgagactgccggtgacaaaccggaggaaggtggggatgacgtcaagtcctcatggcccttatgggtagggcttcacacgtcatacaatggtacatacagagcgccgccaacccgcgagggggagctaatcgcagaaagtgtatcgtagtccggattgtagtctgcaactcgactgcatgaagttggaatcgctagtaatcgcggatcagcatgtcgcggtgaatacgttcccgggtcttgtacacaccgcccgtcacaccatgggagcgggttttaccagaagtaggtagcttaaccgtaaggagggcgcttaccacggtaggattcgtgactggggtgaagtcgtaacaaggtagccgtatcggaaggtgcggctggatcacctccttt

SEQ ID NO: 44 - RNA, 1497 nt, unidentified
  source 1..1497: mol_type = rRNA; note = DP44 16S rRNA sequence
tggcggcatgccttacacatgcaagtcgaacggcagcataggagcttgctcctgatggcgagtggcgaacgggtgagtaatatatcggaacgtgccctagagtgggggataactagtcgaaagactagctaataccgcatacgatctacggatgaaagtgggggatcgcaagacctcatgctcctggagcggccgatatctgattagctagttggtggggtaaaagctcaccaaggcgacgatcagtagctggtctgagaggacgaccagccacactgggactgagacacggcccagactcctacgggaggcagcagtggggaattttggacaatgggggcaaccctgatccagcaatgccgcgtgagtgaagaaggccttcgggttgtaaagctcttttgtcagggaagaaacggttctggataatacctaggactaatgacggtacctgaagaataagcaccggctaactacgtgccagcagccgcggtaatacgtagggtgcaagcgttaatcggaattactgggcgtaaagcgtgcgcaggcggttgtgtaagtcagatgtgaaatccccgggctcaacctgggaattgcatttgagactgcacggctagagtgtgtcagaggggggtagaattccacgtgtagcagtgaaatgcgtagatatgtggaggaataccgatggcgaaggcagccccctgggataacactgacgctcatgcacgaaagcgtggggagcaaacaggattagataccctggtagtccacgccctaaacgatgtctactagttgtcgggtcttaattgacttggtaacgcagctaacgcgtgaagtagaccgcctggggagtacggtcgcaagattaaaactcaaaggaattgacggggacccgcacaagcggtggatgatgtggattaattcgatgcaacgcgaaaaaccttacctacccttgacatggatggaatcccgaagagatttgggagtgctcgaaagagaaccatcacacaggtgctgcatggctgtcgtcagctcgtgtcgtgagatgttgggttaagtcccgcaacgagcgcaacccttgtcattagttgctacgaaagggcactctaatgagactgccggtgacaaaccggaggaaggtggggatgacgtcaagtcctcatggcccttatgggtagggcttcacacgtcatacaatggtacatacagagggccgccaacccgcgagggggagctaatcccagaaagtgtatcgtagtccggattggagtctgcaactcgactccatgaagttggaatcgctagtaatcgcggatcagcatgtcgcggtgaatacgttcccgggtcttgtacacaccgcccgtcacaccatgggagcgggttttaccagaagtgggtagcctaaccgcaaggagggcgctcaccacggtaggattcgtgactggggtgaagtcgtaacaaggtagccgtatcggaaggtgcggctggatcacctccttt

SEQ ID NO: 45 - RNA, 1522 nt, unidentified
  misc_feature 1..1522: note = DP45 16S rRNA sequence
  source 1..1522: mol_type = rRNA
tacggagagtttgatcctggctcaggacgaacgctggcggcgtgcttaacacatgcaagtcgaacggtgacgctagagcttgctctggttgatcagtggcgaacgggtgagtaacacgtgagtaacctgcccttgactctgggataactccgggaaaccggggctaataccggatacgagacgcgaccgcatggtcggcgtctggaaagtttttcggtcaaggatggactcgcggcctatcagcttgttggtgaggtaatggctcaccaaggcgtcgacgggtagccggcctgagagggcgaccggccacactgggactgagacacggcccagactcctacgggaggcagcagtggggaatattgcacaatgggcgaaagcctgatgcagcgacgccgcgtgagggatgaaggccttcgggttgtaaacctctttcagtagggaagaagcgaaagtgacggtacctgcagaagaagcgccggctaactacgtgccagcagccgcggtaatacgtagggcgcaagcgttgtccggaattattgggcgtaaagagctcgtaggcggtttgtcgcgtctggtgtgaaaactcaaggctcaaccttgagcttgcatcgggtacgggcagactagagtgtggtaggggtgactggaattcctggtgtagcggtggaatgcgcagatatcaggaggaacaccgatggcgaaggcaggtcactgggccactactgacgctgaggagcgaaagcatggggagcgaacaggattagataccctggtagtccatgccgtaaacgttgggcactaggtgtggggctcattccacgagttccgcgccgcagctaacgcattaagtgccccgcctggggagtacggccgcaaggctaaaactcaaaggaattgacgggggcccgcacaagcggcggagcatgcggattaattcgatgcaacgcgaagaaccttaccaaggcttgacatacaccggaatcatgcagagatgtgtgcgtcttcggactggtgtacaggtggtgcatggttgtcgtcagctcgtgtcgtgagatgttgggttaagtcccgcaacgagcgcaaccctcgtcctatgttgccagcacgttatggtggggactcataggagactgccggggtcaactcggaggaaggtggggatgacgtcaaatcatcatgccccttatgtcttgggcttcacgcatgctacaatggccggtacaaagggctgcgataccgcgaggtggagcgaatcccaaaaagccggtctcagttcggattggggtctgcaactcgaccccatgaagtcggagtcgctagtaatcgcagatcagcaacgctgcggtgaatacgttcccgggccttgtacacaccgcccgtcaagtcacgaaagtcggtaacacccgaagccggtggcctaaccccttgtgggatggagccgtcgaaggtgggattggcgattgggactaagtcgtaacaaggtagccgtaccggaaggtgcggctggatcacctccttt

SEQ ID NO: 46 - RNA, 831 nt, unidentified
  source 1..831: mol_type = rRNA; note = DP46 16S rRNA sequence
ttgaagagtttgatcatggctcagattgaacgctggcggcaggcctaacacatgcaagtcggacggtagcacagaggagcttgctccttgggtgacgagtggcggacgggtgagtaatgtctggggatctgcccgatagagggggataaccactggaaacggtggctaataccgcataacgtcgcaagaccaaagagggggaccttcgggcctctcactatcggatgaacccagatgggattagctagtaggcggggtaatggcccacctaggcgacgatccctagctggtctgagaggatgaccagccacactggaactgagacacggtccagactcctacgggaggcagcagtggggaatattgcacaatgggcgcaagcctgatgcagccatgccgcgtgtatgaagaaggccttcgggttgtaaagtactttcagcggggaggaaggcgacagggttaataaccctgtcgattgacgttacccgcagaagaagcaccggctaactccgtgccagcagccgcggtaatacggagggtgcaagcgttaatcggaattactgggcgtaaagcgcacgcaggcggtctgttaagtcagatgtgaaatccccgggcttaacctgggaactgcatttgaaactggcaggctttagtcttgtagagtggggtagaattccaggtgtagcggtgaaatgcgtagagatgtggaggaacaccagtggcgaaggcggctttttggtctgtaactgacgctgaggcgcgaaagcgtggggagcaaacaggattagataccctggtagtccacgccgtaaacgatgagtgctaagtgtt

SEQ ID NO: 47 - RNA, 960 nt, unidentified
  source 1..960: mol_type = rRNA; note = DP47 16S rRNA sequence
agggtgcaagcgttaatcggaattactgggcgtaaagcgcgcgtaggtggtttgttaagttgaatgtgaaatccccgggctcaacctgggaactgcatttgaaactggcaagctagagtctcgtagaggggggtagaattccaggtgtagcggtgaaatgcgtagagatctggaggaataccggtggcgaaggcggccccctggacgaagactgacgctcaggtgcgaaagcgtggggagcaaacaggattagataccctggtagtccacgccgtaaacgatgtcaactagccgttggaagccttgagcttttagtggcgcagctaacgcattaagttgaccgcctggggagtacggccgcaaggttaaaactcaaatgaattgacgggggcccgcacaagcggtggagcatgtggtttaattcgaagcaacgcgaagaaccttaccaggccttgacatccaatgaactttctagagatagattggtgccttcgggaacattgagacaggtgctgcatggctgtcgtcagctcgtgtcgtgagatgttgggttaagtcccgcaacgagcgcaacccttgtcctgtgttgccagcgcgtaatggcggggactcgcaggagactgccggggtcaactcggaggaaggtggggatgacgtcaaatcatcatgccccttatgtcttgggcttcacgcatgctacaatggccggtacaaagggctgcaataccgtgaggtggagcgaatcccaaaaagccggtcccagttcggattgaggtctgcaactcgacctcatgaagtcggagtcgctagtaatcgcagatcagcaacgctgcggtgaatacgttcccgggtcttgtacacaccgcccgtcaagtcatgaaagtcggtaacacctgaagccggtggcccaacccttgtggagggagccgtcgaaggtgggatcggtaattaggactaagt

SEQ ID NO: 48 - RNA, 1548 nt, unidentified
  source 1..1548: mol_type = rRNA; note = DP48 16S rRNA sequence
catggagagtttgatcctggctcaggacgaacgctggcggcgtgcctaatacatgcaagtcgagcggacagatgggagcttgctccctgatgttagcggcggacgggtgagtaacacgtgggtaacctgcctgtaagactgggataactccgggaaaccggggctaataccggatgcttgattgaaccgcatggttcaattataaaaggtggcttttagctaccacttacagatggacccgcggcgcattagctagttggtgaggtaacggctcaccaaggcaacgatgcgtagccgacctgagagggtgatcggccacactgggactgagacacggcccagactcctacgggaggcagcagtagggaatcttccgcaatggacgaaagtctgacggagcaacgccgcgtgagtgatgaaggttttcggatcgtaaaactctgttgttagggaagaacaagtaccgttcgaatagggcggtaccttgacggtacctaaccagaaagccacggctaactacgtgccagcagccgcggtaatacgtaggtggcaagcgttgtccggaattattgggcgtaaagcgcgcgcaggcggtttcttaagtctgatgtgaaagcccccggctcaaccggggagggtcattggaaactggggaacttgagtgcagaagaggagagtggaattccacgtgtagcggtgaaatgcgtagagatgtggaggaacaccagtggcgaaggcgactctctggtctgtaactgacgctgaggcgcgaaagcgtggggagcgaacaggattagataccctggtagtccacgccgtaaacgatgagtgctaagtgttagagggtttccgccctttagtgctgcagcaaacgcattaagcactccgcctggggagtacggtcgcaagactgaaactcaaaggaattgacgggggcccgcacaagcggtggagcatgtggtttaattcgaagcaacgcgaagaaccttaccaggtcttgacatcctctgacaaccctagagatagggcttccccttcgggggcagagtgacaggtggtgcatggttgtcgtcagctcgtgtcgtgagatgttgggttaagtcccgcaacgagcgcaacccttgatcttagttgccagcattcagttgggcactctaaggtgactgccggtgacaaaccggaggaaggtggggatgacgtcaaatcatcatgccccttatgacctgggctacacacgtgctacaatgggcagaacaaagggcagcgaagccgcgaggctaagccaatcccacaaatctgttctcagttcggatcgcagtctgcaactcgactgcgtgaagctggaatcgctagtaatcgcggatcagcatgccgcggtgaatacgttcccgggccttgtacacaccgcccgtcacaccacgagagtttgtaacacccgaagtcggtgaggtaaccttttggagccagccgccgaaggtgggacagatgattggggtgaagtcgtaacaaggtagccgtatcggaaggtgcggctggatcacctccttt

SEQ ID NO: 49 - RNA, 1551 nt, unidentified
  source 1..1551: mol_type = rRNA; note = DP49 16S rRNA sequence
tatggagagtttgatcctggctcaggacgaacgctggcggcgtgcctaatacatgcaagtcgagcggacgtttttgaagcttgcttcaaaaacgttagcggcggacgggtgagtaacacgtgggcaacctgccttatcgactgggataactccgggaaaccggggctaataccggataatatctagcacctcctggtgcaagattaaaagagggccttcgggctctcacggtgagatgggcccgcggcgcattagctagttggagaggtaatggctccccaaggcgacgatgcgtagccgacctgagagggtgatcggccacactgggactgagacacggcccagactcctacgggaggcagcagtagggaatcttccgcaatggacgaaagtctgacggagcaacgccgcgtgagtgatgaagggtttcggctcgtaaagctctgttatgagggaagaacacgtaccgttcgaatagggcggtaccttgacggtacctcatcagaaagccacggctaactacgtgccagcagccgcggtaatacgtaggtggcaagcgttgtccggaattattgggcgtaaagcgcgcgcaggcggccttttaagtctgatgtgaaatcttgcggctcaaccgcaagcggtcattggaaactgggaggcttgagtacagaagaggagagtggaattccacgtgtagcggtgaaatgcgtagatatgtggaggaacaccagtggcgaaggcgactctctggtctgtaactgacgctgaggcgcgaaagcgtggggagcaaacaggattagataccctggtagtccacgccgtaaacgatgagtgctaggtgttaggggtttcgatgcccgtagtgccgaagttaacacattaagcactccgcctggggagtacggccgcaaggctgaaactcaaaggaattgacgggggcccgcacaagcagtggagcatgtggtttaattcgaagcaacgcgaagaaccttaccaggtcttgacatcctttgaccactctggagacagagcttccccttcgggggcaaagtgacaggtggtgcatggttgtcgtcagctcgtgtcgtgagatgttgggttaagtcccgcaacgagcgcaacccttgaccttagttgccagcatttagttgggcactctaaggtgactgccggtgacaaaccggaggaaggtggggatgacgtcaaatcatcatgccccttatgacctgggctacacacgtgctacaatggatggtacaaagggttgcgaagccgcgaggtgaagccaatcccataaagccattctcagttcggattgtaggctgcaactcgcctgcatgaagctggaattgctagtaatcgcggatcagcatgccgcggtgaatacgttcccgggccttgtacacaccgcccgtcacaccacgagagtttgtaacacccgaagtcggtgaggtaaccttttggagccagccgccgaaggtgggacagatgattggggtgaagtcgtaacaaggtagccgtatcggaaggtgcggctggatcacctccttt

SEQ ID NO: 50 - RNA, 1536 nt, unidentified
  source 1..1536: mol_type = rRNA; note = DP50 16S rRNA sequence
ttgaagagtttgatcatggctcagattgaacgctggcggcaggcctaacacatgcaagtcgaacggtagcacagagagcttgctcttgggtgacgagtggcggacgggtgagtaatgtctgggaaactgcccgatggagggggataactactggaaacggtagctaataccgcataacgtcgcaagaccaaagtgggggaccttcgggcctcacaccatcggatgtgcccagatgggattagctagtaggtggggtaatggctcacctaggcgacgatccctagctggtctgagaggatgaccagccacactggaactgagacacggtccagactcctacgggaggcagcagtggggaatattgcacaatgggcgcaagcctgatgcagccatgccgcgtgtatgaagaaggccttcgggttgtaaagtactttcagcgaggaggaaggcattgtggttaataaccgcagtgattgacgttactcgcagaagaagcaccggctaactccgtgccagcagccgcggtaatacggagggtgcaagcgttaatcggaattactgggcgtaaagcgcacgcaggcggtctgtcaagtcggatgtgaaatccccgggctcaacctgggaactgcattcgaaactggcaggctagagtcttgtagaggggggtagaattccaggtgtagcggtgaaatgcgtagagatctggaggaataccggtggcgaaggcggccccctggacaaagactgacgctcaggtgcgaaagcgtggggagcaaacaggattagataccctggtagtccacgccgtaaacgatgtcgacttggaggttgtgcccttgaggcgtggcttccggagctaacgcgttaagtcgaccgcctggggagtacggccgcaaggttaaaactcaaatgaattgacgggggcccgcacaagcggtggagcatgtggtttaattcgatgcaacgcgaagaaccttacctactcttgacatccacggaatttagcagagatgctttagtgccttcgggaaccgtgagacaggtgctgcatggctgtcgtcagctcgtgttgtgaaatgttgggttaagtcccgcaacgagcgcaacccttatcctttgttgccagcggttcggccgggaactcaaaggagactgccagtgataaactggaggaaggtggggatgacgtcaagtcatcatggcccttacgagtagggctacacacgtgctacaatggcatatacaaagagaagcgacctcgcgagagcaagcggacctcataaagtatgtcgtagtccggatcggagtctgcaactcgactccgtgaagtcggaatcgctagtaatcgtagatcagaatgctacggtgaatacgttcccgggccttgtacacaccgcccgtcacaccatgggagtgggttgcaaaagaagtaggtagcttaaccttcgggagggcgcttaccactttgtgattcatgactggggtgaagtcgtaacaaggtaaccgtaggggaacctgcggttggatcacctcctt

SEQ ID NO: 51 - RNA, 1537 nt, unidentified
  source 1..1537: mol_type = rRNA; note = DP51 16S rRNA sequence
ttgaagagtttgatcatggctcagattgaacgctggcggcaggcctaacacatgcaagtcgagcggtagcacagggagcttgctcctgggtgacgagcggcggacgggtgagtaatgtctgggaaactgcctgatggagggggataactactggaaacggtagctaataccgcataacgtcgcaagaccaaagagggggaccttcgggcctcttgccatcagatgtgcccagatgggattagctagtaggtgaggtaatggctcacctaggcgacgatccctagctggtctgagaggatgaccagccacactggaactgagacacggtccagactcctacgggaggcagcagtggggaatattgcacaatgggcgcaagcctgatgcagccatgccgcgtgtatgaagaaggccttcgggttgtaaagtactttcagcgaggaggaaggcattaaggttaataaccttggtgattgacgttactcgcagaagaagcaccggctaactccgtgccagcagccgcggtaatacggggggtgcaagcgttaatcggaattactgggcgtaaagcgcacgcaggcggtttgtcaagtcggatgtgaaatccccgggctcaacctgggaactgcattcgaaacgggcaagctagagtcttgtagaggggggtagaattccaggtgtagcggtgaaatgcgtagagatctggaggaataccggtggcgaaggcggccccctggacaaagactgacgctcaggtgcgaaagcgtggggagcaaacaggattagataccctggtagtccacgccgtaaacgatgtcgacttggaggttgtgcccttgaggcgtggcttccggagctaacgcgttaagtcgaccgcctggggagtacggccgcaaggttaaaactcaaatgaattgacgggggcccgcacaagcggtggagcatgtggtttaattcgatgcaacgcgaagaaccttacctactcttgacatccagagaactttccagagatggattggtgccttcgggaactctgagacaggtgctgcatggctgtcgtcagctcgtgttgtgaaatgttgggttaagtcccgcaacgagcgcaacccttatcctttgttgccagcgagtaatgtcgggaactcaaaggagactgccagtgacaaactggaggaaggtggggatgacgtcaagtcatcatggcccttacgagtagggctacacacgtgctacaatggcatatacaaagagaagcgacctcgcgagagcaagcggacctcacaaagtatgtcgtagtccggatcggagtctgcaactcgactccgtgaagtcggaatcgctagtaatcgtagatcagaatgctacggtgaatacgttcccgggccttgtacacaccgcccgtcacaccatgggagtgggttgcaaaagaagtaggtagcttaaccttcgggagggcgcttaccactttgtgattcatgactggggtgaagtcgtaacaaggtaaccgtaggggaacctgcggttggatcacctcctt

SEQ ID NO: 52 - RNA, 1517 nt, unidentified
  source 1..1517: mol_type = rRNA; note = DP52 16S rRNA sequence
acggagagtttgatcctggctcaggatgaacgctggcggcgtgcttaacacatgcaagtcgaacgatgatcccagcttgctgggggattagtggcgaacgggtgagtaacacgtgagtaacctgcccttgactctgggataagcctgggaaactgggtctaataccggatatgactgtctgacgcatgtcaggtggtggaaagcttttgtggttttggatggactcgcggcctatcagcttgttggtggggtaatggcctaccaaggcgacgacgggtagccggcctgagagggtgaccggccacactgggactgagacacggcccagactcctacgggaggcagcagtggggaatattgcacaatgggcgcaagcctgatgcagcgacgccgcgtgagggatgacggccttcgggttgtaaacctctttcagtagggaagaagcgaaagtgacggtacctgcagaagaagcgccggctaactacgtgccagcagccgcggtaatacgtagggcgcaagcgttatccggaattattgggcgtaaagagctcgtaggcggtttgtcgcgtctgctgtgaaagaccggggctcaactccggttctgcagtgggtacgggcagactagagtgcagtaggggagactggaattcctggtgtagcggtgaaatgcgcagatatcaggaggaacaccgatggcgaaggcaggtctctgggctgtaactgacgctgaggagcgaaagcatggggagcgaacaggattagataccctggtagtccatgccgtaaacgttgggcactaggtgtgggggacattccacgttttccgcgccgtagctaacgcattaagtgccccgcctggggagtacggccgcaaggctaaaactcaaaggaattgacgggggcccgcacaagcggcggagcatgcggattaattcgatgcaacgcgaagaaccttaccaaggcttgacatgaaccggtaatacctggaaacaggtgccccgcttgcggtcggtttacaggtggtgcatggttgtcgtcagctcgtgtcgtgagatgttgggttaagtcccgcaacgagcgcaaccctcgttctatgttgccagcgcgttatggcggggactcataggagactgccggggtcaactcggaggaaggtggggacgacgtcaaatcatcatgccccttatgtcttgggcttcacgcatgctacaatggccggtacaaagggttgcgatactgtgaggtggagctaatcccaaaaagccggtctcagttcggattggggtctgcaactcgaccccatgaagtcggagtcgctagtaatcgcagatcagcaacgctgcggtgaatacgttcccgggccttgtacacaccgcccgtcaagtcacgaaagttggtaacacccgaagccggtggcctaacccttgtggggggagccgtcgaaggtgggaccggcgattgggactaagtcgtaacaaggtagccgtaccggaaggtgcggctggatcacctccttt

SEQ ID NO: 53 - RNA, 1406 nt, unidentified
  source 1..1406: mol_type = rRNA; note = DP53 16S rRNA sequence
tgaagagtttgatcatggctcagattgaacgctggcggcaggcctaacacatgcaagtcgagcggtagagagaagcttgcttctcttgagagcggcggacgggtgagtaatacctaggaatctgcctgatagtgggggataacgttcggaaacggacgctaataccgcatacgtcctacgggagaaagcaggggaccttcgggccttgcgctatcagatgagcctaggtcggattagctagttggtgaggtaatggctcaccaaggctacgatccgtaactggtctgagaggatgatcagtcacactggaactgagacacggtccagactcctacgggaggcagcagtggggaatattggacaatgggcgaaagcctgatccagccatgccgcgtgtgtgaagaaggtcttcggattgtaaagcactttaagttgggaggaagggcagttacctaatacgtgattgtcttgacgttaccgacagaataagcaccggctaactctgtgccagcagccgcggtaatacagagggtgcaagcgttaatcggaattactgggcgtaaagcgcgcgtaggtggtttgttaagttgaatgtgaaatccccgggctcaacctgggaactgcatccaaaactggcaagctagagtatggtagagggtagtggaatttcctgtgtagcggtgaaatgcgtagatataggaaggaacaccagtggcgaaggcgactacctggactgatactgacactgaggtgcgaaagcgtggggagcaaacaggattagataccctggtagtccacgccgtaaacgatgtcaactagccgttgggagtcttgaactcttagtggcgcagctaacgcattaagttgaccgcctggggagtacggccgcaaggttaaaactcaaatgaattgacgggggcccgcacaagcggtggagcatgtggtttaattcgaagcaacgcgaagaaccttaccaggccttgacatccaatgaactttctagagatagattggtgccttcgggaacattgagacaggtgctgcatggctgtcgtcagctcgtgtcgtgagatgttgggttaagtcccgtaacgagcgcaacccttgtccttagttaccagcacgtaatggtgggcactctaaggagactgccggtgacaaaccggaggaaggtggggatgacgtcaagtcatcatggcccttacggcctgggctacacacgtgctacaatggtcggtacaaagggttgccaagccgcgaggtggagctaatcccataaaaccgatcgtagtccggatcgcagtctgcaactcgactgcgtgaagtcggaatcgctagtaatcgtgaatcagaatgtcacggtgaatacgttcccgggccttgtacacaccgcccgtcacaccatg

SEQ ID NO: 54 - RNA, 1136 nt, unidentified
  source 1..1136: mol_type = rRNA; note = DP54 16S rRNA sequence
cttgagagtttgatcctggctcagagcgaacgctggcggcaggcttaacacatgcaagtcgagcgggcaccttcgggtgtcagcggcagacgggtgagtaacacgtgggaacgtacccttcggttcggaataacgctgggaaactagcgctaataccggatacgcccttttggggaaaggtttactgccgaaggatcggcccgcgtctgattagctagttggtggggtaacggcctaccaaggcgacgatcagtagctggtctgagaggatgatcagccacactgggactgagacacggcccagactcctacgggaggcagcagtggggaatattggacaatgggcgcaagcctgatccagccatgccgcgtgagtgatgaaggccttagggttgtaaagctcttttgtccgggacgataatgacggtaccggaagaataagccccggctaacttcgtgccagcagccgcggtaatacgaagggggctagcgttgctcggaatcactgggcgtaaagggcgcgtaggcggccattcaagtcgggggtgaaagcctgtggctcaaccacagaattgccttcgatactgtttggcttgagtttggtagaggttggtggaactgcgagtgtagaggtgaaattcgtagatattcgcaagaacaccagtggcgaaggcggccaactggaccaatactgacgctgaggcgcgaaagcgtggggagcaaacaggattagataccctggtagtccacgccgtaaacgatgaatgctagctgttggggtgcttgcacctcagtagcgcagctaacgctttaagcattccgcctggggagtacggtcgcaagattaaaactcaaaggaattgacgggggcccgcacaagcggtggagcatgtggtttaattcgaagcaacgcgcagaaccttaccatcccttgacatgtcgtgccatccggagagatccggggttcccttcggggacgcgaacacaggtgctgcatggctgtcgtcagctcgtgtcgtgagatgttgggttaagtcccgcaacgagcgcaacccacgtccttagttgccatcatttagttgggcactctagggagactgccggtgataagccgcgaggaaggtgtggatgacgtc

SEQ ID NO: 55 - RNA, 1374 nt, unidentified
  source 1..1374: mol_type = rRNA; note = DP55 16S rRNA sequence
tcggagagtttgatcctggctcaggatgaacgctggcggcgtgcctaatacatgcaagtcgagcgaactgattagaagcttgcttctatgacgttagcggcggacgggtgagtaacacgtgggcaacctgcctgtaagactgggataacttcgggaaaccgaagctaataccggataggatcttctccttcatgggagatgattgaaagatggtttcggctatcacttacagatgggcccgcggtgcattagctagttggtgaggtaacggctcaccaaggcaacgatgcatagccgacctgagagggtgatcggccacactgggactgagacacggcccagactcctacgggaggcagcagtagggaatcttccgcaatggacgaaagtctgacggagcaacgccgcgtgagtgatgaaggctttcgggtcgtaaaactctgttgttagggaagaacaagtacaagagtaactgcttgtaccttgacggtacctaaccagaaagccacggctaactacgtgccagcagccgcggtaatacgtaggtggcaagcgttatccggaattattgggcgtaaagcgcgcgcaggcggtttcttaagtctgatgtgaaagcccacggctcaaccgtggagggtcattggaaactggggaacttgagtgcagaagagaaaagcggaattccacgtgtagcggtgaaatgcgtagagatgtggaggaacaccagtggcgaaggcggctttttggtctgtaactgacgctgaggcgcgaaagcgtggggagcaaacaggattagataccctggtagtccacgccgtaaacgatgagtgctaagtgttagagggtttccgccctttagtgctgcagctaacgcattaagcactccgcctggggagtacggtcgcaagactgaaactcaaaggaattgacgggggcccgcacaagcggtggagcatgtggtttaattcgaagcaacgcgaagaaccttaccaggtcttgacatcctctgacaactctagagatagagcgttccccttcgggggacagagtgacaggtggtgcatggttgtcgtcagctcgtgtcgtgagatgttgggttaagtcccgcaacgagcgcaacccttgatcttagttgccagcatttagttgggcactctaaggtgactgccggtgacaaaccggaggaaggtggggatgacgtcaaatcatcatgccccttatgacctgggctacacacgtgctacaatggatggtacaaagggctgcaagaccgcgaggtcaagccaatcccataaaaccattctcagttcggattgtaggctgcaactcgcctacatgaagctggaatcgctagtaatcgcggatcagcatgct

SEQ ID NO: 56 - RNA, 1554 nt, unidentified
  source 1..1554: mol_type = rRNA; note = DP56 16S rRNA sequence
attggagagtttgatcctggctcaggacgaacgctggcggcgtgcctaatacatgcaagtcgagcggacctgatggagtgcttgcactcctgatggttagcggcggacgggtgagtaacacgtaggcaacctgccctcaagactgggataactaccggaaacggtagctaataccggataatttatttcacagcattgtggaataatgaaagacggagcaatctgtcacttggggatgggcctgcggcgcattagctagttggtggggtaacggctcaccaaggcgacgatgcgtagccgacctgagagggtgaacggccacactgggactgagacacggcccagactcctacgggaggcagcagtagggaatcttccgcaatgggcgaaagcctgacggagcaacgccgcgtgagtgatgaaggttttcggatcgtaaagctctgttgccaaggaagaacgtcttctagagtaactgctaggagagtgacggtacttgagaagaaagccccggctaactacgtgccagcagccgcggtaatacgtagggggcaagcgttgtccggaattattgggcgtaaagcgcgcgcaggcggttctttaagtctggtgtttaaacccgaggctcaacttcgggtcgcactggaaactggggaacttgagtgcagaagaggagagtggaattccacgtgtagcggtgaaatgcgtagatatgtggaggaacaccagtggcgaaggcgactctctgggctgtaactgacgctgaggcgcgaaagcgtggggagcaaacaggattagataccctggtagtccacgccgtaaacgatgaatgctaggtgttaggggtttcgatacccttggtgccgaagttaacacattaagcattccgcctggggagtacggtcgcaagactgaaactcaaaggaattgacggggacccgcacaagcagtggagtatgtggtttaattcgaagcaacgcgaagaaccttaccaagtcttgacatccctctgaatcctctagagatagaggcggccttcgggacagaggtgacaggtggtgcatggttgtcgtcagctcgtgtcgtgagatgttgggttaagtcccgcaacgagcgcaacccttgattttagttgccagcacatcatggtgggcactctagaatgactgccggtgacaaaccggaggaaggcggggatgacgtcaaatcatcatgccccttatgacttgggctacacacgtactacaatggctggtacaacgggaagcgaagccgcgaggtggagccaatcctataaaagccagtctcagttcggattgcaggctgcaactcgcctgcatgaagtcggaattgctagtaatcgcggatcagcatgccgcggtgaatacgttcccgggtcttgtacacaccgcccgtcacaccacgagagtttacaacacccgaagtcggtggggtaacccgcaagggagccagccgccgaaggtggggtagatgattggggtgaagtcgtaacaaggtagccgtatcggaaggtgcggctggatcacctccttt

SEQ ID NO: 57 - RNA, 1550 nt, unidentified
  source 1..1550: mol_type = rRNA; note = DP57 16S rRNA sequence
attggagagtttgatcctggctcaggatgaacgctggcggcgtgcctaatacatgcaagtcgagcgaatggattaagagcttgctcttatgaagttagcggcggacgggtgagtaacacgtgggtaacctgcccataagactgggataactccgggaaaccggggctaataccggataacattttgcaccgcatggtgcgaaattcaaaggcggcttcggctgtcacttatggatggacccgcgtcgcattagctagttggtgaggtaacggctcaccaaggcaacgatgcgtagccgacctgagagggtgatcggccacactgggactgagacacggcccagactcctacgggaggcagcagtagggaatcttccgcaatggacgaaagtctgacggagcaacgccgcgtgagtgatgaaggctttcgggtcgtaaaactctgttgttagggaagaacaagtgctagttgaataagctggcaccttgacggtacctaaccagaaagccacggctaactacgtgccagcagccgcggtaatacgtaggtggcaagcgttatccggaattattgggcgtaaagcgcgcgcaggtggtttcttaagtctgatgtgaaagcccacggctcaaccgtggagggtcattggaaactgggagacttgagtgcagaagaggaaagtggaattccatgtgtagcggtgaaatgcgtagagatatggaggaacaccagtggcgaaggcgactttctggtctgtaactgacactgaggcgcgaaagcgtggggagcaaacaggattagataccctggtagtccacgccgtaaacgatgagtgctaagtgttagagggtttccgccctttagtgctgaagttaacgcattaagcactccgcctggggagtacggccgcaaggctgaaactcaaaggaattgacgggggcccgcacaagcggtggagcatgtggtttaattcgaagcaacgcgaagaaccttaccaggtcttgacatcctctgacaaccctagagatagggcttccccttcgggggcagagtgacaggtggtgcatggttgtcgtcagctcgtgtcgtgagatgttgggttaagtcccgcaacgagcgcaacccttgatcttagttgccatcattaagttgggcactctaaggtgactgccggtgacaaaccggaggaaggtggggatgacgtcaaatcatcatgccccttatgacctgggctacacacgtgctacaatggacggtacaaagagctgcaagaccgcgaggtggagctaatctcataaaaccgttctcagttcggattgtaggctgcaactcgcctacatgaagctggaatcgctagtaatcgcggatcagcatgccgcggtgaatacgttcccgggccttgtacacaccgcccgtcacaccacgagagtttgtaacacccgaagtcggtggggtaacctttttggagccagccgcctaaggtgggacagatgattggggtgaagtcgtaacaaggtagccgtatcggaaggtgcggctggatcacctccttt

SEQ ID NO: 58 - RNA, 1060 nt, unidentified
  source 1..1060: mol_type = rRNA; note = DP58 16S rRNA sequence
aatgacggtacctgaagaataagcaccggctaactacgtgccagcagccgcggtaatacgtagggtgcaagcgttaatcggaattactgggcgtaaagcgtgcgcaggcggttttgtaagtctgatgtgaaatccccgggctcaacctgggaattgcattggagactgcaaggctagaatctggcagaggggggtagaattccacgtgtagcagtgaaatgcgtagatatgtggaggaacaccgatggcgaaggcagccccctgggtcaagattgacgctcatgcacgaaagcgtggggagcaaacaggattagataccctggtagtccacgccctaaacgatgtctactagttgtcgggtcttaattgacttggtaacgcagctaacgcgtgaagtagaccgcctggggagtacggtcgcaagattaaaactcaaaggaattgacggggacccgcacaagcggtggatgatgtggattaattcgatgcaacgcgaaaaaccttacctacccttgacatggctggaatcctcgagagattggggagtgctcgaaagagaaccagtacacaggtgctgcatggctgtcgtcagctcgtgtcgtgagatgttgggttaagtcccgcaacgagcgcaacccttgtcattagttgctacgaaagggcactctaatgagactgccggtgacaaaccggaggaaggtggggatgacgtcaagtcctcatggcccttatgggtagggcttcacacgtcatacaatggtacatacagagcgccgccaacccgcgagggggagctaatcgcagaaagtgtatcgtagtccggattgtagtctgcaactcgactgcatgaagttggaatcgctagtaatcgcggatcagcatgtcgcggtgaatacgttcccgggtcttgtacacaccgcccgtcacaccatgggagcgggttttaccagaagtaggtagcttaaccgtaaggagggcgcttaccacggtaggattcgtgactggggtgaagtcgtaacaaggtagccgtatcggaaggtgcggctggatcacctccttt

SEQ ID NO: 59 - RNA, 1538 nt, unidentified
  source 1..1538: mol_type = rRNA; note = DP59 16S rRNA sequence
ttgaagagtttgatcatggctcagattgaacgctggcggcaggcctaacacatgcaagtcgaacggtaacaggaagcagcttgctgctttgctgacgagtggcggacgggtgagtaatgtctgggaaactgcctgatggagggggataactactggaaacggtagctaataccgcataacgtcgcaagaccaaagagggggaccttcgggcctcttgccatcagatgtgcccagatgggattagctagtaggtggggtaacggctcacctaggcgacgatccctagctggtctgagaggatgaccagccacactggaactgagacacggtccagactcctacgggaggcagcagtggggaatattgcacaatgggcgcaagcctgatgcagccatgccgcgtgtatgaagaaggccttcgggttgtaaagtactttcagcggggaggaaggcgatgcggttaataaccgcgtcgattgacgttacccgcagaagaagcaccggctaactccgtgccagcagccgcggtaatacggagggtgcaagcgttaatcggaattactgggcgtaaagcgcacgcaggcggtctgtcaagtcggatgtgaaatccccgggctcaacctgggaactgcatccgaaactggcaggcttgagtctcgtagaggggggtagaattccaggtgtagcggtgaaatgcgtagagatctggaggaataccggtggcgaaggcggccccctggacgaagactgacgctcaggtgcgaaagcgtggggagcaaacaggattagataccctggtagtccacgccgtaaacgatgtcgacttggaggttgtgcccttgaggcgtggcttccggagctaacgcgttaagtcgaccgcctggggagtacggccgcaaggttaaaactcaaatgaattgacgggggcccgcacaagcggtggagcatgtggtttaattcgatgcaacgcgaagaaccttacctggtcttgacatccacagaacttggcagagatgccttggtgccttcgggaactgtgagacaggtgctgcatggctgtcgtcagctcgtgttgtgaaatgttgggttaagtcccgcaacgagcgcaacccttatcctttgttgccagcggttaggccgggaactcaaaggagactgccagtgataaactggaggaaggtggggatgacgtcaagtcatcatggcccttacgaccagggctacacacgtgctacaatggcgcatacaaagagaagcgatctcgcgagagccagcggacctcataaagtgcgtcgtagtccggattggagtctgcaactcgactccatgaagtcggaatcgctagtaatcgtgaatcagaatgtcacggtgaatacgttcccgggccttgtacacaccgcccgtcacaccatgggagtgggttgcaaaagaagtaggtagcttaaccttcgggagggcgcttaccactttgtgattcatgactggggtgaagtcgtaacaaggtaaccgtaggggaacctgcggttggatcacctcctt

SEQ ID NO: 60 - RNA, 1547 nt, unidentified
  source 1..1547: mol_type = rRNA; note = DP60 16S rRNA sequence
tcggagagtttgatcctggctcaggacgaacgctggcggcgtgcctaatacatgcaagtcgagcgaatcgatgggagcttgctccctgagattagcggcggacgggtgagtaacacgtgggcaacctgcctataagactgggataacttcgggaaaccggagctaataccggatacgttcttttctcgcatgagagaagatggaaagacggttttgctgtcacttatagatgggcccgcggcgcattagctagttggtgaggtaatggctcaccaaggcgacgatgcgtagccgacctgagagggtgatcggccacactgggactgagacacggcccagactcctacgggaggcagcagtagggaatcttccgcaatggacgaaagtctgacggagcaacgccgcgtgaacgaagaaggccttcgggtcgtaaagttctgttgttagggaagaacaagtaccagagtaactgctggtaccttgacggtacctaaccagaaagccacggctaactacgtgccagcagccgcggtaatacgtaggtggcaagcgttgtccggaattattgggcgtaaagcgcgcgcaggtggttccttaagtctgatgtgaaagcccacggctcaaccgtggagggtcattggaaactggggaacttgagtgcagaagaggaaagtggaattccaagtgtagcggtgaaatgcgtagagatttggaggaacaccagtggcgaaggcgactttctggtctgtaactgacactgaggcgcgaaagcgtggggagcaaacaggattagataccctggtagtccacgccgtaaacgatgagtgctaagtgttagagggtttccgccctttagtgctgcagctaacgcattaagcactccgcctggggagtacggccgcaaggctgaaactcaaaggaattgacgggggcccgcacaagcggtggagcatgtggtttaattcgaagcaacgcgaagaaccttaccaggtcttgacatcctctgacaaccctagagatagggcgttccccttcgggggacagagtgacaggtggtgcatggttgtcgtcagctcgtgtcgtgagatgttgggttaagtcccgcaacgagcgcaacccttgatcttagttgccagcattcagttgggcactctaaggtgactgccggtgacaaaccggaggaaggtggggatgacgtcaaatcatcatgccccttatgacctgggctacacacgtgctacaatggatggtacaaagggctgcaaacctgcgaaggtaagcgaatcccataaagccattctcagttcggattgtaggctgcaactcgcctacatgaagccggaatcgctagtaatcgcggatcagcatgccgcggtgaatacgttcccgggccttgtacacaccgcccgtcacaccacgagagtttgtaacacccgaagtcggtgaggtaacctttatggagccagccgcctaaggtgggacagatgattggggtgaagtcgtaacaaggtagccgtatcggaaggtgcggctggatcacctccttt

SEQ ID NO: 61 - RNA, 962 nt, unidentified
  source 1..962: mol_type = rRNA; note = DP61 16S rRNA sequence
ggaaggcggtctgtcaagtcggatgtgaaatccccgggctcaacctgggaactgcattcgaaactggcaggctagagtcttgtagaggggggtagaattccaggtgtagcggtgaaatgcgtagagatctggaggaataccggtggcgaaggcggccccctggacaaagactgacgctcaggtgcgaaagcgtggggagcaaacaggattagataccctggtagtccacgccgtaaacgatgtcgacttggaggttgttcccttgaggagtggcttccggagctaacgcgttaagtcgaccgcctggggagtacggccgcaaggttaaaactcaaatgaattgacgggggcccgcacaagcggtggagcatgtggtttaattcgatgcaacgcgaagaaccttacctactcttgacatccacggaatttagcagagatgctttagtgccttcgggaaccgtgagacaggtgctgcatggctgtcgtcagctcgtgttgtgaaatgttgggttaagtcccgcaacgagcgcaacccttatcctttgttgccagcggtccggccgggaactcaaaggagactgccagtgataaactggaggaaggtggggatgacgtcaagtcatcatggcccttacgagtagggctacacacgtgctacaatggcgcatacaaagagaagcgacctcgcgagagcaagcggacctcataaagtgcgtcgtagtccggatcggagtctgcaactcgactccgtgaagtcggaatcgctagtaatcgtagatcagaatgctacggtgaatacgttcccgggccttgtacacaccgcccgtcacaccatgggagtgggttgcaaaagaagtaggtagcttaaccttcgggagggcgcttaccactttgtgattcatgactggggtgaagtcgtaacaaggtaaccgtaggggaacctgcggttggatcacctcctt

SEQ ID NO: 62 - RNA, 876 nt, unidentified
  source 1..876: mol_type = rRNA; note = DP62 16S rRNA sequence
tggctcagattgaacgctggcggcaggcctaacacatgcaagtcgaacggtagcacagaggagcttgctccttgggtgacgagtggcggacgggtgagtaatgtctgggaaactgcccgatggagggggataactactggaaacggtagctaataccgcataacgtcttcggaccaaagtgggggaccttcgggcctcacaccatcggatgtgcccagatgggattagctagtaggtggggtaatggctcacctaggcgacgatccctagctggtctgagaggatgaccagccacactggaactgagacacggtccagactcctacgggaggcagcagtggggaatattgcacaatgggcgcaagcctgatgcagccatgccgcgtgtatgaagaaggccttcgggttgtaaagtactttcagtggggaggaaggcgttaaggttaataaccttggcgattgacgttacccgcagaagaagcaccggctaactccgtgccagcagccgcggtaatacggagggtgcaagcgttaatcggaattactgggcgtaaagcgcacgcaggcggtctgtcaagtcggatgtgaaatccccgggctcaacctgggaactgcattcgaaactggcaggctagagtcttgtagaggggggtagaattccaggtgtagcggtgaaatgcgtagagatctggaggaataccggtggcgaaggcggccccctggacaaagactgacgctcaggtgcgaaagcgtggggagcaaacaggattagataccctggtagtccacgccgtaaacgatgtcgacttggaggttgttcccttgaggagtggcttccggagctaacgcgttaagtcgaccgcctggggagtacgg

SEQ ID NO: 63 - RNA, 1532 nt, unidentified
  source 1..1532: mol_type = rRNA; note = DP63 16S rRNA sequence
tgaagagtttgatcatggctcagattgaacgctggcggcaggcctaacacatgcaagtcgagcggtagagagaagcttgcttctcttgagagcggcggacgggtgagtaatgcctaggaatctgcctggtagtgggggataacgttcggaaacggacgctaataccgcatacgtcctacgggagaaagcaggggaccttcgggccttgcgctatcagatgagcctaggtcggattagctagttggtgaggtaatggctcaccaaggcgacgatccgtaactggtctgagaggatgatcagtcacactggaactgagacacggtccagactcctacgggaggcagcagtggggaatattggacaatgggcgaaagcctgatccagccatgccgcgtgtgtgaagaaggtcttcggattgtaaagcactttaagttgggaggaagggttgtagattaatactctgcaattttgacgttaccgacagaataagcaccggctaactctgtgccagcagccgcggtaatacagagggtgcaagcgttaatcggaattactgggcgtaaagcgcgcgtaggtggtttgttaagttggatgtgaaatccccgggctcaacctgggaactgcattcaaaactgactgactagagtatggtagagggtggtggaatttcctgtgtagcggtgaaatgcgtagatataggaaggaacaccagtggcgaaggcgaccacctggactaatactgacactgaggtgcgaaagcgtggggagcaaacaggattagataccctggtagtccacgccgtaaacgatgtcaactagccgttggaagccttgagcttttagtggcgcagctaacgcattaagttgaccgcctggggagtacggccgcaaggttaaaactcaaatgaattgacgggggcccgcacaagcggtggagcatgtggtttaattcgaagcaacgcgaagaaccttaccaggccttgacatccaatgaactttctagagatagattggtgccttcgggaacattgagacaggtgctgcatggctgtcgtcagctcgtgtcgtgagatgttgggttaagtcccgtaacgagcgcaacccttgttcttagttaccagcacgttatggtgggcactctaaggagactgccggtgacaaaccggaggaaggtggggatgacgtcaagtcatcatggcccttacggcctgggctacacacgtgctacaatggtcggtacagagggttgccaagccgcgaggtggagctaatcccataaaaccgatcgtagtccggatcgcagtctgcaactcgactgcgtgaagtcggaatcgctagtaatcgcgaatcagaatgtcgcggtgaatacgttcccgggccttgtacacaccgcccgtcacaccatgggagtgggttgcaccagaagtagctagtctaaccttcgggaggacggttaccacggtgtgattcatgactggggtgaagtcgtaacaaggtagccgtaggggaacctgcggctggatcacctcctt

SEQ ID NO: 64 - DNA, 1649 bp, unidentified
  source 1..1649: mol_type = genomic DNA; note = DP64 ITS sequence
tccgtaggtgaacctgcggaaggatcattaaataatcaataattttggcttgtccattattatctatttactgtgaactgtattattacttgacgcttgagggatgctccactgctataaggataggcggtggggatgttaaccgagtcatagtcaagcttaggcttggtatcctattattatttaccaaaagaattcagaattaatattgtaacatagacctaaaaaatctataaaacaacttttaacaacggatctcttggttctcgcatcgatgaagaacgtagcaaagtgcgataactagtgtgaattgcatattcagtgaatcatcgagtctttgaacgcaacttgcgctcattggtattccaatgagcacgcctgtttcagtatcaaaacaaaccctctattcaatatttttgttgaataggaatactgagagtctcttgatcttttctgatctcgaacctcttgaaatgtacaaaggcctgatcttgtttgaatgcctgaacttttttttaatataaagagaagctcttgcggtaaactgtgctggggcctcccaaataatactctttttaaatttgatctgaaatcaggcgggattacccgctgaacttaagcatatcaataagcggaggaaaagaaaataacaatgatttccctagtaacggcgagtgaagaggaaagagctcaaagttggaaactgtttggcttagctaaaccgtattgtaaactgtagaaacattttcctggcacgccggattaataagtcctttggaacaaggcatcatggagggtgagaatcccgtctttgatccgagtagttgtcttttgtgatatgttttcaaagagtcaggttgtttgggaatgcagcctaaattgggtggtaaatctcacctaaagctaaatatttgcgagagaccgatagcgaacaagtaccgtgagggaaagatgaaaagaactttgaaaagagagttaaacagtatgtgaaattgttaaaagggaaccgtttggagccagactggtttgactgtaatcaacctagaattcgttctgggtgcacttgcagtctatacctgccaacaacagtttgatttggaggaaaaaattagtaggaatgtagcctctcgaggtgttatagcctactatcatactctggattggactgaggaacgcagcgaatgccattaggcgagattgctgggtgctttcgctaataaatgttagaatttctgcttcgggtggtgctaatgtttaaaggaggaacacatctagtatattttttattcgcttaggttgttggcttaatgactctaaatgacccgtcttgaaacacggaccaaggagtccaccataagtgcaagtatttgagtgacaaactcatatgcgtaaggaaactgattgatacgaaatcttttgatggcagtatcacccggcgttgacgttttatactgaactgaccgaggtaaagcacttatgatgggacccgaaagatggtgaactatgcctgaatagggtgaagccagaggaaactctggtggaggctcgtagcgattctgacgtgcaaatcgatcgtcaaatttgggtataggggcgaaagactaatcgaaccatctagtagctggttcctgccgaagtttccctcagga

SEQ ID NO: 65 - DNA, 1498 bp, unidentified
  source 1..1498: mol_type = genomic DNA; note = DP65 ITS sequence
tccgtaggtgaacctgcggaaggatcattattgaaaacaagggtgtccaatttaacttggaacccgaacttctcaattctaactttgtgcatctgtattatggcgagcagtcttcggattgtgagccttcacttataaacactagtctatgaatgtaaaatttttataacaaataaaaactttcaacaacggatctcttggctctcgcatcgatgaagaacgcagcgaaatgcgatacgtaatgtgaattgcagaattcagtgaatcatcgaatctttgaacgcatcttgcgctctctggtattccggagagcatgtctgtttgagtgtcatgaattcttcaacccaatcttttcttgtaatcgattggtgtttggattttgagcgctgctggcttcggcctagctcgttcgtaatacattagcatccctaatacaagtttggattgacttggcgtaatagactattcgctaaggattcggtggaaacatcgagccaacttcattaaggaagctcctaatttaaaagtctaccttttgattagatctcaaatcaggcaggattacccgctgaacttaagcatatcaataagcggaggaaaagaaactaacaaggattcccctagtagcggcgagcgaagcgggaaaagctcaaatttgtaatctggcgtcttcgacgtccgagttgtaatctcgagaagtgttttccgtgatagaccgcatacaagtctcttggaacagagcgtcatagtggtgagaacccagtacacgatgcggatgcctattactttgtgatacactttcgaagagtcgagttgtttgggaatgcagctcaaattgggtggtaaattccatctaaagctaaatattggcgagagaccgatagcgaacaagtaccgtaagggaaagatgaaaagcactttggaaagagagttaacagtacgtgaaattgttggaagggaaacacatgcagtgatacttgctattcggggcaactcgattggcaggcccgcatcagtttttcggggcggaaaagcgtagagagaaggtagcaatttcggttgtgttatagctctttactggattcgccctgggggactgaggaacgcagcgtgcttttagcaattccttcgggaattccacgcttaggatgcgggtttatggctgtatatgacccgtcttgaaacacggaccaaggagtctaacatgcttgcgagtatttgggtgtcaaacccggatgcgcaatgaaagtgaatggaggtgggaagcgcaagctgcaccatcgaccgatctggattttttaagatggatttgagtaagagcaagtatgttgggacccgaaagatggtgaactatgcctgaatagggcgaagccagaggaaactctggtggaggctcgtagcggttctgacgtgcaaatcgatcgtcaaatttgggtataggggcgaaagactaatcgaaccatctagtagctggttcctgccgaagtttccctcagga

SEQ ID NO: 66 - DNA, 1340 bp, unidentified
  source 1..1340: mol_type = genomic DNA; note = DP66 ITS sequence
tccgtaggtgaacctgcggaaggatcattactgtgatttatccaccacactgcgtgggcgacacgaaacaccgaaaccgaacgcacgccgtcaagcaagaaatccacaaaactttcaacaacggatctcttggttctcgcatcgatgaagagcgcagcgaaatgcgatacctagtgtgaattgcagccatcgtgaatcatcgagttcttgaacgcacattgcgcccgctggtattccggcgggcatgcctgtctgagcgtcgtttccttcttggagcggagcttcagacctggcgggctgtctttcgggacggcgcgcccaaagcgaggggccttctgcgcgaactagactgtgcgcgcggggcggccggcgaacttataccaagctcgacctcagatcaggcaggagtacccgctgaacttaagcatatcaataagcggaggaaaagaaaccaacagggattgccccagtagcggcgagtgaagcggcaaaagctcagatttggaatcgcttcggcgagttgtgaattgcaggttggcgcctctgcggcggcggcggtccaagtcccttggaacagggcgccattgagggtgagagccccgtgggaccgtttgcctatgctctgaggcccttctgacgagtcgagttgtttgggaatgcagctctaagcgggtggtaaattccatctaaggctaaatactggcgagagaccgatagcgaacaagtactgtgaaggaaagatgaaaagcactttgaaaagagagtgaaacagcacgtgaaattgttgaaagggaagggtattgcgcccgacatggagcgtgcgcaccgctgcccctcgtgggcggcgctctgggcgtgctctgggccagcatcggtttttgccgcgggagaagggcggcgggcatgtagctcttcggagtgttatagcctgccgccggcgccgcgagcggggaccgaggactgcgacttttgtctcggatgctggcacaacggcgcaacaccgcccgtcttgaaacatggaccaaggagtctaacgtctatgcgagtgtttgggtgtgaaaccccgggcgcgtaatgaaagtgaacgtaggtcggaccgctcctctcggggggcgggcacgatcgaccgatcctgatgtcttcggatggatttgagtaagagcatagctgttgggacccgaaagatggtgaactatgcctgaatagggtgaagccagaggaaactctggtggaggctcgtagcggttctgacgtgcaaatcgatcgtcgaatttgggtataggggcgaaagactaatcgaaccatctagtagctggttcctgccgaagtttccctcagga

SEQ ID NO: 67 - DNA, 1698 bp, unidentified
  source 1..1698: mol_type = genomic DNA; note = DP53 Glutamine--tRNA ligase sequence
atgagcaagcccactgtcgaccccactctgaatccaaaggctggccctgctgtcccggctaacttcctgcgtccaatcgttcaggcggacctagactcgggtaaatacacacagatcgtgacccgctttccgccggagccaaacggctatctgcacatcggtcatgccaaatccatttgtgtgaactttgggctggctcaagagtttggcggcgtgacgcatttgcgttttgacgacaccaacccggcaaaagaagaccaggaatacatcgacgccatcgaaagcgacgtcaagtggctgggcttcgagtgggccggtgaagtgcgttacgcgtcgcaatacttcgatcaactgcacgagtgggcgatttacctgatcaaagaaggcaaggcctacgtctgcgacctgacgcccgagcaagccaaggaataccgtggcagcctgaccgagcccggcaagaacagcccgttccgcgaccgtagcgttgaagagaacctggatctgttcgcccgcatgaccgccggtgagtttgaagacggcaagcgtgtgctgcgcgccaagatcgacatgacctcgccgaacatgaacctgcgcgacccgatcatgtaccgcatccgtcatgcccatcaccaccagaccggtgacaagtggtgcatctaccccaactatgacttcacccacggtcagtcggatgccattgaaggcatcacccattcgatctgcaccctggagttcgaaagccatcgtccgctgtacgaatggttcctggacagcctgccagtaccggcgcgcccgcgtcagtacgagttcagccgtctgaacctcaactacaccatcaccagcaagcgcaagctcaagcagctggtcgatgaaaagcacgtcaacggctgggatgacccgcgcatgtcgacgctgtcgggtttccgccgtcgcggttacacgcctaaatcgattcgtaatttctgtgacatggtcggcaccaaccgttctgacggtgttgttgacttcggcatgctggaattcagcattcgtgacgatttggaccacagcgcgccgcgcgccatgtgcgtgctgcgtccattgaaggtgattattaccaactacccggaaggtcaggtcgaaaacctcgagctgccttgccacccgaaagaagacatgggtgtgcgggtgttgccgtttgcccgtgaaatctacatcgaccgtgaagacttcatggaagagccgccaaaaggctacaagcgtcttgagcctgcgggcgaagtgcgtttgcgcggcagctatgtgatccgtgccgacgaagcgatcaaggatgccgatggcaacatcgttgaactgcattgctcgtacgatccgctgaccctgggtaaaaaccctgaaggtcgcaaggtcaagggtgttgtgcactgggtgccggcggcggccagcgtcgaatgcgaagtgcgtttgtatgatcgtctgttccgctcgccgaaccctgaaaaggccgaagacggcgcgggcttcctggaaaacatcaaccctgactcgctgcaggtactgaccggttgtcgtgctgaaccctcgctgggcaatgcacagccggaagaccgtttccagttcgagcgcgaaggctacttctgcgcagatatcaaggactcgaaacccggtcacccggtattcaaccgtaccgtgaccctgcgtgattcgtggggccagtga

SEQ ID NO: 68 - DNA, 2418 bp, unidentified
  source 1..2418: mol_type = genomic DNA; note = DP53 DNA gyrase subunit B sequence
ttgagcgaagaaaacacgtacgactcaacgagcattaaagtgctgaaaggccttgatgccgtacgcaaacgtcccggtatgtacattggtgatactgacgatggcagcggtctgcaccacatggtgttcgaagtagtcgacaactccatcgacgaagcgctggctggccattgcgacgacatcaccatcacgatccacccggacgagtccatcaccgtgcgcgataacggccgcggtattccggttgacgtgcataaagaagaaggcgtatctgcagccgaggtcatcatgaccgtgctgcacgccggcggtaagttcgatgacaactcctacaaagtatccggcggcttgcacggtgtaggtgtttcggtggtaaacgccctgtccgaactgctggtcttgactgtacgccgcagcggcaagatctgggaacagacctacgtccacggtgttcctcaggcgcctatggctattgtgggtgaaagcgaaaccacgggtacgcagatccacttcaagccttcggctgaaaccttcaagaatatccactttagctgggacatcctggccaagcggattcgtgaactgtccttcctgaactccggtgtgggtatcgtcctcaaggacgagcgcagcggcaaggaggagctgttcaagtacgaaggtggcctgcgtgcattcgttgattacctgaacaccaacaagaacgctgtgaaccaggtgttccacttcaatgttcagcgtgaagacggcatcggcgtagaaatcgccctgcagtggaacgacagcttcaacgagaacctgttgtgcttcaccaacaacattccacagcgcgatggtggcacgcacttggtgggcttccgctctgccctgacgcgtaacctcaacacgtacatcgaagctgaaggcctggccaagaagcacaaggtcgccaccaccggtgatgacgcccgtgaaggcttgaccgcgatcatctcggtgaaagtgccggatccaaagttcagctcgcagactaaagacaagctggtgtcttccgaagtgaagaccgctgttgaacaggaaatgggcaagttcttctccgacttcctgctggaacacccgaacgaagccaagttgattgtcggcaagatgatcgacgcagcccgtgctcgtgaagctgcacgtaaagcccgtgagatgacccgtcgtaaaggcgcgttggacatcgcgggcttgccgggcaagctggctgactgccaggaaaaagaccctgctctgtccgaactgtacctggtggaaggtgactctgctggcggctccgccaagcagggtcgcaaccgtcgtacccaagccatcctgccgttgaaaggtaaaatcctcaacgtcgagaaagcccgttttgacaagatgatctcttcgcaagaagtcggcaccttgatcactgcgctgggctgtggcatcggccgcgaagagtacaacatcgacaaactgcgctatcacaacatcatcatcatgaccgatgctgacgttgacggttcgcacatccgtaccctgctgctgaccttcttcttccgtcagttgccggagctgatcgagcgtggctacatctacatcgcccagccaccgttgtacaaagtgaaaaagggcaagcaagagcagtacatcaaagacgacgaggccatggaagagtacatgacccagtcggctcttgaagatgccagcctgcacttgaacgaagatgcccctggcatctccggtgaggcactggagcgtctggtgtacgacttccgcatggtgatgaagaccctcaagcgtttgtcgcgcctgtaccctcaggagctgaccgagcacttcatctacctgccggctgtaagccttgagcagttgggtgaccacgctgccatgcaggactggatggccaagtttgaagagcgtctgcgtctggttgagaaatcgggcctggtctacaaagccagcctgcgtgaagaccgtgagcgtaatgtctggttgccagaggtcgaactgatctcccacggccactcgacgttcatcaccttcaaccgcgacttcttcggcagcaacgattacaaaaccgttgtgaccctgggcgctcaactgagcaccctgctggatgaaggcgcctatatccagcgtggcgaacgtcgcaagcaagtgaccgagttcaaagaagcactggactggttgatggctgaaagcaccaagcgtcacaccatccagcgctacaaaggactgggtgaaatgaacccggatcagctctgggaaaccacgatggacccaagcgtgcgtcgcatgctgaaagtcaccatcgaagacgcgatcggcgccgatcagatcttcaacaccttgatgggcgatgctgtagaaccacgtcgtgaattcatcgagagcaacgcactggcagtgtccaacctggatttctga

SEQ ID NO: 69 - DNA, 2832 bp, unidentified
  source 1..2832: mol_type = genomic DNA; note = DP53 Isoleucine--tRNA ligase sequence
atgaccgactacaaagccacgctaaacctcccggacaccgccttcccaatgaaggccggcctgccacagcgcgaaccgcaaattttgcagcgctgggacagcattggcctgtacgggaagttgcgcgagattggcaaggatcgtccgaagttcgtacttcacgacggtcctccgtacgccaacggcactatccatatcggtcatgcgctgaacaagattctgaaagacatgatcatccgctccaagaccctgtcgggttttgacgcgccgtatgtgccgggctgggattgccatggtttgccgattgaacacaaggtcgaagtgacccacggtaaaaacctgagcgcggataaaacccgcgagctgtgccgtgcctacgccaccgagcagatcgaggggcagaagtccgagttcatccgtctgggtgtgctgggtgatttcgccaacccgtacaagaccatggacttcaaaaacgaagccggtgaaatccgtgctttggctgagatcgtcaagggcggttttgtgttcaagggcctcaagccggtgaactggtgcttcgattgcggttcggccctggctgaagctgaagttgaataccaggacaagaagtctgcggccatcgacgttgccttcccggttgccgacgaggccaagctggccgaggcctttggtctggcggcactgagcaaacctgcttcgatcgtgatctggaccaccaccccgtggaccattccggccaaccaggcgcttaacgtacacccggaattcacctacgcgctggtcgacgtgggcgacaagttgctggtactggctgaagaactggtcgaatcgagtctggcgcgttacaacctgcagggttcggtcatcgccaccaccactggctcagcgcttgaactaatcaacttccgtcacccgttctatgaccgtctgtcgcctgtttatctggccgactacgttgagctgggtgctggcactggtgtggttcactcggctccagcctacggcgtagacgacttcgtgacctgcaaagcctatggcatggtcaacgacgacatcatcaacccggtgcaaagcaatggcgtttacgtgccgtcgctggagttcttcggtggccagttcatctggaaggccaaccagaacatcatcgacaagctgatcgaagtcggttcgctgatgttcaccgagaccatcagccacagctatatgcactgctggcgccacaagacgccgctgatctaccgtgccaccgcccagtggtttatcggtatggacaagcagccgactgatggcgataccttgcgcacccgtgcgctgcaagcgatcgaagacacccagttcgttccggcctggggtcaggcgcgcctgcactcgatgatcgccaaccgcccggactggtgcatctcgcgtcaacgcaactggggcgtgccgatcccgtttttcctgaacaaggaaagcggcgagctgcacccgcgcaccgtcgaaatgatggaagaagtggccaagcgcgttgaagtcgaaggcatcgaggcgtggttcaagctggatgctgccgagctgctgggcgacgaagcgccgctgtacgacaagatcagcgataccctcgacgtctggttcgattcgggcaccacgcactggcatgtccttcgcggttcgcacccgatgggtcatgaaaccggcccacgcgctgatctctaccttgaaggctccgaccagcaccgtggctggttccactcgtcgttgctgaccggttgcgccatcgacaaccacgcgccgtaccgcgagctgctgacccacggttttaccgtggacgaagcgggccgcaagatgtccaagtcgctgggcaacgtgattgcaccgcaaaaggtcaacgacaccctgggcgccgacatcatgcgtctgtgggttgcttcgaccgactactcgggcgaaatcgcggtttccgaccagatcctgcagcgcagtgcggacgcctaccgacgtatccgcaataccgcacgcttcctgctgtcgaacctgaccggtttcaatccagccaccgacatcctgcctgccgaagaaatgctggcactggaccgctgggcggtggatcgtgcgttgctgctgcaacgtgagctggagctgcattacggcgaataccgtttctggaacgtgtactccaaggtgcacaacttctgcgttcaggagctgggcggtttctatctcgacatcatcaaggaccgccagtacaccaccggcgccaacagcaaggctcgccgttcgtgccagaccgcgctgttccacatctctgaagcgctggtgcgctggatcgctccgatcctggcgttcaccgctgatgagttgtggcagtacctgccgggcgagcgcaacgaatcggtcatgctcaacacctggtacgaaggcctgactgaactgccggaaggcaccgaactggatcgcgcctactgggagcgaatcatggcggtcaaggttgcggtcaacaaggaaatggaaaacttgcgcgcagccaaggccattggcggtaacttgcaagcagaagtgaccttgttcgccgaagatcagctggctgctgatttgtccaagttgagcaacgaactgcgtttcgtgttgatcacctccactgccagcgttgcgccttttgcgcaggctccagcagatgccgtggttaccgaagtggctggcctcaaactcaaggtggtcaagtcggcccatgccaagtgcgcccgttgctggcactgccgtgaagacgtcggcgttaaccccgagcaccctgaaatctgcggtcgttgtgtagacaatatcagcggcgctggtgaggtacgtcactatgcctaa

SEQ ID NO: 70 - DNA, 1785 bp, unidentified
  source 1..1785: mol_type = genomic DNA; note = DP53 NADH-quinone oxidoreductase subunit C/D sequence
atgactgcaggctccgctctgtacatcccgccttacaaggctgacgaccaagatgtggttgtcgaactcaatacccgttttggccctgaggcgttcaccgcccaggccacgcgcaccggcatgccggtgctttgggttagccgcgcaaaactggtcgaagtactgaccttcctgcgcaacctgccaaaaccctacgtcatgctctatgacctgcacggtgtggacgaacgtctgcgtaccaagcgtcagggcctgccatcgggtgcagacttcaccgtcttctaccacctgatgtcgctggaacgtaacagcgacgtcatgatcaaggtggccctgtctgaaaaagacctgagtgtccctaccgtgaccggtatctggccgaacgccaactggtacgagcgtgaagtctgggacatgttcggcatcgatttcaaaggccacccgcacctgtcgcgcatcatgatgccgccgacctgggaaggtcacccgctgcgcaaggacttcccggcccgtgccacagagttcgatccgtacagcctgaccctggccaaggtgcagctggaagaggaagccgcgcgcttccgcccggaagactggggcatgaaacgctccggtgaaaacgaggactacatgttcctcaacctgggccctaaccacccttcggctcacggtgccttccgcatcatcctgcagctggacggtgaagagatcgtcgactgcgtgcctgacgtcggttaccaccaccgtggcgccgagaaaatggccgaacgccagtcctggcacagtttcatcccgtacaccgaccggatcgattacctcggcggagtgatgaacaacctgccgtacgtgctctcggtcgagaagctggccggtatcaaagtgccggatcgggtcgacaccatccgcatcatgatggccgaattcttccgtatcaccagccacctgctgttcctgggtacctatatccaggacgtgggcgccatgaccccggtgttcttcacgttcaccgaccgtcagcgcgcttacaaggtgatcgaggccatcaccggtttccgtctgcacccggcctggtaccgcatcggcggcgttgcccacgacctgccgaacggctgggatcgcctggtcaaggaattcatcgactggatgcccaagcgtctggacgagtaccagaaagccgctctggacaacagcatcctgcgtggtcgtaccatcggcgttgccgcctacaacaccaaagaggccctggaatggggcgtcaccggtgccggcctgcgctccaccggttgtgacttcgatatccgcaaggcgcgcccgtattccggctacgagaacttcgaattcgaagtcccgctggcagccaacggcgatgcctacgatcgttgcatcgtgcgcgtcgaagaaatgcgccagagcctgaaaatcatcgagcagtgcatgcgcaacatgccggccggcccgtacaaggcggatcacccgctgaccacgccgccgcctaaagaacgcacgctgcagcatatcgagaccttgatcacgcacttcctgcaagtttcgtggggcccggtgatgccggccaacgaatccttccagatgatcgaagcgaccaagggcatcaacagttattacctgacgagcgatggcggcaccatgagctaccgcacccggattcgcaccccaagcttcccgcacctgcaacagatcccttcggtgatcaaaggtgaaatggtcgcggacttgattgcgtacctgggtagtatcgatttcgttatggccgacgtggaccgctaa

SEQ ID NO: 71 - DNA, 1119 bp, unidentified
  source 1..1119: mol_type = genomic DNA; note = DP53 Protein RecA sequence
atggacgacaacaagaagaaagccttggctgcggccctgggtcagatcgaacgtcaattcggcaagggtgccgtgatgctgatgggcgaccaggagcgtcaggcagtcccggcgatctccaccggctccctgggtctggacatcgcactgggcattggcggtctgccaaaaggccgtattgttgaaatctacggccctgagtcgtcgggtaaaaccacactgaccctgtccgtgattgcccaggcgcaaaaggccggtgctacctgcgccttcgtcgatgccgagcacgcccttgatcctgagtacgctgccaaactgggcgtaaacgttgatgacctgctggtttcacagcctgacaccggcgaacaggcactggaaatcaccgatatgctggtgcgttccaatgcggttgacgtgatcatcatcgactccgttgctgcactgacgccaaaagctgaaatcgaaggcgacatgggcgatacccacgttggcctgcaagcccgtctgatgtcgcaagcgctgcgtaaaatcaccggtaacatcaagaacgccaactgcctggttatcttcatcaaccagatccgcatgaaaatcggcgtgatgttcggcagccctgaaaccaccaccggtggtaacgcactgaagttctacgcttcggtacgtctggatatccgccgcaccggcgccgtaaaagaaggcgatgtggtggtgggtagcgaaacccgcgtgaaagtggtcaagaacaaggtggcaccaccgttccgtcaggctgaattccagatcctgtacggcaagggtatctacctgaacggtgaaatgattgacctgggcgtactgcatggctttgttgaaaaagctggcgcctggtacagctacaacggcagcaaaatcggtcagggcaaggccaactccgccaagttcctggacgataacccggacatcaaggatgcgctggagaagcagctgcgtgagaagttgctcgggccaaaaaccgatgccgaactggcagcgacggactgcaatggacctgctcgcgcgacgcgagcacggtcgagtcgagctgacgcgcaagttgcgtcagcgcggcgcttgccccgacatgatcgacgctgcccttga

SEQ ID NO: 72 - DNA, 1848 bp, unidentified
  source 1..1848: mol_type = genomic DNA; note = DP53 RNA polymerase sigma factor RpoD sequence
atgtccggaaaagcgcaacagcagtctcgtatcaaagagttgatcaccctcggccgtgagcagaagtatctgacttacgcagaggtcaacgaccacctgcccgaagatatttcagatccggagcaagtggaagacatcatccgcatgattaatgacatggggatccccgtacacgagagtgctccggatgcggacgcccttatgttggccgatgccgacaccgacgaagcagcagctgaagaagcggctgcagcgttggcggcagtagagaccgacattggtcgtactaccgaccctgtgcgcatgtatatgcgtgaaatgggcacggtagaactgctgacacgtgaaggcgaaatcgaaatcgccaagcgtatcgaagaaggcatccgtgaagtgatgggcgcaatcgcgcacttccctggcacggttgaccatattctctccgagtacactcgcgtcaccaccgaaggtggccgcctgtccgacgttctgagcggttatatcgacccggacgacggtattgcgccgcccgcagccgaagtacctcctcctgtcgacaccaaggtgaaagccgaaggtgatgacgaagaggacgacaaggaagattccggcgaagacgaggaagaggtcgaaagcggccctgatccgatcatcgcggcccagcgctttggcgctgttttcgatcagatggaaatcgctcgcaaggccctgaaaaagcacggtcgcggcagcaagcaggcaattgccgagctggttgcactggctgagctgttcatgccgatcaaactggttccgaagcaattcgaaggcctggttgagcgtgttcgcagcgccctggagcgtctgcgtgcacaagagcgcgcaatcatgcagctgtgtgtacgtgatgcacgcatgccgcgcaccgatttcctgcgtctgttcccgggcaacgaagtcgacgaaagctggagcgatgcgctggccaaaggcaaaagcaaatatgctgaagccattggtcgcctgcaaccggacatcatccgttgccagcaaaagctctctgctctggaagcagaaaccggcttgaagattgccgagatcaaggacatcaaccgtcgcatgtcgatcggcgaggccaaggcccgccgcgcgaagaaagaaatggttgaagccaacttgcgtctggtgatctccatcgccaagaagtacaccaaccgtggcctgcagttcctcgatctgatccaggaaggcaacatcggcttgatgaaagcggtagacaagtttgaataccgccgcggctacaaattctcgacttatgccacctggtggatccgtcaggcgatcactcgctcgatcgccgaccaggcccgcaccatccgtattccggtgcacatgatcgagacgatcaacaagctcaaccgtatttcccgtcagatgttgcaggaaatgggccgtgaaccgaccccggaagagctgggcgaacgcatggaaatgcctgaggataaaatccgcaaggtattgaagatcgctaaagagccgatctccatggaaaccccgatcggtgatgacgaagactcccatctgggtgacttcatcgaagactcgaccatgcagtcgccaatcgatgttgctaccgttgagagccttaaagaagcgacacgcgacgtactcggcggcctcacagcccgtgaagccaaggtactgcgcatgcgtttcggtatcgacatgaataccgaccacacccttgaggaggttggtaaacagttcgacgttacccgtgagcggattcgtcagatcgaagccaaggcgctgcgcaagctgcgccacccgacgagaagcgagcatttgcgctccttcctcgacgagtga

SEQ ID NO: 73 - DNA, 4073 bp, unidentified
  source 1..4073: mol_type = genomic DNA; note = DP53 DNA-directed RNA polymerase subunit beta sequence
atggcttactcatatactgagaaaaaacgtatccgcaaggactttagcaagttgccggacgtcatggatgtgccgtatctcttggcaatccagctggattcgtatcgtgaattcttgcaggcgggagcgactaaagatcagttccgcgacgtgggcctgcatgcggccttcaaatccgttttcccgatcatcagctactccggcaatgctgcgctggagtacgtcggttatcgcttgggcgaaccggcatttgatgtcaaagaatgcgtgttgcgtggcgtaacgtacgccgtacctttgcgggtaaaagttcgtttgatcattttcgacaaagaatcgtcgaacaaagcgatcaaggacatcaaagagcaagaagtctacatgggtgaaatccccctgatgactgaaaacggtaccttcgtaatcaacggtaccgagcgtgtaattgtttcccagctgcaccgttccccgggcgtgttctttgccacgaccgcggcaagacgcacagctccggtaagctgctttattccgcgcgtatcattccttaccgtggttcgtggctcgacttcgagttcgacccgaaagactgcgtgttcgtgcgtattgaccgtcgtcgcaagctgcctgcatcggtattgctgcgcgcgctgggttataccactgagcaagtgctggacgcgttctacaccaccaacgtgttccacgttcagggtgagagcatcagcctggagctggttccacagcgtctgcgcggtgaaatcgcggccatcgacattaccgatgacaaaggcaaggtgattgttgagcagggtcgtcgtatcactgctcgtcatatcaaccagctggaaaaagccggtgtcaaagagctcgttatgcctctggactatgtcctgggtcgcacaacggccaaggctatcgtgcatccggctactggcgaaatcattgctgagtgcaacaccgagctgaccactgaaatcctggcaaaagttgccaagggccaggttgttcgcatcgaaacgttgtacaccaacgatatcgactgcggtccgttcgtctccgacacgctgaagatcgactccaccagcaaccaactggaagcgctggtcgaaatctatcgcatgatgcgtccaggcgagccgccaaccaaagacgctgccgagactctgttcaacaacctgttcttcagccctgagcgctatgacctgtctgcggtcggccggatgaagttcaaccgtcgtatcggtcgtaccgagatcgaaggttcgggcgtgttgtgcaaagaagacatcgttgccgtgctgaagaccctggtcgacatccgtaacggtaaaggcatcgtcgatgacatcgaccacctgggtaaccgtcgtgttcgctgtgtaggcgaaatggccgagaaccagttccgcgttggcctggtacgtgttgagcgtgcggtcaaagagcgtctgtcgatggctgaaagcgaaggcctgatgccgcaagacctgatcaacgccaagcctgtggctgcggcggtgaaagagttcttcggttccagccagctgtcccagttcatggaccagaacaaccctctgtccgagatcacccacaagcgccgtgtttctgcactgggcccgggcggtctgacgcgtgagcgtgcgggctttgaagttcgtgacgtacacccgactcactacggccgtgtttgccctattgagacgccggaaggtccgaacatcggtctgatcaactccctggctgcctatgcgcgcaccaaccagtacggcttcctcgagagcccgtaccgtgtagtgaaagacgcactggtaactgacgagatcgttttcctgtccgccatcgaagaagctgatcacgtgatcgctcaggcctcggccacgatgaacgacaagaaagtgctgatcgacgagctggttgctgttcgtcacttgaacgaattcaccgtcaaggcgccggaagacgtcaccttgatggacgtttcgccgaagcaggttgtttcggttgcagcgtcgctgatcccgttcctggaacacgatgacgccaaccgtgcgttgatgggttccaacatgcagcgtcaagctgtaccaaccctgcgcgctgacaagccgctggtaggtaccggcatggagcgtaacgtagctcgtgactccggcgtttgcgtcgtggctcgtcgtggcggcgtgatcgactctgttgatgccagccgtatcgtggttcgtgttgctgatgacgaagttgaaactggcgaagccggtgtcgacatctacaacctgaccaaatacacccgttccaaccagaacacttgcatcaaccagcgtccgctggtgcgcaagggtgaccgtgtacagcgtagcgacatcatggctgacggcccgtccaccgatatgggtgaactggcgctgggtcaaaacatgcgcatcgcgttcatggcctggaacggttacaacttcgaagactccatctgcttgtcggaacgagttgttcaagaagaccgctttaccacgatccacattcaggaactgacctgtgtggcacgtgacaccaagcttgggcctgaagagatcactgcagacatccctaacgtgggtgaagctgcactgaacaaactggacgaagccggtatcgtttacgtaggtgctgaagttggcgccggcgacattctggtaggtaaggtcactccgaaaggcgagacccagctgactccggaagagaagctgttgcgtgccatcttcggtgaaaaagccagcgacgttaaagacacctccctgcgcgtacctaccggtaccaaaggtactgttatcgacgtgcaggtcttcacccgtgacggcgttgagcgtgatgctcgtgcactgtcgatcgagaagacccagctggacgagatccgcaaggatctgaacgaagagttccgtatcgttgaaggcgctaccttcgaacgtctgcgctctgctctggttggccgcattgccgaaggtggtgccggtctgaagaaaggtcaggaaatcaccaatgaaatcctggacggtcttgagcatggtcagtggttcaaactgcgcatggctgaagatgctctgaacgagcagcttgaaaaggctcaggcttacatcatcgatcgccgtcgtctgctggacgacaagttcgaagacaagaagcgcaaactgcagcagggcgatgacctggctccaggcgtgctgaaaatcgtcaaggtttacctggcaatccgccgtcgcatccagccgggtgacaagatggccggtcgtcacggtaacaagggtgtggtctccgtgatcatgccggttgaagacatgccgtacgatgccaatggcaccccggttgatgtggtcctcaacccgttgggcgtaccttcgcgtatgaacgttggtcagattctcgaaactcacctgggcctcgcggccaaaggtctgggcgagaagatcaacctcatgattgaagaacaacgcaaggtcgctgacctgcgtaagttcctgcatgagatctacaacgaaattggcggtcgtcaagaaagcctggatgacttctccgatcaggaaatcctggatctggcgaagaaccttcgcggcggtgtgccaatggctaccccggtgttcgacggtgccaaggaaagcgaaatcaaggcaatgcttcgtttggcagacctgccagacagcggccagatggtgctgactgatggtcgtaccggcaacaagttcgagcgtccggttaccgttggctacatgtacatgctgaagctgaaccacttggtagacgacaagatgcacgctcgttctaccggttcttacagcctggttacccagcagccgctgggtggtaaggcgcagttcggtggtcagcgtttcggggagatggaggtctgggcgctggaagcctacggcgcggcatacactctgcaagaaatgctcacagtgaagtcggacgatgtgaacggccgtaccaagatgtacaaaaacatcgtggacggcgatcaccgtatggagccgggcatgcccgagtccttcaacgtgttgatcaaagaaattcgttccctcggcatcgatatcgatctggaaaccgaataa

SEQ ID NO: 74 - DNA, 2073 bp, unidentified
  source 1..2073: mol_type = genomic DNA; note = DP9 Glycine--tRNA ligase beta subunit sequence
atggcacataattatttactagaaattggattggaagaaattccggcccatgttgtaactccaagtatcaaacagttagtacaaaaagtaacagccttcttaaaagaaaatcgcttaacatacgactcaattgatcatttttcaactcctcgtcgtttggcaattcgaatcaatgggttaggcgaccaacaacctgatattgaagaagatgctaaaggccctgctcgtaaaattgctcaagatgctgatggaaattggactaaggctgcaattggctttacacgtggacaaggtcttacggttgacgatattacttttaaaacaatcaaaggtacggactatgtgtacgtccataagttaatcaaaggaaagatgactaaggaaatccttacggggataaaagaagttgttgaatcaattaatttcccaacaatgatgaagtgggctaactttgattttaaatatgtacgcccaattcgttggctggtttctattctagatgaagaagtccttccttttagtatcttagacgtaactgcgggacgccgaacagaaggacatcgtttcttaggtgaagctgtcgaactggctaatgctgaagaatatgaagcaaaattacacgatcaatttgtgattgttgatgccgacgagcgtaaacaattaatttcaaaccaaattaaagcaattgctgaaagcaatcgttggaacgttacccctaacccaggtcttttagaagaggttaacaatttggttgagtggccaaccgcttttaatgggggatttgatgaaaagtatttagctattccagaagaggtattgataacatcaatgcgtgaccaccaacgcttcttctttgtccgcgaccaagctggaaagctattgccaaacttcatctccgtacgaaatgggaatgaagaatttattgaaaatgttgttcgtggaaatgaaaaagttttaactgcacgtttagaagacgctgctttcttctacgaagaagatcaaaaacatgatattaattattatgttgaccgacttaaaaaggttagtttccatgataagattggttcaatgtacgaaaaaatgcaacgagttaattctattgctaaagttattggaaacaccttaaatcttaatcaaacggaacttgatgatatcgatcgcgctacaatgatttataaatttgatttggtaactggtatggttggtgagttctcagaattacaaggagtaatgggtgaaaaatatgctcaacttaatggtgaaaaccaagcagtagcccaagccattcgcgaacattacatgccaaatagcgcagaaggtgatttgcctgaaagtgtaacgggcgcggtagtcgcattagctgataagtttgataacatctttagttttttctcagctggtatgattccaagtggttcaaacgatccatatgcattacgccgacatgcatatggaattgttagaatcttaaatagccgtgattggcaattagatttaaatcaattcaaatcacaatttaagactgaattagcggagaatggcacagcgtttggtgtggatgtcgatcaaaactttgaccaagtacttaacttctttaatgaccgtattaaacaattgcttgatcatcaaaagattagtcatgatatcgttgaaacggtgcttacaggtaataatcatgatgttacggaaattatcgaagctgcccaagtactagcagatgctaaagcgagctctacatttaaagatgatattgaagctttaacacgagttcaaagaattgctacaaagaatgaagaaagtggagaacttaatgtagatccacaattatttaataatgcttctgaaggcgaactttttgatcaaattattaaaattgaagctgcaaataatttgacaatgagccaactatttgctaaattatgcgagttgactcctgcgattagcaagtactttgacgcaacgatggtcatggacaaagacgaaaatattaagtgtaatcgtttgaatatgatgagtcggttagctaatttaattctaaaaattggggatctaactaacgtacttgtaaaataa

SEQ ID NO: 75 - DNA, 1341 bp, unidentified
  source 1..1341: mol_type = genomic DNA; note = DP9 Glutamine synthetase sequence
atggcaaagaaaaattattcgcaagcagatattcgtcagatggcaaaggatgaaaatgtacgttttctccgattaatgtttacagatctttttggaataattaagaacgttgaagtaccaattagtcaattggacaaactattagataataaattgatgtttgatggttcctcaattgacgggtttgttcggattgaagaaagtgacatgtatttatacccagatctttctacttggatggttttcccatggggaagcgaacatggcaaggtggctcgcattatttgtgaagtatactcaaatgatcgtaaaccattcgtgggtgatccacgtaacaatttaattcgagtactccaagagatgaaggatgcaggatttactgattttaatatcggacctgaacctgagtttttcttgttgaaattagatgaaaatggtaaaccaaccactaatttaaatgataaaggtagttactttgatttagctcctgttgatttaggtgaaaactgccgtcgtgatattgttttggaacttgaaaatatgggctttgatgttgaagcttctcatcatgaagttgctccaggacaacacgaaattgactttaaatacgccgatgctttgaccgctgccgataacattcaaacctttaagttggttgttaagacagttgcccgtaaatataacctgcatgctacatttatgcctaaacctatggatggaatcaatggttcagggatgcatttaaacatgtcacttttcaataaggaaggcaatgctttctatgacgaaaagggtgacttacaactttctcaaaatgcttactggttccttggtggactattgaagcatgctcgtagttatacggccgtatgtaacccaattgttaactcgtacaaacgtttagttcctggatatgaagctccagtatacgttgcttggtcaggttcaaatcgttcaccacttattcgcgttccttcaagtaagggactctcaactcgttttgaagttcgaagcgtcgatccagctgctaacccatacttagcaattgcatcagtattggaagcaggcttagatggcattagaaacaagattgaaccagaagattccgttgatcgtaatatctatcgaatgaacattcaagaacgtaatgaagagcatattacagatctaccttcaacattacacaatgctttgaaggaattccaaaatgatgatgtaatgcgtaaggcattaggagatcacattttccaaagcttcctcgaagctaagaagttagaatgggcttcttaccgtcaagaagtgacacaatgggaacgtgatcaatatctcgaaatgttctag

SEQ ID NO: 76 - DNA, 1947 bp, unidentified
  source 1..1947: mol_type = genomic DNA; note = DP9 DNA gyrase subunit B sequence
ttggcagacgaaaaagaaacgaaagcagaattagccagagaatatgatgcgagtcaaattcaggttttagaggggctcgaagcagttcgtaaacgcccaggaatgtatattgggtcgactagttctcaaggactacaccatttggtttgggaaattattgataatggtattgatgaagctcttgcaggatttgcagacaaaattgatgtgatcgttgaaaaagacaatagtattaccgtcactgataatggacgtgggattccggttgatatccaaaagaaaactggaaaaccagctttagaaacagtctttacggtcctacatgccggaggtaaattcggcggtggcggttataaagtttctggaggattgcatggtgtgggcgcatccgttgtaaatgcgttatcaacggaattagatgcgcgcgtcatgaaggacggtaaaatctattacattgattttgcgctaggaaaagtaaaaacaccgatgaaaacgattggtgatactgaacatcctgacgatcatggaactattgttcatttcgttccagatccagatattttccaagaaactaccacatacgacattaatatcttaaaaacacgaattcgtgaattagcctttttgaacaaaggtctacggattactttgaaggatatgcgtcctgaaaagccaactgaagacgacttcttgtatgaaggtgggattcgccactacgttgaatatctaaacgaaggcaaagaagtaattttccctgaacctatctatgttgaaggggttacaaaaggtatcactgttgaagtagctatgcaatatatcgaaggttatcaaagtaaattgttaacttttactaacaatattcatacttacgaaggcggtacccacgaagaaggtttcaaacgtgctttaacacgagttattaacgattacgctaaaaacaacaatattttaaaagaaaatgatgataaattgtctggtgatgatgttcgagaaggtttgacggcagtagtcagcgttaagcatcctgatcctcaattcgaaggacaaacgaaaacaaaattgggtaactcagatgctcggacagctgttaacgaagtgtttgctgaaactttcaataaattcttattggaaaatcctaaggttgcacgtcaaattgttgataagggaatcttggcagcaaaagcaagagtcgccgctaaacgagctcgtgaagttacgcgtaagaagagtggcctagaactcaataatcttcctggtaaattagctgataatacttctaaggatccttcaattagtgaattattcattgtcgagggtgattctgccggtggtagtgctaagtcgggacgttcgcgtctcacacaagctattttgccaattcgtgggaagattttgaacgttgaaaaagccactttggatcgggttttggccaatgaagaaattcgttcactctttacagcgctcggaactggatttggtgaggactttgatgtaagtaaagccaactatcataaattgattatcatgaccgatgccgatgtcgatggtgctcatattcggacactattattgacgctgttctatcgttacatgcgtccaatgattgatgcaggatttgtttacattgctcaaccaccgctctaccaagtacgtcaaggtaagatgattcaatatatcgattctgatgaagaattagaaacagtacttggacaattgtcaccatcaccaaaacctgtaattcaacgttataaaggtcttggtgaaatggatgctgagcaactttgggaaacaaccatgaatccagaaaatcgacgcttgttacgagtttcagccgaagatgctgatgctgcaagtggtgattttgaaatgttgatgggtgacaaggttgaaccacgtcgtaaattcattgaagagaacgctgtgtttgttaaaaacttggatatctaa

SEQ ID NO: 77 - DNA, 2418 bp, unidentified
  source 1..2418: mol_type = genomic DNA; note = DP9 Leucine--tRNA ligase sequence
atggcttataatcataaagatatcgaacagaagtggcagcaattctggagcgacaatgagacttttaagacggtcgaagatgcagacaaacccaaatattatgcattagacatgttcccttatccatcaggtcaaggactccatgtgggccatcctgaaggatatacagcaacagatattatgtcacgaatgaaacggatgcaaggttacaaagtacttcatccaatgggatgggatgcttttggtcttccagcagaacaatatgcgatgaagacgggtaacaatccgcgtgattttacagctaagaatattcaaaactttaagcgtcaaatccaatcacttggtttttcttatgactggtcgcgagaagttaatacaactgatccagcttactacaagtggactcaatggatttttgagcaactctacaagaagggcttagcttatgaaaaagaaacgctggtaaactgggctcctgatttaatgggtggaacggtagttgctaacgaagaagttgtggatggtaagacagaacgtggtgggttccccgtttatcgtaaaccaatgaaacaatggattcttaaaattacagcttacgccgaccgtttgattgacgatttggacctggtagattggcccgatagtattaaagaaatgcaaaaaaactggattggtcgttcagtgggggctagcgtcttctttaatgttgaagatagcgaaaaacaaattgaagtatttacaacgcgtccagatacattatttggcgcaacatacttggtaatttcaccagaacatgacctcgttgaccaaattacaactccagaaagtaaagctgccgttgaagaatacaagaaagctgttgcaactaaatcagatcttgaacggacggatttgagtaaagataagacgggagtctttacgggagcatacgcggttaaccctgttaatggtaagaaaattccagtttggattagtgattacgtattggcttcatacggaactggagcagtgatggctgttcctgctcatgatggccgtgactacgaatttgctaagaaattcaagatagatatggtgccagtttatgaaggtggcaatcttgaagatggagtattggacagcgaaggcgggctaattaactctggattcctagatgggatggataagcagacggctattgataccatgattagctggttggaagaacatggagttggtcataagaaggttaactatcgtcttcgtgactgggtcttctctcgccaacgctactggggtgaaccaatccctgtaattcattgggaagatggagaaacaactttgattcctgaagatgaattgccattgagactcccggctgcaactgacattcgtccttccggtaccggagaaagcccattagctaacctagatgattgggtaaacgtagttgatgaaaatggtcgtaagggtcgccgggaaactaatacaatgccacaatgggcgggtagttcatggtacttcctccgttacgttgatcctaagaatgatcaaaagattgctgacgaagatttacttaaagaatggttaccagtcgacttatatgttggtggagctgaacatgcggtacttcatttactttatgcacgtttctggcacaaagttttatatgatctaggagttgtaccaactaaggaaccattccaaaaattggtcaaccaagggatgattctcggtagcaatcatgagaagatgtctaagtcaaaagggaacgtggttaatccagatgatattgttgagcgctttggagcggatactttacgattatacgaaatgttcatgggacctctgacagaatcagtcgcctggagtgaagatgggcttaacggaagtcgtaagtggattgaccgcgtctggcgcttgatgattgacgacgaaaaccaattgcgtgatcatattgttactgaaaatgatggcagtttggatatgatttataaccaaactgttaagaaggtaactgatgattatgaaaacatgcgctttaacacggctatttcacaaatgatggtctttgttaatgaagcatacaaggctgataaacttccagcagtatatatggaaggattagttaagatgttagctccaattattccgcacgttgctgaagaactttggagtttgctaggtcacgaaggtggtatttcatacgctgaatggccaacatatgatgaaagtaagttagtagaagctacagttcaagtcattctacaagttaatggtaaagttcggagtaaaattaccgttgacaaggatatcgccaaagaagaacttgaaaaattagcgttagctgatgctaagattcaacaatggacggcagataagactgttcgtaaggtaattgttattcctaacaagattgttaatatcgtagtaggctaa

SEQ ID NO: 78 - DNA, 1350 bp, unidentified
  source 1..1350: mol_type = genomic DNA; note = DP9 Glucose-6-phosphate isomerase sequence
atggcacatatttcatttgacagttctaatgttgcagattttgtacatgaaaacgaacttgcagaaatccaaccacttgttacagctgctgatcagattttacgtgatggctctggcgctggtagtgatttccgtggatggatcgatttaccatcaaattatgataaggacgaatttgcccgtatcaagaaagccgctgataagatccgcaatgactcagaagtattcgttgctatcggtattggtggttcatatttgggtgctcgtgcagccattgatttcttgaacaacactttctacaatcttcttactaaagaacaacgtaatggtgctcctcaagtaatcttcgctggtaactcaattagttcaacttaccttgctgacgtattgaacttaatcggggaccgtgacttctcaattaacgtaatttctaagtcaggtacaactacagaaccagctattgcattccgtgttcttaaagaaaaactaatcaagaagtacggtgaagaagaagctaagaaacgtatctatgcaacaactgaccgtgctaaaggcgccctaaagacagaagctgatgcagaaaactatgaagaattcgtagttcctgatgacattggtggtcgtttctctgttctttcagctgttggtttattaccaatcgcggttgccggtggcgatattgaccaattgatgaagggtgctgaagatgcaagcaacgaatacaaggatgctgatgttacaaagaacgaagcatacaagtacgctgctttacgtaacatcctttatcgtaagggctacacaacagaacttcttgaaaactacgaaccaacacttcaatacttcggcgaatggtggaagcaattgatgggtgaatcagaaggtaaagatcaaaagggtatctacccatcttctgctaacttctcaactgacttacattcactaggacaatacatccaagaaggtcgtcgcaatttaatggaaacagttatcaatgttgaaaagcctaaccatgacatcgacattcctaaggctgaccaagaccttgatggattacgttatctcgaaggtcgcacaatggacgaagttaacaagaaagcttaccaaggtgtaactcttgctcataacgacggtggtgttccagttatgacggttaacattcctgatcaaacagcttacacattaggctatatgatttacttcttcgaagcagctgttgctgtatctggttacttgaacggaattaatccattcaaccaaccaggtgttgaagcatacaagtcaaatatgtttgcattacttggtaaaccaggttatgaagataagacagctgaattaaacgctcgtctataa

SEQ ID NO: 79 - DNA, 1725 bp, unidentified
  source 1..1725: mol_type = genomic DNA; note = DP9 Phosphoglucomutase sequence
atgagttgggaagattctgtcaaagaatggcaagattatgcagatttagattttaatttaaaaaaagaattagcaactttagctgaagataaagatgctttaaaagaagccttttatgctccaatggaatttggtacagcaggaatgcgtggcgtaatgggccctggtatcaaccggatgaatatctatacggttcgtcaagcaacagaaggtttagctaattttatggataccttagattttactgataagaaacggggagtggcgatcagttttgattcccgctatcactcacaagagtttgctttagcagcagctggtgttttaggtaagcatggtattccaagttttgtttttgatagtatgcgtcccactccagaattatcatatacagtacgtgagttaaacacttatgctggaatcatgattactgctagtcataatcctaaacaatataatggatataagatttatggtcctgatggcggacaaatgccaccaatggaatctgataagattacagaatatattcgccaagtaactgacatctttggtgttgaagctcttactcaaagtgaattaagagctaagggcttaatgaccattattggtgaagacattgacctcaagtatcttgaggaagttaagacggtatcaattaatcatgaactaatccagcgctttggtgcagacatgaagttgatctactcaccattacatggtactggaaaagtagttggtggacgtgcgttagaaaatgctggttttaaggattacactatggtccctgaacaagcaattgctgacccagaatttattacaacgccattccctaacccagaattcccacaaacttttgatttggctattgaattaggtaaaaagcaagatgctgaccttttgattgccactgatccggatgccgatcgtttgggagctgccgttcgtttaccaaatggtgactacaaattattgacagggaaccaaattgcagccttgatgttagaatacatcttaactgcgcatgatgcagcaggtgacttgccaggtaacgcagctgccgttaagtcaattgtttctagtgaactagcaaccagaattgccgaagcccatcatgtagaaatgattaacgttctaactgggtttaagtacattgctgaccaaattaaacattacgaagaaaatggcgaccatacctttatgtttggtttcgaagaaagttatggctatcttgttcggccatttgttcgcgataaagatgccatccaaggaattgtcctattggctgaaattgctgcttattatcgtagtaaggggcaaaccttatatgacggtcttcaaaacttatttactacttacggatatcatgaagaaaagaccatttcaaaagatttccctggagttgacggtaaagaaaaaatggctgccattatggaaaaggttcgtgaagaacgcccaagtcaatttgatcagtacaaggtattagaaactgaagacttcttagctcaaactaagtatgaagcagatggatctacccaagctatcaaattaccaaaagcggatgttttgaaatttacattagatgatggtacttggattgcaattcgtccttctggaacagaaccaaaaattaaattctatattggtacagttggcgaagatgaaaaagatgctttgaataagattgatgtttttgaaacagctattaatgaacttataaaataa

SEQ ID NO: 80 - DNA, 3426 bp, unidentified
  source 1..3426: mol_type = genomic DNA; note = DP9 2-oxoglutarate carboxylase small subunit sequence
atgcaccgtattttaattgccaaccgaggcgaaattgcgacccgaattattcgggcaacgcatgaactcggaaaaacagctgtagcaatttatgctaaagcggatgaattttctatgcatcgttttaaagcagatgaagcttaccaagttggtgaagatagtgatccaattggagcatatttaaatattgatgacattattcgtattgcaaaagaaaataatattgatgcaattcaccccggctatggatttttgtcggaaaatgctgtatttgcgcgagcagttgaagcagctgggattaagttcattggacctcgacccgaattactagaaatgtttggtgataaattacaagctaaaaatgcagccattaaggccggtgtaccaactattccgggaacggaaaaaccagttaaagatgtcgatgacgcgctaaattttgcagagcaatttggctatcctatatttgttaagtcagcggcaggtggcggcggaaaagggatgcggattgtacatcatcaacaagagatgcgcgaagcatttaagatggctcagtcagaagcttcttcgtcttttggtgacgatgaaatttacttagaacgttacttagttgatccaatccatattgaggttcaagtagttgcggatgaacacggtgagatggttcatttgtatgaacgaaattcatcgattcagcgacgccatcaaaaaatcattgaatttgctccagcagtgggaatttctgccaccgtccgtgatcaaataagaaaagctgctttaaaattattgaagtcggtcaattatagtaacgctgcaaccattgagtttttggtagaaggtaatcaattttactttatggaagtgaatccacgaattcaggttgaacatacagttaccgaagaagtcacgggaatcgatattgtgcaaacccaaattaaggttgctgaaggtcaaagattacacgaagaaatcggtgttcctcaacaagcccaaattgaagctgtgggagtggcaattcaagcccgaattaccactgaagatccaatgaataactttattccagatgtcggtagaatccagacgtatcgttcacctggtggaacaggtgtgagattggatgctggaaatgcctttgctggagccattgtaactccgcattatgattcacttctgaccaaggcaattgtccatgcgccaacctttgacgaagccttggtaaagatggatcgagtgctcaatgaatttgtaattgctggggttaaaactaatattccatttttaaagaaattaattcatcatcctatttttagatcggaattagctccgacaacctttgtggatgagacaccagaactctttgatttaaaagctgaaactccggtagttactcaacttttgagttacattgctaatactactatcaatggttatccaggcttagaaaagcagaatccagtagtgttaactcggccagtccgtccacattttgaagcacaagtaccgcatgaaaatgcgaaacagatcttggatagtaagggacctgatgccatgatcaattggctgttaaaacaaaagcaggtcttgctaaccgatacgaccatgcgggatgcccatcaatcattatttgctacgcgaatgcggaccaaagacatggtagaaattgccgatcaagtccagaaaggtctgcctaacctattttcagctgaagtttggggcggtgcgacctttgatgttgcttatcggttcctaggtgaggatccatgggaaagactccaacaattgcgggctaaaatgccaaatacgatgctccaaatgcttttacgtgggtcaaatgcagtagggtatcaaaattatccagacaacgccattgacgaatttattcgattggctgccaaaaatggaattgatgttttccgaatctttgattctcttaattgggtgccacagcttgaagaatctatccaacgggtgcgtgataatggaaaagtggctgaagcagccatggcatatactggcgatattttagatactaatcgtactaaatataatttgaaatattatgtggatttggctcaagaactccaagcagcaggtgctcatattattggaatcaaagatatgtcaggaattttaaaaccacaagctgcttatgcattaatttcagagttaaaaaatcatctggatgtgccaattcatttgcatacgcacgatactacaggcaacggcattttcttatattctgaagcaatacgagctggagttgatgtggtcgacgttgccacttctgcgctagcgggaacgacttctcagccttcaatgcagtctctttactatgcgttgtctaataaccagcgccaaccagatttagatattcaaaaagcagaaaaactagatgaatattggggcggaattcgaccatattacgaaggatttggcacccaattaaatggaccacaaactgaaatttatcgaattgaaatgcctggtggacagtataccaaccttcgccagcaagctaacgcagtccatttgggtaagcgttgggatgagattaaggaaatgtacgcaaccgtcaatcaaatgtttggcgatattccaaaggttacgccttcttctaaagtagttggcgatatggcactattcatggtccaaaatgatttgacgcctgaaatggtaatgaacgataagggacaattaagttttcccgaatcagtggtaaactttttccgtggtgatttaggacaaccggcgggtggttttccaaaacagctccaaaaggtgattctaaaagagcaagccccattgacagtacgaccaggagctttagccgatccagttgattttgatcaagttcgtaaacaggcaactaaggttttaggtcaccaagcaagtgatgaagaagttatgtcgtttattatgtatccagatgtgatgaccgaatacattcaacgtcaaaatgaatatggtccagtaccattattagatactccaatctttttccaaggcatgcatattggccaacgcattgatttacaattgggacgcggaaaatcggtcattattgtccttcgagaaattagtgaagcagatgaggcgggccaaaggtcacttttctttgatataaatggacaaagtgaagaagtgattgtttatgatgttaatgcgcaggtaacgaaagtaaagaagattaaagctgatccgactaaagccgaacagattggcgctactatggcgggctcggtcattgaagtccaagtagaagcgggccaaaaggtccagcgaggtgataacttaattgtcactgaggcgatgaaaatggagaccgcgttaagagcacctttcgacgcaaccattaagaagatttatgctacccctgaaatgcaaatcgagacgggggatttattgattgaactagaaaaggagtaa

SEQ ID NO: 81 - DNA, 2058 bp, unidentified
  source 1..2058: mol_type = genomic DNA; note = DP3 Glycine--tRNA ligase beta subunit sequence
atgtcaacatttttattagaaattggacttgaagaaataccagctcatttggtaaccagttcagagaatcagttaattgaaagaactaaaaagttcttatcagagcatcgtttaacagtaggtgatattaaaccatattcaacaccgcgacgtctggctgtcgttttgacagatgttgctgaaacatcagaaagtttaagcgaagaaaagcgtggaccatctgttgaccgtgcacaagacgaaaacggtaattggacaaaggcagcattaggttttgcacgtggtcaaggtgctaatcctgaagcatttgaaattaaagatggatatgtttggctaacaaaacgtactgctggtgtagccgcgaatgaaattttagctaaaattggtgatgaagttgtcgcccaaatgaaattttcaacttatatgaagtgggctaatcacagctttttgtatgttcgacctattcgttggctcgtagcacttcttgatagtgaagtcatttctttcaacgtgttagatattaccacagatcgtttcacacgtggtcatcgttttttgtcttcagaacatgttgaaatatcttctgcagataattatgtaacgactttgcagggtgctaacgtggttgttgatgctacagtgcgcaaaaatgaaattcgatcgcagttgaatgcaattgctgaagctaatggttgggttctgcaacttgagaccgatgcggcgcaagatttgttggaagaagttaataacattgttgagtggccaacagcgtttgctggcagtttcgatgagaaatatttagaaataccagatgaagttttgattacatcaatgcgcgaacatcagcgtttcttctttgtgacgaatgaaaaaggacaattattgccacactttttgtcaataagaaatggtaaccgtgagcatctaaacaacgttattgctggaaatgaaaaagtattggtagcaaggttagaagatgccgaattcttctatcatgaagaccaaaccaaatcaatttctgattacatgactaaagttaaaaagttagtcttccatgaaaaaattggtacggtgtatgaacacatgcaacgcactggtgctttggcttcagcaatggcggtggttttgaagtttgatgaagtacaacaggctgatttgacccgtgcatcagaaatttataaatttgatttgatgaccggtatggttggtgaatttgatgaacttcaaggcattatgggtgagcattatgccaagctttttggcgaagatgatgcggttgcaacagccattcgagagcattatatgccaacttcagctaatggtgaggttgcgcaatctgaaattggtgctttgttggccgttgcggataaacttgatagcattgtgacgttttttgctgctggattaataccaagtggttctaatgatccttatggcttacgacgtgcagctactggcatcgtgcgtacattggtggataaaaaatggcatattgatttgcggcctttgctagctgattttgtgcaacagcaaggtaaggtaactgacaccgatttaacgacatttgttgatttcatgttggatcgtgttcgtaaattatcgttggatgctggaatacgtcaagatattgtcattgctggattaggcaacgttgatagagctgatatcgtatatattagtcagcgagtcgaagttttgtcccaacatagtggtgatggcaatttccgagatgtaattgaggcactgactcgtgtggatcgcttagccgtaaagcaagtaactaatgcaacggttgatcctgctaagtttgaaaatcaatctgaaaaggacctatatcaagcaacgttaacgcttgatttaaatactttgatgcatgacggtgcagaaaatctctacatggccttagcaaatttgcaaaaaccaattgcggcttattttgatgaaaccatggttaacgctgaagatgaatctgttaaagataatcgatatgcgcagctgaacgtcatacaacgactaaccaacggattaggagatttgacgcaaatcgtcattaagtaa

SEQ ID NO: 82 - DNA, 1347 bp, unidentified
  source 1..1347: mol_type = genomic DNA; note = DP3 Glutamine synthetase sequence
atggctcgtaaaacatttaccaaagaagaaattaaacaaattgttgttgatgaaaatgtagaattcattcgtgtaacattcactgatgtcttaggtgcgattaaaaacgttgaagtaccaacttctcaattagataaggtgcttgacaacaatttaatgtttgacggttcatcaatcgagggatttgttcgtatcaatgaatcagatatgtatctttaccccgatttatcaacatttatgattttcccatgggcaacggatggtcatggtggtaaagtggcccgcttgattgccgacatttatactgctgatcgtgagccatttgctggagacccccgtcatgcgttacgttcggtactcgctgacgcgcgtgaagctgggtttacggcgtttaatgtcgggacagaacctgaatttttcttgtttaaacttgatgaaaaaggcaacccaaccacagagttaaacgacaaaggtggttattttgacctagcaccattggatatgggtgaaaatgttcgtcgtgaaattgttttgactttggaaaaaatgggctttgaaattgaagctgctcaccacgaagttgccgaaggacagcatgaagtagactttaaatacgcttcagctcttgaagccgctgacaacattcagacgtttaagttggttgttaaaaccatcgcacgcaagaatggttactatgctacctttatgccaaagcctgttgcaggtattaacggatccggtatgcacacaaacatgtcattatttacaaaagatggtaacgcatttgttgatacatcggatgaaatgggcttgtcaaaaacagcatataacttcttgggtggtattttagaacatgcgactgcgtttacagcgcttgcaaacccaacagttaactcatacaagcgcttgacaccaggattcgaagcacctgtttatgttgcatggtcagcatcaaatcgttcaccaatggttcgagttccggcctcacgtggtaattcaacacgtttggaacttcgttcagttgacccaacagctaatccttatactgcattggcagccattttggcttcaggactggatgggatcaagcgtgaattagagcctttggcctcagttgataaaaatatttatttgatggatgaggtcgaacgggaaaaggcaggcattacagacttaccagatactctgttggctgcagttcgtgagttggcggctgatgatgttgttcgttcagctattggagaacatattgctgataagtttattgaagcaaagaagattgaatacacatcatatcgtcagtttgtttctgaatgggaaacagattcttatcttgaaaattactaa

SEQ ID NO: 83 - DNA, 2049 bp, unidentified
  source 1..2049: mol_type = genomic DNA; note = DP3 DNA gyrase subunit B sequence
gtgttcgcagattatatctgttcacacgctaataatatggcagagaatatcgaaaatgaagcattggagaacattgatggcatcgtaaccgatgataccgaaatccgtcaagcaagcaccgttcatgcagcagcaggcgcttacaatgctgatcagattcaagttttggaaggattggaagctgtccgcaaacgccctggcatgtacattggtacgaccacagcgcaaggcttgcaccatttggtatgggaaattgttgataacgggattgatgaggcattagcagggtttgcgtcacatattacggtcacaatcgaaaaggataactcaatcacggtaaccgatgacggccgtggtattcctgtcgacattcaaactaaaacgggtaagccagctcttgaaactgtctttacggtattacacgccggtggtaaatttggcggtggcggttataaagtatctggtggattacacggtgttggagcttctgttgtcaatgccttgtcaacggatttggacgttagagttgttcgtgataatactgtttattacatggacttcaaagtgggacgcgtcaacacaccgatgaaacaattgacggaaaagcccactattgagcgtggtacaattgttcattttaagcccgatgcagatattttccgtgaaacaacagtttataactacaacacattactaacacgtgtgcgcgaattggcctttttgaataaaggtttgcgcatttcgattacagataatcgacctgaagaagctgtttctgaaagctttcattttgaaggtgggattaaagaatacgtcagctatttgaataaggacaagactgctattttccctgaacctgtttacgttgagggtgaagaaaatggcattgtagtggaagctgccttacagtacactaccgatattaaagacaatctgcggacgtttactaacaatatcaatacctatgaaggtgggacgcacgaaactggctttaaaacagccttaacacgtgtaatcaatgattacgctcgtaaaaatggtcagctcaaagataatgcagaaagtttgacaggggaagatgtgcgcgaaggcatgactgctatcgtgtcaatcaagcacccagatccacaatttgaaggacaaaccaaaactaaattaggtaactccgatgcacgtcaagcaacggatcggatgttctcagaaacgttcagtcgtttcatgatggaaaatccagcagttgccaagcaaattgttgaaaaaggtgtcttagcccaaaaagcacgattggctgccaagcgtgcacgcgaaatgacacgcaaacaatctggtttggaaattggtaatttgccaggtaaattagctgataatacctcaaatgatcctgaaatttcagaattatttattgttgagggtgattcagccggtggttcagctaagcaaggacgtaaccgtttgacgcaagctattttgccaattcgaggcaaaattttaaatgttgggaaagcctcattggatcgggtgttagccaacgaagaaattcgatcattgtttacagcaatgggaactggatttggtgaggactttaatgttgaaaaagccaattatcacaaagtcattattatgacagatgccgatgtcgatggcgcccatattcgaacactattgttaacgctattttatcgttatatgcgaccacttgttgacgcaggctatatttatattgcgcagccaccgctttacggtgttgccttaggcaataataaatcaatgacgtacattgattctgatgaagaacttgaagactatttgtcacaattgccatctaatattaaaccaaaagttcaacgttataagggactaggggaaatggattacgatcaactagcagatacaaccatggatccgcagaatcgtcgtttgctacgtgttgacccaactgatgctgaagaagccgaagcagttattgatatgttaatgggtggggatgtaccaccacgtcgtaagtttattgaagacaatgctgtctttgttgagaacttggatatttaa

SEQ ID NO: 84 - DNA, 375 bp, unidentified
  source 1..375: mol_type = genomic DNA; note = DP3 Leucine--tRNA ligase sequence
atgattttcgtcaacgaagcttacaaaaccgatgctgtgccgaaagcggcggcggaaaacttcgtacagatgctgtccccactggcaccgcatttggcagaagaactgtgggaacgacttggtcataccgatacgattacgtatgaaccatggccaacgtacgatgaggcttggaccatagaatccgaagtggaaatcgtcgtgcaagtgaacggcaaaatcgtagaacgcacgaaaatttccaaagacctggatcaagcagcgatgcaagaacacagcttaagcctgccgaatgttcagcaggctgtggctgggaagacgatccgcaaagtgattgcggtgccaggcaagctggtgaatatcgtcgttggataa

SEQ ID NO: 85 - DNA, 1353 bp, unidentified
  source 1..1353: mol_type = genomic DNA; note = DP3 Glucose-6-phosphate isomerase sequence
atggcacacattacatttgacacaaagaacattgagaattttgttgcaccatacgaattggacgaaatgcaaccattaattacgatggctgaccaacaattgcgcaatcgtacgggcgctggtgcagaatattctgattggttgactctacctactgattacgacaaggaagaatttgcacgtattcaaaaggcggcgcaacaaattcaatctgattcaaagattttggttgtcattggtattggtggttcatatttgggcgcgaagatggcggttgatttcttgaatccaatgtttaataatgaattgtcggatgaccaacgtcaaggtgttaaaatttattttgctggtaactcaacttctgcagcttacttaaatgatttagttcgtgtcattggtgatcaagacttttctgtcaacgttatctcaaagtctggcacaacaacggaaccatcaatcgctttccgtgtgtttaaacaattgttagagaaaaagtatggttctgatgctgctaagaagcgtatctatgccacaacagatgccaatcgtggtgctttgcacgatgaagcagcggcttcaggttatgaaacattcacaattcctgatggtgtcggtggtcgcttctctgttttgacagctgttggcttgttgccaattgctgcttcaggcgctgatatccaaaaattgatggacggcgctcgtgatgcgcaaaacgaatatactgattctgatttgaaaaagaacgaggcatataaatatgcagccgttcgtcgtattttgtatgataagggttatacaacagaattgttgattaactgggaaccttcaatgcaatatttgtcagagtggtggaagcaattgatgggcgagtctgaaggtaaaaatcaaaagggtatctatccatcttcagctaacttctcaaccgacttgcactcacttggacaatatattcaagaaggacgccgtgatttgtttgagacggtggttaagttagacaatcctgtatctaatttggacctaccacatgaagaaggcaacaatgatggtttgcaatatttggaaggtatcacgatcgatgaagtgaacaccaaagcatctcaaggggttactttggctcacgttgatggtggtgtgcctaacttggctgttcacttgccagcacaagatgcttattcactcggttacatgatttacttctttgaaatggctgttggggcgtctggttatacgtttggtattaacccattcaaccaaccgggtgtcgaagcctataagacagctatgtttgcactattaggtaagcctggctatgaggaagcgacaaaagcattccgtgcccgcttagacaaataa

SEQ ID NO: 86 - DNA, 672 bp, unidentified
  source 1..672: mol_type = genomic DNA; note = DP3 Beta-phosphoglucomutase sequence
atgactaaattttcagatattaaaggttttgcctttgatttagatggggttattgctgatacggcgcgtttccatggtgaagcttggcatcaaacagctgatgaggttggcacaacttggacaccagaattggctgaaggtttgaagggcattagtcgtatggcttccttgcaaatgattttggatgctggggatcatgccgatgatttttcgcaagcagataaagaagcattagcagaaaagaaaaatcataattatcaacaacttatttcaacattgacggaagatgatattttgcctggcatgaaagattttattcaatcagccaaggcagccggctatacaatgtcggtggcatcagcttctaaaaacgcaccaatgattctagatcatttgggattgaccaagtattttgtcggcattgttgatcccgccactttgacaaagggaaaacctgatcctgaaatcttcgttcgtgctgcggaagtcttacatttaaatccagaaaatgttattggattggaagattcagctgctggtattgtgtcaatcaatggcgcaggtgagacatcactagccattggtaacgcagatgttttgtcaggagcggacttgaattttgcgtctacttcagaagtgaccttagcaaatattgaagctaaaatgcaatag

SEQ ID NO: 87 - DNA, 1377 bp, unidentified
  source 1..1377: mol_type = genomic DNA; note = DP3 2-oxoglutarate carboxylase small subunit sequence
atgtttaaaaaagtgcttgttgctaatcgtggtgaaattgcggttcgcatcattcgaacgctcaaagaaatggggattgcttcagtcgctatttactcgacagccgataaagatagtttacacgtacaaatcgctgacgaagcgattgctgtggggggaccgaaacctaaagattcatacttaaatatgaaaaatattttaagtgcagccctgctgtcgggagcagaggcaattcatccaggatatggctttttagctgaaaatacattgtttgctgaaatggttggcgaagttggtattaaatggattgggcctaggccagaaacaattgagttaatgggtaacaaagctaacgcacgtgaagaaatgcggcgtgccggcgtaccagtaattccaggttcagagggatttatccgtgattttcatgaagcaaaaacggttgctgataaaattggctatcctttgttgctaaaagctgccgctggtggtggtggtaaaggcatgcgttttgtttacggtgaggatgagttatcagataaatttgatgatgctcaaaacgaagcgcgtgcttcgtttggcgatgatcacatgtatattgaaaaagttatgtcacgtgttcgccacattgaaatgcaagtgtttcgtgatgagaatggtcatgttgtttacttgccagaacgaaattgctcattgcaacgcaataatcaaaaggtgattgaagaatcaccagctacgggtgtaacgcctgaaatgcgtgcgcatcttggcgaaattgttactaaagccgcaaaagcattggcgtatgaaaatactggaaccattgaatttttgcaagatcgcgatggtcatttctactttatggaaatgaacacacgtattcaagtagaacatccagtttctgaaatggtaacgggattagatttaattaagttacaaattcaagttgctgcaggcttagatttaccggtggttcaagatgacgtgatcgttcaaggccactctatcgaagtacgtttgacggctgagcagccagaaaaacactttgcacctagtgctggaacgattgattttgtttttttgccaactggtggaccgggtgttcgtattgattcagccttatttaatggcgataaaattcaaccattttacgattctatgattggcaaattaattgttaaggccgatgatcgtgaaacagccatgagaaagattcaacgtgtggttgatgaaactgttgtacgtggtgtagcaacgagccgtaattttcaaaaagctctgttagctgatccacaggttcaacgtggcgaatttgacacacgttatttggaaactgaatttttaccgagatggacacaaacattgccagataatcaataa

SEQ ID NO: 88 - DNA, 1701 bp, unidentified
  source 1..1701: mol_type = genomic DNA; note = DP1 Glutamine--tRNA ligase sequence
atgagcaagcccactgtcgaccctacctcgaattccaaggccggacctgccgtcccggtcaatttcctgcgcccgatcatccaggcggacctggattcgggcaagcatacgcagatcgtcacccgcttcccgccagagcccaacggctacctgcacatcggtcatgccaagtcgatttgtgtgaacttcggcctggctcaggagttcggtggcgttacgcacctgcgtttcgacgacaccaacccggccaaggaagaccaggaatacatcgacgccatcgaaagcgacatcaagtggctgggcttcgaatggtccggtgaagtgcgctatgcatccaagtatttcgaccagctgttcgactgggccgtcgagttgatcaaggccggcaaggcctacgttgacgacctgacccccgagcaagccaaggaataccgtggcagcctgaccgagccgggcaagaacagcccgttccgcgaccgttcggtcgaagagaacctcgactggttcaaccgcatgcgcgccggtgagttcccggacggcgcccgcgtgctgcgcgccaagatcgacatggcctcgccgaacatgaacctgcgcgacccgatcatgtaccgcattcgccatgcccatcaccaccagaccggtgacaagtggtgcatctaccccaactacgacttcacccacggtcagtcggacgccatcgaaggcatcacccactccatctgcaccctggagttcgaaagccatcgccctctgtacgaatggttcctggacagcctgccggtgccggcgcacccgcgtcagtacgaattcagccgcctgaacctgaactacaccatcaccagcaagcgcaagctcaagcaactggtcgatgaaaagcacgtgcatggctgggacgacccgcgcatgtcgacgctctcgggtttccgtcgtcgtggctacaccccggcgtcgatccgcaatttctgcgacatggtcggcaccaaccgttctgacggtgtggtcgattacggcatgcttgagttcagcatccgtcaggatctggacgcgaacgcgccgcgcgccatgtgcgtgctgcgtccgttgaaagtcgtgatcaccaactacccggaagacaaggtcgaccaccttgagctgccgcgtcacccgcagaaagaagagctgggcgtgcgcaagctgccgttcgcgcgcgaaatctacatcgaccgtgacgacttcatggaagagccgccgaagggttacaagcgcctggagccgaacggcgaagtgcgcctgcgtggcagctacgtgatccgcgccgacgaagcaatcaaggacgccgaaggcaacatcgtcgaactgcgctgctcgtacgatccggaaacactcggcaagaaccctgaaggccgtaaggtcaagggcgtgatccactgggtgccggccgctgccagcatcgagtgcgaagtgcgtctgtacgatcgtctgttccgatcgccgaacccggagaaggccgaagacagcgccagcttcctggacaacatcaaccctgactcgctgcaagtgcttacaggttgtcgtgctgagccatcgcttggcgacgcacagccggaagaccgtttccagttcgagcgcgaaggttacttctgcgcggatatcaaggactcgaaacccggtgctccggtattcaaccgtaccgtgaccttgcgtgattcgtggggccagtga

SEQ ID NO: 89 - DNA, 2418 bp, unidentified
  source 1..2418: mol_type = genomic DNA; note = DP1 DNA gyrase subunit B sequence
atgagcgaagaaaacacgtacgactcgaccagcattaaagtgctgaaaggtttggatgccgtacgcaaacgtcccggtatgtacatcggcgacaccgatgatggtagcggtctgcaccacatggtgttcgaggtggtcgacaactccatcgacgaagctttggccggtcactgcgacgacatcagcattatcatccacccggatgagtccatcacggtgcgcgacaacggtcgcggcattccggtcgatgtgcacaaagaagaaggcgtttcggcggctgaggtcatcatgaccgtgctgcacgccggcggtaagttcgatgacaactcttataaagtctccggcggtctgcacggtgtaggtgtgtcggtagtgaacgcactgtccgaagagctgatcctgaccgttcgccgtagcggcaagatttgggagcagacgtacgtccatggtgtgccacaagagccgatgaaaatcgttggcgacagtgaatccacgggtacgcagatccacttcaagccatcggctgaaaccttcaagaacatccactttagctgggacatcctggccaagcggattcgcgaactgtccttcctcaactccggtgtgggtatcgtcctcaaggacgagcgcagcggcaaggaagaactgttcaagtacgaaggcggtctgcgcgcgttcgttgaatacctgaacaccaataagaccgcggtcaaccaggtgttccacttcaacattcagcgtgaagacggcatcggcgtggaaatcgccctgcagtggaacgacagcttcaacgagaacttgttgtgcttcaccaacaacattccacagcgcgatggcggtactcacttggtgggtttccgttccgcactgacgcgtaacctgaacacttacatcgaagccgaaggcttggccaagaagcacaaagtcgccaccaccggtgacgatgcgcgtgaaggcctgaccgcgattatctcggtgaaagtgccggatcccaagttcagctcccagaccaaagacaagctggtttcttccgaggtgaagaccgccgtggaacaggagatgggcaagtacttctccgacttcctgctggagaacccgaacgaagccaagctggtcgtcggcaagatgatcgacgctgcacgtgctcgcgaagcggcgcgtaaagcccgtgagatgacccgtcgtaaaggcgcgctggatattgctggcttgcctggcaagttggctgactgccaggagaaggacccagcgctctccgagctatatcttgtggaaggtgactctgctggcggttccgccaagcagggtcgtaaccgtcgcacccaggcgatcctgccgttgaaaggcaagattctcaacgtagagaaggcccgcttcgacaagatgatttcctcccaggaagtcggcaccttgattacggcgttgggttgcggcattggccgcgatgagtacaacatcgacaagctgcgctaccacaacatcatcatcatgaccgatgctgacgtcgacggttcgcacatccgtaccttgctgctgaccttcttcttccgtcagttgcctgagctgattgagcgtggctacatctatatcgcgcagccgccgttgtacaaagtgaaaaagggcaagcaagagcagtacatcaaagacgacgacgccatggaagagtacatgacgcagtcggccctggaagatgcaagcctgcacttgaacgacgaagcaccgggtatctccggtgaggcgttggagcgtctggttaacgacttccgtatggtgatgaagaccctcaagcgtctatcgcgtctgtaccctcaggaactgaccgagcacttcatctacctgccggccgtcagtctggagcagttgggtgatcatgcagcgatgcaagagtggctggctcagtacgaagtacgcctgcgcactgttgagaagtctggcctggtgtacaaagccagtctgcgtgaagaccgtgaacgtaacgtgtggctgccggaggttgagttgatctcccacggcctgtcgaattacgtcaccttcaaccgcgacttcttcggcagtaatgactacaagacggtcgtgaccctcggcgcgcagttgagcaccttgctggatgatggtgcttacattcaacgtggcgagcgtaagaaagcggtcaaggagttcaaggaagccttggactggctgatggcggaaagcaccaagcgtcataccattcagcgatacaaaggtctgggcgagatgaaccctgatcagttgtgggaaaccaccatggatccagcacagcgtcgcatgctgcgcgtgaccatcgaagacgccattggcgcagatcagatcttcaacaccctgatgggtgatgcggtcgaacctcgccgtgacttcatcgagagcaatgccttggcggtgtccaacctggacttctga

SEQ ID NO: 90 - DNA, 1632 bp, unidentified
  source 1..1632: mol_type = genomic DNA; note = DP1 Isoleucine--tRNA ligase sequence
atgaccgactataaagccacgctaaaccttccggacaccgccttcccaatgaaggccggcctgccacagcgcgaaccgcagatcctgcagcgctgggacagtattggcctgtacggaaagttgcgcgaaattggcaaggatcgtccgaagttcgtcctgcacgacggccctccttatgccaacggcacgattcacatcggtcatgcgctgaacaaaattctcaaggacatgatcctgcgctcgaaaaccctgtcgggttttgacgcgccgtatgtcccgggctgggactgccatggcctgccgatcgaacacaaagtcgaagtgacctacggcaaaaacctgggcgcggataaaacccgcgaactgtgccgtgcctacgccactgagcagatcgaagggcagaagtccgaattcatccgcctgggcgtgctgggcgagtgggacaacccgtacaagaccatgaacttcaagaacgaggccggtgaaatccgtgccttggctgaaatcgtcaaaggcggttttgtgttcaagggcctcaagcccgtgaactggtgcttcgactgcggttcggccctggctgaggcggaagtcgaatacgaagacaagaagtcctcgaccatcgacgtggccttcccgatcgccgacgacgccaagttggcccaggctttcggcctggcaagcctgagcaagccggcggccatcgtgatctggaccaccaccccgtggaccatcccggccaaccaggcgctgaacgtgcacccggaattcacctacgccctggtggacgtcggtgatcgcctgctggtgctggccgaggaaatggtcgaggcctgtctggcgcgctacgaactgcaaggttcggtgatcgccaccaccaccggctccgcgctggaactgatcaacttccgtcacccgttctatgaccgcctgtcgccggtttacctggctgactacgtcgaactgggttcgggtacgggtgtggttcactccgcaccggcctacggcgttgacgacttcgtgacctgcaaagcctacggtatggtcaacgatgacatcctcaacccggtgcagagcaatggtgtgtacgcgccatcgctggagttcttcggcggccagttcatcttcaaggctaacgagccgatcatcgacaaactgcgtgaagtcggtgcgctgctgcacaccgaaaccatcaagcacagctacatgcactgctggcgccacaaaaccccgctgatctaccgcgccaccgcgcagtggtttatcggcatggacaaagagccgaccagcggcgacaccctgcgtgtgcgctcgctcaaagccatcgaagacaccaagttcgtcccggcctggggccaggcgcgcctgcactcgatgatcgccaatcgtccggactggtgcatctcccgccagcgtaactggggcgtaccgatcccgttcttcctgaacaaggaaagcggcgagctgcacccacgcaccgtcgagctgatggaagccgtggccttgcgcgttgaacaggaaggcatcgaagcctggttcaagctggacgccgccgagctgctgggcgacgaagcgccgctgtacgacaagaaggctcggaccaacaccgtggctggttccactcgtcgctgctga

SEQ ID NO: 91 - DNA, 1784 bp, unidentified
  source 1..1784: mol_type = genomic DNA; note = DP1 NADH-quinone oxidoreductase subunit C/D sequence
atgactacaggcagtgctctgtacatcccgccttataaggcagacgaccaggatgtggttgtcgaactcaataaccgttttggccctgacgcctttaccgcccaggccacacgtaccggcatgccggtgctgtgggtggcgcgcgccaggctcgtcgaagtcctgaccttcctgcgcaacctgcccaagccgtacgtcatgctctatgacctgcatggcgtggacgagcgtctgcggaccaagcgccagggcctgccgagcggcgccgatttcaccgtgttctatcacctgctgtcgatcgaacgtaacagcgacgtgatgatcaaggtcgccctctccgaaagcgacctgagcgtcccgaccgtgaccggcatctggcccaacgccagttggtacgagcgtgaagtctgggacatgttcggtatcgacttccctggccacccgcacctgacgcgcatcatgatgccgccgacctgggaaggtcacccgctgcgcaaggacttccctgcgcgcgccaccgaattcgacccgttcagcctgaacctcgccaagcaacagcttgaagaagaggctgcacgcttccggccggaagactggggcatgaaacgctccggcaccaacgaggactacatgttcctcaacctgggcccgaaccacccttcggcgcacggtgccttccgtatcatcctgcaactggacggcgaagaaatcgtcgactgcgtgccggacatcggttaccaccaccgtggtgccgagaagatggccgagcgccagtcgtggcacagcttcatcccgtacaccgaccgtatcgactacctcggcggcgtgatgaacaatctgccgtacgtgctctcggtcgagaagctggccggtatcaaggtgccggaccgcgtcgacaccatccgcatcatgatggccgagttcttccggatcaccagccacctgctgttcctgggtacctacatccaggacgtcggcgccatgaccccggtgttcttcaccttcaccgaccgtcagcgcgcctacaaggtcatcgaagccatcaccggcttccgcctgcacccggcctggtaccgcatcggcggtgtcgcgcacgacctgccaaatggctgggaacgcctggtcaaggaattcatcgactggatgcccaagcgtctggacgagtaccagaaagccgccctggacaacagcatcctcaagggccggaccattggggtcgcggcctacaacaccaaagaggccctggaatggggcgtcaccggtgctggcctgcgttccaccggttgcgatttcgacctgcgtaaagcgcgcccgtactccggctacgagaacttcgaattcgaagtgccgttggcggccaatggcgatgcctacgaccgttgcatcgtgcgcgtcgaagaaatgcgccagagcctgaagatcatcgagcaatgcatgcgcaacatccggcaggcccgtacaaggcggaccacccgctgaccacgccgccgccgaaagagcgcacgctgcaacacatcgaaaccctgatcacgcacttcctgcaggtttcgtggggcccggtgatgccggccaacgaatccttccagatgatcgaagcgaccaagggtatcaacagttattacctgacgagcgatggcggcaccatgagctaccgcacccggattcgcactccaagcttcccgcacctgcagcagatcccttcggtgatcaaaggtgaaatggtcgcggacttgattgcgtacctgggtagtatcgatttcgttatggccgacgtggaccgctaa

SEQ ID NO: 92 - DNA, 1059 bp, unidentified
  source 1..1059: mol_type = genomic DNA; note = DP1 Protein RecA sequence
atggacgacaacaagaagaaagccttggctgcggccctgggtcagatcgaacgtcaattcggcaagggtgccgtaatgcgtatgggcgatcacgaccgtcaggcgatcccggctatttccactggctctctgggtctggacatcgcactcggcattggcggcctgccaaaaggccgtatcgttgaaatctacggccctgaatcttccggtaaaaccaccctgaccctgtcggtgattgcccaggcgcaaaaaatgggcgccacttgtgcgttcgtcgatgccgagcacgctcttgaccctgaatacgccggcaagctgggcgtcaacgttgacgacctgctggtttcccaaccggacaccggtgagcaagccttggaaatcaccgacatgctggtgcgctccaacgccatcgacgtgatcgtggtcgactccgtggctgccctggtgccgaaagctgaaatcgaaggcgaaatgggcgacatgcacgtgggcctgcaagcccgtctgatgtcccaggcgctgcgtaaaatcaccggtaacatcaagaacgccaactgcctggtgatcttcatcaaccagatccgtatgaagattggcgtgatgttcggcagcccggaaaccaccaccggtggtaacgcgttgaagttctacgcttcggtccgtctggatatccgccgtactggcgcggtgaaggaaggcgacgaggtggtgggtagcgaaacccgcgttaaagttgtgaagaacaaggtggccccgccattccgtcaggctgagttccagattctctacggcaagggtatctacctgaacggcgagatgatcgacctgggcgtactgcacggtttcgtcgagaagtccggtgcctggtatgcctacaacggcagcaagatcggtcagggcaaggccaactcggccaagttcctggcggacaacccggatatcgctgccacgcttgagaagcagattcgcgacaagctgctgaccccggcaccagacgtgaaagctgctgccaaccgcgagccggttgaagaagtagaagaagtcgacactgacatctga

SEQ ID NO: 93 - DNA, 1161 bp, unidentified
  source 1..1161: mol_type = genomic DNA; note = DP1 RNA polymerase sigma factor RpoD sequence
atggaaatcacccgcaaggctctgaaaaagcacggtcgcggcaacaagctggcaattgccgagctggtggccctggctgagctgttcatgccaatcaagctggtgccgaagcaatttgaaggcctggttgagcgtgtgcgcagtgctcttgagcgtctgcgtgcccaagagcgcgcaatcatgcagctctgcgtacgtgatgcacgcatgccgcgtgccgacttcctgcgccagttcccgggcaacgaagtggatgaaagctggaccgacgcactggccaaaggcaaggcgaagtacgccgaagccattggtcgcctgcagccggacatcatccgttgccagcagaagctgaccgcgcttcaaaccgaaaccggtctgacgattgctgagatcaaggacatcaaccgtcgcatgtcgatcggtgaggccaaggcccgccgcgcgaagaaagagatggttgaagcgaacttgcgtctggtgatctccatcgccaagaagtacaccaaccgtggcctgcaattcctcgatctgatccaggaaggcaacatcggcttgatgaaggctgtggacaagttcgaataccgtcgcggctacaagttctcgacttatgccacctggtggatccgtcaggcgatcactcgctcgatcgcagaccaggcccgcaccatccgtattccggtgcacatgatcgagaccatcaacaagctcaaccgtatttcccggcagatgttgcaggaaatgggtcgcgaaccgacgccggaagagctgggcgaacgcatggaaatgcctgaggataaaatccgtaaggtattgaagatcgctaaagagccgatctccatggaaacgccgattggtgatgacgaagactcccatctgggtgacttcatcgaagactcgaccatgcagtcgcccatcgatgtggctaccgttgagagccttaaagaagcgactcgcgacgtactgtccggcctcactgcccgtgaagccaaggtactgcgcatgcgtttcggcatcgacatgaataccgaccacacccttgaggaagtcggtaagcagtttgacgtgacccgtgaacggatccgtcagatcgaagccaaggcactgcgcaagttgcgccacccgacgcgaagcgagcatctacgctccttcctcgacgagtga

SEQ ID NO: 94 - DNA, 4074 bp, unidentified
  source 1..4074: mol_type = genomic DNA; note = DP1 DNA-directed RNA polymerase subunit beta sequence
atggcttactcatatactgagaaaaaacgtatccgcaaggactttagcaagttgccggacgtcatggatgtcccgtaccttctggctatccagctggattcgtatcgtgaattcttgcaagcgggagcgactaaagatcagttccgcgacgtgggcctgcatgcggccttcaaatccgttttcccgatcatcagctactccggcaatgctgcgctggagtacgtgggttatcgcctgggcgaaccggcatttgatgtcaaagaatgcgtgttgcgcggtgttacgtacgccgtacctttgcgggtaaaagtccgtctgatcattttcgacaaagaatcgtcgaacaaagcgatcaaggacatcaaagagcaagaagtctacatgggcgaaatcccattgatgactgaaaacggtaccttcgttatcaacggtaccgagcgcgttatcgtttcccagctgcaccgttccccgggcgtgttcttcgaccacgaccgcggcaagacgcacagctccggtaagctcctgtactccgcgcggatcattccgtaccgcggctcgtggttggacttcgagttcgacccgaaagactgcgtgttcgtgcgtatcgaccgtcgtcgtaagctgccggcctcggtactgctgcgcgcgctcggctataccactgagcaagtgcttgatgctttctacaccaccaacgtattcagcctgaaggatgaaaccctcagcctggaactgattgcttcgcgtctgcgtggtgaaattgccgtcctggatatccaggatgaaaacggcaaggtcatcgttgaagctggccgccgtattaccgcgcgccacatcaaccagatcgaaaaagccggtatcaagtcgctggacgtgccgctggactacgtcctgggtcgcaccactgccaaggtcatcgttcacccggctacaggcgaaatcctggctgagtgcaacaccgagctgaacaccgagatcctggcaaaaatcgccaaggcccaggttgttcgcatcgagaccctgtacaccaacgacatcgactgcggtccgttcatctccgacacgctgaagatcgactccaccagcaaccaattggaagcgctggtcgagatctatcgcatgatgcgtcctggtgagccaccgaccaaagacgctgccgagaccctgttcaacaacctgttcttcagccctgagcgctatgacctgtctgcggtcggccggatgaagttcaaccgtcgtatcggtcgtaccgagatcgaaggttcgggcgtgctgtgcaaggaagacatcgtcgcggtactgaagaccttggtcgacatccgtaacggtaaaggcatcgtcgatgacatcgaccacttgggtaaccgtcgtgttcgctgcgtaggcgaaatggccgagaaccagttccgcgttggcctggtacgtgttgagcgtgcggtcaaagagcgtctgtcgatggctgaaagcgaaggcctgatgccgcaagatctgatcaacgccaagccagtggctgcggcggtgaaagagttcttcggttccagccagctctcgcagttcatggaccagaacaacccgctctccgagatcacccacaagcgccgtgtttccgcactgggcccgggcggtctgacccgtgagcgtgcaggctttgaagttcgtgacgtacacccaacgcactacggtcgtgtttgcccgatcgaaacgccggaaggtccgaacatcggtctgatcaactcccttgccgcttatgcacgcactaaccagtacggcttcctcgagagcccgtaccgtgtagtgaaagatgcactggtcaccgacgagatcgtgttcctgtccgccatcgaagaagccgatcacgtgatcgctcaggcttcggccacgatgaacgacaagaaagtcctgatcgacgagctggtagctgttcgtcacttgaacgagttcaccgttaaggcgccggaagacgtcaccttgatggacgtttcgccgaagcaggtagtttcggttgcagcgtcgctgatcccgttcctggagcacgatgacgccaaccgtgcgttgatgggttccaacatgcagcgtcaagctgtacccaccctgcgtgccgacaagccgctggtaggtaccggcatggagcgtaacgtagcccgtgactccggcgtttgcgtcgtggctcgtcgtggcggcgtgatcgactctgttgatgccagccgtatcgtggttcgtgttgccgatgacgaagttgagactggcgaagccggtgtcgacatctacaacctgaccaaatacacccgctcgaaccagaacacctgcatcaaccagcgcccgctggtgagcaagggtgatcgcgttcagcgtagcgacatcatggccgacggcccgtccaccgatatgggtgagctggcactgggtcagaacatgcgcatcgcgttcatggcatggaacggcttcaacttcgaagactccatctgcctgtccgagcgtgttgttcaagaagaccgcttcaccacgatccacattcaggagctgacctgtgtggcgcgtgacaccaagcttgggccagaggaaatcactgcagacatcccgaacgtgggtgaagctgcactgaacaaactggacgaagccggtatcgtttacgtaggtgctgaagttggcgcaggcgacatcctggttggtaaggtcactccgaaaggcgagacccaactgactccggaagagaagctgttgcgtgccatcttcggtgaaaaagccagcgacgttaaagacacttccctgcgcgtacctaccggtaccaagggtactgtcatcgacgtacaggtcttcacccgtgacggcgttgagcgtgatgctcgtgcactgtccatcgagaagactcaactcgacgagatccgcaaggacctgaacgaagagttccgtatcgttgaaggcgcgaccttcgaacgtctgcgttccgctctggtaggccacaaggctgaaggcggcgcaggtctgaagaaaggtcaggacatcaccgacgaaatcctcgacggtcttgagcacggccagtggttcaaactgcgcatggctgaagacgctctgaacgagcagctcgagaaggcccaggcctatatcgttgatcgccgccgtctgctggacgacaagttcgaagacaagaagcgcaaactgcagcagggcgatgacctggctccaggcgtgctgaaaatcgtcaaggtttacctggcaatccgtcgccgcattcagccgggcgacaagatggccggtcgtcacggtaacaagggtgtggtctccgtgatcatgccggttgaagacatgccgcacgatgccaatggcaccccggtcgacgtcgtcctcaacccgttgggcgtaccttcgcgtatgaacgttggtcagatccttgaaacccacctgggcctcgcggccaaaggtctgggcgagaagatcaaccgtatgatcgaagagcagcgcaaggtcgcagacctgcgtaagttcctgcacgagatctacaacgagatcggcggtcgcaacgaagagctggacaccttctccgaccaggaaatcctggatctggcgaagaacctgcgcggcggcgttccaatggctaccccggtattcgacggtgccaaggaaagcgaaatcaaggccatgctgaaactggcagacctgccggaaagtggccagatgcagctgttcgacggccgtaccggcaacaagtttgagcgcccggttactgttggctacatgtacatgctgaagctgaaccacttggtagacgacaagatgcacgctcgttctaccggttcgtacagcctggttacccagcagccgctgggtggtaaggctcagttcggtggtcagcgtttcggggagatggaggtctgggcactggaagcatacggtgctgcttacactctgcaagaaatgctcacagtgaagtcggacgatgtgaacggtcggaccaagatgtacaaaaacatcgtggacggcgatcaccgtatggagccgggcatgcccgagtccttcaacgtgttgatcaaagaaattcgttccctcggcatcgatatcgatctggaaaccgaataa

SEQ ID NO: 95 - DNA, 1671 bp, unidentified
  source 1..1671: mol_type = genomic DNA; note = DP22 Glutamine--tRNA ligase sequence
atgagtgaggctgaagcccgcccaacaaattttatccgtcagattattgatgaagatctggcgaccgggaaacacaataccgttcatacccgtttcccgcctgagccaaatggctatctgcatatcggtcatgcgaaatctatctgcctgaacttcggcattgcgcaagactatcaggggcagtgcaacctgcgttttgacgataccaacccggcaaaagaagacatcgaattcgttgagtcgatcaaacacgacgtccagtggttaggtttcgactggagcggtgatattcactactcttcagactattttgatcaactgcacgcttatgcgctggaactgatcaacaaaggtctggcgtacgttgacgaactgtcaccggatcagatccgtgaataccgcggctcgctgacgtctccgggcaaaaacagcccgtaccgtgaccgttcagtggaagagaacatcgcgctgtttgagaaaatgcgtaacggtgaatttgccgaaggcgctgcctgtctgcgtgcaaaaatcgatatggcgtcgcctttcttcgtgatgcgcgatccggttctgtaccgtattaagtttgcagaacaccaccagaccggcaaaaaatggtgcatctatccgatgtacgatttcacccactgcatttccgatgcgctggaagggatcacccattcgctgtgtacgctggaattccaggacaaccgccgtctgtacgactgggttctggataacatctccattccatgccacccgcgtcagtacgagttctcccgtctgaatctcgagtactccatcatgtctaagcgtaagctgaaccagctggtgaccgagaagattgtggaaggctgggacgacccgcgtatgccgactgtttcaggtctgcgtcgtcgtggttacaccgccgcgtctatccgtgaattctgccgtcgtatcggcgtcaccaagcaagacaacaacgtcgaaatgatggcgctggaatcctgtatccgtgacgatctgaacgaaaatgcaccgcgcgccatggcggtgatcaacccggttaaagtgatcattgaaaactttaccggtgatgacgtgcagagggtgaaaatgccgaaccacccgagcaaaccggaaatgggcacccgcgaagtgccatttacccgtgagatttatatcgatcaggcagatttccgcgaagaagcgaacaagcaatacaagcgtctggtgctcggcaaagaagtgcgtctgcgcaatgcgtatgtgatcaaagcagaacgtatcgagaaagatgcagaaggcaatatcaccacgatcttctgttcttacgatatcgatacactgagcaaagatcctgccgatggccgcaaggtgaaaggcgtgatccactgggtttcggcgtcagaaggcaaaccggcggagttccgcctgtatgaccgtctgttcagcgtcgccaacccgggtcaggcagaagatttcctgaccaccatcaacccggaatctctggtgatttcccacggtttcgtggagccatcactggtggctgcacaggctgaaatcagcctgcagttcgagcgtgaaggttacttctgcgccgacagccgctactcaagcgctgaacatctggtgtttaaccgtaccgttggcctgcgcgatacctgggaaagcaaacccgtcgtgtaa

SEQ ID NO: 96 - DNA, 2415 bp, unidentified
  source 1..2415: mol_type = genomic DNA; note = DP22 DNA gyrase subunit B sequence
atgtcgaattcttatgactcctcaagtatcaaggtattaaaagggctggacgcggtgcgtaagcgccccggcatgtatatcggcgataccgatgacggcactggtctgcaccacatggtattcgaggttgtggacaacgctatcgacgaagccctcgcgggccactgtaaagagattcaggtcacgatccatgcggataactctgtgtccgtacaggatgatggtcgtggcattccgaccggtattcatgaagaagagggcgtttctgctgctcaggtcatcatgaccgttcttcacgccggcggtaaatttgacgataactcgtataaagtctccggcggtctgcatggcgtgggtgtttccgtcgttaacgccctgtcagaaaaactggaactggttatccgccgcgaaggcaaagtgcacacccagacttacgtgcatggcgaacctcaggatccgctgaaagtgattggcgatactgacgtgaccggtaccacggtacgtttctggccaagcttcaacaccttcaccaatcacactgaattcgagtatgacattctggcgaaacgcctgcgtgaactgtcattcctgaactccggcgtggcgatccgcctgctggataaacgtgatggtaaaaacgatcacttccattatgaaggcggtatcaaagctttcgtggaatatctgaacaaaaacaaaaccccaatccatccgaccgtattctatttctccacggtcaaagatgacattggcgttgaagtggcgttgcagtggaacgacggtttccaggaaaacatttactgcttcaccaacaacattccacagcgcgatggcgggactcacttagccggtttccgttcggcaatgacccgtaccctgaacgcgtacatggataaagaaggctacagcaagaaatccaaaatcagcgccaccggtgatgatgcccgtgaaggcctgattgctgtggtgtcggtgaaggtgccggatcctaagttctcttctcagaccaaagacaaactggtgtcttctgaagtgaaaacagcggttgaaacgctgatgaacgagaagctggtggattacctgatggaaaacccgtcagacgccaaaatcgttgtcggtaaaatcatcgacgcagcgcgtgcccgtgaagcagcacgtaaagcgcgtgaaatgacccgccgtaaaggcgcgctggatctggctggcttgccaggcaaactggcggactgtcaggaacgcgatccggcacattccgaactgtacttagtggaaggggactcagcgggcggctctgcaaaacaaggccgtaaccgtaagaaccaggcgattctgccgttgaaaggtaaaatcctcaacgtggagaaagcgcgcttcgacaaaatgctctcttctcaggaagtggcaacgctgattacagcactcggttgcggcattggccgtgacgaatacaacccggacaaactgcgctatcacagcatcatcatcatgaccgatgccgacgtcgatggttcgcacatccgtaccctgttgctgacattcttctaccgtcagatgcctgaaattgtagaacgtggccacgtgtttatcgcccagccgccgttgtacaaagtgaaaaaaggcaagcaggaacagtacattaaagatgacgaagcgatggatcagtatcagatttccattgcgatggacggggcaacgttacacgccaacgctcatgcgccagccctggcgggtgaaccgctggagaaactggtcgctgaacatcacagcgtgcagaaaatgattggccgcatggaacgtcgttatccgcgtgcgctgctgaataacctgatctatcagccgaccctgccgggtgcagatctggccgatcaggcgaaagtgcaggcctggatggaatcgctggtggcgcgtctcaacgagaaagagcagcacggcagttcttacagcgcgatcgtgcgtgaaaaccgcgaacatcagctgttcgaaccggttctgcgtatccgcacccacggtgttgataccgattacgatctggatgccgacttcatcaaaggcggcgaataccgcaaaatctgtgcgctgggtgaacagctgcgcggcctgatcgaagaagatgccttcatcgaacgtggcgaacgccgtcagcccgtcaccagcttcgaacaggcgctggaatggctggtgaaagagtcccgtcgtggtctgtcgattcagcgatacaaaggtctgggtgaaatgaaccctgaacagctgtgggaaaccaccatggatcctgagcaacgtcgcatgttacgtgtgaccgtgaaggatgccatcgccgctgaccagttgttcacgacgctgatgggcgatgcggttgaaccgcgccgcgcctttatcgaagagaacgccctgaaagccgccaatatcgatatctga

SEQ ID NO: 97 - DNA, 2733 bp, unidentified
  source 1..2733: mol_type = genomic DNA; note = DP22 Isoleucine--tRNA ligase sequence
atgagtgactacaagaacaccctgaatttgccggaaacagggttcccgatgcgtggcgatctggccaagcgtgaacctgacatgctgaaaaattggtatgaccaggatctgtacgggattattcgtgctgccaagaaaggcaaaaaaacctttattttgcatgacggccctccgtatgcgaacggcagcattcatattggtcactcagtaaacaaaattcttaaagacatgattatcaagtccaaaggacttgcgggctttgatgcgccgtatgtgccgggctgggattgtcatggtctgccgatcgagctgaaagtcgaacaactgatcggtaagccgggcgagaaagttacggcggcggaattccgtgaagcctgccgtaaatatgccgcagaacaggttgaaggccagaagaaagacttcatccgtctgggcgtgctgggcgactgggatcatccgtacctgacgatggatttcaaaaccgaagccaacatcatccgtgcgctgggcaaaatcatcggtaacggccacctgcataaaggcgccaagccggtgcactggtgtacagattgcggttcgtcgctggccgaagccgaagtcgaatattacgacaaagcctcgccttctattgatgtggcgttcaacgcgacggatgccgcagccgtggcagcgaaatttggcgttactgcctttaatggcccgatctcgctggttatctggaccacaacaccgtggactatgcccgctaaccgcgccatttcactgaatcctgagtttgcttatcagctggttcaggtcgaaggtcagtgtctgatcctggcaaccgatctggttgaaagcgtcatgaaacgtgccggtattgccggatggaccgttctgggcgagtgcaaaggcgcagacctcgaactgctgcgcttcaaacacccgttcctcggtttcgacgttccggcgatcctgggcgatcacgtgacgctcgatgcgggtaccggtgccgtgcataccgcaccaggccacggccctgacgactttgttatcggccagaaatacggtctggaagtggcgaatccggtagggccgaacggttgctacctgccgggcacttacccgacgctggacggtaaatttgtctttaaagccaacgacctgatcgttgagttgctgcgtgaaaaaggcgcattgctgcacgttgagaaaatcacgcacagctatccttgctgctggcgccacaaaacgccaatcatcttccgcgcgacgccgcaatggttcatcagcatggatcagaagggcctgcgtcagcagtcgctggaagagatcaaaggcgtgcagtggatcccggactggggtcaggcacgtatcgaaaacatggtcgctaaccgtcctgactggtgtatctcccgtcagcgtacctggggcgtgccgatgtctctgttcgttcacaaagacactgagcagctgcatccgcgcagccttgagctgatggaagaagtggcgaaacgtgttgaggtggatggcattcaggcgtggtgggatctgaatccggaagacattctgggtgcagacgccgcagattacgtcaaagtaccggacacgctggacgtctggtttgactccggttcaacgcattcttccgttgtggatgtgcgtcctgagttcaacgggcattctcctgatctgtatctggaaggttctgaccagcatcgcggctggttcatgtcttccctgatgatttcgacggcaatgaaaggcaaagcgccttacaaacaagtgctgactcacggtttcaccgtggatggtcagggccgcaaaatgtctaaatccatcggcaataccatcgcgccgcaagacgtgatgaacaagctgggtggcgacattctgcgtctgtgggtcgcgtcgacggattacaccggcgaaatcgccgtgtccgacgaaatcctcaaacgtgctgctgattcttaccgccgtatccgtaacaccgcgcgcttcctgctggcgaaccttaacggtttcgatccggcgctgcacagcgtggctccggaagacatggtggtgctggaccgctgggcggttggccgtgcgaaagccgctcaggaagaaatcattgctgcgtatgaagcctatgatttccatggcgttgttcagcgtctgatgcagttctgctcgatcgaaatgggttccttctatctggatatcattaaagatcgtcagtacaccgcgaaaagcgacagcgttgcacgtcgcagctgtcagaccgcgctgtatcacatcagtgaagcgctggttcgctggatggcaccgatcatgtcgttcacagccgatgaaatctgggcggaactgccgggaagccgtgagaaattcgtcttcaccgaagagtggtacgacggtctgttcggtctcgcaggcaacgaatccatgaacgatgcgttctgggatgaactgctgaaagtgcgtggcgaagtgaacaaagtgatcgaacaggcgcgtgcggataaacgtctgggcggttctctggaagcagcggttacgctgtttgctgatgatgcgctggcaacagacctgcgttctctgggcaatgaactgcgctttgtgctgctgacgtcaggggcgaaagttgccgcactgagtgatgcagatgacgcggctcagtcgagtgaattgctgaaaggcctgaagattggtctggcgaaagcagaaggcgacaagtgcccgcgctgctggcattacactaccgattaa

SEQ ID NO: 98 - DNA, 1800 bp, unidentified
  source 1..1800: mol_type = genomic DNA; note = DP22 NADH-quinone oxidoreductase subunit C/D sequence
atgacagatttgacgacgcaagattccgccctgccagcatggcatacccgtgatcatctcgatgatccggttatcggcgaattgcgtaaccgttttgggccagaggcctttactgtccaggcaacccgcaccggaattcccgtggtgtggttcaagcgtgaacagttactggaagcgattacctttttacgaaaacagccaaaaccttacgtcatgcttttcgatttgcatggctttgatgagcgtttacgtacacaccgcgacggtttaccggctgcggatttttccgttttctaccacctgatctccgtcgagcgtaaccgcgacatcatgatcaaagtggcgttgtcagaaaacgatcttcatgttccgacgatcaccaaagtgttcccgaacgctaactggtacgaacgcgaaacatgggaaatgttcggtattaccttcgacggccatccgcacctgacgcgcatcatgatgccgcagacctgggaagggcatccgctgcgtaaagactatccggcgcgcgccaccgagttcgatccttatgagctgactaagcaaaaagaagaactcgagatggaatcgctgaccttcaagccggaagactggggcatgaagcgcggtaccgataacgaggactttatgttcctcaacctcggtcctaaccacccgtcagcgcatggtgcattccgtattatcctgcagctggatggcgaagagattgtcgactgcgtgcctgacgtcggttaccaccaccgtggtgcggagaaaatgggcgaacgccagtcatggcacagctacattccgtatactgaccgtatcgaatatctcggcggttgtgttaacgaaatgccttacgtgctggctgttgaaaaactcgccggtatcgtgacgccggatcgcgttaacaccatccgtgtgatgctgtctgaactgttccgtatcaacagccatctgctgtacatctctacgtttattcaggacgtgggtgcgatgacgccggtattcttcgcctttaccgatcgtcagaaaatttacgatctggtggaagcgatcaccggtttccgtatgcacccggcctggttccgtatcggtggcgtagcgcatgacctgccgaaaggctgggaccgcctgctgcgtgaattccttgactggatgccagcccgtttggattcctacgtcaaagcggcgctgagaaacaccattctgattggccgttccaaaggcgtggccgcgtataacgccgacgacgcactggcctggggcaccaccggtgctggcctgcgcgcaacgggtatcccgttcgatgtgcgtaaatggcgtccgtattcaggttatgaaaactttgactttgaagtgccgaccggtgatggcgtcagtgactgctattcccgcgtgatgctgaaagtggaagaacttcgtcagagcctgcgcattctggaacagtgctacaaaaacatgccggaaggcccgttcaaggcggatcacccgctgaccacgccgccaccgaaagagcgcacgctgcaacacatcgagaccctgatcacgcacttcctgcaagtgtcgtgggggccggtcatgcctgcacaagaatctttccagatggttgaagcaaccaaagggatcaacagctactacctgaccagtgacggcagcaccatgagctaccgcacccgtgtccgtacgccgagcttcccgcatttgcagcagatcccgtccgtaatccgtggcagcctggtatccgacctgatcgtgtatctgggcagtatcgattttgtaatgtcagatgtggaccgctaa

SEQ ID NO: 99 - DNA, 1065 bp, unidentified
  source 1..1065: mol_type = genomic DNA; note = DP22 Protein RecA sequence
atggctattgatgagaacaagcaaaaagcgttagctgcagcactgggccagattgaaaagcaattcggtaaaggctccatcatgcgtctgggtgaagatcgctccatggacgttgaaacgatctctaccggctctttgtctctggatatcgcgttaggtgccggcggtttgccaatgggccgtatcgttgagatctatggcccggaatcttccggtaaaacaacgctgaccttgcaagttatcgcggctgcacagcgtgaaggcaaaacctgtgcgttcatcgatgcagaacacgccctggacccgatctacgctaaaaaactgggcgtggatatcgataacctgctgtgttctcagccagataccggcgaacaggctctggaaatctgtgacgcgctgacccgttcaggcgctgttgacgtgatcatcgttgactccgttgccgcactgacaccgaaagcggaaatcgaaggcgaaattggtgactctcacatgggcctcgcggcacgtatgatgagccaggcgatgcgtaagctggccggtaacctgaaaaacgccaacaccttgctgatcttcatcaaccagatccgtatgaaaattggtgtgatgttcggtaacccggaaaccaccaccggcggtaacgccctgaaattctacgcttctgtgcgtctggatatccgccgtatcggcgcgatcaaagaaggcgatgtggttgtcggtagcgaaacgcgtgtgaaagtggtgaagaacaaaatcgctgcgccatttaaacaagctgaattccagatcatgtacggcgaaggcatcaatatcaacggcgagctgattgatctcggcgtgaagcacaagctgatcgaaaaagccggtgcatggtatagctacaacggtgagaagattggtcagggtaaagcgaactcctgcaacttcctgaaagaaaacccgaaagtggctgccgagctggataaaaaactgcgtgatatgctgttgagcggtaccggtgaactgagtgctgcgaccacggctgaagatgctgacgacaacatggaaaccagcgaagagttttaa

SEQ ID NO: 100 - DNA, 1839 bp, unidentified
  source 1..1839: mol_type = genomic DNA; note = DP22 RNA polymerase sigma factor RpoD sequence
atggagcaaaacccgcagtcacagcttaagctacttgtcacccgtggtaaggagcaaggctatctgacctatgctgaggtcaatgaccatctgccggaagatatcgtcgattccgaccagatcgaagacatcatccagatgattaacgacatgggcatccaggtacttgaagaagcaccggacgccgatgatttgatgctggccgaaaaccgccctgataccgatgaagacgctgcagaagccgcggcgcaggtgctttccagcgttgaatccgaaattggccgtaccaccgaccctgtgcgtatgtatatgcgcgagatgggtaccgttgagttgctgacccgtgaaggcgaaatcgacatcgccaaacgtatcgaagacggtatcaatcaggtccagtgctccgttgctgaatatcctgaagctatcacttatttgttagagcaatatgaccgtgtggaagcaggcgaagtacgtctgtctgacctgatcaccggttttgttgacccgaacgccgaagaagaaatcgcaccaactgcgactcacgtgggttctgaactgaccactgaagagcagaatgatgacgacgaagacgaagatgaagacgacgacgctgaagacgacaacagcatcgatccggaactggctcgccagaagttcaccgaactgcgtgaacagcatgaagcgacgcgtctggtcatcaagaaaaacggccgtagtcacaagagcgcagcagaagaaatcctgaagctgtccgatgtgttcaaacagttccgtctggtgccaaaacagttcgatttcctggttaacagcatgcgttccatgatggatcgcgttcgtgctcaggaacgtctgatcatgaaagtgtgcgttgaacagtgcaaaatgccgaagaaaaacttcgtcaatctgttcgccggtaacgaaaccagcgatacctggtttgatgccgctctggcaatgggtaaaccatggtccgagaagctgaaagaagtcaccgaagacgtgcaacgcggcctgatgaaactgcgtcagatcgaagaagaaaccggcctgactatcgaacaggttaaagacatcaaccgtcgcatgtcgatcggcgaagcgaaagcccgtcgcgcgaagaaagagatggttgaagcaaacttacgtctggttatttctatcgccaagaaatacaccaaccgtggtctgcagttccttgacctgatccaggaaggtaacatcggcctgatgaaagccgttgataagtttgaatatcgccgtggttataagttctcaacttatgcgacctggtggatccgtcaggctatcacccgctccatcgccgaccaggcgcgtaccatccgtatcccggtacatatgattgagacgatcaacaaactcaaccgtatctcccgtcagatgctgcaagagatgggccgcgaaccgacaccggaagagctggctgagcgtatgttgatgccggaagacaaaatccgcaaagtgctgaaaattgccaaagagccaatctccatggaaacgccaatcggcgacgatgaagattcgcatctgggcgatttcatcgaggataccaccctcgagctgccactggattctgcgacgtctgaaagcctgcgttctgcaacgcatgacgttctggctggcctgactgcacgtgaagcgaaagttctgcgtatgcgtttcggtatcgatatgaacactgaccacacgctggaagaagtgggcaaacagttcgacgtgacccgtgagcgtatccgtcagatcgaagcgaaagcgttgcgtaaactgcgccacccgagccgctccgaagtactgcgcagcttcctggacgattaa

SEQ ID NO: 101 - DNA, 4221 bp, unidentified
  source 1..4221: mol_type = genomic DNA; note = DP22 DNA-directed RNA polymerase subunit beta' sequence
gtgaaagacttactaaagtttctgaaagcgcaaactaagaccgaagagtttgatgcgatcaaaattgctctggcatcgccagacatgatccgttcttggtcttttggtgaagttaagaagccagaaaccattaactaccgtacgttcaaaccagaacgtgacggccttttctgtgcccgtattttcggaccagtaaaagactacgaatgcctgtgcggtaagtacaagcgtttaaaacatcgcggcgtgatctgcgagaagtgcggcgttgaagtgacccagactaaagtacgccgtgagcgtatgggccacatcgaactggcttccccgactgcacacatctggttcctgaaatcgctgccatcgcgcatcggtttgctgctggatatgccactgcgtgacatcgaacgtgttctgtacttcgaatcctatgtggttatcgaaggcggcatgactaacctcgaaaaacgccagatcctgactgaagagcagtatctggatgcgttggaagagtttggtgatgagttcgacgcgaagatgggtgcggaagctattcaggccctgttgaaaaacatggatctggaagcagagtgcgagcaactgcgtgaagagttgaacgaaaccaactccgaaaccaaacgtaagaagctgaccaagcgtatcaagctgctggaagcgttcgttcagtctggtaacaaaccagagtggatgatcctgactgtgctgccggtactgccaccagacttgcgtccattggttccgttggacggcggccgtttcgcaacgtcggatctgaacgatctgtatcgtcgcgtgatcaaccgtaacaaccgtctgaaacgcctgctggatctggctgcgccagacatcatcgtacgtaacgaaaaacgtatgctgcaagaagcggtagatgctttgctggataacggccgtcgcggtcgtgctatcaccggctctaacaagcgtccgctgaaatctctggcagacatgattaaaggtaaacagggtcgtttccgtcagaacttgctgggtaaacgtgtcgactactctggtcgttccgttatcaccgtaggtccatacctgcgtctgcaccagtgtggtctgccgaagaaaatggcactggaactgttcaaaccgttcatctacggcaagctggaactgcgtggcctggccaccaccatcaaagccgcgaagaaaatggttgagcgcgaagaagctgtcgtttgggacatcctggacgaagttatccgcgaacacccggtactgctgaaccgtgcaccaaccctgcaccgtttgggtatccaggcgtttgaaccggttctgatcgaaggtaaagcaatccagctgcacccgctggtttgtgcggcatataacgccgacttcgatggtgaccagatggctgttcacgtaccgttgacgctggaagcccagctggaagcgcgtgcgttgatgatgtctaccaacaacatcctgtcacctgcgaacggcgagccaatcatcgttccttctcaggacgttgtattgggtctgtactacatgacccgtgactgtgttaacgccaaaggcgaaggcatggttctgaccggtcctaaagaagctgagcgtatttaccgcgccggtttggcctctctgcatgcgcgtgtcaaagtgcgtattacagaagagatcaaaaataccgaaggcgaagttacgcacaagacgtcgattatcgacacgacagttggtcgcgccatcctttggatgatcgtacctaaaggtctgccgttctctatcgtcaaccagcctctgggcaaaaaagctatctccaaaatgctgaacacctgttaccgcattttgggcctgaagccgaccgttatttttgctgaccagatcatgtacaccggttttgcttacgctgcccgttcaggcgcgtcagtaggtatcgatgacatggtaatccctgcgaagaaagcagagatcatcgaagaagcagaaaccgaagttgctgaaatccaggaacagttccagtctggtctggtcactgctggcgaacgctataacaaagtgatcgacatctgggctgcggccaacgaacgtgttgctaaggcaatgatggaaaacttgtctgttgaagacgtcgtcaaccgtgacggtgttgttgaacagcaggtttccttcaacagtatctttatgatggccgactccggtgcgcgtggttctgctgcacagattcgtcagctggccggtatgcgtggcctgatggcgaaaccagatggttccatcattgaaacgccaatcaccgcgaacttccgtgaaggtctgaacgtactccagtacttcatctctactcacggtgctcgtaaaggtttggcggataccgcacttaaaacggctaactccggttatctgacccgtcgtctggttgacgtcgcgcaggatctggttgtgaccgaagacgactgtgggactcacgaaggcatcatgatgactccggtcatcgaaggtggcgacgttaaagaaccactgcgtgagcgtgtactgggtcgtgtgactgcagaagatatcctcaagccgggtacggcggatatcctggttccacgtaacaccctgcttcacgagaagacgtgtgatctgttagaagagaactcagtcgacagcgtgaaagtacgttcagtcgtaagttgcgaaaccgactttggtgtgtgtgcaaactgctacggtcgcgacctggcacgtggtcacatcatcaacaaaggtgaagcgatcggtgttattgcagcacagtccatcggtgagccgggtacccagctgacgatgcgtacgttccacatcggtggtgcggcatctcgtgcggcagcggaatccagcatccaggttaagaacactggtaccattaaactgagcaaccacaagcacgttagcaactctaacggcaaactggtgatcacttcccgtaacactgagctgaaattgatcgacgaattcggtcgtaccaaagaaagctataaagtgccttacggttccgtgatgggcaaaggcgatggcgcatcagttaacggcggcgaaaccgttgctaactgggatccgcacaccatgccagttatcagtgaagtgagtggtttcattcgctttgccgatatggtggatactcagaccatcacacgccagaccgacgacctgaccggtttgtcttctctggttgttctggactctgcagagcgtaccggtagcggtaaagacctgcgtccggcactgaaaatcgttgacgctaaaggcgacgacgtattgattccaggtactgatatgcctgctcaatacttcctgccaggtaaagcgattgttcagctggaagatggtactcagatccactctggtgacaccctggcgcgtattcctcaggaatccggcggtaccaaggacatcaccggtggtctgccacgcgttgctgacctgttcgaagcacgtcgtccgaaagagcctgcaatccttgctgaaatcagcgggatcatctccttcggtaaagaaaccaaaggcaaacgtcgtctggtaatttctccgttagatggcagcgatgcttacgaagaaatgatccctaaatggcgtcagctgaacgtgttcgaaggcgaagttgtggaacgtggtgacgtcgtatccgacggccctgagtctccgcacgacatcttgcgtttacgtggtgttcacgcggttacccgctacatcaccaacgaagtgcaggaagtttaccgtctgcaaggcgttaagattaacgataagcacatcgaagttatcgttcgtcagatgttgcgtaaaggcaccatcgttagcgctggtggcactgacttcctggaaggcgagcaggcagaaatgtctcgcgttaaaatcgctaaccgtaagctggaagctgaaggcaaaatcacggcaacattcagccgtgacctgctcggtatcaccaaggcatccctggcgaccgaatccttcatctctgcagcgtcgttccaggaaaccacgcgtgttcttaccgaagcggctgttgccggtaaacgtgatgaactgcgtggcctgaaagagaacgttatcgttggccgtctgatcccagccggtaccggttacgcttatcatcaggatcgtgcacgccgtaaagcacaaggcgaagtgccagttgtaccgcaagtcagcgcggatgaagcaacggctaacctggctgaactgctgaacgcaggtttcggtaacagcgacgattaa

SEQ ID NO: 102 - DNA, 1668 bp, unidentified
  source 1..1668: mol_type = genomic DNA; note = DP67 Glutamine--tRNA ligase sequence
atgagtgaggctgaagcccgcccaactaactttattcgtcagattatcgacgaagatctggcgaacggtaagcacagttcagtgcacacccgcttcccgcctgagccgaatggctatctgcatattggccatgcgaaatcaatctgcctgaactttggtatcgctcaggattatcaggggcagtgtaacctgcgctttgatgacactaacccggtgaaagaagatctggagtttgttgaatcaatcaagcgtgatgtgcagtggctgggctttaagtggagtggtgacgtacgctactcatctgactatttcgagcaactgcacaattatgccgttgagctgattagtaaagggctggcgtacgttgatgaactgtcaccggagcagatccgtgaataccgtggcagcctgacctcagcgggtaaaaacagccccttccgcgatcgcagcgtggacgaaaaccttgcgctctttgcaaaaatgcgcgcgggcggctttgccgagggcaccgcgtgtttacgagccaaaattgatatggcttccaactttatcgttctgcgcgatccggtgatctaccgcatcaaatttgccgaacatcatcagaccggcaataagtggtgcatctatccgatgtatgactttacccactgcatctctgatgcgctggaaggcattactcactcactgtgtacgctggaattccaggataaccgtcgcctgtacgactgggtgctggataacatcaccattccggttcatccgcgtcagtatgaattctctcgcctgaatcttgaatatgccatcatgtccaagcgtaagttgagtcagttggtgaccgagaacgtggtggaaggttgggatgatccccgtatgctgactgtttcgggtttgcgccgccgtggctacactgcggaatccatccgtgaattctgccgccgcattggggtgaccaagcaggacaatattgttgaaatggccgctctggaatcctgtatccgtgacgacctcaatgagaatgccccgcgtgccatggcagtgatggatccggtaaaagtggtgatagaaaatctgcctgcgcatcacgatgaggtgatcaccatgccgaatcatccgagcaagccggaaatgggtacccgcgaagtcccgttcagtcgtgagatctacatcgatcgtgctgacttccgtgaggaagcaaacaagcagtacaagcggctggtgctgggcaaagaagtgcgtctgcgtaacgcttatgtgatcaaagccgagcgcgtggcaaaggacgatgaaggcaacattacctgcctgttctgtacctgtgatgtggatactctgagcaaggatccggccgacgggcgtaaagtgaagggcgttatccactgggtgtcagctgttcatgcccttccggcagagttccgtctgtacgatcggctgttcagcgtaccgaatccgggggcggcagaagacttcctggccagcatcaacccggaatctctggtgatccgtcagggcttcgtggagcccgggatgcagcaggcggaggcgtcagccccgtatcagtttgagcgtgaaggctacttctgcgctgacagtgtctactccagtgccagcaatctggtgttcaaccgcaccgttggcctgcgtgacacctgggcgaaagtcggcgagtaa

SEQ ID NO: 103 - DNA, 2409 bp, unidentified
  source 1..2409: mol_type = genomic DNA; note = DP67 DNA gyrase subunit B sequence
atgtcgaattcttatgactcctccagtatcaaagttctgaaagggctcgatgctgtacgcaaacgcccgggtatgtatatcggcgatacggatgacggtaccggtctgcatcacatggtatttgaggtcgtggataacgccattgacgaagcgctcgccggtcactgttccgatattcttgtcactattcatgccgataactctgtttccgttgtggatgatggccgtggtattccgaccggtattcacgaagaagaaggcatctcagccgctgaagtgatcatgaccgtgctgcacgccggcggtaagttcgacgataactcttataaagtctccggcggcctgcacggcgtgggcgtgtcagtggtgaacgccctgtcggaaaaactggagctgaccattcgtcgcgaagggaaagttcaccagcagacttacgtccacggcgtgccacaggccccgttgagtgtgagcggtgaaactgacctgacgggaacgcgcgtgcgtttctggcccagccatcagacgttcactaacgtcgtggagttcgagtacgaaattttggcaaagcgcctgcgtgagctgtcgttcctgaactccggtgtatcaatcaagctggaagataagcgcgacggtaaaagcgaccattaccactatgaaggtggtatcaaggcgtttgttgagtacctcaacaagaacaaaaccccgatccacccgaatgtgttctatttctcaaccgagaaagacggcattggtgtggaagtggcgctgcagtggaacgatggtttccaggaaaatatctactgctttaccaacaacatcccacagcgggatgggggcacgcacctcgttggtttccgtaccgcgatgacccgtaccctgaatgcctacatggataaagaaggctacagcaagaaagccaaagtcagcgccaccggtgacgacgcgcgtgaaggcctgattgctgtggtgtcggtgaaagtgccggatccgaaattctcttcacagaccaaagataaactggtctcttctgaagtgaaaaccgccgttgagcagcagatgaacgagctgctggcagaatacctgctggaaaacccgaccgatgccaaaatcgtcgtcggtaaaatcattgatgcggcccgcgcccgtgaagcggcccgtcgtgcacgtgaaatgacccgccgtaaaggcgcgctggatctggcaggcctgccgggcaaactggcggactgccaggagcgtgatccggctctgtccgaaatttacctggtggaaggggactctgcgggcggctctgccaagcagggacgtaaccgtaaaaaccaggccatcctgccgctgaagggtaaaatcctcaacgtcgagaaggcgcgctttgacaagatgctcgcgtcgcaggaagtcgctacgctgatcaccgcgctgggctgtggtatcggtcgtgatgagtacaaccccgacaaactgcgctatcacagcatcattatcatgaccgatgccgacgtggatggctcgcatatccgtaccctgctgctgaccttcttctaccgtcagatgccagaaatcattgagcgtggtcatgtctatattgcccagccaccgctgtacaaggtgaaaaaaggcaagcaggagcagtatattaaagacgacgatgcgatggatcagtaccagatcgccatcgcgctggacggtgccacgctgcatgcgaacgccagcgccccggcccttggcggtaagccactggaagatctggtgtctgagttcaacagcacgcgcaagatgatcaagcgcatggagcgccgttacccggtggccttgctgaatgcgctggtctacaacccgaccctgagcgatttgaccgccgaagcgccggtacagagctggatggatgtgctggtgaagtatctgaacgacaacgaccagcacggcagcacctacagcggtctggtacgcgaaaatctggagctgcatatctttgagccggtactgcgtatcaaaacccacggcgtggataccgattatccgctcgacagcgagtttatgctcggcggcgaataccgtaagctctgcgcgctgggtgagaagctgcgtggcctgatcgaagaagacgcgttcatcgaacgtggtgagcggcgtcagccgattgccagctttgagcaggcgatggagtggctggttaaagagtcacgccgtggcctgacggttcagcgttataaaggtctgggcgagatgaacccggatcagctgtgggaaaccaccatggatccggacagccgccgtatgctgcgcgtgaccatcaaagatgccgtggccgccgaccagctgttcaccaccctgatgggggatgcggtagagccccgtcgtgcctttattgaagagaacgccctgcgcgcggcaaacatcgatatctga

SEQ ID NO: 104 - DNA, 2817 bp, unidentified
  source 1..2817: mol_type = genomic DNA; note = DP67 Isoleucine--tRNA ligase sequence
atgagtgactataaatctaccctgaatttgccggaaacggggttcccgatgcgtggcgatctggccaaacgcgaaccgggtatgctgcaacgttggtatgatgacaagctgtacggcatcattcgcgaagccaagaaagggaaaaaaacctttatcctgcacgatggccctccttacgccaacggcagcattcatattggtcactccgttaacaagattctgaaagacattatcgttaagtcgaaaggcatggcgggctatgactcgccttatgtaccgggttgggactgccacggtctgcctatcgagcataaagttgagcagatgatcggtaagccgggagagaaagtcagcgccgctgagttccgtgctgcctgccgcaaatacgctgccgagcaggtggaagggcagaaagccgactttatccgtctgggtgtgttgggtgactgggatcgtccgtatctgacaatgaacttccagaccgaagccaatattatccgtgcgctgggtaaaatcatcggtaacgggcacctgcacaaaggggccaagccggtacactggtgcctggactgccgttctgccctggctgaggcggaagtggagtactacgataaaacctctccgtctatcgatgtcatgttcaatgcgactgataaagagggggtacaggccaaatttgcggcaacgaatgttgacggcccgatctcgctggtgatctggactaccacgccgtggaccatgccggctaaccgcgctatctcactgcatcctgaattcgactaccagctggtacagattgaaggccgtgctctgatcctcgccaaagagatggttgagagcgtgatgcagcgcgttggtgttgccgcctggaccgtgctgggcgaagcgaaaggggcagacctggagctgatgggcttccagcatccgttcctcgaccatacctctccggttgtgctgggtgagcatgtcacgctggaagccggtaccggtgcggtccataccgcaccaggccatggcccggacgactatgttatcggtcagaaatacggtatcgaagtggctaacccggtcggcccggatggctgctacctgccgggaacctacccgacgctggatggtgtgaacgtctttaaagccaacgatatgatcgttgaactgctgcgtgaaaagggtgctctgctgcacgttgagaaactgttccacagctatccacactgctggcgtcataaaacgcccatcatcttccgcgctacgccacagtggtttatcagcatggatcagaagggcctgcgtgcgcagtcgctgaaagagatcaagggcgtgcagtggatcccggactggggtcaggcacgtattgaatcgatggtcgcgaaccgtcctgactggtgtatttcccgtcagcgtacctggggcgtgccgatggcgctgttcgtccataaagacaccgaacagctgcacccggattcgctggagctgatggagaaagtggcgaagcgggttgagcaggacggcattcaggcatggtgggatcttgatgcccgcgacctgatgggcgccgatgctgacaactacgttaaagtcccggataccctggacgtctggtttgactccggttcaaccagctactcggtcgtcgatgcccgccctgaatttgacggcaatgcccctgacctgtatctggaaggatcggatcagcaccgcggctggtttatgtcctcactgatgatctcgaccgcgatgaaaggcaaagcgccttaccgtcaggtactgacgcacggcttcaccgtcgatggtcagggccgtaagatgtccaagtcactgggcaatactgtcagcccgcaggatgtgatgaacaaactgggcgccgatattctgcgcctgtgggtcgcctctacggactactccggtgagatcgccgtatccgacgagatccttaaacgctctgccgacagctatcgccgcatccgtaacaccgcacgtttcctgctggcaaaccttgccggttttaatccggaaaccgatagggtgaaaccggaagagatggtggtggtggatcgctgggccgttggccgtgcgctggcggcacagaatgatatcgtagcctcgtatgaagcttatgacttccatgaagtcgtgcagcgtctgatgcagttctgttcggttgagatgggctccttctacctggatatcatcaaggatcgtcagtacaccgcgaaggccgatggcctggcgcgtcgcagctgtcagacggcgctgtggtatatcgtggaagcgctggtgcgctggatggcaccgattatgtccttcactgccgatgaaatctggggttacctgccgggtaaacgcagccagtatgtctttaccgaagagtggtttgacgggctgttcagcctggaggacaatcagccgatgaacgacagttactgggcagaactgctgaaagtacgcggtgaagtcaacaaggtgatcgagcaggcccgcgctgataagcggattggcgggtctctggaagccagcgtgacgctgtatgctgacgcagacctggccgcgaagctgaccagcctgggtgaggagctgcgctttgtgttgctgacttccggggcgcaggttgcggattatgcgcaggccaccgctgatgcacagcaaagcgaaggggtaaaaggtctgaaaattgccctgagcaaagcggaaggcgagaagtgcccgcgctgctggcattacactaacgatatcggccagaatgctgaacacgctgacgtgtgcggccgttgtgtcactaacgtcgcgggcagcggcgaacagcgtaagtttgcatga

SEQ ID NO: 105 - DNA, 1731 bp, unidentified
  source 1..1731: mol_type = genomic DNA; note = DP67 NADH-quinone oxidoreductase subunit C/D sequence
gtgatcggcgagctgcgtaatcgttttgggcctgatgcctttacagtacaagcgacccgtaccggcgtgccggtggtctgggtaaaacgtgagcagttgcttgagattattgagttcctgcgcaagctgcctaaaccctatgtgatgctgtatgacctgcatggcatggatgagcgcctgcgtactcaccgtgccggtttaccggcggcggatttttccgttttctatcacttcatctccattgaacgtaaccgcgacatcatgctcaaggtggcgttgtctgaaaacgatttgaatgtgcccaccatcaccaaaattttcccgaatgccaactggtatgagcgtgaaacctgggagatgtttggtatcaatgttgaaggccacccgcacctgacgcgcattatgatgccgcagagctgggaagggcatccgctgcgcaaagattaccctgcgcgtgcgaccgagttcgatccgtttgaactgaccaagcagaaagaagatctggagatggaatctctgaccttcaagcctgaagactggggcatgaagcgttcgaccaacaatgaggacttcatgttcctcaacctgggcccgaaccacccttctgcgcacggcgcgttccgtatcatcctgcaactggacggtgaagagatcgtcgactgcgtgccggatatcggataccaccatcgtggtgccgaaaaaatgggtgaacgccagtcctggcacagctacattccgtataccgaccgtattgagtatctcggcggctgcgtaaacgaaatgccgtacgtgctggcggtagaaaagctggctggtatcaaagtccctgagcgcgtggaagtcattcgcgtgatgctatcagagctgttccgtataaacagccacctgctgtacatctctacgtttatccaggacgtcggtgctatgtccccggtgttctttgcctttactgaccgccagaaaatttacgacgtggtagaagccattaccggcttccgtatgcatccggcctggttccgcattggtggcgtggcgcatgatctgcctaaaggctgggagcgcctgctgcgtgagttcctggattggatgcctaagcgtctgaaagcctatgagcagaccgcactgaaaaactccgtgcttattgcccgttccaaaggggtttctgcctataacatggaagaagcactggcctggggcacgacgggggctggcctgcgtggtaccggtctggactttgatgtgcgtaaatggcgtccatattccggttatgaaaacttcgatttcgaagtgccaatcggagatggcgtaagctgtgcttacacccgtgtcatgctgaagatggaagagatgcgccagagtatgcgcatcctggaacagtgcctgaagaacatgccagcaggcccgttcaaggctgaccatccgctgaccacgccgccgccgaaagagcgcacgctgcagcatatcgaaaccctgatcactcacttcctgcaggtttcgtggggcccggtaatgccggcaaacgaatccttccagatgattgaagcgaccaaagggatcaacagttactacctgaccagtgatggcagcacgatgagctaccgcacccgcgtgcgtacgccgagcttcccgcatttgcaacagatcccatcggtgatcaacggcagcctggtatccgatctgatcgtatacctcggtagtatcgattttgttatgtcagacgtggaccgctaa

SEQ ID NO: 106 - DNA, 1062 bp, unidentified
  source 1..1062: mol_type = genomic DNA; note = DP67 Protein RecA sequence
atggctatcgacgaaaacaagcaaaaagcactggcagcagcgctgggccagattgaaaagcagtttggtaaaggctccatcatgcgcctgggtgaagaccgcaccatggatgtggaaaccatctcaaccggttctttatcactggatatcgcgctgggtgccggtggtttaccaatgggccgtatcgttgaaatctatggcccggagtcttccggtaaaaccaccctgacgctgcaggttatcgcttctgcacagcgtaaagggaaaacctgtgcatttatcgatgccgagcatgctctggacccggtctacgctaaaaaactgggcgtggatatcgataacttgctgtgttctcagccggataccggtgagcaggcgctggaaatctgtgatgcgctggcccgttccggtgcggttgacgtcatcatcgtcgactccgtagcggcgttgacaccaaaagcagaaatcgaaggtgaaatcggtgactctcatatgggccttgcggcacgtatgatgagccaggcgatgcgtaagctggccggtaacctgaagaactccggtacgctgctgatctttatcaaccagatccgtatgaaaattggcgtgatgttcggtaacccggaaaccactaccggtggtaacgctctgaaattctacgcttctgtccgtctggatattcgccgcatcggcgcgatcaaagagggtgatgaagtggtgggtagcgaaacccgcgttaaagtggtgaaaaacaaaatcgcagcaccgtttaaacaggctgagttccagatcatgtacggcgaaggtatcaacgtttacggtgagctggtcgacctgggcgtgaagcacaagctgatcgaaaaagccggtgcctggtacagctataacggtgacaagattggtcagggtaaagccaactcaggtaacttcctgaaagagaacccggctatcgctaacgaaatcgaagcaaaactgcgtgaaatgctgttgaacagcccggacgataagcctgattttgttccggctccgcatgaagccgatagtgaagttaacgaagatatctaa

SEQ ID NO: 107 - DNA, 1842 bp, unidentified
  source 1..1842: mol_type = genomic DNA; note = RNA polymerase sigma factor RpoD sequence
atggagcaaaacccgcagtcacagcttaagctacttgtcacccgtggtaaggagcaaggctatctgacctatgccgaggtcaatgaccatctgccggaagatatcgtcgactccgatcagattgaagacatcattcagatgatcaacgacatgggcattcaggttgtagaagaagcgcctgatgccgatgatttgatgctgaatgagaacaacaacgacacggacgaagacgctgccgaagcggctgctcaggtattatccagcgtagaatctgaaatcggacgtaccaccgacccggtgcgcatgtacatgcgcgaaatggggacggttgaactgctgacgcgtgaaggcgagatcgatatcgccaaacgcatcgaagagggtatcaaccaggtacagtgttccgttgctgaatatcctgaagcgattacttacctgcttgagcaatatgaccgtgttgaagcgggcgaagcgcgcctgtcggatctgatcaccggttttgtcgacccgaatgccgaagcagagatcgcccctactgcgactcacgtgggttcagaactttccgctgaagagcgtgatgacgaagaagaagacgaagagtctgacgacgacagctcggatgatgacaacagcatcgatccggaactggcgcgggaaaaattcaacgacctgcgcgttcagtacgaaaccacccgtaccgttatcaaagcgaaaagccgcagccacgctgatgccatcgctgagatccagaatctgtccgacgtgttcaagcagttccgcctggtgccgaagcagttcgacttcctggtgaacagcatgcgcaccatgatggatcgcgtccgtactcaggaacgcctgatcctcaagctgtgcgtagaaatctgtaagatgccgaagaagaacttcattaccctgttcaccggtaatgaaaccagcgaaacctggttcaaagcggcactggcaatgaataagccgtggtcagagaagctgaacgatgtgtcagatgacgtacaccgtagcctgatgaagctgcagcagatcgaaacggaaactggcctgacgattgaacaggtaaaagacatcaaccgtcgtatgtcgatcggcgaagcgaaagcgcgccgtgcgaagaaagagatggttgaggctaacctgcgtctggttatctctatcgccaagaagtacaccaaccgtggcctgcagttcctggatctgattcaggaaggtaacatcggtctgatgaaagcggtggataagtttgaatatcgccgtggttataagttctcgacttatgccacctggtggatccgtcaggcgatcacccgttcaatcgctgaccaggcgcgtaccatccgtattccggtgcacatgattgagacgattaacaagctcaaccgtatttcccgccagatgctgcaagagatgggccgtgagccgacgccggaagagctggccgagcgtatgctgatgccggaagataagatccgtaaggtgctgaaaattgccaaagagccgatctctatggagacgccgattggtgatgatgaagattcacatctgggtgattttatcgaagacaccacgctggagctgccgctggactccgcgacgtcagagagcctgcgttctgccacgcacgacgtgctggccggtctgaccgcgcgtgaagccaaagtactgcgtatgcgtttcggtatcgatatgaataccgaccacacgctggaagaagtgggcaaacagttcgacgtaacgcgtgagcgtattcgtcagattgaggcgaaagcgctgcgtaagctgcgtcacccaagccgctctgaagtgctgcgcagcttcctcgacgattaa

SEQ ID NO: 108 - DNA, 4029 bp, unidentified
  source 1..4029: mol_type = genomic DNA; note = DNA-directed RNA polymerase subunit beta sequence
atggtttactcctataccgagaaaaaacgtattcgtaaggattttggaaagcgtccacaagttctggacattccatatctcctttctatccagcttgactcgttccagaagttcatcgagcaagatccggaaggtcaatatggtctggaagcagcattccgctccgtatttccaatccaaagctatagcggtaattctgagctgcagtacgtcagctaccgtttaggcgaacccgtctttgatgtgaaagagtgtcagattcgtggcgtcacgtattctgctcctctgcgcgtaaaactgcgcctggtgatctacgagcgcgaagcgccggaaggcaccgttaaagacatcaaagaacaagaagtttacatgggcgaaattccgctcatgacggataacggtacctttgttatcaacggtactgagcgcgttatcgtttctcagctccaccgtagtcctggtgtcttcttcgacagcgataagggtaaaacccactcgtccggtaaagtgctgtataacgcacgtatcatcccttaccgtggttcatggctggacttcgagttcgacccgaaagacaacctgttcgtccgtattgaccgtcgccgtaaactgccagcgaccatcattctgcgcgcgttgaattacaccactgaacagatcctcgacctgttcttcgataaagtggtttaccaaattcgcgacaacaagctgcagatggagcttattcctgagcgcctgcgtggtgagaccgcttcatttgatattgaagcgaacggcaccgtttacgtcgaaaaaggccgccgtattactgcgcgccatattcgccagcttgagaaagatgctgttgcccacatcgaagtgccggttgagtatattgccggtaaagtggtcgctaaagactacgttgatgagagcaccggtgaactgctgatcgcagcgaacatggaactgtcactggatctgctggctaaactcagccagtccggtcacaagcgcattgaaaccctgttcaccaacgatctggatcacggtgcgtacatgtctgagacggtacgtgtcgacccaaccagcgatcgcctgagcgctctggttgagatctaccgcatgatgcgtcctggtgagccaccaacgcgtgaagcggctgaaaacctgtttgagaacctgttcttctctgaagaccgctatgatctgtctgcggttggtcgtatgaagttcaaccgttctctgctgcgcgacgagatcgaaggttccggtatcctgagcaaagacgacatcattcaggtgatgaagaagctcatcggtatccgtaacggtattggcgaagtggatgatatcgaccacctcggcaaccgtcgtatccgttccgttggcgaaatggctgaaaaccagttccgtgttggccttgtgcgcgtagagcgtgcggtgaaagagcgtctgtccctgggcgatctggataccctgatgccacaggacatgatcaacgccaagccaatttctgcggcagtgaaagagttcttcggctccagccagctgtcacagtttatggaccagaacaacccgttgtctgagatcacgcataagcgtcgtatctctgcactgggtccgggcggtctgacgcgtgagcgtgcaggcttcgaagttcgagacgtacacccgacgcactacggtcgcgtatgtccaatcgaaacgccggaaggtccaaacatcggtctgatcaactccttgtctgtgtatgcacagaccaatgagtacggtttcctggaaaccccataccgtcgcgttcgcgaaggcgtggtgaccgacgaaattcattacctctctgctattgaagagggtaactacgttatcgctcaggcaaacaccaatctcgacgacgaaggtcacttcgtagacgacctggtcacctgccgtagcaaaggcgaatcgagtctcttcaaccgcgatcaagttgactacatggacgtttccacccagcaggtggtttccgtcggtgcgtcactgatcccgttcctggagcacgatgacgccaaccgcgcattgatgggtgcaaacatgcaacgtcaggcggttcctactctgcgtgctgataagccgctggtaggtaccggtatggagcgtgcggttgcggttgactccggtgttactgccgtagcgaaacgtggtggtaccgtgcagtacgtggatgcatcccgtatcgttattaaagttaacgaagacgaaatgtatccgggcgaagccggtatcgacatttacaacctgaccaaatatacccgttctaaccagaacacctgcatcaaccagatgccttgcgtgaacctgggtgagccaatcgaacgtggtgatgtgctggctgatggcccttcaaccgatctcggcgaactggcactcggtcagaacatgcgcgtcgcgttcatgccgtggaacggctacaacttcgaagactccattctggtctcggagcgcgttgttcaggaagatcgcttcaccactatccacattcaggaactggcgtgtgtgtctcgtgacaccaagctggggccagaagagatcaccgctgacatccctaacgtgggtgaagctgcgctctctaaactggatgagtccggtatcgtgtatatcggtgcggaagtgaccggtggggacattctggttggtaaggtaacacctaaaggtgaaacccagctgacgccagaagagaaactgctgcgtgcgatcttcggtgaaaaagcgtctgacgttaaagactcttctctgcgcgtaccaaacggtgtgtcagggacaatcatcgacgttcaggtctttacccgcgatggcgtggaaaaagacaagcgtgcgctggaaatcgaagagatgcagctgaagcaggcgaagaaagacctgtctgaagaattgcagatcctcgaagccggcttgttcagccgtattaactacctgctggttgccggcggtgttgaagcggaaaaactggagaagctgccacgtgagcgctggctcgaactgggcctgaccgacgaagagaagcaaaatcagctggaacagctggccgagcagtacgacgagctgaagcacgagtttgagaaaaaacttgaagccaagcgccgtaaaatcactcagggcgatgacctggcacctggcgtgctgaaaatcgtgaaagtgtatctggccgttaaacgtcagatccagcctggtgacaaaatggcaggtcgtcacgggaacaaaggtgttatctccaagatcaacccgatcgaagatatgccatacgatgagttcggtacgccggtcgacatcgtactgaacccgctgggcgttccatcacgtatgaacattggtcagattcttgaaacccacctgggtatggctgcgaaaggcattggcgagaaaattaacgctatgcttaagaagcaggaagaagtgtccaagctgcgtgaattcattcagcgtgcttacgatctgggcagcgatctgcgtcagaaagttgacctgaacaccttcaccgatgacgaagtgctgcgcctggcagagaatctgaaaaaaggtatgccaattgcaacaccagtgtttgacggcgcgaaagagagcgaaatcaaagagctgttacagctcggcggcctgccttcttctggccagatcacgctgtttgatggtcgtaccggtgagcagttcgaacgtcaggttaccgttggctacatgtacatgctgaagctgaaccacctggttgatgacaaaatgcatgcgcgttctaccggttcttacagcctcgttactcagcagccgctgggtggtaaggcgcagttcggtggtcagcgcttcggtgagatggaagtgtgggcactggaagcatacggtgccgcgtataccctgcaggaaatgctgaccgtgaagtctgatgacgttaacggccgtaccaagatgtataaaaacatcgttgacggcaaccatcagatggaaccgggcatgccggaatctttcaacgtactgttgaaagagatccgctcgctgggtatcaacatcgagctggaagacgagtaa

SEQ ID NO: 109 - DNA, 1700 bp, unidentified
  source 1..1700: mol_type = genomic DNA; note = DP68 Glutamine--tRNA ligase sequence
atgagcaagcccactgtcgaccctacctcgaattccaaggccggacctgccgtcccggtcaatttcctgcgcccgatcatccaggcggacctggattcgggcaagcacacgcagatcgtcacccgcttcccgccagagcccaacggctacctgcacatcggtcacgccaagtcgatctgtgtgaacttcggcctggcccaggagttcggtggcgtcacgcacctgcgtttcgacgacaccaacccggccaaggaagaccaggaatacatcgacgccatcgaaagcgacatcaagtggctgggcttcgaatggtccggtgaagtgcgctatgcgtccaagtatttcgaccagttgttcgactgggccgtcgagctgatcaaggccggcaaggcctacgtcgacgacctgaccccggagcaggccaaggaataccgtggcacgctgaccgagccgggcaagaacagcccgttccgtgaccgttcggtagaagagaacctcgactggttcaaccgcatgcgcgccggtgagttcccggacggcgcccgcgtgctgcgcgccaagatcgacatggcctcgccgaacatgaacctgcgcgacccgatcatgtaccgcatccgccacgcccatcaccaccagaccggtgacaagtggtgcatctacccgaactatgacttcacccacggtcagtcggacgccatcgaaggcatcacccactccatctgcaccctggagttcgaaagccatcgcccgctgtatgagtggttcctcgacagcctgccggttccggcgcacccgcgtcagtacgagttcagccgcctgaacctgaactacaccatcaccagcaagcgcaagctcaagcagttggtggacgaaaagcacgtgcatggctgggatgacccgcgcatgtccaccctgtcgggtttccgccgtcgcggctacaccccggcgtcgatccgcagcttctgcgacatggtcggcaccaaccgctccgacggcgtggtcgattacggcatgctcgagttcagcatccgtcaggacctggacgccaacgcgccgcgtgccatgtgcgtattgcgcccgttgaaagtcgtgatcaccaactatccggaagacaaggtcgaccacctcgaactgccgcgtcacccgcagaaagaagaacttggcgtgcgcaagctgccgttcgcgcgtgaaatctacatcgaccgtgatgacttcatggaagagccgccgaaaggctacaagcgcctggagcctaacggcgaagtgcgcctgcgcggcagctacgtgatccgtgccgatgaagcgatcaaggacgccgatggcaacatcgtcgaactgcgatgctcctacgacccggaaaccctgggcaagaaccctgaaggccgcaaggtcaaaggcgtcgttcactgggtgccggctgctgccagcatcgagtgcgaagtgcgcctgtacgatcgtctgttccgttcgccgaaccctgagaaggctgaagacagcgccagcttcctggacaacatcaaccctgactccctgcaagttctcacgggttgtcgtgccgagccatcgcttggcgacgcacagccggaagaccgtttccagttcgagcgcgaaggttacttctgcgcggatatcaaggactccaaacctggtcatccggtcttcaaccgtaccgtgaccttgcgtgattcgtggggccagtg

SEQ ID NO: 110 - DNA, 2418 bp, unidentified
  source 1..2418: mol_type = genomic DNA; note = DP68 DNA gyrase subunit B sequence
atgagcgaagaaaacacgtacgactcgaccagcattaaagtgctgaaaggtttggatgccgtacgcaaacgtcccggtatgtacatcggcgacaccgatgatggtagcggtctgcaccacatggtgttcgaggtggtcgacaactccatcgacgaagctttggccggtcactgcgacgacatcagcattatcatccacccggatgagtccatcaccgtgcgcgacaacggtcgcggtattccggtcgatgtgcacaaagaagaaggcgtatcggcggcagaggtcatcatgaccgtgcttcacgccggcggtaagttcgacgacaactcctataaagtttccggcggtttgcacggtgtaggtgtgtcggtggtgaacgctctgtccgaagagcttatcctgactgttcgccgtagcggcaagatctgggaacagacctacgtgcatggtgttccacaagaaccgatgaaaatcgttggcgacagtgaatccaccggtacgcagatccacttcaagccttcggcagaaaccttcaagaatatccacttcagttgggacatcctggccaagcgtattcgtgaactgtcgttccttaactccggtgtgggtatcgtcctcaaggacgagcgcagcggcaaggaagagttgttcaagtacgaaggcggcttgcgtgcgttcgttgagtacctgaacaccaacaagactgcggtcaaccaggtgttccacttcaacatccagcgtgaagacggtatcggcgttgaaatcgccctgcagtggaacgacagcttcaacgagaacctgttgtgcttcaccaacaacattccacagcgcgacggcggtactcacttggtgggtttccgttccgcactgacgcgtaacctgaacacctacatcgaagcggaaggcttggccaagaagcacaaagtggccactaccggtgacgatgcgcgtgaaggcctgacggcgattatctcggtgaaagtgccggatccaaagttcagctcccagaccaaagacaagctggtgtcttccgaagtgaagaccgcagtggaacaggagatgggcaagtacttctccgacttcctgctggaaaacccgaacgaagccaagttggttgtcggcaagatgatcgacgcggcgcgtgcccgtgaagcggcgcgtaaagcccgtgagatgacccgccgtaaaggcgcgttggatatcgccggcctgccgggcaaactggctgactgccaggagaaggaccctgccctctccgaactgtacctggtggaaggtgactctgctggcggttccgccaagcagggtcgtaaccgtcgcacccaggctatcctgccgttgaagggtaagatcctcaacgtcgagaaggcccgcttcgacaagatgatttcctctcaggaagtcggcaccttgatcacggcgttgggctgcggtattggccgcgatgagtacaacatcgacaaactgcgttaccacaacatcatcatcatgaccgatgctgacgtcgacggttcgcacatccgtaccctgctgctgaccttcttcttccgtcagttgccggagctgatcgagcgtggctacatctacatcgctcagccgccgttgtacaaagtgaaaaagggcaagcaagagcagtacatcaaagacgacgacgccatggaagagtacatgacgcagtcggccctggaagatgccagcctgcacttgaacgacgaagccccgggcatttccggtgaggcgctggagcgtttggttaacgacttccgcatggtaatgaagaccctcaagcgtctgtcgcgcctgtaccctcaggagctgaccgagcacttcatctacctgccttccgtgagcctggagcagttgggcgatcacgcccacatgcagaattggctggctcagtacgaagtacgtctgcgcaccgtcgagaagtctggcctggtttacaaagccagcttgcgtgaagaccgtgaacgtaacgtgtggctgccggaggttgaactgatctcccacggcctgtcgaactacgtcaccttcaaccgcgacttcttcggcagcaacgactacaagaccgtggttaccctcggcgcgcaattgagcaccctgttggacgacggtgcttacatccagcgtggcgagcgtaagaaagcggtcaaggagttcaaggaagccctggactggttgatggctgaaagcaccaagcgccacaccatccagcgatacaaaggtctgggcgagatgaacccggatcaactgtgggaaaccaccatggatcctgctcagcgtcgcatgctacgcgtgaccatcgaagacgccattggcgcagaccagatcttcaacaccctgatgggtgatgcggtcgagcctcgccgtgacttcatcgagagcaacgccttggcggtgtctaacctggatttctga

SEQ ID NO: 111 - DNA, 2832 bp, unidentified
  source 1..2832: mol_type = genomic DNA; note = DP68 Isoleucine--tRNA ligase sequence
atgaccgactataaagccacgctaaaccttccggacaccgccttcccaatgaaggccggcctgccacagcgcgaaccgcagatcctgcagcgctgggacagtattggcctgtacggaaagttgcgcgaaattggcaaggatcgtccgaagttcgtcctgcacgacggccctccttatgccaacggcacgattcacatcggtcatgcgctgaacaaaattctcaaggacatgatcctgcgttcgaaaaccctgtcgggcttcgacgcgccttatgttccgggctgggactgccacggcctgccgatcgaacacaaagtcgaagtgacctacggcaagaacctgggcgcggataaaacccgcgaactgtgccgtgcctacgccaccgagcagatcgaagggcagaagtccgaattcatccgcctgggcgtgctgggcgagtgggacaacccgtacaagaccatgaacttcaagaacgaggccggtgaaatccgtgccttggctgaaatcgtcaaaggcggtttcgtgttcaagggcctcaagcccgtgaactggtgcttcgactgcggttcggccctggctgaagcggaagtcgagtacgaagacaagaagtcctcgaccatcgacgtggccttcccgatcgccgacgacgacaagctggctcaagcctttggcctgtccagcctgccaaagcctgcagccatcgtgatctggaccaccaccccgtggaccatcccggccaaccaggcgctgaacgtgcacccggaattcacctacgccctggtggacgtcggtgatcgcctgctggtgctggctgaagaaatggtcgaggcctgcctggcgcgctacgagctgcaaggttcggtcatcgccaccaccaccggcactgcgctggagctgatcaatttccgtcacccgttctatgaccgtctgtcgccggtgtacctggctgactacgtagagctgggttcgggtactggtgtggttcactccgcgccggcctacggcgttgatgactttgtgacctgcaaagcctacggcatggtcaacgatgacatcctcaacccggtgcagagcaatggcgtgtacgcgccgtcgctggagttctttggcggccagttcatcttcaaggccaacgagccgatcatcgacaaactgcgtgaagtcggttcgctgctgcacaccgaaaccatcaagcacagctacatgcactgctggcgtcacaagaccccgctgatctaccgcgctaccgcgcagtggtttatcggcatggacaaagagccgaccagcggcgacaccctgcgtgtgcgctcgctcaaagcgatcgaagagaccaagtttgtcccggcctggggccaggcgcgcctgcactcgatgatcgccaaccgcccggactggtgcatctcccgccagcgcaactggggcgtgccgattccgttcttcctgaacaaggaaagcggcgagctgcacccacgtaccgttgaactgatggaagcagtggcgctgcgcgttgagcaggaaggcatcgaagcctggttcaagctggacgccgccgaactgctgggcgacgaagcgccgctgtacgacaagatcagcgacaccctcgacgtgtggttcgactcgggtaccacccactggcacgtgctgcgcggttcgcacccgatgggtcacgccaccggcccgcgtgccgacctgtacctggaaggctcggaccaacaccgtggctggttccactcgtcgttgctgaccggctgcgccatcgacaaccacgcgccgtaccgcgaactgctgacccacggcttcaccgtcgacgagacgggccgcaagatgtccaagtcgctgaaaaacgtgatcgagccgaaaaagatcaacgacaccctgggcgccgatatcatgcgtctgtgggtcgcctcgaccgattactcgggcgaaatcgccgtgtcggaccagatcctggcccgtagcgccgatgcctaccgccgtatccgtaataccgcacgcttcctgctgtcgaacctgaccggtttcaacccggccaccgacatcctgccggccgaggacatgctcgccctggaccgttgggccgtggaccgtacgctgttgctgcagcgcgagttgcaggaacactacggcgaataccgtttctggaacgtgtactccaagatccacaacttctgcgtgcaggagctgggtggtttctacctcgatatcatcaaggaccgccagtacaccaccggcgccaacagcaaggcgcgccgctcggcgcagaccgcgctgtaccacatctctgaagcgctggtgcgctggatcgcaccgatcctggccttcaccgctgacgaactgtgggaatacctgccgggcgagcgtaacgaatcggtgatgctcaacacctggtacgaaggcctgaccgaattgccggccaacttcgaactgggccgcgagtactgggaaggcgtgatggccgtcaaggttgcggtgaacaaggagctggaagttcagcgcgcggccaaggccgtcggtggcaacctgcaagccgaagtcaccctgtttgccgaggaaggcctgaccgccgacctggccaagctgagcaacgaactgcgcttcgtactgatcacctcgaccgcgagcctggcaccgtttgcccaggcacctgcggacgcagtggccaccgaagtgccgggcctcaagctcaaagtggtcaagtcggcctttcctaagtgcgcccgttgctggcactgccgtgaagacgtcggcgtgaacccagagcatccggaaatctgcggtcgttgcgtcgacaacatcagcggtgctggcgaggttcgccactatgcctaa

SEQ ID NO: 112 - DNA, 1785 bp, unidentified
  source 1..1785: mol_type = genomic DNA; note = DP68 NADH-quinone oxidoreductase subunit C/D sequence
atgactacaggcagtgctctgtacatcccgccttacaaggcagacgaccaggatgtggttgtcgaactcaataaccgttttggccctgacgccttcaccgcccaggccacacgcaccggtatgccggtgctgtgggtggcgcgcgccaagctcgtcgaagtcctgagcttcctgcgcaacctgcccaagccgtacgtcatgctttatgacctgcatggcgtggacgagcgtctgcgcaccaagcgtcaaggtttgccgagcggtgccgatttcaccgtgttctaccacttgatgtcgctggaacgtaacagcgacgtgatgatcaaggtcgcgctgtccgaaagcgacttgagcatcccgaccgtcaccggtatctggccgaatgccagctggtacgagcgcgaagtttgggacatgttcggtatcgacttcccgggccacccgcacctgacgcgcatcatgatgccgccgacctgggaaggtcacccgctgcgcaaggactttcctgcccgcgcaaccgaattcgacccgttcagcctcaacctcgccaagcagcagcttgaagaagaagctgcacgcttccgtccggaagactggggcatgaaacgctccggcaccaacgaggactacatgttcctcaacctgggcccgaaccacccttcggctcacggtgccttccgtatcatcctgcaactggacggcgaagaaatcgtcgactgtgtgccggacatcggttaccaccaccgtggtgccgagaagatggccgagcgccagtcctggcacagcttcatcccgtacaccgaccgtatcgactacctcggcggcgtgatgaacaacctgccgtacgtgctgtcggtcgagaagctggccggtatcaaggtgccggaccgcgtcgacaccatccgcatcatgatggccgagttcttccgcatcaccagccacctgctgttcctgggtacctatatccaggacgttggcgccatgaccccggtgttcttcaccttcaccgaccgtcaacgcgcctacaaggtgatcgaagccatcaccggtttccgcctgcacccggcctggtatcgcatcggcggcgtggcgcacgacctgccgaacggctgggagcgcctggtcaaggaattcatcgactggatgcccaagcgtctggacgagtaccaaaaggctgcgctggacaacagcatcctcaagggtcgtaccatcggcgtcgcgcagtacaacaccaaagaagccctggaatggggcgtcactggtgccggcctgcgttcgaccggctgcgacttcgacctgcgtaaagcacggccgtactcgggctacgagaacttcgagttcgaagtgccgctggccgccaatggcgatgcctacgaccggtgcatcgtgcgcgttgaagaaatgcgccagagcctgaagatcatcgagcagtgcatgcgcaacatgccggctggcccgtacaaggcggatcatccgctgaccacaccgccgccgaaagagcgcacgctgcagcacatcgaaaccctgatcacgcacttcctgcaagtttcgtggggcccggtgatgccggccaacgaatccttccagatgatcgaagcgaccaagggtatcaacagttattacctgacgagcgatggcggcaccatgagctaccgcacccggattcgtaccccaagctttgcccacttgcagcagatcccttcggtgatcaaaggcgagatggtcgcggacttgattgcgtacctgggtagtatcgatttcgttatggccgacgtggaccgctaa

SEQ ID NO: 113 - DNA, 1059 bp, unidentified
  source 1..1059: mol_type = genomic DNA; note = DP68 Protein RecA sequence
atggacgacaacaagaagaaagccttggctgcggccctgggtcagatcgaacgtcaattcggcaagggtgccgtaatgcgtatgggcgatcacgaccgtcaggcgatcccggctatttccactggctctctgggtctggacatcgcactcggcattggcggcctgccaaaaggccgtatcgttgaaatctacggtcctgaatcttccggtaaaaccaccctgaccctgtcggtgattgcccaggcgcaaaaaatgggcgccacctgtgcgttcgtcgacgccgagcacgccctggacccggaatacgccggtaagctgggcgtcaacgttgacgacctgctggtttcccagccggacaccggtgagcaagccctggaaatcaccgacatgctggtgcgctccaacgccatcgacgtgatcgtggtcgactccgtggctgccctggtaccgaaagctgaaatcgaaggcgaaatgggcgacatgcacgtgggcctgcaagcccgcctgatgtcccaggcgctgcgtaaaattaccggtaacatcaagaacgccaactgcctggtgatcttcatcaaccagatccgtatgaagatcggcgtaatgttcggcagcccggaaaccactaccggtggtaacgcgctgaagttctacgcttcggtccgtctggacatccgccgtaccggcgcggtgaaggaaggtgacgaagttgttggtagcgaaactcgcgttaaagtcgtgaagaacaaggtcgctccgcctttccgtcaggcagagttccagattctctacggcaagggtatctacctgaacggcgagatgattgacctgggcgtactgcacggtttcgtcgagaagtccggtgcctggtatgcctacaacggcagcaagatcggtcagggcaaggccaactcggccaagttcctggcagacaacccggatatcgctgccacgcttgagaagcagattcgcgacaagctgctgaccccagcgccagacgtgaaagctgccgccaaccgcgagccggttgaagaagtggaagaagctgacactgatatctga

SEQ ID NO: 114 - DNA, 1851 bp, unidentified
  source 1..1851: mol_type = genomic DNA; note = DP68 RNA polymerase sigma factor RpoD sequence
atgtccggaaaagcgcaacaacagtctcgtattaaagagttgatcacccttggtcgtgagcagaaatatctgacttacgcagaggtcaacgatcacctgcctgaggatatttcagatcctgagcaggtggaagacatcatccgcatgattaatgacatggggatccccgtacacgagagtgctccggatgcggacgcccttatgttggccgactccgataccgacgaggcagctgctgaagaagcggctgctgcgctggcagcggtggagaccgacatcggtcgtacgactgaccctgtgcgcatgtatatgcgtgaaatgggtaccgtcgagctgctgacacgtgaaggcgaaatcgaaatcgccaaacgtattgaagagggtatccgtgaagtgatgggcgcaatcgcgcacttccctggcacggttgaccacattctctccgagtacactcgcgtcaccaccgaaggtggccgcctgtctgacgttctgagcggctacatcgacccggacgacggcattgcgccgcctgccgccgaagtaccgccgcccgtcgatgcgaaagccgcgaaggctgacgacgacaccgaagacgacgatgctgaagccagcagcgacgacgaagatgaagttgaaagcggcccggacccgatcatcgcagcccagcgtttcggtgcggtttccgatcaaatggaaatcacccgcaaggccctgaaaaagcacggtcgctccaacaagctggcgattgccgagctggtggccctggctgagctgttcatgccgatcaagctggtaccgaagcaattcgaaggcttggttgagcgtgttcgcagtgcccttgaacgtctgcgtgcgcaagaacgcgcaatcatgcagctgtgtgtacgtgatgcacgtatgccgcgggctgacttcctgcgccagttcccgggcaacgaagtagacgaaagctggaccgacgcactggccaaaggcaaggcgaaatacgccgaagccattggtcgcctgcagccggacatcatccgttgccagcagaagctgaccgcgcttgagaccgaaaccggtctgacgattgctgaaatcaaagacatcaaccgtcgcatgtcgatcggtgaggccaaggcccgccgcgcgaagaaagagatggttgaagcgaacttgcgtctggtgatctcgatcgccaagaagtacaccaaccgtggtctgcaattcctcgatctgatccaggaaggcaacatcggcttgatgaaggcggtggacaagttcgaataccgtcgcggctacaagttctcgacttatgccacctggtggatccgtcaggcgatcactcgctcgatcgccgaccaggctcgcaccatccgtattccggtgcacatgatcgagacgatcaacaagctcaaccgtatttcccggcagatgttgcaggaaatgggtcgcgaaccgaccccggaagagctgggcgaacgcatggaaatgcctgaggataaaatccgcaaggtattgaagatcgctaaagagccgatctccatggaaacgccgattggtgatgacgaagactcccacctgggtgacttcatcgaagactcgaccatgcagtcgccaatcgatgtcgccactgttgagagccttaaagaagcgactcgcgacgtactgtccggcctcactgcccgtgaagccaaggtactgcgcatgcgtttcggcatcgacatgaataccgaccacacccttgaggaagtcggtaagcagtttgacgtgacccgcgagcggatccgtcagatcgaagccaaggcgctgcgcaagttgcgccacccgacgcgaagcgagcatctgcgctccttcctcgacgagtga

SEQ ID NO: 115 - DNA, 4074 bp, unidentified
  source 1..4074: mol_type = genomic DNA; note = DP68 DNA-directed RNA polymerase subunit beta sequence
atggcttactcatatactgagaaaaaacgtatccgcaaggactttagcaagttgccggacgtcatggatgtcccgtaccttctggctatccagctggattcgtatcgtgaattcttgcaggcgggagcgaccaaagatcagttccgcgacgtgggcctgcatgcggccttcaaatccgttttcccgatcatcagctactccggcaatgctgcgctggagtacgtgggttatcgcctgggcgaaccggcatttgatgtcaaagaatgcgtgttgcgcggtgttacgtacgccgtacctttgcgggtaaaagtccgcctgatcattttcgacaaagaatcgtcgaacaaagcgatcaaggacatcaaagagcaagaagtctacatgggcgaaatcccactgatgactgaaaacggtaccttcgtaatcaacggtaccgagcgtgttattgtttcccagctgcaccgttccccgggcgtgttcttcgaccacgaccgcggcaagacgcacagctccggtaaactcctgtactccgcgcggatcattccgtaccgcggttcgtggttggacttcgagttcgacccgaaagactgcgtgttcgtgcgtatcgaccgtcgtcgcaagctgccggcctcggtactgctgcgcgcgctcggttacaccactgagcaggtgctggacgctttctacaccaccaacgtattcagcctgaaggatgaaaccctcagcctggagctgattgcttcgcgtctgcgtggtgaaattgccgttctggacattcaggacgaaaacggcaaagtgatcgttgaagcgggtcgtcgtattactgcgcgccacatcaaccagatcgaaaaagccggcatcaagtcgctggaagtgcctctggactacgtcctgggtcgcaccaccgccaaggttatcgttcacccggctacaggcgaaatcctggctgagtgcaacaccgagctgaacaccgaaatcctggcaaaaatcgccaaggcccaggttgttcgcatcgagaccctgtacaccaacgacatcgactgcggtccgttcatctccgacacactgaagatcgactccaccagcaaccaattggaagcgctggtcgagatctatcgcatgatgcgtcctggtgagccaccgaccaaagacgctgccgagaccctgttcaacaacctgttcttcagccctgagcgttatgacctgtctgcggtcggccggatgaagttcaaccgtcgtatcggtcgtaccgagatcgaaggttcgggcgtgctgtgcaaggaagatatcgtcgcggtactgaagactctggtcgacatccgtaacggtaaaggcatcgtcgatgacatcgaccacctgggtaaccgtcgtgttcgctgcgtaggcgaaatggccgaaaaccagttccgcgttggccttgtgcgtgttgaacgtgcggtcaaagagcgtctgtcgatggctgaaagcgaaggcctgatgccgcaagacctgatcaacgccaagccagtggctgcggcagtgaaagagttcttcggttccagccagctttcccagttcatggaccagaacaacccgctctccgagatcacccacaagcgccgtgtttctgcactgggcccgggcggtctgacccgtgagcgtgctggctttgaagttcgtgacgtacacccgacgcactacggtcgtgtttgcccgatcgaaacgccggaaggtccgaacatcggtctgatcaactccctggccgcttatgcgcgcaccaaccagtacggcttcctcgagagcccgtaccgcgtggtgaaagacgctctggtcaccgacgagatcgtattcctgtccgccatcgaagaagctgatcacgtgatcgctcaggcttcggccacgatgaacgacaagaaagtcctgatcgacgagctggtagctgttcgtcacttgaacgagttcaccgtcaaggcgccggaagacgtcaccttgatggacgtttcgccgaagcaggtagtttcggttgcagcgtcgctgatcccgttcctggaacacgatgacgccaaccgtgcgttgatgggttccaacatgcagcgtcaagctgtaccaaccctgcgcgctgacaagccgctggtaggtaccggcatggagcgtaacgtagcccgtgactccggcgtttgcgtcgtagcccgtcgtggcggcgtgatcgactccgttgatgccagccgtatcgtggttcgtgttgccgatgatgaagttgaaactggcgaagccggtgtcgacatctacaacctgaccaaatacacccgctcgaaccagaacacctgcatcaaccagcgtccgctggtgagcaagggtgaccgcgttcagcgtagcgacatcatggccgacggcccgtccactgacatgggtgaactggctctgggtcagaacatgcgcatcgcgttcatggcatggaacggcttcaacttcgaagactccatctgcctgtccgagcgtgttgttcaagaagaccgtttcaccacgatccacattcaggaactgacctgtgtggcacgtgataccaagcttgggccagaggaaatcactgcagacatcccgaacgtgggtgaagctgcactgaacaagctggacgaagccggtatcgtttacgtaggtgctgaagttggcgcaggcgacatcctggtaggtaaggtcactccgaaaggcgagacccaactgactccggaagagaagctgctgcgtgccatcttcggtgaaaaagccagcgacgttaaagacacctccctgcgtgtacctaccggtaccaagggtactgttatcgacgtacaggtcttcacccgtgacggcgttgagcgtgatgctcgtgcactgtccatcgagaagactcaactcgacgagatccgcaaggacctgaacgaagagttccgtatcgttgaaggcgcgaccttcgaacgtctgcgttccgctctggtaggccacaaggctgaaggcggcgcaggtctgaagaaaggtcaggacatcaccgacgaagtactcgacggtcttgagcacggccagtggttcaaactgcgcatggctgaagatgctctgaacgagcagctcgagaaggcccaggcctacatcgttgatcgccgtcgtctgctggacgacaagttcgaagacaagaagcgcaaactgcagcagggcgatgacctggctccaggcgtgctgaaaatcgtcaaggtttacctggcaatccgtcgccgcatccagccgggcgacaagatggccggtcgtcacggtaacaaaggtgtggtctccgtgatcatgccggttgaagacatgccgcacgatgccaatggcaccccggtcgacgtcgtcctcaacccgttgggcgtaccttcgcgtatgaacgttggtcagatcctcgaaacccacctgggcctcgcggccaaaggtctgggcgagaagatcaaccgtatgatcgaagagcagcgcaaggttgctgacctgcgtaagttcctgcacgagatctacaacgagatcggcggtcgcaacgaagagctggacaccttctccgaccaggaaatcctggacttggcgaagaacctgcgcggcggcgttccaatggctaccccggtgttcgacggtgccaaggaaagcgaaatcaaggccatgctgaaactggcagacctgccggaaagcggccagatgcagctgttcgacggccgtaccggcaacaagtttgagcgcccggttactgttggctacatgtacatgctgaagctgaaccacttggtagacgacaagatgcacgctcgttctaccggttcgtacagcctggttacccagcagccgctgggtggtaaggctcagttcggtggtcagcgtttcggggagatggaggtctgggcactggaagcatacggtgctgcatacactctgcaagaaatgctcacagtgaagtcggacgatgtgaacggtcggaccaagatgtacaaaaacatcgtggacggcgatcaccgtatggagccgggcatgcccgagtccttcaacgtgttgatcaaagaaattcgttccctcggcatcgatatcgatctggaaaccgaataa

SEQ ID NO: 116 - DNA, 1674 bp, unidentified
  source 1..1674: mol_type = genomic DNA; note = DP69 Glutamine--tRNA ligase sequence
gtgcgcgaggacctggccagcggaaagcaccaggcgatcaagacccgcttcccgccggagccgaacggctacctgcacatcggccacgccaagtcgatctgcctgaacttcggcatcgccggtgagttcagcggcgtctgcaacctgcgtttcgacgacaccaatccggccaaggaagacccggagtacgtggccgcgatccaggacgacgtgcgctggctgggctttgaatggaacgagctgcgccacgcctcggactacttccagacctattacctggccgccgagaagctgatcgaacagggcaaggcctacgtctgcgacctgtcggccgaggaagtgcgcgcctaccgcggcaccctgaccgagccgggccgcccgtcgccgtggcgtgaccgcagcgtcgaggagaacctcgacctgttccgccgcatgcgtgccggtgaattccccgatggcgcgcgcaccgtgcgcgccaagatcgacatggccagcggcaacatcaacctgcgtgatccggcgctgtaccgcatcaagcacgtcgagcaccagaacaccggcaacgcgtggccgatctacccgatgtacgacttcgcccatgcgctgggcgattcgatcgagggcatcacccactcgctgtgcacgctggaattcgaagaccaccgcccgctgtacgactggtgcgtggacaacgtcgacttcgcccacgatgacgcgctgacccagccgctggtcgacgccggcctgccgcgcgaagcggccaaaccgcgccagatcgagttctcgcgcctgaacatcaactacacggtgatgagcaagcgcaagctgatggcgctggtcaccgaacagctggtggacggctgggaagacccgcgcatgccgaccctgcagggcctgcgtcgccgtggctacaccccggcagcgatgcgcctgttcgccgagcgcgtgggcatcagcaagcagaattcgctgatcgatttcagcgtgctggaaggcgcgctgcgcgaagacctggacagcgccgcaccgcgccgcatggccgtggtcgacccggtcaagctggtgctgaccaacctggccgaaggccacgaagagcagctgaccttcagcaaccacccgaaggacgagagcttcggtacccgcgaagtgccgttcgcacgtgaagtgtggatcgaccgcgaggacttcgccgaagtgccgccgaagggctggaagcgcctggttcccggtggtgaagtgcgcctgcgcggcgccggcatcatccgctgcgacgacgtgatcaaggatgccgacggcaccatcaccgagctgcgcggctggctggatccggaatcgcgcccgggcatggaaggcgccaaccgcaaggtcaagggcaccatccactgggtcagcgcggtgcacggtgtgccggccgagatccgcctgtatgaccgcctgttctcggtgccgaacccggacgatgaatcggaaggcaagacctaccgcgactacctcaatccggactcgcgccgcaccgtcaccggctatgtcgagccggcggctgccagcgctgcgccggaacagtcgttccagttcgagcgcaccggctacttcgttgccgaccgccgcgaccacaccgaagccaagccggtgttcaaccgcagcgtgaccctgcgcgacacctggtcggcctga

SEQ ID NO: 117 - DNA, 2460 bp, unidentified
  source 1..2460: mol_type = genomic DNA; note = DP69 DNA gyrase subunit B sequence
atgaccgacgaacagaacaccccggcaaacaacggcaactacgacgccaacagcattacggccctggaaggcctggaggctgtccgcaagcgcccaggcatgtacatcggcgacgtccatgacggcaccggcctgcatcacatggtgttcgaggtcgtcgacaactcaatcgacgaagccctcgccggccatgccgaccacgtctcggtgacgatccatgccgatggctcggtaggcgtgtccgacaacggtcgcggcatcccgacgggcaagcacgagcagatgagcaagaagctcgaccgcgatgtgtctgcagccgaagtggtgatgacggtcctgcacgcaggcggcaagttcgacgacaacagctacaaggtttccggcggcctgcacggcgtgggcgtcagcgtggtcaacgcgctgtcgcagaagctggtcctggatatctaccagggtggcttccactaccagcaggagtacgccgacggcgcagcactgcatccgctgaagcagatcggccccagcaccaagcgcgggaccaccctgcgcttctggccctcggtaaaggctttccacgacaacgtggaattccactacgacatcctggcccggcgcctgcgcgaactgtccttcctcaattccggcgtcaagatcgtgctggtggacgagcgtggtgatggccgccgcgacgacttccattacgagggcggcatccgcagcttcgtggagcatctggcgcagttgaagacgccgttgcacccgaacgtgatctcggtgaccggcgaatccaatggcatcaccgtggaagtggcgctgcagtggaccgactcctaccaggagacgatgtactgcttcaccaacaacattccgcagaaggacggcggtacccacctggccggcttccgtggcgcattgacccgcgtgctcaacaactacatcgagcagaacggcatcgccaagcaggccaagatcaacctgaccggcgatgacatgcgcgaaggcatgatcgcggtgctgtcggtgaaggtgccggatcccagcttctccagccagaccaaggaaaagctggtcagctcggatgtgcgcccggccgtggaaagcgcgttcggccagcgcctggaagagttcctgcaggaaaacccgaacgaagccaaggccatcgccggcaagatcgtcgacgctgcccgtgcccgcgaagcggcgcgcaaggcccgcgacctgacccgccgcaagggtgcgctggatatcgccggcctgccgggcaagctggccgactgccaggaaaaggatccggcgctgtccgaactgttcatcgtcgagggtgactcggcaggtggttcggccaagcagggtcgcaaccgcaagaaccaggcggtgctgccgctgcgcggcaagatcctcaacgtggaacgtgcgcgcttcgaccgcatgctggcgtccgaccaggtgggtacgctgatcaccgcgctgggtaccggcatcggtcgtgacgagtacaacccggacaagctgcggtaccacaagatcatcatcatgaccgacgccgacgtcgacggcgcgcacatccgcaccctgctgctgacgttcttctaccgtcagatgccggagctgatcgagcgcggttatgtctatatcggcctgccgccgttgtacaagatcaagcagggcaagcaggagctgtacctgaaggacgacccggcgctggacagctatctggccagcagcgcggtggagaacgctgggctggtgccggccagcggcgagccgccgatcgacggcgtggcactggaaaagctgctgctcgcctacgctgccgcgcaggacacgatcaaccgcaatacccaccgctacgaccgcaacctgctcgaagcgctggtcgacttcatgccgctggagctggaaaacctgcgcactgcaggtcctggcgaaggtctggacgcgttggccaagcacctcaaccagggcaacctcggcagcgcccgcttcaccctggaactgcaggaacccaacgagcagcgtccggcggccgtactggtgacccgcagccacatgggcgaacagcacatccaggtgctgccgctgtccgcgctggaaagcggcgaactgcgcggcatccatcaggcagcgcagctgctgcacggtctggtccgcgaaggcgcggtcatcacccgtggcgccaagtcgatcgagatcgactcgttcgcacaggcccgcaactggctgttggacgaagccaagcgcggccggcagatccagcgattcaagggtctgggcgaaatgaatccggaacagctgtgggataccaccgtcaatcccgatacccgtcgcctgctgcaggtgcgcatcgaagacgcggtggccgctgaccagatcttcagcaccctgatgggtgatgtggtcgaaccgcgtcgtgacttcatcgaagacaacgcgttgaaggtcgccaacctggatatctga

SEQ ID NO: 118 - DNA, 2835 bp, unidentified
  source 1..2835: mol_type = genomic DNA; note = DP69 Isoleucine--tRNA ligase sequence
gtgagccaggactacaagaccaccctcaacctgccggccaccgaattcccgatgcgcggcgacctgcccaagcgcgagccgggcattctggcgcgctgggaagagcaggggctctaccagcagctgcgcgacaacgccgccggccgcccgctgttcgtgctgcatgacggcccgccgtacgccaatgcgcgcatccacctgggccatgcggtcaacaagatcctcaaggacatcatcgtcaagtcgcgctacctggccggcttcgatgcgccctacgtgccgggctgggactgccatggcctgccgatcgaaatcgcggtggaaaagaagtggggcaaggtcggggtgaagctcgatgcggtcgagttccggcagaagtgccgcgagttcgccgaagaacagatcgacatccagcgtgccgacttcaagcgcctgggcgtcaccggcgactgggacaacccgtacaagaccctaagcttcgatttcgaggccaacgagatccgtgcgctgtccaagatcgtggccaacggccatctgctgcgtggcgccaagccggtctactggtgcttcgactgcggctcggcactggccgaggccgagatcgagtaccacgagaagacctcgccggcgatcgacgtggcctacaccgcgcgtgatccgcaggcggtggcgcaggcgttcggcgtcagcctgccggccgatgtcgaagtggcggtgccgatctggaccaccactccgtggacgctgccggcttcgctggcggtgtcgctgggcgcggacatccgctacgtgctggccgaaggcccggcgcacaacggcaagcgccgttggctggtgctggctgctgcgctggccgaacggtcgctgcagcgctacggcgtggacgcggtggtgctgcacggtgaagccgaaggttcggcgctggaaaaccagctgctggcgcacccgttctacccggagcgcgagatccccgtgctcaacggcgaacacgtgtccgacgaggacggtaccggtgcggtgcacactgcccccggccacggccaggaagactacgtggtcagccagaagtacggcctgctggagaagtacaacgccggccagatcaatccggtcgacggtgcgggcgtgtacctggcgtccaccccgcccgccggtgacctggtgctggccggtacccacatctggaaggcgcagcagccgatcatcgaagtgctggccgccagcggcgcgctgctcaaggccgtggagatcgtgcacagttatccgcattgttggcgccacaagaagaccccgctggtgttccgcgccaccccgcagtggttcatttcgatggacaaggccaacctgcgcaacgatgcgctggccgcgatcgataccgtcggctggttcccgagctggggcaaggcgcgcatccaaagcatgatcgacggccgcccggactggaccatctcgcgccagcgcacctggggcgtgccgatcgcgctgttcacccaccgccagaccggcgagatccacccgcgttcggtggagctgatgcagcaggtggccgaccgcgttgaagccgaaggcatcgacgtgtggtactcgctggatgcggctgaactgctgggcgctgaagcggccgactacgagaaggtcaccgacatcctcgatgtctggttcgattccggcgtgacccacgaagccgtgctggctgcccgtggcttcggcaagccggccgatctgtacctggaaggttcggaccagcatcgcggctggttccagtcctcgctgctgaccggcgtggccatcgacaagcgcgcgccgtacaagcagtgcctcacccacggtttcaccgtggacgagcacggccgcaagatgtccaagtcgctgggcaacggcatcgaaccgcaggaaatcatgaacaagctgggcgcggacatcctgcgcctgtggatcgcctcggccgactacagcaacgagatgtcgctgtcgcaggaaatcctcaagcgcaccgccgacgcctaccgccgcctgcgcaacaccgcccgcttcctgctgggcaacctggacggtttcgatccggcccagcacctgcgcccgctcaacgagatggtcgcgctggaccgctggatcgtgcatcgcgcctgggagctgcaggagaagatcaaggcggcgtatgacaactacgacatggccgagatcgtgcagttgctgctgaacttctgcagcgtggacctgggctcgctgtacctggacgtgaccaaggatcgcctgtatacgatgccgaccgattcggatggtcgtcgttcggcgcagagcgcgatgtaccacatcgccgaagcgttcacccgctgggtggcgccgatcctgaccttcaccgccgacgagctgtggggctacctgccgggcgatcgtgccggccacgtgctgttcactacctggtacgagggcctggcaccgctgccgaccgatgcacagctcaacgctgccgacttcgatcagctgctggccgtgcgcgagcaggtggccaaggtgctggagccgatgcgcgccaatggtgcgatcggtgccgcgctggaagcggagatcaccatcgccgccagcgaagagcaggccgcgcgctggcagccgctggccgatgaactgcgtttcctgttcatcagtggtgacgtgcaggtgcgtccggcgaccaccgacgaggtgttcgtcagcgcgcagccgacgcagaagtccaagtgcgtgcgctgctggcaccaccgtgccgacgttggcagcaatgccgaccacccggaactgtgcggccgctgcgtgaccaacatcgccggtgccggcgaagcgcggagctggttctga

SEQ ID NO: 119 - DNA, 2076 bp, unidentified
  source 1..2076: mol_type = genomic DNA; note = DP69 Glycine--tRNA ligase beta subunit sequence
atgagccacttgtctcccctgctgattgaactgggcaccgaagagttgccggtcaaggcgctgccgggcctggcccaggccttcttcgacggtgttgtcgatggcctgcgcaagcgcggcgtcgaactggagctgggcgatgcccgcccgctgtcgaccccgcgccgcctggccgtgctgctgccgggcgttggcctggaacagccggaacaacacagcgaagtgctgggcccgtacctgaacatcgcgctggacgccgaaggccagccgaccaaggcgctgcagggtttcgcggccaaggccgggatcgactggaccgcgctggagaagaccaccgacaacaagggtgagcgcttcgtgcaccgtgcggtgactccgggcgcgcgcaccgctgcgctgctgccggagatcctgcgcgaggccatcgccggcatgccgattcccaagccgatgcgctggggcgaccacagctggggcttcgcccgcccggtgcactggctggtgctgctgcatggcggcgacgtggtcgaggccgaactgtttggcctgaaggccgaccgcatgagccgcggccaccgcttcctgcacgacaagaccgtgtggctgacccagccgcaggactatgtcgaatcgctgcgcgccgccttcgtgctggtcgatccggccgagcgccgccggcgcatcgttgccgaagtggaagccgctgccgccaccgccggtggcagcgcacgcatcaccgaggacaacctggagcaggtggtgaacctggtcgagtggccggcggcagtgttgtgcagcttcgagcgcgcgttcctggcggtaccgcaggaagcgctgatcgagacgatggagatcaaccagaagttcttcccggtgctggatgacggcggcaagctgaccgagaagttcatcggcatcgccaacatcgagtccaaggacgtggccgaagtggccaagggctacgagcgcgtgatccgcccgcgcttcgccgatgccaagttcttcttcgacgaagacctgaagcagggcctgcaggcgatgggcgagggcctgaagacggtgacctaccaggccaagctgggcagcgtggccgacaaggtcgcgcgcgtggcggcgctggccgaggtgatcgctgcgcaggtgggggccgacccggtgctggccaagcgtgccgcgcagctggccaagaacgacctgcagtcgcgcatggtcaatgagttcccggaactgcagggcatcgctggccgccactacgcggtggccggtggcgagtcgccggaggtggcgctggccatcgacgaggcctaccagccgcgcttcggtggcgatgacatcgcgctgtcgccgctgggcaaggtgctggcgatcgccgagcgtgtggacacgctggccggcggtttcgccgcgggcctgaagccgaccggcaacaaggacccgttcgccctgcgccgcaacgcgctgggcctggcccgcacgattatcgaaagtggcttcgagctggacctgcgcgcgctgctggccagcgccaatgccgggctgaccgtgcgcaacgtgcaggccgacgtggctgagctgtacgacttcatcctcgaccgcctgaagggctactacagcgacaagggcgtgccggccagccacttcaatgcggtggctgagctgaagccggtctcgctgtacgatttcgaccgtcgcctggacgccatcggtatcttcgcggcgctgccggaggccgaggcgctggcagcggccaacaagcgcatccgcaacatcctgcgcaaggccgaaggcgatattccgggccagatcgatgcggccctgttgcaggaagatgccgagcgcgcgctggcggaagccgtgactgcagccatcgacgacaccggcgccagcctgcaccagaaggactacgtggccgtgctggcgcgcctggcccgcctgcgtccgcaggtcgatgcgttcttcgatggggtgatggtcaatgccgaggatccggcactgcgcggcaaccgcctggcgctgctgacgatgctgggcgagcgcttgggcaaggtcgcggcgatcgagcatctgtcgagctga

SEQ ID NO: 120 - DNA, 1410 bp, unidentified
  source 1..1410: mol_type = genomic DNA; note = DP69 Glutamine synthetase sequence
atgtccgtggaaaccgtagagaagctgatcaaggacaaccagatcgagttcgtcgatctgcgcttcgtcgacatgcgtggtgtcgaacagcatgtgaccttcccggtcagcatcgtcgagccgtcgctgtttgaagaaggcaagatgttcgatggcagctcgatcgccggctggaagggcatcaacgagtcggacatggtgctgctgccggacaccgccagcgcctacgtcgacccgttctacgccgatccgaccatcgtgatcagctgcgacatcctcgacccggccaccatgcagccgtatggccgttgcccgcgcggcatcgccaagcgcgccgagtcctacctgaagtcctcgggcatcgccgaaaccgcgttcttcggcccggagccggagttcttcatcttcgactcggtgcgtttcgccaatgaaatgggcaacaccttcttcaaggtcgactcggaagaagcggcgtggaacagcggcgccaagtacgacggcgccaacagcggctaccgtccgggcgtgaagggcggttatttccccgttccgccgaccgacaccctgcacgacctgcgtgcggagatgtgcaagaccctggaacaggtcggcatcgaagtggaagtgcagcaccacgaagtggccaccgccggccagtgcgagatcggcaccaagttcagcaccctggtgcagaaggccgacgaactgctgcggatgaagtacgtcatcaagaacgtcgcccaccgcaacggcaagaccgtcaccttcatgcccaagccgatcgtcggcgacaacggcagcggcatgcacgtgcaccagtcgctgtccaagggcggcaccaacctgttctccggtgacggctacggtggcctgagccagatggcgctgtggtacatcggcggcatcttcaagcatgccaaggcgatcaacgcctttgccaactcgggtaccaacagctacaagcgcctggtgccgggcttcgaagccccggtgatgctggcctactcggcgcgcaaccgttcggcctcgtgccgcattccgtgggtgtccaacccgaaggcgcgtcgcattgaaatgcgcttccccgatccgatccagtcgggctacctgaccttcaccgcgctgatgatggccggcctggacggcatcaagaaccagatcgacccgggcgcaccgagcgacaaggatctgtacgacctgccgccggaagaagagaagctgattccgcaggtctgctcctcgctggaccaggccctggaagcgctggacaaggaccgtgagttcctcaaggccggtggcgtgatgagcgatgacttcatcgacggctacatcgcgctgaagatgcaggaagtgaccaagttccgcgcggcgacccacccgctggaataccagttgtactacgccagctga

SEQ ID NO: 121 - DNA, 1515 bp, unidentified
  source 1..1515: mol_type = genomic DNA; note = DP69 Glucose-6-phosphate isomerase sequence
atgacaacgaacaacggattcgactcgctgcattcccacgcccagcgcctgaagggcgcaagcatccccagcctgctcgccgccgaacccggccgcgtacaggacctggcgctgcgggtcggtccgttgtatgtcaacttcgcccggcagaaatacgatgccgcggcgttgcaggcgctgttggcgctggctgccgaacgtgatgtcggcggcgccatcacgcgcctgttccgtggcgagcaggtcaatctgaccgaaggccgcgccgcactgcacaccgcactgcgcggcgacgtggtcgatgcgccggttgccgccgaggcctatgccacggcccgcgaaatccgccagcgcatgggcgtgctggtgcgcgcactggaagacagtggcgtgaccgatgtggtcagtgtcggcatcggcggttccgatctcggtccgcgtctggtcgccgacgcactgcgtccagtcactggcgctcgcctgcgcgtgcatttcgtgtctaacgtggacggcgctgccatgcagcgcacgctggccacgctggatccggcgaagaccgccggcatcctcatttccaagaccttcggtacccaggaaaccctgctcaacggccagatcctgcacgattggctgggtggcagcgagcgcctgtacgcggtcagcgccaatccggaacgcgccgccaaggccttcgccatcgccgccgagcgcgtgctgccgatgtgggactgggtaggggggcgctattcgctgtggtcggccgtcggtttcccgatcgcactggccatcggcttcgagcgtttcgagcagttgctggaaggcgccgcgcagatggatgcgcatgcgctggacgcgccgctggagcgcaacctgccggtgctgcacggcctgaccgacatctggaaccgcaatctgctgggctctgccacgcatgcggtgatgacctacgaccagcgcttggcgctgctgccggcctacctgcagcagctggtgatggaaagcctgggcaagcgcgtgcagcgcgatggccagccggtcaccaccgacaccgtgccggtgtggtggggcggtgccggcaccgatgtgcagcacagcttcttccaggccctgcaccagggcaccagcatcattccggccgatttcatcggctgcgtgcacaacgacgatccgtatacggtcaaccaccaggcgttgatggccaacctgctggcgcagaccgaagcgctggccaacggccagggcagtgacgatccgcaccgcgattatccgggtggccgcccgagcacgatgatcctgctcgacgcgctcaccccgcaggcgctgggcgccttgatcgcgatgtacgaacacgccgtgtacgtgcagtcggtgatctggaacatcaacgccttcgaccagttcggtgtcgagctgggcaagcagctggccagtggcctgctgcccgctctgcagggtgaggatgtcgaggtcaacgacccgctgacccgtgagctgctggcccagctgaagggctga

SEQ ID NO: 122 - DNA, 2640 bp, unidentified
  source 1..2640: mol_type = genomic DNA; note = DP69 Leucine--tRNA ligase sequence
atgaccagcgtcgaacccaacgtttacgatccgcagcaggttgaatccgccgcccagaagtactgggacgctacccgtgccttcgaggtcgatgaagcctcggacaagccgaagtactactgcctgtcgatgcttccgtatccgtccggtgcgctgcacatgggccacgtgcgcaattacacgatcggcgacgtgatcagccgctacaagcgcatgaccggccacaacgtgctgcagccgatgggctgggacgcgtttggcctgccggcggaaaacgctgcgatcaagaacaagaccgcgccggccgcctggacctacaagaacatcgaccacatgcgcagccagctgcagtcgctgggctatgccatcgactggtcgcgcgagttcgccacctgccgcccggactattacgtccacgagcagcgcatgttcacccgcctgatgcgcaagggcctggcctaccgccgcaacgcggtggtgaactgggacccggtcgaccagaccgtgctggccaacgagcaggtcatcgacggccgtggctggcgctccggcgcgcttgtggaaaagcgcgagatcccgcagtggttcctgcgcatcaccgactacgcccaggaactgctggacggcctggatgagctggacggctggccggagtcggtcaagaccatgcagcgcaactggatcggccgctccgaagggctggaaatccagttcgacgtgcgcgacgtcgatggtgccgcactggatccgctgcgcgtgttcaccacccgcccggacaccgtgatgggcgtgactttcgtgtcgatcgcggccgaacatccgctggcgctgcatgccgcgaagaacaacccggaactggctgcgctgctgtcggaaatgaagcagggcggcgtgtccgaggccgagctggagacccaggaaaagcgcggcatggataccggcctgcgcgccgtgcatccggttaccggtgcccaggtgccggtgtgggtcgccaacttcgtgctgatgggctacggcactggcgcggtgatggccgtaccgggccacgaccagcgcgacaatgaattcgccaacaagtacaacctgccgatccgccaggtcatcgcgctgaagtcgctgcgcaaggacgaaggcgcctacgacgcgacgcgctggcaggactggtacggcgacaagacccgcgagaccgaactggtcaactccgaagagttcgacggcctggacttccagggcgctttcgaggcgctggccgaacggttcgagcgcaaggcccagggacagcgccgggtgaactaccgcctgcgcgactggggcgtgagccgccagcgctactggggctgcccgattccggtgatctactgcgacaagtgtggcgcggtaccggtgccggaagaccagctgccggtggtgctgccggaagacgtggcgttcgccggtaccggttcgccgatcaagaccgatccggaatggcgcaagaccacctgcccggactgcggcggtgcggccgagcgtgagaccgacaccttcgacaccttcatggagtcgagctggtactacgcccgctacacctcgccgggcgcccgcgatgcggtcgacaagcgcggcaactactggctgccggtggaccagtacatcggtggcatcgaacacgcgatcctgcacctgatgtatttccgcttctaccacaagctgctgcgcgacgcgcggatggtggacagcaacgaacccgcgcggaacctgctgtgccagggcatggtgatcgctgagacctactaccgcccgaacccggacggctcgaaggactggatcaacccggccgatgtggaagtgcagcgcgacgagcgcggccgcatcaccggcgccaccctgatcgccgacggtcagccggtggtggtcggtggtaccgagaagatgtccaagtcgaagaacaacggcgtggacccgcaggcgatggtcggcaagtacggcgccgataccgtgcgcctgttctcgatgttcgctgcaccgccggaacagtcgctggaatggaacgaagccggcgtggacggcatggcccgcttcctgcgccgcctgtgggcacaggtgcagaagcacgctgccgagggtgccgcaccggcgctcgacgcggccgcgctggatgccggccagaaggccctgcgccgcaagacccacgagaccatcggcaaggtcggcgacgactacggccgccgccacagcttcaacaccgccattgccgcggtgatggagctgatgaacgcgctggccaagttcgaggacggcagtgaacaggggcgcgccgtgcgccaggaagcactgcaggccatcgtgctgctgctcaacccgatcaccccgcatgccagccacgccctgtggcaggtactgggccatggcgaaacgctgctggaagatcagccgttcccgcaggccgacagcagtgcgctggtgcgcgatgcgctgactttggccgtgcaggtcaatggcaagctgcgtggcaccatcgaggtcgccgccgatgccgcgcgcgagcagatcgaagcgctggccctggccgagccgaacgcggccaagttcctggaaggcctgacggtgcgcaagatcatcatcgttcccggcaagatcgtgaacatcgtcgctgcctga

SEQ ID NO: 123 - DNA, 2070 bp, unidentified
  source 1..2070: mol_type = genomic DNA; note = DP70 Glycine--tRNA ligase beta subunit sequence
atgtctaaacatacagtattgttcgaattgggctgtgaagaacttccacctaaaagcctcaaaaaattacgtgatgcactgcatgctgaaacggtaaaaggcttaaaagatgcaggcttagcattcgactcaatcgaagcttatgcagcaccgcgtcgtttggcacttaaaattgtgaatatcgatggcgctcagcctgatacacaaaaacgctttgacggccctgcaaaagaagcggcttatgatgctgaaggcaaaccaagcaaagcattagaaggctttatgcgtggtcaaggcatcactgcggatcaagtcaccacgttccaagcgggtaaagttgaaaaggtttgctatttaaaagatgttaaaggtcaaagccttgaggttttactgccacaaattctacaagcagctttggacaatcttccaattgcaaaacgtatgcgttcagcggcaagccgtactgaattcgtgcgtcctgtaaaatgggtggtgttgctcaaagacaatgatgtgattgcagccactattcaagatcacaaagcaggcaatgtgacttatggtcatcgtttccatgcccctgaagcgattactttggctcatgcagatgaatatcttgccaagttaaaagcggcttatgtggttgctgactttgcagaacgccaagccatcattgaccaacaagtcaaagcgttggctgatgaagttaatgcgattgcgattgtaccaagcgacctgcgtgatgaagtgaccgcattggtggaatggcctgttgcgctacgtgccagctttgaggagcgtttccttgctgtaccgcaagaagctttgattaccacgatgcaagacaaccaaaaatacttctgtttggtgaatagtgataacaagctacagccttatttcattactgtttcaaatattgagtctaaagatccgattcaaattattgaaggcaatgaaaaagtggttcgtccacgtttgtcggatgctgaattcttcttcttgcaagatcaaaagcaaccactagcttctcgtaaagaaaaactggctaacatggtgttccaagcacaattgggtacgctgtgggataagtcacaacgtattgcaaaattggctgtggctttatcgaacatcacgggtgcaactgcggctgatgctgaaaaagcagcattgctggcaaaatgtgacttaacctctgaattggtgggtgaattccctgaacttcaaggcattgcgggaacctattacgcacgcattgaaggtgaaaaccatgaagtggctgaagctttaggcgaacagtatttacctaaatttgcaggcgatgttttaccgcaaacaaaaacaggcacaaccattgcccttgccgaccgtttagacacgctcacgggtatttttggtattggtcaagcacctacaggttctaaagatccgtttgcattacgtcgttctgcaatcggtattttacgtttggtgactgaaaacaatcttgatgtgtcgattgaagatttaatccagctggcattaaacgcttatggcgatgttgtagcggatcatgcgaagactttagcggatgctgttgcattccttgaaggtcgttaccgtgccaagtatgaagaccaaggcgttgcagttgatgtgattcaagcggttcaagcattatcaccaaaatcacctttagattttgataagcgtgtgactgcggtaaatcatttccgtgcattgcctgaagctgctgcactggctgctgcaaataagcgtgttgccaacattcttgccaaagaagcagaactaacaggcgcagtggttgaagcaaacttggttgaagaggctgaaaaagcattattcgctgtacttgctaaaattacgcctgaagttgaaccattatttgctgccaaagattacaccactgcattgtctaagcttgctgctttacgtgcgcctgtggatgcattctttgaaggcgtcatggtcatggcagatgatgcagaattgaaagccaaccgtttacgtttattggctcaattacgtggtttgtttacaagtgttgcggatatttcggtgttgcagcactaa

SEQ ID NO: 124 - DNA, 2472 bp, unidentified
  source 1..2472: mol_type = genomic DNA; note = DP70 DNA gyrase subunit B sequence
atgagttcagaagatcaagctgcttctcaaacagaacaaaccaatgaaaaggcttatgattcctctagtatcaaagtattacgtggcctagatgctgttcgtaagcgtccgggtatgtatattggtgatacggacgatggttcaggtttacatcacatggtgtttgaggtggtcgataatgcgattgatgaagccttagcgggtcactgtgatgaaatcttagtcaccatccatgaagatgagtctgtaagtgttgcagataacggtcgtgggattccaacggatattcaccctgaagaaggggtatctgccgctgaagtgattttaaccattttgcatgctggcggtaagtttgatgataatagctataaagtttccggtggtttacacggggtaggtgtttctgttgtaaatgccttgtcgagtaaattattactaaatattcgtcgtgcaggaaaagtatatgaacaggaatatcaccatggtgatcctgtctatccattacgcgcgattggtgatactgaagaaaccggtaccaccgttcgtttctatccgagtgaattaaccttctctcaaacgatttttaatgttgatattttagcgcgtcgtttgcgcgaactttcattcttaaatgcaggggttcgtattgtattacgtgatgaacgtatcaatgctgaacatgtatttgattatgaaggtggtttgtctgaatttgtaaaatatatcaatcaaggtaaaacccacttgaatgagatttttcattttaccagtgaagttgtggaaacaggaattactgttgaagtagcattacagtggaatgatacttatcaagaaaatgtccgttgctttaccaataacatcccacaaaaagatggtggtacgcatttagccggtttccgtgccgcgttaacacggggtttaaaccagtatcttgatagtgaaaatattcttaagaaagaaaaagttgctgtcacaggtgatgatgcccgtgaaggtttaacggcgattgtttcagtgaaagtgcctgatccaaaattctcatcacaaaccaaagaaaaattggtttccagtgaagtgaaaactgctgtagagcaggcgatgaacaagtctttttctgaatatcttttagaaaatccacaagcggctaaatcgattgccggcaaaattattgatgctgcacgtgcacgtgatgctgcgcgtaaagcacgtgaaatgacacgtcgtaagagtgcattagatattgctggtctgcctggtaaactggcggattgccaagaaaaagatccagcattgtctgaactttacttggtcgaaggtgactcggcgggcggttctgcaaaacagggtcgtaaccgtaagatgcaagctattctgccgcttaaaggtaaaatcttaaacgtagaacgtgcacgttttgacaaaatgatttcatcgcaagaagtgggcacgctgattactgcactgggctgtggtattggtcgtgaggaatacaatcctgataaattgcgttatcacaaaatcattatcatgaccgatgccgacgtcgatggttcgcacattcgtacgctcctgttgaccttcttcttccgtcaaatgccagaacttgtggaacgtggttatatttatattgcacagccaccgttgtataagttgaaaaaaggtaagcaagagcaatatcttaaagataatgatgctttagaaacctatcttatttcgaatgccattgatgagcttgaactgcatattagtgctgaggcacctgcgattcgtggtgaatctttggctaaagtgattgctgattatcaaacctcacaaaaaagtttaaatcgtttaacgctacgttatcctgcaagcttgctggatggtttacttggtttggatgcatttaaacttgatcaaaatcatgatgaagattatgtaaaacaatggtctgaacaattgcgtgcagcaattgaacaacaccaaccaagtttgcgtcctgaaatcaccttagaagcttttgaaaaagagcatgcagatggtgagaaagtgacgcattattggccacgtgtaacggtctatgtacataacttgccgcatcattatttacttgattctggattattggcttcaagtgaatacaagcgtttactgcaaaattcgaagagttggttcacattgcttgaagatggcgcttatttgcaaaaaggtgagcgtaaaattcatgtcgccactttccatcaagtttggcaacatattttatccgactcgcgtcgtggcatgatgatccagcgctataaaggtttgggtgagatgaacgcggaacagctttgggaaaccaccatggatcctgaaaaccgtaacatgttgcaagtcaccattaatgatgcgattgaagcggatcgtatgttctcttgtttgatgggagatgatgtggaaccacgtcgtgccttcattgaagaaaatgctttaaatgcggatattgacgcttaa

SEQ ID NO: 125 - DNA, 2625 bp, unidentified
  source 1..2625: mol_type = genomic DNA; note = DP70 Leucine--tRNA ligase sequence
atgactacttctcacattgaccctgaatatcaagcgagcgcgattgaatccactgtccaacaagactgggaaactcgcaaagcctttaaagttgccgacactgtagaaggtaaacatcgttatatcctctcgatgttcccttatccaagtggcaagctgcatatgggtcatgtgcgtaactacaccattggcgacgtgattagccgtttccaccgtctcaaaggtgaaactgtcctacaaccgatgggttgggatgcttttggtctgcctgcggaaaatgcagcgattgcacaccaagttgcccctgcaaaatggacctttgaaaacatcgcgtacatgcgtgaccagttaaaaaaattgggtctgtcagtcgattgggatcgtgaatttgcgacctgtacgccagagtattatcactgggaacaatggttatttgtacagctgtataaaaaagggctgatttatcgcaaactttcaacggtaaactgggatcctgtcgatcagactgtacttgctaatgaacaagttgaaaatggtcgtggttggcgttcgggtgcattggttgaaaaacgtgatattccaatgtattacttccgtattaccgattatgcacaagaattattagacgatttagattcgcttaaagatggttggccgcaacaagtcttgaccatgcaacgcaactggattggtcgttcacaaggcatggaaatcacctttccatctgcgaaccctgaaatctatgcagatgatttaacggtttataccacacgtggtgacaccttgatgggcgtgacgtatgttgcggttgccgctgaacatccaatggcgcttaaagcggctgaaacaaatcccgaattggctgcatttattgaagaatgccgtatgggttcagtggctgaagcagatcttgccactgccgagaaaaaaggcatggccactggtttgtctgtgaagcatcctgtaacgggtgaagtggttccagtgtggattgcgaactatgtattgatgtcatacggttcaggtgcggtgatggcagttccagcacacgacgaacgtgatttcgaatttgccaacaaatatggtttaaccctccagcaagtgattgatgccaaaggtgcagacgatgctgaattttctgcaactgaatggcaggaatggtatggctcgaaagaaggcaaactggttaattctggcgaatttgacggtttagacttccaagctgcatttgatgcattcattgcaaaattagaaccacaaaaactggcaaatacgaaagttcagttccgtctacgtgactggggtgtttcgcgtcagcgttattggggttgtccaattccaatgatcaactgtgaaacttgtggtcaagtacctgtacctgaagaacaacttccagtaattttaccaactgacgtggtgccagatggttcaggcaatccgttaaataaaatgcctgaattttatgaaacccaatgtccatgttgtggtgcaggtgcacgccgtgaaaccgatactttggatacgttcgtagagtcatcttggtactatgcacgttatgcatctccagatttcactggcggtttagttaaacctgaagctgcaaaatcatggctaccagtcaaccaatatattggcggtgtggaacatgcaattttgcatttattgtatgcccgtttcttccataaattgatgcgtgatgaaggcgtcgttgaaggcaatgaacctttcgctaacttactgactcaaggtatggttttagctgataccttctaccgtgaagccgaatcaggtaagaaaacatggtttaatcctgcggatattgaattagaaaaagacgaaaaaggtcgtgttctttctgctaaatacacaggtgatggccaagaagttgtggttggcggtcaagaaaaaatgtcgaaatcgaaaaataatggcatcgacccgcaatcgattattgatcaatacggcgcagatactgcacgtgtatttatgatgtttgcggccccacccgatcaatcgcttgaatggtctgatgccggtgtggaaggtgcaaaccgtttcttgaaacgtgtatggcgtttaaccacaggtttcttagaaaaaggcaaccatgctgctgtaattgatgttgcgaatttgtcatcagcggcacaagacttacgtcgtaaaacccacgaaaccattcaaaaagtcggtgatgacattgaacgtcgtcatgccttcaatactgccattgcagcgcaaatggaattattgaatgcttgcaataaatttgaagccaaagatgataatgacgttgcggttgaacgcgatgctattgttagcttactcactttacttgcaccatttgcaccacatttaagtcagaccctattggctcaattcggtattgagttaactgaaaccttgttccctactgtggatgagtctgcgctaacccgcaacacacaaactattgtggtacaggtcaatggtaaacttcgtggcaagttggaagtgtctgttgatctctctaaagaagatattttggatcaagccaaagcattgcctgaagtacaacaattcttaaccggtccaaccaagaaagaaattgtggtgccgaataaattggtcaatttggtggtttaa

SEQ ID NO: 126 - DNA, 1671 bp, unidentified
  source 1..1671: mol_type = genomic DNA; note = DP70 Glucose-6-phosphate isomerase sequence
atgaatagtattgaaaaatttcccttgcatgatacggatctgattcaggaaaaactaaaaagttttgcccaacaagagcaagagattaatttaaattatttatttaaaaaaaataaaaaacgttttgatgaatattccgttcatgcgggtcagttatgttttgattatagtaagcaccgtgttgatgagcgtattattaacgagcttatttgttatgcggaatcacaacatttgggtaactggattcagcgcttattttctttagaaaaaattaattacactgaaaatcgcgcagcgatgcattgggctttgcgtttgccgaagcaagatagtacacatgcagatttggcagcgcaggtacatagtcagcttgatcgtatgtatcaattggtcgagaaaattcatcaggggcagtatcgaggagctacaggtgaggtcatccatgatgtggtcaatattggtgtcggtggatcagatcttggtcctttaatggtgtctcaagcgctgactgattttaaagttcaaacggctcaaaaattaaaagtccattttgtttcgacgatggatggcagccaactttcagatcttttacatcagtttcgcccagaaaccaccttgtttattatttcatccaagtcttttggcaccattgatacgctttccaatgcacaaacggcaaaatgctggcttgagcaatctttaggaacgtcgaaatcagttctaagatgtcactttgttggtgtttcaaccaagcccgataagatgaccgagtggggaatcagcactgaaaatcaattcttattgtgggattgggtcggtgggcgctattcactatggtcgtgtattggtttgcctattgcattaagtattggggtcgagggctttaaacagttgcttgctggtgcttatgaaatggatcagcattttcagaacacaccacttgaacaaaatattcctgtgttgatgggtttactgggaatatggaataacaacttcctgaatattcaaactcatgcggtacttccttatgatggtcggctgaaatattttgcggcttatttacagcaattggaaatggagtcgaatggtaagtcgattcagcgttctggtgaaaaagtcgtattagatacctgcccaattttatggggtgaagttggaccaaatgcacaacatgctttttatcagctgctgcatcaaggtacacatgctgtgagttgtgactttattgcacctgtgaaacgctataatgccaatcaatttacctatgttgaaaatgcagaggctttagttgaacaacaccatttagccttatcgaattgtttggcacaatcacgtctattggcctttggtaatcatgttctagatccgaaagaagtagaaagttcaccgaaatataaacaatatgcaggcaaccaaccgaccacaacaattttgttaaaagagttgaatccgcgcagtttaggtatgctcattgcgatgtatgagcacaaggtatttgtgcaatccgtgatgtggaatattaatccatttgaccaatggggcgtagaaaaaggtaaagaaattgccaatcaactgttaccgattctcaatcaagagcaagctgatgtttctgatcttgattcttcaacgcaaggtctattaagaattttactgggaaaagctgatggctaa

SEQ ID NO: 127 - DNA, 1788 bp, unidentified
  source 1..1788: mol_type = genomic DNA; note = DP70 NADH-quinone oxidoreductase subunit C/D sequence
atggctgaaactgacattgctatgccagaatcaacgcctgttgattcacgcccagcatttgcaattgtagaagagctcaaagccaaatttggtgagaacttctatgtgcaagcgacttttgaagattttccaacggtctgggttgagcgcgcgcgcgtacaagatgttttaatgttcttgcgtaaagtatcacgtccatacgtgatgctgttcgacttgtctgcggtagatgagcgtttacgtacccaccgtgacggtttacctgcatcagacttcactgtgttttatcatttgttgtcgctagagcgcaacagtgatattcgtattaaagttgcgttgagtgagagtgatctcaatcttccaaccgcaaccaacatttggccaaatgccaactggtacgaacgtgaagcttacgatatgttcgggatcaatttcgaagggcatccaatgctccgtcgtattttgttgccaacctattgggaaggtcacccactgcgtaaagaatattctgcacgtgcgactgaatatacaccgtatatgcagaaccaagcgaagcaggatttcgagcaagaacatttacgttttgttcctgaagattggggtctatcacgcggtaatgccgatgaagatttcatgttcttgaacttaggtccaaaccatccatctgcgcacggtgcattccgtatcattttgcagttggacggtgaagaagtgaaagactgtgtgcctgatattggctatcaccaccgtggtgtggaaaagatggctgaacgtcaaacttggcattcattcattccatataccgaccgtgttgactacttgggtggttgtgcgcaaaacatgccttatgtgatgggtgtggagcaaatggcaggaattactgttcctgaccgtgcacaatgtatccgtgtcatgatgtctgaattattccgtatcaataaccatttattgtttattggtactgcaattcaagatgccggcggtatgacgccagtcttctatatgtttgccgatcgtcaaaagatctatgatgcgattgaagcgattacaggctaccgtatgcatccagcatggttccgtattggcgggactgcgcacgaccttccaaacaattggcaacatctgattcgtgaaattctcgaatggatgccgaagcgtatgaatgaatactatacagctgcactacgcaactcagtatttattggtcgtacccgtaatgttgcacaatacgatgcaaaatctgcattggcttggggtgtaacaggtacaggtctacgcgcgacagggattgatttcgacgtgcgtaaataccgtccgtatagcggttatgaaaactacgacttcgacgtgcctttagaatacgaaggcgatgcttacgctcgtgtgatggttcacttccgtgaaattgaagaatcactgaaaattgtgaagcagtgcttggataacatgccatctggtccatataaagcggatcatcctttggctgttccaccaccaaaagacaagacattacaagatattgaaactttgattacgcacttcttgagcgtgtcatggggtcctgtgatgcctgcgggtgaagcgtctgtaatggctgaagtggtaaaaggtgcatcgaactactacttgacttcagacaagtcaaccatgagttatcgtacccgtattcgtacaccaactttcacgcacttacagcaaatgccttctgtgattaatggcagtcttgtatctgacttgatcatttatttagcgaccattgacgtcgtaatggctgacgtggatcgctag

SEQ ID NO: 128 - DNA, 1044 bp, unidentified
  source 1..1044: mol_type = genomic DNA; note = DP70 Protein RecA sequence
atggatgataataaaagtaaggcgcttaatgctgccctaagccagattgaaaaacaatttggtaaaaataccgtaatgcgtcttggtgataataccgtattggccgttgaagcggtctctacaggttctttaacactagacattgcacttggtattggtggcttaccaaaaggtcgtatcgttgaaatttacggtcctgaatcttctggtaaaaccacaatgacattgcaagcgattgcacaatgtcaaaaagccggtggtacttgtgcttttatcgatgcagaacatgcactcgatcctcagtatgcacgtaagcttggtgtcgaccttgacaacctgttggtttctcaaccagaccacggtgaacaagcccttgaaattgcagacatgttagtccgctctggtgctattgacatgatcgttgtcgattccgtggctgcactgacacctcgcgctgaaattgaaggtgaaatgggcgactcacatatgggcttacaagcacgtttgatgagtcaggcattacgtaaaattactggtaatgcaaaacgctcaaactgtatggtgatcttcattaaccaaatccgtatgaagattggtgtaatgtttggtagccctgaaaccacaacaggtggtaatgcactcaaattctacgcttctgtacgtttggatatccgtcgtattggtcaagtgaaagaaggcgatgaaattgtcggttcagaaacccgcgttaaagtcgtaaaaaataaaatggcacctccttttaaggaagcgttattccaaattttatatggcaaaggtgtcaatcaactgggtgaactggttgatcttgctgttgcgcaagaactggtacaaaaagcaggtgcttggtattcatatcaaggcaataaaattggtcaaggtaaaaacaacgtgatccgccatttagaggaaaatcctcaaattgcacaagaacttgatcgcctgattcgtgaaaaattgttgacaccaacgaccacgcctattgaagaaaaagatgaagtagaaccagactttctagatgcttaa

SEQ ID NO: 129 - DNA, 1887 bp, unidentified
  source 1..1887: mol_type = genomic DNA; note = DP70 RNA polymerase sigma factor RpoD sequence
atgagcgatatgacttcccctacttcgcaagtagcggctctgattagccgaggcaaagagcaaggttacttaacttacgctgaggttaacgatcatctcccagactcgatcacggaaagcgaacagattgaagacattattcaaatgcttcaagatgtcggcattccagtgcatgaacgtgcgcctgaatctgatgacaccatgttcgacggtaacaatgcagaagcaaccgatgaagtcgctgaagaagaagcggcagctgttcttgcttcagttgaaagcgaacctggtcgtaccaccgatccagtacgtatgtacatgcgtgaaatgggaacggttgaactattaacgcgtgaaggcgaaattagcattgcaaaacgcattgaagaaggtattcgtgacgttcttcattcgattgcgtactggccaaatgcagttgaagttgtattaaaagaatatagcgatgttgctgaaggcgaacgtcgtcttgctgatattttatctggttatttagacccagaatctgacgaagaaattccagaagttttagaagaagaagctgaaattgttgaagatgatgaagcgacgactaaaaccactaaagatgtaaaattggacgatgacgaagaagaagaatctgaaagtgatgatgattctgaaggtgagtctggtccagatccagaaattgcacgtgttcgtttcactgaattagaagatgcgtggaaagtaaccaaagccaccattgaaaagcatggccgtaacagcaaacaagcagatgaagcgcttgaagctcttgcaactgtgtttatgatgttcaaatttacaccacgtttatttgaaatcatttcagaaatgattcgtggcacgcatgaacaaattcgtacagcagaacgtgaagtgatgcgttacgcagttcgtcgtggtcgtatggaccgtacccaattccgtacatcgttcccaggccaagagtcaaatccagcttggttagatgaacaaattgctaaagcacctgcggatcaaaaaggttatttagaaaaagtacgtccagatgttgttgcattccagcaaaagattgccgatatcgaaaaagaattgggcttagatgttaaagacatcaaagacatttctaaacgtatggctgtgggtgaagcgaaagcacgtcgcgcgaaaaaagaaatggttgaagcaaacttacgtttggtgatttcgattgcgaaaaaatataccaaccgtggtttacaattccttgacttgattcaagaaggtaacatcggtttgatgaaagccgtagacaagtttgaataccgtcgtggttataaattctcgacttatgcaacttggtggattcgtcaggcgattacccgttcgattgccgatcaagcacgtaccatccgtattccagtacacatgatcgaaaccattaacaagatcaaccgtgtatctcgtcaacttcttcaagaaatgggccgtgagcctacccctgaagaattaggcgaacgtctggaaatggacgaagttaaagtacgtaaagtgctgaaaattgccaaagaaccgatttcgatggaaacaccgattggtgatgacgaagattcgcatcttggtgacttcattgaagatggtaacattacctctccaattgatgccgcgacttctgaaggcttaaaagaagcaacacgtgaagtgctggaaaacttgaccgaacgtgaagcgaaagtcttaaaaatgcgttttggtattgatatgccaaccgaccatactttagaagaagtgggtaaacaatttgatgtaacacgtgaacgtattcgtcagattgaagccaaagctttacgtaaattacgtcacccttctcgttctgaacacttacgttcattcctagaaaatgactaa

SEQ ID NO: 130 - DNA, 1671 bp, unidentified
  source 1..1671: mol_type = genomic DNA; note = DP71 Glutamine--tRNA ligase sequence
atgagtgaggctgaagcccgcccaacaaattttatccgtcagattattgatgaagatctggcgaccgggaaacacaataccgttcacacccgtttcccgcctgagcctaatggctatttgcatatcggccatgcgaagtctatctgcctgaatttcggcattgcgcaagactaccagggtcagtgcaatctgcgttttgacgatactaacccggcaaaagaagacatcgaattcgttgagtcgatcaaatacgacgtccagtggctgggcttcgactggagcggtgatattcactactcctcagactatttcgatcaactgcacgcatacgcgctggagctaatcaacaaaggtctggcgtacgttgacgaactgtctcccgatcaaattcgcgaataccgtggttcgctgaccgcaccgggcaaaaacagcccgtatcgcgatcgcagcgtggaagaaaatatcgcgctgtttgaaaaaatgcgtaacggtgaattcgccgaaggtgccgcttgcctgcgtgccaaaatcgatatggcgtcgccattcttcgtgatgcgcgatccggtcatctaccgtattaagtttgccgaacatcatcagactggcacaaaatggtgcatctacccgatgtacgatttcactcactgcatttccgatgcgctggaagggatcacccattcactgtgtacgctggaattccaggacaaccgccgtctgtacgactgggtactggataacatcactattccatgccatccgcgtcagtatgagttctcccgtctgaatcttgaatactccatcatgtccaagcgtaagctgaacctgctggtgacggataagattgtagaaggttgggacgatccgcgtatgccgacggtttccggtctgcgtcgccgtggttataccgccgcgtctatccgcgaattctgccgtcgtatcggcgtgaccaagcaggacaacaacgttgaaatgatggcgctggaatcctgtattcgtgacgatctgaacgaaaacgcaccgcgcgccatggccgttattaacccggttaaagttgtcattgagaacttcaccggtgatgacgtgcaaatggtgaaaatgccgaatcatccgagcaaaccggaaatgggcacccgcgaagtgccgttcacccgtgagatttacatcgatcaggctgatttccgcgaagaagcgaacaaacagtacaaacgtctggtgctgggcaaagaagttcgcctgcgcaatgcgtatgtgatcaaagcggaacacatcgagaaagacgcggaagggaatatcaccaccatcttctgttcttacgatatcgatacgctgagcaaagatcccgctgatggccgtaaggtgaaaggcgtgattcactgggtttctgcttctgaaggtaaaccggcagaatttcgcctgtatgaccgtctgttcagtgttgcgaaccctggccaggctgaagatttcctgaccaccatcaacccggaatctctggtgattgctcagggcttcgttgagccgtctctggtcgctgctcaggcagaagtcagtgtgcagttcgaacgtgaaggttacttctgtgccgacagccgctattcaagtgctgagcatctggtgttcaaccgcaccgtcggccttcgcgacacctgggaaagcaaacccgtcgcctga

SEQ ID NO: 131 - DNA, 2415 bp, unidentified
  source 1..2415: mol_type = genomic DNA; note = DP71 DNA gyrase subunit B sequence
atgtcgaattcttatgactcctcaagtatcaaggtattaaaagggctggacgcggtgcgtaagcgccccggcatgtatatcggcgataccgatgacggcactggtctgcaccacatggtattcgaggttgtggacaacgctatcgacgaagccctcgcgggccactgtaaagagattcaggtcacgatccatgcggataactctgtttccgtacaggatgatggtcgtggtattcctaccggcattcacgaagaagagggcgtttctgctgctcaggtcatcatgaccgtacttcatgccggcggtaaatttgacgataactcgtacaaagtctccggcggtctgcatggcgtgggtgtttccgtcgttaacgccctgtcggaaaaactggagctggttatccgccgtgaaggcaaagtgcacacccagacttacgtccacggtgagccgcaggatccgctgaaagtggttggcgataccgaggcgaccggtacgaccgtgcgcttctggccaagctacgccaccttcaccaatcaaacagaattcgagtatgacattctggcgaaacgcctccgtgagctgtcattcctgaactctggtgtggcgatccgcctgctcgacaaacgcgatggcaagaacgatcacttccattatgaaggcggtatcaaagctttcgtggaatacctgaacaaaaacaaaaccccaatccacccaaccgtgttctatttctccaccgtgaaagacgatatcggtgtggaagtggcgttgcagtggaatgatggtttccaggaaaatatttactgctttaccaacaatatccctcagcgcgacggcggcacccatctggtaggcttccgttctgcgatgacccgtacgcttaacgcgtatatggataaagaaggctacagcaagaaatccaaaatcagcgccaccggtgatgatgcccgtgaaggcctgatcgccgtggtttcggtaaaagtgccggatcctaagttctcctctcagaccaaagacaaactggtttcttccgaagtgaagaccgccgttgagtctctgatgaacgagaagctggttgattatctgatggaaaacccggccgacgcgaaaatcgttgtcggtaaaatcatcgatgcagcccgtgcgcgtgaagccgcgcgtaaagcacgtgaaatgacccgtcgtaaaggcgcgctcgatctggccggtctgccaggcaaactggctgactgtcaggaacgcgacccggcacattccgaactgtacttagtggaaggggactcagcgggcggctctgcaaaacaaggccgtaaccgtaagaaccaggcgattctgccgttgaaagggaaaatcctcaacgttgagaaagcgcgcttcgacaaaatgctctcttctcaggaagtggcgacgctgattaccgcgctcggttgcggtatcggccgtgacgaatacaacccggataaactgcgttatcacagcatcatcatcatgaccgatgccgacgtcgatggttcgcacatccgtaccctgttactgacattcttctaccgtcagatgcctgaaattgtagagcgtggccacgtgtttatcgcgcagcctccgctgtacaaagtgaaaaaaggcaaacaggaacagtacattaaagatgatgaagcgatggatcagtatcaaatctctatcgcgatggacggggcaacgttacacgccaacgcccatgcaccagcactggcgggcgaaccgctggagaaactggtggctgaacatcacagcgtgcagaaaatgattggccgtatggaacgtcgttatccgcgtgcgctgctgaataatctggtctatcagccaacgctggcgggtgctgaacttgccgacgaagcgaaagtgaaggaatggattgaaacgctggtgtctcgtctgaacgagaaagagcagcacggcagcagctacagtgcgatcgtgcgcgaaaatcttgaacaccagctgttcgagccaatcctgcgcattcgtactcacggtgtggataccgactacgatctcgatgcagacttcattcagggcggcgaataccgcaaaatctgtaccctgggtgaaaaactgcgcggcctgatcgaagaagatgcttacatcgaacgtggcgaacgccgtcagccagtgaccagcttcgagcaggcgctggaatggctggtgaaagagtcgcgtcgcggtctgtcgattcagcgttataaaggtctgggtgaaatgaaccctgagcaattgtgggaaaccacgatggatccgacacaacgccgcatgctgcgcgtgacggtgaaagatgctatcgcggcggaccagctgttcaccacgctgatgggcgatgcggttgaaccgcgccgcgccttcatcgaagagaacgcccttaaagctgccaatatcgatatctga

SEQ ID NO: 132 - DNA, 2817 bp, unidentified
  source 1..2817: mol_type = genomic DNA; note = DP71 Isoleucine--tRNA ligase sequence
atgagtgactacaagaacaccctgaatttgccggaaacagggttcccgatgcgtggcgatctggccaagcgtgaacctgacatgctgaagaattggtatgaccaggatctgtacgggattattcgtgctgccaagaaaggcaagaaaacctttatcttgcatgacggccctccgtatgcgaacggcagcattcatattggtcactcagtaaacaaaattcttaaagacatgatcgttaagtccaaaggactggcgggctttgatgcgccgtatgttccgggctgggattgtcatggtctgccgattgaactgaaagttgaacagctgatcggtaagccgggcgaaaaagtcacggcggcggaattccgtgaagcctgccgcaagtacgctgctgaacaggttgaaggtcagaagaaagacttcatccgtctgggcgtgctcggtgactgggatcatccgtacctgaccatggacttcaaaacagaagccaacatcattcgtgccctgggtaaaatcatcggcaacggtcacctgcataaaggtgcgaaacctgttcactggtgtaccgattgcggatcttcactggctgaagccgaagtcgaatattacgacaaagtgtctccgtctatcgacgtgacgtttaatgcgacggatgccgccgctgttgctgcgaaattcggtgccactgctttcaatggcccggtttctctggtcatctggaccaccaccccgtggaccatgccagctaaccgcgcgatttcactcaacgctgagttctcttatcagctggtgcagattgaaggtcagtgcctgatcctggctaccgatctggtagaaagcgtgatgaatcgcgccggtatcgctgagtggactgtgctgggcgaatgtaaaggtgcggatcttgaattgcttcgattccagcatccgttcctcggtttcgatgttccggcgatcctcggcgatcacgttactctcgatgccggtaccggtgctgtacataccgcacctggccacggtcctgatgactttgtcattggccagaaatacggtctggaagtcgcaaacccggttggaccgaacggctgctacctgccgggcacttatccgacgctggatggcaaattcgtctttaaagcgaatgatctgatcgttgaattgctgcgtgagaagggcgcactgctgcacgttgagaaaatgaaccacagctatccgtgctgctggcgtcacaaaacgccgatcatcttccgcgctacgccacaatggttcatcagcatggatcagaaaggtttgcgtcagaagtctctggaagagatcaaaggcgtgcagtggatccctgactggggtcaggcgcgtatcgaaaacatggtcgctaaccgtcctgactggtgtatctcccgccagcgtacgtggggcgtaccgatgtctctgttcgtgcataaagataccgaacagcttcatccgcgcagccttgagctgatggaagaagtggcaaaacgcgtggaagccgatggcattcaggcatggtgggatctgaaccctgaagagattttgggtgcagacgctgccgattacgtcaaagtgccggatacgctggacgtctggtttgactccggttccacgcactcctccgttgtggatgtgcgccctgagttcaacggtcattcaccggatctgtatctggaaggttctgaccagcatcgcggctggttcatgtcttctctgatgatttctacggcgatgaaaggcaaagcgccttacaaacaagtactgactcacggtttcaccgtcgatggtcagggccgtaaaatgtctaaatccatcggtaacaccatcgcgcctcaggatgtgatgaataagctgggtggcgacatcctgcgtttgtgggtggcatctacggattacaccggcgaaatcgccgtgtccgacgaaatcctcaaacgtgctgccgattcttatcgccgtatccgtaacaccgcgcgcttcctgctggcgaaccttaacggtttcgatccggcgctgcacagcgtggcaccggaagagatggttgtgctggatcgctgggcggttggccgcgcgaaagctgcacaagacgagatcattgctgcgtacgaagcctatgatttccacggcgttgttcagcgtctgatgcagttctgctcgatcgaaatgggttcgttctatctggatatcattaaagatcgccagtacaccgcgaagagcgacagcgttgcgcgccgcagctgccagaccgcgctgtatcacatctgcgaagcactggttcgctggatggcgccaatcatgtccttcactgccgatgaaatctgggctgaactgccaggtcatcgcgagaagttcgtctttactgaagaatggtacgacggtctgtttggcctgatcggtaacgaatccatgaacgatgcgttctgggatgagctgctgaaagtgcgtggtgaagtgaacaaagtgatcgaacaggcgcgtgctgataaacgtctgggcggttctctggaagcagccgtgaccttatatgcagacgacgcgctggcaacagacctgcgttctctgggtaacgaactgcgctttgtgctcctgacttccggtgcgaaagtcgccgcgctgtctgaagctgatgactcagcgcaggccagcgaattgttgaaaggactgaaaattggtctggcgaaagcagaaggcgagaagtgcccgcgctgctggcatttcaccactgatatcggccagaatgcggaacacagtgacatctgtggccgttgtgtgactaacattgccggtgacggcgaagagcgtaagtttgcataa

SEQ ID NO: 133 - DNA, 1560 bp, unidentified
  source 1..1560: mol_type = genomic DNA; note = DP71 NADH-quinone oxidoreductase subunit C/D sequence
atgtcagaacttactcatattaatgcttccggcgacgcccacatggtggatgtctccggtaaagacgacaccgttcgtgaagcccgtgccgaagcctttgttgaaatggccgaaagcacgctggcgatgatcatcggcggtaatcaccataagggtgacgtgttcgcgaccgcgcggattgccggtattcaggcagcgaagaaaacctgggatctgatcccgctgtgtcatccgctgttgctgaccaaggtggaagtgaatcttgaagcgcagccagaatttaatcgtgtacgtattgaatcccgctgccgcctgagcggtaaaaccggcgtcgagatggaagcgctgaccttcaagcctgaagactggggaatgaagcgcggcaccgaaaacgaggacttcatgttcctcaacctcggacctaaccatccgtctgcgcacggtgcgttccgcatcatcctgcagcttgatggcgaagaaattgtcgactgtgtaccggacgtcggttaccaccaccgtggtgctgagaagatgggcgagcgccagtcatggcacagctacattccatacacggaccgtatcgaatacctcggcggttgcgttaacgagatgccatacgtactggctgttgaaaaactggcgggtatcgtcgtgccggatcgcgttaacaccatccgcgtgatgctgtctgaactgttccgtatcaacagccacctgctgtacatctctacgtttattcaggacgtgggcgcgatgacgccagtgttcttcgcctttaccgatcgtcagaaaatttacgatctggtggaagcgatcaccggtttccgtatgcacccggcctggttccgtattggtggcgttgcacacgacctgccgaaaggctgggagcgtctgctgcgtgaattccttgactggatgccagcccgtctggattcctacgtcaaggcagcgctgaaaaacaccattctgattggacgttccaaaggcgtagcagcatacaacgccgatgatgcgctggcgtggggcaccaccggtgctggcctgcgtgcgaccgggatcgacttcgatgtccgcaaatggcgtccatattcaggttacgaaaacttcgattttgaagtgccggtcggcgatggcgtcagtgattgctattcccgcgtgatgctaaaagtggaagagcttcgtcagagcctgcgcattctggaacagtgctacaaaaacatgccggaaggcccgttcaaggcggatcacccgctgaccacgccgccaccgaaagagcgtacgctgcaacacatcgaaaccctgatcactcacttcctgcaagtgtcgtggggtccgatcatgcctgcgcaagaatctttccagatggttgaagccaccaaagggatcaacagctactacctgaccagtgacggcagcaccatgagctaccgcacgcgcgtccgtacgccaagcttcccgcatttgcagcagatcccgtccgtaatccgtggcagcctggtatccgacctgatcgtgtatctgggcagtatcgattttgtaatgtcagatgtggaccgctaa

SEQ ID NO: 134 - DNA, 1065 bp, unidentified
  source 1..1065: mol_type = genomic DNA; note = DP71 Protein RecA sequence
atggctattgatgagaacaagcaaaaagcgttagctgcagcactgggccagattgaaaagcaattcggtaaaggctccatcatgcgtctgggtgaagatcgctctatggacgtggaaacgatctctaccggctctttgtctctggatatcgcgttaggcgccggtggtttgccgatgggccgtatcgttgagatttatggcccggaatcctccggtaaaactacgctgacccttcaggttattgctgccgcacagcgcgaaggcaaaacctgtgcgttcatcgatgcggaacatgcacttgaccctatctacgcgaagaaattgggcgtagatatcgacaacctgttgtgttctcagccggataccggcgaacaggctctggaaatctgtgacgcgctgacccgttcaggcgcggtcgacgttatcatcgtcgactccgttgctgcactgacgccaaaagcagaaatcgaaggcgaaatcggtgactctcacatgggccttgcggcacgtatgatgagccaggcaatgcgtaagcttgccggtaacctgaaaaacgccaacaccttgctgatcttcatcaaccagatccgtatgaaaatcggtgtgatgttcggtaacccggaaaccaccaccggtggtaacgccctgaaattctacgcctctgtgcgtctggatatccgccgcatcggcgctatcaaagaaggcgacgtggtgatcggcagtgaaacgcgcgtgaaagttgtgaagaacaaaatcgctgcgcctttcaaacaggctgaattccagatcctatacggcgaaggcatcaacattaacggcgagctgatcgatttgggcgttaagcacaaactggtcgaaaaagccggtgcatggtacagctacaacggcgagaagattggtcagggtaaatctaactcctgcaactatctgaaagaaaacccgaaaatcgctgctgaactggataaaaaactgcgtgatatgttgttgagtggcactggtgaactggccgctgcaaccacagcagaacttgcagacgacgatatggaaaccagcgaagagttttaa

SEQ ID NO: 135 - DNA, 1794 bp, unidentified
  source 1..1794: mol_type = genomic DNA; note = DP71 RNA polymerase sigma factor RpoD sequence
ggtaaggagcaaggctatctgacctttgctgaggtcaatgaccatctgccggaagatatcgtcgactccgaccagatcgaagacatcatccagatgattaacgacatgggcatccaggttcttgaagaagcgccggacgccgatgatttgatgctggccgaaaaccgccctgataccgatgaagatgctgcagaagcagcggctcaggtgctttccagcgttgaatctgaaattggccgtaccaccgaccctgtgcgtatgtatatgcgcgaaatgggtaccgttgagctcctgacccgtgaaggcgaaatcgacatcgccaaacgtatcgaagacggtatcaatcaggtccagtgctccgttgctgaatatcctgaagctatcacctatttgttagagcaatatgaccgtgttgaagcaggcgaagcacgtctgtctgatttgatcaccggttttgttgatccgaacgccgaagaagaaatcgcgccgactgcgactcacgtgggttctgaactgaccactgaagagcaaaatgataccgacgacgatgaagaagacgacgacgatgctgaagacgacaacagcatcgacccggaactggcgcgtcagaagttcaccgatctgcgtgagcaacatgaagcgacccgtgccgtcatcaagaaaaatggccgtagccacaaaagcgccgcagaagaaattctgaagctgtccgatgtgtttaaacagttccgtctggtaccaaaacagttcgatttcctggtgaacagcatgcgctccatgatggatcgcgtccgtactcaggaacgtctgatcatgaaagtgtgcgttgaacagtgcaaaatgccgaagaaaaacttcgtcaatctgttcgccggtaacgaaaccagcagtacctggtttgatgctgctctggcaatgggtaaaccatggtctgagaagctgaaagaagtgaccgaagacgtgcagcgcggcctgatgaaactgcgccaaatcgaagaagaaactggcctgactatcgaacaggtaaaagacattaaccgtcgcatgtcgatcggcgaagcgaaagcacgccgcgcgaagaaagagatggttgaagcgaacttacgtctggttatctctatcgcgaagaaatacaccaaccgtggcttgcagttccttgacctgattcaggaaggtaacatcggcctgatgaaagccgttgataagtttgaatatcgccgtggttataagttctctacttatgcgacctggtggatccgtcaggctatcacccgctccatcgccgaccaggcacgtaccatccgtattccggtgcatatgattgagaccatcaacaaactcaaccgtatttcgcgccagatgttgcaggagatgggccgtgagccgacgccggaagagctggctgaacgcatgctgatgccggaagacaagatccgtaaagtgctgaaaattgctaaagagccaatctccatggaaacgccaatcggcgacgatgaagattcgcatctgggtgatttcatcgaggatactaccctcgagctgccgctggattctgcgacctctgaaagcctgcgttctgcaacgcacgacgttctggctggcctgaccgcacgtgaagcgaaagttctgcgtatgcgtttcggtatcgatatgaacactgaccacactctggaagaagtgggcaaacagttcgacgtaacccgtgaacgtatccgtcagatcgaagccaaagcgttgcgtaaactacgccacccaagccgctccgaagtgctgcgcagcttcctcgacgactag

SEQ ID NO: 136 - DNA, 2487 bp, unidentified
  source 1..2487: mol_type = genomic DNA; note = DP71 DNA-directed RNA polymerase subunit beta sequence
atggaccagaacaacccgttgtctgagatcacgcacaaacgtcgtatctctgcactgggcccgggcggtttgacccgtgaacgtgctggctttgaagttcgagacgtacacccgacgcactacggtcgcgtatgtccaatcgaaacgccagaaggtccaaacatcggtctgatcaactcattatctgtctatgcacagacaaatgagtatggtttcctggaaaccccttaccgccgtgtgcgtgaaggtatggttaccgatgaaattaactacctgtctgccatcgaagaaggcaactttgttatcgctcaggcgaactccaacctggatgacgaaggccacttcctggaagatttagtcacttgtcgtagcaaaggcgaatcaagcctgttcagccgcgaccaggttgactacatggacgtttctacccagcagatcgtatccgttggtgcttcactgattccattcctggaacacgatgacgccaaccgtgcattgatgggtgcgaacatgcaacgtcaggcagttcctactctgcgtgctgataagccgctggtaggtactggtatggaacgtgctgttgcggttgactccggtgttactgccgttgccaaacgtggtggtactgttcagtacgtagatgcatcccgtatcgttattcgtgttaacgaagaagagatgaatccaggcgaagcaggtatcgacatttataacctgactaagtacacccgttctaaccagaacacctgcatcaaccagatgccgtgtgtgaatctgggcgagccaatcgagcgcggcgacgtgctggcagatggtccgtcaacagatctgggcgaactggcactgggtcagaacatgcgtgtcgcgttcatgccttggaacggttacaacttcgaagactccatcttggtctccgaacgtgttgtgcaggaagatcgcttcacgaccatccatatccaggaactggcatgtgtgtcccgtgacacaaagttagggcctgaagagatcactgctgatatccctaacgtgggtgaagctgcgctctccaaactggatgagtccggtattgtgtatatcggtgctgaagtgaccggtggtgacattctggtcggtaaagttacgcctaaaggcgaaacccagctgactccagaagagaaactgctgcgtgcgatcttcggtgagaaagcgtctgacgttaaagattcttctctgcgtgtaccaaacggcgtttccggtacgattattgacgtgcaagtctttacccgcgatggcgtggaaaaagataagcgtgcgttagaaatcgaagaaatgcagctgaaacaggctaagaaagacctgactgaagagctgcaaattctggaagctggtctgtttgcacgtatccagtccgcgctggttgctggcggtgttgaagccgataagctgggcaaattgccacgcgatcgttggcttgaactgtcactgactgacgaagacaaacagaatcagttggaacagcttgctgaacagtacgacgaactgaaatccgagtttgagaaaaaactcgaagctaaacgtcgtaaaatcactcagggcgatgacctagcaccaggtgtgctgaaaatcgttaaagtgtacctggccgttaaacgtcagatccaacctggtgacaaaatggcaggccgccacggtaacaaaggtgttatctccaagatcaacccgatcgaagatatgccttacgatgaaaacgggactcctgttgacatcgtactgaacccgctgggcgttccatcacgtatgaacattggtcagattttagaaacccacctgggtatggccgcgaaaggtattggtgaaaaaatcaatgccatgcttaagaaacatgaagaagtttctaagctgcgcgagttcatccagcgtgcctatgatctgggcgacgacgtacgtcagaaagttgatctgaccaccttcaccgatgatgaagtattgcgtttggctgaaaacctgaaaaagggtatgccaattgcaacaccagtcttcgacggtgcgaaagagacagagatcaagcaactgcttgaaatgggcggcgtcccaacctctggccagatcacactgtttgacggccgtaccggcgagcaattcgagcgccaggttaccgtcggctacatgtacatgctgaaactgaaccacctggttgacgataagatgcatgcgcgttctaccggttcttacagccttgttactcagcagccgctgggtggtaaagctcagttcggtggtcagcgcttcggtgagatggaagtgtgggcactggaagcatacggtgccgcttataccctgcaggaaatgctgactgttaagtccgatgacgtgaacggccgtactaagatgtataaaaacatcgtagatggcgatcaccggatggaaccaggcatgccggaatcattcaacgtactgttgaaagaaatccgctctctgggtatcaacatcgagctggaagacgagtaa

SEQ ID NO: 137 - RNA, 1547 nt, unidentified
  source 1..1547: mol_type = rRNA; note = DP72 16S rRNA sequence
ttcggagagtttgatcctggctcaggacgaacgctggcggcgtgcctaatacatgcaagtcgagcggacagaagggagcttgctcccggatgttagcggcggacgggtgagtaacacgtgggtaacctgcctgtaagactgggataactccgggaaaccggagctaataccggatagttccttgaaccgcatggttcaaggatgaaagacggtttcggctgtcacttacagatggacccgcggcgcattagctagttggtggggtaatggctcaccaaggcgacgatgcgtagccgacctgagagggtgatcggccacactgggactgagacacggcccagactcctacgggaggcagcagtagggaatcttccgcaatggacgaaagtctgacggagcaacgccgcgtgagtgatgaaggttttcggatcgtaaagctctgttgttagggaagaacaagtgcgagagtaactgctcgcaccttgacggtacctaaccagaaagccacggctaactacgtgccagcagccgcggtaatacgtaggtggcaagcgttgtccggaattattgggcgtaaagggctcgcaggcggtttcttaagtctgatgtgaaagcccccggctcaaccggggagggtcattggaaactgggaaacttgagtgcagaagaggagagtggaattccacgtgtagcggtgaaatgcgtagagatgtggaggaacaccagtggcgaaggcgactctctggtctgtaactgacgctgaggagcgaaagcgtggggagcgaacaggattagataccctggtagtccacgccgtaaacgatgagtgctaagtgttagggggtttccgccccttagtgctgcagctaacgcattaagcactccgcctggggagtacggtcgcaagactgaaactcaaaggaattgacgggggcccgcacaagcggtggagcatgtggtttaattcgaagcaacgcgaagaaccttaccaggtcttgacatcctctgacaaccctagagatagggctttcccttcggggacagagtgacaggtggtgcatggttgtcgtcagctcgtgtcgtgagatgttgggttaagtcccgcaacgagcgcaacccttgatcttagttgccagcatttagttgggcactctaaggtgactgccggtgacaaaccggaggaaggtggggatgacgtcaaatcatcatgccccttatgacctgggctacacacgtgctacaatggacagaacaaagggctgcgagaccgcaaggtttagccaatcccataaatctgttctcagttcggatcgcagtctgcaactcgactgcgtgaagctggaatcgctagtaatcgcggatcagcatgccgcggtgaatacgttcccgggccttgtacacaccgcccgtcacaccacgagagtttgcaacacccgaagtcggtgaggtaacctttatggagccagccgccgaaggtggggcagatgattggggtgaagtcgtaacaaggtagccgtatcggaaggtgcggctggatcacctccttt

SEQ ID NO: 138 - RNA, 841 nt, unidentified
  source 1..841: mol_type = rRNA; note = DP73 16S rRNA sequence
aacggagagtttgatcctggctcaggacgaacgctggcggcgtgcctaatacatgcaagtcgagcggacagaagggagcttgctcccggacgttagcggcggacgggtgagtaacacgtgggcaacctgccccttagactgggataactccgggaaaccggagctaataccggataatccctttctccacctggagagagggtgaaagatggcttcggctatcactaagggatgggcccgcggcgcattagctagttggtaaggtaacggcttaccaaggcgacgatgcgtagccgacctgagagggtgatcggccacactgggactgagacacggcccagactcctacgggaggcagcagtagggaatcttccgcaatggacgaaagtctgacggagcaacgccgcgtgagtgaggaaggccttcgggtcgtaaagctctgttgtgagggaagaagcggtgccgttcgaatagggcggtaccttgacggtacctcaccagaaagccacggctaactacgtgccagcagccgcggtaatacgtaggtggcaagcgttgtccggaattattgggcgtaaagcgcgcgcaggcggcttcttaagtctgatgtgaaatctcggggctcaaccccgagcggccattggaaactggggagcttgagtgcagaagaggagagtggaattccacgtgtagcggtgaaatgcgtagagatgtggaggaacaccagtggcgaaggcgactctctggtctgtaactgacgctgaggcgcgaaagcgtggggagcaaacaggattagataccctggtagtccacgccgtaaacgatgagtgctaggtgttag

SEQ ID NO: 139 - RNA, 1105 nt, unidentified
  source 1..1105: mol_type = rRNA; note = DP74 16S rRNA sequence
gcctaatacatgcaagtcgtgcggaccttttaaaagcttgcttttaaaaggttagcggcgaacgggtgagtaacacgtgggcaacctgcctgtaagatcgggataatgccgggaaaccggggctaataccggatagttttttcctccgcatggaggaaaaaggaaagacggcttcggctgtcacttacagatgggcccgcggcgcattagcttgttggtggggtaacggctcaccaaggcaacgatgcgtagccgacctgagagggtgatcggccacattgggactgagacacggcccaaactcctacgggaggcagcagtagggaatcttccgcaatggacgaaagtctgacggagcaacgccgcgtgagtgaagaaggccttcgggtcgtaaaactctgttgccggggaagaacaagtgccgttcgaacagggcggcgccttgacggtacccggccagaaagccacggctaactacgtgccagcagccgcggtaatacgtaggtggcaagcgttgtccggaattattgggcgtaaagcgcgcgcaggcggcttcttaagtctgatgtgaaatcttgcggctcaaccgcaagcggtcattggaaactgggaggcttgagtgcagaagaggagagtggaattccacgtgtagcggtgaaatgcgtagagatgtggaggaacaccagtggcgaaggcggctctctggtctgtaactgacgctgaggcgcgaaagcgtggggagcaaacaggattagataccctggtagtccacgccgtaaacgatgagtgctaagtgttagagggtttccgccctttagtgctgcagctaacgcattaagcactccgcctggggagtacggccgcaaggctgaaactcaaaggaattgacgggggcccgcacaagcggtggagcatgtggtttaattcgaagcaacgcgaagaaccttaccaggtcttgacatcctctgacctccctggagacagggccttccccttcgggggacagagtgacaggtggtgcatggttgtcgtcagctcgtgtcgtgagatgttgggttaagtcccgcaacgagcgcaacccttgaccttagttgccagcattcag

SEQ ID NO: 140 - RNA, 1515 nt, unidentified
  source 1..1515: mol_type = rRNA; note = DP75 16S rRNA sequence
tgaagagtttgatcatggctcagattgaacgctggcggcaggcctaacacatgcaagtcgagcggtagagagaagcttgcttctcttgagagcggcggacgggtgagtaatgcctaggaatctgcctggtagtgggggataacgttcggaaacggacgctaataccgcatacgtcctacgggagaaagcaggggaccttcgggccttgcgctatcagatgagcctaggtcggattagctagttggtgaggtaatggctcaccaaggcgacgatccgtaactggtctgagaggatgatcagtcacactggaactgagacacggtccagactcctacgggaggcagcagtggggaatattggacaatgggcgaaagcctgatccagccatgccgcgtgtgtgaagaaggtcttcggattgtaaagcactttaagttgggaggaagggttgtagattaatactctgcaattttgacgttaccgacagaataagcaccggctaactctgtgccagcagccgcggtaatacagagggtgcaagcgttaatcggaattactgggcgtaaagcgcgcgtaggtggttcgttaagttggatgtgaaagccccgggctcaacctgggaactgcattcaaaactgacgagctagagtatggtagagggtggtggaatttcctgtgtagcggtgaaatgcgtagatataggaaggaacaccagtggcgaaggcgaccacctggactgatactgacactgaggtgcgaaagcgtggggagcaaacaggattagataccctggtagtccacgccgtaaacgatgtcaactagccgttggaatccttgagattttagtggcgcagctaacgcattaagttgaccgcctggggagtacggccgcaaggttaaaactcaaatgaattgacgggggcccgcacaagcggtggagcatgtggtttaattcgaagcaacgcgaagaaccttaccaggccttgacatccaatgaactttccagagatggatgggtgccttcgggaacattgagacaggtgctgcatggctgtcgtcagctcgtgtcgtgagatgttgggttaagtcccgtaacgagcgcaacccttgtccttagttaccagcacgttatggtgggcactctaaggagactgccggtgacaaaccggaggaaggtggggatgacgtcaagtcatcatggcccttacggcctgggctacacacgtgctacaatggtcggtacaaagggttgccaagccgcgaggtggagctaatcccataaaaccgatcgtagtccggatcgcagtctgcaactcgactgcgtgaagtcggaatcgctagtaatcgcgaatcagaatgtcgcggtgaatacgttcccgggccttgtacacaccgcccgtcacaccatgggagtgggttgcaccagaacgggaggacggttaccacggtgtgattcatgactggggtgaagtcgtaacaaggtagccgtaggggaacctgcggctggatcacctcctt

SEQ ID NO: 141 - RNA, 1479 nt, unidentified
  source 1..1479: mol_type = rRNA; note = DP76 16S rRNA sequence
cttgagagtttgatcctggctcagaacgaacgctggcggcaggcttaacacatgcaagtcgagcgccccgcaaggggagcggcagacgggtgagtaacgcgtgggaatctaccttttgctacggaacaacagttggaaacgactgctaataccgtatgtgcccttcgggggaaagatttatcggcaaaggatgagcccgcgttggattagctagttggtgaggtaaaggctcaccaaggcgacgatccatagctggtctgagaggatgatcagccacactgggactgagacacggcccagactcctacgggaggcagcagtggggaatattggacaatgggcgcaagcctgatccagccatgccgcgtgagtgatgaaggccctagggttgtaaagctctttcaccggtgaagataatgacggtaaccggagaagaagccccggctaacttcgtgccagcagccgcggtaatacgaagggggctagcgttgttcggatttactgggcgtaaagcgcacgtaggcggatttttaagtcaggggtgaaatcccggggctcaaccccggaactgcctttgatactggaagtcttgagtatggtagaggtgagtggaattccgagtgtagaggtgaaattcgtagatattcggaggaacaccagtggcgaaggcggctcactggaccattactgacgctgaggtgcgaaagcgtggggagcaaacaggattagataccctggtagtccacgccgtaaacgatgaatgttagccgtcggggggtttacctttcggtggcgcagctaacgcattaaacattccgcctggggagtacggtcgcaagattaaaactcaaaggaattgacgggggcccgcacaagcggtggagcatgtggtttaattcgaagcaacgcgcagaaccttaccagcccttgacataccggtcgcggacacagagatgtgtctttcagttcggctggaccggatacaggtgctgcatggctgtcgtcagctcgtgtcgtgagatgttgggttaagtcccgcaacgagcgcaaccctcgcctttagttgccagcatttagttgggcactctaaagggactgccagtgataagctggaggaaggtggggatgacgtcaagtcctcatggcccttacgggctgggctacacacgtgctacaatggtggtgacagtgggcagcaagcacgcgagtgtgagctaatctccaaaagccatctcagttcggattgcactctgcaactcgagtgcatgaagttggaatcgctagtaatcgcggatcagcatgccgcggtgaatacgttcccgggccttgtacacaccgcccgtcacaccatgggagttggttttacccgaaggcactgtgctaaccgcaaggaggcaggtgaccacggtagggtcagcgactggggtgaagtcgtaacaaggtagccgtaggggaacctgcggctggatcacctccttt

SEQ ID NO: 142 - RNA, 1549 nt, unidentified
  source 1..1549: mol_type = rRNA; note = DP77 16S rRNA sequence
tcggagagtttgatcctggctcaggatgaacgctggcggcgtgcctaatacatgcaagtcgagcgaactgattagaagcttgcttctatgacgttagcggcggacgggtgagtaacacgtgggcaacctgcctgtaagactgggataacttcgggaaaccgaagctaataccggataggatcttctccttcatgggagatgattgaaagatggtttcggctatcacttacagatgggcccgcggtgcattagctagttggtgaggtaacggctcaccaaggcaacgatgcatagccgacctgagagggtgatcggccacactgggactgagacacggcccagactcctacgggaggcagcagtagggaatcttccgcaatggacgaaagtctgacggagcaacgccgcgtgagtgatgaaggctttcgggtcgtaaaactctgttgttagggaagaacaagtacaagagtaactgcttgtaccttgacggtacctaaccagaaagccacggctaactacgtgccagcagccgcggtaatacgtaggtggcaagcgttatccggaattattgggcgtaaagcgcgcgcaggcggtttcttaagtctgatgtgaaagcccacggctcaaccgtggagggtcattggaaactggggaacttgagtgcagaagagaaaagcggaattccacgtgtagcggtgaaatgcgtagagatgtggaggaacaccagtggcgaaggcggctttttggtctgtaactgacgctgaggcgcgaaagcgtggggagcaaacaggattagataccctggtagtccacgccgtaaacgatgagtgctaagtgttagagggtttccgccctttagtgctgcagctaacgcattaagcactccgcctggggagtacggtcgcaagactgaaactcaaaggaattgacgggggcccgcacaagcggtggagcatgtggtttaattcgaagcaacgcgaagaaccttaccaggtcttgacatcctctgacaactctagagatagagcgttccccttcgggggacagagtgacaggtggtgcatggttgtcgtcagctcgtgtcgtgagatgttgggttaagtcccgcaacgagcgcaacccttgatcttagttgccagcattcagttgggcactctaaggtgactgccggtgacaaaccggaggaaggtggggatgacgtcaaatcatcatgccccttatgacctgggctacacacgtgctacaatggatggtacaaagggctgcaagaccgcgaggtcaagccaatcccataaaaccattctcagttcggattgtaggctgcaactcgcctacatgaagctggaatcgctagtaatcgcggatcagcatgccgcggtgaatacgttcccgggccttgtacacaccgcccgtcacaccacgagagtttgtaacacccgaagtcggtggagtaaccgtaaggagctagccgcctaaggtgggacagatgattggggtgaagtcgtaacaaggtagccgtatcggaaggtgcggctggatcacctccttt

SEQ ID NO: 143 - RNA, 1536 nt, unidentified
  source 1..1536: mol_type = rRNA; note = DP78 16S rRNA sequence
ttgaagagtttgatcatggctcagattgaacgctggcggcaggcctaacacatgcaagtcggacggtagcacagagagcttgctcttgggtgacgagtggcggacgggtgagtaatgtctggggatctgcccgatagagggggataaccactggaaacggtggctaataccgcataacgtcgcaagaccaaagagggggaccttcgggcctctcactatcggatgaacccagatgggattagctagtaggcggggtaatggcccacctaggcgacgatccctagctggtctgagaggatgaccagccacactggaactgagacacggtccagactcctacgggaggcagcagtggggaatattgcacaatgggcgcaagcctgatgcagccatgccgcgtgtatgaagaaggccttcgggttgtaaagtactttcagcggggaggaaggcgacggggttaataaccctgtcgattgacgttacccgcagaagaagcaccggctaactccgtgccagcagccgcggtaatacggagggtgcaagcgttaatcggaattactgggcgtaaagcgcacgcaggcggtctgttaagtcagatgtgaaatccccgggcttaacctgggaactgcatttgaaactggcaggcttgagtcttgtagaggggggtagaattccaggtgtagcggtgaaatgcgtagagatctggaggaataccggtggcgaaggcggccccctggacaaagactgacgctcaggtgcgaaagcgtggggagcaaacaggattagataccctggtagtccacgccgtaaacgatgtcgacttggaggttgttcccttgaggagtggcttccggagctaacgcgttaagtcgaccgcctggggagtacggccgcaaggttaaaactcaaatgaattgacgggggcccgcacaagcggtggagcatgtggtttaattcgatgcaacgcgaagaaccttacctactcttgacatccagcgaacttagcagagatgctttggtgccttcgggaacgctgagacaggtgctgcatggctgtcgtcagctcgtgttgtgaaatgttgggttaagtcccgcaacgagcgcaacccttatcctttgttgccagcgattcggtcgggaactcaaaggagactgccggtgataaaccggaggaaggtggggatgacgtcaagtcatcatggcccttacgagtagggctacacacgtgctacaatggcgcatacaaagagaagcgacctcgcgagagcaagcggacctcacaaagtgcgtcgtagtccggatcggagtctgcaactcgactccgtgaagtcggaatcgctagtaatcgtggatcagaatgccacggtgaatacgttcccgggccttgtacacaccgcccgtcacaccatgggagtgggttgcaaaagaagtaggtagcttaaccttcgggagggcgcttaccactttgtgattcatgactggggtgaagtcgtaacaaggtaaccgtaggggaacctgcggttggatcacctcctt

SEQ ID NO: 144 - RNA, 1532 nt, unidentified
  source 1..1532: mol_type = rRNA; note = DP79 16S rRNA sequence
tgaagagtttgatcatggctcagattgaacgctggcggcaggcctaacacatgcaagtcgagcggtagagagaagcttgcttctcttgagagcggcggacgggtgagtaatacctaggaatctgcctgatagtgggggataacgttcggaaacggacgctaataccgcatacgtcctacgggagaaagcaggggaccttcgggccttgcgctatcagatgagcctaggtcggattagctagttggtgaggtaatggctcaccaaggctacgatccgtaactggtctgagaggatgatcagtcacactggaactgagacacggtccagactcctacgggaggcagcagtggggaatattggacaatgggcgaaagcctgatccagccatgccgcgtgtgtgaagaaggtcttcggattgtaaagcactttaagttgggaggaagggcagttacctaatacgtgactgtcttgacgttaccgacagaataagcaccggctaactctgtgccagcagccgcggtaatacagagggtgcaagcgttaatcggaattactgggcgtaaagcgcgcgtaggtggtttgttaagttgaatgtgaaatccccgggctcaacctgggaactgcatccaaaactggcaagctagagtatggtagagggtagtggaatttcctgtgtagcggtgaaatgcgtagatataggaaggaacaccagtggcgaaggcgactacctggactgatactgacactgaggtgcgaaagcgtggggagcaaacaggattagataccctggtagtccacgccgtaaacgatgtcaactagccgttgggagtcttgaactcttagtggcgcagctaacgcattaagttgaccgcctggggagtacggccgcaaggttaaaactcaaatgaattgacgggggcccgcacaagcggtggagcatgtggtttaattcgaagcaacgcgaagaaccttaccaggccttgacatccaatgaactttctagagatagattggtgccttcgggaacattgagacaggtgctgcatggctgtcgtcagctcgtgtcgtgagatgttgggttaagtcccgtaacgagcgcaacccttgtccttagttaccagcacgtaatggtgggcactctaaggagactgccggtgacaaaccggaggaaggtggggatgacgtcaagtcatcatggcccttacggcctgggctacacacgtgctacaatggtcggtacaaagggttgccaagccgcgaggtggagctaatcccataaaaccgatcgtagtccggatcgcagtctgcaactcgactgcgtgaagtcggaatcgctagtaatcgtgaatcagaatgtcacggtgaatacgttcccgggccttgtacacaccgcccgtcacaccatgggagtgggttgcaccagaagtagctagtctaaccttcgggaggacggttaccacggtgtgattcatgactggggtgaagtcgtaacaaggtagccgtaggggaacctgcggctggatcacctcctt

SEQ ID NO: 145 - RNA, 1136 nt, unidentified; identical to SEQ ID NO: 54
  source 1..1136: mol_type = rRNA; note = DP80 16S rRNA sequence

SEQ ID NO: 146 - RNA, 1547 nt, unidentified
  source 1..1547: mol_type = rRNA; note = DP81 16S rRNA sequence
aacggagagtttgatcctggctcaggacgaacgctggcggcgtgcctaatacatgcaagtcgagcggacagaagggagcttgctcccggacgttagcggcggacgggtgagtaacacgtgggcaacctgccccttagactgggataactccgggaaaccggagctaataccggataatccctttctccacctggagagagggtgaaagatggcttcggctatcactaggggatgggcccgcggcgcattagctagttggtaaggtaacggcttaccaaggcgacgatgcgtagccgacctgagagggtgatcggccacactgggactgagacacggcccagactcctacgggaggcagcagtagggaatcttccgcaatggacgaaagtctgacggagcaacgccgcgtgagtgaggaaggctttcgggtcgtaaagctctgttgtgagggaagaagcggtaccgttcgaatagggcggtaccttgacggtacctcaccagaaagccacggctaactacgtgccagcagccgcggtaatacgtaggtggcaagcgttgtccggaattattgggcgtaaagcgcgcgcaggcggcttcttaagtctgatgtgaaatctcggggctcaaccccgagcggccattggaaactggggagcttgagtgcagaagaggagagtggaattccacgtgtagcggtgaaatgcgtagagatgtggaggaacaccagtggcgaaggcgactctctggtctgtaactgacgctgaggcgcgaaagcgtggggagcaaacaggattagataccctggtagtccacgccgtaaacgatgagtgctaggtgttaggggtttcgatgcccgtagtgccgaagttaacacattaagcactccgcctggggagtacggccgcaaggctgaaactcaaaggaattgacggggacccgcacaagcagtggagcatgtggtttaattcgaagcaacgcgaagaaccttaccaggtcttgacatcctttgaccacccaagagattgggcttccccttcgggggcaaagtgacaggtggtgcatggttgtcgtcagctcgtgtcgtgagatgttgggttaagtcccgcaacgagcgcaacccttgatcttagttgccagcattgagttgggcactctaaggtgactgccggtgacaaaccggaggaaggtggggatgacgtcaaatcatcatgccccttatgacctgggctacacacgtgctacaatggatggtacaaagggcagcgaaaccgcgaggtgaagccaatcccataaagccattctcagttcggattgcaggctgcaactcgcctgcatgaagccggaattgctagtaatcgcggatcagcatgccgcggtgaatacgttcccgggtcttgtacacaccgcccgtcacaccacgagagtttgtaacacccgaagtcggtgaggcaaccttttggagccagccgcctaaggtgggacaaatgattggggtgaagtcgtaacaaggtagccgtatcggaaggtgcggctggatcacctccttt

SEQ ID NO: 147 - RNA, 1547 nt, unidentified
  source 1..1547: mol_type = rRNA; note = DP82 16S rRNA sequence
aacggagagtttgatcctggctcaggacgaacgctggcggcgtgcctaatacatgcaagtcgagcggacagaagggagcttgctcccggacgttagcggcggacgggtgagtaacacgtgggcaacctgccccttagactgggataactccgggaaaccggagctaataccggataatccctttctccacctggagagagggtgaaagatggcttcggctatcactaagggatgggcccgcggcgcattagctagttggtaaggtaacggcttaccaaggcaacgatgcgtagccgacctgagagggtgatcggccacactgggactgagacacggcccagactcctacgggaggcagcagtagggaatcttccgcaatggacgaaagtctgacggagcaacgccgcgtgagtgaggaaggccttcgggtcgtaaagctctgttgtgagggaagaagcggtaccgttcgaatagggcggtaccttgacggtacctcaccagaaagccacggctaactacgtgccagcagccgcggtaatacgtaggtggcaagcgttgtccggaattattgggcgtaaagcgcgcgcaggcggcttcttaagtctgatgtgaaatctcggggctcaaccccgagcggccattggaaactggggagcttgagtgcagaagaggagagtggaattccacgtgtagcggtgaaatgcgtagagatgtggaggaacaccagtggcgaaggcgactctctggtctgtaactgacgctgaggcgcgaaagcgtggggagcaaacaggattagataccctggtagtccacgccgtaaacgatgagtgctaggtgttaggggtttcgatgcccgtagtgccgaagttaacacattaagcactccgcctggggagtacggccgcaaggctgaaactcaaaggaattgacggggacccgcacaagcagtggagcatgtggtttaattcgaagcaacgcgaagaaccttaccaggtcttgacatcctttgaccacccaagagattgggcttccccttcgggggcaaagtgacaggtggtgcatggttgtcgtcagctcgtgtcgtgagatgttgggttaagtcccgcaacgagcgcaacccttgatcttagttgccagcattcagttgggcactctaaggtgactgccggtgacaaaccggaggaaggtggggatgacgtcaaatcatcatgccccttatgacctgggctacacacgtgctacaatggatggtacaaagggcagcgaaaccgcgaggtgaagccaatcccataaagccattctcagttcggattgcaggctgcaactcgcctgcatgaagccggaattgctagtaatcgcggatcagcatgccgcggtgaatacgttcccgggtcttgtacacaccgcccgtcacaccacgagagtttgtaacacccgaagtcggtgaggcaaccttttggagccagccgcctaaggtgggacaaatgattggggtgaagtcgtaacaaggtagccgtatcggaaggtgcggctggatcacctccttt

SEQ ID NO: 148 - RNA, 1548 nt, unidentified
  source 1..1548: mol_type = rRNA; note = DP83 16S rRNA sequence
acggagagtttgatcctggctcaggacgaacgctggcggcgtgcctaatacatgcaagtcgagcggagtttcaagaagcttgctttttgaaacttagcggcggacgggtgagtaacacgtgggcaacctgccccttagactgggataactccgggaaaccggagctaataccggataatccctttctccacctggagagagggtgaaagatggcttcggctatcactaagggatgggcccgcggcgcattagctagttggtaaggtaacggcttaccaaggcaacgatgcgtagccgacctgagagggtgatcggccacactgggactgagacacggcccagactcctacgggaggcagcagtagggaatcttccgcaatggacgaaagtctgacggagcaacgccgcgtgagtgaggaaggccttcgggtcgtaaagctctgttgtgagggaagaagcggtaccgttcgaatagggcggtaccttgacggtacctcaccagaaagccacggctaactacgtgccagcagccgcggtaatacgtaggtggcaagcgttgtccggaattattgggcgtaaagcgcgcgcaggcggcttcttaagtctgatgtgaaatctcggggctcaaccccgagcggccattggaaactggggagcttgagtgcagaagaggagagtggaattccacgtgtagcggtgaaatgcgtagagatgtggaggaacaccagtggcgaaggcgactctctggtctgtaactgacgctgaggcgcgaaagcgtggggagcaaacaggattagataccctggtagtccacgccgtaaacgatgagtgctaggtgttaggggtttcgatgcccgtagtgccgaagttaacacattaagcactccgcctggggagtacggccgcaaggctgaaactcaaaggaattgacggggacccgcacaagcagtggagcatgtggtttaattcgaagcaacgcgaagaaccttaccaggtcttgacatcctttgaccacccaagagattgggcttccccttcgggggcaaagtgacaggtggtgcatggttgtcgtcagctcgtgtcgtgagatgttgggttaagtcccgcaacgagcgcaacccttgatcttagttgccagcattcagttgggcactctaaggtgactgccggtgacaaaccggaggaaggtggggatgacgtcaaatcatcatgccccttatgacctgggctacacacgtgctacaatggatggtacaaagggcagcgaagccgcgaggtgaagccaatcccataaagccattctcagttcggattgcaggctgcaactcgcctgcatgaagccggaattgctagtaatcgcggatcagcatgccgcggtgaatacgttcccgggtcttgtacacaccgcccgtcacaccacgagagtttgtaacacccgaagtcggtgaggcaaccttttggagccagccgcctaaggtgggacaaatgattggggtgaagtcgtaacaaggtagccgtatcggaaggtgcggctggatcacctccttt

SEQ ID NO: 149 - RNA, 1480 nt, unidentified
  source 1..1480: mol_type = rRNA; note = DP84 16S rRNA sequence
tacggagagtttgatcctggctcaggatgaacgctggcggcgtgcttaacacatgcaagtcgaacggtgaagccaagcttgcttggtggatcagtggcgaacgggtgagtaacacgtgagcaacctgccctggactctgggataagcgctggaaacggcgtctaatactggatatgagctctcatcgcatggtgggggttggaaagattttttggtctgggatgggctcgcggcctatcagcttgttggtgaggtaatggctcaccaaggcgtcgacgggtagccggcctgagagggtgaccggccacactgggactgagacacggcccagactcctacgggaggcagcagtggggaatattgcacaatgggcgaaagcctgatgcagcaacgccgcgtgagggatgacggccttcgggttgtaaacctcttttagcagggaagaagcgaaagtgacggtacctgcagaaaaagcgccggctaactacgtgccagcagccgcggtaatacgtagggcgcaagcgttatccggaattattgggcgtaaagagctcgtaggcggtttgtcgcgtctgctgtgaaatcccgaggctcaacctcgggcctgcagtgggtacgggcagactagagtgcggtaggggagattggaattcctggtgtagcggtggaatgcgcagatatcaggaggaacaccgatggcgaaggcagatctctgggccgtaactgacgctgaggagcgaaagggtggggagcaaacaggcttagataccctggtagtccaccccgtaaacgttgggaactagttgtggggaccattccacggtttccgtgacgcagctaacgcattaagttccccgcctggggagtacggccgcaaggctaaaactcaaaggaattgacggggacccgcacaagcggcggagcatgcggattaattcgatgcaacgcgaagaaccttaccaaggcttgacatacaccagaacgggccagaaatggtcaactctttggacactggtgaacaggtggtgcatggttgtcgtcagctcgtgtcgtgagatgttgggttaagtcccgcaacgagcgcaaccctcgttctatgttgccagcacgtaatggtgggaactcatgggatactgccggggtcaactcggaggaaggtggggatgacgtcaaatcatcatgccccttatgtcttgggcttcacgcatgctacaatggccggtacaaagggctgcaataccgtgaggtggagcgaatcccaaaaagccggtcccagttcggattgaggtctgcaactcgacctcatgaagtcggagtcgctagtaatcgcagatcagcaacgctgcggtgaatacgttcccgggtcttgtacacaccgcccgtcaagtcatgaaaggagccgtcgaaggtgggatcggtaattaggactaagtcgtaacaaggtagccgtaccggaaggtgcggctggatcacctccttt

SEQ ID NO: 150 - RNA, 924 nt, unidentified
  source 1..924: mol_type = rRNA; note = DP85 16S rRNA sequence
acggtcgggggcatcagtattcagtcgtcagaggtgaaattcttggattgactgaagactaactactgcgaaagcatttgccaaggacgttttcattaatcaagaacgaaagttaggggatcgaagatgatcagataccgtcgtagtcttaaccataaactatgccgactagagatcgggtggtgctttttgcgcactcggcatcttacgagaaatcaaagtctttgggttctggggggagtatggtcgcaaggctgaaacttaaaggaattgacggaggggcaccaccaggagtggagcctgcggcttaatttgactcaacacggggaaactcaccaggtccagacgtaataaggattgacaagttagagacttctcttgatcttacgggtggtggtgcatggccgtttttagtccttggagtgatttgtctgcttaattgcgataacggacgagaccttaacctgctaaatagggctgcgagcatctgctcgtgggctcttcttagagggactatgggtatcaaacccatggaagtttgaggcaacaacaggtctgtgatgcccttagacgttctgggccgcacgcgcgctacactgacggagccagcaagcataaccttggtcgagaggcctgggtaatctcgtgaaactccgtcgtgctggggatagagcattgtaatttttgctcttcaacgaggaattcctagtaagcgcaagtcatcagcttgcgttgattacgtccctgccccttgtacacaccgcccgtcgctactaccgattgaatggcttagtgaggcttcaagaccggcgcggcctgcggggcaactcgcgcgctgcgctgggaatttagtcaaacttggtcatttagaggtcgtaaaagtcgtaacaaggtttccgtaggtgaacctgcggaaggatcatt

SEQ ID NO: 151 - RNA, 881 nt, unidentified
  source 1..881: mol_type = rRNA; note = DP86 16S rRNA sequence
cgatgcgtagccgacctgagagggtgatcggccacactgggactgagacacggcccagactcctacgggaggcagcagtagggaatcttccgcaatggacgaaagtctgacggagcaacgccgcgtgagtgatgaaggttttcggatcgtaaagctctgttgttagggaagaacaagtgccgttcaaatagggcggcaccttgacggtacctaaccagaaagccacggctaactacgtgccagcagccgcggtaatacgtaggtggcaagcgttgtccggaattattgggcgtaaagggctcgcaggcggtttcttaagtctgatgtgaaagcccccggctcaaccggggagggtcattggaaactggggaacttgagtgcagaagaggagagtggaattccacgtgtagcggtgaaatgcgtagagatgtggaggaacaccagtggcgaaggcgactctctggtctgtaactgacgctgaggagcgaaagcgtggggagcgaacaggattagataccctggtagtccacgccgtaaacgatgagtgctaagtgttagggggtttccgccccttagtgctgcagctaacgcattaagcactccgcctggggagtacggtcgcaagactgaaactcaaaggaattgacgggggcccgcacaagcggtggagcatgtggtttaattcgaagcaacgcgaagaaccttaccaggtcttgacatcctctgacaatcctagagataggacgtccccttcgggggcagagtgacaggtggtgcatggttgtcgtcagctcgtgtcgtgagatgttgggttaagtcccgcaacgagcgcaacccttgatcttagttgccagcattcagttgggtgttctttgaaaact

SEQ ID NO: 152 - RNA, 1563 nt, unidentified
  source 1..1563: mol_type = rRNA; note = DP87 16S rRNA sequence
tttgagagtttgatcctggctcaggacgaacgctggcggcgtgcctaatacatgcaagtcgaacgaactctggtattgattggtgcttgcatcatgatttacatttgagtgagtggcgaactggtgagtaacacgtgggaaacctgcccagaagcgggggataacacctggaaacagatgctaataccgcataacaacttggaccgcatggtccgagcttgaaagatggcttcggctatcacttttggatggtcccgcggcgtattagctagatggtggggtaacggctcaccatggcaatgatacgtagccgacctgagagggtaatcggccacattgggactgagacacggcccaaactcctacgggaggcagcagtagggaatcttccacaatggacgaaagtctgatggagcaacgccgcgtgagtgaagaagggtttcggctcgtaaaactctgttgttaaagaagaacatatctgagagtaactgttcaggtattgacggtatttaaccagaaagccacggctaactacgtgccagcagccgcggtaatacgtaggtggcaagcgttgtccggatttattgggcgtaaagcgagcgcaggcggttttttaagtctgatgtgaaagccttcggctcaaccgaagaagtgcatcggaaactgggaaacttgagtgcagaagaggacagtggaactccatgtgtagcggtgaaatgcgtagatatatggaagaacaccagtggcgaaggcggctgtctggtctgtaactgacgctgaggctcgaaagtatgggtagcaaacaggattagataccctggtagtccataccgtaaacgatgaatgctaagtgttggagggtttccgcccttcagtgctgcagctaacgcattaagcattccgcctggggagtacggccgcaaggctgaaactcaaaggaattgacgggggcccgcacaagcggtggagcatgtggtttaattcgaagctacgcgaagaaccttaccaggtcttgacatactatgcaaatctaagagattagacgttcccttcggggacatggatacaggtggtgcatggttgtcgtcagctcgtgtcgtgagatgttgggttaagtcccgcaacgagcgcaacccttattatcagttgccagcattaagttgggcactctggtgagactgccggtgacaaaccggaggaaggtggggatgacgtcaaatcatcatgccccttatgacctgggctacacacgtgctacaatggatggtacaacgagttgcgaactcgcgagagtaagctaatctcttaaagccattctcagttcggattgtaggctgcaactcgcctacatgaagtcggaatcgctagtaatcgcggatcagcatgccgcggtgaatacgttcccgggccttgtacacaccgcccgtcacaccatgagagtttgtaacacccaaagtcggtggggtaaccttttaggaaccagccgcctaaggtgggacagatgattagggtgaagtcgtaacaaggtagccgtaggagaacctgcggctggatcacctcctt

SEQ ID NO: 153 - RNA, 1546 nt, unidentified
  source 1..1546: mol_type = rRNA; note = DP88 16S rRNA sequence
tagtgggtttgatcctggctcaggacgaacgctggcggcgtgcctaatacatgcaagtcgagcggacagatgggagcttgctccctgatgttagcggcggacgggtgagtaacacgtgggtaacctgcctgtaagactgggataactccgggaaaccggggctaataccggatggttgtctgaaccgcatggttcagacataaaaggtggcttcggctaccacttacagatggacccgcggcgcattagctagttggtgaggtaacggctcaccaaggcgacgatgcgtagccgacctgagagggtgatcggccacactgggactgagacacggcccagactcctacgggaggcagcagtagggaatcttccgcaatggacgaaagtctgacggagcaacgccgcgtgagtgatgaaggttttcggatcgtaaagctctgttgttagggaagaacaagtgccgttcaaatagggcggcaccttgacggtacctaaccagaaagccacggctaactacgtgccagcagccgcggtaatacgtaggtggcaagcgttgtccggaattattgggcgtaaagggctcgcaggcggtttcttaagtctgatgtgaaagcccccggctcaaccggggagggtcattggaaactggggaacttgagtgcagaagaggagagtggaattccacgtgtagcggtgaaatgcgtagagatgtggaggaacaccagtggcgaaggcgactctctggtctgtaactgacgctgaggagcgaaagcgtggggagcgaacaggattagataccctggtagtccacgccgtaaacgatgagtgctaagtgttagggggtttccgccccttagtgctgcagctaacgcattaagcactccgcctggggagtacggtcgcaagactgaaactcaaaggaattgacgggggcccgcacaagcggtggagcatgtggtttaattcgaagcaacgcgaagaaccttaccaggtcttgacatcctctgacaatcctagagataggacgtccccttcgggggcagagtgacaggtggtgcatggttgtcgtcagctcgtgtcgtgagatgttgggttaagtcccgcaacgagcgcaacccttgatcttagttgccagcattcagttgggcactctaaggtgactgccggtgacaaaccggaggaaggtggggatgacgtcaaatcatcatgccccttatgacctgggctacacacgtgctacaatggacagaacaaagggcagcgaaaccgcgaggttaagccaatcccacaaatctgttctcagttcggatcgcagtctgcaactcgactgcgtgaagctggaatcgctagtaatcgcggatcagcatgccgcggtgaatacgttcccgggccttgtacacaccgcccgtcacaccacgagagtttgtaacacccgaagtcggtgaggtaacctttatggagccagccgccgaaggtgggacagatgattggggtgaagtcgtaacaaggtagccgtatcggaaggtgcggctggatcacctccttt

SEQ ID NO: 154 - RNA, 1285 nt, unidentified
  source 1..1285: mol_type = rRNA; note = DP89 16S rRNA sequence
gtaacggctcaccaaggcaacgatgcgtagccgacctgagagggtgatcggccacactgggactgagacacggcccagactcctacgggaggcagcagtagggaatcttccgcaatggacgaaagtctgacggagcaacgccgcgtgagtgatgaaggttttcggatcgtaaagctctgttgttagggaagaacaagtaccgttcgaatagggcggtaccttgacggtacctaaccagaaagccacggctaactacgtgccagcagccgcggtaatacgtaggtggcaagcgttgtccggaattattgggcgtaaagggctcgcaggcggtttcttaagtctgatgtgaaagcccccggctcaaccggggagggtcattggaaactggggaacttgagtgcagaagaggagagtggaattccacgtgtagcggtgaaatgcgtagagatgtggaggaacaccagtggcgaaggcgactctctggtctgtaactgacgctgaggagcgaaagcgtggggagcgaacaggattagataccctggtagtccacgccgtaaacgatgagtgctaagtgttagggggtttccgccccttagtgctgcagctaacgcattaagcactccgcctggggagtacggtcgcaagactgaaactcaaaggaattgacgggggcccgcacaagcggtggagcatgtggtttaattcgaagcaacgcgaagaaccttaccaggtcttgacatcctctgacaatcctagagataggacgtccccttcgggggcagagtgacaggtggtgcatggttgtcgtcagctcgtgtcgtgagatgttgggttaagtcccgcaacgagcgcaacccttgatcttagttgccagcattcagttgggcactctaaggtgactgccggtgacaaaccggaggaaggtggggatgacgtcaaatcatcatgccccttatgacctgggctacacacgtgctacaatggacagaacaaagggcagcgaaaccgcgaggttaagccaatcccacaaatctgttctcagttcggatcgcagtctgcaactcgactgcgtgaagctggaatcgctagtaatcgcggatcagcatgccgcggtgaatacgttcccgggccttgtacacaccgcccgtcacaccacgagagtttgtaacacccgaagtcggtgaggtaaccttttaggagccagccgccgaaggtgggacagatgattggggtgaagtcgtaacaaggtagccgtatcggaaggtgcggctggatcacctccttt

SEQ ID NO: 155 - RNA, 1563 nt, unidentified; identical to SEQ ID NO: 152
  source 1..1563: mol_type = rRNA; note = DP90 16S rRNA sequence

SEQ ID NO: 156 - RNA, 1265 nt, unidentified
  source 1..1265: mol_type = rRNA; note = DP92 16S rRNA sequence
cgatgcgtagccgacctgagagggtgatcggccacactgggactgagacacggcccagactcctacgggaggcagcagtagggaatcttccgcaatggacgaaagtctgacggagcaacgccgcgtgagtgatgaaggttttcggatcgtaaagctctgttgttagggaagaacaagtaccgttcgaatagggcggtaccttgacggtacctaaccagaaagccacggctaactacgtgccagcagccgcggtaatacgtaggtggcaagcgttgtccggaattattgggcgtaaagggctcgcaggcggtttcttaagtctgatgtgaaagcccccggctcaaccggggagggtcattggaaactggggaacttgagtgcagaagaggagagtggaattccacgtgtagcggtgaaatgcgtagagatgtggaggaacaccagtggcgaaggcgactctctggtctgtaactgacgctgaggagcgaaagcgtggggagcgaacaggattagataccctggtagtccacgccgtaaacgatgagtgctaagtgttagggggtttccgccccttagtgctgcagctaacgcattaagcactccgcctggggagtacggtcgcaagactgaaactcaaaggaattgacgggggcccgcacaagcggtggagcatgtggtttaattcgaagcaacgcgaagaaccttaccaggtcttgacatcctctgacaatcctagagataggacgtccccttcgggggcagagtgacaggtggtgcatggttgtcgtcagctcgtgtcgtgagatgttgggttaagtcccgcaacgagcgcaacccttgatcttagttgccagcattcagttgggcactctaaggtgactgccggtgacaaaccggaggaaggtggggatgacgtcaaatcatcatgccccttatgacctgggctacacacgtgctacaatggacagaacaaagggcagcgaaaccgcgaggttaagccaatcccacaaatctgttctcagttcggatcgcagtctgcaactcgactgcgtgaagctggaatcgctagtaatcgcggatcagcatgccgcggtgaatacgttcccgggccttgtacacaccgcccgtcacaccacgagagtttgtaacacccgaagtcggtgaggtaaccttttaggagccagccgccgaaggtgggacagatgattggggtgaagtcgtaacaaggtagccgtatcggaaggtgcggctggatcacctccttt

SEQ ID NO: 157 - RNA, 1548 nt, unidentified
  source 1..1548: mol_type = rRNA; note = DP93 16S rRNA sequence
attgagagtttgatcctggctcaggatgaacgctggcggcgtgcctaatacatgcaagtcgaacgcacagcgaaaggtgcttgcacctttcaagtgagtggcgaacgggtgagtaacacgtggacaacctgcctcaaggctggggataacatttggaaacagatgctaataccgaataaaacttagtgtcgcatgacaaaaagttaaaaggcgcttcggcgtcacctagagatggatccgcggtgcattagttagttggtggggtaaaggcctaccaagacaatgatgcatagccgagttgagagactgatcggccacattgggactgagacacggcccaaactcctacgggaggctgcagtagggaatcttccacaatgggcgaaagcctgatggagcaacgccgcgtgtgtgatgaaggctttcgggtcgtaaagcactgttgtatgggaagaacagctagaataggaaatgattttagtttgacggtaccataccagaaagggacggctaaatacgtgccagcagccgcggtaatacgtatgtcccgagcgttatccggatttattgggcgtaaagcgagcgcagacggtttattaagtctgatgtgaaagcccggagctcaactccggaatggcattggaaactggttaacttgagtgcagtagaggtaagtggaactccatgtgtagcggtggaatgcgtagatatatggaagaacaccagtggcgaaggcggcttactggactgcaactgacgttgaggctcgaaagtgtgggtagcaaacaggattagataccctggtagtccacaccgtaaacgatgaacactaggtgttaggaggtttccgcctcttagtgccgaagctaacgcattaagtgttccgcctggggagtacgaccgcaaggttgaaactcaaaggaattgacggggacccgcacaagcggtggagcatgtggtttaattcgaagcaacgcgaagaaccttaccaggtcttgacatcctttgaagcttttagagatagaagtgttctcttcggagacaaagtgacaggtggtgcatggtcgtcgtcagctcgtgtcgtgagatgttgggttaagtcccgcaacgagcgcaacccttattgttagttgccagcattcagatgggcactctagcgagactgccggtgacaaaccggaggaaggcggggacgacgtcagatcatcatgccccttatgacctgggctacacacgtgctacaatggcgtatacaacgagttgccaacccgcgagggtgagctaatctcttaaagtacgtctcagttcggattgtagtctgcaactcgactacatgaagtcggaatcgctagtaatcgcggatcagcacgccgcggtgaatacgttcccgggtcttgtacacaccgcccgtcacaccatgggagtttgtaatgcccaaagccggtggcctaaccttttaggaaggagccgtctaaggcaggacagatgactggggtgaagtcgtaacaaggtagccgtaggagaacctgcggctggatcacctccttt

SEQ ID NO: 158 - RNA, 1421 nt, unidentified
  source 1..1421: mol_type = rRNA; note = DP94 16S rRNA sequence
atctgcccagaagcaggggataacacttggaaacaggtgctaataccgtataacaacaaaatccgcatggattttgtttgaaaggtggcttcggctatcacttctggatgatcccgcggcgtattagttagttggtgaggtaaaggcccaccaagacgatgatacgtagccgacctgagagggtaatcggccacattgggactgagacacggcccaaactcctacgggaggcagcagtagggaatcttccacaatggacgaaagtctgatggagcaatgccgcgtgagtgaagaagggtttcggctcgtaaaactctgttgttaaagaagaacacctttgagagtaactgttcaagggttgacggtatttaaccagaaagccacggctaactacgtgccagcagccgcggtaatacgtaggtggcaagcgttgtccggatttattgggcgtaaagcgagcgcaggcggttttttaagtctgatgtgaaagccttcggcttaaccggagaagtgcatcggaaactgggagacttgagtgcagaagaggacagtggaactccatgtgtagcggtggaatgcgtagatatatggaagaacaccagtggcgaaggcggctgtctagtctgtaactgacgctgaggctcgaaagcatgggtagcgaacaggattagataccctggtagtccatgccgtaaacgatgagtgctaagtgttggagggtttccgcccttcagtgctgcagctaacgcattaagcactccgcctggggagtacgaccgcaaggttgaaactcaaaggaattgacgggggcccgcacaagcggtggagcatgtggtttaattcgaagctacgcgaagaaccttaccaggtcttgacatcttctgccaatcttagagataagacgttcccttcggggacagaatgacaggtggtgcatggttgtcgtcagctcgtgtcgtgagatgttgggttaagtcccgcaacgagcgcaacccttattatcagttgccagcattcagttgggcactctggtgagactgccggtgacaaaccggaggaaggtggggatgacgtcaaatcatcatgccccttatgacctgggctacacacgtgctacaatggacggtacaacgagttgcgaagtcgtgaggctaagctaatctcttaaagccgttctcagttcggattgtaggctgcaactcgcctacatgaagttggaatcgctagtaatcgcggatcagcatgccgcggtgaatacgttcccgggccttgtacacaccgcccgtcacaccatgagagtttgtaacacccaaagccggtgagataaccttcgggagtcagccgtctaaggtgggacagatgattagggtgaagtcgtaacaaggtagccgtaggagaacctgcggctggatcacctcctt

SEQ ID NO: 159 - RNA, 1388 nt, unidentified
  source 1..1388: mol_type = rRNA; note = DP95 16S rRNA sequence
tgctaataccgcatagatccaagaaccgcatggttcttggctgaaagatggcgtaagctatcgcttttggatggacccgcggcgtattagctagttggtgaggtaatggctcaccaaggcgatgatacgtagccgaactgagaggttgatcggccacattgggactgagacacggcccaaactcctacgggaggcagcagtagggaatcttccacaatggacgcaagtctgatggagcaacgccgcgtgagtgaagaaggctttcgggtcgtaaaactctgttgttggagaagaatggtcggcagagtaactgttgtcggcgtgacggtatccaaccagaaagccacggctaactacgtgccagcagccgcggtaatacgtaggtggcaagcgttatccggatttattgggcgtaaagcgagcgcaggcggttttttaagtctgatgtgaaagccctcggcttaaccgaggaagcgcatcggaaactgggaaacttgagtgcagaagaggacagtggaactccatgtgtagcggtgaaatgcgtagatatatggaagaacaccagtggcgaaggcggctgtctggtctgtaactgacgctgaggctcgaaagcatgggtagcgaacaggattagataccctggtagtccatgccgtaaacgatgaatgctaggtgttggagggtttccgcccttcagtgccgcagctaacgcattaagcattccgcctggggagtacgaccgcaaggttgaaactcaaaggaattgacgggggcccgcacaagcggtggagcatgtggtttaattcgaagcaacgcgaagaaccttaccaggtcttgacatcttttgatcacctgagagatcaggtttccccttcgggggcaaaatgacaggtggtgcatggttgtcgtcagctcgtgtcgtgagatgttgggttaagtcccgcaacgagcgcaacccttatgactagttgccagcatttagttgggcactctagtaagactgccggtgacaaaccggaggaaggtggggatgacgtcaaatcatcatgccccttatgacctgggctacacacgtgctacaatggatggtacaacgagttgcgagaccgcgaggtcaagctaatctcttaaagccattctcagttcggactgtaggctgcaactcgcctacacgaagtcggaatcgctagtaatcgcggatcagcacgccgcggtgaatacgttcccgggccttgtacacaccgcccgtcacaccatgagagtttgtaacacccgaagccggtggcgtaacccttttagggagcgagccgtctaaggtgggacaaatgattagggtgaagtcgtaacaaggtagccgtaggagaacctgcggctggatcacctccttt

SEQ ID NO: 160 - RNA, 1219 nt, unidentified
  source 1..1219: mol_type = rRNA; note = DP96 16S rRNA sequence
acacggcccaaactcctacgggaggcagcagtagggaatcttccacaatggacgcaagtctgatggagcaacgccgcgtgagtgaagaaggctttcgggtcgtaaaactctgttgttggagaagaatggtcggcagagtaactgttgtcggcgtgacggtatccaaccagaaagccacggctaactacgtgccagcagccgcggtaatacgtaggtggcaagcgttatccggatttattgggcgtaaagcgagcgcaggcggttttttaagtctgatgtgaaagccctcggcttaaccgaggaagcgcatcggaaactgggaaacttgagtgcagaagaggacagtggaactccatgtgtagcggtgaaatgcgtagatatatggaagaacaccagtggcgaaggcggctgtctggtctgtaactgacgctgaggctcgaaagcatgggtagcgaacaggattagataccctggtagtccatgccgtaaacgatgaatgctaggtgttggagggtttccgcccttcagtgccgcagctaacgcattaagcattccgcctggggagtacgaccgcaaggttgaaactcaaaggaattgacgggggcccgcacaagcggtggagcatgtggtttaattcgaagcaacgcgaagaaccttaccaggtcttgacatcttttgatcacctgagagatcaggtttccccttcgggggcaaaatgacaggtggtgcatggttgtcgtcagctcgtgtcgtgagatgttgggttaagtcccgcaacgagcgcaacccttatgactagttgccagcatttagttgggcactctagtaagactgccggtgacaaaccggaggaaggtggggatgacgtcaaatcatcatgccccttatgacctgggctacacacgtgctacaatggatggtacaacgagttgcgagaccgcgaggtcaagctaatctcttaaagccattctcagttcggactgtaggctgcaactcgcctacacgaagtcggaatcgctagtaatcgcggatcagcacgccgcggtgaatacgttcccgggccttgtacacaccgcccgtcacaccatgagagtttgtaacacccgaagccggtggcgtaacccttttagggagcgagccgtctaaggtgggacaaatgattagggtgaagtcgtaacaaggtagccgtaggagaacctgcggctggatcacctccttt

SEQ ID NO: 161 - RNA, 1546 nt, unidentified
  source 1..1546: mol_type = rRNA; note = DP97 16S rRNA sequence
aatgagagtttgatcctggctcaggacgaacgctggcggcgtgcctaatacatgcaagtcgagcgatgattaaagatagcttgctatttttatgaagagcggcgaacgggtgagtaacgcgtgggaaatctgccgagtagcgggggacaacgtttggaaacgaacgctaataccgcataacaatgagaatcgcatgattcttatttaaaagaagcaattgcttcactacttgatgatcccgcgttgtattagctagttggtagtgtaaaggactaccaaggcgatgatacatagccgacctgagagggtgatcggccacactgggactgagacacggcccagactcctacgggaggcagcagtagggaatcttcggcaatgggggcaaccctgaccgagcaacgccgcgtgagtgaagaaggttttcggatcgtaaaactctgttgttagagaagaacgttaagtagagtggaaaattacttaagtgacggtatctaaccagaaagggacggctaactacgtgccagcagccgcggtaatacgtaggtcccaagcgttgtccggatttattgggcgtaaagcgagcgcaggtggtttcttaagtctgatgtaaaaggcagtggctcaaccattgtgtgcattggaaactgggagacttgagtgcaggagaggagagtggaattccatgtgtagcggtgaaatgcgtagatatatggaggaacaccggaggcgaaagcggctctctggcctgtaactgacactgaggctcgaaagcgtggggagcaaacaggattagataccctggtagtccacgccgtaaacgatgagtgctagctgtagggagctataagttctctgtagcgcagctaacgcattaagcactccgcctggggagtacgaccgcaaggttgaaactcaaaggaattgacgggggcccgcacaagcggtggagcatgtggtttaattcgaagcaacgcgaagaaccttaccaggtcttgacatactcgtgatatccttagagataaggagttccttcgggacacgggatacaggtggtgcatggttgtcgtcagctcgtgtcgtgagatgttgggttaagtcccgcaacgagcgcaacccttattactagttgccatcattaagttgggcactctagtgagactgccggtgataaaccggaggaaggtggggatgacgtcaaatcatcatgccccttatgacctgggctacacacgtgctacaatggatggtacaacgagtcgccaacccgcgagggtgcgctaatctcttaaaaccattctcagttcggattgcaggctgcaactcgcctgcatgaagtcggaatcgctagtaatcgcggatcagcacgccgcggtgaatacgttcccgggccttgtacacaccgcccgtcacaccacggaagttgggagtacccaaagtaggttgcctaaccgcaaggagggcgcttcctaaggtaagaccgatgactggggtgaagtcgtaacaaggtagccgtatcggaaggtgcggctggatcacctccttt

SEQ ID NO: 162 - RNA, 1546 nt, unidentified; identical to SEQ ID NO: 161
  source 1..1546: mol_type = rRNA; note = DP98 16S rRNA sequence

SEQ ID NO: 163 - RNA, 1263 nt, unidentified
  source 1..1263: mol_type = rRNA; note = DP100 16S rRNA sequence
tttgagagtttgatcctggctcaggacgaacgctggcggcgtgcctaatacatgcaagtcgaacgaactctggtattgattggtgcttgcatcatgatttacatttgagtgagtggcgaactggtgagtaacacgtgggaaacctgcccagaagcgggggataacacctggaaacagatgctaataccgcataacaacttggaccgcatggtccgagcttgaaagatggcttcggctatcacttttggatggtcccgcggcgtattagctagatggtggggtaacggctcaccatggcaatgatacgtagccgacctgagagggtaatcggccacattgggactgagacacggcccaaactcctacgggaggcagcagtagggaatcttccacaatggacgaaagtctgatggagcaacgccgcgtgagtgaagaagggtttcggctcgtaaaactctgttgttaaagaagaacatatctgagagtaactgttcaggtattgacggtatttaaccagaaagccacggctaactacgtgccagcagccgcggtaatacgtaggtggcaagcgttgtccggatttattgggcgtaaagcgagcgcaggcggttttttaagtctgatgtgaaagccttcggctcaaccgaagaagtgcatcggaaactgggaaacttgagtgcagaagaggacagtggaactccatgtgtagcggtgaaatgcgtagatatatggaagaacaccagtggcgaaggcggctgtctggtctgtaactgacgctgaggctcgaaagtatgggtagcaaacaggattagataccctggtagtccataccgtaaacgatgaatgctaagtgttggagggtttccgcccttcagtgctgcagctaacgcattaagcattccgcctggggagtacggccgcaaggctgaaactcaaaggaattgacgggggcccgcacaagcggtggagcatgtggtttaattcgaagctacgcgaagaaccttaccaggtcttgacatactatgcaaatctaagagattagacgttcccttcggggacatggatacaggtggtgcatggttgtcgtcagctcgtgtcgtgagatgttgggttaagtcccgcaacgagcgcaacccttattatcagttgccagcattaagttgggcactctggtgagactgccggtgacaaaccggaggaaggtggggatgacgtcaaatcatcatgccccttatgacctgggctacacacgtgctacaatgg

SEQ ID NO: 164 - RNA, 1457 nt, unidentified
  source 1..1457: mol_type = rRNA; note = DP101 16S rRNA sequence
atgagagtttgatcttggctcaggatgaacgctggcggcgtgcctaatacatgcaagtcgaacgaacttccgttaattgattatgacgtacttgtactgattgagattttaacacgaagtgagtggcgaacgggtgagtaacacgtgggtaacctgcccagaagtaggggataacacctggaaacagatgctaataccgtataacagagaaaaccgcatggttttcttttaaaagatggctctgctatcacttctggatggacccgcggcgtattagctagttggtgaggcaaaggctcaccaaggcagtgatacgtagccgacctgagagggtaatcggccacattgggactgagacacggcccagactcctacgggaggcagcagtagggaatcttccacaatggacgcaagtctgatggagcaacgccgcgtgagtgaagaagggtttcggctcgtaaagctctgttgttaaagaagaacgtgggtaagagtaactgtttacccagtgacggtatttaaccagaaagccacggctaactacgtgccagcagccgcggtaatacgtaggtggcaagcgttatccggatttattgggcgtaaagcgagcgcaggcggtcttttaagtctaatgtgaaagccttcggctcaaccgaagaagtgcattggaaactgggagacttgagtgcagaagaggacagtggaactccatgtgtagcggtgaaatgcgtagatatatggaagaacaccagtggcgaaggcggctgtctggtctgcaactgacgctgaggctcgaaagcatgggtagcgaacaggattagataccctggtagtccatgccgtaaacgatgattactaagtgttggagggtttccgcccttcagtgctgcagctaacgcattaagtaatccgcctggggagtacgaccgcaaggttgaaactcaaaagaattgacgggggcccgcacaagcggtggagcatgtggtttaattcgaagctacgcgaagaaccttaccaggtcttgacatcttctgacagtctaagagattagaggttcccttcggggacagaatgacaggtggtgcatggttgtcgtcagctcgtgtcgtgagatgttgggttaagtcccgcaacgagcgcaacccttattactagttgccagcattaagttgggcactctagtgagactgccggtgacaaaccggaggaaggtggggacgacgtcaaatcatcatgccccttatgacctgggctacacacgtgctacaatggatggtacaacgagtcgcgagaccgcgaggttaagctaatctcttaaaaccattctcagttcggactgtaggctgcaactcgcctacacgaagtcggaatcgctagtaatcgcggatcagcatgccgcggtgaatacgttcccgggccttgtacacaccgcccgtcacaccatgagagtttgtaac

SEQ ID NO: 165 - DNA, 1415 bp, unidentified
  source 1..1415: mol_type = genomic DNA; note = DP101 ITS sequence
tccgtaggtgaacctgcggaaggatcattactgtgatttagtactacactgcgtgagcggaacgaaaacaacaacacctaaaatgtggaatatagcatatagtcgacaagagaaatctacgaaaaacaaacaaaactttcaacaacggatctcttggttctcgcatcgatgaagagcgcagcgaaatgcgatacctagtgtgaattgcagccatcgtgaatcatcgagttcttgaacgcacattgcgcccctcggcattccggggggcatgcctgtttgagcgtcgtttccatcttgcgcgtgcgcagagttgggggagcggagcggacgacgtgtaaagagcgtcggagctgcgactcgcctgaaagggagcgaagctggccgagcgaactagactttttttcagggacgcttggcggccgagagcgagtgttgcgagacaacaaaaagctcgacctcaaatcaggtaggaatacccgctgaacttaagcatatcaataagcggaggaaaagaaaccaacagggattgcctcagtagcggcgagtgaagcggcaagagctcagatttgaaatcgtgctttgcggcacgagttgtagattgcaggttggagtctgtgtggaaggcggtgtccaagtcccttggaacagggcgcccaggagggtgagagccccgtgggatgccggcggaagcagtgaggcccttctgacgagtcgagttgtttgggaatgcagctccaagcgggtggtaaattccatctaaggctaaatactggcgagagaccgatagcgaacaagtactgtgaaggaaagatgaaaagcactttgaaaagagagtgaaacagcacgtgaaattgttgaaagggaagggtattgcgcccgacatggggattgcgcaccgctgcctctcgtgggcggcgctctgggctttccctgggccagcatcggttcttgctgcaggagaaggggttctggaacgtggctcttcggagtgttatagccagggccagatgctgcgtgcggggaccgaggactgcggccgtgtaggtcacggatgctggcagaacggcgcaacaccgcccgtcttgaaacatggaccaaggagtctaacgtctatgcgagtgtttgggtgtgaaacccgtacgcgtaatgaaagtgaacgtaggtcggaccccctgccctcggggaggggagcacgatcgaccgatcccgatgtttatcggaaggatttgagtaggagcatagctgttgggacccgaaagatggtgaactatgcctgaatagggtgaagccagaggaaactctggtggaggctcgtagcggttctgacgtgcaaatcgatcgtcgaatttgggtataggggcgaaagactaatcgaaccatctagtagctggttcctgccgaagtttccctcagga

(19) United States
(12) Patent Application Publication
(10) Pub. No.: US 2025/0009814 A1
(43) Pub. Date: Jan. 9, 2025

(54) METHODS AND COMPOSITIONS FOR TREATING MUSCULOSKELETAL DISEASES

(71) Applicant: Solarea Bio, Inc., Cambridge, MA (US)

(72) Inventors: Gerardo V. Toledo, Hopkinton, MA (US); Maria Juliana Soto-Giron, Cambridge, MA (US); Jinwoo Kim, Acton, MA (US); Julie E. Button, Lincoln, MA (US); Eric Michael Schott, West Roxbury, MA (US)

(21) Appl. No.: 18/744,111

(22) Filed: Jun. 14, 2024

Related U.S. Application Data
(63) Continuation of application No. 17/816,371, filed on Jul. 29, 2022, now Pat. No. 12,048,722.
Application No. 17/816,371 is a division of application No. 16/694,876, filed on Nov. 25, 2019, now Pat. No. 11,819,524.
Application No. 16/694,876 is a continuation of application No. PCT/US2019/049823, filed on Sep. 5, 2019.
(60) Provisional application No. 62/863,722, filed on Jun. 19, 2019.
(60) Provisional application No. 62/728,018, filed on Sep. 6, 2018.
(60) Provisional application No. 62/728,019, filed on Sep. 6, 2018.
(60) Provisional application No. 62/728,020, filed on Sep. 6, 2018.
(60) Provisional application No. 62/727,503, filed on Sep. 5, 2018.

(51) Int. Cl. A61K 35/741; A23L 33/125; A23L 33/135; A23L 33/14; A61K 31/702; A61K 35/744; A61K 36/062; A61K 45/06; A61P 19/08; A61P 19/10

(52) U.S. Cl. CPC A61K 35/741; A23L 33/125; A23L 33/135; A23L 33/14; A61K 31/702; A61K 35/744; A61K 36/062; A61K 45/06; A61P 19/08; A61P 19/10; A23V 2002/00; A23V 2400/173; A23V 2400/321; A23V 2400/427

(57) ABSTRACT

Described herein are methods and compositions for using microbial agents (probiotics) and agents that promote growth of certain microbes (prebiotics) for management (including prevention and treatment) of musculoskeletal disorders, including osteoporosis, osteopenia, Paget's disease, stunting, osteoarthritis, osteomyelitis, and delayed or non-union fractures.

### CROSS REFERENCE TO RELATED APPLICATIONS

This application is a continuation of U.S. application Ser. No. 17/816,371 filed Jul. 29, 2022, which application is a divisional of U.S. application Ser. No. 16/694,876 filed Nov. 25, 2019, which application is a continuation of International Application PCT/US2019/049823, filed Sep. 5, 2019, which application claims the benefit of U.S. Provisional Application No. 62/727,503 filed Sep. 5, 2018; U.S. Provisional Application No. 62/728,018, filed Sep. 6, 2018; 62/728,019, filed Sep. 6, 2018; U.S. Provisional Application No. 62/728,020, filed Sep. 6, 2018, and U.S. Provisional Application No. 62/863,722, filed Jun. 19, 2019 all of which are hereby incorporated by reference in their entireties for all purposes.

### SEQUENCE LISTING

The instant application contains a Sequence Listing which has been submitted electronically and is hereby incorporated herein by reference in its entirety. Said XML copy, created on Mar. 23, 2023, is named SBI-001D1_SL.xml, and is 439,687 bytes in size.

### BACKGROUND

The disclosure relates to methods and compositions for treating or preventing musculoskeletal diseases, including osteoporosis, osteopenia, osteoarthritis, suboptimal fracture healing, and osteomyelitis.

Daily consumption of fresh fruits, vegetables, seeds and other plant-derived ingredients of salads and juices is recognized as part of a healthy diet and associated with weight loss, weight management and overall healthy life styles. This is demonstrated clinically and epidemiologically in the “China Study” (Campbell, T. C. and Campbell T. M. 2006. The China Study: startling implications for diet, weight loss and long-term health. Benbella books pp 419) where a lower incidence of inflammatory-related indications were observed in rural areas where diets are whole food plant-based. The benefit from these is thought to be derived from the vitamins, fiber, antioxidants and other molecules that are thought to benefit the microbial flora through the production of prebiotics. These can be in the form of fermentation products from the breakdown of complex carbohydrates and other plant-based polymers. There has been no clear mechanistic association between microbes in whole food plant-based diets and the benefits conferred by such a diet. The role of these microbes as probiotics, capable of contributing to gut colonization and thereby influencing a subject's microbiota composition in response to a plant-based diet, has been underappreciated.

Musculoskeletal disorders, including osteoporosis, osteopenia, Paget's disease, stunting, osteoarthritis, osteomyelitis, delayed or non-union fractures, are potentially disabling conditions whose current treatments are often accompanied by potentially serious negative side effects. Often therapies treat symptoms, while leaving underlying causes, such as chronic inflammation, unaddressed. Therefore, treatments with reduced side-effects and increased efficacy towards alleviating underlying causes represent a long-felt unmet need.

### SUMMARY OF THE INVENTION

Provided for herein is a method of reducing bone loss, comprising administering to a subject in need thereof a pharmaceutical composition comprising an effective amount of at least two heterologous microbes selected from at least two groups selected from proteobacteria, lactic acid bacteria, and yeast, formulated for oral administration.

In some aspects, at least two of the heterologous microbes have at least 97% similarity to Seq ID Nos: 1-66 at the at 16S rRNA or fungal ITS sequence. In some aspects, at least two of the heterologous microbes have at least 98% similarity to Seq ID Nos: 1-66 at the at 16S rRNA or fungal ITS sequence. In some aspects, at least two of the heterologous microbes have at least 98.5% similarity to Seq ID Nos: 1-66 at the at 16S rRNA or fungal ITS sequence. In some aspects, at least two of the heterologous microbes have at least 99% similarity to Seq ID Nos: 1-66 at the at 16S rRNA or fungal ITS sequence. In some aspects, at least two of the heterologous microbes have 100% similarity to Seq ID Nos: 1-66 at the at 16S rRNA or fungal ITS sequence.

In some aspects, the pharmaceutical composition comprises an effective amount of at least three each heterologous microbes, selected from at least two groups selected from proteobacteria, lactic acid bacteria, and yeast, formulated for oral administration.

In some aspects, the pharmaceutical composition further comprises at least one additional microbe from table 4 or table 7. In some aspects, the pharmaceutical composition further comprises a cryoprotectant. In some aspects, the cryoprotectant extends room temperature survival of at least one microbe.

In some aspects, the pharmaceutical composition further comprises a prebiotic.

Also provided for herein is a method of reducing bone loss, comprising administering to a subject in need thereof a medical food composition comprising an effective amount of at least two heterologous microbes selected from at least two groups selected from proteobacteria, lactic acid bacteria, and yeast, formulated for oral administration. In some aspects, at least two of the heterologous microbes have at least 97% similarity to Seq ID Nos: 1-66 at the at 16S rRNA or fungal ITS sequence. In some aspects, the medical food composition comprises an effective amount of at least three of each heterologous microbes selected from at least two groups selected from proteobacteria, lactic acid bacteria, and yeast, formulated for oral administration. In some aspects, the medical food composition further comprises at least one additional microbe from table 4 or table 7. In some aspects, the medical food composition further comprises a cryoprotectant. In some aspects, the cryoprotectant extends room temperature survival of at least one microbe. In some aspects, the medical food composition further comprises a prebiotic.

Also provided for herein is a probiotic composition comprising an effective amount of at least two heterologous microbes selected from at least two groups selected from proteobacteria, lactic acid bacteria, and yeast, formulated for oral administration. In some aspects, at least two of the heterologous microbes have at least 97% similarity to Seq ID Nos: 1-66 at the at 16S rRNA or fungal ITS sequence. In some aspects, the probiotic composition comprises an effective amount of at least three of each heterologous microbes selected from at least two groups selected from proteobacteria, lactic acid bacteria, and yeast, formulated for oral administration. In some aspects, the probiotic composition further comprises at least one additional microbe from table 4 or table 7. In some aspects, the probiotic composition further comprises a cryoprotectant. In some aspects, the cryoprotectant extends room temperature survival of at least one microbe. In some aspects, the medical food composition further comprises a prebiotic.

Also provided for herein is a method of treating osteoarthritis, comprising administering to a subject in need thereof a pharmaceutical composition comprising an effective amount of at least two heterologous microbes selected from at least two groups selected from proteobacteria, lactic acid bacteria, and yeast, formulated for oral administration. In some aspects, at least two of the heterologous microbes have at least 97% similarity to Seq ID Nos: 1-66 at the at 16S rRNA or fungal ITS sequence. In some aspects, the pharmaceutical composition comprises an effective amount of at least three each heterologous microbes, selected from at least two groups selected from proteobacteria, lactic acid bacteria, and yeast, formulated for oral administration. In some aspects, the pharmaceutical composition further comprises at least one additional microbe from table 4 or table 7. In some aspects, the pharmaceutical composition further comprises a cryoprotectant.

Also provided for herein is a method of treating osteomyelitis, comprising administering to a subject in need thereof a pharmaceutical composition comprising an effective amount of at least two heterologous microbes selected from at least two groups selected from proteobacteria, lactic acid bacteria, and yeast, formulated for oral administration. In some aspects, at least two of the heterologous microbes have 97% similarity to Seq ID Nos: 1-66 at the at 16S rRNA or fungal ITS sequence. In some aspects, the pharmaceutical composition comprises an effective amount of at least three each heterologous microbes, selected from at least two groups selected from proteobacteria, lactic acid bacteria, and yeast, formulated for oral administration. In some aspects, the pharmaceutical composition further comprises at least one additional microbe from table 4 or table 7. In some aspects, the pharmaceutical composition further comprises a cryoprotectant.

Also provided for herein is a method of improving healing of non-union or delayed union fractures, comprising administering to a subject in need thereof a pharmaceutical composition comprising an effective amount of at least two heterologous microbes selected from at least two groups selected from proteobacteria, lactic acid bacteria, and yeast, formulated for oral administration. In some aspects, at least two of the heterologous microbes have at least 97% similarity to Seq ID Nos: 1-66 at the at 16S rRNA or fungal ITS sequence. In some aspects, the pharmaceutical composition comprises an effective amount of at least three each heterologous microbes, selected from at least two groups selected from proteobacteria, lactic acid bacteria, and yeast, formulated for oral administration. In some aspects, the pharmaceutical composition further comprises at least one additional microbe from table 4 or table 7. In some aspects, the pharmaceutical composition further comprises a cryoprotectant.

Also provided for herein is a pharmaceutical composition comprising an isolated population of bacterial cells comprising three or more strains present in whole food plant-based diets, wherein each strain is capable of modulating production of one or more short chain fatty acids, vitamin K2, and/or flavones such as apigenin, narigenin, hesperidin, nobiletin, tangeretin in the mammalian gut.

Also provided for herein is a synthetic combination comprising a purified bacterial population, wherein said population comprises at least three unique isolates selected from the group consisting of Pseudomonas, Leuconostoc, Acinetobacter, Aeromonas, Curtobacterium, Escherichia, Lactobacillus, Serratia, Streptococcus, and Stenotrophomonas, Leuconostoc, Pediococous, Deboromyces, Pichia, Hanseniaspora, where the purified bacterial population is capable of modulating production of one or more short chain fatty acids, flavones, and/or vitamin K2 in a mammalian gut.

Also provided for herein is a synthetic combination comprising a purified bacterial population, wherein said population comprises at least 3 isolates from Table 4 or Table 7 where the at least 3 isolates are capable of modulating production of one or more short chain fatty acids selected from the group consisting of acetate, butyrate, and propionate; or the enzymes acetolactate synthase I, N-acetylglutamate synthase, acetate kinase, Acetyl-CoA synthetase, acetyl-CoA hydrolase, Glucan 1,4-alpha-glucosidase, Bile acid symporter Acr3; and/or capable of modulating production of flavones and/or vitamin K2 and wherein the isolates are present in an amount effective to adhere to a mammalian mucosal lining, thereby modulating the bone health markers of a mammal treated with the synthetic combination, as compared to a reference mammal

Also provided for herein is a synthetic population that mimics the composition seen in human stool from patients with desirable bone mineral density or other markers of normal bone health.

Also provided for herein is a synthetic microbial consortia comprising a purified bacterial population of lactic acid bacteria and gamma proteobacteria, wherein the synthetic consortia is capable of modulating production of one or more short chain fatty acids selected from the group consisting of acetate, butyrate, and propionate; and/or capable of modulating production of flavones and/or vitamin K2; and wherein the isolates are present in an amount effective to adhere to a mammalian mucosal lining, thereby modulating the bone health markers, such as bone density, of a mammal treated with the synthetic combination, as compared to a reference mammal.

Also provided for herein is a synthetic microbial consortia comprising a purified bacterial population isolated from a first plant-based sample selected from samples 1-21 in Table 3 artificially associated with a purified bacterial population isolated from a second plant-based sample from selected from samples 1-21 in Table 3, wherein the synthetic microbial consortia is capable of modulating the bone density of a mammal treated with the synthetic microbial consortia, as compared to a reference mammal.

Also provided for herein is a synthetic microbial composition that is not completely viable and can act by releasing metabolites that act in the GI tract of a patient reducing symptoms of osteoporosis or osteopenia.

### BRIEF DESCRIPTION OF FIGURES

These and other features, aspects, and advantages of the present invention will become better understood with regard to the following description, and accompanying drawings, where:

FIGS. 1A-L show plots depicting the diversity of microbial species detected in samples taken from 12 plants usually consumed raw by humans.

FIG. 1A shows bacterial diversity observed in a green chard.

FIG. 1B shows bacterial diversity in red cabbage.

FIG. 1C shows bacterial diversity in romaine lettuce.

FIG. 1D shows bacterial diversity in celery sticks.

FIG. 1E shows bacterial diversity observed in butterhead lettuce grown hydroponically.

FIG. 1F shows bacterial diversity in organic baby spinach.

FIG. 1G shows bacterial diversity in green crisp gem lettuce

FIG. 1H shows bacterial diversity in red oak leaf lettuce.

FIG. 1I shows bacterial diversity in green oak leaf lettuce.

FIG. 1J shows bacterial diversity in cherry tomatoes.

FIG. 1K shows bacterial diversity in crisp red gem lettuce.

FIG. 1L shows bacterial diversity in broccoli juice.

FIG. 2A-C show graphs depicting the taxonomic composition of microbial samples taken from Broccoli Heads (FIG. 2A), Blueberries (FIG. 2B), and Pickled Green Olives (FIG. 2C).

FIG. 3A shows taxonomic composition of ginseng. There is a relatively high diversity represented in this sample with members of Pseudomonas, Pantoea, and Stenotrophomonas.

FIG. 3B shows taxonomic composition of blackberries. The most abundant member is Rahnella aquatilis covering 31% of total composition.

FIG. 3C shows taxonomic composition of squash gourd. The sample is dominated by Lactococcus lactis covering 59% of total composition but also Leuconostoc mesenteroides was present at 3.3% of the bacterial population.

FIG. 3D shows taxonomic composition of broccolini. Ralstonia pickettii covers 44% of entire bacterial community.

FIG. 3E shows taxonomic composition of fermented cabbage. It contained Pediococcus pentosaceus as well as dominant gamma protcobacteria.

FIG. 3F shows taxonomic composition of fermented pepper paste. The sample enriched many lactic acid bacteria such as Lactobacillus paracasei, Lactobacillus casei and Lactobacillus plantarum.

FIG. 4 shows fermentative rates by sample microbes alone or as a community under various conditions in silico. Four microbes were tested in silico for their ability to produce (A) Acetate, (B) Propionate, or (C) Butyrate under rich media or oligofructose conditions alone or as an assembled community.

FIG. 5 shows DMA experimental validation for a combination of strains DP3 and DP9 under nutrient replete and plant fiber media showing that the strains show synergy for increased SCFA production only under plant fiber media but not under rich media.

FIG. 6 shows synergistic acetate production for 4 strains tested as singles, pairs or trios. Cells were grown on blueberry extract media for 4 days in a 24 well plate at 300 RPM and 22° C. The pairs in this experiment were run in duplicate. Spent culture broth was extracted with ethyl acetate and analyzed by gas chromatography with a flame ionization detector (GC-FID) and acetate concentrations measured with a standard curve done in sterile media. The strain DP6 does not produce acetate, while strain DP9 produces 448 uM, and when the 2 are grown together the acetate production is 1500 uM and 1457 uM for both duplicate cultures respectively. This indicates the acetate increased by adding strain DP6 to DP9.

FIG. 7 shows a schematic describing a gut simulator experiment. The experiment comprises an in vitro, system that mimics various sections of the gastrointestinal tract. Isolates of interest are incubated in the presence of conditions that mimic particular stresses in the gastro-intestinal tract (such as low pH or bile salts), heat shock, or metformin. After incubation, surviving populations are recovered. Utilizing this system, the impact of various oral anti-diabetic therapies alone or in combination with probiotic cocktails of interest on the microbial ecosystem can be tested.

FIG. 8 shows a fragment recruitment plot sample for the shotgun sequencing on sample 22 (fermented cabbage) comparing to the reference genome of strain DP3 Leuconostoc mesenteroides-like and the 18× coverage indicating the isolated strain was represented in the environmental sample and it was largely genetically homogeneous.

FIG. 9 shows a schematic detailing the experimental procedure for a pre-clinical model testing the disclosed invention. The experimental design is as follows: Candidate DMAs were evaluated for their therapeutic efficacy in an ovariectomized (OVX) mouse model of postmenopausal osteoporosis. All mice were group housed with 5 mice per cage in individually ventilated cages (IVCs) specifically designed for germ free husbandry [59, 60]. At 12-weeks of age, mice were weighed, had baseline feces collected, and underwent OVX (N=20) or sham (N=10) surgery to deplete estrogen levels and commence the bone resorption process as previously described [61]. 1-day post-surgery, mice were randomly divided into experimental groups and mice began a daily oral gavage regimen (200 uL) of saline (negative control), or SBD111 and continued for 6-weeks. Fecal samples were collected every 3 weeks to monitor the composition of the gut microbiome over time. On the last day of the study, mice received a DXA scan to evaluate systemic BMD, followed by euthanasia and collection of uterine weights, scrum, cecal material, lumbar spine and femurs for downstream analysis.

FIG. 10. Shows ovary weights taken from ovariectomized and sham-treated mice. Ovariectomized (OVX) mice were treated with either water (OVX) or DMA SBD111 for six-weeks post-surgery. At sacrifice, the uterus from each animal was removed and weighed. Uterus measurements were also taken from sham-treated mice. Decreased uterus weight in OVX and SBD111 treated animals indicates successful ovariectomy. Significant differences between groups in A were identified via 1-way ANOVA with a Tukey multiple comparison post-test (*P<0.05, **P<0.01 ***P<0.001, ****P<0.0001)

FIG. 11. Ovariectomized (OVX) mice were treated with either water (OVX) or SBD111 for six-weeks post-surgery. Mice received DXA scans before surgery and six-weeks post-surgery to determine the percent change in bone mineral density (BMD). DXA scans reveal a significant protection against OVX-induced bone loss at the lumbar spine (A) and distal femur (B) in mice treated with SBD111. Significant differences between groups in A and B were identified via 1-way ANOVA with a Tukey multiple comparison post-test (*P<0.05, **P<0.01 ***P<0.001, ****P<0.0001)

FIG. 12. Ovariectomized (OVX) mice were treated with either water (OVX) or SBD111 for six-weeks post-surgery. At sacrifice, lumbar spine (L1-L4) were removed and analyzed by micro computed tomography (MicroCT) for trabecular bone volume (BV/TV) (A) and trabecular thickness (B). MicroCT scans reveal a significant protection against OVX-induced bone loss at the lumbar spine as indicated by BV/TV (A) and Trabecular Thickness (B) in mice treated with SBD111. Significant differences between groups in A and B were identified via 1-way ANOVA with a Tukey multiple comparison post-test (*P<0.05, **P<0.01 ***P<0.001, ****P<0.0001)

FIG. 13. C57bl/6J mice were placed on a high fat diet (60% kcal fat) for 12 weeks to induce obesity. Mice were then treated with either water control (obese) or SBD102 (obese+SBD102), a DMA consisting of prebiotic plant fibers and probiotic microbes, for 8-weeks. Bone mineral density (BMD) was measured by whole body dual x-ray absorptiometry (DXA) (A), and trabecular bone volume was measured at the distal femur by micro computed tomography (MicroCT) (B). Whole body DXA revealed an 8.5% decrease in BMD in obese mice compared to lean, that was prevented by treatment with SBD102 (A). A similar effect was observed by microCT of the distal femur, were obese mice had lower trabecular bone volume compared to lean mice that was prevented by treatment with SBD102 (B). Representative images of the microCT are depicted in (C).

FIG. 14. Shows the composition of the gut microbial community of the sham and OVX mice at the baseline and six-weeks post-surgery time points with SBD111. Overall, Bacteroides thetaiotaomicron was the most prevalent taxon detected among the mice groups encompassing more than 50% of the total community on average, followed by Lactobacillus johnsonii with abundance values between 8.8% and 24.2%, excepting the sham baseline group where Akkermansia municiphila was the second most abundant taxon (21.3% on average). In the case of the SBD111 group, Bifidobacterium pseudolongum showed an increase in abundance at week 6 (from 5% to 7.8% of the total community).

FIG. 15. Fragment recruitment plots showing fragment recruitment of the Bifidobacterium pseudolongum reference genome in the gut metagenomes from mice treated withOVX and SBD111 at the baseline and six-weeks post-surgery. Recruitment plots were built using scripts available at the enveomics toolbox (Rodriguez-R and Konstantinidis 2016). Table 13 shows the individuals changes in B. pseudolongum as seem by coverage.

FIG. 16. Fragment recruitment plots showing fragment recruitment of the Bifidobacterium pseudolongum and Lactobacillus johnsonii reference genomes in the gut metagenomes from mice treated with SBD111 taken at the week 6 time point. These show that the microbes were present at week 6 post-surgery. The recruitment plots were built using scripts available at the enveomics toolbox (Rodriguez-R and Konstantinidis 2016).

FIG. 17 Fragment recruitment of the genomes from the microbes used in SDB111 in the gut metagenomes from mice treated with SBD111 at the baseline and week 6 time points. Recruitment plots were built using scripts available at the enveomics toolbox (Rodriguez-R and Konstantinidis 2016).

FIG. 18A Metabolic pathways significantly different between baseline and 6 weeks of treatment in OVX mice (Tukey-Kramer post-hoc test, P<0.05).

FIG. 18B Metabolic pathways significantly different between baseline and 6 weeks of treatment in SBD111-treated OVX mice (Tukey-Kramer post-hoc test, P<0.05).

FIG. 18C Metabolic pathways significantly different between untreated OVX mice (OVX) and mice given a sham surgery (sham) after 6 weeks of treatment (Tukey-Kramer post-hoc test, P<0.05).

FIG. 18D Metabolic pathways significantly different between untreated OVX (OVX) mice and mice treated with SBD111 after 6 weeks of treatment (Tukey-Kramer post-hoc test, P<0.05).

FIG. 19A Comparison of relative abundance of genes related to L-rhamnose degradation between OVX group and SBD111-treated group. Differences between groups were assessed by Mann-Whitney U test (*P<0.05, **P<0.01). rhaD, rhamnulose-1-phosphage aldolase; rhaB, rhamnulokinase; rhaA, L-rhamnose isomerase; rhaM, L-rhamnose mutarotase.

FIG. 19B Comparison of relative abundance of genes related to SCFA production between OVX group and SBD111-treated group. Differences between groups were assessed by Mann-Whitney U test (*P<0.05, **P<0.01). Butyrate kinase (buk) and phosphotransbutyrylase (ptb) were selected as marker genes representing butyrate production. Pyruvate dehydrogenase (pdh), phosphate acetyltransferase (pta) and acetate kinase (ackA) represent acetate production. L-lactate dehydrogenase (ldh) are involved in lactate production pathway.

FIG. 19C Comparison of relative abundance of genes related to glycoside hydrolase between OVX group and SBD111-treated group. Differences between groups were assessed by Mann-Whitney U test (*P<0.05, **P<0.01). GH15, glucoamylase; GH18, chitinase; GH23, peptidoglycan lyase; GH32, invertase; GH43, β-xylosidase; GH73, lysozyme; GH88, unsaturated glucuronyl hydrolases; GH95, α-L-fucosidase; GH109, α-N-acetylgalactosaminidase.

FIG. 19D Comparison of relative abundance of genes related to vitamin K2 biosynthesis between OVX group and SBD111-treated group. Differences between groups were assessed by Mann-Whitney U test (*P<0.05, **P<0.01). MenA, 1,4-dihydroxy-2-naphthoate prenyltransferase; MenD, 2-succinyl-5-enolpyruvyl-6-hydroxy-3-cyclohexene-1-carboxylate synthase, and MenG, demethylmenaquinone methyltransferase.

FIG. 19E Comparison of relative abundance of alkaline phosphate gene between OVX group and SBD111-treated group at 6 weeks. Differences between groups were assessed by Mann-Whitney U test (*P<0.05, **P<0.01).

FIG. 20 shows viability at different timepoints after cryopreservation using PBS, DMSO, or Cryobuffer solutions to store bacteria.

### DETAILED DESCRIPTION

Musculoskeletal disorders, including osteoporosis and osteopenia, represent a medical challenge presently without a satisfactory remedy. Approximately 10 million Americans over the age of 50 are currently living with osteoporosis or osteopenia culminating in 1.5 million fractures annually. The high incidence of disease leads to an annual economic burden of $17 billion that couples with significantly reduced quality of life. The current standards of care including anti-resorptive and anabolic therapies are limited due to their side effects and restrictive costs, leading to the current unmet need for a safe, effective, and low cost therapeutic that prevents bone loss.

Osteoarthritis (OA), another musculoskeletal disorder, is one of most prevalent diseases in the world, afflicting 31 million individuals in the US, and projected to impact 45 million by 2030. The United States reports the highest incidence of OA with 13% of US adult population affected, and more than 80% of persons over the age of 75 having some degree of disease. OA is a degenerative disease with multiple origins, characterized by progressive cartilage erosion, joint effusion, synovial hyperplasia, subchondral bone sclerosis, and osteophyte formation. Three main types of osteoarthritis are typically identified: aging-related, obesity-related, and post-traumatic. All of these varieties, however, share a root cause of deleterious inflammation.

Affected patients are left in a perpetual state of pain and discomfort, relying on non-steroidal anti-inflammatory drugs (NSAIDs) and opioid pain killers for relief, until end stage disease requires a total joint replacement to restore functionality to the ailing joint. A need for a therapy that modulates systemic inflammation to reduce or reverse the symptoms of osteoarthritis with naturally occurring products is needed for patients to avoid the side effects of current therapies.

Another musculoskeletal disorder involves fractures that do not properly heal. Fractures are a common orthopedic problem, with over 2 million occurring per year in the U.S. With treatment, most broken bones will heal over a 6 to 8-week period without clinically relevant delay. Delayed union and nonunion, the failure of a fractured bone to heal, occurs in approximately 5-10% of all fractures. Moreover, delayed and nonunion is associated with significant morbidity. (Amin et al. 2014)

Importantly, multiple clinical studies have demonstrated that obesity/type 2 diabetes (T2D) are risk factors for fracture nonunion. This is supported by previous studies demonstrating that mice fed a high-fat diet to induce obesity/T2D have impaired fracture healing. Despite this, little is known about the mechanism(s) that increase the risk of nonunion in obese patients, and there are no accepted therapeutic approaches to address the delay in healing that obese/T2D patients experience. (Zura et al. 2016) Thus, strategies to mitigate the deleterious effect of obesity/T2D on fracture are a critical unmet need.

One additional indication is osteomyelitis, which is inflammation of the bone or bone marrow. Although sometimes caused by infection, treatment of osteomyelitis could by aided by administration of probiotic compositions described herein. Modulation of the host immune system by intentionally dosed microbes could mitigate damage done by an overactive immune system or decrease recovery time by improving targeting of the immune system.

### Advantages and Utility

Briefly, and as described in more detail below, described herein are methods and compositions for using microbial agents (probiotics) and agents that promote growth of certain microbes (prebiotics) for management (including prevention and treatment) of musculoskeletal disorders, including osteoporosis, osteopenia, Paget's disease, stunting, osteoarthritis, osteomyelitis, and delayed or non-union fractures.

Several features of the current approach should be noted. It is based on development of synergistic combinations of microbes as on those found in fruits and vegetables consumed as part of a plant-based diet. The combinations are based, in part, on analyses of biochemical pathways catalyzed by genes in these microbes and selection of microbial combinations that promote beneficial metabolic changes in a subject through the biochemical reactions they catalyze such as the production of short chain fatty acids (SCFA).

Advantages of this approach are numerous. They include reduction of the morbidity associated with musculoskeletal disorders, such as osteoporosis or osteopenia, without the use of traditional drugs and the side effects they can sometimes cause. The invention can also reduce chronic inflammation.

The invention is useful for providing health benefits associated with consumption of a plant-based diet, as the diet microbes and fibers are delivered in concentrated form. This can reduce the burden on a subject to ingest potentially unreasonable or inconvenient amounts of particular plants and/or plant-based products, such as fermented foods.

### Definitions

Terms used in the claims and specification are defined as set forth below unless otherwise specified.

The term “ameliorating” refers to any therapeutically beneficial result in the treatment of a disease state, e.g., a metabolic disease state, including prophylaxis, lessening in the severity or progression, remission, or cure thereof.

The term “in situ” refers to processes that occur in a living cell growing separate from a living organism, e.g., growing in tissue culture.

The term “in vivo” refers to processes that occur in a living organism.

The term “mammal” as used herein includes both humans and non-humans and include but is not limited to humans, non-human primates, canines, felines, murines, bovines, equines, and porcines.

As used herein, the term “derived from” includes microbes immediately taken from an environmental sample and also microbes isolated from an environmental source and subsequently grown in pure culture. The term “derived from” also includes material isolated from the recited source, and materials obtained using the isolated materials (e.g., cultures of microorganisms made from microorganisms isolated from the recited source).

The term percent “identity,” in the context of two or more nucleic acid or polypeptide sequences, refer to two or more sequences or subsequences that have a specified percentage of nucleotides or amino acid residues that are the same, when compared and aligned for maximum correspondence, as measured using one of the sequence comparison algorithms described below (e.g., BLASTP and BLASTN or other algorithms available to persons of skill) or by visual inspection. Depending on the application, the percent “identity” can exist over a region of the sequence being compared, e.g., over a functional domain, or, alternatively, exist over the full length of the two sequences to be compared.

For sequence comparison, typically one sequence acts as a reference sequence to which test sequences are compared. When using a sequence comparison algorithm, test and reference sequences are input into a computer, subsequence coordinates are designated, if necessary, and sequence algorithm program parameters are designated. The sequence comparison algorithm then calculates the percent sequence identity for the test sequence(s) relative to the reference sequence, based on the designated program parameters.

Optimal alignment of sequences for comparison can be conducted, e.g., by the local homology algorithm of Smith & Waterman, Adv. Appl. Math. 2:482 (1981), by the homology alignment algorithm of Needleman & Wunsch, J. Mol. Biol. 48:443 (1970), by the search for similarity method of Pearson & Lipman, Proc. Nat'l. Acad. Sci. USA 85:2444 (1988), by computerized implementations of these algorithms (GAP, BESTFIT, FASTA, and TFASTA in the Wisconsin Genetics Software Package, Genetics Computer Group, 575 Science Dr., Madison, Wis.), or by visual inspection (see generally Ausubel et al., infra).

One example of an algorithm that is suitable for determining percent sequence identity and sequence similarity is the BLAST algorithm, which is described in Altschul et al., J. Mol. Biol. 215:403-410 (1990). Software for performing BLAST analyses is publicly available through the National Center for Biotechnology Information (www.ncbi.nlm.nih.gov/).

In some cases, alignment of an entire sequence is not necessary for identification or comparison purposes regarding a microbial entity. In such a case, a so-called diagnostic subsequence can be used. The term “diagnostic subsequence” refers to a portion of a known sequence which would be identified and used by one of skill in the art to identify or compare two or more microbial entities. One, non-limiting example is utilization of subsequences of 16S rRNA sequences found in Asgari et al (2018, bioRxiv).

The term “effective amount” is an amount that is effective to ameliorate a symptom of a disease. An effective amount can also be an amount effective for prophylaxis of a particular disease. More generally, an effective amount is an amount sufficient to produce a desired effect, e.g., an amount effective for alteration of the microbial content of a subject's microbiota.

The term “defined microbial assemblage” or “DMA” refers to a combination of two or more microbial strains (bacterial or fungal) wherein the two or more microbial strains are chosen because they are predicted to achieve a particular synergistic result when applied in concert. DMA compositions preferably further comprise prebiotics or other fiber sources predicted to heighten the desired effect of the microbial strains applied. A DMA is rationally designed to achieve a particular benefit, such as increase SCFA production in the gut lumen.

The term “SBD” refers to a DMA when it is used as a therapeutic intervention in a preclinical or clinical study.

As used herein the term “method” refers to manners, means, techniques and procedures for accomplishing a given task including, but not limited to, those manners, means, techniques and procedures either known to, or readily developed from known manners, means, techniques and procedures by practitioners of the chemical, pharmacological, biological, biochemical and medical arts.

As used herein, the term “treating” includes abrogating, substantially inhibiting, slowing or reversing the progression of a condition, substantially ameliorating clinical or aesthetical symptoms of a condition.

As used herein, the term “preventing” includes completely or substantially reducing the likelihood or occurrence or the severity of initial clinical or aesthetical symptoms of a condition.

As used herein, the term “about” includes variation of up to approximately +/−10% and that allows for functional equivalence in the product.

As used herein, the term “colony-forming unit” or “cfu” is an individual cell that is able to clone itself into an entire colony of identical cells.

As used herein all percentages are weight percent unless otherwise indicated.

As used herein, “viable organisms” are organisms that are capable of growth and multiplication. In some embodiments, viability can be assessed by numbers of colony-forming units that can be cultured. In some embodiments viability can be assessed by other means, such as quantitative polymerase chain reaction.

“Microbiota” refers to the community of microorganisms that occur (sustainably or transiently) in and on a plant or an animal subject, typically a mammal such as a human, including eukaryotes, archaea, bacteria, and viruses (including bacterial viruses i.e., phage).

“Microbiome” refers to the genetic content of the communities of microbes that live inside and on the human body, or inside or outside a plant, both sustainably and transiently, including eukaryotes, archaca, bacteria, and viruses (including bacterial viruses (i.e., phage)), wherein “genetic content” includes genomic DNA, RNA such as ribosomal RNA, the epigenome, plasmids, and all other types of genetic information.

The term “subject” refers to any animal subject including humans, laboratory animals (e.g., primates, rats, mice), livestock (e.g., cows, sheep, goats, pigs, turkeys, and chickens), and household pets (e.g., dogs, cats, and rodents). The subject may be suffering from a dysbiosis, including, but not limited to, an infection due to a gastrointestinal pathogen or may be at risk of developing or transmitting to others an infection due to a gastrointestinal pathogen.

The “colonization” of a host organism includes the non-transitory residence of a bacterium or other microscopic organism. As used herein, “reducing colonization” of a host subject's gastrointestinal tract (or any other microbiotal niche) by a pathogenic bacterium includes a reduction in the residence time of the pathogen in the gastrointestinal tract as well as a reduction in the number (or concentration) of the pathogen in the gastrointestinal tract or adhered to the luminal surface of the gastrointestinal tract. Measuring reductions of adherent pathogens may be demonstrated, e.g., by a biopsy sample, or reductions may be measured indirectly, e.g., by measuring the pathogenic burden in the stool of a mammalian host.

A “combination” of two or more bacteria includes the physical co-existence of the two bacteria, either in the same material or product or in physically connected products, as well as the temporal co-administration or co-localization of the two bacteria.

As used herein “heterologous” designates organisms to be administered that are not naturally present in the same proportions as in the therapeutic composition as in subjects to be treated with the therapeutic composition. These can be organisms that are not normally present in individuals in need of the composition described herein, or organisms that are not present in sufficient proportion in said individuals. These organisms can comprise a synthetic composition of organisms derived from separate plant sources or can comprise a composition of organisms derived from the same plant source, or a combination thereof.

Compositions disclosed herein can be used to treat osteoporosis or osteopenia. Osteoporosis is a systemic skeletal disease characterized by decreasing bone mass and microarchitectural deterioration of bone tissue that leads to an increased risk for bone fragility and fracture. In patients without fragility fracture, osteoporosis is often diagnosed by low bone mineral density (BMD). The international reference standard for the description of osteoporosis in postmenopausal women and in men is a femoral neck or lumbar spine BMD of 2.5 standard deviations (SD) or more below the young female adult mean. Osteopenia is a less severe form of low BMD, defined by the international standard as between 1 and 2.5 SD below the young female average. As defined herein “osteoporosis or osteopenia” indicates a condition where the subject's bone mass per unit volume is reduced. Osteoporosis indicates bone mass reduction to a level below that required for the adequate mechanical support function of the bone. Osteopenia is a milder disease where bone mass per unit is reduced but not to the extent seen in osteoporosis. Patients with osteopenia may subsequently suffer from osteoporosis.

As used herein, “bone density” indicates “bone mineral density” (BMD).

In some embodiments, compositions disclosed herein can be used to treat osteoarthritis. As used herein, the term “osteoarthritis” (abbreviated as “OA”), refers to the disease also known as osteoarthrosis and degenerative joint disease, characterized by inflammation and damage to, or loss of cartilage in any joint or joints, and joint pain. Clinical standards for diagnosing osteoarthritis in subjects including mammalian subjects such as canines and humans are well known and include for example swelling or enlargement of joints, joint tenderness or pain, decreased range of motion in joints, visible joint deformities such as bony growths, and crepitus. Symptoms can be identified by clinical observation and history, or imaging including MRI and X-ray. Criteria for diagnosing the presence of absence of OA and severity or degree of OA include but are not limited to the ACR Criteria for knee OA (R. Altman et al., Development of criteria for the classification and reporting of osteoarthritis: Classification of osteoarthritis of the knee: Diagnostic and Therapeutic Criteria Committee of the American Rheumatism Association. ARTHRITIS RHEUM. August 29 (8): 1039-1049 (1986), functional status criteria according to WOMAC (N. Bellamy et al., 1988, Validation study of WOMAC: a health status instrument for measuring clinically important patient relevant outcomes to antirheumatic drug therapy in patients with osteoarthritis of the hip or knee. J RHEUMATOL 15:1833-1840), and radiological standards for evaluating OA disease severity according to the Kellgren and Lawrence method for knee OA (Kellgren, J. H. and J. S. Lawrence, Radiological assessment of osteo-arthrosis. ANN RHEUM DIS 16:494-502).

In some embodiments, compositions disclosed herein can be used to improve fracture healing. The term “fracture”, as used herein, refers to a disruption in the integrity of a living bone involving injury to bone marrow, periosteum, and adjacent soft tissues. Many types of fractures exist such as, for example, pathological, stress, non-union, delayed-union, and greenstick fractures. A fracture includes open and closed fractures.

The term “fracture line” refers to the line across where disruption of the integrity of the living bone has occurred.

The term “non-union” fracture refers to the fractures which are not completely healed nine months after the initial fracture. These are commonly found in clavicle fractures that are not healed usually within three months, and are usually painful and require surgical fixation.

The term “delayed-union” refers to a fracture that has not healed at least about six months post injury.

In some embodiments, compositions disclosed herein can be used to prevent or treat osteomyelitis. As used herein, “osteomyelitis” is defined as inflammation of the bone or bone marrow. In some embodiments, osteomyelitis is caused by an infection.

Throughout this application, various embodiments of this invention can be presented in a range format. It should be understood that the description in range format is merely for convenience and brevity and should not be construed as an inflexible limitation on the scope of the invention. Accordingly, the description of a range should be considered to have specifically disclosed all the possible subranges as well as individual numerical values within that range.

It is appreciated that certain features of the invention, which are, for clarity, described in the context of separate embodiments, can also be provided in combination in a single embodiment. Conversely, various features of the invention, which are, for brevity, described in the context of a single embodiment, can also be provided separately or in any suitable subcombination or as suitable in any other described embodiment of the invention. Certain features described in the context of various embodiments are not to be considered essential features of those embodiments, unless the embodiment is inoperative without those elements.

It must be noted that, as used in the specification and the appended claims, the singular forms “a,” “an” and “the” include plural referents unless the context clearly dictates otherwise.

The following abbreviations are used in this specification and/or Figures: ac=acetic acid; but=butyric acid; ppa=propionic acid; ctoh=ethanol; lac_L=lactic acid.

### Methods of the Invention

The administration of the microbial composition can be accomplished orally or rectally, although administration is not limited to these methods. In some embodiments, the microbial composition is administered orally. In some embodiments, the microbial composition is delivered rectally. In some embodiments, the administration of the microbial composition occurs at regular intervals. In some embodiments, the administration occurs daily.

The microbial composition can be administered via typical pharmacological means, such as slurries, capsules, microcapsules, or solutions, although means of administration are not limited to these methods. In some embodiments, an enteric capsule or enteric microcapsule is used. In some embodiments the pharmaceutical composition involving the microbial composition described herein will be fresh or frozen prior to application. In some embodiments, said pharmaceutical composition will be lyophilized or otherwise treated to increase stability or otherwise obtain a benefit from said treatment.

### Compositions of the Invention

In certain embodiments, compositions of the invention comprise probiotic compositions formulated for administration or consumption, with a prebiotic and any necessary or useful excipient. In other embodiments, compositions of the invention comprise probiotic compositions formulated for consumption without a prebiotic. Probiotic compositions of the invention are preferably isolated from foods normally consumed raw and isolated for cultivation. Preferably, microbes are isolated from different foods normally consumed raw, but multiple microbes from the same food source may be used.

It is known to those of skill in the art how to identify microbial strains. Bacterial strains are commonly identified by 16S rRNA gene sequence. Fungal species can be identified by sequence of the internal transcribed space (ITS) regions of rDNA.

One of skill in the art will recognize that the 16S rRNA gene and the ITS region comprise a small portion of the overall genome, and so sequence of the entire genome (whole genome sequence) may also be obtained and compared to known species.

Additionally, multi-locus sequence typing (MLST) is known to those of skill in the art. This method uses the sequences of 7 known bacterial genes, typically 7 housekeeping genes, to identify bacterial species based upon sequence identity of known species as recorded in the publicly available PubMLST database. Housekeeping genes are genes involved in basic cellular functions.

In certain embodiments, bacterial entities of the invention are identified by comparison of the 16S rRNA sequence to those of known bacterial species, as is well understood by those of skill in the art. In certain embodiments, fungal species of the invention are identified based upon comparison of the ITS sequence to those of known species (Schoch et al PNAS 2012). In certain embodiments, microbial strains of the invention are identified by whole genome sequencing and subsequent comparison of the whole genome sequence to a database of known microbial genome sequences. While microbes identified by whole genome sequence comparison, in some embodiments, are described and discussed in terms of their closest defined genetic match, as indicated by 16S rRNA gene sequence, it should be understood that these microbes are not identical to their closest genetic match and are novel microbial entities. This can be shown by examining the Average Nucleotide Identity (ANI) of microbial entities of interest as compared to the reference strain that most closely matches the genome of the microbial entity of interest. ANI is further discussed in example 6.

In other embodiments, microbial entities described herein are functionally equivalent to previously described strains with homology at the 16S rRNA or ITS region. In certain embodiments, functionally equivalent bacterial strains have at least 95% identity at the 16S rRNA region and functionally equivalent fungal strains have at least 95% identity at the ITS region. In certain embodiments, functionally equivalent bacterial strains have at least 96% identity at the 16S rRNA region and functionally equivalent fungal strains have at least 96% identity at the ITS region. In certain embodiments, functionally equivalent bacterial strains have at least 97% identity at the 16S rRNA region and functionally equivalent fungal strains have at least 97% identity at the ITS region. In certain embodiments, functionally equivalent bacterial strains have at least 98% identity at the 16S rRNA region and functionally equivalent fungal strains have at least 98% identity at the ITS region. In certain embodiments, functionally equivalent bacterial strains have at least 99% identity at the 16S rRNA region and functionally equivalent fungal strains have at least 99% identity at the ITS region. In certain embodiments, functionally equivalent bacterial strains have at least 99.5% identity at the 16S rRNA region and functionally equivalent fungal strains have at least 99.5% identity at the ITS region. In certain embodiments, functionally equivalent bacterial strains have 100% identity at the 16S rRNA region and functionally equivalent fungal strains have 100% identity at the ITS region.

16S rRNA sequences for strains tolerant of relevant stressors (described in table 7) are found in SEQ ID NOs 1-63. 16S rRNA is one way to classify bacteria into operational taxonomic units (OTUs). Bacterial strains with 97% sequence identity at the 16S rRNA locus are considered to belong to the same OTU. A similar calculation can be done with fungi using the ITS locus in place of the bacterial 16S rRNA sequence.

In some embodiments, the invention provides a probiotic composition for the treatment of osteoporosis, osteopenia, Paget's disease, or stunting comprising a mixture of Lactic acid bacteria, such as Pediococcus spp, Leuconostoc spp, Lactobacillus spp, Lactobacillus crispatus, Lactobacillus plantarum, Lactobacillus reuteri, combined with non-lactic acid bacteria isolated or identified from samples described in Table 3 or described in Table 4. In some embodiments, the invention provides a fermented probiotic composition for the treatment of bone diseases comprising a mixture of Pediococcus pentosaceus and/or Leuconostoc mesenteroides and at least one non-lactic acid bacterium, preferably a bacterium classified as a gamma proteobacterium or a filamentous fungus or yeast. Some embodiments comprise the probiotic being in a capsule or microcapsule adapted for enteric delivery.

The compositions disclosed herein are derived from edible plants and can comprise a mixture of microorganisms, comprising bacteria, fungi, archaca, and/or other indigenous or exogenous microorganisms, all of which work together to form a microbial ecosystem with a role for each of its members.

In some embodiments, species of interest are isolated from plant-based food sources normally consumed raw. These isolated compositions of microorganisms from individual plant sources can be combined to create a new mixture of organisms. Particular species from individual plant sources can be selected and mixed with other species cultured from other plant sources, which have been similarly isolated and grown. In some embodiments, species of interest are grown in pure cultures before being prepared for consumption or administration. In some embodiments, the organisms grown in pure culture are combined to form a synthetic combination of organisms.

In some embodiments, the microbial composition comprises proteobacteria or gamma proteobacteria. In some embodiments, at least one species from each of 4 groups is present, the four groups being: Lactic Acid bacteria, Bacilli, proteobacteria, and yeast. In some embodiments, at least one microbe from a group other than the four stated above is also present. In some embodiments, the microbial composition comprises several species of Pseudomonas. In some embodiments, species from another genus are also present. In some embodiments, a species from the genus Duganella is also present. In some embodiments of said microbial composition, the population comprises at least three unique isolates selected from the group consisting of Pseudomonas, Acinetobacter, Aeromonas, Curtobacterium, Escherichia, Lactobacillus, Serratia, Streptococcus, and Stenotrophomonas. In some embodiments, the bacteria are selected based upon their ability to degrade fibers, including plant fibers, and to modulate production of one or more branch chain fatty acids, short chain fatty acids, and/or flavones in a mammalian gut.

In some embodiments, microbial compositions comprise isolates that are capable of modulating production or activity of the enzymes involved in fatty acid metabolism, such as acetolactate synthase I, N-acetylglutamate synthase, acetate kinase, Acetyl-CoA synthetase, acetyl-CoA hydrolase, Glucan 1,4-alpha-glucosidase, or Bile acid symporter Acr3.

In some embodiments, the administered microbial compositions colonize the treated mammal's digestive tract. In some embodiments, these colonizing microbes comprise bacterial assemblages present in whole food plant-based diets. In some embodiments, these colonizing microbes comprise Pseudomonas with a diverse species denomination that is present and abundant in whole food plant-based diets. In some embodiments, these colonizing microbes reduce free fatty acids absorbed into the body of a host by absorbing the free fatty acids in the gastrointestinal tract of mammals. In some embodiments, these colonizing microbes comprise genes encoding metabolic functions related to desirable health outcomes such as increased bone mineral density, prevention of loss of bone mineral density, improved bone turnover markers, or improved low-grade inflammatory metabolic indicators, etc.

Some embodiments comprise bacteria that are not completely viable but act by releasing metabolites that act in the gastro-intestinal tract of a patient promoting bone health or other desirable outcome. Some embodiments comprise a prebiotic composition derived from metabolites present in whole food plant-based materials, identified and enriched as part of the formula for oral delivery.

### Prebiotics

Prebiotics, in accordance with the teachings of this invention, comprise compositions that promote the growth of beneficial bacteria in the intestines. Prebiotic substances can be consumed by a relevant probiotic, or otherwise assist in keeping the relevant probiotic alive or stimulate its growth. When consumed in an effective amount, prebiotics also beneficially affect a subject's naturally-occurring gastrointestinal microflora and thereby impart health benefits apart from just nutrition. Prebiotic foods enter the colon and serve as substrate for the endogenous bacteria, thereby indirectly providing the host with energy, metabolic substrates, and essential micronutrients. The body's digestion and absorption of prebiotic foods is dependent upon bacterial metabolic activity, which salvages energy for the host from nutrients that escaped digestion and absorption in the small intestine.

Prebiotics help probiotics flourish in the gastrointestinal tract, and accordingly, their health benefits largely are indirect. Metabolites generated by colonic fermentation by intestinal microflora, such as short-chain fatty acids, can play important functional roles in the health of the host. Prebiotics can be useful agents for enhancing the ability of intestinal microflora to provide benefits to their host.

Prebiotics, in accordance with the embodiments of this invention, include, without limitation, mucopolysaccharides, oligosaccharides, polysaccharides, amino acids, vitamins, nutrient precursors, proteins, and combinations thereof.

According to particular embodiments, compositions comprise a prebiotic comprising a dietary fiber, including, without limitation, polysaccharides and oligosaccharides. These compounds have the ability to increase the number of probiotics, and augment their associated benefits. For example, an increase of beneficial Bifidobacteria likely changes the intestinal pH to support the increase of Bifidobacteria, thereby decreasing pathogenic organisms.

Non-limiting examples of oligosaccharides that are categorized as prebiotics in accordance with particular embodiments include fructooligosaccharides, inulins, isomalto-oligosaccharides, lactilol, lactosucrose, lactulose, pyrodextrins, soy oligosaccharides, transgalacto-oligosaccharides, cellulose, and xylo-oligosaccharides.

According to other particular embodiments, compositions comprise a prebiotic comprising an amino acid.

Prebiotics are found naturally in a variety of foods including, without limitation, cabbage, bananas, berries, asparagus, garlic, wheat, oats, barley (and other whole grains), flaxseed, tomatoes, Jerusalem artichoke, onions and chicory, greens (e.g., dandelion greens, spinach, collard greens, chard, kale, mustard greens, turnip greens), and legumes (e.g., lentils, kidney beans, chickpeas, navy beans, white beans, black beans). Generally, according to particular embodiments, compositions comprise a prebiotic present in a sweetener composition or functional sweetened composition in an amount sufficient to promote health and wellness.

In particular embodiments, prebiotics also can be added to high-potency sweeteners or sweetened compositions. Non-limiting examples of prebiotics that can be used in this manner include fructooligosaccharides, xylooligosaccharides, galactooligosaccharides, and combinations thereof.

Many prebiotics have been discovered from dietary intake including, but not limited to: antimicrobial peptides, polyphenols, Okara (soybean pulp by product from the manufacturing of tofu), polydextrose, lactosucrose, malto-oligosaccharides, gluco-oligosaccharides (GOS), fructo-oligosaccharides (FOS), xantho-oligosaccharides, soluble dietary fiber in general. Types of soluble dietary fiber include, but are not limited to, psyllium, pectin, or inulin. Phytoestrogens (plant-derived isoflavone compounds that have estrogenic effects) have been found to have beneficial growth effects of intestinal microbiota through increasing microbial activity and microbial metabolism by increasing the blood testosterone levels, in humans and farm animals. Phytoestrogen compounds include but are not limited to: Oestradiol, Daidzein, Formononetin, Biochainin A, Genistein, and Equol.

Dosage for the compositions described herein are deemed to be “effective doses,” indicating that the probiotic or prebiotic composition is administered in a sufficient quantity to alter the physiology of a subject in a desired manner. In some embodiments, the desired alterations include reducing osteoporosis or osteopenia and sequelae associated with these conditions. In some embodiments, the desired alterations occur in a post-menopausal subject. Vitamin K2 and osteoporosis:

Vitamin K is found in many fruits and vegetables including broccoli, grapes, lettuce, and olives and plays a role in a wide range of biological activities including calcium metabolism, cell proliferation, oxidative stress, and inflammation. Vitamin K2 (menaquinone) plays a vital role in bone synthesis and is produced by bacteria residing in the gastrointestinal tract. Vitamin K2 affects the proliferation and differentiation of osteoblasts, leading to increased osteoblast activity and bone matrix production. Specifically, Vitamin K2 stimulates the expression of osteoprotegerin (OPG) and inhibits the expression of receptor activator of nuclear factor kappa-B ligand (RANKL) on osteoblasts, leading to increased proliferation and activation. Vitamin K2 has also been shown to inhibit osteoclastic bone resorption, preventing the breakdown of bone.

In some embodiments, the compositions of the invention improve Vitamin K2 absorption. In some embodiments, the compositions of the invention produce Vitamin K2 in the gut of a subject. In some embodiments, the microbes of the invention are selected based upon their having genes involved in biosynthetic pathways for producing Vitamin K2.

In some embodiments, the composition comprises a cryoprotectant. In general, a cryoprotectant functions through work by dissolving in water, lowering the melting point or a composition containing cells, and preventing or limiting intracellular and extracellular crystals from forming in cells during a freezing process. A cryoprotectant can allow for preservation of strain viability for prolonged periods of time, including extending viability for years. In some embodiments, the cryoprotectant is a prebiotic. In some embodiments, the cryoprotectant includes glycerol, trehalose, or Dimethyl sulfoxide (DMSO). In some embodiments, the cryoprotectant is derived from a plant source. In some embodiments, viability, measured at room temperature, is increased for at least one microbe by addition of cryoprotectant to a composition comprising said microbe wherein the composition is stored frozen. In some embodiments, viability is increased by at least 10, 15, 25, 35, 45, 50, 55, 65, 75, 85, 95, or 100 percent. Typically, A cryoprotectant (e.g., glycerol, trehalose, or DMSO) concentration of about 5% to 15% is used and permits survival of a substantial fraction of isolated cells after freezing and thawing from cryogenic temperatures. One skilled in the art will recognize a cryoprotectant formulation can adjusted dependent on the cellular species to be preserved. For example, certain species (e.g., gamma proteobacteria) are sensitive to cryopreservation and lose considerable viability after few days in cryo-storage. In some embodiments, biological materials (such as microbial strains including bacteria and fungi) are refrigerated at temperatures of −20° C. or at −80° C., e.g., with use of laboratory freezers. In some embodiments, biological materials are stored using the vapor phase of liquid nitrogen that brings the temperature to −170° C.

### Methods of Use

Included within the scope of this disclosure are methods for treatment of musculoskeletal disorders including osteoporosis, osteopenia, Paget's disease, stunting, osteoarthritis, osteomyelitis, and delayed or non-union fractures.

These methods include treatment with a prebiotic composition (e.g., a composition comprising or consisting of FOS, GOS, or other appropriate polysaccharide), optionally in conjunction with a probiotic composition, one or more digestible saccharides (e.g. lactose, glucose, or galactose), a buffer, or a combination thereof. These methods optionally are used in combination with other treatments to reduce the musculoskeletal disorder. Any suitable treatment can be used. In some embodiments the additional treatment is administered before, during, or after treatment with a prebiotic composition, or any combination thereof. In an embodiment, when the musculoskeletal disorder or disorders are not completely or substantially completely eliminated by treatment with a prebiotic composition, the additional treatment is administered after prebiotic treatment is terminated. The additional treatment is used on an as-needed basis.

In an embodiment, a subject to be treated for one or more symptoms of a musculoskeletal disorder is a human. In an embodiment, the human subject is a preterm newborn, a full term newborn, an infant up to one year of age, a young child (e.g., 1 yr to 12 yrs), a teenager (e.g., 13-19 yrs), an adult (e.g., 20-64 yrs), a pregnant women, or an elderly adult (65 yrs and older).

In an embodiment, the condition to be treated is osteoporosis or osteopenia. In an embodiment, the condition to be treated is osteoporosis or osteopenia, and treating osteoporosis further involves administration of any one or combination of known anti-osteoporosis medications or treatments. These include, but are not limited to, bisphosphonates (alendronate, risedronate, ibandronate, zolendronate), biologics (denosumab, romosozumab), selective estrogen receptor mediators (Raloxifene), or anabolic agents (teriparatide, abaloparatide).

In an embodiment, the condition to be treated is osteoarthritis. In an embodiment, the condition to be treated is osteoarthritis, and treating the condition further involves administration of any one or combination of known anti-osteoarthritis medications or treatments. These include, but are not limited to, surgery, analgesics, non-steroidal anti-inflammatory drugs (aspirin, celecoxib, diclofenac, diflunisal, etodolac, ibuprofen, indomethacin, ketoprofen, ketorolac, nabumetone, naproxen, oxaprozin, piroxicam), menthol, weight loss regimens, physical exercise, acupuncture, narcotics (Codeine, Fentanyl, Hydrocodone, hydroporphone, meperidine, methadone, oxycodone), and physical therapy.

In an embodiment, the condition to be treated is a delayed or non-union fracture. In an embodiment, the condition to be treated is a delayed or non-union fracture, and treating the condition further involves administration of any one or combination of known treatments to improve delayed or non-union fractures. These include, but are not limited to surgical bone grafts or fixations and bone stimulation.

In an embodiment, the condition to be treated is osteomyelitis. The methods disclosed herein, optionally, are used in combination with other treatments to treat or prevent osteomyelitis. Typical treatments for osteomyelitis include, but are not limited to, intravenous or oral antibiotics (clindamycin, cefotetan, ticarcillin/clavulanate, ceftriaxone, metronidazole, piperacillin/tazobactam, fluoroquinolone, cefepime, ciprofloxacin, imipenem/cilastin, vancomycin, trimethoprim/sulfamethoxazole, minocycline, nafcillin, oxacillin, cefazolin, penicillin) and surgery. Any suitable treatment for osteomyelitis can be used. These include, but are not limited to, removal of diseased tissue and antibiotics, administered either orally or intravenously.

### Timing and Dose of Probiotics and Prebiotics

In an embodiment, probiotic bacteria, such as a Pediococcus species or a Leuconostoc species, are given prior to beginning treatment with a prebiotic. In an embodiment, probiotic bacteria, such as a Pediococcus species or a Leuconostoc species, are given in conjunction with treatment with a prebiotic (e.g., comprising or consisting essentially of FOS, GOS, or other appropriate polysaccharide), for part or all of the treatment with the prebiotic. Thus, in an embodiment, some or all doses of a prebiotic (e.g., comprising or consisting essentially of FOS, GOS, or other appropriate polysaccharide) are accompanied by a dose of bacteria, e.g., live cultured bacteria, e.g., a Pediococcus species or a Leuconostoc species. In an embodiment, bacteria, e.g., a Pediococcus species or a Leuconostoc species, are given initially with a prebiotic (e.g., comprising or consisting essentially of FOS, GOS, or other appropriate polysaccharide), but then use of the bacteria is discontinued. For example, the initial one, two, three, four, five, six, seven, eight, nine, ten, or more than ten days of treatment with a prebiotic (e.g., comprising or consisting essentially of FOS, GOS, or other appropriate polysaccharide) further comprises doses of bacteria, with the use of bacteria discontinued after that time. In an embodiment, bacteria, (e.g., bacteria in yogurt), or bacteria by themselves, can be given for the first two days of treatment; then the administration of bacteria is discontinued. In another embodiment, probiotic bacteria, either alone or in combination with other substances or treatments are used after the treatment with a prebiotic (comprising or consisting essentially of FOS, GOS, or other appropriate polysaccharide) is terminated. The bacteria can be taken for any suitable period after the termination of treatment with prebiotic and can be taken daily or at regular or irregular intervals. Doses can be as described below.

Any suitable amount of probiotic per serving can be used that allows an effective microbiota in the GI as demonstrated by an increase in bone mineral density, improved bone architecture, protection from loss of bone mineral density, improved bone turnover markers, or improvement in other markers of osteoporosis or osteopenia. Markers of osteoporosis or osteopenia can include elevated levels of Inflammatory cytokines in the blood including: Tumor necrosis factor alpha (TNFα), Interleukin-17 (IL-17), Interleukin-4 (IL-4), Interferon gamma (IFNγ), Receptor activator of nuclear factor kappa-B ligand (RANKL). They can also include increased one resorption blood markers (breakdown) crosslinked C-telopeptide of type 1 collagen (CTX), or decreased Bone formation blood markers: osteocalcin, alkaline phosphatase, N-terminal propeptide of type 1 collagen.

Any suitable amount of probiotic per serving can be used that allows an effective microbiota in the GI as demonstrated by an increase in healthy bone healing, including decreased incidence of delayed or non-union fractures or increased normal fracture callus formation. Markers of fracture healing defects include delayed healing, non-union fracture healing, or changes in fracture callus architecture (including increased size or adiposity of the fracture callus).

Typically, probiotics are given as live cultured bacteria. The dose can be 0.001 mg to 1 mg, or 0.5 mg to 5 mg, or 1 mg to 1000 mg, or 2 mg to 200 mg, or 2 mg to 100 mg, or 2 mg to 50 mg, or 4 mg to 25 mg, or 5 mg to 20 mg, or 10 mg to 15 mg, or 50 mg to 200 mg, or 200 mg to 1000 mg, or 10, 11, 12, 12.5, 13, 14, or 15 mg per serving. In an embodiment, L. acidophilus is used in a dose of 12.5 mg per serving. The probiotic bacteria can also be 0.5% w/w to 20% w/w of the final composition. The dose of probiotics can be given in combination with one or more prebiotics. Another common way of specifying the amount of probiotics is as a colony forming unit (cfu). In an embodiment, one or more strains of probiotic bacteria are ingested in an amount of between 1×10\hat{ }5 and 1×10\hat{ }12 cfu's per serving. In an embodiment, one or more strains of probiotic bacteria are ingested in an amount of 1×10\hat{ }5 to 1×10\hat{ }9 cfu's, or 1×10\hat{ }6 cfu's to 1×10\hat{ }10 cfu's, or 1×10\hat{ }6 cfu's to 1×10\hat{ }9 cfu's, or 1×10\hat{ }5 cfu's to 1×10\hat{ }6 cfu's, or 1×10\hat{ }5 cfu's to 1×10\hat{ }12 cfu's, or 1×10\hat{ }9 cfu's per serving. In another embodiment, one or more strains of probiotic bacteria are administered as part of a dairy product. In an embodiment, a typical serving size for a dairy product such as fluid milk is 240 g. In other embodiments, a serving size is 245 g, or 240 g to 245 g, or 227 to 300 g. In an embodiment the dairy product is yogurt. Yogurt can have a serving size of 4 oz, or 6 oz, or 8 oz, or 4 oz to 10 oz, or half cup, or 1 cup, or 113 g, or 170 g, or 227 g, or 245 g or 277 g, or 100 g to 350 g.

In an embodiment, probiotic bacteria are given as live cultured bacteria, e.g., in combination with a prebiotic (e.g., comprising or consisting essentially of FOS, GOS, or other appropriate polysaccharide) and, optionally, other substances. The dose can be 1 mg to 1000 mg, or 2 mg to 200 mg, or 2 mg to 100 mg, or 2 mg to 50 mg, or 4 mg to 25 mg, or 5 mg to 20 mg, or 10 mg to 15 mg, or 10, 11, 12, 12.5, 13, 14, or 15 mg of probiotic bacterial cell culture dry weight. In an embodiment, L. acidophilus is used in a dose of 12.5 mg. In an embodiment, as the administration of a prebiotic (e.g., comprising or consisting essentially of FOS, GOS, or other appropriate polysaccharide) dose to a subject increases, the dose of bacteria increases as well. For example, an initial dose of a prebiotic (e.g., comprising or consisting essentially of FOS, GOS, or other appropriate polysaccharides) can be 0.6 g to 1.0 g, e.g., 0.8 g, given in combination with 10-15 mg, e.g., 12.5 mg, of L. acidophilus. The dose of a prebiotic (e.g., comprising or consisting essentially of FOS, GOS, or other appropriate polysaccharide) can be increased incrementally by 0.6 g to 1.0 g, e.g., 0.8 g, and the accompanying dose of L. acidophilus can be increased by 10-15 mg, e.g., 12.5 mg, of L. acidophilus.

### FOS, GOS, or Other Appropriate Polysaccharide Formulations

### A. Formulations Introduction

In one aspect a prebiotic composition for the treatment of one or more musculoskeletal disorder is provided. In an embodiment a prebiotic composition comprises inulin, FOS, lactulose, GOS, raffinose, stachyose, or a combination thereof. In addition, other plant-derived polysaccharides such as xylan, pectin, isomalto-oligosaccharides, gentio-oligosaccharides, 4-O-methyl glucuronoxylan (GX), neutral arabinoxylan (AX), heteroxylan (HX) can be combined with the probiotics to enhance bacterial metabolic function. Some of these can be derived from plant material found in the plant host from which the probiotics were isolated from. Therefore, the probiotics are adapted to assimilate and digest the rich complexity and variety of polysaccharides present in the plant that play a role during digestion by the consumption of an animal.

In an embodiment a prebiotic composition comprises or consists of FOS, GOS, or other appropriate polysaccharide. In another embodiment a prebiotic composition comprises FOS, GOS, other, and one or more digestible saccharides. Digestible saccharides are saccharides that are digestible by humans and include, but are not limited to lactose, glucose, and galactose. In an embodiment a prebiotic composition comprises FOS, GOS, or other appropriate polysaccharide, and less than 20% weight/weight of one or more digestible saccharides (e.g. lactose, glucose, or galactose). In an embodiment a prebiotic composition comprises FOS, GOS, or other appropriate polysaccharide, and less than 10% of one or more digestible saccharides. In an embodiment a prebiotic composition comprises FOS, GOS, or other appropriate polysaccharide, and less than 5% of one or more digestible saccharides. In another embodiment a prebiotic composition contains less than 5% lactose. In another embodiment a prebiotic composition contains less than 4% lactose. In another embodiment a prebiotic composition contains less than 3% lactose. In another embodiment a prebiotic composition contains less than 2% lactose. In another embodiment a prebiotic composition contains less than 1% lactose. In another embodiment a prebiotic composition contains less than 0.5% lactose. In another embodiment a prebiotic composition contains less than 0.4% lactose. In another embodiment a prebiotic composition contains less than 0.3% lactose. In another embodiment a prebiotic composition contains less than 0.2% lactose. In another embodiment a prebiotic composition contains less than 0.1% lactose. In another embodiment a prebiotic composition contains less than 0.05% lactose. In another embodiment a prebiotic composition contains less than 0.01% lactose. In another embodiment a prebiotic composition contains less than 0.005% lactose. In an embodiment a prebiotic composition comprises FOS, GOS, or other appropriate polysaccharide, and essentially no lactose. In an embodiment a prebiotic composition does not contain any lactose. In another embodiment a prebiotic composition contains FOS, GOS, or other appropriate polysaccharide, and at least one probiotic bacteria strain. In another embodiment a prebiotic composition comprises FOS, GOS, or other appropriate polysaccharide, and optionally one or more of lactose, at least one probiotic bacteria strain, or a buffer. Additional ingredients include ingredients to improve handling, preservatives, antioxidants, flavorings and the like.

In an embodiment, a prebiotic composition comprises FOS, GOS, or other appropriate polysaccharide, or a probiotic. In other embodiment, a prebiotic composition is in the form of a powder, tablet, capsule, or liquid. In an embodiment, a prebiotic composition can be administered with a dairy product and is in the form of milk or other common dairy product such as a yogurt, shake, smoothie, cheese, and the like.

In embodiments where a prebiotic composition comprises less than 100% by weight of FOS, GOS, or other appropriate polysaccharide, the remaining ingredients can be any suitable ingredients intended for the consumption of the subject in need thereof, e.g., human, including, but not limited to, other prebiotics (e.g., FOS), a buffer, one or more digestible saccharides (e.g. lactose, glucose, or galactose), ingredients intended to inhibit clumping and increase pourability, such as silicone dioxide and microcrystalline cellulose, or similar ingredients as are well-known in the art. Remaining ingredients can also include ingredients to improve handling, preservatives, antioxidants, flavorings, and the like.

### B. Buffer Components

One or more buffers, optionally with a calcium counter ion, can also be administered in methods and compositions described herein. Any buffer suitable for consumption by the subject being treated, e.g., human, are useful for the compositions herein. The buffer neutralizes stomach acidity, which can, e.g., allow live bacteria to reach the gut. Buffers include citrates, phosphates, and the like. One embodiment utilizes a buffer with a calcium counter ion, such as Calcium Phosphate Tribasic. The calcium can serve to restore the calcium that many lactose intolerant subjects are missing in their diet. Calcium phosphate can protect Lactobacillus acidophilus from bile. Calcium phosphate can help neutralize stomach acidity.

In an embodiment, a buffer such as calcium phosphate is given prior to beginning treatment with a prebiotic composition (such as a composition comprising or consisting essentially of FOS, GOS, or other appropriate polysaccharide), optionally in conjunction with administration of bacteria. As used herein FOS indications one or more fructo-oligosaccharides and GOS indicates one or more galacto-oligosaccharides. In an embodiment, a buffer such as calcium phosphate is given in conjunction with treatment with a prebiotic composition (e.g., a composition comprising or consisting essentially of FOS, GOS, or other appropriate polysaccharide), for part or all of the treatment with lactose. Thus, in an embodiment, some or all doses of a prebiotic composition are accompanied by a dose of a buffer such as calcium phosphate. In an embodiment, a buffer such as calcium phosphate is given initially with a prebiotic composition (such as a composition comprising or consisting essentially of FOS, GOS, or other appropriate polysaccharide), but then its use is discontinued. For example, the initial one, two, three, four, five, six, seven, eight, nine, ten, or more than ten days of treatment with a prebiotic composition can include doses of a buffer such as calcium phosphate, with the use of the buffer discontinued after that time. In an embodiment, a buffer such as calcium phosphate can be given for the first two days of treatment, and then the administration of buffer is discontinued. In an embodiment, a buffer such as calcium phosphate, either alone or in combination with other substances or treatments is used after the treatment with a prebiotic composition is terminated. A buffer such as calcium phosphate can be taken for any suitable period after the termination of treatment with lactose, and can be taken daily or at regular or irregular intervals. Doses can be as described below.

Numerous buffers suitable for human consumption are known in the art, and any suitable buffer can be used in the methods and compositions described herein. Calcium triphosphate is an exemplary buffer, and its counterion supplies a nutrient that is often lacking in lactose-intolerant subjects, i.e. calcium. In an embodiment a buffer can be used in a dose from 2 mg to 2000 mg, or 4 mg to 400 mg, or 4 mg to 200 mg, or 4 mg to 100 mg, or 8 mg to 50 mg, or 10 mg to 40 mg, or 20 mg to 30 mg, or 20, 21, 22, 23, 24, 25, 26, 27, 28, 29, or 30 mg. In another embodiment a prebiotic composition further comprises an amount of a buffer from 1-50 mg, such as 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, or 50 mg. In an embodiment, buffer is used in a dose of 25 mg. In an embodiment, calcium phosphate is used in a dose of 25 mg. The dose can be given in combination with a prebiotic composition (e.g., a composition comprising or consisting essentially of FOS, GOS, or other appropriate polysaccharide). In an embodiment, as a prebiotic composition dose increases, the dose of buffer increases as well. For example, an initial dose of a prebiotic composition can be 0.6 g to 1.0 g, e.g., 0.8 g, given in combination with 20-30 mg, e.g., 25 mg, of buffer, e.g., calcium phosphate. The dose of a prebiotic composition can be increased incrementally by 0.6 g to 1.0 g, e.g., 0.8 g, and the accompanying dose of buffer, e.g., calcium phosphate, can be increased by 20-30 mg, e.g., 25 mg, of buffer, e.g., calcium phosphate.

### C. Compositions Comprising GOS and at Least One Probiotic Bacteria Strain

In an embodiment, a prebiotic composition comprises FOS, GOS, or other appropriate polysaccharide, and at least one probiotic bacteria strain. The FOS, GOS, or other appropriate polysaccharide can comprise more than 1% of the weight of the composition while the at least one probiotic bacteria strain will typically comprise less than 10%, 5%, 4%, 3%, or 2% by weight of the compositions. For example, the FOS, GOS, or other appropriate polysaccharide can be present at 1-99.75% by weight and the at least one probiotic bacteria strain at 0.25-2% by weight, or the FOS, GOS, or other appropriate polysaccharide can be present at 89-96% by weight and the bacteria at 1.2-3.7% by weight. In an embodiment, FOS, GOS, or other appropriate polysaccharide are present at 92% by weight and at least one probiotic bacteria strain, (e.g., L. mesenteroides, P. pentosaceus, Lactobacillus or other members from Table 4), is present at 1.5% by weight. In an embodiment, FOS, GOS, or other appropriate polysaccharide are present at 2% by weight and at least one probiotic bacteria strain, (e.g., L. mesenteroides, P. pentosaceus, or other members from Table 4), is present at 1.5% by weight. In another embodiment, FOS, GOS, or other appropriate polysaccharide are present at 93% by weight and at least one probiotic bacteria strain, (e.g., L. mesenteroides, P. pentosaceus, or other members from Table 4), is present at 1.5% by weight. In another embodiment, FOS, GOS, or other appropriate polysaccharide are present at 94% by weight and at least one probiotic bacteria strain, (e.g., L. mesenteroides, P. pentosaceus, or other members from Table 4), is present at 1.5% by weight. In another embodiment, FOS, GOS, or other appropriate polysaccharide are present at 95% by weight and at least one probiotic bacteria strain, (e.g., L. mesenteroides, P. pentosaceus, or other members from Table 4), is present at 1.5% by weight. In another embodiment, FOS, GOS, or other appropriate polysaccharide are present at 96% by weight and at least one probiotic bacteria strain, (e.g L. mesenteroides, P. pentosaceus, or other members from Table 4), is present at 1.5% by weight. In another embodiment, FOS, GOS, or other appropriate polysaccharide are present at 97% by weight and at least one probiotic bacteria strain, (e.g., L. mesenteroides, P. pentosaceus, or other members from Table 4), is present at 1.5% by weight. In another embodiment, FOS, GOS, or other appropriate polysaccharide are present at 98% by weight and at least one probiotic bacteria strain, (e.g., L. mesenteroides, P. pentosaceus, or other members from Table 4), is present at 1.5% by weight. In another embodiment, FOS, GOS, or other appropriate polysaccharide are present at 98.5% by weight and at least one probiotic bacteria strain, (e.g., L. mesenteroides, P. pentosaceus, or other members from Table 4), is present at 1.5% by weight. If the at least one probiotic bacteria strain and FOS, GOS, or other appropriate polysaccharide do not make up 100% by weight of the prebiotic composition, the remaining ingredients can be any suitable ingredients intended for consumption by the subject in need thereof, e.g., human, including, but not limited to, other prebiotics (e.g., FOS), one or more buffers, digestible saccharides (e.g. lactose, glucose, or galactose), ingredients intended to inhibit clumping and increase pourability, such as silicone dioxide and microcrystalline cellulose, or similar ingredients as are well-known in the art. Remaining ingredients can also include ingredients to improve handling, preservatives, antioxidants, flavorings and the like.

### D. Compositions Comprising FOS, GOS, or Other Appropriate Polysaccharide and a Buffer

In another embodiment, a prebiotic composition comprises FOS, GOS, or other appropriate polysaccharide and a buffer (e.g., calcium phosphate tribasic). For example, FOS, GOS, or other appropriate polysaccharide can be present at 1-100% by weight and the buffer at 0.50-4% by weight, or FOS, GOS, or other appropriate polysaccharide can be present at 1-96% by weight and the buffer at 1 to 3.75% by weight. In an embodiment, FOS, GOS, or other appropriate polysaccharide are present at 1% by weight and buffer is present at 3% by weight. In an embodiment, FOS, GOS, or other appropriate polysaccharide are present at 5% by weight and buffer is present at 3% by weight. In an embodiment, FOS, GOS, or other appropriate polysaccharide are present at 10% by weight and buffer is present at 3% by weight. In an embodiment, FOS, GOS, or other appropriate polysaccharide are present at 15% by weight and buffer is present at 15% by weight. In an embodiment, FOS, GOS, or other appropriate polysaccharide are present at 20% by weight and buffer is present at 3% by weight. In an embodiment, FOS, GOS, or other appropriate polysaccharide are present at 25% by weight and buffer is present at 3% by weight. In an embodiment, FOS, GOS, or other appropriate polysaccharide are present at 30% by weight and buffer is present at 3% by weight. In an embodiment, FOS, GOS, or other appropriate polysaccharide are present at 35% by weight and buffer is present at 3% by weight. In an embodiment, FOS, GOS, or other appropriate polysaccharide are present at 40% by weight and buffer is present at 3% by weight. In an embodiment, FOS, GOS, or other appropriate polysaccharide are present at 50% by weight and buffer is present at 3% by weight. In an embodiment, FOS, GOS, or other appropriate polysaccharide are present at 60% by weight and buffer is present at 3% by weight. In an embodiment, FOS, GOS, or other appropriate polysaccharide are present at 70% by weight and buffer is present at 3% by weight. In another embodiment, FOS, GOS, or other appropriate polysaccharide are present at 90% by weight and buffer is present at 3% by weight. In another embodiment, FOS, GOS, or other appropriate polysaccharide are present at 92% by weight and buffer is present at 3% by weight. In another embodiment, FOS, GOS, or other appropriate polysaccharide are present at 93% by weight and buffer is present at 3% by weight. In another embodiment, FOS, GOS, or other appropriate polysaccharide are present at 94% by weight and buffer is present at 3% by weight. In another embodiment, FOS, GOS, or other appropriate polysaccharide are present at 95% by weight and buffer is present at 3% by weight. In another embodiment, FOS, GOS, or other appropriate polysaccharide are present at 96% by weight and buffer is present at 3% by weight. In another embodiment, FOS, GOS, or other appropriate polysaccharide are present at 97% by weight and buffer is present at 2% by weight. In another embodiment, FOS, GOS, or other appropriate polysaccharide are present at 98% by weight and buffer is present at 1% by weight. In another embodiment, FOS, GOS, or other appropriate polysaccharide are present at 99% by weight and buffer is present at 1% by weight. In another embodiment, FOS, GOS, or other appropriate polysaccharide are present at 100% by weight and buffer is present at less than 1% by weight. If the buffer and FOS, GOS, or other appropriate polysaccharide do not make up 100% by weight of the composition, the remaining ingredients can be any suitable ingredients intended for consumption by the subject (e.g., a human) including, but not limited to, probiotics (e.g., beneficial bacteria) or other prebiotics (e.g., FOS), but also including ingredients intended to inhibit clumping and increase pourability, such as silicone dioxide and microcrystalline cellulose, or similar ingredients as are well-known in the art. Remaining ingredients can also include ingredients to improve handling, preservatives, antioxidants, flavorings and the like.

### E. Compositions Comprising a Digestible Saccharide, a Probiotic Bacteria, and FOS, GOS, or Other Appropriate Polysaccharide

In an embodiment, a prebiotic composition comprises a digestible saccharide (e.g. lactose, glucose, or galactose), at least one probiotic bacterium (e.g., L. mesenteroides, P. pentosaceus, or other members from Table 4), and FOS, GOS, or other appropriate polysaccharide. In an embodiment, lactose can be present at 1-20% by weight, bacteria at 0.25-20.10% by weight, and FOS, GOS, or other appropriate polysaccharide at 1-98.75% by weight. In another embodiment lactose can be present at 5-20% by weight, bacteria at 0.91-1.95% by weight, and FOS, GOS, or other appropriate polysaccharide at 1 to 96% by weight. In another embodiment, lactose is present at 20% by weight, bacteria at 1.5% by weight, and FOS, GOS, or other appropriate polysaccharide are present at 1% by weight. In another embodiment, lactose is present at 20% by weight, bacteria at 1.5% by weight, and FOS, GOS, or other appropriate polysaccharide are present at 50% by weight. In another embodiment, lactose is present at 20% by weight, bacteria at 1.5% by weight, and FOS, GOS, or other appropriate polysaccharide are present at 60% by weight. In another embodiment, lactose is present at 20% by weight, bacteria at 1.5% by weight, and FOS, GOS, or other appropriate polysaccharide are present at 70% by weight. In another embodiment, lactose is present at 5% by weight, bacteria at 1.5% by weight, and FOS, GOS, or other appropriate polysaccharide are present at 90% by weight. In another embodiment, lactose is present at 5% by weight, bacteria at 1.5% by weight, and FOS, GOS, or other appropriate polysaccharide are present at 92% by weight. In another embodiment, lactose is present at 5% by weight, bacteria at 1.5% by weight, and FOS, GOS, or other appropriate polysaccharide are present at 93% by weight. In another embodiment, lactose is present at 5% by weight, bacteria at 1% by weight, and FOS, GOS, or other appropriate polysaccharide are present at 94% by weight. In another embodiment, lactose is present at 4.5% by weight, bacteria at 1.5% by weight, and FOS, GOS, or other appropriate polysaccharide are present at 94% by weight. In another embodiment, lactose is present at 4.5% by weight, bacteria at 0.5% by weight, and FOS, GOS, or other appropriate polysaccharide are present at 95% by weight. In another embodiment, lactose is present at 3.5% by weight, bacteria at 0.5% by weight, and FOS, GOS, or other appropriate polysaccharide are present at 96% by weight. In another embodiment, lactose is present at 2.5% by weight, bacteria at 0.5% by weight, and FOS, GOS, or other appropriate polysaccharides are present at 97% by weight. In another embodiment, lactose is present at 1.5% by weight, bacteria at 0.5% by weight, and FOS, GOS, or other appropriate polysaccharide are present at 98% by weight. In another embodiment, lactose is present at 0.5% by weight, bacteria at 0.5% by weight, and FOS, GOS, or other appropriate polysaccharide are present at 99% by weight. If the bacteria, FOS, GOS, or other appropriate polysaccharide and lactose do not make up 100% of the composition, the remaining ingredients can be any suitable ingredients intended for consumption by the subject, e.g., a human, including, but not limited to a buffer, digestible saccharides (e.g., lactose, glucose, or galactose), ingredients intended to inhibit clumping and increase pourability, such as silicone dioxide and microcrystalline cellulose, or similar ingredients as are well-known in the art. Remaining ingredients can also include ingredients to improve handling, preservatives, antioxidants, flavorings and the like.

### F. Compositions Comprising FOS, GOS, or Other Appropriate Polysaccharide, a Probiotic Bacteria, and Buffer

In an embodiment, a prebiotic composition comprises FOS, GOS, or other appropriate polysaccharide, a probiotic bacteria strain, and buffer. In an embodiment, FOS, GOS, or other appropriate polysaccharide can be present at 1-100% by weight, a probiotic bacteria strain at 0.25-2% by weight, and the buffer at 0.50-4% by weight. In another embodiment, FOS, GOS, or other appropriate polysaccharide can be present at 1-95% by weight, a probiotic bacteria strain at 0.91-1.95% by weight, and the buffer at 1.2-30.75% by weight. In another embodiment, FOS, GOS, or other appropriate polysaccharide are present at 1% by weight, a probiotic bacteria strain at 1.5% by weight, and buffer is present at 3% by weight. In another embodiment, FOS, GOS, or other appropriate polysaccharide are present at 5% by weight, a probiotic bacteria strain at 1.5% by weight, and buffer is present at 3% by weight. In another embodiment, FOS, GOS, or other appropriate polysaccharide are present at 10% by weight, a probiotic bacteria strain at 1.5% by weight, and buffer is present at 3% by weight. In another embodiment, FOS, GOS, or other appropriate polysaccharide are present at 15% by weight, a probiotic bacteria strain at 1.5% by weight, and buffer is present at 3% by weight. In another embodiment, FOS, GOS, or other appropriate polysaccharide are present at 20% by weight, a probiotic bacteria strain at 1.5% by weight, and buffer is present at 3% by weight. In another embodiment, FOS, GOS, or other appropriate polysaccharide are present at 25% by weight, a probiotic bacteria strain at 1.5% by weight, and buffer is present at 3% by weight. In another embodiment, FOS, GOS, or other appropriate polysaccharide are present at 30% by weight, a probiotic bacteria strain at 1.5% by weight, and buffer is present at 3% by weight. In another embodiment, FOS, GOS, or other appropriate polysaccharide are present at 35% by weight, a probiotic bacteria strain at 1.5% by weight, and buffer is present at 3% by weight. In another embodiment, FOS, GOS, or other appropriate polysaccharide are present at 40% by weight, a probiotic bacteria strain at 1.5% by weight, and buffer is present at 3% by weight. In another embodiment, FOS, GOS, or other appropriate polysaccharide are present at 50% by weight, a probiotic bacteria strain at 1.5% by weight, and buffer is present at 3% by weight. In another embodiment, FOS, GOS, or other appropriate polysaccharide are present at 60% by weight, a probiotic bacteria strain at 1.5% by weight, and buffer is present at 3% by weight. In another embodiment, FOS, GOS, or other appropriate polysaccharide are present at 70% by weight, a probiotic bacteria strain at 1.5% by weight, and buffer is present at 3% by weight. In another embodiment, FOS, GOS, or other appropriate polysaccharide are present at 90% by weight, a probiotic bacteria strain at 1.5% by weight, and buffer is present at 3% by weight. In another embodiment, FOS, GOS, or other appropriate polysaccharide are present at 92% by weight, a probiotic bacteria strain at 1.5% by weight, and buffer is present at 3% by weight. In another embodiment, FOS, GOS, or other appropriate polysaccharide are present at 93% by weight, a probiotic bacteria strain at 1.5% by weight, and buffer is present at 3% by weight. In another embodiment, FOS, GOS, or other appropriate polysaccharide are present at 94% by weight, a probiotic bacteria strain at 1.5% by weight, and buffer is present at 3% by weight. In another embodiment, FOS, GOS, or other appropriate polysaccharide are present at 95% by weight, a probiotic bacteria strain at 1.5% by weight, and buffer is present at 3% by weight. In another embodiment, FOS, GOS, or other appropriate polysaccharide are present at 96% by weight, a probiotic bacteria strain at 1.5% by weight, and buffer is present at 2% by weight. In another embodiment, FOS, GOS, or other appropriate polysaccharide are present at 97% by weight, a probiotic bacteria strain at 1.5% by weight, and buffer is present at 1.5% by weight. In another embodiment, FOS, GOS, or other appropriate polysaccharide are present at 99% by weight, a probiotic bacteria strain at 0.5% by weight, and buffer is present at 0.5% by weight. In another embodiment, FOS, GOS, or other appropriate polysaccharide are present at 100% by weight, a probiotic bacteria strain at less than 0.5% by weight, and buffer is present at less than 0.5% by weight. If the probiotic bacteria strain, buffer, and FOS, GOS, or other appropriate polysaccharide do not make up 100% of the composition, the remaining ingredients can be any suitable ingredients intended for the consumption of a subject (e.g., human) including, but not limited to, other prebiotics (e.g., FOS), digestible saccharides (e.g., lactose, glucose or galactose), ingredients intended to inhibit clumping and increase pourability, such as silicone dioxide and microcrystalline cellulose, or similar ingredients as are well-known in the art. Remaining ingredients can also include ingredients to improve handling, preservatives, antioxidants, flavorings and the like.

### G. Compositions Comprising a Digestible Saccharide, FOS, GOS, or Other Appropriate Polysaccharide, and a Buffer

In an embodiment, a prebiotic composition comprises a digestible saccharide (e.g. lactose, glucose, or galactose), FOS, GOS, or other appropriate polysaccharide, and a buffer. For example, lactose can be present at 1-20% by weight, FOS, GOS, or other appropriate polysaccharide at 1-100% by weight, and the buffer at 0.50-4% by weight, or the lactose can be present at 5-20% by weight, FOS, GOS, or other appropriate polysaccharide at 1-96% by weight, and the buffer at 1.2-30.75% by weight. In an embodiment, lactose is present at 20% by weight, FOS, GOS, or other appropriate polysaccharide at 1% by weight, and buffer is present at 3% by weight. In an embodiment, lactose is present at 5% by weight, FOS, GOS, or other appropriate polysaccharide at 1% by weight, and buffer is present at 3% by weight. In an embodiment, lactose is present at 20% by weight, FOS, GOS, or other appropriate polysaccharide at 10% by weight, and buffer is present at 3% by weight. In an embodiment, lactose is present at 20% by weight, FOS, GOS, or other appropriate polysaccharide at 15% by weight, and buffer is present at 3% by weight. In an embodiment, lactose is present at 20% by weight, FOS, GOS, or other appropriate polysaccharide at 20% by weight, and buffer is present at 3% by weight. In an embodiment, lactose is present at 20% by weight, FOS, GOS, or other appropriate polysaccharide at 25% by weight, and buffer is present at 3% by weight. In an embodiment, lactose is present at 20% by weight, FOS, GOS, or other appropriate polysaccharide at 30% by weight, and buffer is present at 3% by weight. In an embodiment, lactose is present at 20% by weight, FOS, GOS, or other appropriate polysaccharide at 35% by weight, and buffer is present at 3% by weight. In an embodiment, lactose is present at 20% by weight, FOS, GOS, or other appropriate polysaccharide at 40% by weight, and buffer is present at 3% by weight. In an embodiment, lactose is present at 20% by weight, FOS, GOS, or other appropriate polysaccharide at 50% by weight, and buffer is present at 3% by weight. In an embodiment, lactose is present at 20% by weight, FOS, GOS, or other appropriate polysaccharide at 60% by weight, and buffer is present at 3% by weight. In an embodiment, lactose is present at 20% by weight, FOS, GOS, or other appropriate polysaccharide at 70% by weight, and buffer is present at 3% by weight. In another embodiment, lactose is present at 5% by weight, FOS, GOS, or other appropriate polysaccharide at 90% by weight, and buffer is present at 3% by weight. In another embodiment, lactose is present at 5% by weight, FOS, GOS, or other appropriate polysaccharide at 92% by weight, and buffer is present at 3% by weight. In another embodiment, lactose is present at 4% by weight, FOS, GOS, or other appropriate polysaccharide at 93% by weight, and buffer is present at 3% by weight. In another embodiment, lactose is present at 3% by weight, FOS, GOS, or other appropriate polysaccharide at 94% by weight, and buffer is present at 3% by weight. In another embodiment, lactose is present at 2% by weight, FOS, GOS, or other appropriate polysaccharide at 95% by weight, and buffer is present at 3% by weight. In another embodiment, lactose is present at 1% by weight, FOS, GOS, or other appropriate polysaccharide at 96% by weight, and buffer is present at 3% by weight. If a suitable prebiotic, buffer and lactose do not make up 100% of the composition by weight, the remaining ingredients can be any suitable ingredients intended for consumption by a subject (e.g., human) including, but not limited to, bacteria, ingredients intended to inhibit clumping and increase pourability, such as silicone dioxide and microcrystalline cellulose, or similar ingredients as are well-known in the art. Remaining ingredients can also include ingredients to improve handling, preservatives, antioxidants, flavorings and the like.

### H. Compositions Comprising a Digestible Saccharide, Bacteria, GOS, and a Buffer

In an embodiment, a composition comprises a digestible saccharide (e.g. lactose, glucose, or galactose), bacteria, FOS, GOS, or other appropriate polysaccharide, and buffer. For example, lactose can be present at 1-20% by weight, bacteria at 0.25-2.10% by weight, FOS, GOS, or other appropriate polysaccharide at 1-100% by weight, and the buffer at 0.50-4% by weight, or the lactose can be present at 5-20% by weight, bacteria at 0.91-1.95% by weight, FOS, GOS, or other appropriate polysaccharide at 70-95% by weight, and the buffer at 1.2-30.75% by weight. In an embodiment, lactose is present at 20% by weight, bacteria at 1.47% by weight, FOS, GOS, or other appropriate polysaccharide at 1% by weight, and buffer is present at 3% by weight. In an embodiment, lactose is present at 20% by weight, bacteria at 1.47% by weight, FOS, GOS, or other appropriate polysaccharide at 10% by weight, and buffer is present at 3% by weight. In an embodiment, lactose is present at 20% by weight, bacteria at 1.47% by weight, FOS, GOS, or other appropriate polysaccharide at 15% by weight, and buffer is present at 3% by weight. In an embodiment, lactose is present at 20% by weight, bacteria at 1.47% by weight, FOS, GOS, or other appropriate polysaccharide at 20% by weight, and buffer is present at 3% by weight. In an embodiment, lactose is present at 20% by weight, bacteria at 1.47% by weight, FOS, GOS, or other appropriate polysaccharide at 25% by weight, and buffer is present at 3% by weight. In an embodiment, lactose is present at 20% by weight, bacteria at 1.47% by weight, FOS, GOS, or other appropriate polysaccharide at 30% by weight, and buffer is present at 3% by weight. In an embodiment, lactose is present at 20% by weight, bacteria at 1.47% by weight, FOS, GOS, or other appropriate polysaccharide at 35% by weight, and buffer is present at 3% by weight. In an embodiment, lactose is present at 20% by weight, bacteria at 1.47% by weight, FOS, GOS, or other appropriate polysaccharide at 40% by weight, and buffer is present at 3% by weight. In an embodiment, lactose is present at 20% by weight, bacteria at 1.47% by weight, FOS, GOS, or other appropriate polysaccharide at 50% by weight, and buffer is present at 3% by weight. In an embodiment, lactose is present at 20% by weight, bacteria at 1.47% by weight, FOS, GOS, or other appropriate polysaccharide at 60% by weight, and buffer is present at 3% by weight. In an embodiment, lactose is present at 20% by weight, bacteria at 1.47% by weight, FOS, GOS, or other appropriate polysaccharide at 70% by weight, and buffer is present at 3% by weight. In an embodiment, lactose is present at 5% by weight, bacteria at 1.47% by weight, FOS, GOS, or other appropriate polysaccharide at 90% by weight, and buffer is present at 3% by weight. In an embodiment, lactose is present at 3% by weight, bacteria at 1.47% by weight, FOS, GOS, or other appropriate polysaccharide at 92% by weight, and buffer is present at 3% by weight. In an embodiment, lactose is present at 2% by weight, bacteria at 1.47% by weight, FOS, GOS, or other appropriate polysaccharide at 93% by weight, and buffer is present at 3% by weight. In an embodiment, lactose is present at 1% by weight, bacteria at 1.47% by weight, FOS, GOS, or other appropriate polysaccharide at 94% by weight, and buffer is present at 3% by weight. In an embodiment, lactose is present at 0.5% by weight, bacteria at 1.47% by weight, FOS, GOS, or other appropriate polysaccharide at 95% by weight, and buffer is present at 3% by weight. If the bacteria, FOS, GOS, or other, buffer and lactose do not make up 100% of the composition by weight, the remaining ingredients can be any suitable ingredients intended for consumption by a subject, e.g., human, including, but not limited to, ingredients intended to inhibit clumping and increase pourability, such as silicone dioxide and microcrystalline cellulose, or similar ingredients as are well-known in the art. Remaining ingredients can also include ingredients to improve handling, preservatives, antioxidants, flavorings and the like.

### I. Additional Ingredients

Additional ingredients include ingredients to improve handling, preservatives, antioxidants, flavorings and the like. For example, in an embodiment, a prebiotic composition in powdered form can include flavorings such that when mixed in a liquid (e.g., water), the powder can flavor the liquid with various flavors such as grape, strawberry, lime, lemon, chocolate, and the like. In an embodiment, the compositions include microcrystalline cellulose or silicone dioxide. Preservatives can include, for example, benzoic acid, alcohols, for example, ethyl alcohol, and hydroxybenzoates. Antioxidants can include, for example, butylated hydroxyanisole (BHA), butylated hydroxytoluene (BHT), tocopherols (e.g., Vitamin E), and ascorbic acid (Vitamin C).

### Timing and Dosage of Probiotic and Treatments Known to Combat Musculoskeletal Disorders

In an embodiment, probiotic microbes, such as L. mesenteroides and P. pentosaceus, are given prior to beginning treatment with a drug typically prescribed for treatment of a musculoskeletal disorder.

Thus, in an embodiment, some or all doses of a treatment or drug are accompanied by a dose of microbes, e.g., live cultured bacteria or yeast, e.g., L. mesenteroides, P. pentosaceus. In an embodiment, microbes, e.g., L. mesenteroides, P. pentosaceus, are given initially with another treatment or drug, but then use of the microbes is discontinued. For example, the initial one, two, three, four, five, six, seven, eight, nine, ten, or more than ten days of treatment with a treatment or drug further comprises doses of microbes, with the use of microbes discontinued after that time. In an embodiment, microbes, (e.g., bacteria in yogurt), or microbes by themselves, can be given for the first two days of treatment; then the administration of microbes is discontinued. In another embodiment, probiotic microbes, either alone or in combination with other substances or treatments are used after the treatment with a drug or treatment for musculoskeletal disorders is terminated. The microbes can be taken for any suitable period after the termination of treatment with the drug and can be taken daily or at regular or irregular intervals. Doses can be as described below. Any suitable amount of probiotic per serving can be used that allows an effective microbiota in the GI as demonstrated by, for example, decreased symptoms of a given musculoskeletal disorder.

Examples of anti-osteoprosis combination partners are but are not limited to, bisphosphonates (alendronate, risedronate, ibandronate, zolendronate), biologics (denosumab, romosozumab), selective estrogen receptor mediators (Raloxifene), or anabolic agents (teriparatide, abaloparatide). In an embodiment, probiotic microbes, such as L. mesenteroides, P. pentosaceus, are given in conjunction with treatment, such as, but are not limited to, bisphosphonates (alendronate, risedronate, ibandronate, zolendronate), biologics (denosumab, romosozumab), selective estrogen receptor mediators (Raloxifene), or anabolic agents (teriparatide, abaloparatide).

Examples of anti-osteoarthritis combination partners are surgery, analgesics, non-steroidal anti-inflammatory drugs, menthol, weight loss regimens, physical exercise, acupuncture, narcotics, teriparatide, abaloparatide, and physical therapy.

Examples of treatments for osteomyelitis that may be used in combination with compositions disclosed herein, include, but are not limited to surgery and antibiotics. In some embodiments, antibiotics are given intravenously. In some embodiments, antibiotics are given orally. Typically, compositions disclosed herein are given after cessation of antibiotic therapy; however, in some cases, a suitable antibiotic or a suitable delivery route of antibiotic allows for concurrent use of compositions described herein and antibiotic therapy.

Examples of treatments for delayed or non-union fractures include bone stimulation and surgery, such as bone grafts or fixations.

### Dosage Forms

### A. General

Compositions described herein include any suitable form, including liquid or powder. Powdered compositions can be as pure powder, or can be in the form of capsules, tablets, or the like. Powder can be packaged in bulk (e.g., in a container containing sufficient prebiotic or other substances for a subject to follow for an entire course of treatment with increasing doses of prebiotic, or a portion of a course of treatment), or as individual packets (e.g., packets containing a single dose of prebiotic plus other components, or packets containing the dose of prebiotic and other components needed for a particular day of a prebiotic treatment regimen). If packaged in bulk, the powder can be in any suitable container, such as a packet, sachet, canister, ampoule, ramekin, or bottle. The container can also include one or more scoops or similar serving devices of a size or sizes appropriate to measure and serve one or more doses of prebiotic and, optionally, other ingredients included in the powder. Liquid compositions contain prebiotic and, optionally, other ingredients, in a suitable liquid, e.g., water or buffer. Liquid compositions can be provided in bulk (e.g., in a container containing sufficient prebiotic or other substances for one subject in need thereof to follow an entire course of treatment with increasing doses of prebiotic, or a portion of a course of treatment), or as individual containers, such as cans, bottles, soft packs, and the like (e.g., containers containing a single dose of prebiotic plus other components in suitable liquid, or containers containing the dose of prebiotic and other components needed for a particular day of a prebiotic treatment regimen). The container can also include one or more measuring cups or similar serving devices of a size or sizes appropriate to measure and serve one or more doses of prebiotic and, optionally, other ingredients included in the liquid.

In an embodiment, compositions described herein comprise one or more excipients. In an embodiment, the one or more excipients comprise one or more antiadherents, one or more binders, one or more coatings, one or more disintegrants, one or more fillers, one or more flavors, one or more colors, one or more lubricants, one or more glidants, one or more sorbents, one or more preservatives, one or more sweeteners, or a combination thereof. In an embodiment, the antiadherent is magnesium stearate. In an embodiment, the one or more binders are cellulose, microcrystalline cellulose, hydroxypropyl cellulose, xylitol, sorbitol, maltitol, gelatin, polyvinylpyrrolidone, polyethylene glycol, methyl cellulose, hydroxypropyl methylcellulose, or a combination thereof. In an embodiment, the one or more coatings are a hydroxypropyl methylcellulose film, shellac, corn protein zein, gelatin, methyl acrylate-methacrylic acid copolymers, cellulose acetate succinate, hydroxy propyl methyl cellulose phthalate, hydroxy propyl methyl cellulose acetate succinate, polyvinyl acetate phthalate, methyl methacrylate-methacrylic acid copolymers, sodium alginate, stearic acid, or a combination thereof. In an embodiment, the one or more disintegrants are crosslinked polyvinylpyrrolidone (crospovidone), crosslinked sodium carboxymethyl cellulose (croscarmellose sodium), sodium starch glycolate, or a combination thereof. In an embodiment, the one or more fillers are calcium carbonate, magnesium stearate, dibasic calcium phosphate, cellulose, vegetable oil, vegetable fat, or a combination thereof. In an embodiment, the one or more flavors are mint, cherry, anise, peach, apricot, licorice, raspberry, vanilla, or a combination thereof. In an embodiment, the one or more lubricants are talc, silica, vegetable stearin, magnesium stearate, stearic acid, or a combination thereof. In an embodiment, the one or more glidants are fumed silica, talc, magnesium carbonate, or a combination thereof. In an embodiment, the one or more sorbents are fatty acids, waxes, shellac, plastics, plant fibers, or a combination thereof. In an embodiment, the one or more preservatives are vitamin A, vitamin E, vitamin C, retinyl palmitate, selenium, cysteine, methionine, citric acid, sodium citrate, methyl paraben, propyl paraben, or a combination thereof. In an embodiment, the one or more sweeteners are stevia, aspartame, sucralose, neotame, acesulfame potassium, saccharin or a combination thereof.

### B. Oral Dosage Forms and Components

In one aspect provided herein are methods and compositions formulated for oral delivery to a subject in need thereof. In an embodiment a composition is formulated to deliver a composition comprising a prebiotic to a subject in need thereof. In another embodiment, a pharmaceutical composition is formulated to deliver a composition comprising a prebiotic to a subject in need thereof. In another embodiment a composition is formulated to deliver a composition comprising prebiotic and a probiotic to a subject in need thereof.

### 1. Forms

In an embodiment, a composition is administered in solid, semi-solid, micro-emulsion, gel, or liquid form. Examples of such dosage forms include tablet forms disclosed in U.S. Pat. Nos. 3,048,526, 3,108,046, 4,786,505, 4,919,939, and 4,950,484; gel forms disclosed in U.S. Pat. Nos. 4,904,479, 6,482,435, 6,572,871, and 5,013,726; capsule forms disclosed in U.S. Pat. Nos. 4,800,083, 4,532,126, 4,935,243, and 6,258,380; or liquid forms disclosed in U.S. Pat. Nos. 4,625,494, 4,478,822, and 5,610,184; each of which is incorporated herein by reference in its entirety.

Forms of the compositions that can be used orally include tablets, push-fit capsules made of gelatin, as well as soft, sealed capsules made of gelatin and a plasticizer, such as glycerol or sorbitol. Tablets can be made by compression or molding, optionally with one or more accessory ingredients including freeze-dried plant material serving both as prebiotic and as a filler. Compressed tablets can be prepared by compressing in a suitable machine the active ingredient in a free-flowing form such as a powder or granules, optionally mixed with binders (e.g., povidone, gelatin, hydroxypropylmethyl cellulose), inert diluents, preservative, antioxidant, disintegrant (e.g., sodium starch glycolate, cross-linked povidone, cross-linked sodium carboxymethyl cellulose) or lubricating, surface active or dispersing agents. Molded tablets can be made by molding in a suitable machine a mixture of the powdered compound moistened with an inert liquid diluent. The tablets can optionally be coated or scored and can be formulated so as to provide slow or controlled release of the active ingredient therein. Tablets can optionally be provided with an enteric coating, to provide release in parts of the gut (e.g., colon, lower intestine) other than the stomach. All formulations for oral administration can be in dosages suitable for such administration. The push-fit capsules can contain the active ingredients in admixture with filler such as lactose, binders such as starches, and/or lubricants such as talc or magnesium stearate and, optionally, stabilizers. In soft capsules, the active compounds (prebiotics or probiotics) can be dissolved or suspended in suitable liquids, such as fatty oils, liquid paraffin, or liquid polyethylene glycols. In addition, stabilizers can be added. Dragee cores are provided with suitable coatings. For this purpose, concentrated sugar solutions can be used, which can optionally contain gum arabic, talc, polyvinyl pyrrolidone, carbopol gel, polyethylene glycol, or titanium dioxide, lacquer solutions, and suitable organic solvents or solvent mixtures. Dyestuffs or pigments can be added to the tablets or Dragee coatings for identification or to characterize different combinations of active compound doses.

Formulations for oral use can also be presented as hard gelatin capsules wherein the active ingredient is mixed with an inert solid diluent, for example, calcium carbonate, calcium phosphate or kaolin, or as soft gelatin capsules wherein the active ingredient is mixed with water soluble carrier such as polyethylene glycol or an oil medium, for example peanut oil, liquid paraffin, or olive oil.

Oral liquid preparations can be in the form of, for example, aqueous or oily suspensions, solutions, emulsions syrups or elixirs, or can be presented as a dry product for reconstitution with water or other suitable vehicle before use. Such liquid preparations can contain conventional additives, such as suspending agents, for example sorbitol, methyl cellulose, glucose syrup, gelatin, hydroxyethyl cellulose, carboxymethyl cellulose, aluminum stearate gel or hydrogenated edible fats, emulsifying agents, for example lecithin, sorbitan monooleate, acacia; nonaqueous vehicles (which can include edible oils), for example almond oil, oily esters such as glycerine, propylene glycol, or ethyl alcohol; preservatives, for example methyl or propyl p-hydoxybenzoate or sorbic acid, and, if desired, conventional flavoring or coloring agents.

In an embodiment, a provided composition includes a softgel formulation. A softgel can contain a gelatin-based shell that surrounds a liquid fill. The shell can be made of gelatin, plasticiser (e.g., glycerin and/or sorbitol), modifier, water, color, antioxidant, or flavor. The shell can be made with starch or carrageenan. The outer layer can be enteric coated. In an embodiment, a softgel formulation can include a water or oil soluble fill solution, or suspension of a composition, for example, a prebiotic composition, covered by a layer of gelatin.

An enteric coating can control the location of where a prebiotic composition is absorbed in the digestive system. For example, an enteric coating can be designed such that a prebiotic composition does not dissolve in the stomach but rather travels to the small intestine, where it dissolves. An enteric coating can be stable at low pH (such as in the stomach) and can dissolve at higher pH (for example, in the small intestine). Material that can be used in enteric coatings includes, for example, alginic acid, cellulose acetate phthalate, plastics, waxes, shellac, and fatty acids (e.g., stearic acid, palmitic acid). Enteric coatings are described, for example, in U.S. Pat. Nos. 5,225,202, 5,733,575, 6,139,875, 6,420,473, 6,455,052, and 6,569,457, all of which are herein incorporated by reference in their entirety. The enteric coating can be an aqueous enteric coating. Examples of polymers that can be used in enteric coatings include, for example, shellac (trade name EmCoat 120 N, Marcoat 125); cellulose acetate phthalate (trade name aquacoat CPD®, Sepifilm™ LP, Klucel, Aquacoat® ECD, and Metolose®); polyvinylacetate phthalate (trade name Sureteric®); and methacrylic acid (trade name Eudragit®).

In an embodiment, an enteric coated prebiotic composition is administered to a subject. In another embodiment, an enteric coated probiotic composition is administered to a subject. In another embodiment, an enteric coated probiotic and prebiotic composition is administered to a subject. In an embodiment, probiotic bacteria can be administered to a subject using an enteric coating. The stomach has an acidic environment that can kill probiotics. An enteric coating can protect probiotics as they pass through the stomach and small intestine.

Enteric coatings can be used to (1) prevent the gastric juice from reacting with or destroying the active substance, (2) prevent dilution of the active substance before it reaches the intestine, (3) ensure that the active substance is not released until after the preparation has passed the stomach, and (4) prevent live bacteria contained in the preparation from being killed because of the low pH-value in the stomach.

Enteric coatings can also be used for avoiding irritation of or damage to the mucous membrane of the stomach caused by substances contained in the oral preparation, and for counteracting or preventing formation or release of substances having an unpleasant odor or taste in the stomach. Finally, such coatings can be used for preventing nausea or vomiting on intake of oral preparations.

In an embodiment a prebiotic composition is provided as a tablet, capsule, or caplet with an enteric coating. In an embodiment the enteric coating is designed to hold the tablet, capsule, or caplet together when in the stomach. The enteric coating is designed to hold together in acid conditions of the stomach and break down in non-acid conditions and therefore release the drug in the intestines.

Softgel delivery systems can also incorporate phospholipids or polymers or natural gums to entrap a composition, for example, a prebiotic composition, in the gelatin layer with an outer coating to give desired delayed/control release effects, such as an enteric coating.

### Formulations of Softgel Fills can be at pH 2.5-7.5.

A softgel formulation can be sealed tightly in an automatic manner. A softgel formulation can easily be swallowed, allow for product identification using colors and several shapes, allow uniformity, precision and accuracy between dosages, be safe against adulteration, provide good availability and rapid absorption, and offer protection against contamination, light and oxidation. Furthermore, softgel formulations can avoid unpleasant flavors due to content encapsulation.

A composition comprising a softgel formulation can be in any of number of different sizes, including, for example, round, oblong, oval, tube, droplet, or suppositories.

In an embodiment a composition is provided in a dosage form which comprises an effective amount of prebiotic and one or more release controlling excipients as described herein. Suitable modified release dosage vehicles include, but are not limited to, hydrophilic or hydrophobic matrix devices, water-soluble separating layer coatings, enteric coatings, osmotic devices, multi-particulate devices, and combinations thereof. In an embodiment the dosage form is a tablet, caplet, capsule or lollipop. In another embodiment, the dosage form is a liquid, oral suspension, oral solution, or oral syrup. In yet another embodiment, the dosage form is a gel capsule, soft gelatin capsule, or hard gelatin capsule.

In an embodiment, the dosage form is a gelatin capsule having a size indicated in Table 1.

TABLE 1
Gel Cap Sizes Allowable For Human Consumption
Empty Gelatin Capsule Physical Specifications

| | Outer Diameter Size | Height or Locked Length | Actual Volume |
|---|---|---|---|
| | (mm) | (mm) | (ml) |
| 000 | 9.97 | 26.14 | 1.37 |
| 00 | 8.53 | 23.30 | 0.95 |
| 0 | 7.65 | 21.7 | 0.68 |
| 1 | 6.91 | 19.4 | 0.50 |
| 2 | 6.35 | 18.0 | 0.37 |
| 3 | 5.82 | 15.9 | 0.3 |
| 4 | 5.31 | 14.3 | 0.21 |
| 5 | 4.91 | 11.1 | 0.13 |

Note:
sizes and volumes are approximate.

In another embodiment a composition comprising a prebiotic is provided in effervescent dosage forms. The compositions can also comprise non-release controlling excipients.

In another embodiment, a composition comprising a prebiotic is provided in a dosage form that has at least one component that can facilitate release of the prebiotic. In a further embodiment the dosage form can be capable of giving a discontinuous release of the compound in the form of at least two consecutive pulses separated in time from 0.1 up to 24 hours. The compositions can comprise one or more release controlling and non-release controlling excipients, such as those excipients suitable for a disruptable semi-permeable membrane and as swellable substances.

In another embodiment the prebiotic mixture is a plant or plant extract, either in solid or liquid form.

In another embodiment a composition comprising a prebiotic is provided in an enteric coated dosage form. The composition can also comprise non-release controlling excipients.

In another embodiment a composition comprising a prebiotic is provided in a dosage form for oral administration to a subject in need thereof, which comprises one or more pharmaceutically acceptable excipients or carriers, enclosed in an intermediate reactive layer comprising a gastric juice-resistant polymeric layered material partially neutralized with alkali and having cation exchange capacity and a gastric juice-resistant outer layer.

In an embodiment a composition comprising a prebiotic is provided in the form of enteric-coated granules, for oral administration. The compositions can further comprise cellulose, disodium hydrogen phosphate, hydroxypropyl cellulose, hypromellose, lactose, mannitol, and sodium lauryl sulfate.

In another embodiment a composition comprising a prebiotic is provided in the form of enteric-coated pellets, for oral administration. The compositions can further comprise glyceryl monostearate 40-50, hydroxypropyl cellulose, hypromellose, magnesium stearate, methacrylic acid copolymer type C, polysorbate 80, sugar spheres, talc, and triethyl citrate.

In an embodiment a composition comprising a prebiotic is provided in the form of enteric-coated granules, for oral administration. The compositions can further comprise carnauba wax, crospovidone, diacetylated monoglycerides, ethylcellulose, hydroxypropyl cellulose, hypromellose phthalate, magnesium stearate, mannitol, sodium hydroxide, sodium stearyl fumarate, talc, titanium dioxide, and yellow ferric oxide.

In another embodiment a composition comprising a prebiotic can further comprise calcium stearate, crospovidone, hydroxypropyl methylcellulose, iron oxide, mannitol, methacrylic acid copolymer, polysorbate 80, povidone, propylene glycol, sodium carbonate, sodium lauryl sulfate, titanium dioxide, and triethyl citrate.

The compositions provided herein can be in unit-dosage forms or multiple-dosage forms. Unit-dosage forms, as used herein, refer to physically discrete units suitable for administration to human or non-human animal subject in need thereof and packaged individually. Each unit-dose can contain a predetermined quantity of an active ingredient(s) sufficient to produce the desired therapeutic effect, in association with other pharmaceutical carriers or excipients. Examples of unit-dosage forms include, but are not limited to, ampoules, syringes, and individually packaged tablets and capsules. Unit-dosage forms can be administered in fractions or multiples thereof. A multiple-dosage form is a plurality of identical unit-dosage forms packaged in a single container, which can be administered in segregated unit-dosage form. Examples of multiple-dosage forms include, but are not limited to, vials, bottles of tablets or capsules, or bottles of pints or gallons. In another embodiment the multiple dosage forms comprise different pharmaceutically active agents. For example a multiple dosage form can be provided which comprises a first dosage element comprising a composition comprising a prebiotic and a second dosage element comprising lactose or a probiotic, which can be in a modified release form.

In this example a pair of dosage elements can make a single unit dosage. In an embodiment a kit is provided comprising multiple unit dosages, wherein each unit comprises a first dosage element comprising a composition comprising a prebiotic and a second dosage element comprising probiotic, lactose or both, which can be in a modified release form. In another embodiment the kit further comprises a set of instructions.

In an embodiment, compositions can be formulated in various dosage forms for oral administration. The compositions can also be formulated as a modified release dosage form, including immediate-, delayed-, extended-, prolonged-, sustained-, pulsatile-, controlled-, extended, accelerated-, fast-, targeted-, programmed-release, and gastric retention dosage forms. These dosage forms can be prepared according to known methods and techniques (see, Remington: The Science and Practice of Pharmacy, supra; Modified-Release Drug Delivery Technology, Rathbone et al., Eds., Drugs and the Pharmaceutical Science, Marcel Dekker, Inc.: New York, N.Y., 2002; Vol. 126, which is herein incorporated by reference in its entirety).

In an embodiment, the compositions are in one or more dosage forms. For example, a composition can be administered in a solid or liquid form. Examples of solid dosage forms include but are not limited to discrete units in capsules or tablets, as a powder or granule, or present in a tablet conventionally formed by compression molding. Such compressed tablets can be prepared by compressing in a suitable machine the three or more agents and a pharmaceutically acceptable carrier. The molded tablets can be optionally coated or scored, having indicia inscribed thereon and can be so formulated as to cause immediate, substantially immediate, slow, controlled or extended release of a composition comprising a prebiotic. Furthermore, dosage forms of the invention can comprise acceptable carriers or salts known in the art, such as those described in the Handbook of Pharmaceutical Excipients, American Pharmaceutical Association (1986), incorporated by reference herein in its entirety.

In an embodiment, an effective amount of a composition comprising a prebiotic is mixed with a pharmaceutical excipient to form a solid preformulation composition comprising a homogeneous mixture of compounds described herein. When referring to these compositions as “homogeneous,” it is meant that the agents are dispersed evenly throughout the composition so that the composition can be subdivided into unit dosage forms such as tablets, caplets, or capsules. This solid preformulation composition can then be subdivided into unit dosage forms of the type described above comprising from, for example, 1 g to 20 mg of a prebiotic composition. A prebiotic composition can be formulated, in the case of caplets, capsules or tablets, to be swallowed whole, for example with water.

The compositions described herein can be in liquid form. The liquid formulations can comprise, for example, an agent in water-in-solution and/or suspension form; and a vehicle comprising polyethoxylated castor oil, alcohol, and/or a polyoxyethylated sorbitan mono-oleate with or without flavoring. Each dosage form comprises an effective amount of an active agent and can optionally comprise pharmaceutically inert agents, such as conventional excipients, vehicles, fillers, binders, disintegrants, pH adjusting substances, buffer, solvents, solubilizing agents, sweeteners, coloring agents, and any other inactive agents that can be included in pharmaceutical dosage forms for oral administration. Examples of such vehicles and additives can be found in Remington's Pharmaceutical Sciences, 17th edition (1985).

### 2. Manufacturing

The dosage forms described herein can be manufactured using processes that are well known to those of skill in the art. For example, for the manufacture of tablets, an effective amount of a prebiotic can be dispersed uniformly in one or more excipients, for example, using high shear granulation, low shear granulation, fluid bed granulation, or by blending for direct compression. Excipients include diluents, binders, disintegrants, dispersants, lubricants, glidants, stabilizers, surfactants and colorants. Diluents, also termed “fillers,” can be used to increase the bulk of a tablet so that a practical size is provided for compression. Non-limiting examples of diluents include lactose, cellulose, microcrystalline cellulose, mannitol, dry starch, hydrolyzed starches, powdered sugar, talc, sodium chloride, silicon dioxide, titanium oxide, dicalcium phosphate dihydrate, calcium sulfate, calcium carbonate, alumina and kaolin. Binders can impart cohesive qualities to a tablet formulation and can be used to help a tablet remain intact after compression. Non-limiting examples of suitable binders include starch (including corn starch and pregelatinized starch), gelatin, sugars (e.g., glucose, dextrose, sucrose, lactose and sorbitol), celluloses, polyethylene glycol, waxes, natural and synthetic gums, e.g., acacia, tragacanth, sodium alginate, and synthetic polymers such as polymethacrylates and polyvinylpyrrolidone. Lubricants can also facilitate tablet manufacture; non-limiting examples thereof include magnesium stearate, calcium stearate, stearic acid, glyceryl behenate, and polyethylene glycol. Disintegrants can facilitate tablet disintegration after administration, and non-limiting examples thereof include starches, alginic acid, crosslinked polymers such as, e.g., crosslinked polyvinylpyrrolidone, croscarmellose sodium, potassium or sodium starch glycolate, clays, celluloses, starches, gums and the like. Non-limiting examples of suitable glidants include silicon dioxide, talc, and the like. Stabilizers can inhibit or retard drug decomposition reactions, including oxidative reactions. Surfactants can also include and can be anionic, cationic, amphoteric or nonionic. If desired, the tablets can also comprise nontoxic auxiliary substances such as pH buffering agents, preservatives, e.g., antioxidants, wetting or emulsifying agents, solubilizing agents, coating agents, flavoring agents, and the like.

In an embodiment, a softgel formulation is made with a gelatin mass for the outer shell, and a composition including one or more substances, for example prebiotics and/or probiotics, for the capsule fill can be prepared. To make the gelatin mass, gelatin powder can be mixed with water and glycerin, heated, and stirred under vacuum. Additives, for example, flavors or colors, can be added to molten gelatin using a turbine mixer and transferred to mobile vessels. The gelatin mass can be kept in a steam-jacketed storage vessel at a constant temperature.

The encapsulation process can begin when the molten gel is pumped to a machine and two thin ribbons of gel are formed on either side of machine. These ribbons can then pass over a series of rollers and over a set of die that determine the size and shapes of capsules. A fill composition, for example a prebiotic and/or probiotic fill composition, can be fed to a positive displacement pump, which can dose the fill and inject it between two gelatin ribbons prior to sealing them together through the application of heat and pressure. To remove excess water, the capsules can pass through a conveyer into tumble dryers where a portion of the water can be removed. The capsules can then be placed on, for example, trays, which can be stacked and transferred into drying rooms. In the drying rooms, dry air can be forced over capsules to remove any excess moisture.

### 3. Release Formulations

Immediate-release formulations of an effective amount of a prebiotic composition can comprise one or more combinations of excipients that allow for a rapid release of a pharmaceutically active agent (such as from 1 minute to 1 hour after administration). In an embodiment an excipient can be microcrystalline cellulose, sodium carboxymethyl cellulose, sodium starch glycolate, corn starch, colloidal silica, Sodium Laurel Sulphate, Magnesium Stearate, Prosolve SMCC (HD90), croscarmellose Sodium, Crospovidone NF, Avicel PH200, and combinations of such excipients.

“Controlled-release” formulations (also referred to as sustained release (SR), extended-release (ER, XR, or XL), time-release or timed-release, controlled-release (CR), or continuous-release) refer to the release of a prebiotic composition from a dosage form at a particular desired point in time after the dosage form is administered to a subject. Controlled-release formulations can include one or more excipients, including but not limited to microcrystalline cellulose, sodium carboxymethyl cellulose, sodium starch glycolate, corn starch, colloidal silica, Sodium Laurel Sulphate, Magnesium Stearate, Prosolve SMCC (HD90), croscarmellose Sodium, Crospovidone NF, or Avicel PH200. Generally, controlled-release includes sustained but otherwise complete release. A sudden and total release in the large intestine at a desired and appointed time or a release in the intestines such as through the use of an enteric coating are both considered controlled-release. Controlled-release can occur at a predetermined time or in a predetermined place within the digestive tract. It is not meant to include a passive, uncontrolled process as in swallowing a normal tablet. Examples include, but are not limited to, those described in U.S. Pat. Nos. 3,845,770; 3,916,899; 3,536,809; 3,598,123; 4,008,719; 5,674,533; 5,059,595; 5,591,767; 5,120,548; 5,073,543; 5,639,476; 5,354,556; 5,733,556; 5,871,776; 5,902,632; and 5,837,284 each of which is incorporated herein by reference in its entirety.

In an embodiment a controlled release dosage form begins its release and continues that release over an extended period of time. Release can occur beginning almost immediately or can be sustained. Release can be constant, can increase or decrease over time, can be pulsed, can be continuous or intermittent, and the like. Generally, however, the release of at least one pharmaceutically active agent from a controlled-release dosage form will exceed the amount of time of release of the drug taken as a normal, passive release tablet. Thus, for example, while all of at least one pharmaceutically active agent of an uncoated aspirin tablet should be released within, for example, four hours, a controlled-release dosage form could release a smaller amount of aspirin over a period of six hours, 12 hours, or even longer. Controlled-release in accordance with the compositions and methods described herein generally means that the release occurs for a period of six hours or more, such as 12 hours or more.

In another embodiment a controlled release dosage refers to the release of an agent, from a composition or dosage form in which the agent is released according to a desired profile over an extended period of time. In an embodiment, controlled-release results in dissolution of an agent within 20-720 minutes after entering the stomach. In another embodiment, controlled-release occurs when there is dissolution of an agent within 20-720 minutes after being swallowed. In another embodiment, controlled-release occurs when there is dissolution of an agent within 20-720 minutes after entering the intestine. In another embodiment, controlled-release results in substantially complete dissolution after at least 1 hour following administration. In another embodiment, controlled-release results in substantially complete dissolution after at least 1 hour following oral administration. For example, controlled-release compositions allow delivery of an agent to a subject in need thereof over an extended period of time according to a predetermined profile. Such release rates can provide therapeutically effective levels of agent for an extended period of time and thereby provide a longer period of pharmacologic or diagnostic response as compared with conventional rapid release dosage forms. Such longer periods of response provide for many inherent benefits that are not achieved with immediate-release dosages. When used in connection with the dissolution profiles discussed herein, the term “controlled-release” refers to wherein all or less than all of the total amount of a dosage form, made according to methods and compositions described herein, delivers an active agent over a period of time greater than 1 hour.

In one aspect, controlled-release refers to delayed release of an agent, from a composition or dosage form in which the agent is released according to a desired profile in which the release occurs after a period of time.

When present in a controlled-release oral dosage form, the compositions described herein can be administered at a substantially lower daily dosage level than immediate-release forms.

In an embodiment, the controlled-release layer is capable of releasing 30 to 40% of the one or more active agents (e.g., prebiotic and/or probiotic) contained therein in the stomach of a subject in need thereof in 5 to 10 minutes following oral administration. In another embodiment, the controlled-release layer is capable of releasing 90% of the one or more active agents (e.g., prebiotic and/or probiotic) is released in 40 minutes after oral administration.

In some embodiments, the controlled-release layer comprises one or more excipients, including but not limited to silicified microcrystalline cellulose (e.g., HD90), croscarmellose sodium (AC-Di-Sol), hydroxyl methyl propyl cellulose, magnesium stearate, or stearic acid. In an embodiment, a controlled release formulation weighs between 100 mg to 3 g.

Pharmaceutical carriers or vehicles suitable for administration of the compounds provided herein include all such carriers known to those skilled in the art to be suitable for the particular mode of administration. In addition, the compositions can one or more components that do not impair the desired action, or with components that supplement the desired action, or have another action.

In another embodiment, an effective amount of the prebiotic is formulated in an immediate release form. In this embodiment the immediate-release form can be included in an amount that is effective to shorten the time to its maximum concentration in the blood. By way of example, certain immediate-release pharmaceutical preparations are taught in United States Patent Publication US 2005/0147710A1 entitled, “Powder Compaction and Enrobing,” which is incorporated herein in its entirety by reference.

The dosage forms described herein can also take the form of pharmaceutical particles manufactured by a variety of methods, including but not limited to high-pressure homogenization, wet or dry ball milling, or small particle precipitation (nano spray). Other methods to make a suitable powder formulation are the preparation of a solution of active ingredients and excipients, followed by precipitation, filtration, and pulverization, or followed by removal of the solvent by freeze-drying, followed by pulverization of the powder to the desired particle size.

In a further aspect the dosage form can be an effervescent dosage form. Effervescent means that the dosage form, when mixed with liquid, including water and saliva, evolves a gas. Some effervescent agents (or effervescent couple) evolve gas by means of a chemical reaction which takes place upon exposure of the effervescent disintegration agent to water or to saliva in the mouth. This reaction can be the result of the reaction of a soluble acid source and an alkali monocarbonate or carbonate source. The reaction of these two general compounds produces carbon dioxide gas upon contact with water or saliva. An effervescent couple (or the individual acid and base separately) can be coated with a solvent protective or enteric coating to prevent premature reaction. Such a couple can also be mixed with previously lyophilized particles (such as a prebiotic). The acid sources can be any which are safe for human consumption and can generally include food acids, acid and hydrite antacids such as, for example: citric, tartaric, amalic, fumeric, adipic, and succinics. Carbonate sources include dry solid carbonate and bicarbonate salt such as, preferably, sodium bicarbonate, sodium carbonate, potassium bicarbonate and potassium carbonate, magnesium carbonate and the like. Reactants which evolve oxygen or other gasses and which are safe for human consumption are also included. In an embodiment citric acid and sodium bicarbonate are used.

In another aspect the dosage form can be in a candy form (e.g., matrix), such as a lollipop or lozenge. In an embodiment an effective amount of a prebiotic is dispersed within a candy matrix. In an embodiment the candy matrix comprises one or more sugars (such as dextrose or sucrose). In another embodiment the candy matrix is a sugar-free matrix. The choice of a particular candy matrix is subject to wide variation. Conventional sweeteners such as sucrose can be utilized, or sugar alcohols suitable for use with diabetic patients, such as sorbitol or mannitol can be employed. Other sweeteners, such as the aspartame, can also be easily incorporated into a composition in accordance with compositions described herein. The candy base can be very soft and fast dissolving, or can be hard and slower dissolving. Various forms will have advantages in different situations.

A candy mass composition comprising an effective amount of the prebiotic can be orally administered to a subject in need thereof so that an effective amount of the prebiotic will be released into the subject's mouth as the candy mass dissolves and is swallowed. A subject in need thereof includes a human adult or child.

In an embodiment a candy mass is prepared that comprises one or more layers which can comprise different amounts or rates of dissolution of the prebiotic. In an embodiment a multilayer candy mass (such as a lollipop) comprises an outer layer with a concentration of the prebiotic differing from that of one or more inner layers. Such a drug delivery system has a variety of applications.

The choices of matrix and the concentration of the drug in the matrix can be important factors with respect to the rate of drug uptake. A matrix that dissolves quickly can deliver drug into the subject's mouth for absorption more quickly than a matrix that is slow to dissolve. Similarly, a candy matrix that contains the prebiotic in a high concentration can release more of the prebiotic in a given period of time than a candy having a low concentration. In an embodiment a candy matrix such as one disclosed in U.S. Pat. No. 4,671,953 or US Application Publication No. 2004/0213828 (which are herein incorporated by reference in their entirety) is used to deliver the prebiotic.

The dosage forms described herein can also take the form of pharmaceutical particles manufactured by a variety of methods, including but not limited to high-pressure homogenization, wet or dry ball milling, or small particle precipitation (e.g., nGimat's NanoSpray). Other methods useful to make a suitable powder formulation are the preparation of a solution of active ingredients and excipients, followed by precipitation, filtration, and pulverization, or followed by removal of the solvent by freeze-drying, followed by pulverization of the powder to the desired particle size. In an embodiment the pharmaceutical particles have a final size of 3-1000 μM, such as at most 3, 4, 5, 6, 7, 8, 9, 10, 20, 30, 40, 50, 60, 70, 80, 90, 100, 150, 200, 250, 300, 350, 400, 450, 500, 550, 600, 650, 700, 750, 800, 850, 900, 950, 1000 μM. In another embodiment the pharmaceutical particles have a final size of 10-500 μM. In another embodiment the pharmaceutical particles have a final size of 50-600 μM. In another embodiment the pharmaceutical particles have a final size of 100-800 μM.

In an embodiment an oral dosage form (such as a powder, tablet, or capsule) is provided comprising a prebiotic composition comprising 0.7 g of FOS, GOS, or other FOS, GOS, or other appropriate polysaccharide, 0.2 g of lactose, 0.01 g of glucose, 0.01 g of galactose, 0.1-0.2 g of a binder, 0.1-0.2 g of a dispersant, 0.1-0.2 g of a solubilizer, wherein the FOS, GOS, or other FOS, GOS, or other appropriate polysaccharide are composed of 1-25% disaccharides, 1-25% trisaccharides, 1-25% tetrasaccharides, and 1-25% pentasaccharides. The oral dosage form can be in the form of a powder, capsule, or tablet. Suitable amounts of binders, dispersants, and solubilizers are known in the art for preparation of oral tablets or capsules.

In another embodiment an oral dosage form (such as a powder, tablet or capsule) is provided comprising a prebiotic composition comprising 1-99.9% by weight of FOS, GOS, or other FOS, GOS, or other appropriate polysaccharide 0.5-20% by weight of lactose, 0.1-2% by weight of glucose, 0.1-2% by weight of galactose, 0.05-2% by weight of a binder, 0.05-2% by weight of a dispersant, 0.05-2% by weight of a solubilizer, wherein the FOS, GOS, or other FOS, GOS, or other appropriate polysaccharide are composed of 1-25% by weight disaccharides, 1-25% by weight trisaccharides, 1-25% by weight tetrasaccharides, and 1-25% by weight pentasaccharides.

In another embodiment an oral dosage form (such as a powder, tablet, or capsule) is provided comprising a prebiotic composition comprising 1, 10, 20, 30, 40, 50, 60, 70, 80, 90, 95, 99.5, 100% by weight of FOS, GOS, or other FOS, GOS, or other appropriate polysaccharide 0, 5, 10, 15, or 20% by weight of lactose, 0.1, 0.5, 1, or 2% by weight of glucose, 0.1, 0.5, 1, or 2% by weight of galactose, 0.05, 0.1, 0.5, 1, or 2% by weight of a binder, 0.05, 0.1, 0.5, 1, or 2% by weight of a dispersant, 0.05, 0.1, 0.5, 1, or 2% by weight of a solubilizer, wherein the FOS, GOS, or other FOS, GOS, or other appropriate polysaccharide are composed of 1, 5, 10, 15, 20, or 25% by weight disaccharides, 1, 5, 10, 15, 20, or 25% by weight trisaccharides, 1, 5, 10, 15, 20, or 25% by weight tetrasaccharides, and 1, 5, 10, 15, 20, or 25% by weight pentasaccharides.

In another embodiment, an oral dosage form is provided comprising a prebiotic composition, wherein the oral dosage form is a syrup. The syrup can comprise 1%, 5%, 10%, 15%, 20%, 25%, 30%, 35%, 40%, 45%, 50%, 55%, 60%, 65%, 70%, 75%, 80%, or 85% solid. The syrup can comprise 15%, 20%, 25%, 30%, 35%, 40%, 45%, or 50% liquid, for example, water. The solid can comprise a prebiotic composition. The solid can be, for example, 1-96%, 10-96%, 20-96%, 30-96%, 40-96%, 50-96%, 60-96%, 70-96%, 80-96%, or 90-96% prebiotic composition. The solid can be, for example, 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, or 96% prebiotic composition. In an embodiment a prebiotic composition comprises FOS, GOS, or other FOS, GOS, or other appropriate polysaccharide. In another embodiment a prebiotic composition comprises FOS, GOS, or other FOS, GOS, or other appropriate polysaccharide and another prebiotic. In another embodiment a prebiotic composition comprises FOS, GOS or other and inulin or GOS and FOS.

In an embodiment, the softgel capsule is 0.25 mL, 0.5 mL, 1.0 mL, 1.25 mL, 1.5 mL, 1.75 mL, or 2.0 mL. In another embodiment, a softgel capsule comprises 0.1 g to 2.0 g of prebiotic composition. In another embodiment, a softgel capsule comprises 0.1, 0.2, 0.3, 0.4, 0.5, 0.6, 0.7, 0.8, 0.9, 1.0, 1.1, 1.2, 1.3, 1.4, 1.5, 1.6, 1.7, 1.8, 1.9, or 2.0 g of a prebiotic composition. In an embodiment the prebiotic composition comprises FOS, GOS, or other FOS, GOS, or other appropriate polysaccharide. In another embodiment the prebiotic composition consists essentially of FOS, GOS, or other FOS, GOS, or other appropriate polysaccharide. In another embodiment, a softgel capsule comprises FOS, GOS, or other FOS, GOS, or other appropriate polysaccharide and inulin or FOS.

In another embodiment, the prebiotic composition is delivered in a gelatin capsule containing an amount of FOS, GOS, or other FOS, GOS, or other appropriate polysaccharide within the ranges listed in Table 2. In another embodiment, the number of pills taken per day is within the ranges listed in Table 2.

TABLE 2
Exemplary GOS Dosing Units
Exemplary GOS Composition
Dosages in Gel Caps

| | GOS/Pill | # pills |
|---|---|---|
| Size | (g) | per day |
| 000 | 1-2 | 1-15 |
| 00 | 0.6-1.5 | 1-25 |
| 0 | 0.4-1.1 | 1-38 |
| 1 | 0.3-0.8 | 1-50 |
| 2 | 0.25-0.6 | 1-60 |
| 3 | 0.2-0.5 | 1-75 |
| 4 | 0.14-0.3 | 1-107 |

In another embodiment, a prebiotic composition is provided that does not contain a preservative. In another embodiment, a prebiotic composition is provided that does not contain an antioxidant. In another embodiment, a prebiotic composition is provided that does not contain a preservative or an antioxidant. In an embodiment a prebiotic composition comprising FOS, GOS, or other FOS, GOS, or other appropriate polysaccharide does not contain a preservative or an antioxidant.

In another embodiment, a prebiotic composition is formulated as a viscous fluid. In another embodiment, a prebiotic composition is formulated such that its water content is low enough that it does not support microbial growth. In an embodiment, this composition is an intermediate-moisture food, with a water activity between 0.6 and 0.85; in another embodiment this composition is a low-moisture food, with a water activity less than 0.6. Low-moisture foods limit microbial growth significantly and can be produced by one of ordinary skill in the art. For example, these products could be produced similarly to a liquid-centered cough drop. In another embodiment, a prebiotic composition is formulated as a viscous fluid without a preservative in a gel capsule. In another embodiment, a prebiotic composition comprising FOS, GOS, or other FOS, GOS, or other appropriate polysaccharide is a viscous fluid. In another embodiment, a prebiotic composition comprises a high percentage of FOS, GOS, or other FOS, GOS, or other appropriate polysaccharide that does not support microbial growth. In another embodiment, the prebiotic composition comprises FOS, GOS, or other FOS, GOS, or other appropriate polysaccharide and inulin or FOS.

In another embodiment, an oral dosage form is provided comprising a prebiotic composition, wherein the oral dosage form is a softgel. In an embodiment the softgel comprises a syrup. In an embodiment the syrup comprises a prebiotic composition. In an embodiment the prebiotic composition comprises FOS, GOS, or other FOS, GOS, or other appropriate polysaccharide. In another embodiment the prebiotic composition comprises more than 80% FOS, GOS, or other FOS, GOS, or other appropriate polysaccharide. In another embodiment the prebiotic composition comprises between 80-99.9% FOS, GOS, or other. In another embodiment the prebiotic composition comprises more than 80% FOS, GOS, or other FOS, GOS, or other appropriate polysaccharide. In another embodiment the prebiotic composition comprises 80, 81, 82, 83, 84, 85, 86, 87, 88, 89, 90, 91, 92, 93, 94, 95, 96, 97, 98, 99, or 99.9% FOS, GOS, or other FOS, GOS, or other appropriate polysaccharide.

In an embodiment a FOS, GOS, or other FOS, GOS, or other appropriate polysaccharide composition is formulated for delivery in a soft gel capsule. In an embodiment a FOS, GOS, or other FOS, GOS, or other appropriate polysaccharide composition formulated for delivery in a soft gel capsule is a high percentage FOS, GOS, or other FOS, GOS, or other appropriate polysaccharide composition, such as a 90-100% FOS, GOS, or other FOS, GOS, or other appropriate polysaccharide composition (e.g., 90, 91, 92, 93, 94, 95, 96, 97, 98, 99 or 100% FOS, GOS, or other FOS, GOS, or other appropriate polysaccharide composition by weight). In another embodiment a FOS, GOS, or other FOS, GOS, or other appropriate polysaccharide composition formulated for delivery in a soft gel capsule comprises 95% FOS, GOS, or other FOS, GOS, or other appropriate polysaccharide. In another embodiment a FOS, GOS, or other FOS, GOS, or other appropriate polysaccharide composition formulated for delivery in a soft gel capsule comprises 96% FOS, GOS, or other FOS, GOS, or other appropriate polysaccharide. In another embodiment, the FOS, GOS, or other FOS, GOS, or other appropriate polysaccharide composition is formulated such that its water content is low enough that it does not support microbial growth. In another embodiment, the FOS, GOS, or other FOS, GOS, or other appropriate polysaccharide composition is formulated as a viscous fluid without a preservative in a gel capsule. In another embodiment, the FOS, GOS, or other FOS, GOS, or other appropriate polysaccharide composition is formulated as a viscous fluid without an antioxidant in a gel capsule. In another embodiment the soft gel capsule comprises 0.1-2 g of a FOS, GOS, or other FOS, GOS, or other appropriate polysaccharide composition.

In another embodiment a prebiotic composition can be formulated as described, in U.S. Pat. No. 6,750,331, which is herein incorporated by reference in its entirety. A prebiotic composition can be formulated to comprise an oligosaccharide, a foaming component, a water-insoluble dietary fiber (e.g., cellulose or lignin), or a neutralizing component. In an embodiment a prebiotic composition can be in the form of a chewable tablet.

In an embodiment a foaming component can be at least one member selected from the group consisting of sodium hydrogencarbonate, sodium carbonate, and calcium carbonate. In an embodiment a neutralizing component can be at least one member selected from the group consisting of citric acid, L-tartaric acid, fumaric acid, L-ascorbic acid, DL-malic acid, acetic acid, lactic acid, and anhydrous citric acid. In an embodiment a water-insoluble dietary fiber can be at least one member selected from the group consisting of crystalline cellulose, wheat bran, oat bran, cone fiber, soy fiber, and beet fiber. The formulation can contain a sucrose fatty acid ester, powder sugar, fruit juice powder, and/or flavoring material.

Formulations of the provided invention can include additive components selected from various known additives. Such additives include, for example, saccharides (excluding oligosaccharides), sugar alcohols, sweeteners and like excipients, binders, disintegrators, lubricants, thickeners, surfactants, electrolytes, flavorings, coloring agents, pH modifiers, fluidity improvers, and the like. Specific examples of the additives include wheat starch, potato starch, corn starch, dextrin and like starches; sucrose, glucose, fructose, maltose, xylose, lactose and like saccharides (excluding oligosaccharides); sorbitol, mannitol, maltitol, xylitol and like sugar alcohols; calcium phosphate, calcium sulfate and like excipients; starch, saccharides, gelatin, gum arabic, dextrin, methyl cellulose, polyvinylpyrrolidone, polyvinyl alcohol, hydroxypropylcellulose, xanthan gum, pectin, gum tragacanth, casein, alginic acid and like binders and thickeners; leucine, isoleucine, L-valine, sugar esters, hardened oils, stearic acid, magnesium stearate, talc, macrogols and like lubricants; CMC, CMC-Na, CMC-Ca and like disintegrators; polysorbate, lecithin and like surfactants; aspartame, alitame and like dipeptides; silicon dioxide and like fluidity improvers; and stevia, saccharin, and like sweeteners. The amounts of these additives can be properly selected based on their relation to other components and properties of the preparation, production method, etc.

In an embodiment, a FOS, GOS, or other FOS, GOS, or other appropriate polysaccharide composition is a chewable oral dosage formulation. In an embodiment the chewable formulation can comprises between 1-99.9% FOS, GOS, or other FOS, GOS, or other appropriate polysaccharide. In an embodiment, a FOS, GOS, or other FOS, GOS, or other appropriate polysaccharide composition comprises 80% FOS, GOS, or other FOS, GOS, or other appropriate polysaccharide 5% L-ascorbic acid, 2% anhydrous citric acid, 3% sodium hydrogencarbonate, 3% calcium carbonate, 2% sucrose fatty acid, 3% fruit juice powder, and 2% potassium carbonate.

In another embodiment, a FOS, GOS, or other FOS, GOS, or other appropriate polysaccharide composition comprises 85% FOS, GOS, or other FOS, GOS, or other appropriate polysaccharide, 5% L-ascorbic acid, 3% sodium hydrogencarbonate, 2% sodium carbonate, 2% sucrose fatty acid ester, 2% fruit juice powder, and 1% potassium carbonate.

In another embodiment, a FOS, GOS, or other FOS, GOS, or other appropriate polysaccharide composition comprises 90% FOS, GOS, or other FOS, GOS, or other appropriate polysaccharide, 2% L-ascorbic acid, 1% anhydrous citric acid, 2% sodium hydrogencarbonate, 2% sodium carbonate, 2% sucrose fatty acid ester, and 1% potassium carbonate.

In another embodiment, a FOS, GOS, or other FOS, GOS, or other appropriate polysaccharide composition comprises 95% FOS, GOS, or other FOS, GOS, or other appropriate polysaccharide, 2% L-ascorbic acid, 1% sodium hydrogencarbonate, and 2% fruit juice powder. In another embodiment, a FOS, GOS, or other FOS, GOS, or other appropriate polysaccharide composition comprises 95% FOS, GOS, or other FOS, GOS, or other appropriate polysaccharide and 5% of L-ascorbic acid, anhydrous citric acid, sodium hydrogencarbonate, calcium carbonate, sucrose fatty acid, fruit juice powder, or potassium carbonate.

In another embodiment, a FOS, GOS, or other FOS, GOS, or other appropriate polysaccharide composition comprises 95% FOS, GOS, or other FOS, GOS, or other appropriate polysaccharide and 5% of L-ascorbic acid, anhydrous citric acid, sodium hydrogencarbonate, calcium carbonate, sucrose fatty acid, fruit juice powder, and potassium carbonate.

### Combination Therapy

In some embodiments, the compositions of the present invention can be used in conjunction with traditional treatments for a musculoskeletal disorder, such as an anti-osteoporosis or osteopenia therapy. In some embodiments, the present invention is administered together with at least one other agent. In some embodiments, the present invention is administered before the at least one other agent. In other embodiments, the present invention is administered after cessation of another therapy. The therapy includes, but is not limited to, approved therapies for osteoporosis, osteopenia, Paget's disease, stunting, osteoarthritis, osteomyelitis, delayed or on-union fractures, or any combination of the foregoing.

Some therapies for osteoporosis or osteopenia that are known in the art include: estrogen, estrogen agonists, estrogen antagonists, and bisphosphonates. One of skill in the art would understand that the present invention may be used to supplement, increase efficacy of, or otherwise improve upon any of a number of known therapies for osteoporosis or osteopenia.

### Medical Foods

An alternate embodiment of the present invention is a formulation as a medical food.

The consuming public has come to understand that foods possess more than basic nutrition (protein, carbohydrate, fat, etc). For example, 95% of consumers agree that “certain foods have health benefits that go beyond basic nutrition and may reduce the risk of disease or other health concerns.” More than 50% of consumers believe that foods can replace the use of drugs. Replacing the use of drugs may have the benefit of reducing the incidence of adverse side effects suffered by patients following a pharmaceutical drug treatment regimen. In fact, medical foods are assumed to be generally safe, as people have historically consumed these foods safely in non-medical contexts.

The compositions of the invention may be administered under the supervision of a medical specialist, or may be self-administered. Medical foods could take the form of nutritional shakes or other liquids or meal replacements. Medical foods of the present invention could also take the form of a powder capable of being consumed upon addition to suitable food or liquid.

A medical food formulation of the present invention could confer benefits of a synthetic composition of microbes isolated from nutritionally beneficial plants, as well as the benefits of prebiotics, or other nutritionally beneficial inclusions, but not consumed to obtain nutrition from them but rather to provide a metabolic function different than a foodstuff. For example, medical foods of the invention may also include at least one vitamin, or vitamin precursor. Preferred vitamins possess antioxidant properties and include vitamins A, C and E. and/or their biochemical precursors. Another embodiment of the medical foods of the invention also includes at least one trace element, preferably selected from the group consisting of zine, manganese and selenium. Medical foods of the invention also may include at least one additional antioxidant selected from the group consisting of carotenoids, N-acetylcysteine and L-glutamine. It is known to those of skill in the art how to construct medical foods containing these elements.

Medical foods of the present invention would include effective doses of microbes deemed useful for the indication and effective doses of any vitamin, prebiotic, or other beneficial additive not consumed to obtain nutrition but to add a therapeutic benefit mediated by the production of SCFA or other immuno-stimulant molecules when passing through the GI tract.

Typically, the dietary supplements and medical foods of the present invention are consumed at least once daily, and preferably administered two times per day, preferably once in the morning and once in the afternoon. A typical treatment regime for the dietary supplements or medical foods will continue for four to eight weeks. Depending on such factors as the medical condition being treated and the response of the patient, the treatment regime may be extended. A medical food of the present invention will typically be consumed in two servings per day as either a meal replacement or as a snack between meals.

Anyone perceived to be at risk from a musculoskeletal disorder, including, or already suffering from any of the foregoing, can potentially benefit from ingesting the compositions of the invention. According to the invention it is believed to be possible to effectively ameliorate symptoms and conditions associated with musculoskeletal disorders with natural compounds, which do not show any severe side effects. Furthermore, the present methods are expected to be well-tolerated, for example without causing any discomfort or nausea, and simple to apply.

### EXAMPLES

Below are examples of specific embodiments for carrying out the present invention. The examples are offered for illustrative purposes only, and are not intended to limit the scope of the present invention in any way. Efforts have been made to ensure accuracy with respect to numbers used (e.g., amounts, temperatures, etc.), but some experimental error and deviation should, of course, be allowed for.

### Example 1: Microbial Preparations and Metagenomic Analyses

A sample set of 15 vegetables typically eaten raw was selected to analyze the microbial communities by whole genome shotgun sequencing and comparison to microbial databases. The 15 fruits and vegetable samples are shown in Table 3 and represent ingredients in typical salads or eaten fresh. The materials were sourced at the point of distribution in supermarkets selling both conventional and organic farmed vegetables, either washed and ready to cat or without washing.

The samples were divided into 50 g portions, thoroughly rinsed with tap water and blended for 30 seconds on phosphate buffer pH 7.4 (PBS) in a household blender. The resulting slurry was strained by serial use of a coarse and then a fine household sieve followed by filtration through a 40 mm sieve. The cell suspension containing the plant microbiota, chloroplasts and plant cell debris was centrifuged at slow speed for removing plant material and the resulting supernatant centrifuged at high speed to pellet microbial cells. The pellet resuspended in a buffer containing a proprietary plant cell lysis buffer consisting of chelating agents such as EDTA or Versetene EDTA-based chelating agents to remove divalent ions and a suitable non-ionic detergent such as Tween-20, Tween 80, Triton X, and washed then with PBS. For sample #12 (broccoli) the cell pellet was washed and a fraction of the biomass separated and only the top part of the pellet collected. This was deemed “broccoli juice” for analyses. The resulting microbiota prep was inspected under fluorescence microscopy with DNA stains to visualize plant and microbial cells based on cell size and DNA structure (nuclei for plants) and selected for DNA isolation based on a minimum ratio of 9:1 microbe to plant cells. The DNA isolation was based on the method reported by Marmur (1961), or using commercial DNA extraction kits based on magnetic beads such as Thermo Charge Switch resulting in a quality suitable for DNA library prep and free of PCR inhibitors.

The DNA was used to construct a single read 150 base pair libraries and a total of 26 million reads sequenced per sample according to the standard methods done by CosmosID (www.cosmosid.com) for samples #1 to #12 or 300 base pair-end libraries and sequenced in an Illumina NextSeq instrument covering 4 Gigabases per sample for samples #13 to #15. The unassembled reads were then mapped to the CosmosID for first 12 samples or OneCodex for the last 3 samples databases containing 36,000 reference bacterial genomes covering representative members from diverse taxa. The mapped reads were tabulated and represented using a “sunburst” plot to display the relative abundance for each genome identified corresponding to that bacterial strain and normalized to the total of identified reads for each sample. In addition, phylogenetic trees were constructed based on the classification for each genome in the database with a curated review. There are genomes that have not been updated in the taxonomic classifier and therefore reported as unclassified here but it does not reflect a true lack of clear taxonomic position, it reflects only the need for manual curation and updating of those genomes in the taxonomic classifier tool. Lastly, samples 16 to 21 were analyzed using Kraken2 taxonomic sequence classification approach (Wood and Salzberg, 2014). The unassembled reads were filtered out by mapping the reads to each plant host genome sequences if available. Taxonomic labels were assigned to each sequencing read by Kraken2 according to the standard Kraken2 database that includes complete RefSeq genome sequences (O'Leary et al. 2016). Then, the abundance of species in each metagenomic sample was estimated using Bracken (Lu et al. 2017). The relative abundances were presented in pie chart at each taxonomic level.

In addition to the shotgun metagenomics survey, relevant microbes were isolated from fruits and vegetables listed in Table 3 using potato dextrose agar, nutrient agar or MRS agar and their genomes sequenced to cover 50× and analyzed their metabolic potential by using genome-wide models. For example, a yeast isolated from blueberries was sequenced and its genome showed identity to Aureobasidium subglaciale assembled in contigs with an N50 of 71 Kb and annotated to code for 10, 908 genes. Similarly, bacterial genomes from the same sample were sequenced and annotated for strains with high identity to Pseudomonas and Rahnella.

TABLE 3
Samples analyzed.

| Sample number | sample description |
|---|---|
| 1 | Chard |
| 2 | Red cabbage |
| 3 | Romaine romaine |
| 4 | Celery |
| 5 | Butterhead lettuce |
| 6 | Baby spinach |
| 7 | Crisp green gem lettuce |
| 8 | Red oak leaf lettuce |
| 9 | Green oak leaf lettuce |
| 10 | Cherry tomato |
| 11 | Crisp red gem lettuce |
| 12 | Broccoli juice |
| 13 | Broccoli head |
| 14 | Blueberries |
| 15 | Pickled olives |
| 16 | Gingseng |
| 17 | Blackberries |
| 18 | Squash gourd |
| 19 | Broccolini |
| 20 | Fermented cabbage |
| 21 | Fermented pepper paste |

### Results

For most samples, bacterial abundances of fresh material contain 10\hat{ }4 to 10\hat{ }8 microbes per gram of vegetable as estimated by direct microscopy counts or viable counts. Diverse cell morphologies were observed including rods, elongated rods, cocci and fungal hyphae. Microorganisms were purified from host cells, DNA was isolated and sequenced using a shotgun approach mapping reads to 35,000 bacterial genomes applying a k-mer method using Cosmos ID (https://www.cosmosid.com/) . . . . All samples were dominated by gamma proteobacteria, primarily Pseudomonadacea, presumably largely endophytes as some samples were triple washed before packaging. Pseudomonas cluster was the dominant genera for several samples with 10-90% of the bacterial relative abundance detected per sample and mapped to a total of 27 different genomes indicating it is a diverse group. A second relevant bacterial strain identified was Duganella zoogloeoides ATCC 25935 as it was present in almost all the samples ranging from 1-6% of the bacterial relative abundance detected per sample or can reach 29% of the bacterial relative abundance detected per sample in organic romaine. Red cabbage was identified to contain a relatively large proportion of lactic acid bacteria as it showed 22% Lactobacillus crispatus, a species commercialized as probiotic and recognized relevant in vaginal healthy microbial community. Another vegetable containing lactic acid bacteria was red oak leaf lettuce containing 1.5% of the bacterial relative abundance detected per sample Lactobacillus reuteri. Other bacterial species recognized as probiotics included Bacillus, Bacteroidetes, Propionibacterium and Streptococcus. A large proportion of the abundant taxa in most samples was associated with plant microbiota and members recognized to act as biocontrol agents against fungal diseases or growth promoting agents such as Pseudomonas fluorescens. The aggregated list of unique bacteria detected by the k-mer method is 287 (Table 4).

Blueberries contain a mixture of bacteria and fungi dominated by Pseudomonas and Propionibacterium but the yeast Aureobasidium was identified as a relevant member of the community. A lesser abundant bacterial species was Rahnella. Pickled olives are highly enriched in lactic acid bacteria after being pickled in brine allowing the endogenous probiotic populations to flourish by acidifying the environment and eliminating most of the acid-sensitive microbes including bacteria and fungi. This resulted in a large amount of Lactobacillus species and Pediococcus recognized as probiotics and related to osteoporosis treatment. Other fermented samples included fermented cabbage and chili pepper paste. Fermented cabbage contained Pediococcus pentosaceus as well as dominant gamma proteobacteria. Fermented chili pepper paste enriched for Lactobacillus with 31% of the bacterial population but also Leuconostoc mesenteroides and Pediococcus pentosaceus were enriched. One unexpected sample containing lactic acid bacteria was squash gourd showing 59% Lactococcus but also Leuconostoc was present at 3.5% of the bacterial population. In addition to the bacterial populations, some samples also contained yeast not shown in Kraken2 plots from which Pichia was isolated, such as fermented chili pepper paste.

The shotgun sequencing method allows for the analysis of the metagenome including genes coding for metabolic reactions involved in the assimilation of nutrient, fermentative processes to produce short chain fatty acids, flavonoids and other relevant molecules in human nutrition.

TABLE 4
Bacteria identified in a 15 sample survey identified by whole genome matching to
reference genomes. The fruits and vegetables were selected based on their recognition
as part of the whole food plant-based diet and some antidiabetic and obesogenic
properties. There is general recognition of microbes in these vegetables relevant
for plant health but not previously recognized for their use in human health.

| Strains identified by k-mer based on entire genome | Strain number | Collection |
|---|---|---|
| Acinetobacter baumannii | — | |
| Acinetobacter soli | — | |
| Acinetobacter 41764 Branch | — | |
| Acinetobacter 41930 Branch | — | |
| Acinetobacter 41981 Branch | — | |
| Acinetobacter 41982 Branch | — | |
| Acinetobacter baumannii 348935 | — | |
| Acinetobacter baumannii 40298 Branch | — | |
| Acinetobacter beijerinckii 41969 Branch | — | |
| Acinetobacter beijerinckii CIP 110307 | CIP 110307 | WFCC |
| Acinetobacter bohemicus ANC 3994 | — | |
| Acinetobacter guillouiae 41985 Branch | — | |
| Acinetobacter guillouiae 41986 Branch | — | |
| Acinetobacter gyllenbergii 41690 Branch | — | |
| Acinetobacter haemolyticus TG19602 | — | |
| Acinetobacter harbinensis strain HITLi 7 | — | |
| Acinetobacter johnsonii 41886 Branch | — | |
| Acinetobacter johnsonii ANC 3681 | — | |
| Acinetobacter junii 41994 Branch | — | |
| Acinetobacter lwoffii WJ10621 | — | |
| Acinetobacter sp 41945 Branch | — | |
| Acinetobacter sp 41674 Branch | — | |
| Acinetobacter sp 41698 Branch | — | |
| Acinetobacter sp ETR1 | — | |
| Acinetobacter sp NIPH 298 | — | |
| Acinetobacter tandoii 41859 Branch | — | |
| Acinetobacter tjernbergiae 41962 Branch | — | |
| Acinetobacter towneri 41848 Branch | — | |
| Acinetobacter venetianus VE C3 | — | |
| Actinobacterium LLX17 | — | |
| Aeromonas bestiarum strain CECT 4227 | CECT 4227 | CECT |
| Aeromonas caviae strain CECT 4221 | CECT 4221 | CECT |
| Aeromonas hydrophila 4AK4 | — | |
| Aeromonas media 37528 Branch | — | |
| Aeromonas media strain ARB 37524 Branch | — | |
| Aeromonas salmonicida subsp 37538 Branch | — | |
| Aeromonas sp ZOR0002 | — | |
| Agrobacterium 22298 Branch | — | |
| Agrobacterium 22301 Branch | — | |
| Agrobacterium 22313 Branch | — | |
| Agrobacterium 22314 Branch | — | |
| Agrobacterium sp ATCC 31749 | ATCC 31749 | ATCC |
| Agrobacterium tumefaciens 22306 Branch | | |
| Agrobacterium tumefaciens strain MEJ076 | — | |
| Agrobacterium tumefaciens strain S2 | — | |
| Alkanindiges illinoisensis DSM 15370 | DSM 15370 | WFCC |
| alpha proteobacterium L41A | — | |
| Arthrobacter 20515 Branch | — | |
| Arthrobacter arilaitensis Re117 | — | |
| Arthrobacter chlorophenolicus A6 | — | |
| Arthrobacter nicotinovorans 20547 Branch | — | |
| Arthrobacter phenanthrenivorans Sphe3 | — | |
| Arthrobacter sp 20511 Branch | — | |
| Arthrobacter sp PAO19 | — | |
| Arthrobacter sp W1 | — | |
| Aureimonas sp. Leaf427 | — | |
| Aureobasidium pullulans | — | |
| Bacillaceae Family 24 4101 12691 Branch | — | |
| Bacillus sp. LL01 | — | |
| Bacillus 12637 Branch | — | |
| Bacillus aerophilus strain C772 | — | |
| Bacillus thuringiensis serovar 12940 Branch | — | |
| Brevundimonas nasdae strain TPW30 | — | |
| Brevundimonas sp 23867 Branch | — | |
| Brevundimonas sp EAKA | — | |
| Buchnera aphidicola str 28655 Branch | — | |
| Burkholderiales Order 15 6136 Node 25777 | — | |
| Buttiauxella agrestis 35837 Branch | — | |
| Candidatus Burkholderia verschuerenii | — | |
| Carnobacterium 5833 Branch | — | |
| Carnobacterium maltaromaticum ATCC 35586 | ATCC 35586 | ATCC |
| Chryseobacterium 285 Branch | — | |
| Chryseobacterium daeguense DSM 19388 | DSM 19388 | WFCC |
| Chryseobacterium formosense | — | |
| Chryseobacterium sp YR005 | — | |
| Clavibacter 20772 Branch | — | |
| Clostridium diolis DSM 15410 | DSM 15410 | WFCC |
| Comamonas sp B 9 | — | |
| Curtobacterium flaccumfaciens 20762 Branch | — | |
| Curtobacterium flaccumfaciens UCD AKU | — | |
| Curtobacterium sp UNCCL17 | — | |
| Deinococcus aquatilis DSM 23025 | DSM 23025 | WFCC |
| Debaromyces hansenii | ATCC 36239 | ATCC |
| Duganella zoogloeoides | ATCC 25935 | |
| Dyadobacter 575 Branch | — | |
| Elizabethkingia anophelis | — | |
| Empedobacter falsenii strain 282 | — | |
| Enterobacter sp 638 | — | |
| Enterobacteriaceae Family 9 3608 Node 35891 | — | |
| Enterobacteriaceae Family 9 593 Node 36513 | — | |
| Epilithonimonas lactis | — | |
| Epilithonimonas tenax DSM 16811 | DSM 16811 | WFCC |
| Erwinia 35491 Branch | — | |
| Erwinia amylovora 35816 Branch | — | |
| Erwinia pyrifoliae 35813 Branch | — | |
| Erwinia tasmaniensis Et1 99 | DSM 17950 | WFCC |
| Escherichia coli ISC11 | — | |
| Exiguobacterium 13246 Branch | — | |
| Exiguobacterium 13260 Branch | — | |
| Exiguobacterium sibiricum 255 15 | DSM 17290 | WFCC |
| Exiguobacterium sp 13263 Branch | — | |
| Exiguobacterium undae 13250 Branch | — | |
| Exiguobacterium undae DSM 14481 | DSM 14481 | WFCC |
| Flavobacterium 237 Branch | — | |
| Flavobacterium aquatile LMG 4008 | LMG 4008 | WFCC |
| Flavobacterium chungangense LMG 26729 | LMG 26729 | WFCC |
| Flavobacterium daejeonense DSM 17708 | DSM 17708 | WFCC |
| Flavobacterium hibernum strain DSM 12611 | DSM 12611 | WFCC |
| Flavobacterium hydatis | — | |
| Flavobacterium johnsoniae UW101 | ATCC 17061D-5 | ATCC |
| Flavobacterium reichenbachii | — | |
| Flavobacterium soli DSM 19725 | DSM 19725 | WFCC |
| Flavobacterium sp 238 Branch | — | |
| Flavobacterium sp EM1321 | — | |
| Flavobacterium sp MEB061 | — | |
| Hanseniaspora uvarum | ATCC 18859 | |
| Hanseniaspora occidentalis | ATCC 32053 | |
| Herminiimonas arsenicoxydans | — | |
| Hymenobacter swuensis DY53 | — | |
| Janthinobacterium 25694 Branch | — | |
| Janthinobacterium agaricidamnosum | DSM 9628 | WFCC |
| NBRC 102515 | | |
| Janthinobacterium lividum strain RIT308 | — | |
| Janthinobacterium sp RA13 | — | |
| Kocuria 20614 Branch | — | |
| Kocuria rhizophila 20623 Branch | — | |
| Lactobacillus acetotolerans | — | |
| Lactobacillus brevis | — | |
| Lactobacillus buchneri | — | |
| Lactobacillus futsaii | — | |
| Lactobacillus kefiranofaciens | — | |
| Lactobacillus panis | — | |
| Lactobacillus parafarraginis | — | |
| Lactobacillus plantarum | — | |
| Lactobacillus rapi | — | |
| Lactobacillus crispatus 5565 Branch | — | |
| Lactobacillus plantarum WJL | — | |
| Lactobacillus reuteri 5515 Branch | — | |
| Leuconostoc mesenteroides | ATCC 8293 | |
| Luteibacter sp 9135 | — | |
| Massilia timonae CCUG 45783 | — | |
| Methylobacterium extorquens 23001 Branch | — | |
| Methylobacterium sp 22185 Branch | — | |
| Methylobacterium sp 285MFTsu5 1 | — | |
| Methylobacterium sp 88A | — | |
| Methylotenera versatilis 7 | — | |
| Microbacterium laevaniformans OR221 | — | |
| Microbacterium oleivorans | — | |
| Microbacterium sp MEJ108Y | — | |
| Microbacterium sp UCD TDU | — | |
| Microbacterium testaceum StLB037 | — | |
| Micrococcus luteus strain RIT304 | NCTC 2665 | NCTC |
| Mycobacterium abscessus 19573 Branch | — | |
| Neosartorya fischeri | — | |
| Oxalobacteraceae bacterium AB 14 | — | |
| Paenibacillus sp FSL 28088 Branch | — | |
| Paenibacillus sp FSL H7 689 | — | |
| Pantoea sp. SL1 M5 | — | |
| Pantoea 36041 Branch | — | |
| Pantoea agglomerans strain 4 | — | |
| Pantoea agglomerans strain 4 | — | |
| Pantoea agglomerans strain LMAE 2 | — | |
| Pantoea agglomerans Tx10 | — | |
| Pantoea sp 36061 Branch | — | |
| Pantoea sp MBLJ3 | — | |
| Pantoea sp SL1 M5 | — | |
| Paracoccus sp PAMC 22219 | — | |
| Patulibacter minatonensis DSM 18081 | DSM 18081 | WFCC |
| Pectobacterium carotovorum subsp | — | |
| carotovorum strain 28625 Branch | | |
| Pediococcus ethanolidurans | — | |
| Pediococcus pentosaceus | ATCC 33314 | |
| Pedobacter 611 Branch | — | |
| Pedobacter agri PB92 | — | |
| Pedobacter borealis DSM 19626 | DSM 19626 | WFCC |
| Pedobacter kyungheensis strain KACC 16221 | — | |
| Pedobacter sp R20 19 | — | |
| Periglandula ipomoeae | — | |
| Planomicrobium glaciei CHR43 | — | |
| Propionibacterium acnes | — | |
| Propionibacterium 20955 Branch | — | |
| Propionibacterium acnes 21065 Branch | — | |
| Pseudomonas fluorescens | — | |
| Pseudomonas sp. DSM 29167 | — | |
| Pseudomonas sp. Leaf15 | — | |
| Pseudomonas syringae | — | |
| Pseudomonas 39524 Branch | — | |
| Pseudomonas 39642 Branch | — | |
| Pseudomonas 39733 Branch | — | |
| Pseudomonas 39744 Branch | — | |
| Pseudomonas 39791 Branch | — | |
| Pseudomonas 39821 Branch | — | |
| Pseudomonas 39834 Branch | — | |
| Pseudomonas 39875 Branch | — | |
| Pseudomonas 39880 Branch | — | |
| Pseudomonas 39889 Branch | — | |
| Pseudomonas 39894 Branch | — | |
| Pseudomonas 39913 Branch | — | |
| Pseudomonas 39931 Branch | — | |
| Pseudomonas 39942 Branch | — | |
| Pseudomonas 39979 Branch | — | |
| Pseudomonas 39996 Branch | — | |
| Pseudomonas 40058 Branch | — | |
| Pseudomonas 40185 Branch | — | |
| Pseudomonas abietaniphila strain KF717 | — | |
| Pseudomonas chlororaphis strain EA105 | — | |
| Pseudomonas cremoricolorata DSM 17059 | DSM 17059 | WFCC |
| Pseudomonas entomophila L48 | — | |
| Pseudomonas extremaustralis 14 3 substr 14 3b | — | |
| Pseudomonas fluorescens BBc6R8 | — | |
| Pseudomonas fluorescens BS2 | ATCC 12633 | ATCC |
| Pseudomonas fluorescens EGD AQ6 | — | |
| Pseudomonas fluorescens strain | — | |
| AU 39831 Branch | | |
| Pseudomonas fluorescens strain AU10973 | — | |
| Pseudomonas fluorescens strain AU14440 | — | |
| Pseudomonas fragi B25 | NCTC 10689 | NCTC |
| Pseudomonas frederiksbergensis strain SI8 | — | |
| Pseudomonas fulva strain MEJ086 | — | |
| Pseudomonas fuscovaginae 39768 Branch | — | |
| Pseudomonas gingeri NCPPB 3146 | NCPPB 3146 | NCPPB |
| Pseudomonas lutea | — | |
| Pseudomonas luteola XLDN4 9 | — | |
| Pseudomonas mandelii JR 1 | — | |
| Pseudomonas moraviensis R28 S | — | |
| Pseudomonas mosselii SJ10 | — | |
| Pseudomonas plecoglossicida NB 39639 Branch | — | |
| Pseudomonas poae RE*1 1 14 | — | |
| Pseudomonas pseudoalcaligenes AD6 | — | |
| Pseudomonas psychrophila HA 4 | — | |
| Pseudomonas putida DOT T1E | — | |
| Pseudomonas putida strain KF703 | — | |
| Pseudomonas putida strain MC4 5222 | — | |
| Pseudomonas rhizosphaerae | — | |
| Pseudomonas rhodesiae strain FF9 | — | |
| Pseudomonas sp 39813 Branch | — | |
| Pseudomonas simiae strain 2 36 | — | |
| Pseudomonas simiae strain MEB105 | — | |
| Pseudomonas sp 11 12A | — | |
| Pseudomonas sp 2 922010 | — | |
| Pseudomonas sp CF149 | — | |
| Pseudomonas sp Eur1 9 41 | — | |
| Pseudomonas sp LAMO17WK12 I2 | — | |
| Pseudomonas sp PAMC 25886 | — | |
| Pseudomonas sp PTA1 | — | |
| Pseudomonas sp R62 | — | |
| Pseudomonas sp WCS374 | — | |
| Pseudomonas synxantha BG33R | — | |
| Pseudomonas synxantha BG33R | — | |
| Pseudomonas syringae 39550 Branch | — | |
| Pseudomonas syringae 39596 Branch | — | |
| Pseudomonas syringae 40123 Branch | — | |
| Pseudomonas syringae CC 39499 Branch | — | |
| Pseudomonas syringae pv panici str LMG 2367 | — | |
| Pseudomonas syringae strain mixed | — | |
| Pseudomonas tolaasii 39796 Branch | — | |
| Pseudomonas tolaasii PMS117 | — | |
| Pseudomonas veronii 1YdBTEX2 | — | |
| Pseudomonas viridiflava CC1582 | — | |
| Pseudomonas viridiflava strain LMCA8 | — | |
| Pseudomonas viridiflava TA043 | — | |
| Pseudomonas viridiflava UASWS0038 | — | |
| Rahnella 35969 Branch | — | |
| Rahnella 35970 Branch | — | |
| Rahnella 35971 Branch | — | |
| Rahnella aquatilis HX2 | — | |
| Rahnella sp WP5 | — | |
| Raoultella ornithinolytica | — | |
| Rhizobiales Order 22324 Branch | — | |
| Rhizobium sp YR528 | — | |
| Rhodococcus fascians A76 | — | |
| Rhodococcus sp BS 15 | — | |
| Saccharomyces cerevisiae | — | |
| Sanguibacter keddieii | DSM 10542 | WFCC |
| Serratia fonticola AU 35657 Branch | — | |
| Serratia fonticola AU AP2C | — | |
| Serratia liquefaciens ATCC 27592 | ATCC 27592 | ATCC |
| Serratia sp H 35589 Branch | — | |
| Shewanella 37294 Branch | — | |
| Shewanella baltica 37301 Branch | — | |
| Shewanella baltica 37315 Branch | — | |
| Shewanella baltica OS 37308 Branch | — | |
| Shewanella baltica OS 37312 Branch | — | |
| Shewanella baltica OS185 | — | |
| Shewanella baltica OS223 | — | |
| Shewanella baltica OS678 | — | |
| Shewanella oneidensis MR 1 | — | |
| Shewanella putrefaciens HRCR 6 | — | |
| Shewanella sp W3 18 1 | — | |
| Sphingobacterium sp ML3W | — | |
| Sphingobium japonicum BiD32 | — | |
| Sphingobium xenophagum 24443 Branch | — | |
| Sphingomonas echinoides ATCC 14820 | ATCC 14820 | ATCC |
| Sphingomonas parapaucimobilis NBRC 15100 | ATCC 51231 | ATCC |
| Sphingomonas paucimobilis NBRC 13935 | ATCC 29837 | ATCC |
| Sphingomonas phyllosphaerae 5 2 | — | |
| Sphingomonas sp 23777 Branch | — | |
| Sphingomonas sp STIS6 2 | — | |
| Staphylococcus 6317 Branch | — | |
| Staphylococcus equorum UMC CNS 924 | — | |
| Staphylococcus sp 6275 Branch | — | |
| Staphylococcus sp 6240 Branch | — | |
| Staphylococcus sp OJ82 | — | |
| Staphylococcus xylosus strain LSR 02N | — | |
| Stenotrophomonas 14028 Branch | — | |
| Stenotrophomonas 42816 Branch | — | |
| Stenotrophomonas maltophilia 42817 Branch | — | |
| Stenotrophomonas maltophilia PML168 | — | |
| Stenotrophomonas maltophilia strain ZBG7B | — | |
| Stenotrophomonas rhizophila | — | |
| Stenotrophomonas sp RIT309 | — | |
| Streptococcus gallofyticus | — | |
| subsp gallofyticus TX20005 | | |
| Streptococcus infantarius | — | |
| subsp infantarius 2242 Branch | | |
| Streptococcus infantarius | ATCC BAA 102 | ATCC |
| subsp infantarius ATCC BAA 102 | | |
| Streptococcus macedonicus ACA DC 198 | ATCC BAA-249 | ATCC |
| Streptomyces olindensis | — | |
| Variovorax paradoxus 110B | — | |
| Variovorax paradoxus ZNC0006 | — | |
| Variovorax sp CF313 | — | |
| Vibrio fluvialis 44473 Branch | — | |
| Xanthomonas campestris 37936 Branch | — | |
| Xanthomonas campestris pv raphani 756C | — | |

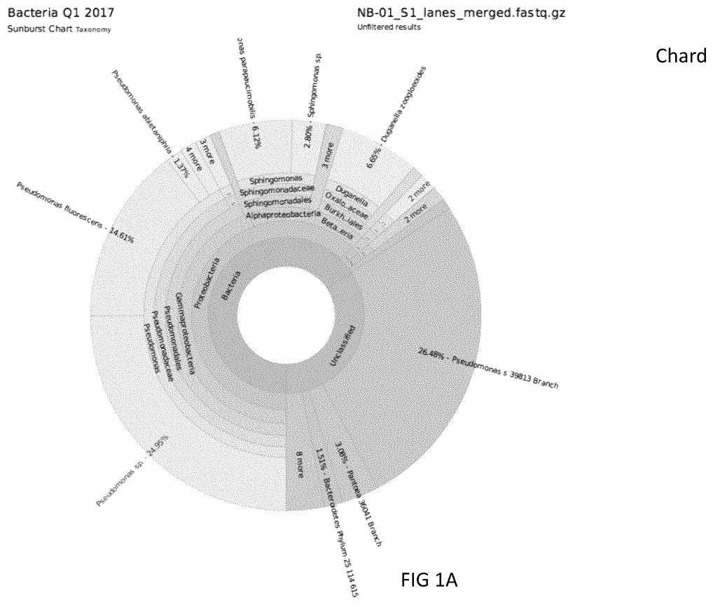

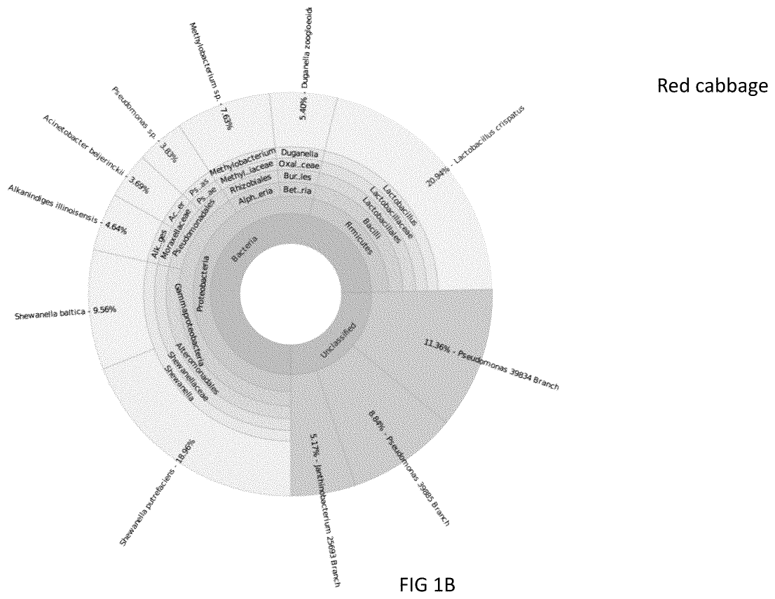

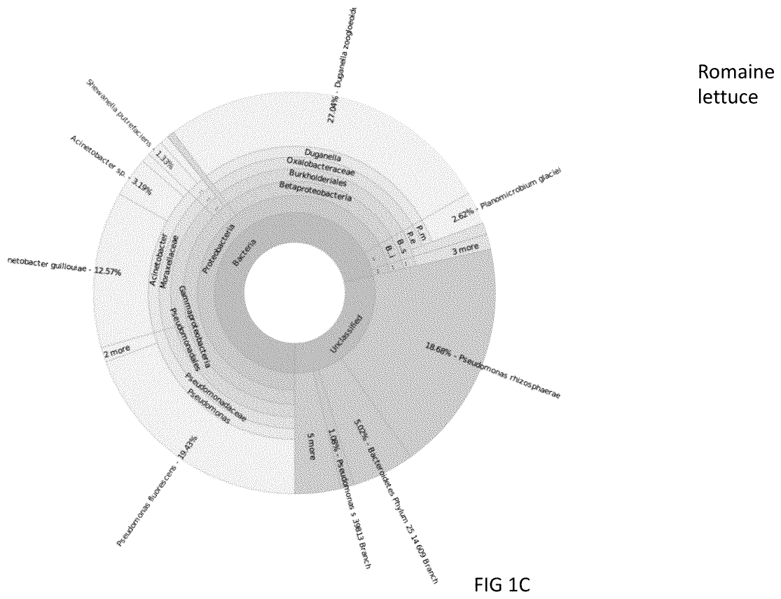

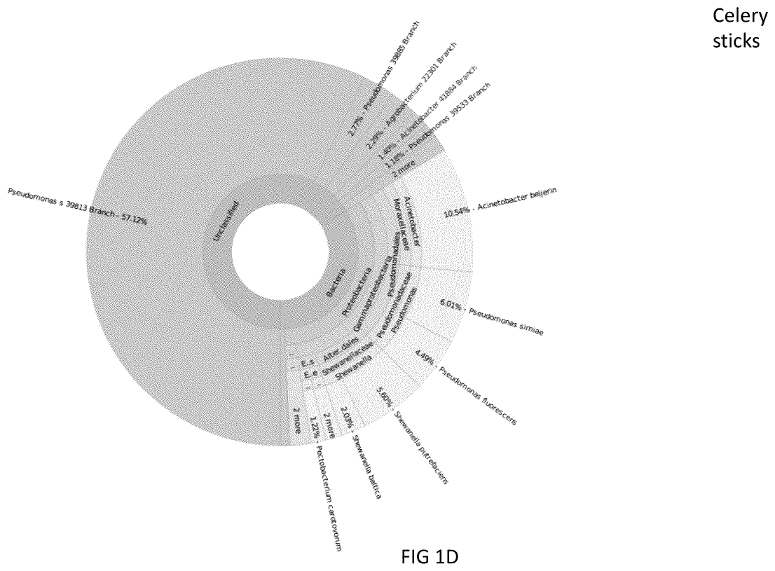

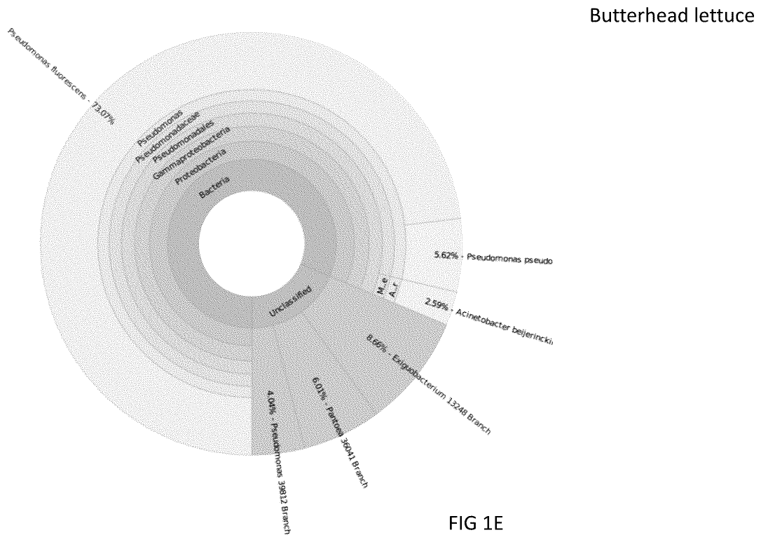

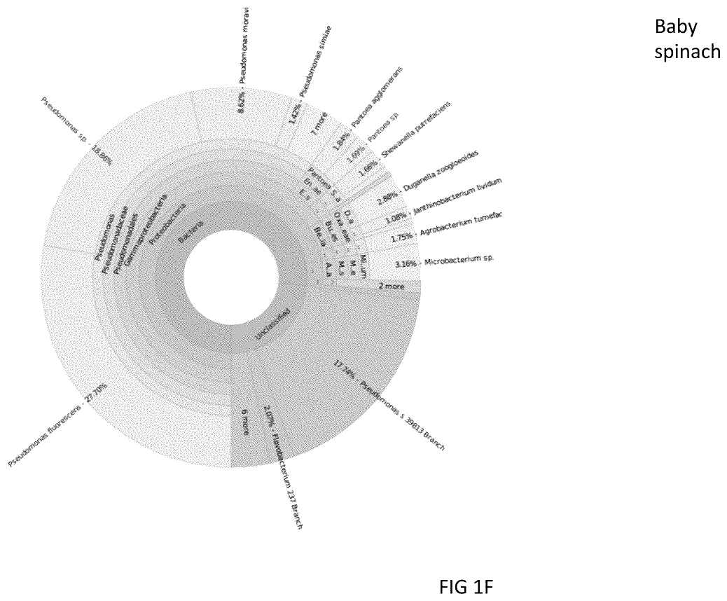

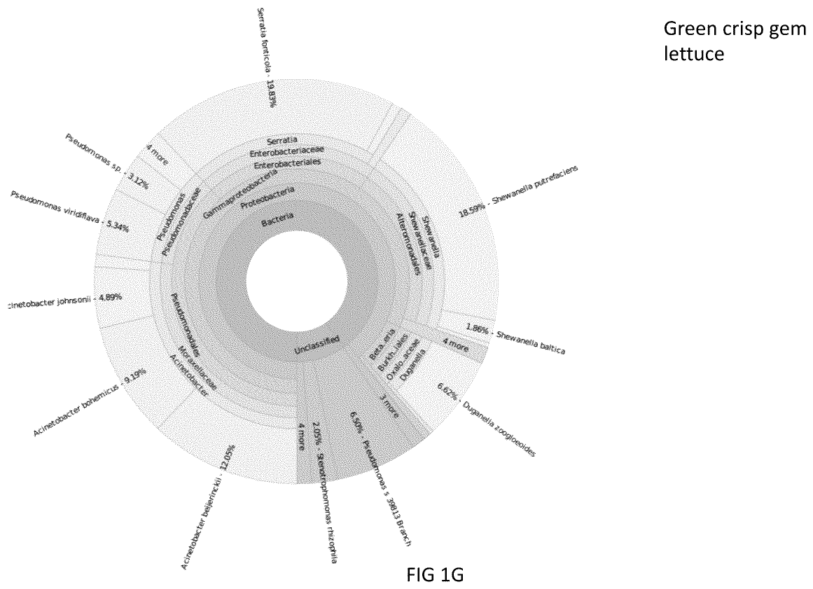

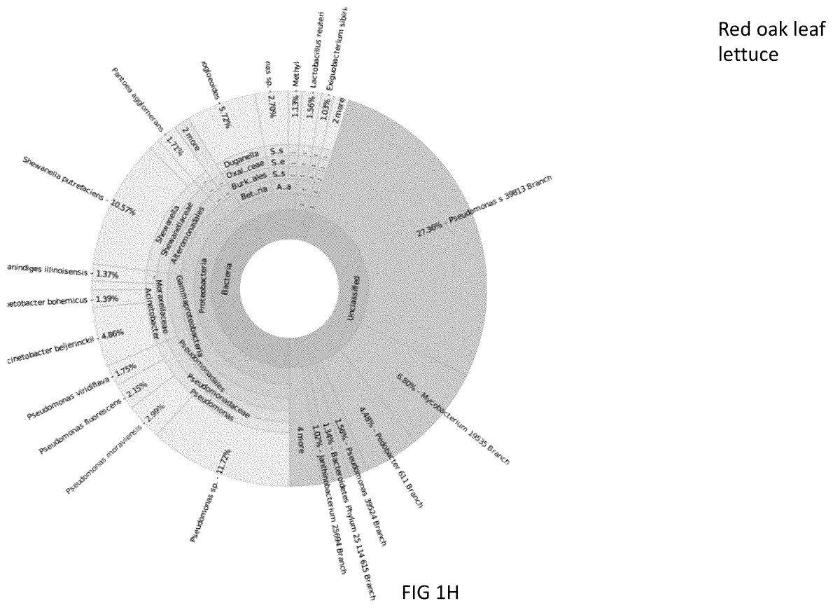

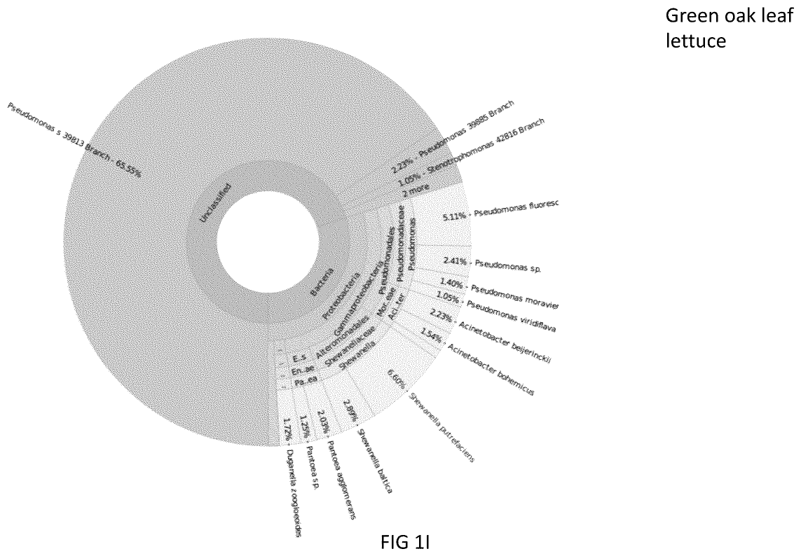

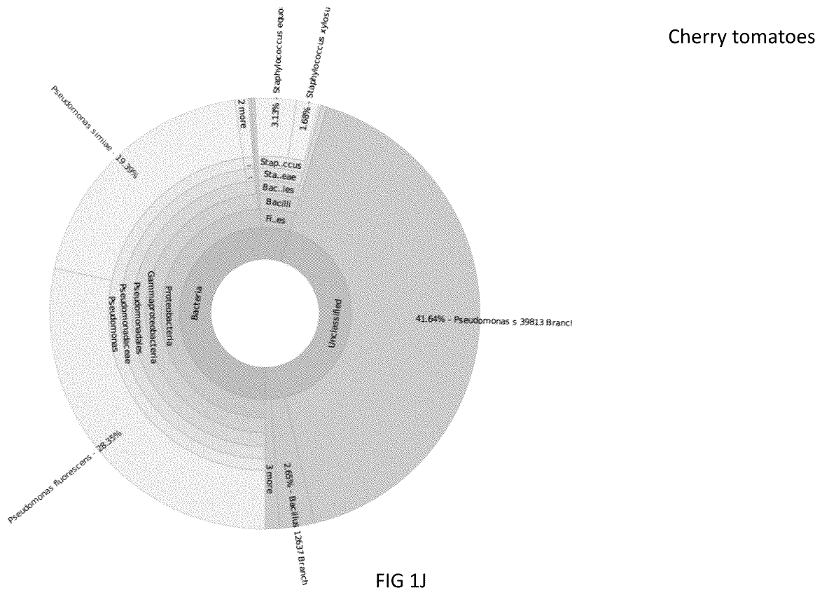

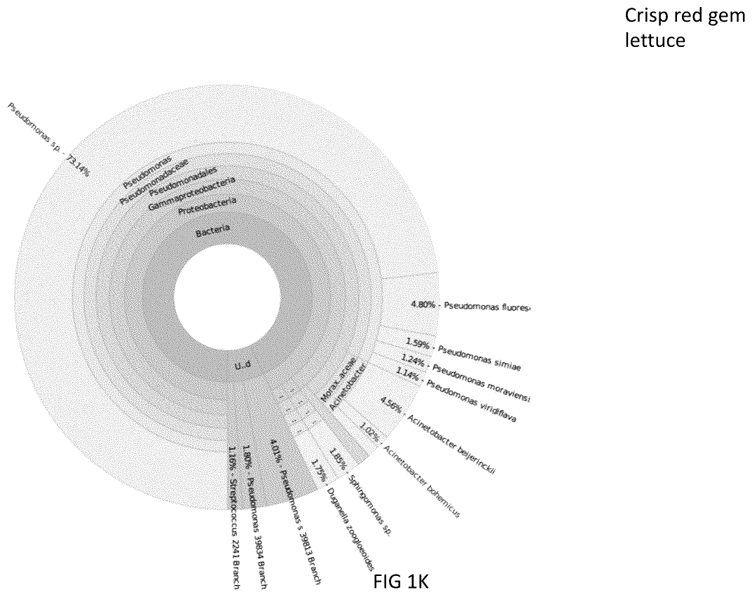

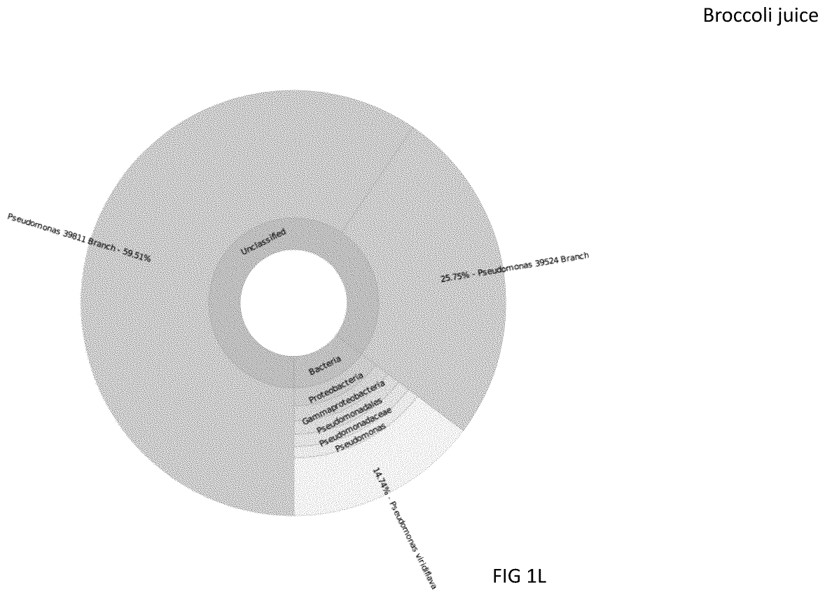

FIG. 1 shows bacterial diversity observed in a set of 21 plant-derived samples as seen by a community reconstruction based on mapping the reads from a shotgun sequencing library into the full genomes of a database containing 36,000 genomes by the k-mer method (CosmosID, OneCodex or Kraken2). The display corresponds to a sunburst plot constructed with the relative abundance for each corresponding genome identified and their taxonomic classification or pie charts. The genomes identified as unclassified have not been curated in the database with taxonomic identifiers and therefore not assigned to a group. This does not represent novel taxa and it is an artifact of the database updating process.

More specifically, FIG. 1A shows bacterial diversity observed in a green chard. The dominant group is gamma proteobacteria with different Pseudomonas species. The members of the group “unclassified” are largely gamma proteobacteria not included in the hierarchical classification as an artifact of the database annotation.

FIG. 1B shows bacterial diversity in red cabbage. There is a large abundance of Lactobacillus in the sample followed by a variety of Pseudomonas and Shewanella.

FIG. 1C shows bacterial diversity in romaine lettuce. Pseudomonas and Duganella are the dominant groups. A member of the Bacteroidetes was also identified.

FIG. 1D shows bacterial diversity in celery sticks. This sample was dominated by a Pseudomonas species that was not annotated yet into the database and therefore appeared as “unclassified” same for Agrobacterium and Acinetobacter.

FIG. 1E shows bacterial diversity observed in butterhead lettuce grown hydroponically. The sample contains relatively low bacterial complexity dominated by P. fluorescens and other groups. Also, there is a 9% abundance of Exiguobacterium.

FIG. 1F shows bacterial diversity in organic baby spinach. The samples were triple-washed before distribution at the point of sale and therefore it is expected that must of the bacteria detected here are endophytes. Multiple Pseudomonas species observed in this sample including P. fluorescens and other shown as “unclassified.”

FIG. 1G shows bacterial diversity in green crisp gem lettuce. This variety of lettuce showed clear dominance of gamma proteobacteria and with Pseudomonas, Shewanella, Serratia as well as other groups such as Duganella.

FIG. 1H shows bacterial diversity in red oak leaf lettuce. There is a relative high diversity represented in this sample with members of Lactobacillus, Microbacterium, Bacteroidetes, Exiguobacterium and a variety of Pseudomonas.

FIG. 1I shows bacterial diversity in green oak leaf lettuce. It is dominated Pseudomonas species including fluorescens and mostly gamma proteobacteria.

FIG. 1J shows bacterial diversity in cherry tomatoes. It is dominated by three species of Pseudomonas comprising more than 85% of the total diversity on which P. fluorescens comprises 28% of bacterial diversity.

FIG. 1K shows bacterial diversity in crisp red gem lettuce. Dominance by Pseudomonas species covering 73% of the bacterial diversity, on which P. fluorescens comprises 5% of bacterial diversity.

FIG. 1L shows bacterial diversity in broccoli juice. The sample is absolutely dominated by three varieties of Pseudomonas.

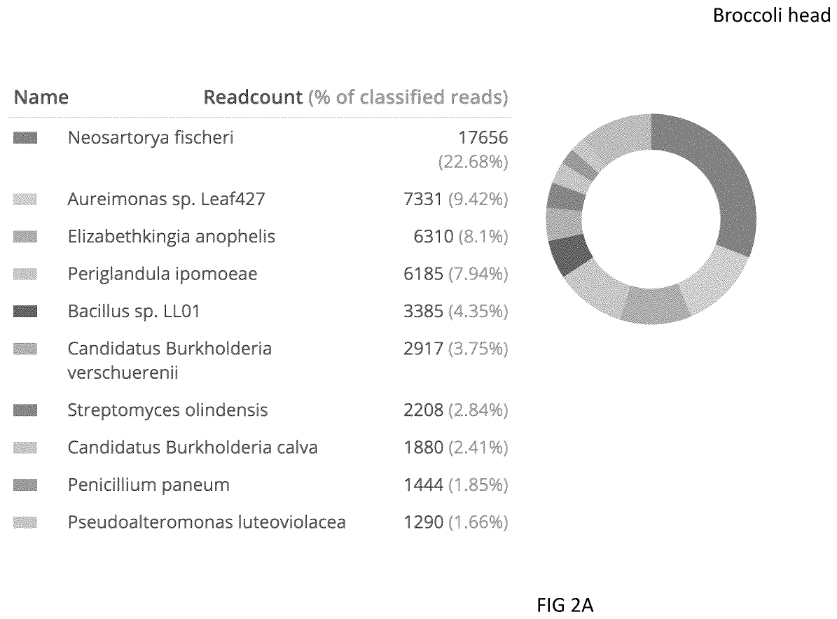

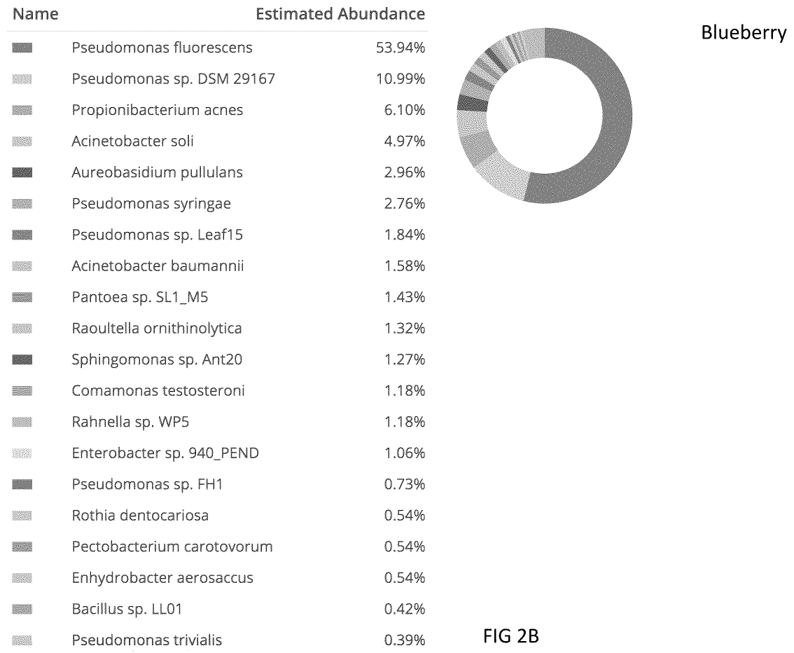

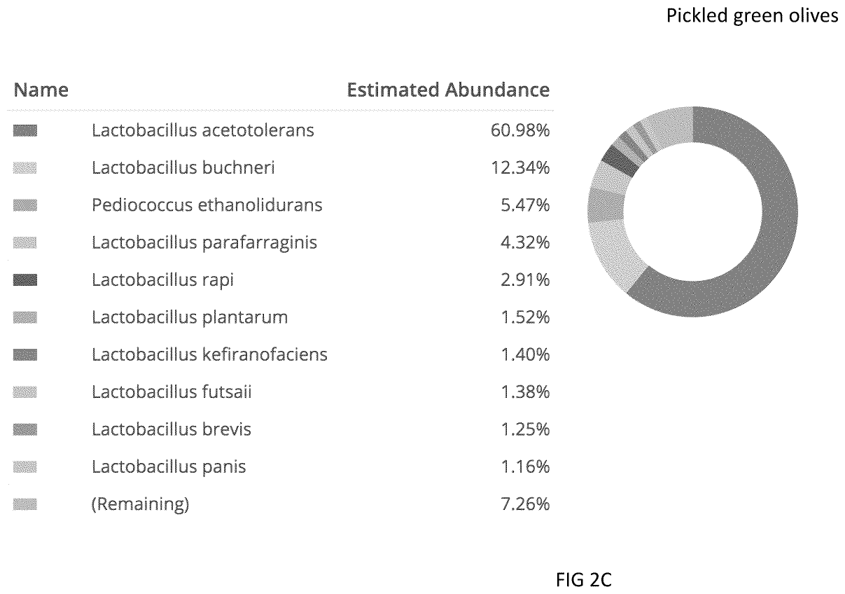

FIG. 2 shows taxonomic composition of blueberries, pickled olives and broccoli head. More specifically, FIG. 2A shows taxonomic composition of broccoli head showing a diversity of fungi and bacteria distinct from the broccoli juice dominated by few Pseudomonas species.

FIG. 2C shows taxonomic composition of blueberries.

FIG. 2C shows taxonomic composition of pickled olives showing a variety of lactic acid bacteria present and dominant. Some of the species are recognized as probiotics.

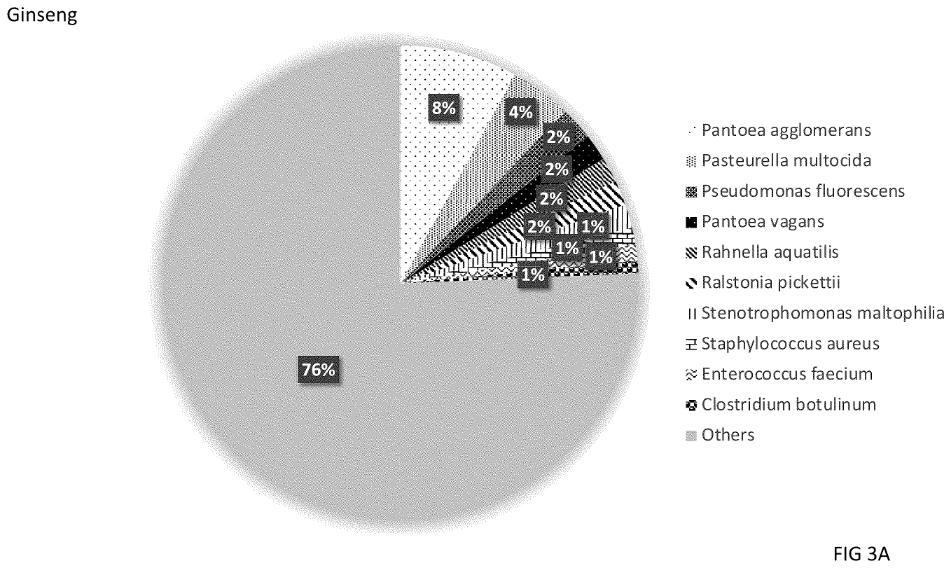

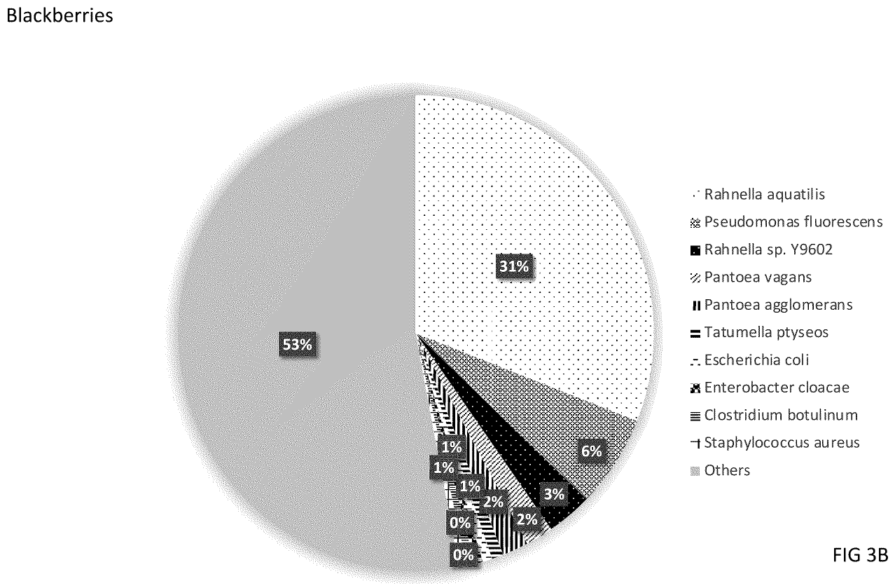

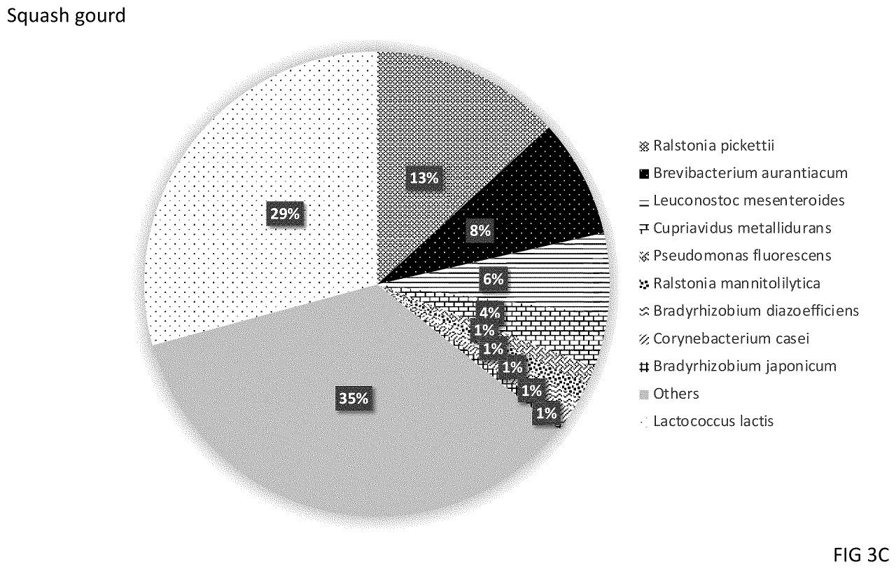

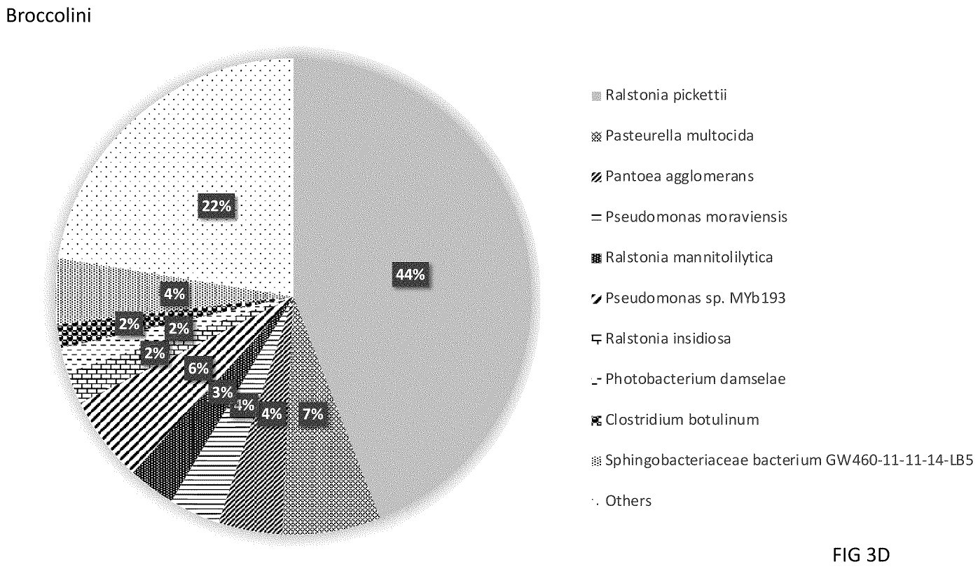

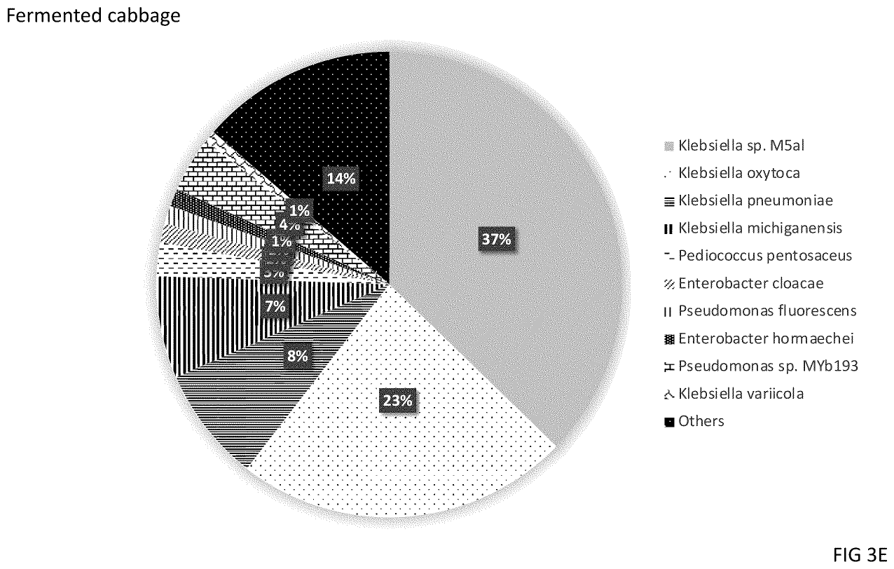

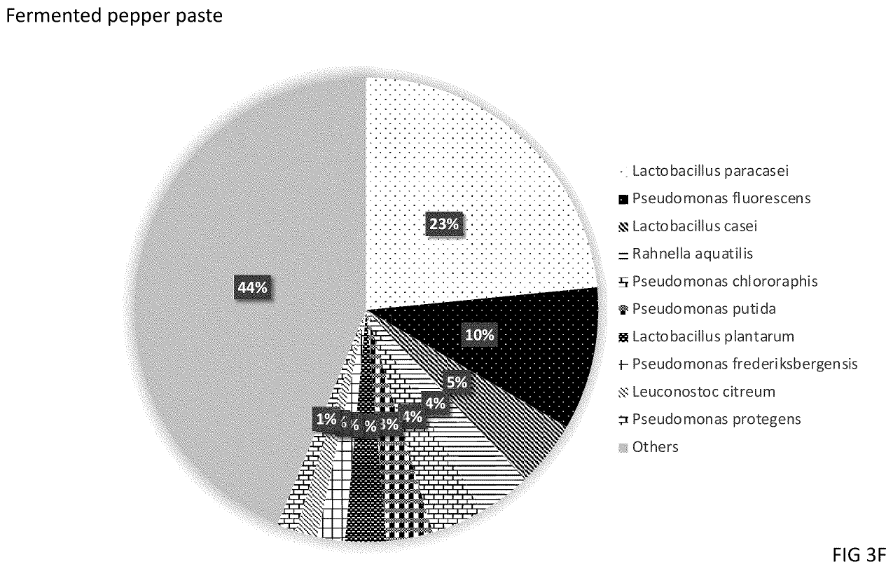

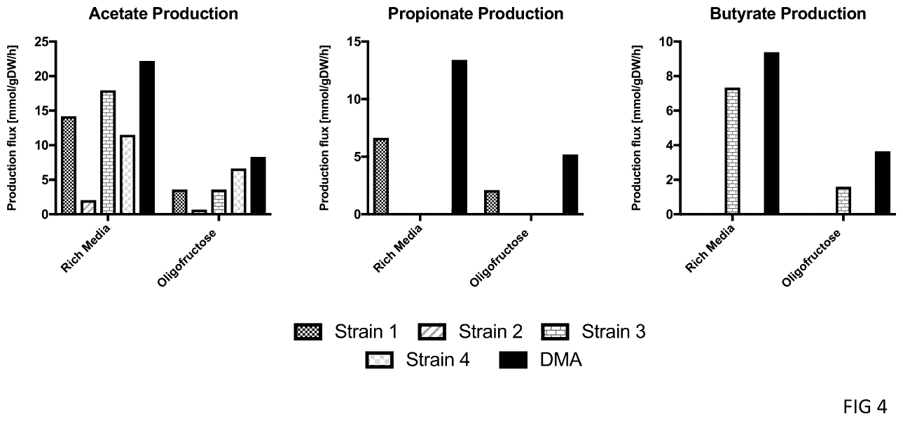

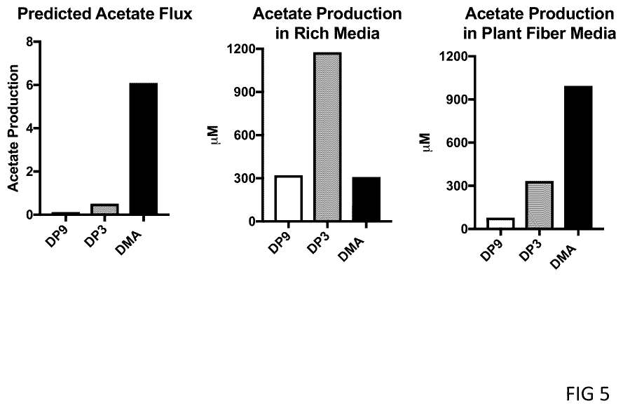

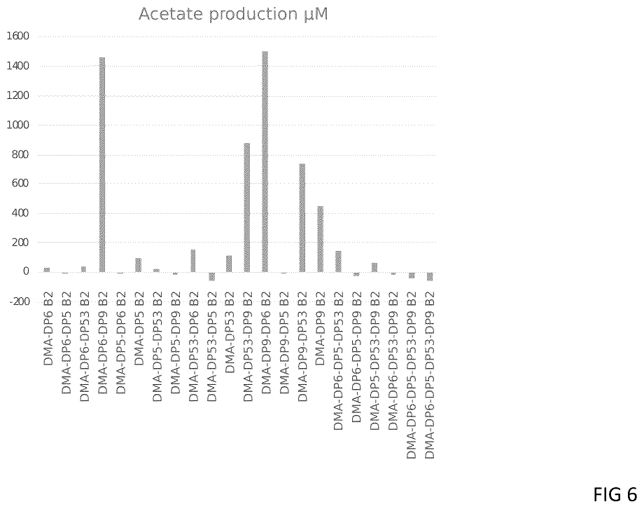

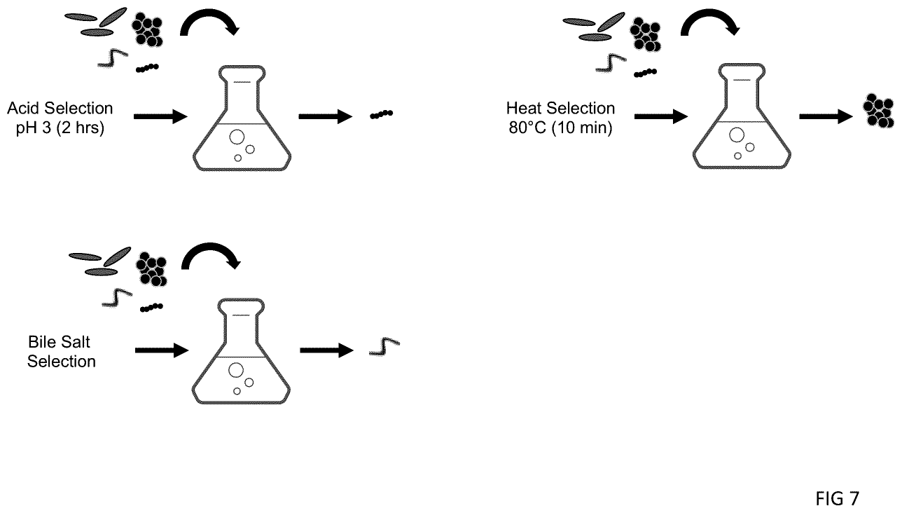

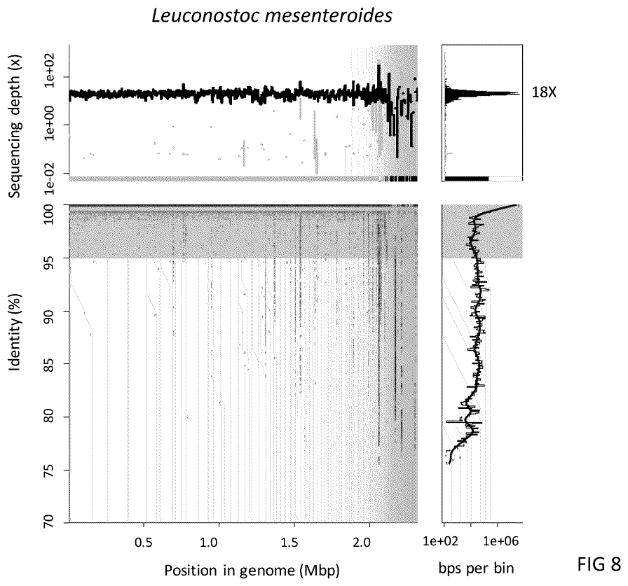

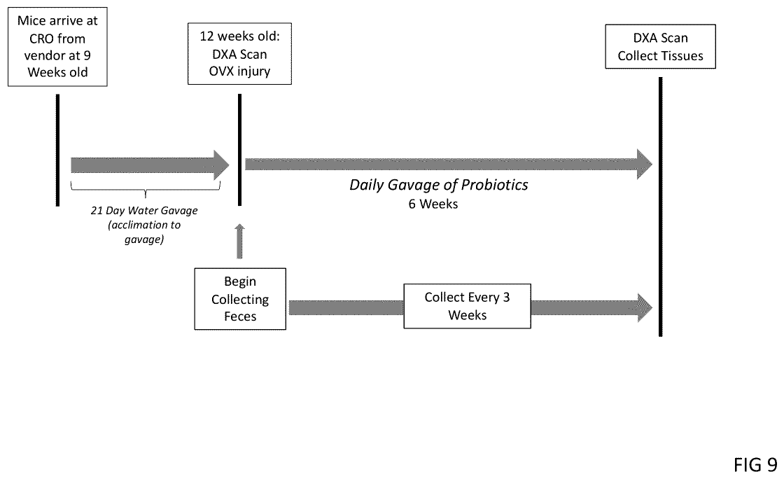

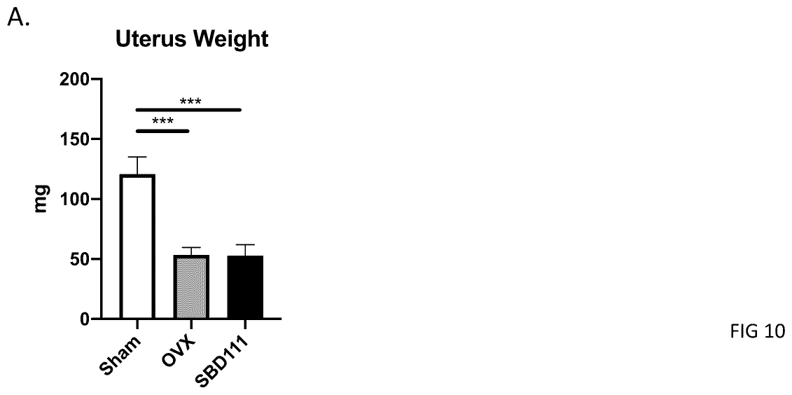

FIG. 3A shows taxonomic composition of ginseng. There is a relatively high diversity represented in this sample with members of Pseudomonas, Pantoea, and Stenotrophomonas.

FIG. 3B shows taxonomic composition of blackberries. The most abundant member is Rahnella aquatilis covering 31% of total composition.

FIG. 3C shows taxonomic composition of squash gourd. The sample is dominated by Lactococcus lactis covering 59% of total composition but also Leuconostoc mesenteroides was present at 3.3% of the bacterial population.

FIG. 3D shows taxonomic composition of broccolini. Ralstonia pickettii covers 44% of entire bacterial community.

FIG. 3E shows taxonomic composition of fermented cabbage. It contained Pediococcus pentosaceus as well as dominant gamma proteobacteria.

FIG. 3F shows taxonomic composition of fermented pepper paste. The sample enriched many lactic acid bacteria such as Lactobacillus paracasei, Lactobacillus casei and Lactobacillus plantarum.

### Example 2: In Silico Modeling Outputs for Different Assemblages and DMA Formulation

To generate in silico predictions for the effect of different microbial assemblages with a human host a genome-wide metabolic analysis was performed with formulated microbial communities selected from the Agora collection (Magbustoddir et al. 2016) and augmented with the genomes of bacterial members detected in the present survey. These simulations predict the “fermentative power” of each assemblage when simulated under different nutritional regimes including relatively high carbon availability (carbon replete) or carbon limited conditions when using plant fibers such as inulin, oligofructose and others as carbon source.

The method used for DNA sequencing the sample-associated microbiomes enabled to search for genes detected in the different vegetables related to propionate, butyrate, acetate and bile salt metabolism. This was done by mapping the reads obtained in the samples to reference genes selected for their intermediate role in the synthesis or degradation of these metabolites. There were organisms present in some of the 15 analyzed samples that matched the target pathways indicating their metabolic potential to produce desirable metabolites. Table 6a shows Metabolites in samples.

TABLE 6a
Metabolites in samples.

| NAME OF | ASSOCIATED | GENE | | E.C. | |
|---|---|---|---|---|---|
| ENZYME | METABOLITE | SYMBOL | PATHWAY | NUMBER | COMMENTS |
| ACETOLACTATE | (S)-2-ACETOLACTATE | | BUTANOATE | 2.2.1.6 | BUTYRATE |
| SYNTHASE I | | | METABOLISM | | PRODUCTION |
| ACETATE | PROPIONATE | ACKA | PROPANOATE | 2.7.2.1 | PROPIONATE |
| KINASE | | | METABOLISM | | |
| ACETYL-COA | PROPIONATE | AACS | PROPANOATE | 6.2.1.1 | PROPIONATE |
| SYNTHETASE | | | METABOLISM | | |
| ACETYL-COA | ACETATE | | PYRUVATE | 3.1.2.1 | ACETATE |
| HYDROLASE | | | METABOLISM | | |
| BILE SALT | BILE SALTS | ACR3 | BILE SALT | | BILE SALT |
| TRANSPORTER | | | TRANSPORT | | TOLERANCE |

### DMA Formulation

Microbes in nature interact with multiple other groups and form consortia that work in synergy exchanging metabolic products and substrates resulting in thermodynamically favorable reactions as compared to the individual metabolism. For example, in the human colon, the process for plant fiber depolymerization, digestion and fermentation into butyrate is achieved by multiple metabolic groups working in concert. This metabolic synergy is reproduced in the DMA concept where strains are selected to be combined based on their ability to synergize to produce an increased amount of SCFA when grown together and when exposed to substrates such as plant fibers.

To illustrate this process, a set of 40 bacterial and fungal strains were isolated from food sources and their genomes were sequenced. The assembled and annotated genomes were then used to formulate in silico assemblages considering the human host as one of the metabolic members. Assuming a diet composed of lipids, different carbohydrates and proteins the metabolic fluxes were predicted using an unconstrained model comparing the individual strain production of acetate, propionate and butyrate and compared to the metabolic fluxes with the assemblage.

In the first model, 4 strains were combined into a DMA. Strains 1˜4 are predicted to produce acetate as single cultures but the combination into a DMA predicts the flux will increase when modeled on replete media and the flux decreases when modeled on plant fibers. Strain 4 is predicted to utilize the fibers better than the other 3 to produce acetate. Strain 1 is the only member of the assemblage predicted to produce propionate and when modeled with the other 3 strains the predicted flux doubles in replete media and quadruples in the fiber media illustrating the potential metabolic synergy from the assemblage. Strain 3 is the only member of the assemblage predicted to produce butyrate and when modeled with the other 3 strains the predicted flux increase slightly in replete media and doubled in the fiber media illustrating the potential metabolic synergy from the assemblage.

TABLE 6b
Strains from first DMA model.

| | |
|---|---|
| Strain 1 | DP6 Bacillus cereus-like |
| Strain 2 | DP9 Pediococcus pentosaceus-like |
| Strain 3 | Clostridium butyricum DSM 10702 |
| Strain 4 | DP1 Pseudomonas fluorescens-like |

Substrate availability plays an important role in the establishment of synergistic interactions. Carbon limitation in presence of plant fibers favors fiber depolymerization and fermentation to produce SCFA. Conversely, carbon replete conditions will prevent the establishment of synergistic metabolism to degrade fibers as it is not favored thermodynamically when the energy available from simple sugars is available. To illustrate this, a DMA was formulated containing two strains of lactic acid bacteria and run a metabolic prediction assuming a limited media with plant fibers. According to the model, Leuconostoc predicted flux is higher than Pediococcus and the DMA flux increases five times on the combined strains. When tested in the lab and measured by gas chromatography, the acetate production increases 3 times compared to the single strains. However, when grown on carbon replete media with available simple sugars, acetate production is correspondingly higher compared to the plant fiber media but there is no benefit of synergistic acetate production when the two strains are grown together into a DMA.

In addition to acetate, propionate, and butyrate some strains produce other isomers. For example, strain DP1 related to P. fluorescens and DP5 related to Debaromyces hansenii (yeast) produce isobutyrate when grown in carbon-replete media as single strains, however there is metabolic synergy when tested together as DMA measured as an increase in the isobutyric acid production.

To describe experimentally the process of DMA validation the following method is applied to find other candidates applicable to other products:
1. Define a suitable habitat where microbes are with the desirable attributes are abundant based on ecological hypotheses. For example, fresh vegetables are known to have anti-inflammatory effects when consumed in a whole-food plant-based diet, and therefore, it is likely they harbor microbes that can colonize the human gut.
2. Apply a selection filter to isolate and characterize only those microbes capable of a relevant gut function. For example, tolerate acid shock, bile salts and low oxygen. In addition, strains need to be compatible with target therapeutic drugs including but not limited to bisphosphonates (alendronate, risedronate, ibandronate, zolendronate), biologics (denosumab, romosozumab), selective estrogen receptor mediators (Raloxifene), or anabolic agents (teriparatide, abaloparatide).
3. Selected strains are then cultivated in vitro and their genomes sequenced at 100× coverage to assemble, annotate and use in predictive genome-wide metabolic models.
4. Metabolic fluxes are generated with unconstrained models that consider multiple strains and the human host to determine the synergistic effects from multiple strains when it is assumed, they are co-cultured under simulated substrate conditions.
5. Predicted synergistic combinations are then tested in the laboratory for validation. Single strains are grown to produce a biomass and the spent growth media removed after reaching late log phase. The washed cells are then combined in Defined Microbial Assemblages with 2-10 different strains per DMA and incubated using a culture media with plant fibers as substrates to produce short chain fatty acids to promote gut health.
6. The DMAs are then analyzed by gas chromatography to quantify the short chain fatty acid production where the synergistic effect produces an increased production in the combined assemblage as compared to the individual contributions.

### Example 3: Gut Simulation Experiments

The experiment comprises an in vitro, system that mimics various sections of the gastrointestinal tract. Isolates of interest are incubated in the presence of conditions that mimic particular stresses in the gastro-intestinal tract (such as low pH or bile salts), or heat shock. After incubation, surviving populations are recovered. Utilizing this system, the impact of various oral anti-diabetic therapies alone or in combination with probiotic cocktails of interest on the microbial ecosystem can be tested. Representative isolates are shown in Table 7.

TABLE 7
Strains

| Strain | Heat | Isolation | Acid Shock | | |
|---|---|---|---|---|---|
| Number | Shock | Temperature | (pH 3; 2 hr) | Genus | Species |
| DP1 | No | 25 | No | Pseudomonas | fluorescens |
| DP2 | No | 37 | No | Hanseniaspora | occidentalis |
| DP3 | No | 25 | No | Leuconostoc | mesenteroides |
| DP4 | No | 25 | No | Aureobasidium | pullulans |
| DP5 | No | 37 | No | Debaromyces | hansenii |
| DP6 | Yes | 25 | No | Bacillus | wiedmannii |
| DP7 | No | 25 | No | Pichia | fermentans |
| DP8 | No | 25 | No | Hanseniaspora | opuntiae |
| DP9 | No | 25 | No | Pediococcus | pentosaceus |
| DP10 | Yes | 25 | No | Bacillus | velezensis |
| DP11 | No | 25 | No | Pseudomonas | putida |
| DP12 | No | 25 | Yes | Microbacterium | sp. |
| DP13 | No | 25 | Yes | Bacillus | mycoides |
| DP14 | No | 25 | Yes | Arthrobacter | luteolus |
| DP15 | No | 25 | No | Curtobacterium | sp. |
| DP16 | No | 25 | No | Lacihabitans | lacunae |
| DP17 | No | 25 | No | Rahnella | aquatilis |
| DP18 | No | 25 | No | Pseudomonas | sp. |
| DP19 | No | 25 | No | Curtobacterium | pusilium |
| DP20 | No | 25 | No | Stenotrophomonas | rhizophila |
| DP22 | No | 25 | No | Rahnella | sp. |
| DP23 | No | 25 | No | Erwinia | billingiae |
| DP24 | No | 25 | No | Filobasidium | globisporum |
| DP25 | No | 25 | No | Penicillium | solitum |
| DP26 | No | 25 | No | Methylobacterium | sp. |
| DP27 | No | 25 | No | Sphingomonas | sp. |
| DP28 | No | 25 | Yes | Aureobasidium | pullulans |
| DP29 | No | 25 | Yes | Pseudoclavibacter | helvolus |
| DP30 | No | 25 | Yes | Microbacterium | testaceum |
| DP31 | No | 25 | Yes | Sporisorium | reilianum |
| DP32 | No | 25 | No | Hafnia | paralvei |
| DP33 | No | 25 | No | Erwinia | persicinus |
| DP34 | No | 25 | Yes | Plantibacter | flavus |
| DP35 | No | 25 | Yes | Pantoea | ananatis |
| DP36 | No | 25 | Yes | Pantoea | vagans |
| DP37 | No | 25 | No | Pseudomonas | rhodesiae |
| DP38 | No | 25 | No | Rhodococcus | sp. |
| DP39 | No | 25 | No | Agrobacterium | tumefaciens |
| DP40 | No | 37 | No | Pantoea | sp. |
| DP41 | Yes | 37 | No | Corynebacterium | mucifaciens |
| DP42 | No | 37 | No | Pseudomonas | lundensis |
| DP43 | No | 25 | No | Janthinobacterium | sp. |
| DP44 | No | 25 | No | Herbaspirillum | sp. |
| DP45 | No | 25 | No | Sanguibacter | keddieii |
| DP46 | No | 25 | Yes | Pantoea | agglomerans |
| DP47 | No | 25 | Yes | Cronobacter | dublinensis |
| DP48 | Yes | 25 | No | Bacillus | paralicheniformis |
| DP49 | Yes | 25 | No | Bacillus | gibsonii |
| DP50 | No | 25 | No | Enterobacter | sp. |
| DP51 | No | 25 | No | Klebsiella | aerogenes |
| DP52 | No | 25 | No | Arthrobacter | sp. |
| DP53 | No | 25 | No | Pseudomonas | fragi |
| DP54 | No | 25 | No | Methylobacterium | adhaesivum |
| DP55 | Yes | 25 | No | Bacillus | megaterium |
| DP56 | Yes | 25 | No | Paenibacillus | lautus |
| DP57 | Yes | 25 | No | Bacillus | mycoides |
| DP58 | No | 25 | No | Janthinobacterium | svalbardensis |
| DP59 | No | 25 | No | Kosakonia | cowanii |
| DP60 | Yes | 25 | No | Bacillus | simplex |
| DP61 | No | 25 | No | Lelliottia | sp. |
| DP62 | No | 25 | No | Erwinia | sp. |
| DP63 | No | 25 | Yes | Pseudomonas | azotoformans |
| DP64 | No | 25 | No | Hanseniaspora | uvarum |
| DP65 | No | 25 | No | Bacillus | sp. |
| DP66 | No | 25 | No | Hanseniaspora | occidentalis |
| DP67 | Yes | 25 | No | Bacillus | sp. |
| DP68 | Yes | 25 | No | Bacillus | atrophaeus |
| DP69 | Yes | 25 | No | Bacillus | sp. |
| DP70 | No | 25 | No | Bacillus | subtilis |
| DP71 | No | 25 | No | Rhodotorula | sp. |
| DP72 | Yes | 25 | No | Bacillus | zhangzhouensis |
| DP73 | Yes | 37 | No | Bacillus | clausii |
| DP74 | Yes | 25 | No | Bacillus | coagulans |
| DP75 | No | 37 | No | Pseudomonas | gessardii |
| DP76 | No | 25 | No | Ochrobactrum | sp. |
| DP77 | Yes | 25 | No | Bacillus | aryabhattai |
| DP78 | No | 25 | No | Erwinia | rhapontici |
| DP79 | No | 25 | No | Pseudomonas | fragi |
| DP80 | No | 25 | No | Methylobacterium | adhaesivum |
| DP81 | Yes | 37 | No | Bacillus | clausii |
| DP82 | Yes | 37 | No | Bacillus | clausii |
| DP83 | Yes | 37 | No | Bacillus | clausii |
| DP84 | No | 25 | No | Microbacterium | sp. |
| DP85 | No | 30 | No | Methanolacinia | petrolearia |
| DP86 | No | 30 | No | Bacillus | velezensis |
| DP87 | No | 30 | No | Lactobacillus | plantarum |
| DP88 | No | 30 | No | Bacillus | velezensis |
| DP89 | No | 30 | No | Bacillus | subtilis |
| DP90 | No | 30 | No | Lactobacillus | plantarum |
| DP92 | No | 30 | No | Bacillus | subtilis |
| DP93 | No | 30 | No | Leuconostoc | mesenteroides |
| DP94 | No | 30 | No | Lactobacillus | brevis |
| DP95 | No | 30 | No | Lactobacillus | paracasei |
| DP96 | No | 30 | No | Lactobacillus | casei |
| DP97 | No | 30 | No | Lactococcus | garvieae |
| DP98 | No | 30 | No | Lactococcus | garvieae |
| DP100 | No | 30 | No | Lactobacillus | plantarum |
| DP101 | No | 30 | No | Pediococcus | pentosaceus |
| DP102 | No | 30 | No | Pichia | krudriazevii |

### Example 4: Computation of Microbial Average Nucleotide Identity (ANI)

A whole-genome based method was applied, known as the average nucleotide identity (ANI), to estimate the genetic relatedness among bacterial genomes and profile hundreds of microbial species at a higher resolution taxonomic level (i.e., species- and strain-level classification). ANI is based on the average of the nucleotide identity of all orthologous genes shared between a genome pair. Genomes of the same species present ANI values above 95% and of the same genus values above 80% (Jain et al. 2018).

Taxonomic annotation of the strains combined into DMAs using ANI and the NCBI RefSeq database indicated that these microbes represent species not present in the database and most likely are new bacterial species even when the nucleotide identity based on the 16S rRNA gene is 99%:

TABLE 8
Comparative predictive power of 16S rRNA sequence analysis and Average
Nucleotide Identity (ANI) analysis. While 16S rRNA sequence percentage
indicates a high degree of homology, ANI analysis demonstrates that
the overall genome sequence of the microbial entities isolated from
plants and described herein as compared to reference strains is different
enough in many cases to qualify as a different species.

| | | 16S rRNA | | ANI |
|---|---|---|---|---|
| ID | Name | gene (%) | Closest Ref. genome | (%) |
| DP3 | Leuconostoc | 99 | Leuconostoc | 91.77 |
| | mesenteroides | | pseudomesenteroides | |
| | (NR_074957.1) | | (JDVA01000001.1) | |
| DP9 | Pediococcus | 99 | Pediococcus pentosaceus | 99.6 |
| | pentosaceus | | (NC_022780.1) | |
| | (NR_042058.1) | 99 | Pseudomonas psychrophila | 86.82 |
| DP53 | Pseudomonas helleri | | (NZ_LT329795.1) | |
| | Pseudomonas | 99 | Pseudomonas antarctica | 94.48 |
| | (NR_148763.1) | | (NZ_CP015600.1) | |
| DP1 | fluorescens | 98 | Rahnella sp. | 88.24 |
| | (NR_115715.1) | | (NC_015061.1) | |
| DP22 | Rahnella sp. | | | |
| | (NR_025337.1) | | | |

### Example 5: Testing Composition Efficacy in a Mouse Model of Obesity Induced Bone Loss

Experimental Design: Male diet induced obese (DIO) and low-fat diet control C57BL/6J mice were purchased from the vendor at 16 weeks of age and were singly housed in individually ventilated cages (IVCs). At 5 weeks of age, mice were placed on either a low-fat diet (10% kcal, D12450B) or high-fat diet (60% kcal, D12492) (Open Source Diets; Research Diets Inc.) and remained on those respective diets for the duration of the experiment. Mice were allowed to acclimate for 2-weeks prior to the experimental initiation. At 18-weeks of age, one cohort of lean mice (N=4) and one cohort of obese mice (n=4) began control supplementation with water by daily oral gavage, while another group of obese mice (N=4) were treated with a daily oral gavage of SBD102 at a dose of 8×10¹⁰ CFUs/kg body weight. Control groups were provided sterile water at a dose of 5 mL/kg body weight. Mice were orally gavaged with control or test article daily for 8-weeks.

Bone mineral density analysis: At the time of sacrifice, mice were anesthetized via intraperitoneal injection of ketamine (60 mg/kg) and xylazine (4 mg/kg) and scanned by Dual Energy x-ray Absorptiometry (DEXA) scan (PIXImus2 Mouse Densitometer; GE) to measure whole body bone mineral density (BMD).

Distal femur trabecular bone analysis: To evaluate trabecular bone volumes at the distal femur, femurs were removed at the time of sacrifice and analyzed by micro computed tomography (microCT) with a Scanco microCT 40 desktop microCT scanner.

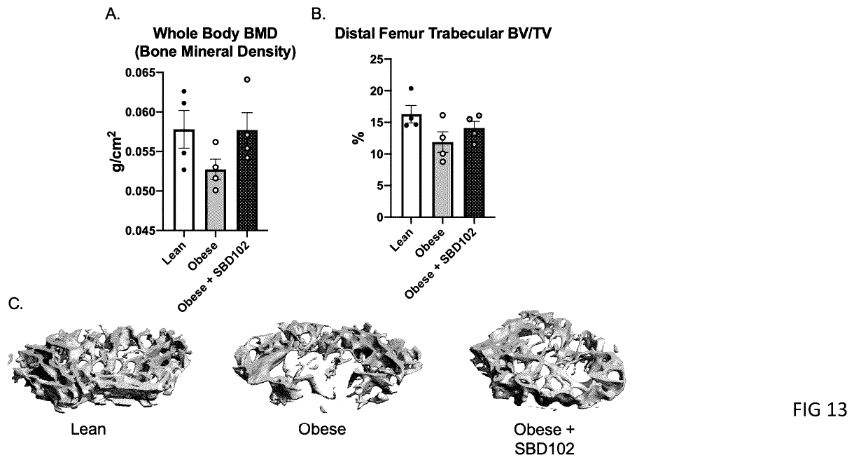

Conclusion: Compared to lean animals, obese mice lost 8.5% of their total BMD as measured by DXA scan. Obese mice treated with SBD102 were completely protected from this loss of BMD, indicating that SBD102 prevents obesity induced bone loss in a mouse model (FIG. 13A). Substantiating these data, microCT analysis of distal femurs showed similar trends where obesity induced a decrease in trabecular bone volume (BV/TV) compared to lean animals, and treatment with SBD102 prevented that decrease (FIG. 13B, 13C). With this, treatment with DMAs like SBD102 demonstrates a viable therapeutic option for the prevention of obesity induced bone loss.

SBD102 comprised DP9, DP2, and DP53.

### Example 6: Testing Composition Efficacy in Mouse Model of Postmenopausal Osteoporosis

Experimental Design: DMA compositions were evaluated for therapeutic efficacy in an ovariectomized (OVX) mouse model of postmenopausal osteoporosis. All mice were group-housed with 5 mice per cage in individually ventilated cages (IVCs) specifically designed for germ free husbandry [59, 60]. At 12-weeks of age, mice were weighed, had baseline feces collected, and underwent OVX surgery (N=20) or sham (N=10) surgery to deplete estrogen levels and commence the bone resorption process as previously described (Souza et al., 2019). 1-day post-surgery, mice were randomly divided into experimental groups and mice began a daily oral gavage regimen (200 uL) of saline (negative control, or SBD111 (5×10⁹ CFU/dose) which continued for 6-weeks. Fecal samples were collected at the beginning of the experiment, at week 3 and week 6 at the end of the experiment to monitor the composition of the gut microbiome over time. Finally, on the last day of the study, mice received a DXA scan to evaluate systemic BMD, followed by cuthanasia and collection of lumbar vertebra for analysis.

SBD111 comprised: DP1 (Pseudomonas sp.), DP94 (Lactobacillus brevis), DP95 (Leuconostoc mesenteroides), DP100 (Lactobacillus plantarum), and DP102 (Pichia krudriazevii).

Tissue collection and analysis: At the time of sacrifice, the uterus was removed and weighed to confirm that the ovaries were successfully removed, and estrogen was depleted following OVX surgery. Tissues were then collected from each mouse to evaluate bone quantity. Cecal contents were removed and flash frozen for downstream metagenomic sequencing and SCFA analysis by GC-FID to determine how our DMA impacted the composition and function of the gut microbiome. Finally, the lumbar spines were removed, processed, and analyzed analyzed by micro computed tomography (microCT) with a Scanco microCT 40 desktop microCT scanner.

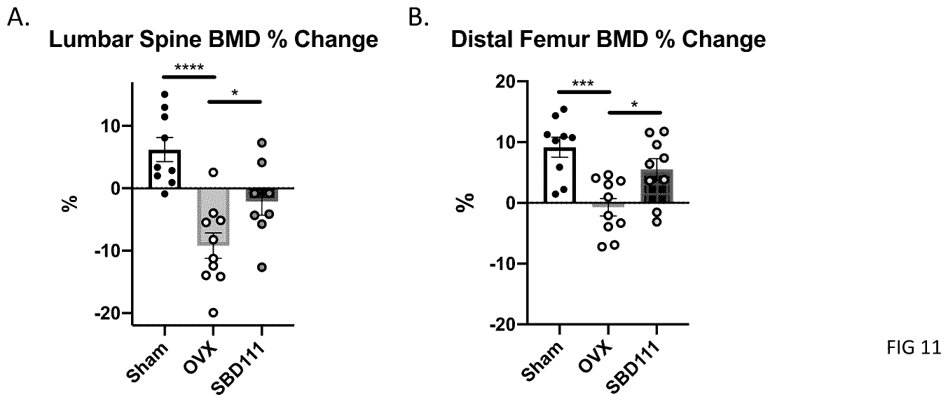

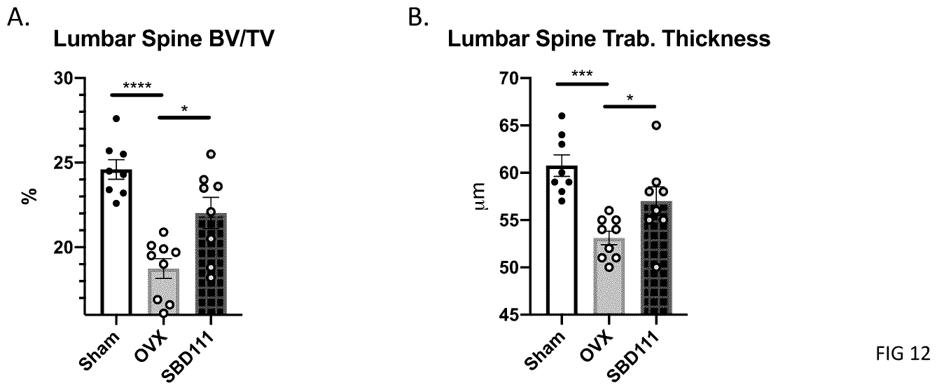

Conclusion: As has been previously described, OVX surgery induced a significant loss of BMD at the lumbar spine and distal femur in comparison to mice receiving sham surgery. Strikingly, OVX mice treated with SBD111 were almost completely protected from this steroid ablation induced bone loss after 6-weeks of daily treatment (FIG. 11A,B). Further, microCT analysis of the lumbar spine revealed significant protection from the loss of trabecular bone that is characteristic of this model in mice treated with SBD111. SBD111 treated animals retained ˜70% more trabecular bone volume than OVX controls, and also had thicker trabeculae compared to OVX animals (FIG. 12A, B). With this, DMA treatment demonstrates a potential viable therapeutic option for the protection against postmenopausal bone loss.

### Example 7: Orthopaedic Infection

To test the impact of our DMAs on the severity and incidence of implant-associated orthopedic infections, a well-recognized orthopedic implant surgery and infection model is used.

An orthopedic implant coated with Staphylococcus aureus (S. aureus) is generated by cutting flat stainless steel surgical wire into a 0.02×0.5×4 mm length, and bent at 1 mm to make an L shaped pin that is placed in an overnight culture of USA300 LAC::/uxmethicillin resistants S. aureus (2×10\hat{ }6 CFUs) for 20 minutes. Next, mice are anesthetized with an intraperitoneal injection of ketamine (60 mg/kg) and xylazine (4 mg/kg), and a 4 mm incision is made on the medial aspect of the right tibia. A hole in the medial tibia is then predrilled using successive 30- and 26-gauge needles before the infected pin is placed through the defect. The surgical site is then closed using a 5-0 nylon suture.

After surgery, mice are divided into treatment groups, and daily oral gavages of defined microbial assemblages (DMAs) or saline controls are performed for four weeks. Weekly fecal samples are collected for sequencing to monitor the gut microbiome over time. For each treatment, infections are monitored longitudinally by bioluminescence of the tibia using a Xenogen IVIS® camera system. At 14, 21, and 35 days post-infection, mice are euthanized and tissues are collected including ti bias for analysis of the infection by micro-computed tomography (MicroCT) and histology, serum for cytokine analysis, and colonic tissues for immune cell and cytokine evaluation by histology and qRT-PCR.

The results demonstrate that mice treated with the compositions disclosed herein have a shorter recovery period and milder infection symptoms than mice receiving the saline control.

### Example 8: Efficacy of DMAs on Improving Fracture Healing in a Mouse Model

To test the impact of DMAs on fracture healing, a well-recognized mouse model of fracture repair is used, the murine stabilized tibia fracture model. Here, 12-week old male and female mice are anesthetized with an intraperitoneal injection of ketamine (60 mg/kg) and xylazine (4 mg/kg). A 4 mm longitudinal incision is made on the anterior side of the right tibia, and a small hole is then be drilled into the tibial tuberosity using a 26-gauge needle. A transverse osteotomy is then performed with a number 11 scalpel blade at the proximal diaphysis of the tibia. The fibula remains intact. The bone fracture is then fixed with an intramedullary nailing procedure using a 26-gauge Quincke type spinal needle (BD Medical Systems), and the wound is closed using 5-0 nylon sutures. After surgery, mice are divided into treatment groups, and begin daily oral gavages for four weeks of defined microbial assemblages (DMAs) or saline controls. Weekly fecal samples are collected for sequencing to monitor the gut microbiome over time. For each treatment, Fractures are evaluated for strength, fracture callus formation, and union proficiency by X-ray, MicroCT, biomechanical torsion testing of the tibia, and histological/histomorphometric analysis of the tibia at 7, 14, 21, and 35 days post fracture. Additionally, serum, colon, and cecal material are collected from each mouse, from which serum is analyzed for inflammatory cytokine levels, colonic tissues are evaluated by qRT-PCR for immune cell and cytokine levels, and cecal material is shotgun sequenced for microbiome analysis.

The results demonstrate that mice treated with the compositions disclosed herein demonstrate the efficacy of DMAs on improving fracture healing in a mouse model.

### Example 9: Evaluation of Anti-Osteoarthritis Efficacy in a Mouse Model

DMAs are evaluated for their therapeutic efficacy in a mouse model of post-traumatic osteoarthritis. All mice are group housed with 3 mice per cage in individually ventilated cages (IVCs) specifically designed for germ free husbandry. At 12-weeks of age, mice have baseline feces collected, and receive either a sham injury (n=12) or a destabilization of the medial meniscus (DMM) (n=72) injury to induce arthritis. Briefly, a 5-mm-long incision is made through the skin on the medial side of the knee. Under a dissecting microscope, another incision through the synovial membrane is made along the medial side of the patellar tendon, opening the joint space. Using a #11 scalpel, the medial meniscotibial ligament (MMTL) is transected, enabling the medial meniscus to move freely. After surgery, 4-0 silk sutures are used to close the incision using an interrupted pattern. 1-week post-surgery, mice are randomly divided into experimental groups and fresh fecal samples are again collected. Mice then begin a daily oral gavage regimen (200 uL) of saline (negative control), DMA #1, DMA #2, DMA #3, DMA #4, or DMA #5 and continue for 12-weeks. Monthly fecal samples are collected to monitor the composition of the gut microbiome over time. On the last day of the study, mice are euthanized, and tissues are collected including serum, colon, cecal material, knees, and synovial membranes for analysis. Serum is analyzed for inflammatory cytokine levels; colonic tissues are evaluated by qRT-PCR for immune cell and cytokine levels; cecal material is shotgun sequenced for microbiome analysis; knees are evaluated by histology for total cartilage area and hypertrophic chondrocyte markers, and synovial membranes are assessed by qRT-PCR for inflammatory cytokine levels.

The results demonstrate that mice treated with the compositions disclosed herein anti-osteoarthritis efficacy in a mouse model.

### Example 10: Monitoring the Effect of DMAs on Microbial Flora of a Mammal

Alterations of the gut microbiota have been linked with changes in the host homeostasis such as chronic inflammation. In order to evaluate changes in the gut microbiota composition in OVX mice, fecal pellets were collected from OVX and sham mice during baseline and week 6 of treatment and the gut microbiota was characterized. Briefly, DNA was extracted using the ZymoBIOMICS DNA extraction Kit and quantified using a Qubit 2.0 flurometer with the dsDNA HS assay kit. Metagenomic libraries were prepared using the Illumina Nextera Flex DNA library preparation kit and an equimolar mixture of the libraries was sequenced on an Illumina NovaSeq S1 instrument on a 2×150 bp paired end run. Raw reads from the sequencing run were analyzed using SolexaQA (Cox et al. 2010) for trimming and removing of Illumina adaptors using a Phred score cutoff of 20 and minimum fragment length of 50 bp. Mouse sequencing reads were removed by mapping metagenomic reads against the Mus musculus genome GRCm38 using Bowtie2 with default parameters (Langmead et al. 2012). Taxonomic classification of the short-read metagenomes was determined using MetaPhlan2, which uses clade-specific marker genes from approximately 17,000 reference genomes to estimate the relative abundance of microbial members present in the sample (Troung et al. 2015).

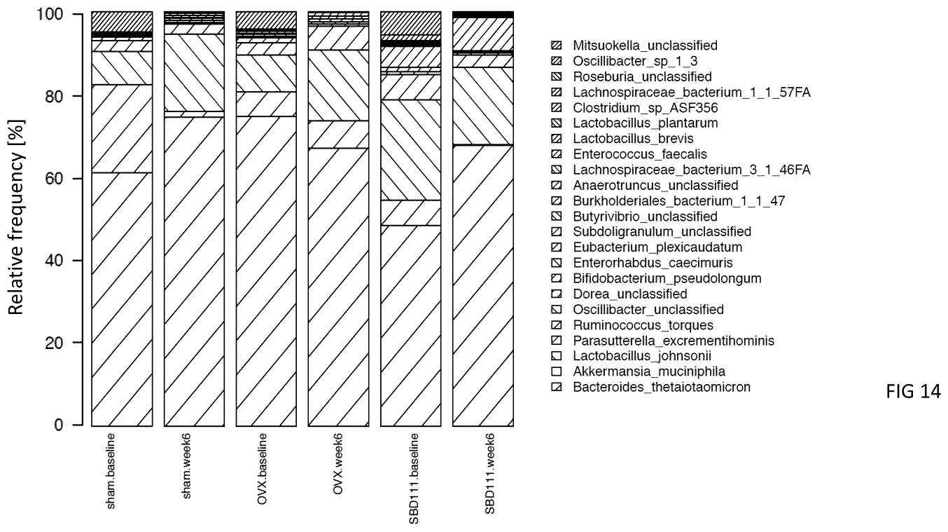

FIG. 14 shows the composition of the gut microbial community of the sham and ovx mice at the baseline and week 6 time points with different DMA combinations. Overall, Bacteroides thetaiotaomicron was the most prevalent taxon detected among the mice groups encompassing more than 50% of the total community on average, followed by Lactobacillus johnsonii with abundance values between 8.8% and 24.2%, excepting the sham baseline group where Akkermansia municiphila was the second most abundant taxon (21.3% on average). In the case of the SBD111 group, Bifidobacterium pseudolongum showed an increase in abundance at week 6 (from 5% to 7.8% of the total community). Bifidobacterium pseudolongum has been shown previously to modulate the immune system and decrease systemic inflammation. Inflammation plays a large role in osteoclastogenesis and the breakdown of bone, so the increased abundance of Bifidobacterium pseudolongum likely decreases systemic inflammatory mediators and thus decreases the resorption of bone, leading to improved BMD and trabecular bone volume in mice treated with SBD111 compared to OVX mice.

Further, Eubacterium plexicaudatum and Lactobacillus johnsonii increase in the sham group after 6 weeks while Akkermansia muciniphila decreases. These changes should be considered as part of a growth changes in the microbiome due to age and not associated to a changing phenotype due to the interventions.

In the OVX group there is a decrease in the Burkholderiales bacterium and Oscillibacter sp. SBD111 exhibits an increase in Bacteroides thetaiotaomicron in Lachnospiracea bacterium and a decrease in Burkholderiales, Parasutterella excremintihomini that can be associated to the phenotypes of prevention to bone loss.

### Example 11: Functional Profile of the Gut Microbiota Under DMA Treatment L-Rhamnose Degradation

In order to compare the rhamnose degradation in each group at week 6, relative abundances of genes related to L-rhamnose degradation pathway in individual mouse were calculated by mapping of sequencing reads against UniRef90 using HuMANN2 and characterizing gene families (Franzosa et al., 2018). Gene families that were annotated to the same MetaCyC reaction ID were averaged in each individual.

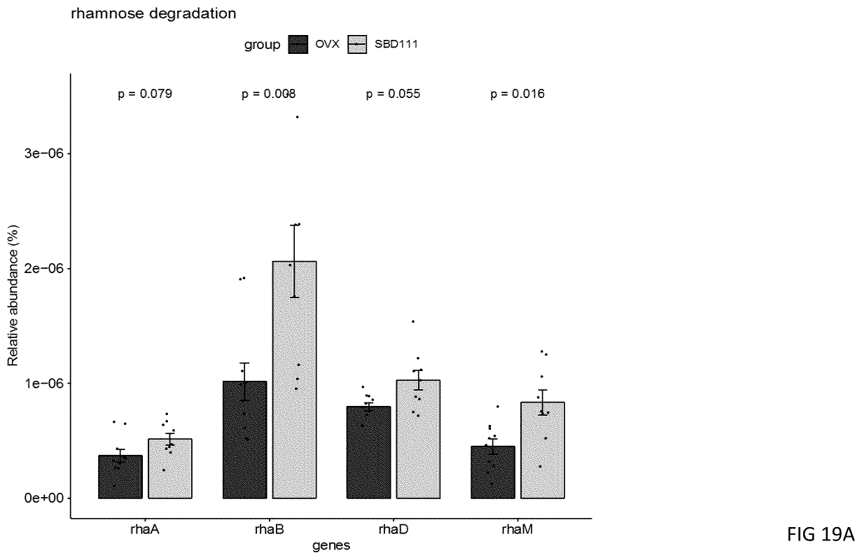

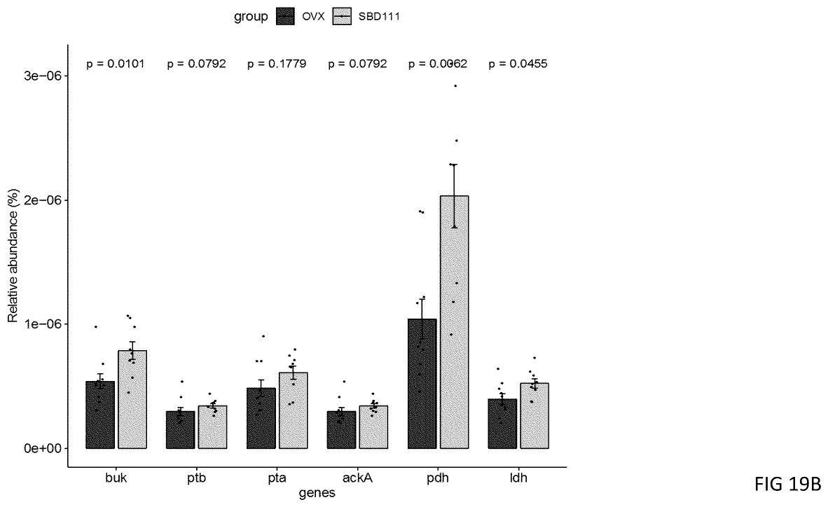

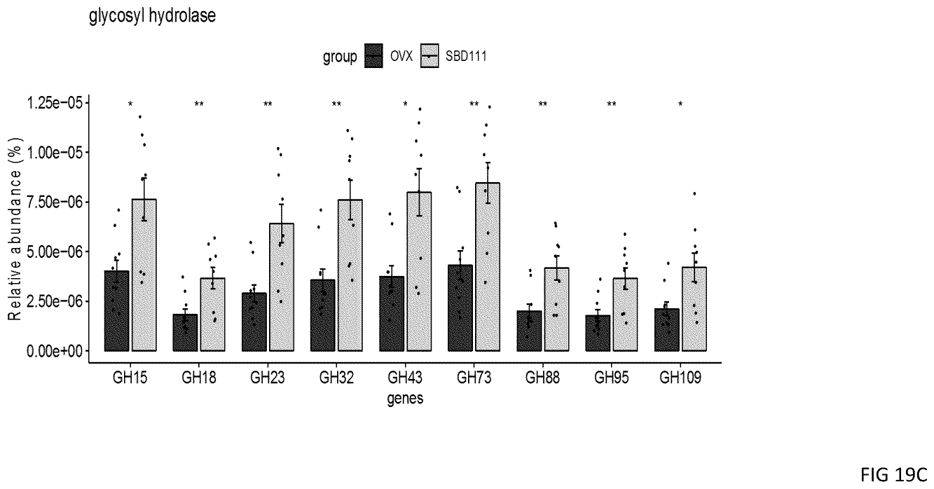

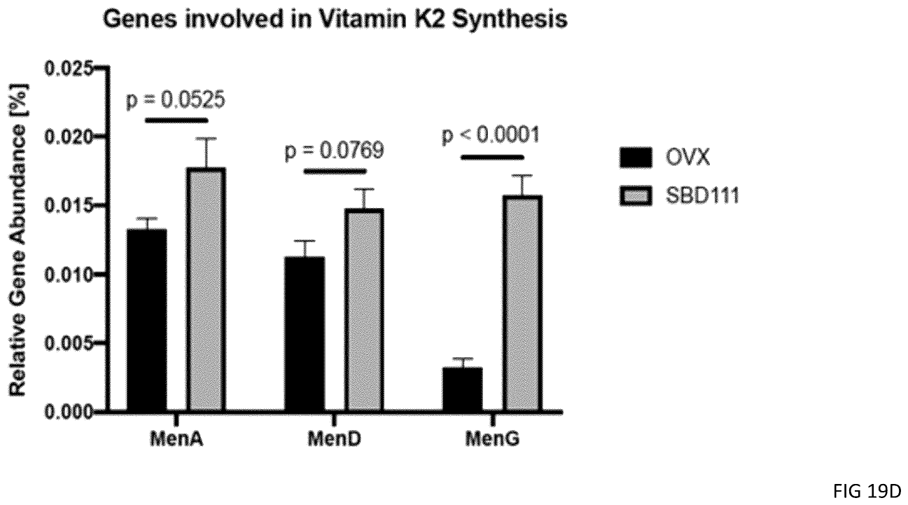

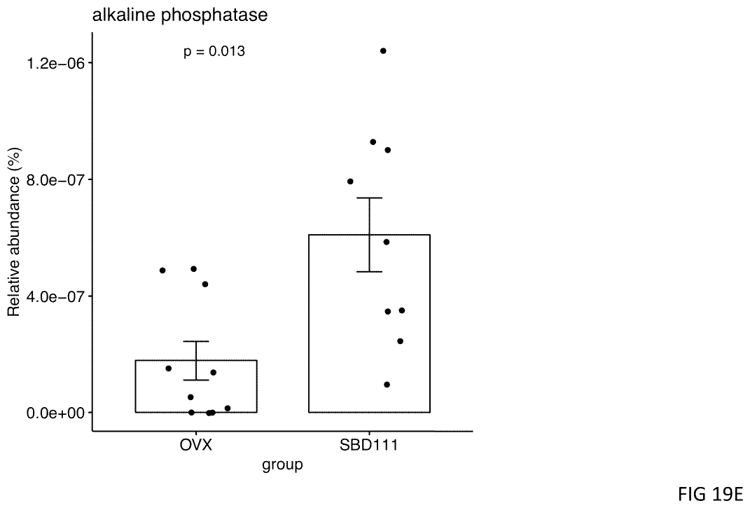

FIG. 19A shows the comparison of relative abundance of genes related to L-rhamnose degradation between OVX group and SBD111-treated group. Differences between groups were assessed by Mann-Whitney U test. Comparison of gene abundance between OVX and SBD111 groups indicated significantly higher abundance at week 6 in the SBD111 group in comparison with the OVX one. The L-rhamnose degradation pathway has been implicated in increased short chain fatty acid production. Further, increased short chain fatty acid production has been shown to improve BMD in OVX mice, and thus an increase in L-rhamnose degradation may partially explain the increased BMD and trabecular bone volume in SBD111 treated mice via increased SCFA production.

rhaD, rhamnulose-1-phosphage aldolase; rhaB, rhamnulokinase; rhaA L-rhamnose isomerase; rhaM, L-rhamnose mutarotase.

TABLE 9
Pathways significantly enriched or depleted after 6 weeks in both SBD111
and OVX in response to ovariectomy surgery and treatment. The mean relative
frequency between baseline and six- week time points were compared for each
mice group (OVX, sham, and SBD111). Mann-Whitney U test (P < 0.05).

| | OVX | sham | p-values |
|---|---|---|---|
| L-rhamnose degradation I | 0.57 | 0.77 | 0.02 |

| | OVX | DMA5 | |
|---|---|---|---|
| L-arginine biosynthesis I (via L-ornithine) | 0.80 | 0.58 | 0.01 |
| L-arginine biosynthesis II (acetyl cycle) | 0.41 | 0.26 | 0.02 |
| L-arginine biosynthesis III (via N-acetyl-L-citrulline) | 0.42 | 0.26 | 0.01 |
| L-rhamnose degradation I | 0.57 | 0.82 | 0.03 |
| dTDP-L-rhamnose biosynthesis I | 0.86 | 0.58 | 0.05 |

### Short-Chain Fatty Acids (SCFA) Gene Abundance

SCFA, produced mainly from microbial fermentation of dietary fiber, appear to be a major mediator of the beneficial effects induced by the gut microbiome (Tan et al., 2014). In order to compare the potential production level of short-chain fatty acids in each group at week 6, relative abundances of marker genes related to SCFA productions in individual mouse were calculated by mapping of sequencing reads against UniRef90 using HuMANN2 HUMAnN2 and characterizing gene families (Franzosa et al. 2018). Gene families that were annotated to the same MetaCyC reaction ID were averaged in each individual.

FIG. 19B shows the comparison of relative abundance of genes related to SCFA production between OVX group and SBD111-treated group. Differences between groups were assessed by Mann-Whitney U test. Butyrate kinase (buk) and phosphotransbutyrylase (ptb) were selected as marker genes representing butyrate production. Pyruvate dehydrogenase (pdh), phosphate acetyltransferase (pta) and acetate kinase (ackA) represent acetate production. L-lactate dehydrogenase (ldh) are involved in lactate production pathway. All genes presented in the figure are significantly more abundant in SBD111-treated groups compared to OVX group. Increased short chain fatty acid production has been shown to improve BMD in OVX mice, and thus an increase in genes related to SCFA biosynthesis may partially explain the increased BMD and trabecular bone volume in SBD111 treated mice.

### Glycoside Hydrolase

Microbial fermentation of complex non-digestible dietary carbohydrates and host-derived glycans in human intestines has important health consequences. Bacteria that colonize the mammalian gut possess large number of genes that encode carbohydrate active enzymes, which play an important role in the community by initiating the breakdown of complex substrates such as plant cell walls, starch particles and mucins.

Glycoside hydrolases (GH) are one of the carbohydrate active enzyme families that catalyze the hydrolysis of glycosidic bonds in plant fibers. In order to compare the potential capabilities of glycoside hydrolase activity in each group at week 6, relative abundances of gene families related to glycoside hydrolase in individual mouse were calculated by mapping sequencing reads against UniRef90 using HUMAN2 and characterizing gene families (Franzosa et al. 2018). UniRef90 gene families that were annotated to the same GH families were averaged in each individual.

FIG. 19C shows the comparison of relative abundance of genes related to glycoside hydrolase between OVX group and SBD111-treated group at week 6. Differences between groups were assessed by Mann-Whitney U test (*P<0.05, **P<0.01). Comparison of gene abundance between OVX and SBD111 groups indicated significantly higher abundance at week 6 in the SBD111 group in comparison with the OVX one. Results are shown in FIG. 19C. Increased abundance of GH genes likely indicates increased fermentation of non-digestible dietary fiber, leading to increased SCFA production. Increased short chain fatty acid production has been shown to improve BMD in OVX mice, and thus an increase in glycoside hydrolase may partially explain the increased BMD and trabecular bone volume in SBD111 treated mice through increased production of SCFA.

GH15, glucoamylase; GH18, chitinase; GH23, peptidoglycan lyase; GH32, invertase; GH43, β-xylosidase; GH73, lysozyme; GH88, unsaturated glucuronyl hydrolases; GH95, α-L-fucosidase; GH109, α-N-acetylgalactosaminidase.

### Vitamin K2 Biosynthesis

Functional characterization of the short-read metagenomes was determined using HUMAnN2 (Abubucker et al. 2012) with default parameters and the UniRef90 database (Suzek et al. 2015). Identifying genes involved in vitamin K2 (menaquinone) biosynthesis in the gut metagenomes is useful because vitamin K2 exhibits beneficial effects on human health. Although some studies have reported a positive effect of vitamin K2 consumption on bone health (Hess et al. 2015, Heaney 2013), the mechanism and factors involved in this relation are still unclear. Comparison of changes in gene abundance at the baseline and week 6 between OVX and SBD111 groups indicated higher significant increase in abundance at week 6 in the SBD111 group in comparison with the OVX one. Results are shown in FIG. 19D. As vitamin K2 has been shown to play a role in osteoblast functionality, an increase in Vitamin K2 biosynthesis may in part explain the increased BMD and trabecular bone volume in SBD111 treated mice compared to OVX mice.

### Alkaline Phosphatase

Alkaline phosphatase (ALP) is a ubiquitous membrane-bound glycoprotein that catalyzes the hydrolysis of phosphate monoesters at basic pH values and is produced by both eukaryotic and prokaryotic cells. In the intestine, ALP has been shown to improve intestinal barrier integrity, exerting its effects through dephosphorylation of proinflammatory molecules including lipopolysaccharide (LPS), flagellin, and adenosine triphosphate (ATP) released from cells during stressful events. Diminished activity of ALP could increase the risk of disease through changes in the microbiome, intestinal inflammation, and intestinal permeability. With this, the increased gene abundance of ALP

In order to compare the potential capabilities of alkaline phosphatase activity in each group at week 6, relative abundances of gene families related to alkaline phosphatase in individual mouse were calculated by mapping sequencing reads against UniRef90 using HUMAN2 and characterizing gene families (Franzosa et al., 2018).

FIG. 19E shows the comparison of relative abundance of alkaline phosphatase between OVX group and SBD111-treated group. Alkaline phosphatase was found to be increased in samples from SBD111 treated mice. Differences between groups were assessed by Mann-Whitney U test. With this, increased ALP in the microbiome could improve gut barrier integrity and decrease systemic inflammation, leading to improved BMD in SBD111 treated mice compared to OVX.

### Additional Changes

Changes in additional metabolic pathways were also observed. Some comparisons of interest are displayed in Table 10 and Table 11.

TABLE 10
Comparison of the pathways enriched or depleted in both SBD111
and OVX after 6 weeks with respect to baseline in response to
ovariectomy surgery and treatment. Mann-Whitney U test (P < 0.05).

| | | Baseline | 6 Weeks | |
|---|---|---|---|---|
| | | mean rel. | mean rel. | |
| | Pathway | freq. (%) | freq. (%) | p-values |
| DMA5 | L-arginine biosynthesis I (via L-ornithine) | 0.75 | 0.58 | 0.03 |
| DMA5 | L-arginine biosynthesis III (via N-acetyl-L-citrulline) | 0.36 | 0.26 | 0.04 |
| DMA5 | L-arginine biosynthesis IV (archaebacteria) | 0.82 | 0.64 | 0.03 |
| DMA5 | L-rhamnose degradation I | 0.48 | 0.82 | 0.01 |
| OVX | L-arginine biosynthesis I (via L-ornithine) | 0.58 | 0.80 | 0.02 |
| OVX | L-arginine biosynthesis II (acetyl cycle) | 0.21 | 0.41 | 0.00 |
| OVX | L-arginine biosynthesis III (via N-acetyl-L-citrulline) | 0.26 | 0.42 | 0.01 |
| OVX | L-arginine biosynthesis IV (archaebacteria) | 0.64 | 0.86 | 0.02 |

TABLE 11
Metabolic pathways of interest and observed changes in both SBD111
and OVX mice in response to ovariectomy surgery and treatment.

| Pathway | Metabolic effect | Observed changes |
|---|---|---|
| UMP biosynthesis | Immune system stimulation humans; DNA, | Increased in SBD111 at week 6 |
| | RNA synthesis | |
| coenzyme A biosynthesis II (mammalian) | Fatty acid metabolism cofactor | Increased in SBD111 at week 6 |
| adenine and adenosine salvage III | Nucleotide synthesis, immune system | Increased in SBD111 at week 6 |
| 5-aminoimidazole ribonucleotide biosynthesis II | Alternative glucose oxidation | Increased in SBD111 at week 6 |
| pentose phosphate pathway (non-oxidative branch) | Alternative glucose oxidation, active in | Increased in SBD111 at week 6 |
| | ovarian tissue, skeletal muscles | |
| L-rhamnose degradation I | Bone health, connective tissues, SCFA | Increased in SBD111 at week 6 |
| | upregulation | |
| superpathway of 5-aminoimidazole ribonucleotide | Purine biosynthesis | Increased in SBD111 at week 6 |
| biosynthesis | | |
| superpathway of L-aspartate and L-asparagine | Formation of succinate & fumarate in | Increased in SBD111 at week 6 |
| biosynthesis | anaerobic conditions | |
| L-arginine biosynthesis IV | BMD, immunomodulatory, anti-aging | Decreased in SBD111 at week 6 |
| L-arginine biosynthesis I (via L-orinthine) | Downstream intermediate releases acetate | Decreased in SBD111 at week 6 |
| flavin biosynthesis III (fungi) | Energetic metabolism, redox homeostasis | Increased in SBD111 at week 6 |
| | and protein folding, vitamin B2 | |
| L-histidinedegradation I | Catabolite repression, L-glutamate, amino | Increased in SBD111 at week 6 |
| | acid degredation | |
| D-fructuronate degredation | Female-specific factor, gut microbiota | Decreased in OVX vs SBD111 |
| inosine-5′-phosphate biosynthesis I | RNA and DNA synthesis, IMPDH in T cells, | Decrease in SBD111 vs OVX |
| | immune system | |
| sulfate reduction I (assimilatory) | Colonic sulfide metabolism, hydrogen | Appeared in OVX |
| | sulfide, intestinal disorders | |
| dTDP-L-rhamnose biosynthesis I | Enterobacterial common antigen | Increased in OVX vs SBD111 |
| tetrapyrrole biosynthesis I (from glutamate) | Production of vitamin B12, antioxidant | Decrease in SBD111 vs OVX |
| | properties | |
| L-arginine biosynthesis II (acetyl cycle) | Inflammation regulation | Increase in OVX |

TABLE 12
Comparison of genes enriched or depleted in SBD111 and OVX after 6 weeks with
respect to baseline in response to ovariectomy surgery and treatment. Mann-Whitney U test (P < 0.05).

| | | SBD111 | OVX | |
|---|---|---|---|---|
| | | mean rel. | mean rel. | |
| Family | Genes | freq. (%) | freq. (%) | p-values |
| UniRef90_D6D0Y9 | D6D0Y9_Alpha-1,2-mannosidase, putative | 0.017 | 0.014 | 0.037 |
| UniRef90_Q8A1H4 | Q8A1H4_Glycosyl hydrolase, family 88 | 0.018 | 0.015 | 0.039 |
| UniRef90_R9KRQ6 | R9KRQ6_Beta-galactosidase | 0.004 | 0.001 | 0.007 |
| UniRef90_Q8A1F2 | Q8A1F2_Phospholipid/glycerol acyltransferase | 0.017 | 0.014 | 0.035 |
| UniRef90_Q8A222 | Q8A222_N-actylgalactosamine-6-sulfatase | 0.017 | 0.013 | 0.033 |
| UniRef90_Q8A9I7 | Q8A9I7_dTDP-4-dehydrorhamnose | 0.014 | 0.010 | 0.017 |
| UniRef90_J9CIK2 | J9CIK2_Tetrapyrrole methylase family protein | 0.016 | 0.012 | 0.032 |
| UniRef90_R7KTS6 | R7KTS6_Zinc ABC transporter zinc-binding | 0.019 | 0.015 | 0.042 |
| UniRef90_R6UVU4 | R6UVU4_GDP-L-fucose synthase | 0.015 | 0.011 | 0.044 |
| UniRef90_Q8A3K1 | Q8A3K1_L-rhamnose-proton symporter | 0.017 | 0.013 | 0.043 |
| UniRef90_Q8A7Q2 | Q8A7Q2_Glycoside transferase family 2 | 0.017 | 0.012 | 0.027 |
| UniRef90_Q8A3K8 | Q8A3K8_Glycoside transferase family 4 | 0.019 | 0.015 | 0.021 |
| UniRef90_Q8A7J9 | Q8A7J9_Phosphatidylglycerophosphatase A | 0.016 | 0.011 | 0.028 |
| UniRef90_R5UG56 | R5UG56_Gliding motility-associated protein | 0.015 | 0.011 | 0.019 |
| UniRef90_Q8A677 | Q8A677_Guanylate kinase | 0.018 | 0.013 | 0.013 |
| UniRef90_Q8A3L8 | Q8A3L8_Glycoside transferase family 4 | 0.021 | 0.016 | 0.036 |
| UniRef90_Q5LI10 | Q5LI10_Argininosuccinate lyase | 0.017 | 0.013 | 0.043 |
| UniRef90_Q8AAL0 | Q8AAL0_Arabinose-proton symporter | 0.019 | 0.014 | 0.046 |
| UniRef90_Q8A0G3 | Q8A0G3_NADH-quinone oxidoreductase | 0.019 | 0.014 | 0.039 |
| UniRef90_Q8A0F5 | Q8A0F5_NADH-quinone oxidoreductase | 0.018 | 0.013 | 0.050 |
| UniRef90_Q8A3K7 | Q8A3K7_Glycoside transferase family 2 | 0.018 | 0.013 | 0.006 |
| UniRef90_D6D807 | D6D807_Asparaginase | 0.019 | 0.014 | 0.031 |
| UniRef90_R9H5P6 | R9H5P6_Serine acetyltransferase | 0.017 | 0.011 | 0.010 |
| UniRef90_R5B6J1 | R5B6J1_Tyrosine-tRNA ligase | 0.072 | 0.108 | 0.001 |
| UniRef90_R7J544 | R7J544_L-aspartate oxidase | 0.038 | 0.051 | 0.009 |

### Example 12: Increase in Relative Abundance and Intra-Population Diversity of Bifidobacterium pseudolongum in Week-6 SBD111-Treated Group

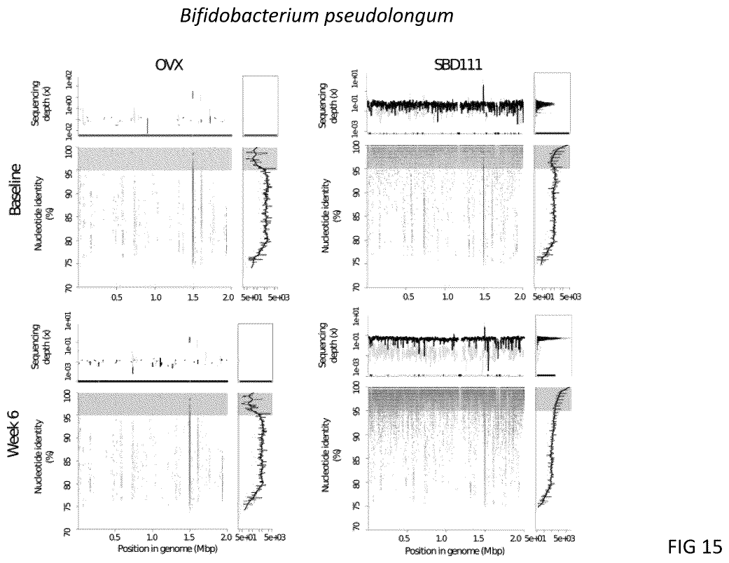

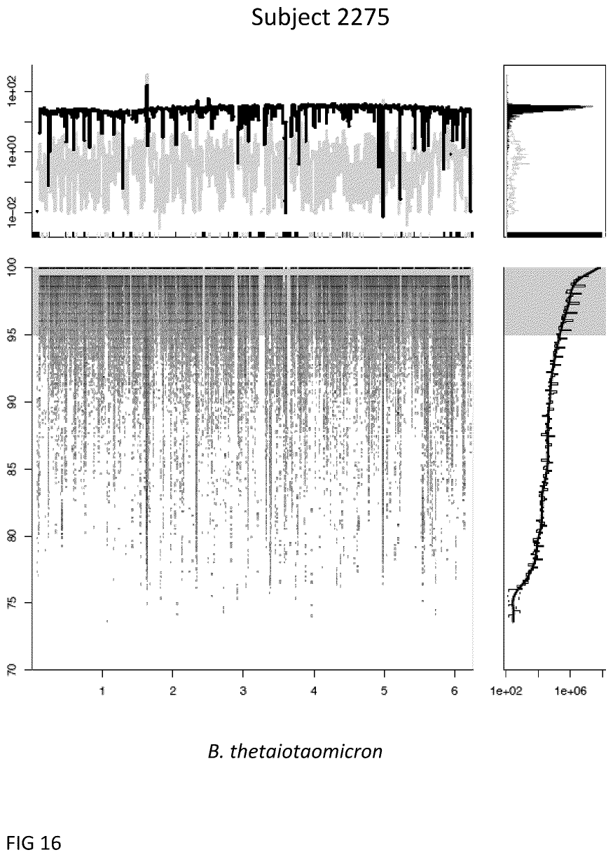

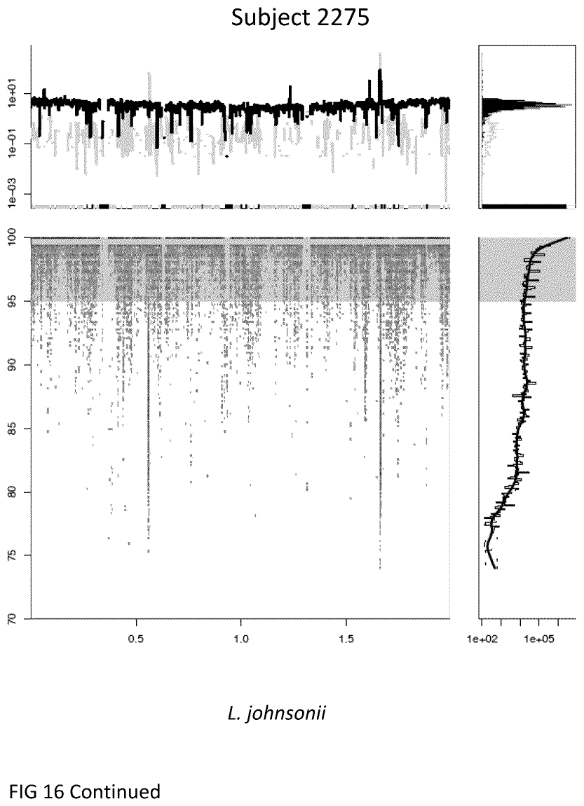

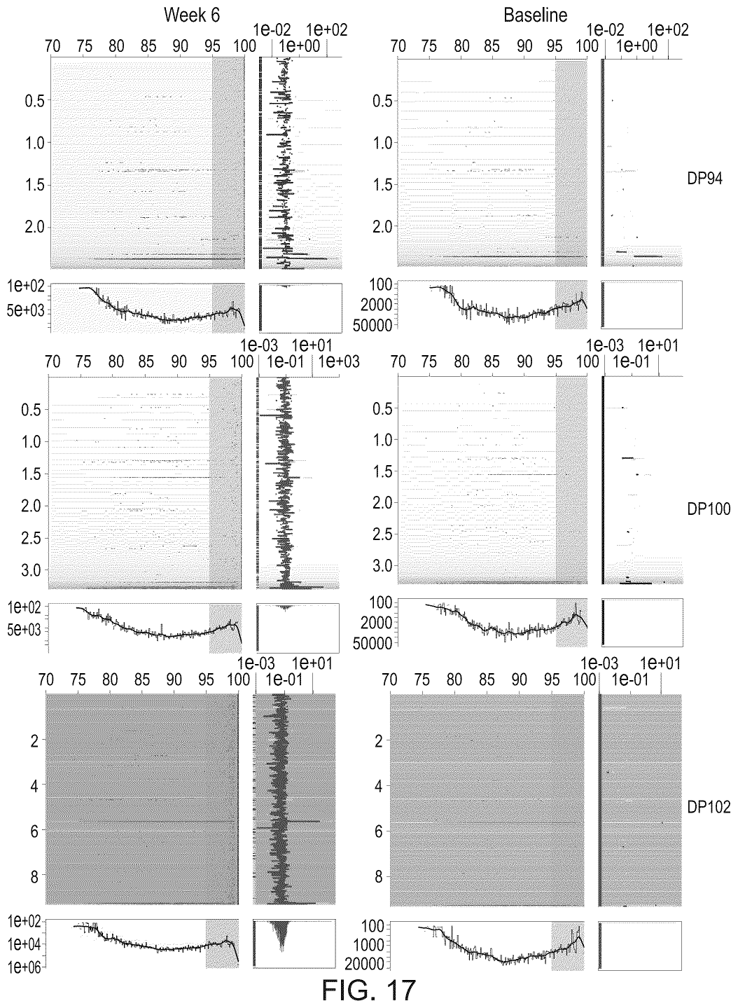

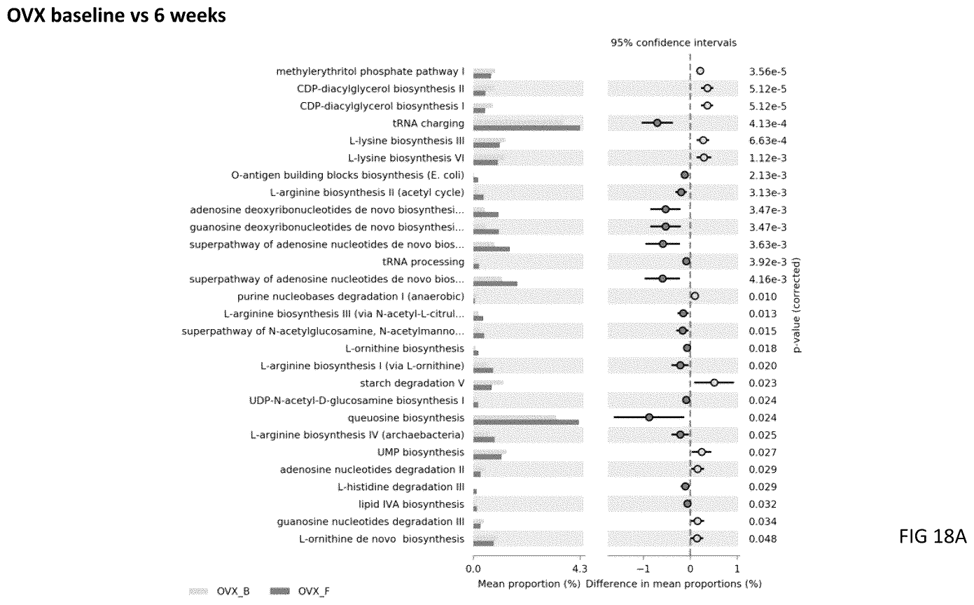

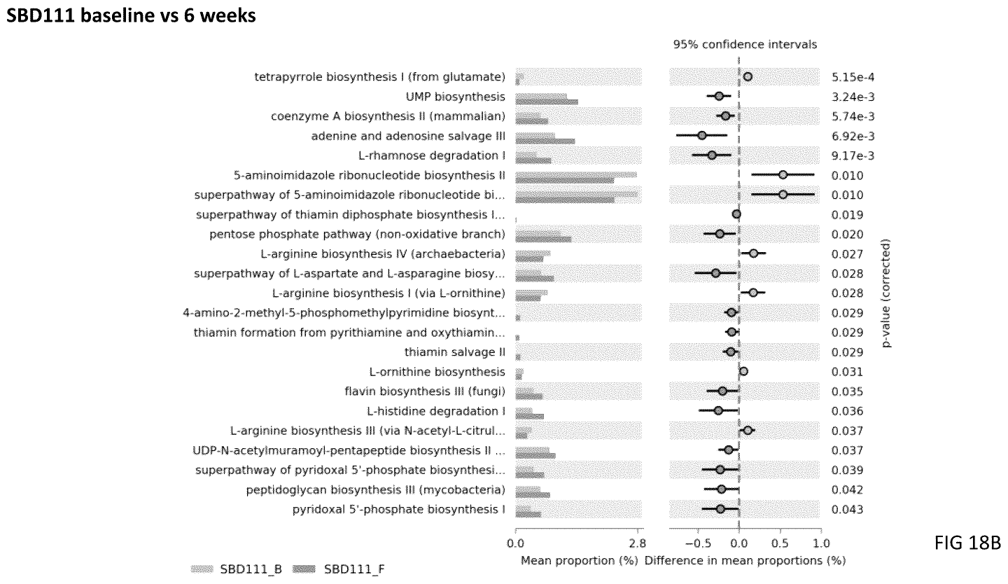

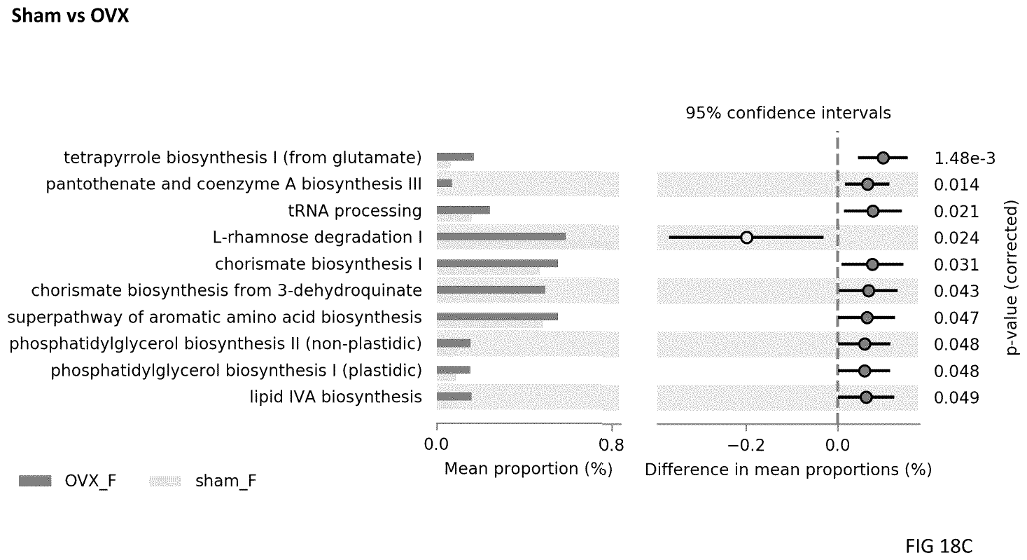

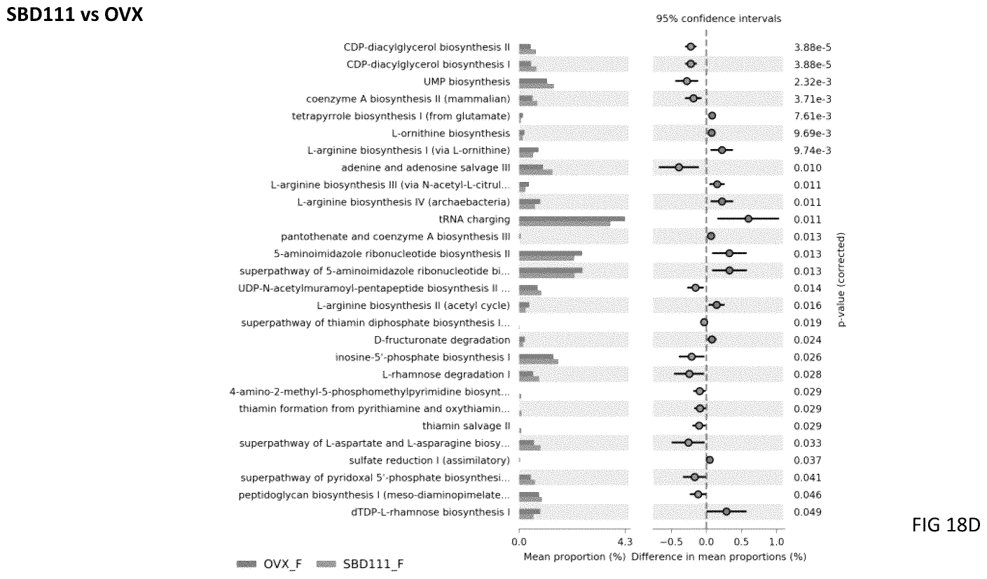

FIG. 15 shows the fragment recruitment of the Bifidobacterium pseudolongum reference genome in the gut from SBD111-treated group at the baseline and week 6 time points. Recruitment plots were built using scripts available at the enveomics toolbox (Rodriguez-R and Konstantinidis 2016).

TABLE 13
Average coverage of B. pseudolongum genome
in the gut metagenomes of mice treated with
SBD111 at the baseline and week 6 time points.

| Subject ID | Baseline | week 6 |
|---|---|---|
| 2266 | 0.95 | 3.19 |
| 2268 | 1.32 | 3.55 |
| 2269 | 1.35 | 2.32 |
| 2270 | 2.7 | 1.37 |
| 2271 | 0.52 | 0.29 |
| 2272 | 0.69 | 3.92 |
| 2273 | 0.37 | 1.75 |
| 2274 | 0.55 | 4.32 |
| 2275 | 2.57 | 1.39 |

The fragment recruitment plot shows that B. pseudolongum was not present in the gut microbiome at the baseline and after 6 weeks in the ovx mouse group since metagenomic reads did not map at any nucleotide identity across the genome sequence (left panel of the plot) with an even coverage.

Opposite, the recruitment plot at the baseline of one of the mice treated with SBD111 shows that there is one B. pseudolongum population in the gut metagenome with genome coverage values of 0.5× and metagenomic reads mapped more than 98% nucleotide identity (dark thick line, top right panel). After 6 weeks, an increase in the abundance of this population was observed (average coverage values of 4.32×) in addition to the increase of discrete populations (light lines in the bottom panel) indicating an increase in the intra-population diversity (reads mapped between 95% and 98% nucleotide identity) of B. pseudolongum in the gut metagenome.

In conclusion, the SDB111-treated group showed an increase in the abundance of B. pseudolongum after 6 weeks (table 12) as well as the diversity of B. pseudolongum in the gut community. Accordingly, the results demonstrate that the administration of a SDB111 resulted in an increase in abundance and diversity of a beneficial microbial population. Bifidobacterium pseudolongum has been shown previously to modulate the immune system and decrease systemic inflammation. Inflammation plays a large role in osteoclastogenesis and the breakdown of bone, so the increased abundance of Bifidobacterium pseudolongum likely decreases systemic inflammatory mediators and thus decreases the resorption of bone, leading to improved BMD and trabecular bone volume in mice treated with SBD111 compared to OVX micc.

### Example 13: Cryopreservant Demonstrating Improved Shelf Life

Cryopreservation was performed using DP53 (Pseudomonas fragi) under conditions using DMSO at 10% or a cryogenic buffer (“Cryobuffer”) at 10% and were compared to PBS as negative control to assess viability at different timepoints after cryo storage at −80° C. A cell pellet containing 1×10\hat{ }9 CFUs/ml measured by colony counts in nutrient agar media were placed in a cryogenic vial and stored at −80° C.

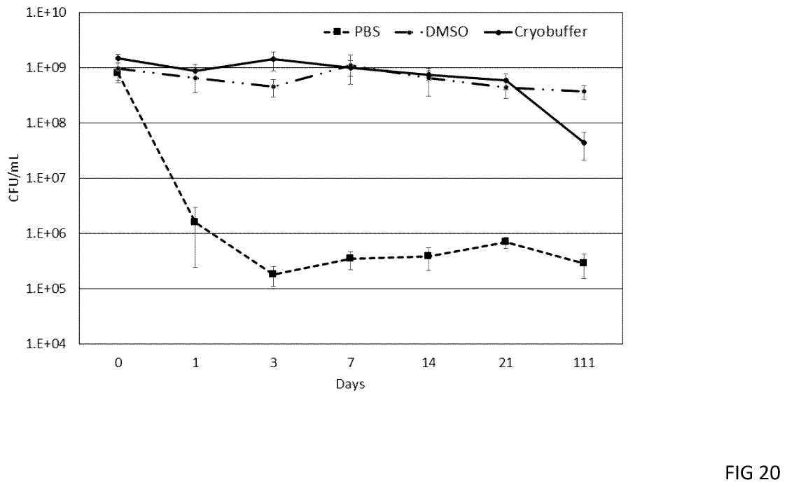

As shown in FIG. 20, the vials with PBS without cryoprotectant showed 1×10\hat{ }6 CFU/ml after 1 day while DMSO and Cryobuffer remained high. After 3 days there was further loss of viability in PBS to 1.75×10\hat{ }5 CFU/ml while DMSO and Cryobuffer maintained the same titer. The vials containing DMSO generally maintained the same titer out to day 111, while the vials containing the Cryobuffer generally maintained the same titer out to day 21 and about a log difference in viability at day 111. Thus, the results demonstrate the use of cryogenic buffer enables cryopreservation and extension of shelf life. Accordingly, the use of cryogenic buffer enables cryopreservation when conducting preclinical or clinical experiments as the product can have the same amount of viable cells at different time points.

### INCORPORATION BY REFERENCE

All references, issued patents, and patent applications cited within the body of the instant specification are hereby incorporated by reference in their entirety, for all purposes. Additionally, Compositions of Oligofructose and Commensal Microorganisms and Methods Thereof, WO2018170034, filed on Mar. 14, 2018 is hereby incorporated by reference.

Sequence Listing

| Seq | | |
|---|---|---|
| ID | | |
| No. | Description | Sequence |
| 1 | DP1 16S rRNA | AGTCAGACATGCAAGTCGAGCGGTAGAGAGAAGCTTGCTTCTCTTGA |
| | | GAGCGGCGGACGGGTGAGTAAAGCCTAGGAATCTGCCTGGTAGTGGG |
| | | GGATAACGTTCGGAAACGGACGCTAATACCGCATACGTCCTACGGGA |
| | | GAAAGCAGGGGACCTTCGGGCCTTGCGCTATCAGATGAGCCTAGGTC |
| | | GGATTAGCTAGTTGGTGAGGTAATGGCTCACCAAGGCGACGATCCGT |
| | | AACTGGTCTGAGAGGATGATCAGTCACACTGGAACTGAGACACGGTC |
| | | CAGACTCCTACGGGAGGCAGCAGTGGGGAATATTGGACAATGGGCGA |
| | | AAGCCTGATCCAGCCATGCCGCGTGTGTGAAGAAGGTCTTCGGATTG |
| | | TAAAGCACTTTAAGTTGGGAGGAAGGGCATTAACCTAATACGTTAGT |
| | | GTTTTGACGTTACCGACAGAATAAGCACCGGCTAACTCTGTGCCAGC |
| | | AGCCGCGGTAATACAGAGGGTGCAAGCGTTAATCGGAATTACTGGGC |
| | | GTAAAGCGCGCGTAGGTGGTTTGTTAAGTTGGATGTGAAATCCCCGG |
| | | GCTCAACCTGGGAACTGCATTCAAAACTGACTGACTAGAGTATGGTA |
| | | GAGGGTGGTGGAATTTCCTGTGTAGCGGTGAAATGCGTAGATATAGG |
| | | AAGGAACACCAGTGGCGAAGGCGACCACCTGGACTAATACTGACACT |
| | | GAGGTGCGAAAGCGTGGGGAGCAAACAGGATTAGATACCCTGGTAGT |
| | | CCACGCCGTAAACGATGTCAACTAGCCGTTGGGAGCCTTGAGCTCTTA |
| | | GTGGCGCAGCTAACGCATTAAGTTGACCGCCTGGGGAGTACGGCCGC |
| | | AAGGTTAAAACTCAAATGAATTGACGGGGGCCCGCACAAGCGGTGGA |
| | | GCATGTGGTTTAATTCGAAGCAACGCGAAGAACCTTACCAGGCCTTG |
| | | ACATCCAATGAACTTTCTAGAGATAGATTGGTGCCTTCGGGAACATTG |
| | | AGACAGGTGCTGCATGGCTGTCGTCAGCTCGTGTCGTGAGATGTTGG |
| | | GTTAAGTCCCGTAACGAGCGCAACCCTTGTCCTTAGTTACCAGCACGT |
| | | AATGGTGGGCACTCTAAGGAGACTGCCGGTGACAAACCGGAGGAAG |
| | | GTGGGGATGACGTCAAGTCATCATGGCCCTTACGGCCTGGGCTACAC |
| | | ACGTGCTACAATGGTCGGTACAGAGGGTTGCCAAGCCGCGAGGTGGA |
| | | GCTAATCCCATAAAACCGATCGTAGTCCGGATCGCAGTCTGCAACTC |
| | | GACTGCGTGAAGTCGGAATCGCTAGTAATCGCGAATCAGAATGTCGC |
| | | GGTGAATACGTTCCCGGGCCTTGTACACACCGCCCGTCACACCATGG |
| | | GAGTGGGTTGCACCAGAAGTAGCTAGTCTAACCTTCGGGAGGACGGT |
| | | TACCACGGTGTGATTCATGACTGGGGTGAAGTCGTAACAAGGTAGCC |
| | | GTAGGGGAACCTGCGGCTGGATCACCTCCTT |
| 2 | DP2 ITS sequence | TTGTTGCTCGAGTTCTTGTTTAGATCTTTTACAATAATGTGTATCTTTA |
| | | ATGAAGATGNGNGCTTAATTGCGCTGCTTTATTAGAGTGTCGCAGTAG |
| | | AAGTAGTCTTGCTTGAATCTCAGTCAACGTTTACACACATTGGAGTTT |
| | | TTTTACTTTAATTTAATTCTTTCTGCTTTGAATCGAAAGGTTCAAGGCA |
| | | AAAAACAAACACAAACAATTTTATTTTATTATAATTTTTTAAACTAAA |
| | | CCAAAATTCCTAACGGAAATTTTAAAATAATTTAAAACTTTCAACAAC |
| | | GGATCTCTTGGTTCTCGCATCGATGAAAAACGTACCGAATTGCGATAA |
| | | GTAATGTGAATTGCAAATACTCGTGAATCATTGAATTTTTGAACGCAC |
| | | ATTGCGCCCTTGAGCATTCTCAAGGGCATGCCTGTTTGAGCGTCATTT |
| | | CCTTCTCAAAAAATAATTTTTTATTTTTTGGTTGTGGGCGATACTCAGG |
| | | GTTAGCTTGAAATTGGAGACTGTTTCAGTCTTTTTTAATTCAACACTTA |
| | | NCTTCTTTGGAGACGCTGTTCTCGCTGTGATGTATTTATGGATTTATTC |
| | | GTTTTACTTTACAAGGGAAATGGTAATGTACCTTAGGCAAAGGGTTGC |
| | | TTTTAATATTCATCAAGTTTGACCTCAAATCAGGTAGGATTACCCGCT |
| | | GAACTTAAGCATATCAATAAGCGGAGGAAAAGAAACCAACTGGGATT |
| | | ACCTTAGTAACGGCGAGTGAAGCGGTAAAAGCTCAAATTTGAAATCT |
| | | GGTACTTTCAGTGCCCGAGTTGTAATTTGTAGAATTTGTCTTTGATTA |
| | | GGTCCTTGTCTATGTTCCTTGGAACAGGACGTCATAGAGGGTGAGANT |
| | | CCCGTTTGNNGAGGATACCTTTTCTCTGTANNACTTTTTCNAAGAGTC |
| | | GAGTTGNTTGGGAATGCAGCTCAAANNGGGTNGNAAATTCCATCTAA |
| | | AGCTAAATATTNGNCNAGAGACCGANAGCGACANTACAGNGATGGA |
| | | AAGANGAAA |
| 3 | DP3 16S rRNA | ATTGAGAGTTTGATCCTGGCTCAGGATGAACGCTGGCGGCGTGCCTA |
| | | ATACATGCAAGTCGAACGCACAGCGAAAGGTGCTTGCACCTTTCAAG |
| | | TGAGTGGCGAACGGGTGAGTAACACGTGGACAACCTGCCTCAAGGCT |
| | | GGGGATAACATTTGGAAACAGATGCTAATACCGAATAAAACTCAGTG |
| | | TCGCATGACACAAAGTTAAAAGGCGCTTTGGCGTCACCTAGAGATGG |
| | | ATCCGCGGTGCATTAGTTAGTTGGTGGGGTAAAGGCCTACCAAGACA |
| | | ATGATGCATAGCCGAGTTGAGAGACTGATCGGCCACATTGGGACTGA |
| | | GACACGGCCCAAACTCCTACGGGAGGCTGCAGTAGGGAATCTTCCAC |
| | | AATGGGCGAAAGCCTGATGGAGCAACGCCGCGTGTGTGATGAAGGCT |
| | | TTCGGGTCGTAAAGCACTGTTGTACGGGAAGAACAGCTAGAATAGGG |
| | | AATGATTTTAGTTTGACGGTACCATACCAGAAAGGGACGGCTAAATA |
| | | CGTGCCAGCAGCCGCGGTAATACGTATGTCCCGAGCGTTATCCGGATT |
| | | TATTGGGCGTAAAGCGAGCGCAGACGGTTGATTAAGTCTGATGTGAA |
| | | AGCCCGGAGCTCAACTCCGGAATGGCATTGGAAACTGGTTAACTTGA |
| | | GTGCAGTAGAGGTAAGTGGAACTCCATGTGTAGCGGTGGAATGCGTA |
| | | GATATATGGAAGAACACCAGTGGCGAAGGCGGCTTACTGGACTGTAA |
| | | CTGACGTTGAGGCTCGAAAGTGTGGGTAGCAAACAGGATTAGATACC |
| | | CTGGTAGTCCACACCGTAAACGATGAACACTAGGTGTTAGGAGGTTT |
| | | CCGCCTCTTAGTGCCGAAGCTAACGCATTAAGTGTTCCGCCTGGGGAG |
| | | TACGACCGCAAGGTTGAAACTCAAAGGAATTGACGGGGACCCGCACA |
| | | AGCGGTGGAGCATGTGGTTTAATTCGAAGCAACGCGAAGAACCTTAC |
| | | CAGGTCTTGACATCCTTTGAAGCTTTTAGAGATAGAAGTGTTCTCTTC |
| | | GGAGACAAAGTGACAGGTGGTGCATGGTCGTCGTCAGCTCGTGTCGT |
| | | GAGATGTTGGGTTAAGTCCCGCAACGAGCGCAACCCTTATTGTTAGTT |
| | | GCCAGCATTCAGATGGGCACTCTAGCGAGACTGCCGGTGACAAACCG |
| | | GAGGAAGGCGGGGACGACGTCAGATCATCATGCCCCTTATGACCTGG |
| | | GCTACACACGTGCTACAATGGCGTATACAACGAGTTGCCAACCCGCG |
| | | AGGGTGAGCTAATCTCTTAAAGTACGTCTCAGTTCGGATTGTAGTCTG |
| | | CAACTCGACTACATGAAGTCGGAATCGCTAGTAATCGCGGATCAGCA |
| | | CGCCGCGGTGAATACGTTCCCGGGTCTTGTACACACCGCCCGTCACAC |
| | | CATGGGAGTTTGTAATGCCCAAAGCCGGTGGCCTAACCTTTTAGGAA |
| | | GGAGCCGTCTAAGGCAGGACAGATGACTGGGGTGAAGTCGTAACAA |
| | | GGTAGCCGTAGGAGAACCTGCGGCTGGATCACCTCCTTT |
| 4 | DP4 16S rRNA | TTGAAGAGTTTGATCATGGCTCAGATTGAACGCTGGCGGCAGGCCTA |
| | | ACACATGCAAGTCGAGCGGCAGCGGAAAGTAGCTTGCTACTTTGCCG |
| | | GCGAGCGGCGGACGGGTGAGTAATGTCTGGGAAACTGCCTGATGGAG |
| | | GGGGATAACTACTGGAAACGGTAGCTAATACCGCATGACCTCGAAAG |
| | | AGCAAAGTGGGGGATCTTCGGACCTCACGCCATCGGATGTGCCCAGA |
| | | TGGGATTAGCTAGTAGGTGAGGTAATGGCTCACCTAGGCGACGATCC |
| | | CTAGCTGGTCTGAGAGGATGACCAGCCACACTGGAACTGAGACACGG |
| | | TCCAGACTCCTACGGGAGGCAGCAGTGGGGAATATTGCACAATGGGC |
| | | GCAAGCCTGATGCAGCCATGCCGCGTGTGTGAAGAAGGCCTTAGGGT |
| | | TGTAAAGCACTTTCAGCGAGGAGGAAGGCATCATACTTAATACGTGT |
| | | GGTGATTGACGTTACTCGCAGAAGAAGCACCGGCTAACTCCGTGCCA |
| | | GCAGCCGCGGTAATACGGAGGGTGCAAGCGTTAATCGGAATTACTGG |
| | | GCGTAAAGCGCACGCAGGCGGTTTGTTAAGTCAGATGTGAAATCCCC |
| | | GCGCTTAACGTGGGAACTGCATTTGAAACTGGCAAGCTAGAGTCTTG |
| | | TAGAGGGGGGTAGAATTCCAGGTGTAGCGGTGAAATGCGTAGAGATC |
| | | TGGAGGAATACCGGTGGCGAAGGCGGCCCCCTGGACAAAGACTGAC |
| | | GCTCAGGTGCGAAAGCGTGGGGAGCAAACAGGATTAGATACCCTGGT |
| | | AGTCCACGCCGTAAACGATGTCGACTTGGAGGTTGTTCCCTTGAGGA |
| | | GTGGCTTCCGGAGCTAACGCGTTAAGTCGACCGCCTGGGGAGTACGG |
| | | CCGCAAGGTTAAAACTCAAATGAATTGACGGGGGCCCGCACAAGCGG |
| | | TGGAGCATGTGGTTTAATTCGATGCAACGCGAAGAACCTTACCTACTC |
| | | TTGACATCCACGGAATTTGGCAGAGATGCCTTAGTGCCTTCGGGAACC |
| | | GTGAGACAGGTGCTGCATGGCTGTCGTCAGCTCGTGTTGTGAAATGTT |
| | | GGGTTAAGTCCCGCAACGAGCGCAACCCTTATCCTTTGTTGCCAGCGA |
| | | TTCGGTCGGGAACTCAAAGGAGACTGCCGGTGATAAACCGGAGGAAG |
| | | GTGGGGATGACGTCAAGTCATCATGGCCCTTACGAGTAGGGCTACAC |
| | | ACGTGCTACAATGGCGCATACAAAGAGAAGCGACCTCGCGAGAGCA |
| | | AGCGGACCTCACAAAGTGCGTCGTAGTCCGGATCGGAGTCTGCAACT |
| | | CGACTCCGTGAAGTCGGAATCGCTAGTAATCGTGGATCAGAATGCCA |
| | | CGGTGAATACGTTCCCGGGCCTTGTACACACCGCCCGTCACACCATGG |
| | | GAGTGGGTTGCAAAAGAAGTAGGTAGCTTAACCTTCGGGAGGGCGCT |
| | | TACCACTTTGTGATTCATGACTGGGGTGAAGTCGTAACAAGGTAACC |
| | | GTAGGGGAACCTGCGGTTGGATCACCTCCTT |
| 5 | DP5 ITS sequence | GCGCTTATTGCGCGGCGAAAAAACCTTACACACAGTGTTTTTTGTTAT |
| | | TACANNAACTTTTGCTTTGGTCTGGACTAGAAATAGTTTGGGCCAGAG |
| | | GTTACTAAACTAAACTTCAATATTTATATTGAATTGTTATTTATTTAAT |
| | | TGTCAATTTGTTGATTAAATTCAAAAAATCTTCAAAACTTTCAACAAC |
| | | GGATCTCTTGGTTCTCGCATCGATGAAGAACGCAGCGAAATGCGATA |
| | | AGTAATATGAATTGCAGATTTTCGTGAATCATCGAATCTTTGAACGCA |
| | | CATTGCGCCCTCTGGTATTCCAGAGGGCATGCCTGTTTGAGCGTCATT |
| | | TCTCTCTCAAACCTTCGGGTTTGGTATTGAGTGATACTCTTAGTCGAA |
| | | CTAGGCGTTTGCTTGAAATGTATTGGCATGAGTGGTACTGGATAGTGC |
| | | TATATGACTTTCAATGTATTAGGTTTATCCAACTCGTTGAATAGTTTA |
| | | ATGGTATATTTCTCGGTATTCTAGGCTCGGCCTTACAATATAACAAAC |
| | | AAGTTTGACCTCAAATCAGGTAGGATTACCCGCTGAACTTAAGCATAT |
| | | CAATAAGCGGAGGAAAAGAAACCAACAGGGATTGCCTTAGTAACGG |
| | | CGAGTGAAGCGGCAAAAGCTCAAATTTGAAATCTGGCACCTTCGGTG |
| | | TCCGAGTTGTAATTTGAAGAAGGTAACTTTGGAGTTGGCTCTTGTCTA |
| | | TGTTCCTTGGAACAGGACGTCACAGAGGGTGAGAATCCCGTGCGATG |
| | | AGATGCCCAATTCTATGTAAAGTGCTTTCGAAGAGTCGAGTTGTTTGG |
| | | GAATGCAGCTCTAAGTGGGTGGTAAATTCCATCTAAAGCTAAATATT |
| | | GGCGAGAGACCGATAGCGAACAAGTACAGTGATGGAAAGATGAAAA |
| | | GAACTTTGAAAAGAGAGTGAAAAAGTACGTGAAATTGTTGAAAGGG |
| | | AAAGGGCTTGAGATCAGACTTGGTATTTTGCGATCCTTTCCTTCTTGG |
| | | TTGGGTTCCTCGCAGCTTACTGGGNCAGCATCGGTTTGGATGG |
| 6 | DP6 16S rRNA | GAAAGGCGGCTTCGGCTGTCACTTATGGATGGACCCGCGTCGCATTA |
| | | GCTAGTTGGTGAGGTAACGGCTCACCAAGGCAACGATGCGTAGCCGA |
| | | CCTGAGAGGGTGATCGGCCACACTGGGACTGAGACACGGCCCAGACT |
| | | CCTACGGGAGGCAGCAGTAGGGAATCTTCCGCAATGGACGAAAGTCT |
| | | GACGGAGCAACGCCGCGTGAGTGATGAAGGCTTTCGGGTCGTAAAAC |
| | | TCTGTTGTTAGGGAAGAACAAGTGCTAGTTGAATAAGCTGCACCTTG |
| | | ACGGTACCTAACCAGAAAGCCACGGCTAACTACGTGCCAGCAGCCGC |
| | | GGTAATACGTAGGTGGCAAGCGTTATCCGGAATTATTGGGCGTAAAG |
| | | CGCGCGCAGGTGGTTTCTTAAGTCTGATGTGAAAGCCCACGGCTCAA |
| | | CCGTGGAGGGTCATTGGAAACTGGGAGACTTGAGTGCAGAAGAGGA |
| | | AAGTGGAATTCCATGTGTAGCGGTGAAATGCGTAGAGATATGGAGGA |
| | | ACACCAGTGGCGAAGGCGACTTTCTGGTCTGTAACTGACACTGAGGC |
| | | GCGAAAGCGTGGGGAGCAAACAGGATTAGATACCCTGGTAGTCCACG |
| | | CCGTAAACGATGAGTGCTAAGTGTTAGAGGGTTTCCGCCCTTTAGTGC |
| | | TGAAGTTAACGCATTAAGCACTCCGCCTGGGGAGTACGGCCGCAAGG |
| | | CTGAAACTCAAAGGAATTGACGGGGGCCCGCACAAGCGGTGGAGCAT |
| | | GTGGTTTAATTCGAAGCAACGCGAAGAACCTTACCAGGTCTTGACAT |
| | | CCTCTGAAAACCCTAGAGATAGGGCTTCTCCTTCGGGAGCAGAGTGA |
| | | CAGGTGGTGCATGGTTGTCGTCAGCTCGTGTCGTGAGATGTTGGGTTA |
| | | AGTCCCGCAACGAGCGCAACCCTTGATCTTAGTTGCCATCATTAAGTT |
| | | GGGCACTCTAAGGTGACTGCCGGTGACAAACCGGAGGAAGGTGGGG |
| | | ATGACGTCAAATCATCATGCCCCTTATGACCTGGGCTACACACGTGCT |
| | | ACAATGGACGGTACAAAGAGCTGCAAGACCGCGAGGTGGAGCTAAT |
| | | CTCATAAAACCGTTCTCAGTTCGGATTGTAGGCTGCAACTCGCCTACA |
| | | TGAAGCTGGAATCGCTAGTAATCGCGGATCAGCAT |
| 7 | DP7 ITS | CCACNCTGCGTGGGCGACACGAAACACCGAAACCGAACGCACGCCGT |
| | | CAAGCAAGAAATCCACAAAACTTTCAACAACGGATCTCTTGGTTCTC |
| | | GCATCGATGAAGAGCGCAGCGAAATGCGATACCTAGTGTGAATTGCA |
| | | GCCATCGTGAATCATCGAGTTCTTGAACGCACATTGCGCCCGCTGGTA |
| | | TTCCGGCGGGCATGCCTGTCTGAGCGTCGTTTCCTTCTTGGAGCGGAG |
| | | CTTCAGACCTGGCGGGCTGTCTTTCGGGACGGCGCGCCCAAAGCGAG |
| | | GGGCCTTCTGCGCGAACTAGACTGTGCGCGCGGGGCGGCCGGCGAAC |
| | | TTATACCAAGCTCGACCTCAGATCAGGCAGGAGTACCCGCTGAACTT |
| | | AAGCATATCAATAAGCGGAGGAAAAGAAACCAACAGGGATTGCCCC |
| | | AGTAGCGGCGAGTGAAGCGGCAAAAGCTCAGATTTGGAATCGCTTCG |
| | | GCGAGTTGTGAATTGCAGGTTGGCGCCTCTGCGGCGGCGGCGGTCCA |
| | | AGTCCCTTGGAACAGGGCGCCATTGAGGGTGAGAGCCCCGTGGGACC |
| | | GTTTGCCTATGCTCTGAGGCCCTTCTGACGAGTCGAGTTGTTTGGGAA |
| | | TGCAGCTCTAAGCGGGTGGTAAATTCCATCTAAGGCTAAATACTGGC |
| | | GAGAGACCGATAGCGAACAAGTACTGTGAAGGAAAGATGAAAAGCA |
| | | CTTTGAAAAGAGAGTGAAACAGCACGTGAAATTGTTGAAAGGGAAG |
| | | GGTATTGCGCCCGACATGGAGCGTGCGCACCGCTGCCCCTCGTGGGC |
| | | GGCGCTCTGGGCGTGCTCTGGGCCAGCATCGGTTTTTGCCGCGGGAG |
| | | AAGGGCGGCGGGCATGTAGCTCTTC |
| 8 | DP8 ITS | GTTGCTCGAGTTCTTGTTTAGATCTTTTACNATAATGTGTATCTTTAAT |
| | | GAAGATGTGCGCTTAATTGCGCTGCTTTATTAGAGTGTCGCAGTAGAA |
| | | GTAGTCTTGCTTGAATCTCAGTCAACGTTTACACACATTGGAGTTTTTT |
| | | TACTTTAATTTAATTCTTTCTGCTTTGAATCGAAAGGTTCAAGGCAAA |
| | | AAACAAACACAAACAATTTTATTTTATTATAATTTTTTAAACTAAACC |
| | | AAAATTCCTAACGGAAATTTTAAAATAATTTAAAACTTTCAACAACG |
| | | GATCTCTTGGTTCTCGCATCGATGAAAAACGTAGCGAATTGCGATAA |
| | | GTAATGTGAATTGCAAATACTCGTGAATCATTGAATTTTTGAACGCAC |
| | | ATTGCGCCCTTGAGCATTCTCAAGGGCATGCCTGTTTGAGCGTCATTT |
| | | CCTTCTCAAAAGATAATTTTTTATTTTTTGGTTGTGGGCGATACTCAGG |
| | | GTTAGCTTGAAATTGGAGACTGTTTCAGTCTTTTTTAATTCAACACTTA |
| | | NCTTCTTTGGAGACGCTGTTCTCGCTGTGATGTATTTATGGATTTATTC |
| | | GTTTTACTTTACAAGGGAAATGGTAATGTACCTTAGGCAAAGGGTTGC |
| | | TTTTAATATTCATCAAGTTTGACCTCAAATCAGGTAGGATTACCCGCT |
| | | GAACTTAAGCATATCAATAAGCGGAGGAAAAGAAACCAACTGGGATT |
| | | ACCTTAGTAACGGCGAGTGAAGCGGTAAAAGCTCAAATTTGAAATCT |
| | | GGTACTTTCANNGCCCGAGTTGTAATTTGTAGAATTTGTCTTTGATTA |
| | | GGTCCTTGTCTATGTTCCTTGGANCAGGACGTCATANAGGGTGANTCC |
| | | CNTTTGGCGANGANACCTTTTCTCTGTANACTTTTTCNANAGTCGAGT |
| | | TGTTTNGGATGCAGCTCNAAGTGGGGNGG |
| 9 | DP9 16S rRNA | ATGAGAGTTTGATCTTGGCTCAGGATGAACGCTGGCGGCGTGCCTAA |
| | | TACATGCAAGTCGAACGAACTTCCGTTAATTGATTATGACGTACTTGT |
| | | ACTGATTGAGATTTTAACACGAAGTGAGTGGCGAACGGGTGAGTAAC |
| | | ACGTGGGTAACCTGCCCAGAAGTAGGGGATAACACCTGGAAACAGAT |
| | | GCTAATACCGTATAACAGAGAAAACCGCATGGTTTTCTTTTAAAAGAT |
| | | GGCTCTGCTATCACTTCTGGATGGACCCGCGGCGTATTAGCTAGTTGG |
| | | TGAGGCAAAGGCTCACCAAGGCAGTGATACGTAGCCGACCTGAGAGG |
| | | GTAATCGGCCACATTGGGACTGAGACACGGCCCAGACTCCTACGGGA |
| | | GGCAGCAGTAGGGAATCTTCCACAATGGACGCAAGTCTGATGGAGCA |
| | | ACGCCGCGTGAGTGAAGAAGGGTTTCGGCTCGTAAAGCTCTGTTGTT |
| | | AAAGAAGAACGTGGGTAAGAGTAACTGTTTACCCAGTGACGGTATTT |
| | | AACCAGAAAGCCACGGCTAACTACGTGCCAGCAGCCGCGGTAATACG |
| | | TAGGTGGCAAGCGTTATCCGGATTTATTGGGCGTAAAGCGAGCGCAG |
| | | GCGGTCTTTTAAGTCTAATGTGAAAGCCTTCGGCTCAACCGAAGAAGT |
| | | GCATTGGAAACTGGGAGACTTGAGTGCAGAAGAGGACAGTGGAACTC |
| | | CATGTGTAGCGGTGAAATGCGTAGATATATGGAAGAACACCAGTGGC |
| | | GAAGGCGGCTGTCTGGTCTGCAACTGACGCTGAGGCTCGAAAGCATG |
| | | GGTAGCGAACAGGATTAGATACCCTGGTAGTCCATGCCGTAAACGAT |
| | | GATTACTAAGTGTTGGAGGGTTTCCGCCCTTCAGTGCTGCAGCTAACG |
| | | CATTAAGTAATCCGCCTGGGGAGTACGACCGCAAGGTTGAAACTCAA |
| | | AAGAATTGACGGGGGCCCGCACAAGCGGTGGAGCATGTGGTTTAATT |
| | | CGAAGCTACGCGAAGAACCTTACCAGGTCTTGACATCTTCTGACAGTC |
| | | TAAGAGATTAGAGGTTCCCTTCGGGGACAGAATGACAGGTGGTGCAT |
| | | GGTTGTCGTCAGCTCGTGTCGTGAGATGTTGGGTTAAGTCCCGCAACG |
| | | AGCGCAACCCTTATTACTAGTTGCCAGCATTAAGTTGGGCACTCTAGT |
| | | GAGACTGCCGGTGACAAACCGGAGGAAGGTGGGGACGACGTCAAAT |
| | | CATCATGCCCCTTATGACCTGGGCTACACACGTGCTACAATGGATGGT |
| | | ACAACGAGTCGCGAGACCGCGAGGTTAAGCTAATCTCTTAAAACCAT |
| | | TCTCAGTTCGGACTGTAGGCTGCAACTCGCCTACACGAAGTCGGAATC |
| | | GCTAGTAATCGCGGATCAGCATGCCGCGGTGAATACGTTCCCGGGCC |
| | | TTGTACACACCGCCCGTCACACCATGAGAGTTTGTAACACCCAAAGC |
| | | CGGTGGGGTAACCTTTTAGGAGCTAGCCGTCTAAGGTGGGACAGATG |
| | | ATTAGGGTGAAGTCGTAACAAGGTAGCCGTAGGAGAACCTGCGGCTG |
| | | GATCACCTCCTT |
| 10 | DP10 16S rRNA | CAGATAGTTGGTGAGGTAACGGCTCACCAAGGCAACGATGCGTAGCC |
| | | GACCTGAGAGGGTGATCGGCCACACTGGGACTGAGACACGGCCCAGA |
| | | CTCCTACGGGAGGCAGCAGTAGGGAATCTTCCGCAATGGACGAAAGT |
| | | CTGACGGAGCAACGCCGCGTGAGTGATGAAGGTTTTCGGATCGTAAA |
| | | GCTCTGTTGTTAGGGAAGAACAAGTGCCGTTCAAATAGGGCGGCACC |
| | | TTGACGGTACCTAACCAGAAAGCCACGGCTAACTACGTGCCAGCAGC |
| | | CGCGGTAATACGTAGGTGGCAAGCGTTGTCCGGAATTATTGGGCGTA |
| | | AAGGGCTCGCAGGCGGTTTCTTAAGTCTGATGTGAAAGCCCCCGGCT |
| | | CAACCGGGGAGGGTCATTGGAAACTGGGGAACTTGAGTGCAGAAGA |
| | | GGAGAGTGGAATTCCACGTGTAGCGGTGAAATGCGTAGAGATGTGGA |
| | | GGAACACCAGTGGCGAAGGCGACTCTCTGGTCTGTAACTGACGCTGA |
| | | GGAGCGAAAGCGTGGGGAGCGAACAGGATTAGATACCCTGGTAGTCC |
| | | ACGCCGTAAACGATGAGTGCTAAGTGTTAGGGGGTTTCCGCCCCTTA |
| | | GTGCTGCAGCTAACGCATTAAGCACTCCGCCTGGGGAGTACGGTCGC |
| | | AAGACTGAAACTCAAAGGAATTGACGGGGGCCCGCACAAGCGGTGG |
| | | AGCATGTGGTTTAATTCGAAGCAACGCGAAGAACCTTACCAGGTCTT |
| | | GACATCCTCTGACAATCCTAGAGATAGGACGTCCCCTTCGGGGGCAG |
| | | AGTGACAGGTGGTGCATGGTTGTCGTCAGCTCGTGTCGTGAGATGTTG |
| | | GGTTAAGTCCCGCAACGAGCGCAACCCTTGATCTTAGTTGCCAGCATT |
| | | CAGTTGGGCACTCTAAGGTGACTGCCGGTGACAAACCGGAGGAAGGT |
| | | GGGGATGACGTCAAATCATCATGCCCCTTATGACCTGGGCTACACAC |
| | | GTGCTACAATGGACAGAACAAAGGGCAGCGAAACCGCGAGGTTAAG |
| | | CCAATCCCACAAATCTGTTCTCAGTTCGGATCGCAGTCTGCAACTCGA |
| | | CTGCGTGAAGCTGGAATCGCTAGTAATCGCGGATCAGCATGCCGCGG |
| | | TGAATACGTTCCCGGGCCTTGTACACACCGCCCGTCACACCACGAGA |
| | | GTTTGTAACACCCGAAGTCGGTGAGGTAACCTTTTAGGAGCCAGCCG |
| | | CCGAAGGTGGGACAGATGATTGGGGTGAAGTCGTAACAAGGTAGCCG |
| | | TATCGGAAGGTGCGGCTGGATCACCTCCTTT |
| 11 | DP11 16S rRNA | TGAAGAGTTTGATCATGGCTCAGATTGAACGCTGGCGGCAGGCCTAA |
| | | CACATGCAAGTCGAGCGGTAGAGAGAAGCTTGCTTCTCTTGAGAGCG |
| | | GCGGACGGGTGAGTAATGCCTAGGAATCTGCCTGGTAGTGGGGGATA |
| | | ACGTTCGGAAACGGACGCTAATACCGCATACGTCCTACGGGAGAAAG |
| | | CAGGGGACCTTCGGGCCTTGCGCTATCAGATGAGCCTAGGTCGGATT |
| | | AGCTAGTTGGTGAGGTAATGGCTCACCAAGGCGACGATCCGTAACTG |
| | | GTCTGAGAGGATGATCAGTCACACTGGAACTGAGACACGGTCCAGAC |
| | | TCCTACGGGAGGCAGCAGTGGGGAATATTGGACAATGGGCGAAAGCC |
| | | TGATCCAGCCATGCCGCGTGTGTGAAGAAGGTCTTCGGATTGTAAAG |
| | | CACTTTAAGTTGGGAGGAAGGGTTGTAGATTAATACTCTGCAATTTTG |
| | | ACGTTACCGACAGAATAAGCACCGGCTAACTCTGTGCCAGCAGCCGC |
| | | GGTAATACAGAGGGTGCAAGCGTTAATCGGAATTACTGGGCGTAAAG |
| | | CGCGCGTAGGTGGTTCGTTAAGTTGGATGTGAAAGCCCCGGGCTCAA |
| | | CCTGGGAACTGCATTCAAAACTGACGAGCTAGAGTATGGTAGAGGGT |
| | | GGTGGAATTTCCTGTGTAGCGGTGAAATGCGTAGATATAGGAAGGAA |
| | | CACCAGTGGCGAAGGCGACCACCTGGACTGATACTGACACTGAGGTG |
| | | CGAAAGCGTGGGGAGCAAACAGGATTAGATACCCTGGTAGTCCACGC |
| | | CGTAAACGATGTCAACTAGCCGTTGGAATCCTTGAGATTTTAGTGGCG |
| | | CAGCTAACGCATTAAGTTGACCGCCTGGGGAGTACGGCCGCAAGGTT |
| | | AAAACTCAAATGAATTGACGGGGGCCCGCACAAGCGGTGGAGCATGT |
| | | GGTTTAATTCGAAGCAACGCGAAGAACCTTACCAGGCCTTGACATCC |
| | | AATGAACTTTCCAGAGATGGATGGGTGCCTTCGGGAACATTGAGACA |
| | | GGTGCTGCATGGCTGTCGTCAGCTCGTGTCGTGAGATGTTGGGTTAAG |
| | | TCCCGTAACGAGCGCAACCCTTGTCCTTAGTTACCAGCACGTTATGGT |
| | | GGGCACTCTAAGGAGACTGCCGGTGACAAACCGGAGGAAGGTGGGG |
| | | ATGACGTCAAGTCATCATGGCCCTTACGGCCTGGGCTACACACGTGCT |
| | | ACAATGGTCGGTACAAAGGGTTGCCAAGCCGCGAGGTGGAGCTAATC |
| | | CCATAAAACCGATCGTAGTCCGGATCGCAGTCTGCAACTCGACTGCG |
| | | TGAAGTCGGAATCGCTAGTAATCGCGAATCAGAATGTCGCGGTGAAT |
| | | ACGTTCCCGGGCCTTGTACACACCGCCCGTCACATCCCACACGAATTG |
| | | CTTG |
| 12 | DP12 16S rRNA | TACGGAGAGTTTGATCCTGGCTCAGGATGAACGCTGGCGGCGTGCTT |
| | | AACACATGCAAGTCGAACGGTGAAGCCAAGCTTGCTTGGTGGATCAG |
| | | TGGCGAACGGGTGAGTAACACGTGAGCAACCTGCCCTGGACTCTGGG |
| | | ATAAGCGCTGGAAACGGCGTCTAATACTGGATATGAGCCTTCATCGC |
| | | ATGGTGGGGGTTGGAAAGATTTTTTGGTCTGGGATGGGCTCGCGGCCT |
| | | ATCAGCTTGTTGGTGAGGTAATGGCTCACCAAGGCGTCGACGGGTAG |
| | | CCGGCCTGAGAGGGTGACCGGCCACACTGGGACTGAGACACGGCCCA |
| | | GACTCCTACGGGAGGCAGCAGTGGGGAATATTGCACAATGGGCGAAA |
| | | GCCTGATGCAGCAACGCCGCGTGAGGGATGACGGCCTTCGGGTTGTA |
| | | AACCTCTTTTAGCAGGGAAGAAGCGAAAGTGACGGTACCTGCAGAAA |
| | | AAGCGCCGGCTAACTACGTGCCAGCAGCCGCGGTAATACGTAGGGCG |
| | | CAAGCGTTATCCGGAATTATTGGGCGTAAAGAGCTCGTAGGCGGTTT |
| | | GTCGCGTCTGCTGTGAAATCCCGAGGCTCAACCTCGGGCCTGCAGTG |
| | | GGTACGGGCAGACTAGAGTGCGGTAGGGGAGATTGGAATTCCTGGTG |
| | | TAGCGGTGGAATGCGCAGATATCAGGAGGAACACCGATGGCGAAGG |
| | | CAGATCTCTGGGCCGTAACTGACGCTGAGGAGCGAAAGGGTGGGGAG |
| | | CAAACAGGCTTAGATACCCTGGTAGTCCACCCCGTAAACGTTGGGAA |
| | | CTAGTTGTGGGGACCATTCCACGGTTTCCGTGACGCAGCTAACGCATT |
| | | AAGTTCCCCGCCTGGGGAGTACGGCCGCAAGGCTAAAACTCAAAGGA |
| | | ATTGACGGGGACCCGCACAAGCGGCGGAGCATGCGGATTAATTCGAT |
| | | GCAACGCGAAGAACCTTACCAAGGCTTGACATACACCAGAACGGGCC |
| | | AGAAATGGTCAACTCTTTGGACACTGGTGAACAGGTGGTGCATGGTT |
| | | GTCGTCAGCTCGTGTCGTGAGATGTTGGGTTAAGTCCCGCAACGAGC |
| | | GCAACCCTCGTTCTATGTTGCCAGCACGTAATGGTGGGAACTCATGGG |
| | | ATACTGCCGGGGTCAACTCGGAGGAAGGTGGGGATGACGTCAAATCA |
| | | TCATGCCCCTTATGTCTTGGGCTTCACGCATGCTACAATGGCCGGTAC |
| | | AAAGGGCTGCAATACCGTGAGGTGGAGCGAATCCCAAAAAGCCGGTC |
| | | CCAGTTCGGATTGAGGTCTGCAACTCGACCTCATGAAGTCGGAGTCG |
| | | CTAGTAATCGCAGATCAGCAACGCTGCGGTGAATACGTTCCCGGGTC |
| | | TTGTACACACCGCCCGTCAAGTCATGAAAGTCGGTAACACCTGAAGC |
| | | CGGTGGCCCAACCCTTGTGGAGGGAGCCGTCGAAGGTGGGATCGGTA |
| | | ATTAGGACTAAGTCGTAACAAGGTAGCCGTACCGGAAGGTGCGGCTG |
| | | GATCACCTCCTTT |
| 13 | DP13 16S rRNA | AGTTAGCGGCGGACGGGTGAGTAACACGTGGGTAACCTGCCTATAAG |
| | | ACTGGGATAACTCCGGGAAACCGGGGCTAATACCGGATAACATTTTG |
| | | CACCGCATGGTGCGAAATTGAAAGGCGGCTTCGGCTGTCACTTATAG |
| | | ATGGACCTGCGGCGCATTAGCTAGTTGGTGAGGTAACGGCTCACCAA |
| | | GGCGACGATGCGTAGCCGACCTGAGAGGGTGATCGGCCACACTGGGA |
| | | CTGAGACACGGCCCAGACTCCTACGGGAGGCAGCAGTAGGGAATCTT |
| | | CCGCAATGGACGAAAGTCTGACGGAGCAACGCCGCGTGAACGATGA |
| | | AGGCTTTCGGGTCGTAAAGTTCTGTTGTTAGGGAAGAACAAGTGCTA |
| | | GTTGAATAAGCTGGCACCTTGACGGTACCTAACCAGAAAGCCACGGC |
| | | TAACTACGTGCCAGCAGCCGCGGTAATACGTAGGTGGCAAGCGTTAT |
| | | CCGGAATTATTGGGCGTAAAGCGCGCGCAGGTGGTTTCTTAAGTCTG |
| | | ATGTGAAAGCCCACGGCTCAACCGTGGAGGGTCATTGGAAACTGGGA |
| | | GACTTGAGTGCAGAAGAGGAAAGTGGAATTCCATGTGTAGCGGTGAA |
| | | ATGCGTAGAGATATGGAGGAACACCAGTGGCGAAGGCGACTTTCTGG |
| | | TCTGCAACTGACACTGAGGCGCGAAAGCGTGGGGAGCAAACAGGATT |
| | | AGATACCCTGGTAGTCCACGCCGTAAACGATGAGTGCTAAGTGTTAG |
| | | AGGGTTTCCGCCCTTTAGTGCTGAAGTTAACGCATTAAGCACTCCGCC |
| | | TGGGGAGTACGGCCGCAAGGCTGAAACTCAAAGGAATTGACGGGGG |
| | | CCCGCACAAGCGGTGGAGCATGTGGTTTAATTCGAAGCAACGCGAAG |
| | | AACCTTACCAGGTCTTGACATCCTCTGAAAACCCTAGAGATAGGGCTT |
| | | CCCCTTCGGGGGCAGAGTGACAGGTGGTGCATGGTTGTCGTCAGCTC |
| | | GTGTCGTGAGATGTTGGGTTAAGTCCCGCAACGAGCGCAACCCTTGA |
| | | TCTTAGTTGCCATCATTAAGTTGGGCACTCTAAGGTGACTGCCGGTGA |
| | | CAAACCGGAGGAAGGTGGGGATGACGTCAAATCATCATGCCCCTTAT |
| | | GACCTGGGCTACACACGTGCTACAATGGACGGTACAAAGAGTCGCAA |
| | | GACCGCGAGGTGGAGCTAATCTCATAAAACCGTTCTCAGTTCGGATT |
| | | GTAGGCTGCAACTCGCCTACATGAAGCTGGAATCGCTAGTAATCGCG |
| | | GATCAGCATGCCGCGGTGAATACGTTCCCGGGCCTTGTACACACCGC |
| | | CCGTCACACCACGAGAGTTTGTAACACCCGAAGTCGGTGGGGTAACC |
| | | TTTTGGAGCCAGCCGCCTAAGGTGGGACAGATGATTGGGGTGAAGTC |
| | | GTAACAAGGTAGCCGTATCGGAAGGTGCGGCTGGATCACCTCCTTT |
| 14 | DP1416S rRNA | TACGGAGAGTTTGATCCTGGCTCAGGATGAACGCTGGCGGCGTGCTT |
| | | AACACATGCAAGTCGAACGATGACTTCTGTGCTTGCACAGAATGATT |
| | | AGTGGCGAACGGGTGAGTAACACGTGAGTAACCTGCCCTTAACTTCG |
| | | GGATAAGCCTGGGAAACCGGGTCTAATACCGGATACGACCTCCTGGC |
| | | GCATGCCATGGTGGTGGAAAGCTTTAGCGGTTTTGGATGGACTCGCG |
| | | GCCTATCAGCTTGTTGGTTGGGGTAATGGCCCACCAAGGCGACGACG |
| | | GGTAGCCGGCCTGAGAGGGTGACCGGCCACACTGGGACTGAGACACG |
| | | GCCCAGACTCCTACGGGAGGCAGCAGTGGGGAATATTGCACAATGGG |
| | | CGAAAGCCTGATGCAGCGACGCCGCGTGAGGGATGACGGCCTTCGGG |
| | | TTGTAAACCTCTTTCAGCAGGGAAGAAGCGAAAGTGACGGTACCTGC |
| | | AGAAGAAGCGCCGGCTAACTACGTGCCAGCAGCCGCGGTAATACGTA |
| | | GGGCGCAAGCGTTATCCGGAATTATTGGGCGTAAAGAGCTCGTAGGC |
| | | GGTTTGTCGCGTCTGCTGTGAAAGCCCGGGGCTCAACCCCGGGTCTGC |
| | | AGTGGGTACGGGCAGACTAGAGTGCAGTAGGGGAGACTGGAATTCCT |
| | | GGTGTAGCGGTGAAATGCGCAGATATCAGGAGGAACACCGATGGCG |
| | | AAGGCAGGTCTCTGGGCTGTAACTGACGCTGAGGAGCGAAAGCATGG |
| | | GGAGCGAACAGGATTAGATACCCTGGTAGTCCATGCCGTAAACGTTG |
| | | GGCACTAGGTGTGGGGGACATTCCACGTTTTCCGCGCCGTAGCTAAC |
| | | GCATTAAGTGCCCCGCCTGGGGAGTACGGCCGCAAGGCTAAAACTCA |
| | | AAGGAATTGACGGGGGCCCGCACAAGCGGCGGAGCATGCGGATTAA |
| | | TTCGATGCAACGCGAAGAACCTTACCAAGGCTTGACATGAACCGGTA |
| | | AGACCTGGAAACAGGTCCCCCACTTGTGGCCGGTTTACAGGTGGTGC |
| | | ATGGTTGTCGTCAGCTCGTGTCGTGAGATGTTGGGTTAAGTCCCGCAA |
| | | CGAGCGCAACCCTCGTTCTATGTTGCCAGCGGGTTATGCCGGGGACTC |
| | | ATAGGAGACTGCCGGGGTCAACTCGGAGGAAGGTGGGGACGACGTC |
| | | AAATCATCATGCCCCTTATGTCTTGGGCTTCACGCATGCTACAATGGC |
| | | CGGTACAAAGGGTTGCGATACTGTGAGGTGGAGCTAATCCCAAAAAG |
| | | CCGGTCTCAGTTCGGATTGAGGTCTGCAACTCGACCTCATGAAGTTGG |
| | | AGTCGCTAGTAATCGCAGATCAGCAACGCTGCGGTGAATACGTTCCC |
| | | GGGCCTTGTACACACCGCCCGTCAAGTCACGAAAGTTGGTAACACCC |
| | | GAAGCCGGTGGCCTAACCCCTTGTGGGAGGGAGCCGTCGAAGGTGGG |
| | | ACCGGCGATTGGGACTAAGTCGTAACAAGGTAGCCGTACCGGAAGGT |
| | | GCGGCTGGATCACCTCCTTT |
| 15 | DP15 16S rRNA | TACGGAGAGTTTGATCCTGGCTCAGGACGAACGCTGGCGGCGTGCTT |
| | | AACACATGCAAGTCGAACGATGATCAGGAGCTTGCTCCTGTGATTAG |
| | | TGGCGAACGGGTGAGTAACACGTGAGTAACCTGCCCCTGACTCTGGG |
| | | ATAAGCGTTGGAAACGACGTCTAATACTGGATATGATCACTGGCCGC |
| | | ATGGTCTGGTGGTGGAAAGATTTTTTGGTTGGGGATGGACTCGCGGCC |
| | | TATCAGCTTGTTGGTGAGGTAATGGCTCACCAAGGCGACGACGGGTA |
| | | GCCGGCCTGAGAGGGTGACCGGCCACACTGGGACTGAGACACGGCCC |
| | | AGACTCCTACGGGAGGCAGCAGTGGGGAATATTGCACAATGGGCGAA |
| | | AGCCTGATGCAGCAACGCCGCGTGAGGGATGACGGCCTTCGGGTTGT |
| | | AAACCTCTTTTAGTAGGGAAGAAGCGAAAGTGACGGTACCTGCAGAA |
| | | AAAGCACCGGCTAACTACGTGCCAGCAGCCGCGGTAATACGTAGGGT |
| | | GCAAGCGTTGTCCGGAATTATTGGGCGTAAAGAGCTCGTAGGCGGTT |
| | | TGTCGCGTCTGCTGTGAAATCCCGAGGCTCAACCTCGGGCTTGCAGTG |
| | | GGTACGGGCAGACTAGAGTGCGGTAGGGGAGATTGGAATTCCTGGTG |
| | | TAGCGGTGGAATGCGCAGATATCAGGAGGAACACCGATGGCGAAGG |
| | | CAGATCTCTGGGCCGTAACTGACGCTGAGGAGCGAAAGCGTGGGGAG |
| | | CGAACAGGATTAGATACCCTGGTAGTCCACGCCGTAAACGTTGGGCG |
| | | CTAGATGTAGGGACCTTTCCACGGTTTCTGTGTCGTAGCTAACGCATT |
| | | AAGCGCCCCGCCTGGGGAGTACGGCCGCAAGGCTAAAACTCAAAGG |
| | | AATTGACGGGGGCCCGCACAAGCGGCGGAGCATGCGGATTAATTCGA |
| | | TGCAACGCGAAGAACCTTACCAAGGCTTGACATACACCGGAAACGGC |
| | | CAGAGATGGTCGCCCCCTTGTGGTCGGTGTACAGGTGGTGCATGGTTG |
| | | TCGTCAGCTCGTGTCGTGAGATGTTGGGTTAAGTCCCGCAACGAGCGC |
| | | AACCCTCGTTCTATGTTGCCAGCGCGTTATGGCGGGGACTCATAGGAG |
| | | ACTGCCGGGGTCAACTCGGAGGAAGGTGGGGATGACGTCAAATCATC |
| | | ATGCCCCTTATGTCTTGGGCTTCACGCATGCTACAATGGCCGGTACAA |
| | | AGGGCTGCGATACCGTAAGGTGGAGCGAATCCCAAAAAGCCGGTCTC |
| | | AGTTCGGATTGAGGTCTGCAACTCGACCTCATGAAGTCGGAGTCGCT |
| | | AGTAATCGCAGATCAGCAACGCTGCGGTGAATACGTTCCCGGGCCTT |
| | | GTACACACCGCCCGTCAAGTCATGAAAGTCGGTAACACCCGAAGCCG |
| | | GTGGCCTAACCCTTGTGGAAGGAGCCGTCGAAGGTGGGATCGGTGAT |
| | | TAGGACTAAGTCGTAACAAGGTAGCCGTACCGGAAGGTGCGGCTGGA |
| | | TCACCTCCTTT |
| 16 | DP16 16S rRNA | GCACTTCATCGTGGTGCACCGTGAAGGGTCTTTGGGCGTTTTACACAT |
| | | GCAAGCAAGTGTTCTATAATTTAGGTTATGGAACAGCCAAATGGTCA |
| | | GTACAGCTCAGTCCTAGGCGATGGACTCCGTAAAACGGGGACAGACT |
| | | ATCCTTTAATAATTAATAGGTTTATTATTTCAATAATAATCTCTAGGA |
| | | AGGGATATACATATATCCTTATTAGTCTAAAGGTTAATAAACCGCCTT |
| | | AGTCAGGACTGAGTTCTCAACAGCTACGGGTTAAACCCCAGGCAACG |
| | | ACGAGTAGGGGATAGTGATAGCTACAACCCCGACACTGGCCGCAAGC |
| | | CAGGGTACTTAAGTACGCAGCAGTGAAGAATCCTCGGCAATGCATCG |
| | | CAATTACCGGTGACCCAATATAAAATAATATCAGGGAGGTAGTAGGT |
| | | GTGACCGGGTGACCCAAAGACGAGTAGTGACATAAGTTATTATTCGC |
| | | GTATGTCGAACATGATAGTGACGTGTTCAACATCAAGCCCCGTCCAA |
| | | CCTCTGTGCCAGCAGTCGCGGTAAAACAGGAGGGGCAGCTCTTATGG |
| | | TCATGAATGGGCGTATAGGGCACGCAGCCAGTTAGTAAAAGCTTGAA |
| | | TATTTATTTTTTTAAAAAGAATGTTTGAGAGGCTATGAGTTTTTATAA |
| | | AGTGTACCCACGACACCAGACTTAGGGCTGAGATCCTATGAAGTCTG |
| | | GGGGCGGTCCTTTAGGGTGCATTGTAAAAACTGACGGTAAGGTGCGA |
| | | CAGCTGGGATACCGAAGCGGAGTAGAGCCCGCCTAGCCCCAGCCGTA |
| | | AACGATAGGGGCCGTTGTTGACTACGGTTTTCAATAAGGCTAACGCCT |
| | | GAGCCCCTCGCCTGTAGGGTATAGCCGCAAGGCCGACATATTAACGA |
| | | TGAGACCGCTGGTGAGCAAACGGGTGCGGGGCATGCTGTTCAATCAG |
| | | ACAGTACGCTGACAACCTTACCACTCCTTGAATCTTTTAGATTATATT |
| | | TCTAAAATGACAGGTGCTGCATGGCCGTCGTCAGTTCGTGGTCGTGAG |
| | | TCGTCCGGTTGAGTCCATGAACGAACGCAGACCCGTCTGTATACTCAG |
| | | TGAAAAGAAATTTAGCTGAACTATACAGTTGTACTTCTATAAAAGGT |
| | | ACCTGTACGGGATTATGACAGGTCGTCATGGCCTTTATGGAGTGGGCT |
| | | ACAGGCGTGCCACACGAGCCGTTTTAACGAGTTCCTCATTTTTATGAA |
| | | TAAGGTCTCTTAATCACGGCTAGTATACGGATCGTAGGCTGTAACTCG |
| | | CCTACGTGAAGTCGGAGTCCCGAGTAATCGCCGATCATCACGCGGCG |
| | | GTGAATCTACACTCTCACTGGGGTACTAACCGCTCGTCACG |
| 17 | DP17 16S rRNA | GTGATTGACGTTACTCGCAGAAGAAGCACCGGCTAACTCCGTGCCAG |
| | | CAGCCGCGGTAATACGGAGGGTGCAAGCGTTAATCGGAATTACTGGG |
| | | CGTAAAGCGCACGCAGGCGGTTTGTTAAGTCAGATGTGAAATCCCCG |
| | | CGCTTAACGTGGGAACTGCATTTGAAACTGGCAAGCTAGAGTCTTGT |
| | | AGAGGGGGGTAGAATTCCAGGTGTAGCGGTGAAATGCGTAGAGATCT |
| | | GGAGGAATACCGGTGGCGAAGGCGGCCCCCTGGACAAAGACTGACG |
| | | CTCAGGTGCGAAAGCGTGGGGAGCAAACAGGATTAGATACCCTGGTA |
| | | GTCCACGCTGTAAACGATGTCGACTTGGAGGTTGTGCCCTTGAGGCGT |
| | | GGCTTCCGGAGCTAACGCGTTAAGTCGACCGCCTGGGGAGTACGGCC |
| | | GCAAGGTTAAAACTCAAATGAATTGACGGGGGCCCGCACAAGCGGTG |
| | | GAGCATGTGGTTTAATTCGATGCAACGCGAAGAACCTTACCTACTCTT |
| | | GACATCCACGGAATTCGCCAGAGATGGCTTAGTGCCTTCGGGAACCG |
| | | TGAGACAGGTGCTGCATGGCTGTCGTCAGCTCGTGTTGTGAAATGTTG |
| | | GGTTAAGTCCCGCAACGAGCGCAACCCTTATCCTTTGTTGCCAGCACG |
| | | TAATGGTGGGAACTCAAAGGAGACTGCCGGTGATAAACCGGAGGAA |
| | | GGTGGGGATGACGTCAAGTCATCATGGCCCTTACGAGTAGGGCTACA |
| | | CACGTGCTACAATGGCATATACAAAGAGAAGCGAACTCGCGAGAGCA |
| | | AGCGGACCTCATAAAGTATGTCGTAGTCCGGATTGGAGTCTGCAACT |
| | | CGACTCCATGAAGTCGGAATCGCTAGTAATCGTAGATCAGAATGCTA |
| | | CGG |
| 18 | DP18 16S rRNA | TGAAGAGTTTGATCATGGCTCAGATTGAACGCTGGCGGCAGGCCTAA |
| | | CACATGCAAGTCGAGCGGATGAAAGGAGCTTGCTCCTGGATTCAGCG |
| | | GCGGACGGGTGAGTAATGCCTAGGAATCTGCCTGGTAGTGGGGGACA |
| | | ACGTTTCGAAAGGAACGCTAATACCGCATACGTCCTACGGGAGAAAG |
| | | CAGGGGACCTTCGGGCCTTGCGCTATCAGATGAGCCTAGGTCGGATT |
| | | AGCTAGTTGGTGAGGTAATGGCTCACCAAGGCGACGATCCGTAACTG |
| | | GTCTGAGAGGATGATCAGTCACACTGGAACTGAGACACGGTCCAGAC |
| | | TCCTACGGGAGGCAGCAGTGGGGAATATTGGACAATGGGCGAAAGCC |
| | | TGATCCAGCCATGCCGCGTGTGTGAAGAAGGTCTTCGGATTGTAAAG |
| | | CACTTTAAGTTGGGAGGAAGGGCAGTAAATTAATACTTTGCTGTTTTG |
| | | ACGTTACCGACAGAATAAGCACCGGCTAACTCTGTGCCAGCAGCCGC |
| | | GGTAATACAGAGGGTGCAAGCGTTAATCGGAATTACTGGGCGTAAAG |
| | | CGCGCGTAGGTGGTTTGTTAAGTTGAATGTGAAATCCCCGGGCTCAAC |
| | | CTGGGAACTGCATCCAAAACTGGCAAGCTAGAGTATGGTAGAGGGTG |
| | | GTGGAATTTCCTGTGTAGCGGTGAAATGCGTAGATATAGGAAGGAAC |
| | | ACCAGTGGCGAAGGCGACCACCTGGACTGATACTGACACTGAGGTGC |
| | | GAAAGCGTGGGGAGCAAACAGGATTAGATACCCTGGTAGTCCACGCC |
| | | GTAAACGATGTCAACTAGCCGTTGGGAGCCTTGAGCTCTTAGTGGCG |
| | | CAGCTAACGCATTAAGTTGACCGCCTGGGGAGTACGGCCGCAAGGTT |
| | | AAAACTCAAATGAATTGACGGGGGCCCGCACAAGCGGTGGAGCATGT |
| | | GGTTTAATTCGAAGCAACGCGAAGAACCTTACCAGGCCTTGACATCC |
| | | AATGAACTTTCCAGAGATGGATTGGTGCCTTCGGGAACATTGAGACA |
| | | GGTGCTGCATGGCTGTCGTCAGCTCGTGTCGTGAGATGTTGGGTTAAG |
| | | TCCCGTAACGAGCGCAACCCTTGTCCTTAGTTACCAGCACGTTATGGT |
| | | GGGCACTCTAAGGAGACTGCCGGTGACAAACCGGAGGAAGGTGGGG |
| | | ATGACGTCAAGTCATCATGGCCCTTACGGCCTGGGCTACACACGTGCT |
| | | ACAATGGTCGGTACAAAGGGTTGCCAAGCCGCGAGGTGGAGCTAATC |
| | | CCATAAAACCGATCGTAGTCCGGATCGCAGTCTGCAACTCGACTGCG |
| | | TGAAGTCGGAATCGCTAGTAATCGCGAATCAGAATGTCGCGGTGAAT |
| | | ACGTTCCCGGGCCTTGTACACACCGCCCGTCACACCATGGGAGTGGG |
| | | TTGCACCAGAAGTAGCTAGTCTAACCTTCGGGAGGACGGTTACCACG |
| | | GTGTGATTCATGACTGGGGTGAAGTCGTAACAAGGTAGCCGTAGGGG |
| | | AACCTGCGGCTGGATCACCTCCTT |
| 19 | DP19 16S rRNA | TACGGAGAGTTTGATCCTGGCTCAGGACGAACGCTGGCGGCGTGCTT |
| | | AACACATGCAAGTCGAACGATGATGCCCAGCTTGCTGGGTGGATTAG |
| | | TGGCGAACGGGTGAGTAACACGTGAGTAACCTGCCCCTGACTCTGGG |
| | | ATAAGCGTTGGAAACGACGTCTAATACTGGATACGACTGCCGGCCGC |
| | | ATGGTCTGGTGGTGGAAAGATTTTTTGGTTGGGGATGGACTCGCGGCC |
| | | TATCAGCTTGTTGGTGAGGTAATGGCTCACCAAGGCGACGACGGGTA |
| | | GCCGGCCTGAGAGGGTGACCGGCCACACTGGGACTGAGACACGGCCC |
| | | AGACTCCTACGGGAGGCAGCAGTGGGGAATATTGCACAATGGGCGAA |
| | | AGCCTGATGCAGCAACGCCGCGTGAGGGATGACGGCCTTCGGGTTGT |
| | | AAACCTCTTTTAGTAGGGAAGAAGCGAAAGTGACGGTACCTGCAGAA |
| | | AAAGCACCGGCTAACTACGTGCCAGCAGCCGCGGTAATACGTAGGGT |
| | | GCAAGCGTTGTCCGGAATTATTGGGCGTAAAGAGCTCGTAGGCGGTT |
| | | TGTCGCGTCTGCTGTGAAATCCCGAGGCTCAACCTCGGGCTTGCAGTG |
| | | GGTACGGGCAGACTAGAGTGCGGTAGGGGAGATTGGAATTCCTGGTG |
| | | TAGCGGTGGAATGCGCAGATATCAGGAGGAACACCGATGGCGAAGG |
| | | CAGATCTCTGGGCCGTAACTGACGCTGAGGAGCGAAAGCGTGGGGAG |
| | | CGAACAGGATTAGATACCCTGGTAGTCCACGCCGTAAACGTTGGGCG |
| | | CTAGATGTAGGGACCTTTCCACGGTTTCTGTGTCGTAGCTAACGCATT |
| | | AAGCGCCCCGCCTGGGGAGTACGGCCGCAAGGCTAAAACTCAAAGG |
| | | AATTGACGGGGGCCCGCACAAGCGGCGGAGCATGCGGATTAATTCGA |
| | | TGCAACGCGAAGAACCTTACCAAGGCTTGACATACACCGGAAACGGC |
| | | CAGAGATGGTCGCCCCCTTGTGGTCGGTGTACAGGTGGTGCATGGTTG |
| | | TCGTCAGCTCGTGTCGTGAGATGTTGGGTTAAGTCCCGCAACGAGCGC |
| | | AACCCTCGTTCTATGTTGCCAGCGCGTTATGGCGGGGACTCATAGGAG |
| | | ACTGCCGGGGTCAACTCGGAGGAAGGTGGGGATGACGTCAAATCATC |
| | | ATGCCCCTTATGTCTTGGGCTTCACGCATGCTACAATGGCCGGTACAA |
| | | AGGGCTGCGATACCGTAAGGTGGAGCGAATCCCAAAAAGCCGGTCTC |
| | | AGTTCGGATTGAGGTCTGCAACTCGACCTCATGAAGTCGGAGTCGCT |
| | | AGTAATCGCAGATCAGCAACGCTGCGGTGAATACGTTCCCGGGCCTT |
| | | GTACACACCGCCCGTCAAGTCATGAAAGTCGGTAACACCCGAAGCCG |
| | | GTGGCCTAACCCTTGTGGAAGGAGCCGTCGAAGGTGGGATCGGTGAT |
| | | TAGGACTAAGTCGTAACAAGGTAGCCGTACCGGAAGGTGCGGCTGGA |
| | | TCACCTCCTTT |
| 20 | DP20 16S rRNA | TGAAGAGTTTGATCCTGGCTCAGAGTGAACGCTGGCGGTAGGCCTAA |
| | | CACATGCAAGTCGAACGGCAGCACAGTAAGAGCTTGCTCTTATGGGT |
| | | GGCGAGTGGCGGACGGGTGAGGAATACATCGGAATCTACCTTTTCGT |
| | | GGGGGATAACGTAGGGAAACTTACGCTAATACCGCATACGACCTTCG |
| | | GGTGAAAGCAGGGGACCTTCGGGCCTTGCGCGGATAGATGAGCCGAT |
| | | GTCGGATTAGCTAGTTGGCGGGGTAAAGGCCCACCAAGGCGACGATC |
| | | CGTAGCTGGTCTGAGAGGATGATCAGCCACACTGGAACTGAGACACG |
| | | GTCCAGACTCCTACGGGAGGCAGCAGTGGGGAATATTGGACAATGGG |
| | | CGCAAGCCTGATCCAGCCATACCGCGTGGGTGAAGAAGGCCTTCGGG |
| | | TTGTAAAGCCCTTTTGTTGGGAAAGAAAAGCAGTCGGCTAATACCCG |
| | | GTTGTTCTGACGGTACCCAAAGAATAAGCACCGGCTAACTTCGTGCC |
| | | AGCAGCCGCGGTAATACGAAGGGTGCAAGCGTTACTCGGAATTACTG |
| | | GGCGTAAAGCGTGCGTAGGTGGTTGTTTAAGTCTGTTGTGAAAGCCCT |
| | | GGGCTCAACCTGGGAATTGCAGTGGATACTGGGCGACTAGAGTGTGG |
| | | TAGAGGGTAGTGGAATTCCCGGTGTAGCAGTGAAATGCGTAGAGATC |
| | | GGGAGGAACATCCATGGCGAAGGCAGCTACCTGGACCAACACTGACA |
| | | CTGAGGCACGAAAGCGTGGGGAGCAAACAGGATTAGATACCCTGGTA |
| | | GTCCACGCCCTAAACGATGCGAACTGGATGTTGGGTGCAATTTGGCA |
| | | CGCAGTATCGAAGCTAACGCGTTAAGTTCGCCGCCTGGGGAGTACGG |
| | | TCGCAAGACTGAAACTCAAAGGAATTGACGGGGGCCCGCACAAGCG |
| | | GTGGAGTATGTGGTTTAATTCGATGCAACGCGAAGAACCTTACCTGGT |
| | | CTTGACATGTCGAGAACTTTCCAGAGATGGATTGGTGCCTTCGGGAAC |
| | | TCGAACACAGGTGCTGCATGGCTGTCGTCAGCTCGTGTCGTGAGATGT |
| | | TGGGTTAAGTCCCGCAACGAGCGCAACCCTTGTCCTTAGTTGCCAGCA |
| | | CGTAATGGTGGGAACTCTAAGGAGACCGCCGGTGACAAACCGGAGG |
| | | AAGGTGGGGATGACGTCAAGTCATCATGGCCCTTACGACCAGGGCTA |
| | | CACACGTACTACAATGGTAGGGACAGAGGGCTGCAAACCCGCGAGG |
| | | GCAAGCCAATCCCAGAAACCCTATCTCAGTCCGGATTGGAGTCTGCA |
| | | ACTCGACTCCATGAAGTCGGAATCGCTAGTAATCGCAGATCAGCATT |
| | | GCTGCGGTGAATACGTTCCCGGGCCTTGTACACACCGCCCGTCACACC |
| | | ATGGGAGTTTGTTGCACCAGAAGCAGGTAGCTTAACCTTCGGGAGGG |
| | | CGCTTGCCACGGTGTGGCCGATGACTGGGGTGAAGTCGTAACAAGGT |
| | | AGCCGTATCGGAAGGTGCGGCTGGATCACCTCCTTT |
| 22 | DP22 16S rRNA | TTGAAGAGTTTGATCATGGCTCAGATTGAACGCTGGCGGCAGGCCTA |
| | | ACACATGCAAGTCGAGCGGCAGCGGGAAGTAGCTTGCTACTTTGCCG |
| | | GCGAGCGGCGGACGGGTGAGTAATGTCTGGGAAACTGCCTGATGGAG |
| | | GGGGATAACTACTGGAAACGGTAGCTAATACCGCATGACCTCGCAAG |
| | | AGCAAAGTGGGGGACCTTCGGGCCTCACGCCATCGGATGTGCCCAGA |
| | | TGGGATTAGCTAGTAGGTGAGGTAATGGCTCACCTAGGCGACGATCC |
| | | CTAGCTGGTCTGAGAGGATGACCAGCCACACTGGAACTGAGACACGG |
| | | TCCAGACTCCTACGGGAGGCAGCAGTGGGGAATATTGCACAATGGGC |
| | | GCAAGCCTGATGCAGCCATGCCGCGTGTGTGAAGAAGGCCTTAGGGT |
| | | TGTAAAGCACTTTCAGCGAGGAGGAAGGGTTCAGTGTTAATAGCACT |
| | | GAACATTGACGTTACTCGCAGAAGAAGCACCGGCTAACTCCGTGCCA |
| | | GCAGCCGCGGTAATACGGAGGGTGCAAGCGTTAATCGGAATTACTGG |
| | | GCGTAAAGCGCACGCAGGCGGTTTGTTAAGTCAGATGTGAAATCCCC |
| | | GAGCTTAACTTGGGAACTGCATTTGAAACTGGCAAGCTAGAGTCTTGT |
| | | AGAGGGGGGTAGAATTCCAGGTGTAGCGGTGAAATGCGTAGAGATCT |
| | | GGAGGAATACCGGTGGCGAAGGCGGCCCCCTGGACAAAGACTGACG |
| | | CTCAGGTGCGAAAGCGTGGGGAGCAAACAGGATTAGATACCCTGGTA |
| | | GTCCACGCTGTAAACGATGTCGACTTGGAGGTTGTGCCCTTGAGGCGT |
| | | GGCTTCCGGAGCTAACGCGTTAAGTCGACCGCCTGGGGAGTACGGCC |
| | | GCAAGGTTAAAACTCAAATGAATTGACGGGGGCCCGCACAAGCGGTG |
| | | GAGCATGTGGTTTAATTCGATGCAACGCGAAGAACCTTACCTACTCTT |
| | | GACATCCAGAGAATTCGCTAGAGATAGCTTAGTGCCTTCGGGAACTC |
| | | TGAGACAGGTGCTGCATGGCTGTCGTCAGCTCGTGTTGTGAAATGTTG |
| | | GGTTAAGTCCCGCAACGAGCGCAACCCTTATCCTTTGTTGCCAGCGAG |
| | | TAATGTCGGGAACTCAAAGGAGACTGCCGGTGATAAACCGGAGGAA |
| | | GGTGGGGATGACGTCAAGTCATCATGGCCCTTACGAGTAGGGCTACA |
| | | CACGTGCTACAATGGCATATACAAAGAGAAGCAAACTCGCGAGAGCA |
| | | AGCGGACCTCATAAAGTATGTCGTAGTCCGGATTGGAGTCTGCAACT |
| | | CGACTCCATGAAGTCGGAATCGCTAGTAATCGTAGATCAGAATGCTA |
| | | CGGTGAATACGTTCCCGGGCCTTGTACACACCGCCCGTCACACCATGG |
| | | GAGTGGGTTGCAAAAGAAGTAGGTAGCTTAACCTTCGGGAGGGCGCT |
| | | TACCACTTTGTGATTCATGACTGGGGTGAAGTCGTAACAAGGTAACC |
| | | GTAGGGGAACCTGCGGTTGGATCACCTCCTT |
| 23 | DP23 16S rRNA | TTGAAGAGTTTGATCATGGCTCAGATTGAACGCTGGCGGCAGGCCTA |
| | | ACACATGCAAGTCGAACGGTAGCACAGAGAGCTTGCTCTTGGGTGAC |
| | | GAGTGGCGGACGGGTGAGTAATGTCTGGGAAACTGCCCGATGGAGGG |
| | | GGATAACTACTGGAAACGGTAGCTAATACCGCATAACGTCTTCGGAC |
| | | CAAAGTGGGGGACCTTCGGGCCTCACACCATCGGATGTGCCCAGATG |
| | | GGATTAGCTAGTAGGTGGGGTAATGGCTCACCTAGGCGACGATCCCT |
| | | AGCTGGTCTGAGAGGATGACCAGCCACACTGGAACTGAGACACGGTC |
| | | CAGACTCCTACGGGAGGCAGCAGTGGGGAATATTGCACAATGGGCGC |
| | | AAGCCTGATGCAGCCATGCCGCGTGTATGAAGAAGGCCTTCGGGTTG |
| | | TAAAGTACTTTCAGCGGGGAGGAAGGCGATACGGTTAATAACCGTGT |
| | | CGATTGACGTTACCCGCAGAAGAAGCACCGGCTAACTCCGTGCCAGC |
| | | AGCCGCGGTAATACGGAGGGTGCAAGCGTTAATCGGAATTACTGGGC |
| | | GTAAAGCGCACGCAGGCGGTCTGTCAAGTCAGATGTGAAATCCCCGG |
| | | GCTTAACCTGGGAACTGCATTTGAAACTGGCAGGCTTGAGTCTCGTAG |
| | | AGGGGGGTAGAATTCCAGGTGTAGCGGTGAAATGCGTAGAGATCTGG |
| | | AGGAATACCGGTGGCGAAGGCGGCCCCCTGGACGAAGACTGACGCTC |
| | | AGGTGCGAAAGCGTGGGGAGCAAACAGGATTAGATACCCTGGTAGTC |
| | | CACGCTGTAAACGATGTCGACTTGGAGGTTGTGCCCTTGAGGCGTGG |
| | | CTTCCGGAGCTAACGCGTTAAGTCGACCGCCTGGGGAGTACGGCCGC |
| | | AAGGTTAAAACTCAAATGAATTGACGGGGGCCCGCACAAGCGGTGGA |
| | | GCATGTGGTTTAATTCGATGCAACGCGAAGAACCTTACCTGGCCTTGA |
| | | CATCCACAGAATTCGGCAGAGATGCCTTAGTGCCTTCGGGAACTGTG |
| | | AGACAGGTGCTGCATGGCTGTCGTCAGCTCGTGTTGTGAAATGTTGGG |
| | | TTAAGTCCCGCAACGAGCGCAACCCTTATCCTTTGTTGCCAGCGATTC |
| | | GGTCGGGAACTCAAAGGAGACTGCCGGTGATAAACCGGAGGAAGGT |
| | | GGGGATGACGTCAAGTCATCATGGCCCTTACGGCCAGGGCTACACAC |
| | | GTGCTACAATGGCGCATACAAAGAGAAGCGACCTCGCGAGAGCAAG |
| | | CGGACCTCATAAAGTGCGTCGTAGTCCGGATCGGAGTCTGCAACTCG |
| | | ACTCCGTGAAGTCGGAATCGCTAGTAATCGTAGATCAGAATGCTACG |
| | | GTGAATACGTTCCCGGGCCTTGTACACACCGCCCGTCACACCATGGG |
| | | AGTGGGTTGCAAAAGAAGTAGGTAGCTTAACCTTCGGGAGGGCGCTT |
| | | ACCACTTTGTGATTCATGACTGGGGTGAAGTCGTAACAAGGTAACCG |
| | | TAGGGGAACCTGCGGTTGGATCACCTCCTT |
| 24 | DP24 16S rRNA | AGCATTTGATTATGGTGCTTACTGATTGCTATCTAGGGGTTTAACACA |
| | | TGCTAGTCAATGATCTTTTAGATTATGGCGTACGGGCTAGGAATACTT |
| | | AGAATGATAACTCTATGATCGCAGTAATAGCGTAAAAGGTATAATAC |
| | | CGCATAGAGGTTCGCTTCGTATCTCAATAGGTAGTTGGTGAGGTAAA |
| | | GCTCAACAAGCCGATGATGAGTAATATTGGATGAAAGTCTTAAATAT |
| | | AGCAGTGGAAATGAAAAAGTCCACCGTTATTTATTAACGCAGCAGTG |
| | | GAGAATCGTCGTAATGTGCAGTATTCATTTATGGATAAGCATGAACG |
| | | CGCTACCTAGATTCGGATAGGAGATAGCATCTTCTACCGATAAAAGA |
| | | ACTTAGAATAATGATCTAGTTCTCATTAGTGGGTGACAATCGCCGTGC |
| | | CAGCATCAGCGGTAAAACGGCTTCCGCAAGCAATAGTAATTTAAATT |
| | | GGTGTAAAGGGTACGTAGCCGGCCTTATTAGGCTAGAGTTAGATACG |
| | | GGTAAGTACAATACTTGGAGTAGGGCTGATATCTTATGATCCCAAGG |
| | | GGAGTGCTAAAGGCGAAGGCAACTTACTGGTAATAACTGACGGTGAG |
| | | GTACGAAGGTCAGGGCATGGAAAGAGATTAGATACCTCATTACTCCT |
| | | GACAGTAAACGATGTAGATTAAAGATTGGAATAATTCTGTCTTAACG |
| | | CTAACGCATTAAATCTACCACCTGTAGAGTATAGTCGCAAGGCCGAA |
| | | ATACAAATAATTAGACGGCTCTAGAGCAAACGGAGTGAAGCATGTTA |
| | | TTTAATACGATAACCCGCGTAAAATCTTACCAGTTCTTGAATCTTAGA |
| | | CAGGTGTTGCATGGTTGTCGTCAGCTCGTGCTAATGGTGTCTGGTTAA |
| | | TTCCAAATAACGAGCGCAATCCTTACTTCTAGTTTTCTAGGAGTCTCC |
| | | ATTTGACATACGTGTCAATGGTTTAAGGAATATGACAAACCCTCATGG |
| | | CCCTTATGGACTGGGCAATAGACGTGCCACAAGAATCTAGACAAAAT |
| | | GACGCGAAATGGTAACAATGAGCTAATCATCAAAGAAGATTAATGTA |
| | | CGAATTATGGGCTGGAACTCGCCCATATGAAGTAGGAATTCCGAGTA |
| | | ATCGCGTATCAGAACGACGCGGTGAACATCATCTCTGGAGTGTACTA |
| | | ACTGCTCGTCACGGGACGAAAGGGAGTGTATTATGAAGTGGGGCTAA |
| | | TTGGTTAACTCCGGTGAGTGTCACGAATAATCCTTCCCGATTGTTCTG |
| | | AAGTCGAAACAAGGTAACCGTAAGGGAACTTGCGGTTGA |
| 25 | DP25 16S rRNA | TACGGAGAGTTTGATCCTGGCTCAGGATGAACGCTGGCGGCGTGCTT |
| | | AACACATGCAAGTCGAACGGTGAAGCCAAGCTTGCTTGGTGGATCAG |
| | | TGGCGAACGGGTGAGTAACACGTGAGCAACCTGCCCTGGACTCTGGG |
| | | ATAAGCGCTGGAAACGGCGTCTAATACTGGATATGAGCTCCTTCCGC |
| | | ATGGTGGGGGTTGGAAAGATTTTTCGGTCTGGGATGGGCTCGCGGCC |
| | | TATCAGCTTGTTGGTGAGGTAATGGCTCACCAAGGCGTCGACGGGTA |
| | | GCCGGCCTGAGAGGGTGACCGGCCACACTGGGACTGAGACACGGCCC |
| | | AGACTCCTACGGGAGGCAGCAGTGGGGAATATTGCACAATGGGCGGA |
| | | AGCCTGATGCAGCAACGCCGCGTGAGGGATGACGGCCTTCGGGTTGT |
| | | AAACCTCTTTTAGCAGGGAAGAAGCGAAAGTGACGGTACCTGCAGAA |
| | | AAAGCGCCGGCTAACTACGTGCCAGCAGCCGCGGTAATACGTAGGGC |
| | | GCAAGCGTTATCCGGAATTATTGGGCGTAAAGAGCTCGTAGGCGGTT |
| | | TGTCGCGTCTGCTGTGAAATCCCGAGGCTCAACCTCGGGCCTGCAGTG |
| | | GGTACGGGCAGACTAGAGTGCGGTAGGGGAGATTGGAATTCCTGGTG |
| | | TAGCGGTGGAATGCGCAGATATCAGGAGGAACACCGATGGCGAAGG |
| | | CAGATCTCTGGGCCGTAACTGACGCTGAGGAGCGAAAGGGTGGGGAG |
| | | CAAACAGGCTTAGATACCCTGGTAGTCCACCCCGTAAACGTTGGGAA |
| | | CTAGTTGTGGGGACCATTCCACGGTTTCCGTGACGCAGCTAACGCATT |
| | | AAGTTCCCCGCCTGGGGAGTACGGCCGCAAGGCTAAAACTCAAAGGA |
| | | ATTGACGGGGACCCGCACAAGCGGCGGAGCATGCGGATTAATTCGAT |
| | | GCAACGCGAAGAACCTTACCAAGGCTTGACATATACGAGAACGGGCC |
| | | AGAAATGGTCAACTCTTTGGACACTCGTAAACAGGTGGTGCATGGTT |
| | | GTCGTCAGCTCGTGTCGTGAGATGTTGGGTTAAGTCCCGCAACGAGC |
| | | GCAACCCTCGTTCTATGTTGCCAGCACGTAATGGTGGGAACTCATGGG |
| | | ATACTGCCGGGGTCAACTCGGAGGAAGGTGGGGATGACGTCAAATCA |
| | | TCATGCCCCTTATGTCTTGGGCTTCACGCATGCTACAATGGCCGGTAC |
| | | AAAGGGCTGCAATACCGTAAGGTGGAGCGAATCCCAAAAAGCCGGTC |
| | | CCAGTTCGGATTGAGGTCTGCAACTCGACCTCATGAAGTCGGAGTCG |
| | | CTAGTAATCGCAGATCAGCAACGCTGCGGTGAATACGTTCCCGGGTC |
| | | TTGTACACACCGCCCGTCAAGTCATGAAAGTCGGTAACACCTGAAGC |
| | | CGGTGGCCCAACCCTTGTGGAGGGAGCCGTCGAAGGTGGGATCGGTA |
| | | ATTAGGACTAAGTCGTAACAAGGTAGCCGTACCGGAAGGTGCGGCTG |
| | | GATCACCTCCTTT |
| 26 | DP26 16S rRNA | CTTGAGAGTTTGATCCTGGCTCAGAGCGAACGCTGGCGGCAGGCTTA |
| | | ACACATGCAAGTCGAGCGGGCATCTTCGGATGTCAGCGGCAGACGGG |
| | | TGAGTAACACGTGGGAACGTACCCTTCGGTTCGGAATAACGCTGGGA |
| | | AACTAGCGCTAATACCGGATACGCCCTTTTGGGGAAAGGTTTACTGCC |
| | | GAAGGATCGGCCCGCGTCTGATTAGCTAGTTGGTGGGGTAACGGCCT |
| | | ACCAAGGCGACGATCAGTAGCTGGTCTGAGAGGATGATCAGCCACAC |
| | | TGGGACTGAGACACGGCCCAGACTCCTACGGGAGGCAGCAGTGGGG |
| | | AATATTGGACAATGGGCGCAAGCCTGATCCAGCCATGCCGCGTGAGT |
| | | GATGAAGGCCTTAGGGTTGTAAAGCTCTTTTGTCCGGGACGATAATG |
| | | ACGGTACCGGAAGAATAAGCCCCGGCTAACTTCGTGCCAGCAGCCGC |
| | | GGTAATACGAAGGGGGCTAGCGTTGCTCGGAATCACTGGGCGTAAAG |
| | | GGCGCGTAGGCGGCCATTCAAGTCGGGGGTGAAAGCCTGTGGCTCAA |
| | | CCACAGAATTGCCTTCGATACTGTTTGGCTTGAGTATGGTAGAGGTTG |
| | | GTGGAACTGCGAGTGTAGAGGTGAAATTCGTAGATATTCGCAAGAAC |
| | | ACCGGTGGCGAAGGCGGCCAACTGGACCATTACTGACGCTGAGGCGC |
| | | GAAAGCGTGGGGAGCAAACAGGATTAGATACCCTGGTAGTCCACGCC |
| | | GTAAACGATGAATGCCAGCTGTTGGGGTGCTTGCACCTCAGTAGCGC |
| | | AGCTAACGCTTTAAGCATTCCGCCTGGGGAGTACGGTCGCAAGATTA |
| | | AAACTCAAAGGAATTGACGGGGGCCCGCACAAGCGGTGGAGCATGT |
| | | GGTTTAATTCGAAGCAACGCGCAGAACCTTACCATCCCTTGACATGGC |
| | | ATGTTACCCGGAGAGATTCGGGGTCCACTTCGGTGGCGTGCACACAG |
| | | GTGCTGCATGGCTGTCGTCAGCTCGTGTCGTGAGATGTTGGGTTAAGT |
| | | CCCGCAACGAGCGCAACCCACGTCCTTAGTTGCCATCATTCAGTTGGG |
| | | CACTCTAGGGAGACTGCCGGTGATAAGCCGCGAGGAAGGTGTGGATG |
| | | ACGTCAAGTCCTCATGGCCCTTACGGGATGGGCTACACACGTGCTAC |
| | | AATGGCGGTGACAGTGGGACGCGAAGGAGCGATCTGGAGCAAATCC |
| | | CCAAAAACCGTCTCAGTTCAGATTGCACTCTGCAACTCGAGTGCATGA |
| | | AGGCGGAATCGCTAGTAATCGTGGATCAGCATGCCACGGTGAATACG |
| | | TTCCCGGGCCTTGTACACACCGCCCGTCACACCATGGGAGTTGGTCTT |
| | | ACCCGACGGCGCTGCGCCAACCGCAAGGAGGCAGGCGACCACGGTA |
| | | GGGTCAGCGACTGGGGTGAAGTCGTAACAAGGTAGCCGTAGGGGAA |
| | | CCTGCGGCTGGATCACCTCCTTT |
| 27 | DP27 16S rRNA | CTTGAGAGTTTGATCCTGGCTCAGAACGAACGCTGGCGGCATGCCTA |
| | | ACACATGCAAGTCGAACGATGCTTTCGGGCATAGTGGCGCACGGGTG |
| | | CGTAACGCGTGGGAATCTGCCCTCAGGTTCGGAATAACAGCTGGAAA |
| | | CGGCTGCTAATACCGGATGATATCGCAAGATCAAAGATTTATCGCCT |
| | | GAGGATGAGCCCGCGTTGGATTAGGTAGTTGGTGGGGTAAAGGCCTA |
| | | CCAAGCCGACGATCCATAGCTGGTCTGAGAGGATGATCAGCCACACT |
| | | GGGACTGAGACACGGCCCAGACTCCTACGGGAGGCAGCAGTGGGGA |
| | | ATATTGGACAATGGGCGCAAGCCTGATCCAGCAATGCCGCGTGAGTG |
| | | ATGAAGGCCCTAGGGTTGTAAAGCTCTTTTACCCGGGAAGATAATGA |
| | | CTGTACCGGGAGAATAAGCCCCGGCTAACTCCGTGCCAGCAGCCGCG |
| | | GTAATACGGAGGGGGCTAGCGTTGTTCGGAATTACTGGGCGTAAAGC |
| | | GCACGTAGGCGGCTTTGTAAGTCAGAGGTGAAAGCCTGGAGCTCAAC |
| | | TCCAGAACTGCCTTTGAGACTGCATCGCTTGAATCCAGGAGAGGTCA |
| | | GTGGAATTCCGAGTGTAGAGGTGAAATTCGTAGATATTCGGAAGAAC |
| | | ACCAGTGGCGAAGGCGGCTGACTGGACTGGTATTGACGCTGAGGTGC |
| | | GAAAGCGTGGGGAGCAAACAGGATTAGATACCCTGGTAGTCCACGCC |
| | | GTAAACGATGATAACTAGCTGTCCGGGCACTTGGTGCTTGGGTGGCG |
| | | CAGCTAACGCATTAAGTTATCCGCCTGGGGAGTACGGCCGCAAGGTT |
| | | AAAACTCAAAGGAATTGACGGGGGCCTGCACAAGCGGTGGAGCATGT |
| | | GGTTTAATTCGAAGCAACGCGCAGAACCTTACCAGCGTTTGAC |
| 28 | DP28 16S rRNA | ATAGTCGGGGGCATCAGTATTCAATTGTCAGAGGTGAAATTCTTGGAT |
| | | TTATTGAAGACTAACTACTGCGAAAGCATTTGCCAAGGATGTTTTCAT |
| | | TAATCAGTGAACGAAAGTTAGGGGATCGAAGACGATCAGATACCGTC |
| | | GTAGTCTTAACCATAAACTATGCCGACTAGGGATCGGGCGATGTTATC |
| | | ATTTTGACTCGCTCGGCACCTTACGAGAAATCAAAGTCTTTGGGTTCT |
| | | GGGGGGAGTATGGTCGCAAGGCTGAAACTTAAAGAAATTGACGGAA |
| | | GGGCACCACCAGGCGTGGAGCCTGCGGCTTAATTTGACTCAACACGG |
| | | GGAAACTCACCAGGTCCAGACACAATAAGGATTGACAGATTGAGAGC |
| | | TCTTTCTTGATTTTGTGGGTGGTGGTGCATGGCCGTTCTTAGTTGGTGG |
| | | AGTGATTTGTCTGCTTAATTGCGATAACGAACGAGACCTTAACCTGCT |
| | | AAATAGCCCGGCCCGCTTTGGCGGGTCGCCGGCTTCTTAGAGGGACT |
| | | ATCGGCTCAAGCCGATGGAAGTTTGAGGCAATAACAGGTCTGTGATG |
| | | CCCTTAGATGTTCTGGGCCGCACGCGCGCTACACTGACAGAGCCAAC |
| | | GAGTTCATTTCCTTGCCCGGAAGGGTTGGGTAATCTTGTTAAACTCTG |
| | | TCGTGCTGGGGATAGAGCATTGCAATTATTGCTCTTCAACGAGGAATG |
| | | CCTAGTAAGCGTACGTCATCAGCGTGCGTTGATTACGTCCCTGCCCTT |
| | | TGTACACACCGCCCGTCGCTACTACCGATTGAATGGCTGAGTGAGGC |
| | | CTTCGGACTGGCCCAGGGAGGTCGGCAACGACCACCCAGGGCCGGAA |
| | | AGTTGGTCAAACTCCGTCATTTAGAGGAAGTAAAAGTCGTAACAAGG |
| | | TTTCCGTAGGTGAACCTGCGGAAGGATCA |
| 29 | DP29 16S rRNA | TACGGAGAGTTTGATCCTGGCTCAGGACGAACGCTGGCGGCGTGCTT |
| | | AACACATGCAAGTCGAACGATGAAGCCCAGCTTGCTGGGTTGATTAG |
| | | TGGCGAACGGGTGAGTAACACGTGAGCAACGTGCCCATAACTCTGGG |
| | | ATAACCTCCGGAAACGGTGGCTAATACTGGATATCTAACACGATCGC |
| | | ATGGTCTGTGTTTGGAAAGATTTTTTGGTTATGGATCGGCTCACGGCC |
| | | TATCAGCTTGTTGGTGAGGTAATGGCTCACCAAGGCGACGACGGGTA |
| | | GCCGGCCTGAGAGGGTGACCGGCCACACTGGGACTGAGACACGGCCC |
| | | AGACTCCTACGGGAGGCAGCAGTGGGGAATATTGCACAATGGGCGAA |
| | | AGCCTGATGCAGCAACGCCGCGTGAGGGATGACGGCATTCGGGTTGT |
| | | AAACCTCTTTTAGTAGGGAAGAAGCGAAAGTGACGGTACCTGCAGAA |
| | | AAAGCACCGGCTAACTACGTGCCAGCAGCCGCTGTAATACGTAGGGT |
| | | GCAAGCGTTGTCCGGAATTATTGGGCGTAAAGAGCTCGTAGGCGGTT |
| | | TGTCGCGTCTGCTGTGAAATCCCGAGGCTCAACCTCGGGTCTGCAGTG |
| | | GGTACGGGCAGACTAGAGTGTGGTAGGGGAGATTGGAATTCCTGGTG |
| | | TAGCGGTGGAATGCGCAGATATCAGGAGGAACACCGATGGCGAAGG |
| | | CAGATCTCTGGGCCATTACTGACGCTGAGGAGCGAAAGCATGGGGAG |
| | | CGAACAGGATTAGATACCCTGGTAGTCCATGCCGTAAACGTTGGGCG |
| | | CTAGATGTGGGGACCATTCCACGGTTTCCGTGTCGTAGCTAACGCATT |
| | | AAGCGCCCCGCCTGGGGAGTACGGCCGCAAGGCTAAAACTCAAAGG |
| | | AATTGACGGGGGCCCGCACAAGCGGCGGAGCATGCGGATTAATTCGA |
| | | TGCAACGCGAAGAACCTTACCAAGGCTTGACATATACCGGAAACGTT |
| | | CAGAAATGTTCGCC |
| 30 | DP30 16S rRNA | TACGGAGAGTTTGATCCTGGCTCAGGATGAACGCTGGCGGCGTGCTT |
| | | AACACATGCAAGTCGAACGGTGAAGCCAAGCTTGCTTGGTGGATCAG |
| | | TGGCGAACGGGTGAGTAACACGTGAGCAACCTGCCCTGGACTCTGGG |
| | | ATAAGCGCTGGAAACGGCGTCTAATACTGGATATGAGACGTGATCGC |
| | | ATGGTCGTGTTTGGAAAGATTTTTCGGTCTGGGATGGGCTCGCGGCCT |
| | | ATCAGCTTGTTGGTGAGGTAATGGCTCACCAAGGCGTCGACGGGTAG |
| | | CCGGCCTGAGAGGGTGACCGGCCACACTGGGACTGAGACACGGCCCA |
| | | GACTCCTACGGGAGGCAGCAGTGGGGAATATTGCACAATGGGCGAAA |
| | | GCCTGATGCAGCAACGCCGCGTGAGGGATGACGGCCTTCGGGTTGTA |
| | | AACCTCTTTTAGCAGGGAAGAAGCGAAAGTGACGGTACCTGCAGAAA |
| | | AAGCGCCGGCTAACTACGTGCCAGCAGCCGCGGTAATACGTAGGGCG |
| | | CAAGCGTTATCCGGAATTATTGGGCGTAAAGAGCTCGTAGGCGGTTT |
| | | GTCGCGTCTGCTGTGAAATCCCGAGGCTCAACCTCGGGCCTGCAGTG |
| | | GGTACGGGCAGACTAGAGTGCGGTAGGGGAGATTGGAATTCCTGGTG |
| | | TAGCGGTGGAATGCGCAGATATCAGGAGGAACACCGATGGCGAAGG |
| | | CAGATCTCTGGGCCGTAACTGACGCTGAGGAGCGAAAGGGTGGGGAG |
| | | CAAACAGGCTTAGATACCCTGGTAGTCCACCCCGTAAACGTTGGGAA |
| | | CTAGTTGTGGGGACCATTCCACGGTTTCCGTGACGCAGCTAACGCATT |
| | | AAGTTCCCCGCCTGGGGAGTACGGCCGCAAGGCTAAAACTCAAAGGA |
| | | ATTGACGGGGACCCGCACAAGCGGCGGAGCATGCGGATTAATTCGAT |
| | | GCAACGCGAAGAACCTTACCAAGGCTTGACATATACGAGAACGGGCC |
| | | AGAAATGGTCAACTCTTTGGACACTCGTAAACAGGTGGTGCATGGTT |
| | | GTCGTCAGCTCGTGTCGTGAGATGTTGGGTTAAGTCCCGCAACGAGC |
| | | GCAACCCTCGTTCTATGTTGCCAGCACGTAATGGTGGGAACTCATGGG |
| | | ATACTGCCGGGGTCAACTCGGAGGAAGGTGGGGATGACGTCAAATCA |
| | | TCATGCCCCTTATGTCTTGGGCTTCACGCATGCTACAATGGCCGGTAC |
| | | AAAGGGCTGCAATACCGTGAGGTGGAGCGAATCCCAAAAAGCCGGTC |
| | | CCAGTTCGGATTGAGGTCTGCAACTCGACCTCATGAAGTCGGAGTCG |
| | | CTAGTAATCGCAGATCAGCAACGCTGCGGTGAATACGTTCCCGGGTC |
| | | TTGTACACACCGCCCGTCAAGTCATGAAAGTCGGTAACACCTGAAGC |
| | | CGGTGGCCCAACCCTTGTGGAGGGAGCCGTCGAAGGTGGGATCGGTA |
| | | ATTAGGACTAAGTCGTAACAAGGTAGCCGTACCGGAAGGTGCGGCTG |
| | | GATCACCTCCTTT |
| 31 | DP31 16SrRNA | CAGCCGGGGGCATTAGTATTTGCACGCTAGAGGTGAAATTCTTGGATT |
| | | GTGCAAAGACTTCCTACTGCGAAAGCATTTGCCAAGAATGTTTTCATT |
| | | AATCAAGAACGAAGGTTAGGGTATCGAAAACGATTAGATACCGTTGT |
| | | AGTCTTAACAGTAAACTATGCCGACTCCGAATCGGTCGATGCTCATTT |
| | | CACTGGCTCGATCGGCGCGGTACGAGAAATCAAAGTTTTTGGGTTCTG |
| | | GGGGGAGTATGGTCGCAAGGCTGAAACTTAAAGAAATTGACGGAAG |
| | | GGCACCACCAGGAGTGGAGCCTGCGGCTTAATTTGACTCAACACGGG |
| | | AAAACTCACCGGGTCCGGACATAGTAAGGATTGACAGATTGATGGCG |
| | | CTTTCATGATTCTATGGGTGGTGGTGCATGGCCGTTCTTAGTTGGTGG |
| | | AGTGATTTGTCTGGTTAATTCCGATAACGAACGAGACCTTGACCTGCT |
| | | AAATAGACGGGTTGACATTTTGTTGGCCCCTTATGTCTTCTTAGAGGG |
| | | ACAATCGACCGTCTAGGTGATGGAGGCAAAAGGCAATAACAGGTCTG |
| | | TGATGCCCTTAGATGTTCCGGGCTGCACGCGCGCTACACTGACAGAG |
| | | ACAACGAGTGGGGCCCCTTGTCCGAAATGACTGGGTAAACTTGTGAA |
| | | ACTTTGTCGTGCTGGGGATGGAGCTTTGTAATTTTTGCTCTTCAACGA |
| | | GGAATTCCTAGTAAGCGCAAGTCATCAGCTTGCGTTGACTACGTCCCT |
| | | GCCCTTTGTACACACCGCCCGTCGCTACTACCGATTGAATGGCTTAGT |
| | | GAGGACTTGGGAGAGTACATCGGGGAGCCAGCAATGGCACCCTGACG |
| | | GCTCAAACTCTTACAAACTTGGTCATTTAGAGGAAGTAAAAGTCGTA |
| | | ACAAGGTATCTGTAGGTGAACCTGCAGATGGATCATTTC |
| 32 | DP32 16S rRNA | ACTGAGCATTGACGTTACTCGCAGAAGAAGCACCGGCTAACTCCGTG |
| | | CCAGCAGCCGCGGTAATACGGAGGGTGCAAGCGTTAATCGGAATTAC |
| | | TGGGCGTAAAGCGCACGCAGGCGGTTTGTTAAGTCAGATGTGAAATC |
| | | CCCGAGCTTAACTTGGGAACTGCATTTGAAACTGGCAAGCTAGAGTC |
| | | TTGTAGAGGGGGGTAGAATTCCAGGTGTAGCGGTGAAATGCGTAGAG |
| | | ATCTGGAGGAATACCGGTGGCGAAGGCGGCCCCCTGGACAAAGACTG |
| | | ACGCTCAGGTGCGAAAGCGTGGGGAGCAAACAGGATTAGATACCCTG |
| | | GTAGTCCACGCTGTAAACGATGTCGACTTGGAGGTTGTGCCCTTGAGG |
| | | CGTGGCTTCCGGAGCTAACGCGTTAAGTCGACCGCCTGGGGAGTACG |
| | | GCCGCAAGGTTAAAACTCAAATGAATTGACGGGGGCCCGCACAAGCG |
| | | GTGGAGCATGTGGTTTAATTCGATGCAACGCGAAGAACCTTACCTACT |
| | | CTTGACATCCAGAGAATTCGCTAGAGATAGCTTAGTGCCTTCGGGAA |
| | | CTCTGAGACAGGTGCTGCATGGCTGTCGTCAGCTCGTGTTGTGAAATG |
| | | TTGGGTTAAGTCCCGCAACGAGCGCAACCCTTATCCTTTGTTGCCAGC |
| | | GAGTAATGTCGGGAACTCAAAGGAGACTGCCGGTGATAAACCGGAG |
| | | GAAGGTGGGGATGACGTCAAGTCATCATGGCCCTTACGAGTAGGGCT |
| | | ACACACGTGCTACAATGGCATATACAAAGAGAAGCGAACTCGCGAGA |
| | | GCAAGCGGACCTCATAAAGTATGTCGTAGTCCGGATTGGAGTCTGCA |
| | | ACTCGACTCCATGAAGTCGGAATCGCTAGTAATCGTAGATCAGAATG |
| | | CTACGGTGAATACGTTCCCGGGCCTTGTACACACCGCCCGTCACACCA |
| | | TGGGAGTGGGTTGCAAAAGAAGTAGGTAGCTTAACCTTCGGGAGGGC |
| | | GCTTACCACTTTGTGATTCATGACTGGGGTGAAGTCGTAACAAGGTAA |
| | | CCGTAGGGGAACCTGCGGTTGGATCACCTCCTT |
| 33 | DP33 16S rRNA | GGAGGAAGGCGTAGAGATCTGGAGGAATACCGGTGGCGAAGGCGGC |
| | | CCCCTGGACAAAGACTGACGCTCAGGTGCGAAAGCGTGGGGAGCAA |
| | | ACAGGATTAGATACCCTGGTAGTCCACGCCGTAAACGATGTCGACTT |
| | | GGAGGTTGTGCCCTTGAGGCGTGGCTTCCGGAGCTAACGCGTTAAGT |
| | | CGACCGCCTGGGGAGTACGGCCGCAAGGTTAAAACTCAAATGAATTG |
| | | ACGGGGGCCCGCACAAGCGGTGGAGCATGTGGTTTAATTCGATGCAA |
| | | CGCGAAGAACCTTACCTGGCCTTGACATCCACGGAATTCGGCAGAGA |
| | | TGCCTTAGTGCCTTCGGGAACCGTGAGACAGGTGCTGCATGGCTGTCG |
| | | TCAGCTCGTGTTGTGAAATGTTGGGTTAAGTCCCGCAACGAGCGCAA |
| | | CCCTTATCCTTTGTTGCCAGCACGTAATGGTGGGAACTCAAAGGAGAC |
| | | TGCCGGTGATAAACCGGAGGAAGGTGGGGATGACGTCAAGTCATCAT |
| | | GGCCCTTACGGCCAGGGCTACACACGTGCTACAATGGCGCATACAAA |
| | | GAGAAGCGACCTCGCGAGAGCAAGCGGACCTCATAAAGTGCGTCGTA |
| | | GTCCGGATCGGAGTCTGCAACTCGACTCCGTGAAGTCGGAATCGCTA |
| | | GTAATCGTAGATCAGAATGCTACGGTGAATACGTTCCCGGGCCTTGTA |
| | | CACACCGCCCGTCACACCATGGGAGTGGGTTGCAAAAGAAGTAGGTA |
| | | GCTTAACCTTCGGGAGGGCGCTTACCACTTTGTGATTCATGACTGGGG |
| | | TGAAGTCGTAACAAGGTAACCGTAGGGGAACCTGCGGTTGGATCACC |
| | | TCCTT |
| 34 | DP34 16S rRNA | TACGGAGAGTTTGATCCTGGCTCAGGACGAACGCTGGCGGCGTGCTT |
| | | AACACATGCAAGTCGAACGATGAAGCCCAGCTTGCTGGGTGGATTAG |
| | | TGGCGAACGGGTGAGTAACACGTGAGTAACCTGCCCTTGACTCTGGG |
| | | ATAAGCGTTGGAAACGACGTCTAATACCGGATACGAGCTTCCACCGC |
| | | ATGGTGAGTTGCTGGAAAGAATTTTGGTCAAGGATGGACTCGCGGCC |
| | | TATCAGCTTGTTGGTGAGGTAATGGCTCACCAAGGCGACGACGGGTA |
| | | GCCGGCCTGAGAGGGTGACCGGCCACACTGGGACTGAGACACGGCCC |
| | | AGACTCCTACGGGAGGCAGCAGTGGGGAATATTGCACAATGGGCGAA |
| | | AGCCTGATGCAGCAACGCCGCGTGAGGGACGACGGCCTTCGGGTTGT |
| | | AAACCTCTTTTAGCAGGGAAGAAGCGAAAGTGACGGTACCTGCAGAA |
| | | AAAGCACCGGCTAACTACGTGCCAGCAGCCGCGGTAATACGTAGGGT |
| | | GCAAGCGTTGTCCGGAATTATTGGGCGTAAAGAGCTCGTAGGCGGTT |
| | | TGTCGCGTCTGCTGTGAAATCCCGAGGCTCAACCTCGGGTCTGCAGTG |
| | | GGTACGGGCAGACTAGAGTGCGGTAGGGGAGATTGGAATTCCTGGTG |
| | | TAGCGGTGGAATGCGCAGATATCAGGAGGAACACCGATGGCGAAGG |
| | | CAGATCTCTGGGCCGCTACTGACGCTGAGGAGCGAAAGGGTGGGGAG |
| | | CAAACAGGCTTAGATACCCTGGTAGTCCACCCCGTAAACGTTGGGCG |
| | | CTAGATGTGGGGACCATTCCACGGTTTCCGTGTCGTAGCTAACGCATT |
| | | AAGCGCCCCGCCTGGGGAGTACGGCCGCAAGGCTAAAACTCAAAGG |
| | | AATTGACGGGGGCCCGCACAAGCGGCGGAGCATGCGGATTAATTCGA |
| | | TGCAACGCGAAGAACCTTACCAAGGCTTGACATATACGAGAACGGGC |
| | | CAGAAATGGTCAACTCTTTGGACACTCGTAAACAGGTGGTGCATGGT |
| | | TGTCGTCAGCTCGTGTCGTGAGATGTTGGGTTAAGTCCCGCAACGAGC |
| | | GCAACCCTCGTTCTATGTTGCCAGCACGTAATGGTGGGAACTCATGGG |
| | | ATACTGCCGGGGTCAACTCGGAGGAAGGTGGGGACGACGTCAAATCA |
| | | TCATGCCCCTTATGTCTTGGGCTTCACGCATGCTACAATGGCCAGTAC |
| | | AAAGGGCTGCAATACCGTAAGGTGGAGCGAATCCCAAAAAGCTGGTC |
| | | CCAGTTCGGATTGAGGTCTGCAACTCGACCTCATGAAGTCGGAGTCG |
| | | CTAGTAATCGCAGATCAGCAACGCTGCGGTGAATACGTTCCCGGGCC |
| | | TTGTACACACCGCCCGTCAAGTCATGAAAGTCGGTAACACCCGAAGC |
| | | CAGTGGCCTAACCGCAAGGATGGAGCTGTCTAAGGTGGGATCGGTAA |
| | | TTAGGACTAAGTCGTAACAAGGTAGCCGTACCGGAAGGTGCGGCTGG |
| | | ATCACCTCCTTT |
| 35 | DP35 16S rRNA | TTGAAGAGTTTGATCATGGCTCAGATTGAACGCTGGCGGCAGGCCTA |
| | | ACACATGCAAGTCGGACGGTAGCACAGAGAGCTTGCTCTTGGGTGAC |
| | | GAGTGGCGGACGGGTGAGTAATGTCTGGGGATCTGCCCGATAGAGGG |
| | | GGATAACCACTGGAAACGGTGGCTAATACCGCATAACGTCGCAAGAC |
| | | CAAAGAGGGGGACCTTCGGGCCTCTCACTATCGGATGAACCCAGATG |
| | | GGATTAGCTAGTAGGCGGGGTAATGGCCCACCTAGGCGACGATCCCT |
| | | AGCTGGTCTGAGAGGATGACCAGCCACACTGGAACTGAGACACGGTC |
| | | CAGACTCCTACGGGAGGCAGCAGTGGGGAATATTGCACAATGGGCGC |
| | | AAGCCTGATGCAGCCATGCCGCGTGTATGAAGAAGGCCTTCGGGTTG |
| | | TAAAGTACTTTCAGCGGGGAGGAAGGCGATGAGGTTAATAACCGCGT |
| | | CGATTGACGTTACCCGCAGAAGAAGCACCGGCTAACTCCGTGCCAGC |
| | | AGCCGCGGTAATACGGAGGGTGCAAGCGTTAATCGGAATTACTGGGC |
| | | GTAAAGCGCACGCAGGCGGTCTGTTAAGTCAGATGTGAAATCCCCGG |
| | | GCTTAACCTGGGAACTGCATTTGAAACTGGCAGGCTTGAGTCTTGTAG |
| | | AGGGGGGTAGAATTCCAGGTGTAGCGGTGAAATGCGTAGAGATCTGG |
| | | AGGAATACCGGTGGCGAAGGCGGCCCCCTGGACAAAGACTGACGCTC |
| | | AGGTGCGAAAGCGTGGGGAGCAAACAGGATTAGATACCCTGGTAGTC |
| | | CACGCCGTAAACGATGTCGACTTGGAGGTTGTTCCCTTGAGGAGTGG |
| | | CTTCCGGAGCTAACGCGTTAAGTCGACCGCCTGGGGAGTACGGCCGC |
| | | AAGGTTAAAACTCAAATGAATTGACGGGGGCCCGCACAAGCGGTGGA |
| | | GCATGTGGTTTAATTCGATGCAACGCGAAGAACCTTACCTACTCTTGA |
| | | CATCCAGCGAACTTAGCAGAGATGCTTTGGTGCCTTCGGGAACGCTG |
| | | AGACAGGTGCTGCATGGCTGTCGTCAGCTCGTGTTGTGAAATGTTGGG |
| | | TTAAGTCCCGCAACGAGCGCAACCCTTATCCTTTGTTGCCAGCGATTC |
| | | GGTCGGGAACTCAAAGGAGACTGCCGGTGATAAACCGGAGGAAGGT |
| | | GGGGATGACGTCAAGTCATCATGGCCCTTACGAGTAGGGCTACACAC |
| | | GTGCTACAATGGCGCATACAAAGAGAAGCGACCTCGCGAGAGCAAG |
| | | CGGACCTCACAAAGTGCGTCGTAGTCCGGATCGGAGTCTGCAACTCG |
| | | ACTCCGTGAAGTCGGAATCGCTAGTAATCGTGGATCAGAATGCCACG |
| | | GTGAATACGTTCCCGGGCCTTGTACACACCGCCCGTCACACCATGGG |
| | | AGTGGGTTGCAAAAGAAGTAGGTAGCTTAACCTTCGGGAGGGCGCTT |
| | | ACCACTTTGTGATTCATTACTGGGGTGAAGTCGTAACAAGGTAACCGT |
| | | AGGGGAACCTGCGGTTGGATCACCTCCTT |
| 36 | DP36 16S rRNA | TTGAAGAGTTTGATCATGGCTCAGATTGAACGCTGGCGGCAGGCCTA |
| | | ACACATGCAAGTCGGACGGTAGCACAGAGAGCTTGCTCTTGGGTGAC |
| | | GAGTGGCGGACGGGTGAGTAATGTCTGGGGATCTGCCCGATAGAGGG |
| | | GGATAACCACTGGAAACGGTGGCTAATACCGCATAACGTCGCAAGAC |
| | | CAAAGAGGGGGACCTTCGGGCCTCTCACTATCGGATGAACCCAGATG |
| | | GGATTAGCTAGTAGGCGGGGTAATGGCCCACCTAGGCGACGATCCCT |
| | | AGCTGGTCTGAGAGGATGACCAGCCACACTGGAACTGAGACACGGTC |
| | | CAGACTCCTACGGGAGGCAGCAGTGGGGAATATTGCACAATGGGCGC |
| | | AAGCCTGATGCAGCCATGCCGCGTGTATGAAGAAGGCCTTCGGGTTG |
| | | TAAAGTACTTTCAGCGGGGAGGAAGGCGATGCGGTTAATAACCGCGT |
| | | CGATTGACGTTACCCGCAGAAGAAGCACCGGCTAACTCCGTGCCAGC |
| | | AGCCGCGGTAATACGGAGGGTGCAAGCGTTAATCGGAATTACTGGGC |
| | | GTAAAGCGCACGCAGGCGGTCTGTTAAGTCAGATGTGAAATCCCCGG |
| | | GCTTAACCTGGGAACTGCATTTGAAACTGGCAGGCTTGAGTCTTGTAG |
| | | AGGGGGGTAGAATTCCAGGTGTAGCGGTGAAATGCGTAGAGATCTGG |
| | | AGGAATACCGGTGGCGAAGGCGGCCCCCTGGACAAAGACTGACGCTC |
| | | AGGTGCGAAAGCGTGGGGAGCAAACAGGATTAGATACCCTGGTAGTC |
| | | CACGCCGTAAACGATGTCGACTTGGAGGTTGTTCCCTTGAGGAGTGG |
| | | CTTCCGGAGCTAACGCGTTAAGTCGACCGCCTGGGGAGTACGGCCGC |
| | | AAGGTTAAAACTCAAATGAATTGACGGGGGCCCGCACAAGCGGTGGA |
| | | GCATGTGGTTTAATTCGATGCAACGCGAAGAACCTTACCTACTCTTGA |
| | | CATC |
| 37 | DP37 16S rRNA | TGAAGAGTTTGATCATGGCTCAGATTGAACGCTGGCGGCAGGCCTAA |
| | | CACATGCAAGTCGAGCGGTAGAGAGAAGCTTGCTTCTCTTGAGAGCG |
| | | GCGGACGGGTGAGTAATGCCTAGGAATCTGCCTGGTAGTGGGGGATA |
| | | ACGTTCGGAAACGAACGCTAATACCGCATACGTCCTACGGGAGAAAG |
| | | CAGGGGACCTTCGGGCCTTGCGCTATCAGATGAGCCTAGGTCGGATT |
| | | AGCTAGTTGGTGGGGTAATGGCTCACCAAGGCGACGATCCGTAACTG |
| | | GTCTGAGAGGATGATCAGTCACACTGGAACTGAGACACGGTCCAGAC |
| | | TCCTACGGGAGGCAGCAGTGGGGAATATTGGACAATGGGCGAAAGCC |
| | | TGATCCAGCCATGCCGCGTGTGTGAAGAAGGTCTTCGGATTGTAAAG |
| | | CACTTTAAGTTGGGAGGAAGGGCCATTACCTAATACGTGATGGTTTTG |
| | | ACGTTACCGACAGAATAAGCACCGGCTAACTCTGTGCCAGCAGCCGC |
| | | GGTAATACAGAGGGTGCAAGCGTTAATCGGAATTACTGGGCGTAAAG |
| | | CGCGCGTAGGTGGTTTGTTAAGTTGGATGTGAAATCCCCGGGCTCAAC |
| | | CTGGGAACTGCATTCAAAACTGACTGACTAGAGTATGGTAGAGGGTG |
| | | GTGGAATTTCCTGTGTAGCGGTGAAATGCGTAGATATAGGAAGGAAC |
| | | ACCAGTGGCGAAGGCGACCACCTGGACTGATACTGACACTGAGGTGC |
| | | GAAAGCGTGGGGAGCAAACAGGATTAGATACCCTGGTAGTCCACGCC |
| | | GTAAACGATGTCAACTAGCCGTTGGGAGCCTTGAGCTCTTAGTGGCG |
| | | CAGCTAACGCATTAAGTTGACCGCCTGGGGAGTACGGCCGCAAGGTT |
| | | AAAACTCAAATGAATTGACGGGGGCCCGCACAAGCGGTGGAGCATGT |
| | | GGTTTAATTCGAAGCAACGCGAAGAACCTTACCAGGCCTTGACATCC |
| | | AATGAACTTTCTAGAGATAGATTGGTGCCTTCGGGAACATTGAGACA |
| | | GGTGCTGCATGGCTGTCGTCAGCTCGTGTCGTGAGATGTTGGGTTAAG |
| | | TCCCGTAACGAGCGCAACCCTTGTCCTTAGTTACCAGCACGTAATGGT |
| | | GGGCACTCTAAGGAGACTGCCGGTGACAAACCGGAGGAAGGTGGGG |
| | | ATGACGTCAAGTCATCATGGCCCTTACGGCCTGGGCTACACACGTGCT |
| | | ACAATGGTCGGTACAGAGGGTTGCCAAGCCGCGAGGTGGAGCTAATC |
| | | CCATAAAACCGATCGTAGTCCGGATCGCAGTCTGCAACTCGACTGCG |
| | | TGAAGTCGGAATCGCTAGTAATCGCGAATCAGAATGTCGCGGTGAAT |
| | | ACGTTCCCGGGCCTTGTACACACCGCCCGTCACACCATGGGAGTGGG |
| | | TTGCACCAGAAGTAGCTAGTCTAACCTTCGGGGGGACGGTTACCACG |
| | | GTGTGATTCATGACTGGGGTGAAGTCGTAACAAGGTAGCCGTAGGGG |
| | | AACCTGCGGCTGGATCACCTCCTT |
| 38 | DP38 16S rRNA | TACGGAGAGTTTGATCCTGGCTCAGGACGAACGCTGGCGGCGTGCTT |
| | | AACACATGCAAGTCGAGCGGTAAGGCCTTTCGGGGTACACGAGCGGC |
| | | GAACGGGTGAGTAACACGTGGGTGATCTGCCCTGCACTCTGGGATAA |
| | | GCTTGGGAAACTGGGTCTAATACCGGATATGACCACAGCATGCATGT |
| | | GTTGTGGTGGAAAGATTTATCGGTGCAGGATGGGCCCGCGGCCTATC |
| | | AGCTTGTTGGTGGGGTAATGGCCTACCAAGGCGACGACGGGTAGCCG |
| | | ACCTGAGAGGGTGACCGGCCACACTGGGACTGAGACACGGCCCAGAC |
| | | TCCTACGGGAGGCAGCAGTGGGGAATATTGCACAATGGGCGGAAGCC |
| | | TGATGCAGCGACGCCGCGTGAGGGATGAAGGCCTTCGGGTTGTAAAC |
| | | CTCTTTCAGCAGGGACGAAGCGTGAGTGACGGTACCTGCAGAAGAAG |
| | | CACCGGCTAACTACGTGCCAGCAGCCGCGGTAATACGTAGGGTGCGA |
| | | GCGTTGTCCGGAATTACTGGGCGTAAAGAGTTCGTAGGCGGTTTGTCG |
| | | CGTCGTTTGTGAAAACCCGGGGCTCAACTTCGGGCTTGCAGGCGATA |
| | | CGGGCAGACTTGAGTGTTTCAGGGGAGACTGGAATTCCTGGTGTAGC |
| | | GGTGAAATGCGCAGATATCAGGAGGAACACCGGTGGCGAAGGCGGG |
| | | TCTCTGGGAAACAACTGACGCTGAGGAACGAAAGCGTGGGTAGCAAA |
| | | CAGGATTAGATACCCTGGTAGTCCACGCCGTAAACGGTGGGCGCTAG |
| | | GTGTGGGTTCCTTCCACGGGATCTGTGCCGTAGCTAACGCATTAAGCG |
| | | CCCCGCCTGGGGAGTACGGCCGCAAGGCTAAAACTCAAAGGAATTGA |
| | | CGGGGGCCCGCACAAGCGGCGGAGCATGTGGATTAATTCGATGCAAC |
| | | GCGAAGAACCTTACCTGGGTTTGACATACACCGGAAAACCGTAGAGA |
| | | TACGGTCCCCCTTGTGGTCGGTGTACAGGTGGTGCATGGCTGTCGTCA |
| | | GCTCGTGTCGTGAGATGTTGGGTTAAGTCCCGCAACGAGCGCAACCC |
| | | TTGTCTTATGTTGCCAGCACGTAATGGTGGGGACTCGTAAGAGACTGC |
| | | CGGGGTCAACTCGGAGGAAGGTGGGGACGACGTCAAGTCATCATGCC |
| | | CCTTATGTCCAGGGCTTCACACATGCTACAATGGCCAGTACAGAGGG |
| | | CTGCGAGACCGTGAGGTGGAGCGAATCCCTTAAAGCTGGTCTCAGTT |
| | | CGGATCGGGGTCTGCAACTCGACCCCGTGAAGTCGGAGTCGCTAGTA |
| | | ATCGCAGATCAGCAACGCTGCGGTGAATACGTTCCCGGGCCTTGTAC |
| | | ACACCGCCCGTCACGTCATGAAAGTCGGTAACACCCGAAGCCGGTGG |
| | | CCTAACCCCTTACGGGGAGGGAGCCGTCGAAGGTGGGATCGGCGATT |
| | | GGGACGAAGTCGTAACAAGGTAGCCGTACCGGAAGGTGCGGCTGGAT |
| | | CACCTCCTTT |
| 39 | DP39 16S rRNA | CTTGAGAGTTTGATCCTGGCTCAGAACGAACGCTGGCGGCAGGCTTA |
| | | ACACATGCAAGTCGAACGCCCCGCAAGGGGAGTGGCAGACGGGTGA |
| | | GTAACGCGTGGGAATCTACCGTGCCCTGCGGAATAGCTCCGGGAAAC |
| | | TGGAATTAATACCGCATACGCCCTACGGGGGAAAGATTTATCGGGGT |
| | | ATGATGAGCCCGCGTTGGATTAGCTAGTTGGTGGGGTAAAGGCCTAC |
| | | CAAGGCGACGATCCATAGCTGGTCTGAGAGGATGATCAGCCACATTG |
| | | GGACTGAGACACGGCCCAAACTCCTACGGGAGGCAGCAGTGGGGAA |
| | | TATTGGACAATGGGCGCAAGCCTGATCCAGCCATGCCGCGTGAGTGA |
| | | TGAAGGCCTTAGGGTTGTAAAGCTCTTTCACCGGAGAAGATAATGAC |
| | | GGTATCCGGAGAAGAAGCCCCGGCTAACTTCGTGCCAGCAGCCGCGG |
| | | TAATACGAAGGGGGCTAGCGTTGTTCGGAATTACTGGGCGTAAAGCG |
| | | CACGTAGGCGGATATTTAAGTCAGGGGTGAAATCCCAGAGCTCAACT |
| | | CTGGAACTGCCTTTGATACTGGGTATCTTGAGTATGGAAGAGGTAAGT |
| | | GGAATTCCGAGTGTAGAGGTGAAATTCGTAGATATTCGGAGGAACAC |
| | | CAGTGGCGAAGGCGGCTTACTGGTCCATTACTGACGCTGAGGTGCGA |
| | | AAGCGTGGGGAGCAAACAGGATTAGATACCCTGGTAGTCCACGCCGT |
| | | AAACGATGAATGTTAGCCGTCGGGCAGTATACTGTTCGGTGGCGCAG |
| | | CTAACGCATTAAACATTCCGCCTGGGGAGTACGGTCGCAAGATTAAA |
| | | ACTCAAAGGAATTGACGGGGGCCCGCACAAGCGGTGGAGCATGTGGT |
| | | TTAATTCGAAGCAACGCGCAGAACCTTACCAGCTCTTGACATTCGGG |
| | | GTTTGGGCAGTGGAGACATTGTCCTTCAGTTAGGCTGGCCCCAGAAC |
| | | AGGTGCTGCATGGCTGTCGTCAGCTCGTGTCGTGAGATGTTGGGTTAA |
| | | GTCCCGCAACGAGCGCAACCCTCGCCCTTAGTTGCCAGCATTTAGTTG |
| | | GGCACTCTAAGGGGACTGCCGGTGATAAGCCGAGAGGAAGGTGGGG |
| | | ATGACGTCAAGTCCTCATGGCCCTTACGGGCTGGGCTACACACGTGCT |
| | | ACAATGGTGGTGACAGTGGGCAGCGAGACAGCGATGTCGAGCTAATC |
| | | TCCAAAAGCCATCTCAGTTCGGATTGCACTCTGCAACTCGAGTGCATG |
| | | AAGTTGGAATCGCTAGTAATCGCAGATCAGCATGCTGCGGTGAATAC |
| | | GTTCCCGGGCCTTGTACACACCGCCCGTCACACCATGGGAGTTGGTTT |
| | | TACCCGAAGGTAGTGCGCTAACCGCAAGGAGGCAGCTAACCACGGTA |
| | | GGGTCAGCGACTGGGGTGAAGTCGTAACAAGGTAGCCGTAGGGGAA |
| | | CCTGCGGCTGGATCACCTCCTTT |
| 40 | DP40 16S rRNA | TTGACGTTACCCGCAGAAGAAGCACCGGCTAACTCCGTGCCAGCAGC |
| | | CGCGGTAATACGGAGGGTGCAAGCGTTAATCGGAATTACTGGGCGTA |
| | | AAGCGCACGCAGGCGGTCTGTTAAGTCAGATGTGAAATCCCCGGGCT |
| | | TAACCTGGGAACTGCATTTGAAACTGGCAGGCTTGAGTCTTGTAGAG |
| | | GGGGGTAGAATTCCAGGTGTAGCGGTGAAATGCGTAGAGATCTGGAG |
| | | GAATACCGGTGGCGAAGGCGGCCCCCTGGACAAAGACTGACGCTCAG |
| | | GTGCGAAAGCGTGGGGAGCAAACAGGATTAGATACCCTGGTAGTCCA |
| | | CGCCGTAAACGATGTCGACTTGGAGGTTGTTCCCTTGAGGAGTGGCTT |
| | | CCGGAGCTAACGCGTTAAGTCGACCGCCTGGGGAGTACGGCCGCAAG |
| | | GTTAAAACTCAAATGAATTGACGGGGGCCCGCACAAGCGGTGGAGCA |
| | | TGTGGTTTAATTCGATGCAACGCGAAGAACCTTACCTACTCTTGACAT |
| | | CCAGAGAACTTTCCAGAGATGGATTGGTGCCTTCGGGAACTCTGAGA |
| | | CAGGTGCTGCATGGCTGTCGTCAGCTCGTGTTGTGAAATGTTGGGTTA |
| | | AGTCCCGCAACGAGCGCAACCCTTATCCTTTGTTGCCAGCGCGTGATG |
| | | GCGGGAACTCAAAGGAGACTGCCGGTGATAAACCGGAGGAAGGTGG |
| | | GGATGACGTCAAGTCATCATGGCCCTTACGAGTAGGGCTACACACGT |
| | | GCTACAATGGCGCATACAAAGAGAAGCGACCTCGCGAGAGCAAGCG |
| | | GACCTCACAAAGTGCGTCGTAGTCCGGATCGGAGTCTGCAACTCGAC |
| | | TCCGTGAAGTCGGAATCGCTAGTAATCGTGGATCAGAATGCCACGGT |
| | | GAATACGT |
| 41 | DP41 16S rRNA | GTGGAGAGTTTGATCCTGGCTCAGGATGAACGCTGGCGGCGTGCTTA |
| | | ACACATGCAAGTCGAACGGAAAGGCCCAAGCTTGCTTGGGTACTCGA |
| | | GTGGCGAACGGGTGAGTAACACGTGGGTGATCTGCCCTGCACTTCGG |
| | | GATAAGCCTGGGAAACTGGGTCTAATACCGGATAGGACGATGGTTTG |
| | | GATGCCATTGTGGAAAGTTTTTTCGGTGTGGGATGAGCTCGCGGCCTA |
| | | TCAGCTTGTTGGTGGGGTAATGGCCTACCAAGGCGTCGACGGGTAGC |
| | | CGGCCTGAGAGGGTGTACGGCCACATTGGGACTGAGATACGGCCCAG |
| | | ACTCCTACGGGAGGCAGCAGTGGGGAATATTGCACAATGGGCGCAAG |
| | | CCTGATGCAGCGACGCCGCGTGGGGGATGACGGCCTTCGGGTTGTAA |
| | | ACTCCTTTCGCTAGGGACGAAGCGTTTTGTGACGGTACCTGGAGAAG |
| | | AAGCACCGGCTAACTACGTGCCAGCAGCCGCGGTAATACGTAGGGTG |
| | | CGAGCGTTGTCCGGAATTACTGGGCGTAAAGAGCTCGTAGGTGGTTT |
| | | GTCGCGTCGTTTGTGTAAGCCCGCAGCTTAACTGCGGGACTGCAGGC |
| | | GATACGGGCATAACTTGAGTGCTGTAGGGGAGACTGGAATTCCTGGT |
| | | GTAGCGGTGGAATGCGCAGATATCAGGAGGAACACCGATGGCGAAG |
| | | GCAGGTCTCTGGGCAGTAACTGACGCTGAGGAGCGAAAGCATGGGTA |
| | | GCGAACAGGATTAGATACCCTGGTAGTCCATGCCGTAAACGGTGGGC |
| | | GCTAGGTGTGAGTCCCTTCCACGGGGTTCGTGCCGTAGCTAACGCATT |
| | | AAGCGCCCCGCCTGGGGAGTACGGCCGCAAGGCTAAAACTCAAAGG |
| | | AATTGACGGGGGCCCGCACAAGCGGCGGAGCATGTGGATTAATTCGA |
| | | TGCAACGCGAAGAACCTTACCTGGGCTTGACATACACCAGATCGCCG |
| | | TAGAGATACGGTTTCCCTTTGTGGTTGGTGTACAGGTGGTGCATGGTT |
| | | GTCGTCAGCTCGTGTCGTGAGATGTTGGGTTAAGTCCCGCAACGAGC |
| | | GCAACCCTTGTCTTATGTTGCCAGCACGTGATGGTGGGGACTCGTGAG |
| | | AGACTGCCGGGGTTAACTCGGAGGAAGGTGGGGATGACGTCAAATCA |
| | | TCATGCCCCTTATGTCCAGGGCTTCACACATGCTACAATGGTCGGTAC |
| | | AACGCGCATGCGAGCCTGTGAGGGTGAGCGAATCGCTGTGAAAGCCG |
| | | GTCGTAGTTCGGATTGGGGTCTGCAACTCGACCCCATGAAGTCGGAG |
| | | TCGCTAGTAATCGCAGATCAGCAACGCTGCGGTGAATACGTTCCCGG |
| | | GCCTTGTACACACCGCCCGTCACACCATGGGAGTGGGTTGCAAAAGA |
| | | AGTAGGTAGCTTAACCTTCGGGAGGGCGCTTACCACTTTGTGAT |
| 42 | DP42 16S rRNA | TGAAGAGTTTGATCATGGCTCAGATTGAACGCTGGCGGCAGGCCTAA |
| | | CACATGCAAGTCGAGCGGTAGAGAGGTGCTTGCACCTCTTGAGAGCG |
| | | GCGGACGGGTGAGTAATACCTAGGAATCTGCCTGATAGTGGGGGATA |
| | | ACGTTCGGAAACGGACGCTAATACCGCATACGTCCTACGGGAGAAAG |
| | | CAGGGGACCTTCGGGCCTTGCGCTATCAGATGAGCCTAGGTCGGATT |
| | | AGCTAGTTGGTGAGGTAATGGCTCACCAAGGCTACGATCCGTAACTG |
| | | GTCTGAGAGGATGATCAGTCACACTGGAACTGAGACACGGTCCAGAC |
| | | TCCTACGGGAGGCAGCAGTGGGGAATATTGGACAATGGGCGAAAGCC |
| | | TGATCCAGCCATGCCGCGTGTGTGAAGAAGGTCTTCGGATTGTAAAG |
| | | CACTTTAAGTTGGGAGGAAGGGCATTAACCTAATACGTTAGTGTCTTG |
| | | ACGTTACCGACAGAATAAGCACCGGCTAACTCTGTGCCAGCAGCCGC |
| | | GGTAATACAGAGGGTGCAAGCGTTAATCGGAATTACTGGGCGTAAAG |
| | | CGCGCGTAGGTGGTTTGTTAAGTTGAATGTGAAATCCCCGGGCTCAAC |
| | | CTGGGAACTGCATCCAAAACTGGCAAGCTAGAGTATGGTAGAGGGTA |
| | | GTGGAATTTCCTGTGTAGCGGTGAAATGCGTAGATATAGGAAGGAAC |
| | | ACCAGTGGCGAAGGCGACTACCTGGACTGATACTGACACTGAGGTGC |
| | | GAAAGCGTGGGGAGCAAACAGGATTAGATACCCTGGTAGTCCACGCC |
| | | GTAAACGATGTCAACTAGCCGTTGGGAACCTTGAGTTCTTAGTGGCGC |
| | | AGCTAACGCATTAAGTTGACCGCCTGGGGAGTACGGCCGCAAGGTTA |
| | | AAACTCAAATGAATTGACGGGGGCCCGCACAAGCGGTGGAGCATGTG |
| | | GTTTAATTCGAAGCAACGCGAAGAACCTTACCAGGCCTTGACATCCA |
| | | ATGAACTTTCCAGAGATGGATTGGTGCCTTCGGGAACATTGAGACAG |
| | | GTGCTGCATGGCTGTCGTCAGCTCGTGTCGTGAGATGTTGGGTTAAGT |
| | | CCCGTAACGAGCGCAACCCTTGTCCTTAGTTACCAGCACGTAATGGTG |
| | | GGCACTCTAAGGAGACTGCCGGTGACAAACCGGAGGAAGGTGGGGA |
| | | TGACGTCAAGTCATCATGGCCCTTACGGCCTGGGCTACACACGTGCTA |
| | | CAATGGTCGGTACAAAGGGTTGCCAAGCCGCGAGGTGGAGCTAATCC |
| | | CATAAAACCGATCGTAGTCCGGATCGCAGTCTGCAACTCGACTGCGT |
| | | GAAGTCGGAATCGCTAGTAATCGTGAATCAGAATGTCACGGTGAATA |
| | | CGTTCCCGGGCCTTGTACACACCGCCCGTCACACCATGGGAGTGGGTT |
| | | GCACCAGAAGTAGCTAGTCTAACCCTCGGGAGGACGGTTACCACGGT |
| | | GTGATTCATGACTGGGGTGAAGTCGTAACAAGGTAGCCGTAGGGGAA |
| | | CCTGCGGCTGGATCACCTCCTT |
| 43 | DP43 16S rRNA | CTGAGTTTGATCCTGGCTCAGATTGAACGCTGGCGGCATGCCTTACAC |
| | | ATGCAAGTCGAACGGCAGCACGGAGCTTGCTCTGGTGGCGAGTGGCG |
| | | AACGGGTGAGTAATATATCGGAACGTACCCTGGAGTGGGGGATAACG |
| | | TAGCGAAAGTTACGCTAATACCGCATACGATCTAAGGATGAAAGTGG |
| | | GGGATCGCAAGACCTCATGCTCGTGGAGCGGCCGATATCTGATTAGC |
| | | TAGTTGGTAGGGTAAAAGCCTACCAAGGCATCGATCAGTAGCTGGTC |
| | | TGAGAGGACGACCAGCCACACTGGAACTGAGACACGGTCCAGACTCC |
| | | TACGGGAGGCAGCAGTGGGGAATTTTGGACAATGGGCGAAAGCCTGA |
| | | TCCAGCAATGCCGCGTGAGTGAAGAAGGCCTTCGGGTTGTAAAGCTC |
| | | TTTTGTCAGGGAAGAAACGGTGAGAGCTAATATCTCTTGCTAATGAC |
| | | GGTACCTGAAGAATAAGCACCGGCTAACTACGTGCCAGCAGCCGCGG |
| | | TAATACGTAGGGTGCAAGCGTTAATCGGAATTACTGGGCGTAAAGCG |
| | | TGCGCAGGCGGTTTTGTAAGTCTGATGTGAAATCCCCGGGCTCAACCT |
| | | GGGAATTGCATTGGAGACTGCAAGGCTAGAATCTGGCAGAGGGGGGT |
| | | AGAATTCCACGTGTAGCAGTGAAATGCGTAGATATGTGGAGGAACAC |
| | | CGATGGCGAAGGCAGCCCCCTGGGTCAAGATTGACGCTCATGCACGA |
| | | AAGCGTGGGGAGCAAACAGGATTAGATACCCTGGTAGTCCACGCCCT |
| | | AAACGATGTCTACTAGTTGTCGGGTCTTAATTGACTTGGTAACGCAGC |
| | | TAACGCGTGAAGTAGACCGCCTGGGGAGTACGGTCGCAAGATTAAAA |
| | | CTCAAAGGAATTGACGGGGACCCGCACAAGCGGTGGATGATGTGGAT |
| | | TAATTCGATGCAACGCGAAAAACCTTACCTACCCTTGACATGGCTGG |
| | | AATCCTTGAGAGATCAGGGAGTGCTCGAAAGAGAACCAGTACACAGG |
| | | TGCTGCATGGCTGTCGTCAGCTCGTGTCGTGAGATGTTGGGTTAAGTC |
| | | CCGCAACGAGCGCAACCCTTGTCATTAGTTGCTACGAAAGGGCACTC |
| | | TAATGAGACTGCCGGTGACAAACCGGAGGAAGGTGGGGATGACGTC |
| | | AAGTCCTCATGGCCCTTATGGGTAGGGCTTCACACGTCATACAATGGT |
| | | ACATACAGAGCGCCGCCAACCCGCGAGGGGGAGCTAATCGCAGAAA |
| | | GTGTATCGTAGTCCGGATTGTAGTCTGCAACTCGACTGCATGAAGTTG |
| | | GAATCGCTAGTAATCGCGGATCAGCATGTCGCGGTGAATACGTTCCC |
| | | GGGTCTTGTACACACCGCCCGTCACACCATGGGAGCGGGTTTTACCA |
| | | GAAGTAGGTAGCTTAACCGTAAGGAGGGCGCTTACCACGGTAGGATT |
| | | CGTGACTGGGGTGAAGTCGTAACAAGGTAGCCGTATCGGAAGGTGCG |
| | | GCTGGATCACCTCCTTT |
| 44 | DP44 16S rRNA | TGGCGGCATGCCTTACACATGCAAGTCGAACGGCAGCATAGGAGCTT |
| | | GCTCCTGATGGCGAGTGGCGAACGGGTGAGTAATATATCGGAACGTG |
| | | CCCTAGAGTGGGGGATAACTAGTCGAAAGACTAGCTAATACCGCATA |
| | | CGATCTACGGATGAAAGTGGGGGATCGCAAGACCTCATGCTCCTGGA |
| | | GCGGCCGATATCTGATTAGCTAGTTGGTGGGGTAAAAGCTCACCAAG |
| | | GCGACGATCAGTAGCTGGTCTGAGAGGACGACCAGCCACACTGGGAC |
| | | TGAGACACGGCCCAGACTCCTACGGGAGGCAGCAGTGGGGAATTTTG |
| | | GACAATGGGGGCAACCCTGATCCAGCAATGCCGCGTGAGTGAAGAAG |
| | | GCCTTCGGGTTGTAAAGCTCTTTTGTCAGGGAAGAAACGGTTCTGGAT |
| | | AATACCTAGGACTAATGACGGTACCTGAAGAATAAGCACCGGCTAAC |
| | | TACGTGCCAGCAGCCGCGGTAATACGTAGGGTGCAAGCGTTAATCGG |
| | | AATTACTGGGCGTAAAGCGTGCGCAGGCGGTTGTGTAAGTCAGATGT |
| | | GAAATCCCCGGGCTCAACCTGGGAATTGCATTTGAGACTGCACGGCT |
| | | AGAGTGTGTCAGAGGGGGGTAGAATTCCACGTGTAGCAGTGAAATGC |
| | | GTAGATATGTGGAGGAATACCGATGGCGAAGGCAGCCCCCTGGGATA |
| | | ACACTGACGCTCATGCACGAAAGCGTGGGGAGCAAACAGGATTAGAT |
| | | ACCCTGGTAGTCCACGCCCTAAACGATGTCTACTAGTTGTCGGGTCTT |
| | | AATTGACTTGGTAACGCAGCTAACGCGTGAAGTAGACCGCCTGGGGA |
| | | GTACGGTCGCAAGATTAAAACTCAAAGGAATTGACGGGGACCCGCAC |
| | | AAGCGGTGGATGATGTGGATTAATTCGATGCAACGCGAAAAACCTTA |
| | | CCTACCCTTGACATGGATGGAATCCCGAAGAGATTTGGGAGTGCTCG |
| | | AAAGAGAACCATCACACAGGTGCTGCATGGCTGTCGTCAGCTCGTGT |
| | | CGTGAGATGTTGGGTTAAGTCCCGCAACGAGCGCAACCCTTGTCATTA |
| | | GTTGCTACGAAAGGGCACTCTAATGAGACTGCCGGTGACAAACCGGA |
| | | GGAAGGTGGGGATGACGTCAAGTCCTCATGGCCCTTATGGGTAGGGC |
| | | TTCACACGTCATACAATGGTACATACAGAGGGCCGCCAACCCGCGAG |
| | | GGGGAGCTAATCCCAGAAAGTGTATCGTAGTCCGGATTGGAGTCTGC |
| | | AACTCGACTCCATGAAGTTGGAATCGCTAGTAATCGCGGATCAGCAT |
| | | GTCGCGGTGAATACGTTCCCGGGTCTTGTACACACCGCCCGTCACACC |
| | | ATGGGAGCGGGTTTTACCAGAAGTGGGTAGCCTAACCGCAAGGAGGG |
| | | CGCTCACCACGGTAGGATTCGTGACTGGGGTGAAGTCGTAACAAGGT |
| | | AGCCGTATCGGAAGGTGCGGCTGGATCACCTCCTTT |
| 45 | DP45 16S rRNA | TACGGAGAGTTTGATCCTGGCTCAGGACGAACGCTGGCGGCGTGCTT |
| | | AACACATGCAAGTCGAACGGTGACGCTAGAGCTTGCTCTGGTTGATC |
| | | AGTGGCGAACGGGTGAGTAACACGTGAGTAACCTGCCCTTGACTCTG |
| | | GGATAACTCCGGGAAACCGGGGCTAATACCGGATACGAGACGCGACC |
| | | GCATGGTCGGCGTCTGGAAAGTTTTTCGGTCAAGGATGGACTCGCGG |
| | | CCTATCAGCTTGTTGGTGAGGTAATGGCTCACCAAGGCGTCGACGGG |
| | | TAGCCGGCCTGAGAGGGCGACCGGCCACACTGGGACTGAGACACGGC |
| | | CCAGACTCCTACGGGAGGCAGCAGTGGGGAATATTGCACAATGGGCG |
| | | AAAGCCTGATGCAGCGACGCCGCGTGAGGGATGAAGGCCTTCGGGTT |
| | | GTAAACCTCTTTCAGTAGGGAAGAAGCGAAAGTGACGGTACCTGCAG |
| | | AAGAAGCGCCGGCTAACTACGTGCCAGCAGCCGCGGTAATACGTAGG |
| | | GCGCAAGCGTTGTCCGGAATTATTGGGCGTAAAGAGCTCGTAGGCGG |
| | | TTTGTCGCGTCTGGTGTGAAAACTCAAGGCTCAACCTTGAGCTTGCAT |
| | | CGGGTACGGGCAGACTAGAGTGTGGTAGGGGTGACTGGAATTCCTGG |
| | | TGTAGCGGTGGAATGCGCAGATATCAGGAGGAACACCGATGGCGAA |
| | | GGCAGGTCACTGGGCCACTACTGACGCTGAGGAGCGAAAGCATGGGG |
| | | AGCGAACAGGATTAGATACCCTGGTAGTCCATGCCGTAAACGTTGGG |
| | | CACTAGGTGTGGGGCTCATTCCACGAGTTCCGCGCCGCAGCTAACGC |
| | | ATTAAGTGCCCCGCCTGGGGAGTACGGCCGCAAGGCTAAAACTCAAA |
| | | GGAATTGACGGGGGCCCGCACAAGCGGCGGAGCATGCGGATTAATTC |
| | | GATGCAACGCGAAGAACCTTACCAAGGCTTGACATACACCGGAATCA |
| | | TGCAGAGATGTGTGCGTCTTCGGACTGGTGTACAGGTGGTGCATGGTT |
| | | GTCGTCAGCTCGTGTCGTGAGATGTTGGGTTAAGTCCCGCAACGAGC |
| | | GCAACCCTCGTCCTATGTTGCCAGCACGTTATGGTGGGGACTCATAGG |
| | | AGACTGCCGGGGTCAACTCGGAGGAAGGTGGGGATGACGTCAAATCA |
| | | TCATGCCCCTTATGTCTTGGGCTTCACGCATGCTACAATGGCCGGTAC |
| | | AAAGGGCTGCGATACCGCGAGGTGGAGCGAATCCCAAAAAGCCGGT |
| | | CTCAGTTCGGATTGGGGTCTGCAACTCGACCCCATGAAGTCGGAGTC |
| | | GCTAGTAATCGCAGATCAGCAACGCTGCGGTGAATACGTTCCCGGGC |
| | | CTTGTACACACCGCCCGTCAAGTCACGAAAGTCGGTAACACCCGAAG |
| | | CCGGTGGCCTAACCCCTTGTGGGATGGAGCCGTCGAAGGTGGGATTG |
| | | GCGATTGGGACTAAGTCGTAACAAGGTAGCCGTACCGGAAGGTGCGG |
| | | CTGGATCACCTCCTTT |
| 46 | DP46 16S rRNA | TTGAAGAGTTTGATCATGGCTCAGATTGAACGCTGGCGGCAGGCCTA |
| | | ACACATGCAAGTCGGACGGTAGCACAGAGGAGCTTGCTCCTTGGGTG |
| | | ACGAGTGGCGGACGGGTGAGTAATGTCTGGGGATCTGCCCGATAGAG |
| | | GGGGATAACCACTGGAAACGGTGGCTAATACCGCATAACGTCGCAAG |
| | | ACCAAAGAGGGGGACCTTCGGGCCTCTCACTATCGGATGAACCCAGA |
| | | TGGGATTAGCTAGTAGGCGGGGTAATGGCCCACCTAGGCGACGATCC |
| | | CTAGCTGGTCTGAGAGGATGACCAGCCACACTGGAACTGAGACACGG |
| | | TCCAGACTCCTACGGGAGGCAGCAGTGGGGAATATTGCACAATGGGC |
| | | GCAAGCCTGATGCAGCCATGCCGCGTGTATGAAGAAGGCCTTCGGGT |
| | | TGTAAAGTACTTTCAGCGGGGAGGAAGGCGACAGGGTTAATAACCCT |
| | | GTCGATTGACGTTACCCGCAGAAGAAGCACCGGCTAACTCCGTGCCA |
| | | GCAGCCGCGGTAATACGGAGGGTGCAAGCGTTAATCGGAATTACTGG |
| | | GCGTAAAGCGCACGCAGGCGGTCTGTTAAGTCAGATGTGAAATCCCC |
| | | GGGCTTAACCTGGGAACTGCATTTGAAACTGGCAGGCTTTAGTCTTGT |
| | | AGAGTGGGGTAGAATTCCAGGTGTAGCGGTGAAATGCGTAGAGATGT |
| | | GGAGGAACACCAGTGGCGAAGGCGGCTTTTTGGTCTGTAACTGACGC |
| | | TGAGGCGCGAAAGCGTGGGGAGCAAACAGGATTAGATACCCTGGTA |
| | | GTCCACGCCGTAAACGATGAGTGCTAAGTGTT |
| 47 | DP47 16S rRNA | AGGGTGCAAGCGTTAATCGGAATTACTGGGCGTAAAGCGCGCGTAGG |
| | | TGGTTTGTTAAGTTGAATGTGAAATCCCCGGGCTCAACCTGGGAACTG |
| | | CATTTGAAACTGGCAAGCTAGAGTCTCGTAGAGGGGGGTAGAATTCC |
| | | AGGTGTAGCGGTGAAATGCGTAGAGATCTGGAGGAATACCGGTGGCG |
| | | AAGGCGGCCCCCTGGACGAAGACTGACGCTCAGGTGCGAAAGCGTGG |
| | | GGAGCAAACAGGATTAGATACCCTGGTAGTCCACGCCGTAAACGATG |
| | | TCAACTAGCCGTTGGAAGCCTTGAGCTTTTAGTGGCGCAGCTAACGCA |
| | | TTAAGTTGACCGCCTGGGGAGTACGGCCGCAAGGTTAAAACTCAAAT |
| | | GAATTGACGGGGGCCCGCACAAGCGGTGGAGCATGTGGTTTAATTCG |
| | | AAGCAACGCGAAGAACCTTACCAGGCCTTGACATCCAATGAACTTTC |
| | | TAGAGATAGATTGGTGCCTTCGGGAACATTGAGACAGGTGCTGCATG |
| | | GCTGTCGTCAGCTCGTGTCGTGAGATGTTGGGTTAAGTCCCGCAACGA |
| | | GCGCAACCCTTGTCCTGTGTTGCCAGCGCGTAATGGCGGGGACTCGC |
| | | AGGAGACTGCCGGGGTCAACTCGGAGGAAGGTGGGGATGACGTCAA |
| | | ATCATCATGCCCCTTATGTCTTGGGCTTCACGCATGCTACAATGGCCG |
| | | GTACAAAGGGCTGCAATACCGTGAGGTGGAGCGAATCCCAAAAAGCC |
| | | GGTCCCAGTTCGGATTGAGGTCTGCAACTCGACCTCATGAAGTCGGA |
| | | GTCGCTAGTAATCGCAGATCAGCAACGCTGCGGTGAATACGTTCCCG |
| | | GGTCTTGTACACACCGCCCGTCAAGTCATGAAAGTCGGTAACACCTG |
| | | AAGCCGGTGGCCCAACCCTTGTGGAGGGAGCCGTCGAAGGTGGGATC |
| | | GGTAATTAGGACTAAGT |
| 48 | DP48 16S rRNA | CATGGAGAGTTTGATCCTGGCTCAGGACGAACGCTGGCGGCGTGCCT |
| | | AATACATGCAAGTCGAGCGGACAGATGGGAGCTTGCTCCCTGATGTT |
| | | AGCGGCGGACGGGTGAGTAACACGTGGGTAACCTGCCTGTAAGACTG |
| | | GGATAACTCCGGGAAACCGGGGCTAATACCGGATGCTTGATTGAACC |
| | | GCATGGTTCAATTATAAAAGGTGGCTTTTAGCTACCACTTACAGATGG |
| | | ACCCGCGGCGCATTAGCTAGTTGGTGAGGTAACGGCTCACCAAGGCA |
| | | ACGATGCGTAGCCGACCTGAGAGGGTGATCGGCCACACTGGGACTGA |
| | | GACACGGCCCAGACTCCTACGGGAGGCAGCAGTAGGGAATCTTCCGC |
| | | AATGGACGAAAGTCTGACGGAGCAACGCCGCGTGAGTGATGAAGGTT |
| | | TTCGGATCGTAAAACTCTGTTGTTAGGGAAGAACAAGTACCGTTCGA |
| | | ATAGGGCGGTACCTTGACGGTACCTAACCAGAAAGCCACGGCTAACT |
| | | ACGTGCCAGCAGCCGCGGTAATACGTAGGTGGCAAGCGTTGTCCGGA |
| | | ATTATTGGGCGTAAAGCGCGCGCAGGCGGTTTCTTAAGTCTGATGTGA |
| | | AAGCCCCCGGCTCAACCGGGGAGGGTCATTGGAAACTGGGGAACTTG |
| | | AGTGCAGAAGAGGAGAGTGGAATTCCACGTGTAGCGGTGAAATGCGT |
| | | AGAGATGTGGAGGAACACCAGTGGCGAAGGCGACTCTCTGGTCTGTA |
| | | ACTGACGCTGAGGCGCGAAAGCGTGGGGAGCGAACAGGATTAGATA |
| | | CCCTGGTAGTCCACGCCGTAAACGATGAGTGCTAAGTGTTAGAGGGT |
| | | TTCCGCCCTTTAGTGCTGCAGCAAACGCATTAAGCACTCCGCCTGGGG |
| | | AGTACGGTCGCAAGACTGAAACTCAAAGGAATTGACGGGGGCCCGCA |
| | | CAAGCGGTGGAGCATGTGGTTTAATTCGAAGCAACGCGAAGAACCTT |
| | | ACCAGGTCTTGACATCCTCTGACAACCCTAGAGATAGGGCTTCCCCTT |
| | | CGGGGGCAGAGTGACAGGTGGTGCATGGTTGTCGTCAGCTCGTGTCG |
| | | TGAGATGTTGGGTTAAGTCCCGCAACGAGCGCAACCCTTGATCTTAGT |
| | | TGCCAGCATTCAGTTGGGCACTCTAAGGTGACTGCCGGTGACAAACC |
| | | GGAGGAAGGTGGGGATGACGTCAAATCATCATGCCCCTTATGACCTG |
| | | GGCTACACACGTGCTACAATGGGCAGAACAAAGGGCAGCGAAGCCG |
| | | CGAGGCTAAGCCAATCCCACAAATCTGTTCTCAGTTCGGATCGCAGTC |
| | | TGCAACTCGACTGCGTGAAGCTGGAATCGCTAGTAATCGCGGATCAG |
| | | CATGCCGCGGTGAATACGTTCCCGGGCCTTGTACACACCGCCCGTCAC |
| | | ACCACGAGAGTTTGTAACACCCGAAGTCGGTGAGGTAACCTTTTGGA |
| | | GCCAGCCGCCGAAGGTGGGACAGATGATTGGGGTGAAGTCGTAACAA |
| | | GGTAGCCGTATCGGAAGGTGCGGCTGGATCACCTCCTTT |
| 49 | DP49 16S rRNA | TATGGAGAGTTTGATCCTGGCTCAGGACGAACGCTGGCGGCGTGCCT |
| | | AATACATGCAAGTCGAGCGGACGTTTTTGAAGCTTGCTTCAAAAACG |
| | | TTAGCGGCGGACGGGTGAGTAACACGTGGGCAACCTGCCTTATCGAC |
| | | TGGGATAACTCCGGGAAACCGGGGCTAATACCGGATAATATCTAGCA |
| | | CCTCCTGGTGCAAGATTAAAAGAGGGCCTTCGGGCTCTCACGGTGAG |
| | | ATGGGCCCGCGGCGCATTAGCTAGTTGGAGAGGTAATGGCTCCCCAA |
| | | GGCGACGATGCGTAGCCGACCTGAGAGGGTGATCGGCCACACTGGGA |
| | | CTGAGACACGGCCCAGACTCCTACGGGAGGCAGCAGTAGGGAATCTT |
| | | CCGCAATGGACGAAAGTCTGACGGAGCAACGCCGCGTGAGTGATGAA |
| | | GGGTTTCGGCTCGTAAAGCTCTGTTATGAGGGAAGAACACGTACCGT |
| | | TCGAATAGGGCGGTACCTTGACGGTACCTCATCAGAAAGCCACGGCT |
| | | AACTACGTGCCAGCAGCCGCGGTAATACGTAGGTGGCAAGCGTTGTC |
| | | CGGAATTATTGGGCGTAAAGCGCGCGCAGGCGGCCTTTTAAGTCTGA |
| | | TGTGAAATCTTGCGGCTCAACCGCAAGCGGTCATTGGAAACTGGGAG |
| | | GCTTGAGTACAGAAGAGGAGAGTGGAATTCCACGTGTAGCGGTGAAA |
| | | TGCGTAGATATGTGGAGGAACACCAGTGGCGAAGGCGACTCTCTGGT |
| | | CTGTAACTGACGCTGAGGCGCGAAAGCGTGGGGAGCAAACAGGATTA |
| | | GATACCCTGGTAGTCCACGCCGTAAACGATGAGTGCTAGGTGTTAGG |
| | | GGTTTCGATGCCCGTAGTGCCGAAGTTAACACATTAAGCACTCCGCCT |
| | | GGGGAGTACGGCCGCAAGGCTGAAACTCAAAGGAATTGACGGGGGC |
| | | CCGCACAAGCAGTGGAGCATGTGGTTTAATTCGAAGCAACGCGAAGA |
| | | ACCTTACCAGGTCTTGACATCCTTTGACCACTCTGGAGACAGAGCTTC |
| | | CCCTTCGGGGGCAAAGTGACAGGTGGTGCATGGTTGTCGTCAGCTCG |
| | | TGTCGTGAGATGTTGGGTTAAGTCCCGCAACGAGCGCAACCCTTGAC |
| | | CTTAGTTGCCAGCATTTAGTTGGGCACTCTAAGGTGACTGCCGGTGAC |
| | | AAACCGGAGGAAGGTGGGGATGACGTCAAATCATCATGCCCCTTATG |
| | | ACCTGGGCTACACACGTGCTACAATGGATGGTACAAAGGGTTGCGAA |
| | | GCCGCGAGGTGAAGCCAATCCCATAAAGCCATTCTCAGTTCGGATTG |
| | | TAGGCTGCAACTCGCCTGCATGAAGCTGGAATTGCTAGTAATCGCGG |
| | | ATCAGCATGCCGCGGTGAATACGTTCCCGGGCCTTGTACACACCGCCC |
| | | GTCACACCACGAGAGTTTGTAACACCCGAAGTCGGTGAGGTAACCTT |
| | | TTGGAGCCAGCCGCCGAAGGTGGGACAGATGATTGGGGTGAAGTCGT |
| | | AACAAGGTAGCCGTATCGGAAGGTGCGGCTGGATCACCTCCTTT |
| 50 | DP50 16S rRNA | TTGAAGAGTTTGATCATGGCTCAGATTGAACGCTGGCGGCAGGCCTA |
| | | ACACATGCAAGTCGAACGGTAGCACAGAGAGCTTGCTCTTGGGTGAC |
| | | GAGTGGCGGACGGGTGAGTAATGTCTGGGAAACTGCCCGATGGAGGG |
| | | GGATAACTACTGGAAACGGTAGCTAATACCGCATAACGTCGCAAGAC |
| | | CAAAGTGGGGGACCTTCGGGCCTCACACCATCGGATGTGCCCAGATG |
| | | GGATTAGCTAGTAGGTGGGGTAATGGCTCACCTAGGCGACGATCCCT |
| | | AGCTGGTCTGAGAGGATGACCAGCCACACTGGAACTGAGACACGGTC |
| | | CAGACTCCTACGGGAGGCAGCAGTGGGGAATATTGCACAATGGGCGC |
| | | AAGCCTGATGCAGCCATGCCGCGTGTATGAAGAAGGCCTTCGGGTTG |
| | | TAAAGTACTTTCAGCGAGGAGGAAGGCATTGTGGTTAATAACCGCAG |
| | | TGATTGACGTTACTCGCAGAAGAAGCACCGGCTAACTCCGTGCCAGC |
| | | AGCCGCGGTAATACGGAGGGTGCAAGCGTTAATCGGAATTACTGGGC |
| | | GTAAAGCGCACGCAGGCGGTCTGTCAAGTCGGATGTGAAATCCCCGG |
| | | GCTCAACCTGGGAACTGCATTCGAAACTGGCAGGCTAGAGTCTTGTA |
| | | GAGGGGGGTAGAATTCCAGGTGTAGCGGTGAAATGCGTAGAGATCTG |
| | | GAGGAATACCGGTGGCGAAGGCGGCCCCCTGGACAAAGACTGACGCT |
| | | CAGGTGCGAAAGCGTGGGGAGCAAACAGGATTAGATACCCTGGTAGT |
| | | CCACGCCGTAAACGATGTCGACTTGGAGGTTGTGCCCTTGAGGCGTG |
| | | GCTTCCGGAGCTAACGCGTTAAGTCGACCGCCTGGGGAGTACGGCCG |
| | | CAAGGTTAAAACTCAAATGAATTGACGGGGGCCCGCACAAGCGGTGG |
| | | AGCATGTGGTTTAATTCGATGCAACGCGAAGAACCTTACCTACTCTTG |
| | | ACATCCACGGAATTTAGCAGAGATGCTTTAGTGCCTTCGGGAACCGT |
| | | GAGACAGGTGCTGCATGGCTGTCGTCAGCTCGTGTTGTGAAATGTTGG |
| | | GTTAAGTCCCGCAACGAGCGCAACCCTTATCCTTTGTTGCCAGCGGTT |
| | | CGGCCGGGAACTCAAAGGAGACTGCCAGTGATAAACTGGAGGAAGG |
| | | TGGGGATGACGTCAAGTCATCATGGCCCTTACGAGTAGGGCTACACA |
| | | CGTGCTACAATGGCATATACAAAGAGAAGCGACCTCGCGAGAGCAAG |
| | | CGGACCTCATAAAGTATGTCGTAGTCCGGATCGGAGTCTGCAACTCG |
| | | ACTCCGTGAAGTCGGAATCGCTAGTAATCGTAGATCAGAATGCTACG |
| | | GTGAATACGTTCCCGGGCCTTGTACACACCGCCCGTCACACCATGGG |
| | | AGTGGGTTGCAAAAGAAGTAGGTAGCTTAACCTTCGGGAGGGCGCTT |
| | | ACCACTTTGTGATTCATGACTGGGGTGAAGTCGTAACAAGGTAACCG |
| | | TAGGGGAACCTGCGGTTGGATCACCTCCTT |
| 51 | DP51 16S rRNA | TTGAAGAGTTTGATCATGGCTCAGATTGAACGCTGGCGGCAGGCCTA |
| | | ACACATGCAAGTCGAGCGGTAGCACAGGGAGCTTGCTCCTGGGTGAC |
| | | GAGCGGCGGACGGGTGAGTAATGTCTGGGAAACTGCCTGATGGAGGG |
| | | GGATAACTACTGGAAACGGTAGCTAATACCGCATAACGTCGCAAGAC |
| | | CAAAGAGGGGGACCTTCGGGCCTCTTGCCATCAGATGTGCCCAGATG |
| | | GGATTAGCTAGTAGGTGAGGTAATGGCTCACCTAGGCGACGATCCCT |
| | | AGCTGGTCTGAGAGGATGACCAGCCACACTGGAACTGAGACACGGTC |
| | | CAGACTCCTACGGGAGGCAGCAGTGGGGAATATTGCACAATGGGCGC |
| | | AAGCCTGATGCAGCCATGCCGCGTGTATGAAGAAGGCCTTCGGGTTG |
| | | TAAAGTACTTTCAGCGAGGAGGAAGGCATTAAGGTTAATAACCTTGG |
| | | TGATTGACGTTACTCGCAGAAGAAGCACCGGCTAACTCCGTGCCAGC |
| | | AGCCGCGGTAATACGGGGGGTGCAAGCGTTAATCGGAATTACTGGGC |
| | | GTAAAGCGCACGCAGGCGGTTTGTCAAGTCGGATGTGAAATCCCCGG |
| | | GCTCAACCTGGGAACTGCATTCGAAACGGGCAAGCTAGAGTCTTGTA |
| | | GAGGGGGGTAGAATTCCAGGTGTAGCGGTGAAATGCGTAGAGATCTG |
| | | GAGGAATACCGGTGGCGAAGGCGGCCCCCTGGACAAAGACTGACGCT |
| | | CAGGTGCGAAAGCGTGGGGAGCAAACAGGATTAGATACCCTGGTAGT |
| | | CCACGCCGTAAACGATGTCGACTTGGAGGTTGTGCCCTTGAGGCGTG |
| | | GCTTCCGGAGCTAACGCGTTAAGTCGACCGCCTGGGGAGTACGGCCG |
| | | CAAGGTTAAAACTCAAATGAATTGACGGGGGCCCGCACAAGCGGTGG |
| | | AGCATGTGGTTTAATTCGATGCAACGCGAAGAACCTTACCTACTCTTG |
| | | ACATCCAGAGAACTTTCCAGAGATGGATTGGTGCCTTCGGGAACTCT |
| | | GAGACAGGTGCTGCATGGCTGTCGTCAGCTCGTGTTGTGAAATGTTGG |
| | | GTTAAGTCCCGCAACGAGCGCAACCCTTATCCTTTGTTGCCAGCGAGT |
| | | AATGTCGGGAACTCAAAGGAGACTGCCAGTGACAAACTGGAGGAAG |
| | | GTGGGGATGACGTCAAGTCATCATGGCCCTTACGAGTAGGGCTACAC |
| | | ACGTGCTACAATGGCATATACAAAGAGAAGCGACCTCGCGAGAGCAA |
| | | GCGGACCTCACAAAGTATGTCGTAGTCCGGATCGGAGTCTGCAACTC |
| | | GACTCCGTGAAGTCGGAATCGCTAGTAATCGTAGATCAGAATGCTAC |
| | | GGTGAATACGTTCCCGGGCCTTGTACACACCGCCCGTCACACCATGG |
| | | GAGTGGGTTGCAAAAGAAGTAGGTAGCTTAACCTTCGGGAGGGCGCT |
| | | TACCACTTTGTGATTCATGACTGGGGTGAAGTCGTAACAAGGTAACC |
| | | GTAGGGGAACCTGCGGTTGGATCACCTCCTT |
| 52 | DP52 16S rRNA | ACGGAGAGTTTGATCCTGGCTCAGGATGAACGCTGGCGGCGTGCTTA |
| | | ACACATGCAAGTCGAACGATGATCCCAGCTTGCTGGGGGATTAGTGG |
| | | CGAACGGGTGAGTAACACGTGAGTAACCTGCCCTTGACTCTGGGATA |
| | | AGCCTGGGAAACTGGGTCTAATACCGGATATGACTGTCTGACGCATG |
| | | TCAGGTGGTGGAAAGCTTTTGTGGTTTTGGATGGACTCGCGGCCTATC |
| | | AGCTTGTTGGTGGGGTAATGGCCTACCAAGGCGACGACGGGTAGCCG |
| | | GCCTGAGAGGGTGACCGGCCACACTGGGACTGAGACACGGCCCAGAC |
| | | TCCTACGGGAGGCAGCAGTGGGGAATATTGCACAATGGGCGCAAGCC |
| | | TGATGCAGCGACGCCGCGTGAGGGATGACGGCCTTCGGGTTGTAAAC |
| | | CTCTTTCAGTAGGGAAGAAGCGAAAGTGACGGTACCTGCAGAAGAAG |
| | | CGCCGGCTAACTACGTGCCAGCAGCCGCGGTAATACGTAGGGCGCAA |
| | | GCGTTATCCGGAATTATTGGGCGTAAAGAGCTCGTAGGCGGTTTGTCG |
| | | CGTCTGCTGTGAAAGACCGGGGCTCAACTCCGGTTCTGCAGTGGGTA |
| | | CGGGCAGACTAGAGTGCAGTAGGGGAGACTGGAATTCCTGGTGTAGC |
| | | GGTGAAATGCGCAGATATCAGGAGGAACACCGATGGCGAAGGCAGG |
| | | TCTCTGGGCTGTAACTGACGCTGAGGAGCGAAAGCATGGGGAGCGAA |
| | | CAGGATTAGATACCCTGGTAGTCCATGCCGTAAACGTTGGGCACTAG |
| | | GTGTGGGGGACATTCCACGTTTTCCGCGCCGTAGCTAACGCATTAAGT |
| | | GCCCCGCCTGGGGAGTACGGCCGCAAGGCTAAAACTCAAAGGAATTG |
| | | ACGGGGGCCCGCACAAGCGGCGGAGCATGCGGATTAATTCGATGCAA |
| | | CGCGAAGAACCTTACCAAGGCTTGACATGAACCGGTAATACCTGGAA |
| | | ACAGGTGCCCCGCTTGCGGTCGGTTTACAGGTGGTGCATGGTTGTCGT |
| | | CAGCTCGTGTCGTGAGATGTTGGGTTAAGTCCCGCAACGAGCGCAAC |
| | | CCTCGTTCTATGTTGCCAGCGCGTTATGGCGGGGACTCATAGGAGACT |
| | | GCCGGGGTCAACTCGGAGGAAGGTGGGGACGACGTCAAATCATCATG |
| | | CCCCTTATGTCTTGGGCTTCACGCATGCTACAATGGCCGGTACAAAGG |
| | | GTTGCGATACTGTGAGGTGGAGCTAATCCCAAAAAGCCGGTCTCAGT |
| | | TCGGATTGGGGTCTGCAACTCGACCCCATGAAGTCGGAGTCGCTAGT |
| | | AATCGCAGATCAGCAACGCTGCGGTGAATACGTTCCCGGGCCTTGTA |
| | | CACACCGCCCGTCAAGTCACGAAAGTTGGTAACACCCGAAGCCGGTG |
| | | GCCTAACCCTTGTGGGGGGAGCCGTCGAAGGTGGGACCGGCGATTGG |
| | | GACTAAGTCGTAACAAGGTAGCCGTACCGGAAGGTGCGGCTGGATCA |
| | | CCTCCTTT |
| 53 | DP53 16S rRNA | TGAAGAGTTTGATCATGGCTCAGATTGAACGCTGGCGGCAGGCCTAA |
| | | CACATGCAAGTCGAGCGGTAGAGAGAAGCTTGCTTCTCTTGAGAGCG |
| | | GCGGACGGGTGAGTAATACCTAGGAATCTGCCTGATAGTGGGGGATA |
| | | ACGTTCGGAAACGGACGCTAATACCGCATACGTCCTACGGGAGAAAG |
| | | CAGGGGACCTTCGGGCCTTGCGCTATCAGATGAGCCTAGGTCGGATT |
| | | AGCTAGTTGGTGAGGTAATGGCTCACCAAGGCTACGATCCGTAACTG |
| | | GTCTGAGAGGATGATCAGTCACACTGGAACTGAGACACGGTCCAGAC |
| | | TCCTACGGGAGGCAGCAGTGGGGAATATTGGACAATGGGCGAAAGCC |
| | | TGATCCAGCCATGCCGCGTGTGTGAAGAAGGTCTTCGGATTGTAAAG |
| | | CACTTTAAGTTGGGAGGAAGGGCAGTTACCTAATACGTGATTGTCTTG |
| | | ACGTTACCGACAGAATAAGCACCGGCTAACTCTGTGCCAGCAGCCGC |
| | | GGTAATACAGAGGGTGCAAGCGTTAATCGGAATTACTGGGCGTAAAG |
| | | CGCGCGTAGGTGGTTTGTTAAGTTGAATGTGAAATCCCCGGGCTCAAC |
| | | CTGGGAACTGCATCCAAAACTGGCAAGCTAGAGTATGGTAGAGGGTA |
| | | GTGGAATTTCCTGTGTAGCGGTGAAATGCGTAGATATAGGAAGGAAC |
| | | ACCAGTGGCGAAGGCGACTACCTGGACTGATACTGACACTGAGGTGC |
| | | GAAAGCGTGGGGAGCAAACAGGATTAGATACCCTGGTAGTCCACGCC |
| | | GTAAACGATGTCAACTAGCCGTTGGGAGTCTTGAACTCTTAGTGGCGC |
| | | AGCTAACGCATTAAGTTGACCGCCTGGGGAGTACGGCCGCAAGGTTA |
| | | AAACTCAAATGAATTGACGGGGGCCCGCACAAGCGGTGGAGCATGTG |
| | | GTTTAATTCGAAGCAACGCGAAGAACCTTACCAGGCCTTGACATCCA |
| | | ATGAACTTTCTAGAGATAGATTGGTGCCTTCGGGAACATTGAGACAG |
| | | GTGCTGCATGGCTGTCGTCAGCTCGTGTCGTGAGATGTTGGGTTAAGT |
| | | CCCGTAACGAGCGCAACCCTTGTCCTTAGTTACCAGCACGTAATGGTG |
| | | GGCACTCTAAGGAGACTGCCGGTGACAAACCGGAGGAAGGTGGGGA |
| | | TGACGTCAAGTCATCATGGCCCTTACGGCCTGGGCTACACACGTGCTA |
| | | CAATGGTCGGTACAAAGGGTTGCCAAGCCGCGAGGTGGAGCTAATCC |
| | | CATAAAACCGATCGTAGTCCGGATCGCAGTCTGCAACTCGACTGCGT |
| | | GAAGTCGGAATCGCTAGTAATCGTGAATCAGAATGTCACGGTGAATA |
| | | CGTTCCCGGGCCTTGTACACACCGCCCGTCACACCATG |
| 54 | DP54 16S rRNA | CTTGAGAGTTTGATCCTGGCTCAGAGCGAACGCTGGCGGCAGGCTTA |
| | | ACACATGCAAGTCGAGCGGGCACCTTCGGGTGTCAGCGGCAGACGGG |
| | | TGAGTAACACGTGGGAACGTACCCTTCGGTTCGGAATAACGCTGGGA |
| | | AACTAGCGCTAATACCGGATACGCCCTTTTGGGGAAAGGTTTACTGCC |
| | | GAAGGATCGGCCCGCGTCTGATTAGCTAGTTGGTGGGGTAACGGCCT |
| | | ACCAAGGCGACGATCAGTAGCTGGTCTGAGAGGATGATCAGCCACAC |
| | | TGGGACTGAGACACGGCCCAGACTCCTACGGGAGGCAGCAGTGGGG |
| | | AATATTGGACAATGGGCGCAAGCCTGATCCAGCCATGCCGCGTGAGT |
| | | GATGAAGGCCTTAGGGTTGTAAAGCTCTTTTGTCCGGGACGATAATG |
| | | ACGGTACCGGAAGAATAAGCCCCGGCTAACTTCGTGCCAGCAGCCGC |
| | | GGTAATACGAAGGGGGCTAGCGTTGCTCGGAATCACTGGGCGTAAAG |
| | | GGCGCGTAGGCGGCCATTCAAGTCGGGGGTGAAAGCCTGTGGCTCAA |
| | | CCACAGAATTGCCTTCGATACTGTTTGGCTTGAGTTTGGTAGAGGTTG |
| | | GTGGAACTGCGAGTGTAGAGGTGAAATTCGTAGATATTCGCAAGAAC |
| | | ACCAGTGGCGAAGGCGGCCAACTGGACCAATACTGACGCTGAGGCGC |
| | | GAAAGCGTGGGGAGCAAACAGGATTAGATACCCTGGTAGTCCACGCC |
| | | GTAAACGATGAATGCTAGCTGTTGGGGTGCTTGCACCTCAGTAGCGC |
| | | AGCTAACGCTTTAAGCATTCCGCCTGGGGAGTACGGTCGCAAGATTA |
| | | AAACTCAAAGGAATTGACGGGGGCCCGCACAAGCGGTGGAGCATGT |
| | | GGTTTAATTCGAAGCAACGCGCAGAACCTTACCATCCCTTGACATGTC |
| | | GTGCCATCCGGAGAGATCCGGGGTTCCCTTCGGGGACGCGAACACAG |
| | | GTGCTGCATGGCTGTCGTCAGCTCGTGTCGTGAGATGTTGGGTTAAGT |
| | | CCCGCAACGAGCGCAACCCACGTCCTTAGTTGCCATCATTTAGTTGGG |
| | | CACTCTAGGGAGACTGCCGGTGATAAGCCGCGAGGAAGGTGTGGATG |
| | | ACGTC |
| 55 | DP55 16S rRNA | TCGGAGAGTTTGATCCTGGCTCAGGATGAACGCTGGCGGCGTGCCTA |
| | | ATACATGCAAGTCGAGCGAACTGATTAGAAGCTTGCTTCTATGACGTT |
| | | AGCGGCGGACGGGTGAGTAACACGTGGGCAACCTGCCTGTAAGACTG |
| | | GGATAACTTCGGGAAACCGAAGCTAATACCGGATAGGATCTTCTCCT |
| | | TCATGGGAGATGATTGAAAGATGGTTTCGGCTATCACTTACAGATGG |
| | | GCCCGCGGTGCATTAGCTAGTTGGTGAGGTAACGGCTCACCAAGGCA |
| | | ACGATGCATAGCCGACCTGAGAGGGTGATCGGCCACACTGGGACTGA |
| | | GACACGGCCCAGACTCCTACGGGAGGCAGCAGTAGGGAATCTTCCGC |
| | | AATGGACGAAAGTCTGACGGAGCAACGCCGCGTGAGTGATGAAGGCT |
| | | TTCGGGTCGTAAAACTCTGTTGTTAGGGAAGAACAAGTACAAGAGTA |
| | | ACTGCTTGTACCTTGACGGTACCTAACCAGAAAGCCACGGCTAACTA |
| | | CGTGCCAGCAGCCGCGGTAATACGTAGGTGGCAAGCGTTATCCGGAA |
| | | TTATTGGGCGTAAAGCGCGCGCAGGCGGTTTCTTAAGTCTGATGTGAA |
| | | AGCCCACGGCTCAACCGTGGAGGGTCATTGGAAACTGGGGAACTTGA |
| | | GTGCAGAAGAGAAAAGCGGAATTCCACGTGTAGCGGTGAAATGCGTA |
| | | GAGATGTGGAGGAACACCAGTGGCGAAGGCGGCTTTTTGGTCTGTAA |
| | | CTGACGCTGAGGCGCGAAAGCGTGGGGAGCAAACAGGATTAGATAC |
| | | CCTGGTAGTCCACGCCGTAAACGATGAGTGCTAAGTGTTAGAGGGTT |
| | | TCCGCCCTTTAGTGCTGCAGCTAACGCATTAAGCACTCCGCCTGGGGA |
| | | GTACGGTCGCAAGACTGAAACTCAAAGGAATTGACGGGGGCCCGCAC |
| | | AAGCGGTGGAGCATGTGGTTTAATTCGAAGCAACGCGAAGAACCTTA |
| | | CCAGGTCTTGACATCCTCTGACAACTCTAGAGATAGAGCGTTCCCCTT |
| | | CGGGGGACAGAGTGACAGGTGGTGCATGGTTGTCGTCAGCTCGTGTC |
| | | GTGAGATGTTGGGTTAAGTCCCGCAACGAGCGCAACCCTTGATCTTA |
| | | GTTGCCAGCATTTAGTTGGGCACTCTAAGGTGACTGCCGGTGACAAA |
| | | CCGGAGGAAGGTGGGGATGACGTCAAATCATCATGCCCCTTATGACC |
| | | TGGGCTACACACGTGCTACAATGGATGGTACAAAGGGCTGCAAGACC |
| | | GCGAGGTCAAGCCAATCCCATAAAACCATTCTCAGTTCGGATTGTAG |
| | | GCTGCAACTCGCCTACATGAAGCTGGAATCGCTAGTAATCGCGGATC |
| | | AGCATGCT |
| 56 | DP56 16S rRNA | ATTGGAGAGTTTGATCCTGGCTCAGGACGAACGCTGGCGGCGTGCCT |
| | | AATACATGCAAGTCGAGCGGACCTGATGGAGTGCTTGCACTCCTGAT |
| | | GGTTAGCGGCGGACGGGTGAGTAACACGTAGGCAACCTGCCCTCAAG |
| | | ACTGGGATAACTACCGGAAACGGTAGCTAATACCGGATAATTTATTT |
| | | CACAGCATTGTGGAATAATGAAAGACGGAGCAATCTGTCACTTGGGG |
| | | ATGGGCCTGCGGCGCATTAGCTAGTTGGTGGGGTAACGGCTCACCAA |
| | | GGCGACGATGCGTAGCCGACCTGAGAGGGTGAACGGCCACACTGGG |
| | | ACTGAGACACGGCCCAGACTCCTACGGGAGGCAGCAGTAGGGAATCT |
| | | TCCGCAATGGGCGAAAGCCTGACGGAGCAACGCCGCGTGAGTGATGA |
| | | AGGTTtTCGGATCGTAAAGCTCTGTTGCCAAGGAAGAACGTCTTCTAG |
| | | AGTAACTGCTAGGAGAGTGACGGTACTTGAGAAGAAAGCCCCGGCTA |
| | | ACTACGTGCCAGCAGCCGCGGTAATACGTAGGGGGCAAGCGTTGTCC |
| | | GGAATTATTGGGCGTAAAGCGCGCGCAGGCGGTTCTTTAAGTCTGGT |
| | | GTTTAAACCCGAGGCTCAACTTCGGGTCGCACTGGAAACTGGGGAAC |
| | | TTGAGTGCAGAAGAGGAGAGTGGAATTCCACGTGTAGCGGTGAAATG |
| | | CGTAGATATGTGGAGGAACACCAGTGGCGAAGGCGACTCTCTGGGCT |
| | | GTAACTGACGCTGAGGCGCGAAAGCGTGGGGAGCAAACAGGATTAG |
| | | ATACCCTGGTAGTCCACGCCGTAAACGATGAATGCTAGGTGTTAGGG |
| | | GTTTCGATACCCTTGGTGCCGAAGTTAACACATTAAGCATTCCGCCTG |
| | | GGGAGTACGGTCGCAAGACTGAAACTCAAAGGAATTGACGGGGACC |
| | | CGCACAAGCAGTGGAGTATGTGGTTTAATTCGAAGCAACGCGAAGAA |
| | | CCTTACCAAGTCTTGACATCCCTCTGAATCCTCTAGAGATAGAGGCGG |
| | | CCTTCGGGACAGAGGTGACAGGTGGTGCATGGTTGTCGTCAGCTCGT |
| | | GTCGTGAGATGTTGGGTTAAGTCCCGCAACGAGCGCAACCCTTGATTT |
| | | TAGTTGCCAGCACATCATGGTGGGCACTCTAGAATGACTGCCGGTGA |
| | | CAAACCGGAGGAAGGCGGGGATGACGTCAAATCATCATGCCCCTTAT |
| | | GACTTGGGCTACACACGTACTACAATGGCTGGTACAACGGGAAGCGA |
| | | AGCCGCGAGGTGGAGCCAATCCTATAAAAGCCAGTCTCAGTTCGGAT |
| | | TGCAGGCTGCAACTCGCCTGCATGAAGTCGGAATTGCTAGTAATCGC |
| | | GGATCAGCATGCCGCGGTGAATACGTTCCCGGGTCTTGTACACACCG |
| | | CCCGTCACACCACGAGAGTTTACAACACCCGAAGTCGGTGGGGTAAC |
| | | CCGCAAGGGAGCCAGCCGCCGAAGGTGGGGTAGATGATTGGGGTGA |
| | | AGTCGTAACAAGGTAGCCGTATCGGAAGGTGCGGCTGGATCACCTCC |
| | | TTT |
| 57 | DP57 16S rRNA | ATTGGAGAGTTTGATCCTGGCTCAGGATGAACGCTGGCGGCGTGCCT |
| | | AATACATGCAAGTCGAGCGAATGGATTAAGAGCTTGCTCTTATGAAG |
| | | TTAGCGGCGGACGGGTGAGTAACACGTGGGTAACCTGCCCATAAGAC |
| | | TGGGATAACTCCGGGAAACCGGGGCTAATACCGGATAACATTTTGCA |
| | | CCGCATGGTGCGAAATTCAAAGGCGGCTTCGGCTGTCACTTATGGAT |
| | | GGACCCGCGTCGCATTAGCTAGTTGGTGAGGTAACGGCTCACCAAGG |
| | | CAACGATGCGTAGCCGACCTGAGAGGGTGATCGGCCACACTGGGACT |
| | | GAGACACGGCCCAGACTCCTACGGGAGGCAGCAGTAGGGAATCTTCC |
| | | GCAATGGACGAAAGTCTGACGGAGCAACGCCGCGTGAGTGATGAAG |
| | | GCTTTCGGGTCGTAAAACTCTGTTGTTAGGGAAGAACAAGTGCTAGTT |
| | | GAATAAGCTGGCACCTTGACGGTACCTAACCAGAAAGCCACGGCTAA |
| | | CTACGTGCCAGCAGCCGCGGTAATACGTAGGTGGCAAGCGTTATCCG |
| | | GAATTATTGGGCGTAAAGCGCGCGCAGGTGGTTTCTTAAGTCTGATGT |
| | | GAAAGCCCACGGCTCAACCGTGGAGGGTCATTGGAAACTGGGAGACT |
| | | TGAGTGCAGAAGAGGAAAGTGGAATTCCATGTGTAGCGGTGAAATGC |
| | | GTAGAGATATGGAGGAACACCAGTGGCGAAGGCGACTTTCTGGTCTG |
| | | TAACTGACACTGAGGCGCGAAAGCGTGGGGAGCAAACAGGATTAGA |
| | | TACCCTGGTAGTCCACGCCGTAAACGATGAGTGCTAAGTGTTAGAGG |
| | | GTTTCCGCCCTTTAGTGCTGAAGTTAACGCATTAAGCACTCCGCCTGG |
| | | GGAGTACGGCCGCAAGGCTGAAACTCAAAGGAATTGACGGGGGCCC |
| | | GCACAAGCGGTGGAGCATGTGGTTTAATTCGAAGCAACGCGAAGAAC |
| | | CTTACCAGGTCTTGACATCCTCTGACAACCCTAGAGATAGGGCTTCCC |
| | | CTTCGGGGGCAGAGTGACAGGTGGTGCATGGTTGTCGTCAGCTCGTG |
| | | TCGTGAGATGTTGGGTTAAGTCCCGCAACGAGCGCAACCCTTGATCTT |
| | | AGTTGCCATCATTAAGTTGGGCACTCTAAGGTGACTGCCGGTGACAA |
| | | ACCGGAGGAAGGTGGGGATGACGTCAAATCATCATGCCCCTTATGAC |
| | | CTGGGCTACACACGTGCTACAATGGACGGTACAAAGAGCTGCAAGAC |
| | | CGCGAGGTGGAGCTAATCTCATAAAACCGTTCTCAGTTCGGATTGTAG |
| | | GCTGCAACTCGCCTACATGAAGCTGGAATCGCTAGTAATCGCGGATC |
| | | AGCATGCCGCGGTGAATACGTTCCCGGGCCTTGTACACACCGCCCGTC |
| | | ACACCACGAGAGTTTGTAACACCCGAAGTCGGTGGGGTAACCTTTTT |
| | | GGAGCCAGCCGCCTAAGGTGGGACAGATGATTGGGGTGAAGTCGTAA |
| | | CAAGGTAGCCGTATCGGAAGGTGCGGCTGGATCACCTCCTTT |
| 58 | DP58 16S rRNA | AATGACGGTACCTGAAGAATAAGCACCGGCTAACTACGTGCCAGCAG |
| | | CCGCGGTAATACGTAGGGTGCAAGCGTTAATCGGAATTACTGGGCGT |
| | | AAAGCGTGCGCAGGCGGTTTTGTAAGTCTGATGTGAAATCCCCGGGC |
| | | TCAACCTGGGAATTGCATTGGAGACTGCAAGGCTAGAATCTGGCAGA |
| | | GGGGGGTAGAATTCCACGTGTAGCAGTGAAATGCGTAGATATGTGGA |
| | | GGAACACCGATGGCGAAGGCAGCCCCCTGGGTCAAGATTGACGCTCA |
| | | TGCACGAAAGCGTGGGGAGCAAACAGGATTAGATACCCTGGTAGTCC |
| | | ACGCCCTAAACGATGTCTACTAGTTGTCGGGTCTTAATTGACTTGGTA |
| | | ACGCAGCTAACGCGTGAAGTAGACCGCCTGGGGAGTACGGTCGCAAG |
| | | ATTAAAACTCAAAGGAATTGACGGGGACCCGCACAAGCGGTGGATGA |
| | | TGTGGATTAATTCGATGCAACGCGAAAAACCTTACCTACCCTTGACAT |
| | | GGCTGGAATCCTCGAGAGATTGGGGAGTGCTCGAAAGAGAACCAGTA |
| | | CACAGGTGCTGCATGGCTGTCGTCAGCTCGTGTCGTGAGATGTTGGGT |
| | | TAAGTCCCGCAACGAGCGCAACCCTTGTCATTAGTTGCTACGAAAGG |
| | | GCACTCTAATGAGACTGCCGGTGACAAACCGGAGGAAGGTGGGGATG |
| | | ACGTCAAGTCCTCATGGCCCTTATGGGTAGGGCTTCACACGTCATACA |
| | | ATGGTACATACAGAGCGCCGCCAACCCGCGAGGGGGAGCTAATCGCA |
| | | GAAAGTGTATCGTAGTCCGGATTGTAGTCTGCAACTCGACTGCATGA |
| | | AGTTGGAATCGCTAGTAATCGCGGATCAGCATGTCGCGGTGAATACG |
| | | TTCCCGGGTCTTGTACACACCGCCCGTCACACCATGGGAGCGGGTTTT |
| | | ACCAGAAGTAGGTAGCTTAACCGTAAGGAGGGCGCTTACCACGGTAG |
| | | GATTCGTGACTGGGGTGAAGTCGTAACAAGGTAGCCGTATCGGAAGG |
| | | TGCGGCTGGATCACCTCCTTT |
| 59 | DP59 16S rRNA | TTGAAGAGTTTGATCATGGCTCAGATTGAACGCTGGCGGCAGGCCTA |
| | | ACACATGCAAGTCGAACGGTAACAGGAAGCAGCTTGCTGCTTTGCTG |
| | | ACGAGTGGCGGACGGGTGAGTAATGTCTGGGAAACTGCCTGATGGAG |
| | | GGGGATAACTACTGGAAACGGTAGCTAATACCGCATAACGTCGCAAG |
| | | ACCAAAGAGGGGGACCTTCGGGCCTCTTGCCATCAGATGTGCCCAGA |
| | | TGGGATTAGCTAGTAGGTGGGGTAACGGCTCACCTAGGCGACGATCC |
| | | CTAGCTGGTCTGAGAGGATGACCAGCCACACTGGAACTGAGACACGG |
| | | TCCAGACTCCTACGGGAGGCAGCAGTGGGGAATATTGCACAATGGGC |
| | | GCAAGCCTGATGCAGCCATGCCGCGTGTATGAAGAAGGCCTTCGGGT |
| | | TGTAAAGTACTTTCAGCGGGGAGGAAGGCGATGCGGTTAATAACCGC |
| | | GTCGATTGACGTTACCCGCAGAAGAAGCACCGGCTAACTCCGTGCCA |
| | | GCAGCCGCGGTAATACGGAGGGTGCAAGCGTTAATCGGAATTACTGG |
| | | GCGTAAAGCGCACGCAGGCGGTCTGTCAAGTCGGATGTGAAATCCCC |
| | | GGGCTCAACCTGGGAACTGCATCCGAAACTGGCAGGCTTGAGTCTCG |
| | | TAGAGGGGGGTAGAATTCCAGGTGTAGCGGTGAAATGCGTAGAGATC |
| | | TGGAGGAATACCGGTGGCGAAGGCGGCCCCCTGGACGAAGACTGAC |
| | | GCTCAGGTGCGAAAGCGTGGGGAGCAAACAGGATTAGATACCCTGGT |
| | | AGTCCACGCCGTAAACGATGTCGACTTGGAGGTTGTGCCCTTGAGGC |
| | | GTGGCTTCCGGAGCTAACGCGTTAAGTCGACCGCCTGGGGAGTACGG |
| | | CCGCAAGGTTAAAACTCAAATGAATTGACGGGGGCCCGCACAAGCGG |
| | | TGGAGCATGTGGTTTAATTCGATGCAACGCGAAGAACCTTACCTGGTC |
| | | TTGACATCCACAGAACTTGGCAGAGATGCCTTGGTGCCTTCGGGAACT |
| | | GTGAGACAGGTGCTGCATGGCTGTCGTCAGCTCGTGTTGTGAAATGTT |
| | | GGGTTAAGTCCCGCAACGAGCGCAACCCTTATCCTTTGTTGCCAGCGG |
| | | TTAGGCCGGGAACTCAAAGGAGACTGCCAGTGATAAACTGGAGGAA |
| | | GGTGGGGATGACGTCAAGTCATCATGGCCCTTACGACCAGGGCTACA |
| | | CACGTGCTACAATGGCGCATACAAAGAGAAGCGATCTCGCGAGAGCC |
| | | AGCGGACCTCATAAAGTGCGTCGTAGTCCGGATTGGAGTCTGCAACT |
| | | CGACTCCATGAAGTCGGAATCGCTAGTAATCGTGAATCAGAATGTCA |
| | | CGGTGAATACGTTCCCGGGCCTTGTACACACCGCCCGTCACACCATGG |
| | | GAGTGGGTTGCAAAAGAAGTAGGTAGCTTAACCTTCGGGAGGGCGCT |
| | | TACCACTTTGTGATTCATGACTGGGGTGAAGTCGTAACAAGGTAACC |
| | | GTAGGGGAACCTGCGGTTGGATCACCTCCTT |
| 60 | DP60 16S rRNA | TCGGAGAGTTTGATCCTGGCTCAGGACGAACGCTGGCGGCGTGCCTA |
| | | ATACATGCAAGTCGAGCGAATCGATGGGAGCTTGCTCCCTGAGATTA |
| | | GCGGCGGACGGGTGAGTAACACGTGGGCAACCTGCCTATAAGACTGG |
| | | GATAACTTCGGGAAACCGGAGCTAATACCGGATACGTTCTTTTCTCGC |
| | | ATGAGAGAAGATGGAAAGACGGTTTTGCTGTCACTTATAGATGGGCC |
| | | CGCGGCGCATTAGCTAGTTGGTGAGGTAATGGCTCACCAAGGCGACG |
| | | ATGCGTAGCCGACCTGAGAGGGTGATCGGCCACACTGGGACTGAGAC |
| | | ACGGCCCAGACTCCTACGGGAGGCAGCAGTAGGGAATCTTCCGCAAT |
| | | GGACGAAAGTCTGACGGAGCAACGCCGCGTGAACGAAGAAGGCCTT |
| | | CGGGTCGTAAAGTTCTGTTGTTAGGGAAGAACAAGTACCAGAGTAAC |
| | | TGCTGGTACCTTGACGGTACCTAACCAGAAAGCCACGGCTAACTACG |
| | | TGCCAGCAGCCGCGGTAATACGTAGGTGGCAAGCGTTGTCCGGAATT |
| | | ATTGGGCGTAAAGCGCGCGCAGGTGGTTCCTTAAGTCTGATGTGAAA |
| | | GCCCACGGCTCAACCGTGGAGGGTCATTGGAAACTGGGGAACTTGAG |
| | | TGCAGAAGAGGAAAGTGGAATTCCAAGTGTAGCGGTGAAATGCGTAG |
| | | AGATTTGGAGGAACACCAGTGGCGAAGGCGACTTTCTGGTCTGTAAC |
| | | TGACACTGAGGCGCGAAAGCGTGGGGAGCAAACAGGATTAGATACC |
| | | CTGGTAGTCCACGCCGTAAACGATGAGTGCTAAGTGTTAGAGGGTTT |
| | | CCGCCCTTTAGTGCTGCAGCTAACGCATTAAGCACTCCGCCTGGGGAG |
| | | TACGGCCGCAAGGCTGAAACTCAAAGGAATTGACGGGGGCCCGCACA |
| | | AGCGGTGGAGCATGTGGTTTAATTCGAAGCAACGCGAAGAACCTTAC |
| | | CAGGTCTTGACATCCTCTGACAACCCTAGAGATAGGGCGTTCCCCTTC |
| | | GGGGGACAGAGTGACAGGTGGTGCATGGTTGTCGTCAGCTCGTGTCG |
| | | TGAGATGTTGGGTTAAGTCCCGCAACGAGCGCAACCCTTGATCTTAGT |
| | | TGCCAGCATTCAGTTGGGCACTCTAAGGTGACTGCCGGTGACAAACC |
| | | GGAGGAAGGTGGGGATGACGTCAAATCATCATGCCCCTTATGACCTG |
| | | GGCTACACACGTGCTACAATGGATGGTACAAAGGGCTGCAAACCTGC |
| | | GAAGGTAAGCGAATCCCATAAAGCCATTCTCAGTTCGGATTGTAGGC |
| | | TGCAACTCGCCTACATGAAGCCGGAATCGCTAGTAATCGCGGATCAG |
| | | CATGCCGCGGTGAATACGTTCCCGGGCCTTGTACACACCGCCCGTCAC |
| | | ACCACGAGAGTTTGTAACACCCGAAGTCGGTGAGGTAACCTTTATGG |
| | | AGCCAGCCGCCTAAGGTGGGACAGATGATTGGGGTGAAGTCGTAACA |
| | | AGGTAGCCGTATCGGAAGGTGCGGCTGGATCACCTCCTTT |
| 61 | DP61 16S rRNA | GGAAGGCGGTCTGTCAAGTCGGATGTGAAATCCCCGGGCTCAACCTG |
| | | GGAACTGCATTCGAAACTGGCAGGCTAGAGTCTTGTAGAGGGGGGTA |
| | | GAATTCCAGGTGTAGCGGTGAAATGCGTAGAGATCTGGAGGAATACC |
| | | GGTGGCGAAGGCGGCCCCCTGGACAAAGACTGACGCTCAGGTGCGAA |
| | | AGCGTGGGGAGCAAACAGGATTAGATACCCTGGTAGTCCACGCCGTA |
| | | AACGATGTCGACTTGGAGGTTGTTCCCTTGAGGAGTGGCTTCCGGAGC |
| | | TAACGCGTTAAGTCGACCGCCTGGGGAGTACGGCCGCAAGGTTAAAA |
| | | CTCAAATGAATTGACGGGGGCCCGCACAAGCGGTGGAGCATGTGGTT |
| | | TAATTCGATGCAACGCGAAGAACCTTACCTACTCTTGACATCCACGGA |
| | | ATTTAGCAGAGATGCTTTAGTGCCTTCGGGAACCGTGAGACAGGTGC |
| | | TGCATGGCTGTCGTCAGCTCGTGTTGTGAAATGTTGGGTTAAGTCCCG |
| | | CAACGAGCGCAACCCTTATCCTTTGTTGCCAGCGGTCCGGCCGGGAA |
| | | CTCAAAGGAGACTGCCAGTGATAAACTGGAGGAAGGTGGGGATGAC |
| | | GTCAAGTCATCATGGCCCTTACGAGTAGGGCTACACACGTGCTACAA |
| | | TGGCGCATACAAAGAGAAGCGACCTCGCGAGAGCAAGCGGACCTCAT |
| | | AAAGTGCGTCGTAGTCCGGATCGGAGTCTGCAACTCGACTCCGTGAA |
| | | GTCGGAATCGCTAGTAATCGTAGATCAGAATGCTACGGTGAATACGT |
| | | TCCCGGGCCTTGTACACACCGCCCGTCACACCATGGGAGTGGGTTGC |
| | | AAAAGAAGTAGGTAGCTTAACCTTCGGGAGGGCGCTTACCACTTTGT |
| | | GATTCATGACTGGGGTGAAGTCGTAACAAGGTAACCGTAGGGGAACC |
| | | TGCGGTTGGATCACCTCCTT |
| 62 | DP62 16S rRNA | TGGCTCAGATTGAACGCTGGCGGCAGGCCTAACACATGCAAGTCGAA |
| | | CGGTAGCACAGAGGAGCTTGCTCCTTGGGTGACGAGTGGCGGACGGG |
| | | TGAGTAATGTCTGGGAAACTGCCCGATGGAGGGGGATAACTACTGGA |
| | | AACGGTAGCTAATACCGCATAACGTCTTCGGACCAAAGTGGGGGACC |
| | | TTCGGGCCTCACACCATCGGATGTGCCCAGATGGGATTAGCTAGTAG |
| | | GTGGGGTAATGGCTCACCTAGGCGACGATCCCTAGCTGGTCTGAGAG |
| | | GATGACCAGCCACACTGGAACTGAGACACGGTCCAGACTCCTACGGG |
| | | AGGCAGCAGTGGGGAATATTGCACAATGGGCGCAAGCCTGATGCAGC |
| | | CATGCCGCGTGTATGAAGAAGGCCTTCGGGTTGTAAAGTACTTTCAGT |
| | | GGGGAGGAAGGCGTTAAGGTTAATAACCTTGGCGATTGACGTTACCC |
| | | GCAGAAGAAGCACCGGCTAACTCCGTGCCAGCAGCCGCGGTAATACG |
| | | GAGGGTGCAAGCGTTAATCGGAATTACTGGGCGTAAAGCGCACGCAG |
| | | GCGGTCTGTCAAGTCGGATGTGAAATCCCCGGGCTCAACCTGGGAAC |
| | | TGCATTCGAAACTGGCAGGCTAGAGTCTTGTAGAGGGGGGTAGAATT |
| | | CCAGGTGTAGCGGTGAAATGCGTAGAGATCTGGAGGAATACCGGTGG |
| | | CGAAGGCGGCCCCCTGGACAAAGACTGACGCTCAGGTGCGAAAGCGT |
| | | GGGGAGCAAACAGGATTAGATACCCTGGTAGTCCACGCCGTAAACGA |
| | | TGTCGACTTGGAGGTTGTTCCCTTGAGGAGTGGCTTCCGGAGCTAACG |
| | | CGTTAAGTCGACCGCCTGGGGAGTACGG |
| 63 | DP63 16S rRNA | TGAAGAGTTTGATCATGGCTCAGATTGAACGCTGGCGGCAGGCCTAA |
| | | CACATGCAAGTCGAGCGGTAGAGAGAAGCTTGCTTCTCTTGAGAGCG |
| | | GCGGACGGGTGAGTAATGCCTAGGAATCTGCCTGGTAGTGGGGGATA |
| | | ACGTTCGGAAACGGACGCTAATACCGCATACGTCCTACGGGAGAAAG |
| | | CAGGGGACCTTCGGGCCTTGCGCTATCAGATGAGCCTAGGTCGGATT |
| | | AGCTAGTTGGTGAGGTAATGGCTCACCAAGGCGACGATCCGTAACTG |
| | | GTCTGAGAGGATGATCAGTCACACTGGAACTGAGACACGGTCCAGAC |
| | | TCCTACGGGAGGCAGCAGTGGGGAATATTGGACAATGGGCGAAAGCC |
| | | TGATCCAGCCATGCCGCGTGTGTGAAGAAGGTCTTCGGATTGTAAAG |
| | | CACTTTAAGTTGGGAGGAAGGGTTGTAGATTAATACTCTGCAATTTTG |
| | | ACGTTACCGACAGAATAAGCACCGGCTAACTCTGTGCCAGCAGCCGC |
| | | GGTAATACAGAGGGTGCAAGCGTTAATCGGAATTACTGGGCGTAAAG |
| | | CGCGCGTAGGTGGTTTGTTAAGTTGGATGTGAAATCCCCGGGCTCAAC |
| | | CTGGGAACTGCATTCAAAACTGACTGACTAGAGTATGGTAGAGGGTG |
| | | GTGGAATTTCCTGTGTAGCGGTGAAATGCGTAGATATAGGAAGGAAC |
| | | ACCAGTGGCGAAGGCGACCACCTGGACTAATACTGACACTGAGGTGC |
| | | GAAAGCGTGGGGAGCAAACAGGATTAGATACCCTGGTAGTCCACGCC |
| | | GTAAACGATGTCAACTAGCCGTTGGAAGCCTTGAGCTTTTAGTGGCGC |
| | | AGCTAACGCATTAAGTTGACCGCCTGGGGAGTACGGCCGCAAGGTTA |
| | | AAACTCAAATGAATTGACGGGGGCCCGCACAAGCGGTGGAGCATGTG |
| | | GTTTAATTCGAAGCAACGCGAAGAACCTTACCAGGCCTTGACATCCA |
| | | ATGAACTTTCTAGAGATAGATTGGTGCCTTCGGGAACATTGAGACAG |
| | | GTGCTGCATGGCTGTCGTCAGCTCGTGTCGTGAGATGTTGGGTTAAGT |
| | | CCCGTAACGAGCGCAACCCTTGTTCTTAGTTACCAGCACGTTATGGTG |
| | | GGCACTCTAAGGAGACTGCCGGTGACAAACCGGAGGAAGGTGGGGA |
| | | TGACGTCAAGTCATCATGGCCCTTACGGCCTGGGCTACACACGTGCTA |
| | | CAATGGTCGGTACAGAGGGTTGCCAAGCCGCGAGGTGGAGCTAATCC |
| | | CATAAAACCGATCGTAGTCCGGATCGCAGTCTGCAACTCGACTGCGT |
| | | GAAGTCGGAATCGCTAGTAATCGCGAATCAGAATGTCGCGGTGAATA |
| | | CGTTCCCGGGCCTTGTACACACCGCCCGTCACACCATGGGAGTGGGTT |
| | | GCACCAGAAGTAGCTAGTCTAACCTTCGGGAGGACGGTTACCACGGT |
| | | GTGATTCATGACTGGGGTGAAGTCGTAACAAGGTAGCCGTAGGGGAA |
| | | CCTGCGGCTGGATCACCTCCTT |
| 64 | DP64 ITS | TCCGTAGGTGAACCTGCGGAAGGATCATTAAATAATCAATAATTTTG |
| | sequence | GCTTGTCCATTATTATCTATTTACTGTGAACTGTATTATTACTTGACGC |
| | | TTGAGGGATGCTCCACTGCTATAAGGATAGGCGGTGGGGATGTTAAC |
| | | CGAGTCATAGTCAAGCTTAGGCTTGGTATCCTATTATTATTTACCAAA |
| | | AGAATTCAGAATTAATATTGTAACATAGACCTAAAAAATCTATAAAA |
| | | CAACTTTTAACAACGGATCTCTTGGTTCTCGCATCGATGAAGAACGTA |
| | | GCAAAGTGCGATAACTAGTGTGAATTGCATATTCAGTGAATCATCGA |
| | | GTCTTTGAACGCAACTTGCGCTCATTGGTATTCCAATGAGCACGCCTG |
| | | TTTCAGTATCAAAACAAACCCTCTATTCAATATTTTTGTTGAATAGGA |
| | | ATACTGAGAGTCTCTTGATCTTTTCTGATCTCGAACCTCTTGAAATGT |
| | | ACAAAGGCCTGATCTTGTTTGAATGCCTGAACTTTTTTTTAATATAAA |
| | | GAGAAGCTCTTGCGGTAAACTGTGCTGGGGCCTCCCAAATAATACTCT |
| | | TTTTAAATTTGATCTGAAATCAGGCGGGATTACCCGCTGAACTTAAGC |
| | | ATATCAATAAGCGGAGGAAAAGAAAATAACAATGATTTCCCTAGTAA |
| | | CGGCGAGTGAAGAGGAAAGAGCTCAAAGTTGGAAACTGTTTGGCTTA |
| | | GCTAAACCGTATTGTAAACTGTAGAAACATTTTCCTGGCACGCCGGAT |
| | | TAATAAGTCCTTTGGAACAAGGCATCATGGAGGGTGAGAATCCCGTC |
| | | TTTGATCCGAGTAGTTGTCTTTTGTGATATGTTTTCAAAGAGTCAGGTT |
| | | GTTTGGGAATGCAGCCTAAATTGGGTGGTAAATCTCACCTAAAGCTA |
| | | AATATTTGCGAGAGACCGATAGCGAACAAGTACCGTGAGGGAAAGAT |
| | | GAAAAGAACTTTGAAAAGAGAGTTAAACAGTATGTGAAATTGTTAAA |
| | | AGGGAACCGTTTGGAGCCAGACTGGTTTGACTGTAATCAACCTAGAA |
| | | TTCGTTCTGGGTGCACTTGCAGTCTATACCTGCCAACAACAGTTTGAT |
| | | TTGGAGGAAAAAATTAGTAGGAATGTAGCCTCTCGAGGTGTTATAGC |
| | | CTACTATCATACTCTGGATTGGACTGAGGAACGCAGCGAATGCCATT |
| | | AGGCGAGATTGCTGGGTGCTTTCGCTAATAAATGTTAGAATTTCTGCT |
| | | TCGGGTGGTGCTAATGTTTAAAGGAGGAACACATCTAGTATATTTTTT |
| | | ATTCGCTTAGGTTGTTGGCTTAATGACTCTAAATGACCCGTCTTGAAA |
| | | CACGGACCAAGGAGTCCACCATAAGTGCAAGTATTTGAGTGACAAAC |
| | | TCATATGCGTAAGGAAACTGATTGATACGAAATCTTTTGATGGCAGTA |
| | | TCACCCGGCGTTGACGTTTTATACTGAACTGACCGAGGTAAAGCACTT |
| | | ATGATGGGACCCGAAAGATGGTGAACTATGCCTGAATAGGGTGAAGC |
| | | CAGAGGAAACTCTGGTGGAGGCTCGTAGCGATTCTGACGTGCAAATC |
| | | GATCGTCAAATTTGGGTATAGGGGCGAAAGACTAATCGAACCATCTA |
| | | GTAGCTGGTTCCTGCCGAAGTTTCCCTCAGGA |
| 65 | DP65 ITS | TCCGTAGGTGAACCTGCGGAAGGATCATTATTGAAAACAAGGGTGTC |
| | sequence | CAATTTAACTTGGAACCCGAACTTCTCAATTCTAACTTTGTGCATCTG |
| | | TATTATGGCGAGCAGTCTTCGGATTGTGAGCCTTCACTTATAAACACT |
| | | AGTCTATGAATGTAAAATTTTTATAACAAATAAAAACTTTCAACAACG |
| | | GATCTCTTGGCTCTCGCATCGATGAAGAACGCAGCGAAATGCGATAC |
| | | GTAATGTGAATTGCAGAATTCAGTGAATCATCGAATCTTTGAACGCAT |
| | | CTTGCGCTCTCTGGTATTCCGGAGAGCATGTCTGTTTGAGTGTCATGA |
| | | ATTCTTCAACCCAATCTTTTCTTGTAATCGATTGGTGTTTGGATTTTGA |
| | | GCGCTGCTGGCTTCGGCCTAGCTCGTTCGTAATACATTAGCATCCCTA |
| | | ATACAAGTTTGGATTGACTTGGCGTAATAGACTATTCGCTAAGGATTC |
| | | GGTGGAAACATCGAGCCAACTTCATTAAGGAAGCTCCTAATTTAAAA |
| | | GTCTACCTTTTGATTAGATCTCAAATCAGGCAGGATTACCCGCTGAAC |
| | | TTAAGCATATCAATAAGCGGAGGAAAAGAAACTAACAAGGATTCCCC |
| | | TAGTAGCGGCGAGCGAAGCGGGAAAAGCTCAAATTTGTAATCTGGCG |
| | | TCTTCGACGTCCGAGTTGTAATCTCGAGAAGTGTTTTCCGTGATAGAC |
| | | CGCATACAAGTCTCTTGGAACAGAGCGTCATAGTGGTGAGAACCCAG |
| | | TACACGATGCGGATGCCTATTACTTTGTGATACACTTTCGAAGAGTCG |
| | | AGTTGTTTGGGAATGCAGCTCAAATTGGGTGGTAAATTCCATCTAAAG |
| | | CTAAATATTGGCGAGAGACCGATAGCGAACAAGTACCGTAAGGGAA |
| | | AGATGAAAAGCACTTTGGAAAGAGAGTTAACAGTACGTGAAATTGTT |
| | | GGAAGGGAAACACATGCAGTGATACTTGCTATTCGGGGCAACTCGAT |
| | | TGGCAGGCCCGCATCAGTTTTTCGGGGCGGAAAAGCGTAGAGAGAAG |
| | | GTAGCAATTTCGGTTGTGTTATAGCTCTTTACTGGATTCGCCCTGGGG |
| | | GACTGAGGAACGCAGCGTGCTTTTAGCAATTCCTTCGGGAATTCCACG |
| | | CTTAGGATGCGGGTTTATGGCTGTATATGACCCGTCTTGAAACACGGA |
| | | CCAAGGAGTCTAACATGCTTGCGAGTATTTGGGTGTCAAACCCGGAT |
| | | GCGCAATGAAAGTGAATGGAGGTGGGAAGCGCAAGCTGCACCATCG |
| | | ACCGATCTGGATTTTTTAAGATGGATTTGAGTAAGAGCAAGTATGTTG |
| | | GGACCCGAAAGATGGTGAACTATGCCTGAATAGGGCGAAGCCAGAG |
| | | GAAACTCTGGTGGAGGCTCGTAGCGGTTCTGACGTGCAAATCGATCG |
| | | TCAAATTTGGGTATAGGGGCGAAAGACTAATCGAACCATCTAGTAGC |
| | | TGGTTCCTGCCGAAGTTTCCCTCAGGA |
| 66 | DP66 ITS | TCCGTAGGTGAACCTGCGGAAGGATCATTACTGTGATTTATCCACCAC |
| | sequence | ACTGCGTGGGCGACACGAAACACCGAAACCGAACGCACGCCGTCAA |
| | | GCAAGAAATCCACAAAACTTTCAACAACGGATCTCTTGGTTCTCGCAT |
| | | CGATGAAGAGCGCAGCGAAATGCGATACCTAGTGTGAATTGCAGCCA |
| | | TCGTGAATCATCGAGTTCTTGAACGCACATTGCGCCCGCTGGTATTCC |
| | | GGCGGGCATGCCTGTCTGAGCGTCGTTTCCTTCTTGGAGCGGAGCTTC |
| | | AGACCTGGCGGGCTGTCTTTCGGGACGGCGCGCCCAAAGCGAGGGGC |
| | | CTTCTGCGCGAACTAGACTGTGCGCGCGGGGCGGCCGGCGAACTTAT |
| | | ACCAAGCTCGACCTCAGATCAGGCAGGAGTACCCGCTGAACTTAAGC |
| | | ATATCAATAAGCGGAGGAAAAGAAACCAACAGGGATTGCCCCAGTA |
| | | GCGGCGAGTGAAGCGGCAAAAGCTCAGATTTGGAATCGCTTCGGCGA |
| | | GTTGTGAATTGCAGGTTGGCGCCTCTGCGGCGGCGGCGGTCCAAGTC |
| | | CCTTGGAACAGGGCGCCATTGAGGGTGAGAGCCCCGTGGGACCGTTT |
| | | GCCTATGCTCTGAGGCCCTTCTGACGAGTCGAGTTGTTTGGGAATGCA |
| | | GCTCTAAGCGGGTGGTAAATTCCATCTAAGGCTAAATACTGGCGAGA |
| | | GACCGATAGCGAACAAGTACTGTGAAGGAAAGATGAAAAGCACTTTG |
| | | AAAAGAGAGTGAAACAGCACGTGAAATTGTTGAAAGGGAAGGGTAT |
| | | TGCGCCCGACATGGAGCGTGCGCACCGCTGCCCCTCGTGGGCGGCGC |
| | | TCTGGGCGTGCTCTGGGCCAGCATCGGTTTTTGCCGCGGGAGAAGGG |
| | | CGGCGGGCATGTAGCTCTTCGGAGTGTTATAGCCTGCCGCCGGCGCC |
| | | GCGAGCGGGGACCGAGGACTGCGACTTTTGTCTCGGATGCTGGCACA |
| | | ACGGCGCAACACCGCCCGTCTTGAAACATGGACCAAGGAGTCTAACG |
| | | TCTATGCGAGTGTTTGGGTGTGAAACCCCGGGCGCGTAATGAAAGTG |
| | | AACGTAGGTCGGACCGCTCCTCTCGGGGGGCGGGCACGATCGACCGA |
| | | TCCTGATGTCTTCGGATGGATTTGAGTAAGAGCATAGCTGTTGGGACC |
| | | CGAAAGATGGTGAACTATGCCTGAATAGGGTGAAGCCAGAGGAAACT |
| | | CTGGTGGAGGCTCGTAGCGGTTCTGACGTGCAAATCGATCGTCGAATT |
| | | TGGGTATAGGGGCGAAAGACTAATCGAACCATCTAGTAGCTGGTTCC |
| | | TGCCGAAGTTTCCCTCAGGA |
| 67 | DP53 Glutamine- | ATGAGCAAGCCCACTGTCGACCCCACTCTGAATCCAAAGGCTGGCCC |
| | tRNA ligase | TGCTGTCCCGGCTAACTTCCTGCGTCCAATCGTTCAGGCGGACCTAGA |
| | | CTCGGGTAAATACACACAGATCGTGACCCGCTTTCCGCCGGAGCCAA |
| | | ACGGCTATCTGCACATCGGTCATGCCAAATCCATTTGTGTGAACTTTG |
| | | GGCTGGCTCAAGAGTTTGGCGGCGTGACGCATTTGCGTTTTGACGACA |
| | | CCAACCCGGCAAAAGAAGACCAGGAATACATCGACGCCATCGAAAG |
| | | CGACGTCAAGTGGCTGGGCTTCGAGTGGGCCGGTGAAGTGCGTTACG |
| | | CGTCGCAATACTTCGATCAACTGCACGAGTGGGCGATTTACCTGATCA |
| | | AAGAAGGCAAGGCCTACGTCTGCGACCTGACGCCCGAGCAAGCCAAG |
| | | GAATACCGTGGCAGCCTGACCGAGCCCGGCAAGAACAGCCCGTTCCG |
| | | CGACCGTAGCGTTGAAGAGAACCTGGATCTGTTCGCCCGCATGACCG |
| | | CCGGTGAGTTTGAAGACGGCAAGCGTGTGCTGCGCGCCAAGATCGAC |
| | | ATGACCTCGCCGAACATGAACCTGCGCGACCCGATCATGTACCGCAT |
| | | CCGTCATGCCCATCACCACCAGACCGGTGACAAGTGGTGCATCTACC |
| | | CCAACTATGACTTCACCCACGGTCAGTCGGATGCCATTGAAGGCATC |
| | | ACCCATTCGATCTGCACCCTGGAGTTCGAAAGCCATCGTCCGCTGTAC |
| | | GAATGGTTCCTGGACAGCCTGCCAGTACCGGCGCGCCCGCGTCAGTA |
| | | CGAGTTCAGCCGTCTGAACCTCAACTACACCATCACCAGCAAGCGCA |
| | | AGCTCAAGCAGCTGGTCGATGAAAAGCACGTCAACGGCTGGGATGAC |
| | | CCGCGCATGTCGACGCTGTCGGGTTTCCGCCGTCGCGGTTACACGCCT |
| | | AAATCGATTCGTAATTTCTGTGACATGGTCGGCACCAACCGTTCTGAC |
| | | GGTGTTGTTGACTTCGGCATGCTGGAATTCAGCATTCGTGACGATTTG |
| | | GACCACAGCGCGCCGCGCGCCATGTGCGTGCTGCGTCCATTGAAGGT |
| | | GATTATTACCAACTACCCGGAAGGTCAGGTCGAAAACCTCGAGCTGC |
| | | CTTGCCACCCGAAAGAAGACATGGGTGTGCGGGTGTTGCCGTTTGCC |
| | | CGTGAAATCTACATCGACCGTGAAGACTTCATGGAAGAGCCGCCAAA |
| | | AGGCTACAAGCGTCTTGAGCCTGCGGGCGAAGTGCGTTTGCGCGGCA |
| | | GCTATGTGATCCGTGCCGACGAAGCGATCAAGGATGCCGATGGCAAC |
| | | ATCGTTGAACTGCATTGCTCGTACGATCCGCTGACCCTGGGTAAAAAC |
| | | CCTGAAGGTCGCAAGGTCAAGGGTGTTGTGCACTGGGTGCCGGCGGC |
| | | GGCCAGCGTCGAATGCGAAGTGCGTTTGTATGATCGTCTGTTCCGCTC |
| | | GCCGAACCCTGAAAAGGCCGAAGACGGCGCGGGCTTCCTGGAAAAC |
| | | ATCAACCCTGACTCGCTGCAGGTACTGACCGGTTGTCGTGCTGAACCC |
| | | TCGCTGGGCAATGCACAGCCGGAAGACCGTTTCCAGTTCGAGCGCGA |
| | | AGGCTACTTCTGCGCAGATATCAAGGACTCGAAACCCGGTCACCCGG |
| | | TATTCAACCGTACCGTGACCCTGCGTGATTCGTGGGGCCAGTGA |
| 68 | DP53 DNA | TTGAGCGAAGAAAACACGTACGACTCAACGAGCATTAAAGTGCTGAA |
| | gyrase subunit B | AGGCCTTGATGCCGTACGCAAACGTCCCGGTATGTACATTGGTGATAC |
| | | TGACGATGGCAGCGGTCTGCACCACATGGTGTTCGAAGTAGTCGACA |
| | | ACTCCATCGACGAAGCGCTGGCTGGCCATTGCGACGACATCACCATC |
| | | ACGATCCACCCGGACGAGTCCATCACCGTGCGCGATAACGGCCGCGG |
| | | TATTCCGGTTGACGTGCATAAAGAAGAAGGCGTATCTGCAGCCGAGG |
| | | TCATCATGACCGTGCTGCACGCCGGCGGTAAGTTCGATGACAACTCCT |
| | | ACAAAGTATCCGGCGGCTTGCACGGTGTAGGTGTTTCGGTGGTAAAC |
| | | GCCCTGTCCGAACTGCTGGTCTTGACTGTACGCCGCAGCGGCAAGATC |
| | | TGGGAACAGACCTACGTCCACGGTGTTCCTCAGGCGCCTATGGCTATT |
| | | GTGGGTGAAAGCGAAACCACGGGTACGCAGATCCACTTCAAGCCTTC |
| | | GGCTGAAACCTTCAAGAATATCCACTTTAGCTGGGACATCCTGGCCA |
| | | AGCGGATTCGTGAACTGTCCTTCCTGAACTCCGGTGTGGGTATCGTCC |
| | | TCAAGGACGAGCGCAGCGGCAAGGAGGAGCTGTTCAAGTACGAAGG |
| | | TGGCCTGCGTGCATTCGTTGATTACCTGAACACCAACAAGAACGCTGT |
| | | GAACCAGGTGTTCCACTTCAATGTTCAGCGTGAAGACGGCATCGGCG |
| | | TAGAAATCGCCCTGCAGTGGAACGACAGCTTCAACGAGAACCTGTTG |
| | | TGCTTCACCAACAACATTCCACAGCGCGATGGTGGCACGCACTTGGT |
| | | GGGCTTCCGCTCTGCCCTGACGCGTAACCTCAACACGTACATCGAAGC |
| | | TGAAGGCCTGGCCAAGAAGCACAAGGTCGCCACCACCGGTGATGACG |
| | | CCCGTGAAGGCTTGACCGCGATCATCTCGGTGAAAGTGCCGGATCCA |
| | | AAGTTCAGCTCGCAGACTAAAGACAAGCTGGTGTCTTCCGAAGTGAA |
| | | GACCGCTGTTGAACAGGAAATGGGCAAGTTCTTCTCCGACTTCCTGCT |
| | | GGAACACCCGAACGAAGCCAAGTTGATTGTCGGCAAGATGATCGACG |
| | | CAGCCCGTGCTCGTGAAGCTGCACGTAAAGCCCGTGAGATGACCCGT |
| | | CGTAAAGGCGCGTTGGACATCGCGGGCTTGCCGGGCAAGCTGGCTGA |
| | | CTGCCAGGAAAAAGACCCTGCTCTGTCCGAACTGTACCTGGTGGAAG |
| | | GTGACTCTGCTGGCGGCTCCGCCAAGCAGGGTCGCAACCGTCGTACC |
| | | CAAGCCATCCTGCCGTTGAAAGGTAAAATCCTCAACGTCGAGAAAGC |
| | | CCGTTTTGACAAGATGATCTCTTCGCAAGAAGTCGGCACCTTGATCAC |
| | | TGCGCTGGGCTGTGGCATCGGCCGCGAAGAGTACAACATCGACAAAC |
| | | TGCGCTATCACAACATCATCATCATGACCGATGCTGACGTTGACGGTT |
| | | CGCACATCCGTACCCTGCTGCTGACCTTCTTCTTCCGTCAGTTGCCGG |
| | | AGCTGATCGAGCGTGGCTACATCTACATCGCCCAGCCACCGTTGTACA |
| | | AAGTGAAAAAGGGCAAGCAAGAGCAGTACATCAAAGACGACGAGGC |
| | | CATGGAAGAGTACATGACCCAGTCGGCTCTTGAAGATGCCAGCCTGC |
| | | ACTTGAACGAAGATGCCCCTGGCATCTCCGGTGAGGCACTGGAGCGT |
| | | CTGGTGTACGACTTCCGCATGGTGATGAAGACCCTCAAGCGTTTGTCG |
| | | CGCCTGTACCCTCAGGAGCTGACCGAGCACTTCATCTACCTGCCGGCT |
| | | GTAAGCCTTGAGCAGTTGGGTGACCACGCTGCCATGCAGGACTGGAT |
| | | GGCCAAGTTTGAAGAGCGTCTGCGTCTGGTTGAGAAATCGGGCCTGG |
| | | TCTACAAAGCCAGCCTGCGTGAAGACCGTGAGCGTAATGTCTGGTTG |
| | | CCAGAGGTCGAACTGATCTCCCACGGCCACTCGACGTTCATCACCTTC |
| | | AACCGCGACTTCTTCGGCAGCAACGATTACAAAACCGTTGTGACCCT |
| | | GGGCGCTCAACTGAGCACCCTGCTGGATGAAGGCGCCTATATCCAGC |
| | | GTGGCGAACGTCGCAAGCAAGTGACCGAGTTCAAAGAAGCACTGGAC |
| | | TGGTTGATGGCTGAAAGCACCAAGCGTCACACCATCCAGCGCTACAA |
| | | AGGACTGGGTGAAATGAACCCGGATCAGCTCTGGGAAACCACGATGG |
| | | ACCCAAGCGTGCGTCGCATGCTGAAAGTCACCATCGAAGACGCGATC |
| | | GGCGCCGATCAGATCTTCAACACCTTGATGGGCGATGCTGTAGAACC |
| | | ACGTCGTGAATTCATCGAGAGCAACGCACTGGCAGTGTCCAACCTGG |
| | | ATTTCTGA |
| 69 | DP53 Isoleucine | ATGACCGACTACAAAGCCACGCTAAACCTCCCGGACACCGCCTTCCC |
| | tRNA ligase | AATGAAGGCCGGCCTGCCACAGCGCGAACCGCAAATTTTGCAGCGCT |
| | | GGGACAGCATTGGCCTGTACGGGAAGTTGCGCGAGATTGGCAAGGAT |
| | | CGTCCGAAGTTCGTACTTCACGACGGTCCTCCGTACGCCAACGGCACT |
| | | ATCCATATCGGTCATGCGCTGAACAAGATTCTGAAAGACATGATCAT |
| | | CCGCTCCAAGACCCTGTCGGGTTTTGACGCGCCGTATGTGCCGGGCTG |
| | | GGATTGCCATGGTTTGCCGATTGAACACAAGGTCGAAGTGACCCACG |
| | | GTAAAAACCTGAGCGCGGATAAAACCCGCGAGCTGTGCCGTGCCTAC |
| | | GCCACCGAGCAGATCGAGGGGCAGAAGTCCGAGTTCATCCGTCTGGG |
| | | TGTGCTGGGTGATTTCGCCAACCCGTACAAGACCATGGACTTCAAAA |
| | | ACGAAGCCGGTGAAATCCGTGCTTTGGCTGAGATCGTCAAGGGCGGT |
| | | TTTGTGTTCAAGGGCCTCAAGCCGGTGAACTGGTGCTTCGATTGCGGT |
| | | TCGGCCCTGGCTGAAGCTGAAGTTGAATACCAGGACAAGAAGTCTGC |
| | | GGCCATCGACGTTGCCTTCCCGGTTGCCGACGAGGCCAAGCTGGCCG |
| | | AGGCCTTTGGTCTGGCGGCACTGAGCAAACCTGCTTCGATCGTGATCT |
| | | GGACCACCACCCCGTGGACCATTCCGGCCAACCAGGCGCTTAACGTA |
| | | CACCCGGAATTCACCTACGCGCTGGTCGACGTGGGCGACAAGTTGCT |
| | | GGTACTGGCTGAAGAACTGGTCGAATCGAGTCTGGCGCGTTACAACC |
| | | TGCAGGGTTCGGTCATCGCCACCACCACTGGCTCAGCGCTTGAACTAA |
| | | TCAACTTCCGTCACCCGTTCTATGACCGTCTGTCGCCTGTTTATCTGGC |
| | | CGACTACGTTGAGCTGGGTGCTGGCACTGGTGTGGTTCACTCGGCTCC |
| | | AGCCTACGGCGTAGACGACTTCGTGACCTGCAAAGCCTATGGCATGG |
| | | TCAACGACGACATCATCAACCCGGTGCAAAGCAATGGCGTTTACGTG |
| | | CCGTCGCTGGAGTTCTTCGGTGGCCAGTTCATCTGGAAGGCCAACCAG |
| | | AACATCATCGACAAGCTGATCGAAGTCGGTTCGCTGATGTTCACCGA |
| | | GACCATCAGCCACAGCTATATGCACTGCTGGCGCCACAAGACGCCGC |
| | | TGATCTACCGTGCCACCGCCCAGTGGTTTATCGGTATGGACAAGCAGC |
| | | CGACTGATGGCGATACCTTGCGCACCCGTGCGCTGCAAGCGATCGAA |
| | | GACACCCAGTTCGTTCCGGCCTGGGGTCAGGCGCGCCTGCACTCGAT |
| | | GATCGCCAACCGCCCGGACTGGTGCATCTCGCGTCAACGCAACTGGG |
| | | GCGTGCCGATCCCGTTTTTCCTGAACAAGGAAAGCGGCGAGCTGCAC |
| | | CCGCGCACCGTCGAAATGATGGAAGAAGTGGCCAAGCGCGTTGAAGT |
| | | CGAAGGCATCGAGGCGTGGTTCAAGCTGGATGCTGCCGAGCTGCTGG |
| | | GCGACGAAGCGCCGCTGTACGACAAGATCAGCGATACCCTCGACGTC |
| | | TGGTTCGATTCGGGCACCACGCACTGGCATGTCCTTCGCGGTTCGCAC |
| | | CCGATGGGTCATGAAACCGGCCCACGCGCTGATCTCTACCTTGAAGG |
| | | CTCCGACCAGCACCGTGGCTGGTTCCACTCGTCGTTGCTGACCGGTTG |
| | | CGCCATCGACAACCACGCGCCGTACCGCGAGCTGCTGACCCACGGTT |
| | | TTACCGTGGACGAAGCGGGCCGCAAGATGTCCAAGTCGCTGGGCAAC |
| | | GTGATTGCACCGCAAAAGGTCAACGACACCCTGGGCGCCGACATCAT |
| | | GCGTCTGTGGGTTGCTTCGACCGACTACTCGGGCGAAATCGCGGTTTC |
| | | CGACCAGATCCTGCAGCGCAGTGCGGACGCCTACCGACGTATCCGCA |
| | | ATACCGCACGCTTCCTGCTGTCGAACCTGACCGGTTTCAATCCAGCCA |
| | | CCGACATCCTGCCTGCCGAAGAAATGCTGGCACTGGACCGCTGGGCG |
| | | GTGGATCGTGCGTTGCTGCTGCAACGTGAGCTGGAGCTGCATTACGG |
| | | CGAATACCGTTTCTGGAACGTGTACTCCAAGGTGCACAACTTCTGCGT |
| | | TCAGGAGCTGGGCGGTTTCTATCTCGACATCATCAAGGACCGCCAGT |
| | | ACACCACCGGCGCCAACAGCAAGGCTCGCCGTTCGTGCCAGACCGCG |
| | | CTGTTCCACATCTCTGAAGCGCTGGTGCGCTGGATCGCTCCGATCCTG |
| | | GCGTTCACCGCTGATGAGTTGTGGCAGTACCTGCCGGGCGAGCGCAA |
| | | CGAATCGGTCATGCTCAACACCTGGTACGAAGGCCTGACTGAACTGC |
| | | CGGAAGGCACCGAACTGGATCGCGCCTACTGGGAGCGAATCATGGCG |
| | | GTCAAGGTTGCGGTCAACAAGGAAATGGAAAACTTGCGCGCAGCCAA |
| | | GGCCATTGGCGGTAACTTGCAAGCAGAAGTGACCTTGTTCGCCGAAG |
| | | ATCAGCTGGCTGCTGATTTGTCCAAGTTGAGCAACGAACTGCGTTTCG |
| | | TGTTGATCACCTCCACTGCCAGCGTTGCGCCTTTTGCGCAGGCTCCAG |
| | | CAGATGCCGTGGTTACCGAAGTGGCTGGCCTCAAACTCAAGGTGGTC |
| | | AAGTCGGCCCATGCCAAGTGCGCCCGTTGCTGGCACTGCCGTGAAGA |
| | | CGTCGGCGTTAACCCCGAGCACCCTGAAATCTGCGGTCGTTGTGTAGA |
| | | CAATATCAGCGGCGCTGGTGAGGTACGTCACTATGCCTAA |
| 70 | DP53 NADH- | ATGACTGCAGGCTCCGCTCTGTACATCCCGCCTTACAAGGCTGACGAC |
| | quinone | CAAGATGTGGTTGTCGAACTCAATACCCGTTTTGGCCCTGAGGCGTTC |
| | oxidoreductase | ACCGCCCAGGCCACGCGCACCGGCATGCCGGTGCTTTGGGTTAGCCG |
| | subunit C/D | CGCAAAACTGGTCGAAGTACTGACCTTCCTGCGCAACCTGCCAAAAC |
| | | CCTACGTCATGCTCTATGACCTGCACGGTGTGGACGAACGTCTGCGTA |
| | | CCAAGCGTCAGGGCCTGCCATCGGGTGCAGACTTCACCGTCTTCTACC |
| | | ACCTGATGTCGCTGGAACGTAACAGCGACGTCATGATCAAGGTGGCC |
| | | CTGTCTGAAAAAGACCTGAGTGTCCCTACCGTGACCGGTATCTGGCCG |
| | | AACGCCAACTGGTACGAGCGTGAAGTCTGGGACATGTTCGGCATCGA |
| | | TTTCAAAGGCCACCCGCACCTGTCGCGCATCATGATGCCGCCGACCTG |
| | | GGAAGGTCACCCGCTGCGCAAGGACTTCCCGGCCCGTGCCACAGAGT |
| | | TCGATCCGTACAGCCTGACCCTGGCCAAGGTGCAGCTGGAAGAGGAA |
| | | GCCGCGCGCTTCCGCCCGGAAGACTGGGGCATGAAACGCTCCGGTGA |
| | | AAACGAGGACTACATGTTCCTCAACCTGGGCCCTAACCACCCTTCGGC |
| | | TCACGGTGCCTTCCGCATCATCCTGCAGCTGGACGGTGAAGAGATCGT |
| | | CGACTGCGTGCCTGACGTCGGTTACCACCACCGTGGCGCCGAGAAAA |
| | | TGGCCGAACGCCAGTCCTGGCACAGTTTCATCCCGTACACCGACCGG |
| | | ATCGATTACCTCGGCGGAGTGATGAACAACCTGCCGTACGTGCTCTCG |
| | | GTCGAGAAGCTGGCCGGTATCAAAGTGCCGGATCGGGTCGACACCAT |
| | | CCGCATCATGATGGCCGAATTCTTCCGTATCACCAGCCACCTGCTGTT |
| | | CCTGGGTACCTATATCCAGGACGTGGGCGCCATGACCCCGGTGTTCTT |
| | | CACGTTCACCGACCGTCAGCGCGCTTACAAGGTGATCGAGGCCATCA |
| | | CCGGTTTCCGTCTGCACCCGGCCTGGTACCGCATCGGCGGCGTTGCCC |
| | | ACGACCTGCCGAACGGCTGGGATCGCCTGGTCAAGGAATTCATCGAC |
| | | TGGATGCCCAAGCGTCTGGACGAGTACCAGAAAGCCGCTCTGGACAA |
| | | CAGCATCCTGCGTGGTCGTACCATCGGCGTTGCCGCCTACAACACCAA |
| | | AGAGGCCCTGGAATGGGGCGTCACCGGTGCCGGCCTGCGCTCCACCG |
| | | GTTGTGACTTCGATATCCGCAAGGCGCGCCCGTATTCCGGCTACGAGA |
| | | ACTTCGAATTCGAAGTCCCGCTGGCAGCCAACGGCGATGCCTACGAT |
| | | CGTTGCATCGTGCGCGTCGAAGAAATGCGCCAGAGCCTGAAAATCAT |
| | | CGAGCAGTGCATGCGCAACATGCCGGCCGGCCCGTACAAGGCGGATC |
| | | ACCCGCTGACCACGCCGCCGCCTAAAGAACGCACGCTGCAGCATATC |
| | | GAGACCTTGATCACGCACTTCCTGCAAGTTTCGTGGGGCCCGGTGATG |
| | | CCGGCCAACGAATCCTTCCAGATGATCGAAGCGACCAAGGGCATCAA |
| | | CAGTTATTACCTGACGAGCGATGGCGGCACCATGAGCTACCGCACCC |
| | | GGATTCGCACCCCAAGCTTCCCGCACCTGCAACAGATCCCTTCGGTGA |
| | | TCAAAGGTGAAATGGTCGCGGACTTGATTGCGTACCTGGGTAGTATC |
| | | GATTTCGTTATGGCCGACGTGGACCGCTAA |
| 71 | DP53 Protein | ATGGACGACAACAAGAAGAAAGCCTTGGCTGCGGCCCTGGGTCAGAT |
| | RecA | CGAACGTCAATTCGGCAAGGGTGCCGTGATGCTGATGGGCGACCAGG |
| | | AGCGTCAGGCAGTCCCGGCGATCTCCACCGGCTCCCTGGGTCTGGAC |
| | | ATCGCACTGGGCATTGGCGGTCTGCCAAAAGGCCGTATTGTTGAAAT |
| | | CTACGGCCCTGAGTCGTCGGGTAAAACCACACTGACCCTGTCCGTGAT |
| | | TGCCCAGGCGCAAAAGGCCGGTGCTACCTGCGCCTTCGTCGATGCCG |
| | | AGCACGCCCTTGATCCTGAGTACGCTGCCAAACTGGGCGTAAACGTT |
| | | GATGACCTGCTGGTTTCACAGCCTGACACCGGCGAACAGGCACTGGA |
| | | AATCACCGATATGCTGGTGCGTTCCAATGCGGTTGACGTGATCATCAT |
| | | CGACTCCGTTGCTGCACTGACGCCAAAAGCTGAAATCGAAGGCGACA |
| | | TGGGCGATACCCACGTTGGCCTGCAAGCCCGTCTGATGTCGCAAGCG |
| | | CTGCGTAAAATCACCGGTAACATCAAGAACGCCAACTGCCTGGTTAT |
| | | CTTCATCAACCAGATCCGCATGAAAATCGGCGTGATGTTCGGCAGCC |
| | | CTGAAACCACCACCGGTGGTAACGCACTGAAGTTCTACGCTTCGGTA |
| | | CGTCTGGATATCCGCCGCACCGGCGCCGTAAAAGAAGGCGATGTGGT |
| | | GGTGGGTAGCGAAACCCGCGTGAAAGTGGTCAAGAACAAGGTGGCA |
| | | CCACCGTTCCGTCAGGCTGAATTCCAGATCCTGTACGGCAAGGGTATC |
| | | TACCTGAACGGTGAAATGATTGACCTGGGCGTACTGCATGGCTTTGTT |
| | | GAAAAAGCTGGCGCCTGGTACAGCTACAACGGCAGCAAAATCGGTCA |
| | | GGGCAAGGCCAACTCCGCCAAGTTCCTGGACGATAACCCGGACATCA |
| | | AGGATGCGCTGGAGAAGCAGCTGCGTGAGAAGTTGCTCGGGCCAAAA |
| | | ACCGATGCCGAACTGGCAGCGACGGACTGCAATGGACCTGCTCGCGC |
| | | GACGCGAGCACGGTCGAGTCGAGCTGACGCGCAAGTTGCGTCAGCGC |
| | | GGCGCTTGCCCCGACATGATCGACGCTGCCCTTGA |
| 72 | DP53 RNA | ATGTCCGGAAAAGCGCAACAGCAGTCTCGTATCAAAGAGTTGATCAC |
| | polymerase sigma | CCTCGGCCGTGAGCAGAAGTATCTGACTTACGCAGAGGTCAACGACC |
| | factor RpoD | ACCTGCCCGAAGATATTTCAGATCCGGAGCAAGTGGAAGACATCATC |
| | | CGCATGATTAATGACATGGGGATCCCCGTACACGAGAGTGCTCCGGA |
| | | TGCGGACGCCCTTATGTTGGCCGATGCCGACACCGACGAAGCAGCAG |
| | | CTGAAGAAGCGGCTGCAGCGTTGGCGGCAGTAGAGACCGACATTGGT |
| | | CGTACTACCGACCCTGTGCGCATGTATATGCGTGAAATGGGCACGGT |
| | | AGAACTGCTGACACGTGAAGGCGAAATCGAAATCGCCAAGCGTATCG |
| | | AAGAAGGCATCCGTGAAGTGATGGGCGCAATCGCGCACTTCCCTGGC |
| | | ACGGTTGACCATATTCTCTCCGAGTACACTCGCGTCACCACCGAAGGT |
| | | GGCCGCCTGTCCGACGTTCTGAGCGGTTATATCGACCCGGACGACGG |
| | | TATTGCGCCGCCCGCAGCCGAAGTACCTCCTCCTGTCGACACCAAGGT |
| | | GAAAGCCGAAGGTGATGACGAAGAGGACGACAAGGAAGATTCCGGC |
| | | GAAGACGAGGAAGAGGTCGAAAGCGGCCCTGATCCGATCATCGCGG |
| | | CCCAGCGCTTTGGCGCTGTTTTCGATCAGATGGAAATCGCTCGCAAGG |
| | | CCCTGAAAAAGCACGGTCGCGGCAGCAAGCAGGCAATTGCCGAGCTG |
| | | GTTGCACTGGCTGAGCTGTTCATGCCGATCAAACTGGTTCCGAAGCAA |
| | | TTCGAAGGCCTGGTTGAGCGTGTTCGCAGCGCCCTGGAGCGTCTGCGT |
| | | GCACAAGAGCGCGCAATCATGCAGCTGTGTGTACGTGATGCACGCAT |
| | | GCCGCGCACCGATTTCCTGCGTCTGTTCCCGGGCAACGAAGTCGACG |
| | | AAAGCTGGAGCGATGCGCTGGCCAAAGGCAAAAGCAAATATGCTGA |
| | | AGCCATTGGTCGCCTGCAACCGGACATCATCCGTTGCCAGCAAAAGC |
| | | TCTCTGCTCTGGAAGCAGAAACCGGCTTGAAGATTGCCGAGATCAAG |
| | | GACATCAACCGTCGCATGTCGATCGGCGAGGCCAAGGCCCGCCGCGC |
| | | GAAGAAAGAAATGGTTGAAGCCAACTTGCGTCTGGTGATCTCCATCG |
| | | CCAAGAAGTACACCAACCGTGGCCTGCAGTTCCTCGATCTGATCCAG |
| | | GAAGGCAACATCGGCTTGATGAAAGCGGTAGACAAGTTTGAATACCG |
| | | CCGCGGCTACAAATTCTCGACTTATGCCACCTGGTGGATCCGTCAGGC |
| | | GATCACTCGCTCGATCGCCGACCAGGCCCGCACCATCCGTATTCCGGT |
| | | GCACATGATCGAGACGATCAACAAGCTCAACCGTATTTCCCGTCAGA |
| | | TGTTGCAGGAAATGGGCCGTGAACCGACCCCGGAAGAGCTGGGCGAA |
| | | CGCATGGAAATGCCTGAGGATAAAATCCGCAAGGTATTGAAGATCGC |
| | | TAAAGAGCCGATCTCCATGGAAACCCCGATCGGTGATGACGAAGACT |
| | | CCCATCTGGGTGACTTCATCGAAGACTCGACCATGCAGTCGCCAATCG |
| | | ATGTTGCTACCGTTGAGAGCCTTAAAGAAGCGACACGCGACGTACTC |
| | | GGCGGCCTCACAGCCCGTGAAGCCAAGGTACTGCGCATGCGTTTCGG |
| | | TATCGACATGAATACCGACCACACCCTTGAGGAGGTTGGTAAACAGT |
| | | TCGACGTTACCCGTGAGCGGATTCGTCAGATCGAAGCCAAGGCGCTG |
| | | CGCAAGCTGCGCCACCCGACGAGAAGCGAGCATTTGCGCTCCTTCCT |
| | | CGACGAGTGA |
| 73 | DP53 DNA- | ATGGCTTACTCATATACTGAGAAAAAACGTATCCGCAAGGACTTTAG |
| | directed RNA | CAAGTTGCCGGACGTCATGGATGTGCCGTATCTCTTGGCAATCCAGCT |
| | polymerase | GGATTCGTATCGTGAATTCTTGCAGGCGGGAGCGACTAAAGATCAGT |
| | subunit beta | TCCGCGACGTGGGCCTGCATGCGGCCTTCAAATCCGTTTTCCCGATCA |
| | | TCAGCTACTCCGGCAATGCTGCGCTGGAGTACGTCGGTTATCGCTTGG |
| | | GCGAACCGGCATTTGATGTCAAAGAATGCGTGTTGCGTGGCGTAACG |
| | | TACGCCGTACCTTTGCGGGTAAAAGTTCGTTTGATCATTTTCGACAAA |
| | | GAATCGTCGAACAAAGCGATCAAGGACATCAAAGAGCAAGAAGTCT |
| | | ACATGGGTGAAATCCCCCTGATGACTGAAAACGGTACCTTCGTAATC |
| | | AACGGTACCGAGCGTGTAATTGTTTCCCAGCTGCACCGTTCCCCGGGC |
| | | GTGTTCTTTGCCACGACCGCGGCAAGACGCACAGCTCCGGTAAGCTG |
| | | CTTTATTCCGCGCGTATCATTCCTTACCGTGGTTCGTGGCTCGACTTCG |
| | | AGTTCGACCCGAAAGACTGCGTGTTCGTGCGTATTGACCGTCGTCGCA |
| | | AGCTGCCTGCATCGGTATTGCTGCGCGCGCTGGGTTATACCACTGAGC |
| | | AAGTGCTGGACGCGTTCTACACCACCAACGTGTTCCACGTTCAGGGTG |
| | | AGAGCATCAGCCTGGAGCTGGTTCCACAGCGTCTGCGCGGTGAAATC |
| | | GCGGCCATCGACATTACCGATGACAAAGGCAAGGTGATTGTTGAGCA |
| | | GGGTCGTCGTATCACTGCTCGTCATATCAACCAGCTGGAAAAAGCCG |
| | | GTGTCAAAGAGCTCGTTATGCCTCTGGACTATGTCCTGGGTCGCACAA |
| | | CGGCCAAGGCTATCGTGCATCCGGCTACTGGCGAAATCATTGCTGAG |
| | | TGCAACACCGAGCTGACCACTGAAATCCTGGCAAAAGTTGCCAAGGG |
| | | CCAGGTTGTTCGCATCGAAACGTTGTACACCAACGATATCGACTGCG |
| | | GTCCGTTCGTCTCCGACACGCTGAAGATCGACTCCACCAGCAACCAA |
| | | CTGGAAGCGCTGGTCGAAATCTATCGCATGATGCGTCCAGGCGAGCC |
| | | GCCAACCAAAGACGCTGCCGAGACTCTGTTCAACAACCTGTTCTTCAG |
| | | CCCTGAGCGCTATGACCTGTCTGCGGTCGGCCGGATGAAGTTCAACC |
| | | GTCGTATCGGTCGTACCGAGATCGAAGGTTCGGGCGTGTTGTGCAAA |
| | | GAAGACATCGTTGCCGTGCTGAAGACCCTGGTCGACATCCGTAACGG |
| | | TAAAGGCATCGTCGATGACATCGACCACCTGGGTAACCGTCGTGTTC |
| | | GCTGTGTAGGCGAAATGGCCGAGAACCAGTTCCGCGTTGGCCTGGTA |
| | | CGTGTTGAGCGTGCGGTCAAAGAGCGTCTGTCGATGGCTGAAAGCGA |
| | | AGGCCTGATGCCGCAAGACCTGATCAACGCCAAGCCTGTGGCTGCGG |
| | | CGGTGAAAGAGTTCTTCGGTTCCAGCCAGCTGTCCCAGTTCATGGACC |
| | | AGAACAACCCTCTGTCCGAGATCACCCACAAGCGCCGTGTTTCTGCAC |
| | | TGGGCCCGGGCGGTCTGACGCGTGAGCGTGCGGGCTTTGAAGTTCGT |
| | | GACGTACACCCGACTCACTACGGCCGTGTTTGCCCTATTGAGACGCCG |
| | | GAAGGTCCGAACATCGGTCTGATCAACTCCCTGGCTGCCTATGCGCGC |
| | | ACCAACCAGTACGGCTTCCTCGAGAGCCCGTACCGTGTAGTGAAAGA |
| | | CGCACTGGTAACTGACGAGATCGTTTTCCTGTCCGCCATCGAAGAAGC |
| | | TGATCACGTGATCGCTCAGGCCTCGGCCACGATGAACGACAAGAAAG |
| | | TGCTGATCGACGAGCTGGTTGCTGTTCGTCACTTGAACGAATTCACCG |
| | | TCAAGGCGCCGGAAGACGTCACCTTGATGGACGTTTCGCCGAAGCAG |
| | | GTTGTTTCGGTTGCAGCGTCGCTGATCCCGTTCCTGGAACACGATGAC |
| | | GCCAACCGTGCGTTGATGGGTTCCAACATGCAGCGTCAAGCTGTACC |
| | | AACCCTGCGCGCTGACAAGCCGCTGGTAGGTACCGGCATGGAGCGTA |
| | | ACGTAGCTCGTGACTCCGGCGTTTGCGTCGTGGCTCGTCGTGGCGGCG |
| | | TGATCGACTCTGTTGATGCCAGCCGTATCGTGGTTCGTGTTGCTGATG |
| | | ACGAAGTTGAAACTGGCGAAGCCGGTGTCGACATCTACAACCTGACC |
| | | AAATACACCCGTTCCAACCAGAACACTTGCATCAACCAGCGTCCGCT |
| | | GGTGCGCAAGGGTGACCGTGTACAGCGTAGCGACATCATGGCTGACG |
| | | GCCCGTCCACCGATATGGGTGAACTGGCGCTGGGTCAAAACATGCGC |
| | | ATCGCGTTCATGGCCTGGAACGGTTACAACTTCGAAGACTCCATCTGC |
| | | TTGTCGGAACGAGTTGTTCAAGAAGACCGCTTTACCACGATCCACATT |
| | | CAGGAACTGACCTGTGTGGCACGTGACACCAAGCTTGGGCCTGAAGA |
| | | GATCACTGCAGACATCCCTAACGTGGGTGAAGCTGCACTGAACAAAC |
| | | TGGACGAAGCCGGTATCGTTTACGTAGGTGCTGAAGTTGGCGCCGGC |
| | | GACATTCTGGTAGGTAAGGTCACTCCGAAAGGCGAGACCCAGCTGAC |
| | | TCCGGAAGAGAAGCTGTTGCGTGCCATCTTCGGTGAAAAAGCCAGCG |
| | | ACGTTAAAGACACCTCCCTGCGCGTACCTACCGGTACCAAAGGTACT |
| | | GTTATCGACGTGCAGGTCTTCACCCGTGACGGCGTTGAGCGTGATGCT |
| | | CGTGCACTGTCGATCGAGAAGACCCAGCTGGACGAGATCCGCAAGGA |
| | | TCTGAACGAAGAGTTCCGTATCGTTGAAGGCGCTACCTTCGAACGTCT |
| | | GCGCTCTGCTCTGGTTGGCCGCATTGCCGAAGGTGGTGCCGGTCTGAA |
| | | GAAAGGTCAGGAAATCACCAATGAAATCCTGGACGGTCTTGAGCATG |
| | | GTCAGTGGTTCAAACTGCGCATGGCTGAAGATGCTCTGAACGAGCAG |
| | | CTTGAAAAGGCTCAGGCTTACATCATCGATCGCCGTCGTCTGCTGGAC |
| | | GACAAGTTCGAAGACAAGAAGCGCAAACTGCAGCAGGGCGATGACC |
| | | TGGCTCCAGGCGTGCTGAAAATCGTCAAGGTTTACCTGGCAATCCGCC |
| | | GTCGCATCCAGCCGGGTGACAAGATGGCCGGTCGTCACGGTAACAAG |
| | | GGTGTGGTCTCCGTGATCATGCCGGTTGAAGACATGCCGTACGATGCC |
| | | AATGGCACCCCGGTTGATGTGGTCCTCAACCCGTTGGGCGTACCTTCG |
| | | CGTATGAACGTTGGTCAGATTCTCGAAACTCACCTGGGCCTCGCGGCC |
| | | AAAGGTCTGGGCGAGAAGATCAACCTCATGATTGAAGAACAACGCAA |
| | | GGTCGCTGACCTGCGTAAGTTCCTGCATGAGATCTACAACGAAATTG |
| | | GCGGTCGTCAAGAAAGCCTGGATGACTTCTCCGATCAGGAAATCCTG |
| | | GATCTGGCGAAGAACCTTCGCGGCGGTGTGCCAATGGCTACCCCGGT |
| | | GTTCGACGGTGCCAAGGAAAGCGAAATCAAGGCAATGCTTCGTTTGG |
| | | CAGACCTGCCAGACAGCGGCCAGATGGTGCTGACTGATGGTCGTACC |
| | | GGCAACAAGTTCGAGCGTCCGGTTACCGTTGGCTACATGTACATGCTG |
| | | AAGCTGAACCACTTGGTAGACGACAAGATGCACGCTCGTTCTACCGG |
| | | TTCTTACAGCCTGGTTACCCAGCAGCCGCTGGGTGGTAAGGCGCAGTT |
| | | CGGTGGTCAGCGTTTCGGGGAGATGGAGGTCTGGGCGCTGGAAGCCT |
| | | ACGGCGCGGCATACACTCTGCAAGAAATGCTCACAGTGAAGTCGGAC |
| | | GATGTGAACGGCCGTACCAAGATGTACAAAAACATCGTGGACGGCGA |
| | | TCACCGTATGGAGCCGGGCATGCCCGAGTCCTTCAACGTGTTGATCAA |
| | | AGAAATTCGTTCCCTCGGCATCGATATCGATCTGGAAACCGAATAA |
| 74 | DP9 Glycine | ATGGCACATAATTATTTACTAGAAATTGGATTGGAAGAAATTCCGGC |
| | tRNA ligase beta | CCATGTTGTAACTCCAAGTATCAAACAGTTAGTACAAAAAGTAACAG |
| | subunit | CCTTCTTAAAAGAAAATCGCTTAACATACGACTCAATTGATCATTTTT |
| | | CAACTCCTCGTCGTTTGGCAATTCGAATCAATGGGTTAGGCGACCAAC |
| | | AACCTGATATTGAAGAAGATGCTAAAGGCCCTGCTCGTAAAATTGCT |
| | | CAAGATGCTGATGGAAATTGGACTAAGGCTGCAATTGGCTTTACACG |
| | | TGGACAAGGTCTTACGGTTGACGATATTACTTTTAAAACAATCAAAG |
| | | GTACGGACTATGTGTACGTCCATAAGTTAATCAAAGGAAAGATGACT |
| | | AAGGAAATCCTTACGGGGATAAAAGAAGTTGTTGAATCAATTAATTT |
| | | CCCAACAATGATGAAGTGGGCTAACTTTGATTTTAAATATGTACGCCC |
| | | AATTCGTTGGCTGGTTTCTATTCTAGATGAAGAAGTCCTTCCTTTTAGT |
| | | ATCTTAGACGTAACTGCGGGACGCCGAACAGAAGGACATCGTTTCTT |
| | | AGGTGAAGCTGTCGAACTGGCTAATGCTGAAGAATATGAAGCAAAAT |
| | | TACACGATCAATTTGTGATTGTTGATGCCGACGAGCGTAAACAATTAA |
| | | TTTCAAACCAAATTAAAGCAATTGCTGAAAGCAATCGTTGGAACGTT |
| | | ACCCCTAACCCAGGTCTTTTAGAAGAGGTTAACAATTTGGTTGAGTGG |
| | | CCAACCGCTTTTAATGGGGGATTTGATGAAAAGTATTTAGCTATTCCA |
| | | GAAGAGGTATTGATAACATCAATGCGTGACCACCAACGCTTCTTCTTT |
| | | GTCCGCGACCAAGCTGGAAAGCTATTGCCAAACTTCATCTCCGTACG |
| | | AAATGGGAATGAAGAATTTATTGAAAATGTTGTTCGTGGAAATGAAA |
| | | AAGTTTTAACTGCACGTTTAGAAGACGCTGCTTTCTTCTACGAAGAAG |
| | | ATCAAAAACATGATATTAATTATTATGTTGACCGACTTAAAAAGGTTA |
| | | GTTTCCATGATAAGATTGGTTCAATGTACGAAAAAATGCAACGAGTT |
| | | AATTCTATTGCTAAAGTTATTGGAAACACCTTAAATCTTAATCAAACG |
| | | GAACTTGATGATATCGATCGCGCTACAATGATTTATAAATTTGATTTG |
| | | GTAACTGGTATGGTTGGTGAGTTCTCAGAATTACAAGGAGTAATGGG |
| | | TGAAAAATATGCTCAACTTAATGGTGAAAACCAAGCAGTAGCCCAAG |
| | | CCATTCGCGAACATTACATGCCAAATAGCGCAGAAGGTGATTTGCCT |
| | | GAAAGTGTAACGGGCGCGGTAGTCGCATTAGCTGATAAGTTTGATAA |
| | | CATCTTTAGTTTTTTCTCAGCTGGTATGATTCCAAGTGGTTCAAACGAT |
| | | CCATATGCATTACGCCGACATGCATATGGAATTGTTAGAATCTTAAAT |
| | | AGCCGTGATTGGCAATTAGATTTAAATCAATTCAAATCACAATTTAAG |
| | | ACTGAATTAGCGGAGAATGGCACAGCGTTTGGTGTGGATGTCGATCA |
| | | AAACTTTGACCAAGTACTTAACTTCTTTAATGACCGTATTAAACAATT |
| | | GCTTGATCATCAAAAGATTAGTCATGATATCGTTGAAACGGTGCTTAC |
| | | AGGTAATAATCATGATGTTACGGAAATTATCGAAGCTGCCCAAGTAC |
| | | TAGCAGATGCTAAAGCGAGCTCTACATTTAAAGATGATATTGAAGCT |
| | | TTAACACGAGTTCAAAGAATTGCTACAAAGAATGAAGAAAGTGGAGA |
| | | ACTTAATGTAGATCCACAATTATTTAATAATGCTTCTGAAGGCGAACT |
| | | TTTTGATCAAATTATTAAAATTGAAGCTGCAAATAATTTGACAATGAG |
| | | CCAACTATTTGCTAAATTATGCGAGTTGACTCCTGCGATTAGCAAGTA |
| | | CTTTGACGCAACGATGGTCATGGACAAAGACGAAAATATTAAGTGTA |
| | | ATCGTTTGAATATGATGAGTCGGTTAGCTAATTTAATTCTAAAAATTG |
| | | GGGATCTAACTAACGTACTTGTAAAATAA |
| 75 | DP9 Glutamine | ATGGCAAAGAAAAATTATTCGCAAGCAGATATTCGTCAGATGGCAAA |
| | synthetase | GGATGAAAATGTACGTTTTCTCCGATTAATGTTTACAGATCTTTTTGG |
| | | AATAATTAAGAACGTTGAAGTACCAATTAGTCAATTGGACAAACTAT |
| | | TAGATAATAAATTGATGTTTGATGGTTCCTCAATTGACGGGTTTGTTC |
| | | GGATTGAAGAAAGTGACATGTATTTATACCCAGATCTTTCTACTTGGA |
| | | TGGTTTTCCCATGGGGAAGCGAACATGGCAAGGTGGCTCGCATTATTT |
| | | GTGAAGTATACTCAAATGATCGTAAACCATTCGTGGGTGATCCACGT |
| | | AACAATTTAATTCGAGTACTCCAAGAGATGAAGGATGCAGGATTTAC |
| | | TGATTTTAATATCGGACCTGAACCTGAGTTTTTCTTGTTGAAATTAGA |
| | | TGAAAATGGTAAACCAACCACTAATTTAAATGATAAAGGTAGTTACT |
| | | TTGATTTAGCTCCTGTTGATTTAGGTGAAAACTGCCGTCGTGATATTG |
| | | TTTTGGAACTTGAAAATATGGGCTTTGATGTTGAAGCTTCTCATCATG |
| | | AAGTTGCTCCAGGACAACACGAAATTGACTTTAAATACGCCGATGCT |
| | | TTGACCGCTGCCGATAACATTCAAACCTTTAAGTTGGTTGTTAAGACA |
| | | GTTGCCCGTAAATATAACCTGCATGCTACATTTATGCCTAAACCTATG |
| | | GATGGAATCAATGGTTCAGGGATGCATTTAAACATGTCACTTTTCAAT |
| | | AAGGAAGGCAATGCTTTCTATGACGAAAAGGGTGACTTACAACTTTC |
| | | TCAAAATGCTTACTGGTTCCTTGGTGGACTATTGAAGCATGCTCGTAG |
| | | TTATACGGCCGTATGTAACCCAATTGTTAACTCGTACAAACGTTTAGT |
| | | TCCTGGATATGAAGCTCCAGTATACGTTGCTTGGTCAGGTTCAAATCG |
| | | TTCACCACTTATTCGCGTTCCTTCAAGTAAGGGACTCTCAACTCGTTTT |
| | | GAAGTTCGAAGCGTCGATCCAGCTGCTAACCCATACTTAGCAATTGC |
| | | ATCAGTATTGGAAGCAGGCTTAGATGGCATTAGAAACAAGATTGAAC |
| | | CAGAAGATTCCGTTGATCGTAATATCTATCGAATGAACATTCAAGAA |
| | | CGTAATGAAGAGCATATTACAGATCTACCTTCAACATTACACAATGCT |
| | | TTGAAGGAATTCCAAAATGATGATGTAATGCGTAAGGCATTAGGAGA |
| | | TCACATTTTCCAAAGCTTCCTCGAAGCTAAGAAGTTAGAATGGGCTTC |
| | | TTACCGTCAAGAAGTGACACAATGGGAACGTGATCAATATCTCGAAA |
| | | TGTTCTAG |
| 76 | DP9 DNA gyrase | TTGGCAGACGAAAAAGAAACGAAAGCAGAATTAGCCAGAGAATATG |
| | subunit B | ATGCGAGTCAAATTCAGGTTTTAGAGGGGCTCGAAGCAGTTCGTAAA |
| | | CGCCCAGGAATGTATATTGGGTCGACTAGTTCTCAAGGACTACACCAT |
| | | TTGGTTTGGGAAATTATTGATAATGGTATTGATGAAGCTCTTGCAGGA |
| | | TTTGCAGACAAAATTGATGTGATCGTTGAAAAAGACAATAGTATTAC |
| | | CGTCACTGATAATGGACGTGGGATTCCGGTTGATATCCAAAAGAAAA |
| | | CTGGAAAACCAGCTTTAGAAACAGTCTTTACGGTCCTACATGCCGGA |
| | | GGTAAATTCGGCGGTGGCGGTTATAAAGTTTCTGGAGGATTGCATGG |
| | | TGTGGGCGCATCCGTTGTAAATGCGTTATCAACGGAATTAGATGCGC |
| | | GCGTCATGAAGGACGGTAAAATCTATTACATTGATTTTGCGCTAGGA |
| | | AAAGTAAAAACACCGATGAAAACGATTGGTGATACTGAACATCCTGA |
| | | CGATCATGGAACTATTGTTCATTTCGTTCCAGATCCAGATATTTTCCA |
| | | AGAAACTACCACATACGACATTAATATCTTAAAAACACGAATTCGTG |
| | | AATTAGCCTTTTTGAACAAAGGTCTACGGATTACTTTGAAGGATATGC |
| | | GTCCTGAAAAGCCAACTGAAGACGACTTCTTGTATGAAGGTGGGATT |
| | | CGCCACTACGTTGAATATCTAAACGAAGGCAAAGAAGTAATTTTCCC |
| | | TGAACCTATCTATGTTGAAGGGGTTACAAAAGGTATCACTGTTGAAGT |
| | | AGCTATGCAATATATCGAAGGTTATCAAAGTAAATTGTTAACTTTTAC |
| | | TAACAATATTCATACTTACGAAGGCGGTACCCACGAAGAAGGTTTCA |
| | | AACGTGCTTTAACACGAGTTATTAACGATTACGCTAAAAACAACAAT |
| | | ATTTTAAAAGAAAATGATGATAAATTGTCTGGTGATGATGTTCGAGA |
| | | AGGTTTGACGGCAGTAGTCAGCGTTAAGCATCCTGATCCTCAATTCGA |
| | | AGGACAAACGAAAACAAAATTGGGTAACTCAGATGCTCGGACAGCTG |
| | | TTAACGAAGTGTTTGCTGAAACTTTCAATAAATTCTTATTGGAAAATC |
| | | CTAAGGTTGCACGTCAAATTGTTGATAAGGGAATCTTGGCAGCAAAA |
| | | GCAAGAGTCGCCGCTAAACGAGCTCGTGAAGTTACGCGTAAGAAGAG |
| | | TGGCCTAGAACTCAATAATCTTCCTGGTAAATTAGCTGATAATACTTC |
| | | TAAGGATCCTTCAATTAGTGAATTATTCATTGTCGAGGGTGATTCTGC |
| | | CGGTGGTAGTGCTAAGTCGGGACGTTCGCGTCTCACACAAGCTATTTT |
| | | GCCAATTCGTGGGAAGATTTTGAACGTTGAAAAAGCCACTTTGGATC |
| | | GGGTTTTGGCCAATGAAGAAATTCGTTCACTCTTTACAGCGCTCGGAA |
| | | CTGGATTTGGTGAGGACTTTGATGTAAGTAAAGCCAACTATCATAAAT |
| | | TGATTATCATGACCGATGCCGATGTCGATGGTGCTCATATTCGGACAC |
| | | TATTATTGACGCTGTTCTATCGTTACATGCGTCCAATGATTGATGCAG |
| | | GATTTGTTTACATTGCTCAACCACCGCTCTACCAAGTACGTCAAGGTA |
| | | AGATGATTCAATATATCGATTCTGATGAAGAATTAGAAACAGTACTT |
| | | GGACAATTGTCACCATCACCAAAACCTGTAATTCAACGTTATAAAGG |
| | | TCTTGGTGAAATGGATGCTGAGCAACTTTGGGAAACAACCATGAATC |
| | | CAGAAAATCGACGCTTGTTACGAGTTTCAGCCGAAGATGCTGATGCT |
| | | GCAAGTGGTGATTTTGAAATGTTGATGGGTGACAAGGTTGAACCACG |
| | | TCGTAAATTCATTGAAGAGAACGCTGTGTTTGTTAAAAACTTGGATAT |
| | | CTAA |
| 77 | DP9 Leucine | ATGGCTTATAATCATAAAGATATCGAACAGAAGTGGCAGCAATTCTG |
| | tRNA ligase | GAGCGACAATGAGACTTTTAAGACGGTCGAAGATGCAGACAAACCCA |
| | | AATATTATGCATTAGACATGTTCCCTTATCCATCAGGTCAAGGACTCC |
| | | ATGTGGGCCATCCTGAAGGATATACAGCAACAGATATTATGTCACGA |
| | | ATGAAACGGATGCAAGGTTACAAAGTACTTCATCCAATGGGATGGGA |
| | | TGCTTTTGGTCTTCCAGCAGAACAATATGCGATGAAGACGGGTAACA |
| | | ATCCGCGTGATTTTACAGCTAAGAATATTCAAAACTTTAAGCGTCAAA |
| | | TCCAATCACTTGGTTTTTCTTATGACTGGTCGCGAGAAGTTAATACAA |
| | | CTGATCCAGCTTACTACAAGTGGACTCAATGGATTTTTGAGCAACTCT |
| | | ACAAGAAGGGCTTAGCTTATGAAAAAGAAACGCTGGTAAACTGGGCT |
| | | CCTGATTTAATGGGTGGAACGGTAGTTGCTAACGAAGAAGTTGTGGA |
| | | TGGTAAGACAGAACGTGGTGGGTTCCCCGTTTATCGTAAACCAATGA |
| | | AACAATGGATTCTTAAAATTACAGCTTACGCCGACCGTTTGATTGACG |
| | | ATTTGGACCTGGTAGATTGGCCCGATAGTATTAAAGAAATGCAAAAA |
| | | AACTGGATTGGTCGTTCAGTGGGGGCTAGCGTCTTCTTTAATGTTGAA |
| | | GATAGCGAAAAACAAATTGAAGTATTTACAACGCGTCCAGATACATT |
| | | ATTTGGCGCAACATACTTGGTAATTTCACCAGAACATGACCTCGTTGA |
| | | CCAAATTACAACTCCAGAAAGTAAAGCTGCCGTTGAAGAATACAAGA |
| | | AAGCTGTTGCAACTAAATCAGATCTTGAACGGACGGATTTGAGTAAA |
| | | GATAAGACGGGAGTCTTTACGGGAGCATACGCGGTTAACCCTGTTAA |
| | | TGGTAAGAAAATTCCAGTTTGGATTAGTGATTACGTATTGGCTTCATA |
| | | CGGAACTGGAGCAGTGATGGCTGTTCCTGCTCATGATGGCCGTGACT |
| | | ACGAATTTGCTAAGAAATTCAAGATAGATATGGTGCCAGTTTATGAA |
| | | GGTGGCAATCTTGAAGATGGAGTATTGGACAGCGAAGGCGGGCTAAT |
| | | TAACTCTGGATTCCTAGATGGGATGGATAAGCAGACGGCTATTGATA |
| | | CCATGATTAGCTGGTTGGAAGAACATGGAGTTGGTCATAAGAAGGTT |
| | | AACTATCGTCTTCGTGACTGGGTCTTCTCTCGCCAACGCTACTGGGGT |
| | | GAACCAATCCCTGTAATTCATTGGGAAGATGGAGAAACAACTTTGAT |
| | | TCCTGAAGATGAATTGCCATTGAGACTCCCGGCTGCAACTGACATTCG |
| | | TCCTTCCGGTACCGGAGAAAGCCCATTAGCTAACCTAGATGATTGGGT |
| | | AAACGTAGTTGATGAAAATGGTCGTAAGGGTCGCCGGGAAACTAATA |
| | | CAATGCCACAATGGGCGGGTAGTTCATGGTACTTCCTCCGTTACGTTG |
| | | ATCCTAAGAATGATCAAAAGATTGCTGACGAAGATTTACTTAAAGAA |
| | | TGGTTACCAGTCGACTTATATGTTGGTGGAGCTGAACATGCGGTACTT |
| | | CATTTACTTTATGCACGTTTCTGGCACAAAGTTTTATATGATCTAGGA |
| | | GTTGTACCAACTAAGGAACCATTCCAAAAATTGGTCAACCAAGGGAT |
| | | GATTCTCGGTAGCAATCATGAGAAGATGTCTAAGTCAAAAGGGAACG |
| | | TGGTTAATCCAGATGATATTGTTGAGCGCTTTGGAGCGGATACTTTAC |
| | | GATTATACGAAATGTTCATGGGACCTCTGACAGAATCAGTCGCCTGG |
| | | AGTGAAGATGGGCTTAACGGAAGTCGTAAGTGGATTGACCGCGTCTG |
| | | GCGCTTGATGATTGACGACGAAAACCAATTGCGTGATCATATTGTTAC |
| | | TGAAAATGATGGCAGTTTGGATATGATTTATAACCAAACTGTTAAGA |
| | | AGGTAACTGATGATTATGAAAACATGCGCTTTAACACGGCTATTTCAC |
| | | AAATGATGGTCTTTGTTAATGAAGCATACAAGGCTGATAAACTTCCA |
| | | GCAGTATATATGGAAGGATTAGTTAAGATGTTAGCTCCAATTATTCCG |
| | | CACGTTGCTGAAGAACTTTGGAGTTTGCTAGGTCACGAAGGTGGTATT |
| | | TCATACGCTGAATGGCCAACATATGATGAAAGTAAGTTAGTAGAAGC |
| | | TACAGTTCAAGTCATTCTACAAGTTAATGGTAAAGTTCGGAGTAAAAT |
| | | TACCGTTGACAAGGATATCGCCAAAGAAGAACTTGAAAAATTAGCGT |
| | | TAGCTGATGCTAAGATTCAACAATGGACGGCAGATAAGACTGTTCGT |
| | | AAGGTAATTGTTATTCCTAACAAGATTGTTAATATCGTAGTAGGCTAA |
| 78 | DP9 Glucose-6- | ATGGCACATATTTCATTTGACAGTTCTAATGTTGCAGATTTTGTACAT |
| | phosphate | GAAAACGAACTTGCAGAAATCCAACCACTTGTTACAGCTGCTGATCA |
| | isomerase | GATTTTACGTGATGGCTCTGGCGCTGGTAGTGATTTCCGTGGATGGAT |
| | | CGATTTACCATCAAATTATGATAAGGACGAATTTGCCCGTATCAAGA |
| | | AAGCCGCTGATAAGATCCGCAATGACTCAGAAGTATTCGTTGCTATC |
| | | GGTATTGGTGGTTCATATTTGGGTGCTCGTGCAGCCATTGATTTCTTG |
| | | AACAACACTTTCTACAATCTTCTTACTAAAGAACAACGTAATGGTGCT |
| | | CCTCAAGTAATCTTCGCTGGTAACTCAATTAGTTCAACTTACCTTGCT |
| | | GACGTATTGAACTTAATCGGGGACCGTGACTTCTCAATTAACGTAATT |
| | | TCTAAGTCAGGTACAACTACAGAACCAGCTATTGCATTCCGTGTTCTT |
| | | AAAGAAAAACTAATCAAGAAGTACGGTGAAGAAGAAGCTAAGAAAC |
| | | GTATCTATGCAACAACTGACCGTGCTAAAGGCGCCCTAAAGACAGAA |
| | | GCTGATGCAGAAAACTATGAAGAATTCGTAGTTCCTGATGACATTGG |
| | | TGGTCGTTTCTCTGTTCTTTCAGCTGTTGGTTTATTACCAATCGCGGTT |
| | | GCCGGTGGCGATATTGACCAATTGATGAAGGGTGCTGAAGATGCAAG |
| | | CAACGAATACAAGGATGCTGATGTTACAAAGAACGAAGCATACAAGT |
| | | ACGCTGCTTTACGTAACATCCTTTATCGTAAGGGCTACACAACAGAAC |
| | | TTCTTGAAAACTACGAACCAACACTTCAATACTTCGGCGAATGGTGG |
| | | AAGCAATTGATGGGTGAATCAGAAGGTAAAGATCAAAAGGGTATCTA |
| | | CCCATCTTCTGCTAACTTCTCAACTGACTTACATTCACTAGGACAATA |
| | | CATCCAAGAAGGTCGTCGCAATTTAATGGAAACAGTTATCAATGTTG |
| | | AAAAGCCTAACCATGACATCGACATTCCTAAGGCTGACCAAGACCTT |
| | | GATGGATTACGTTATCTCGAAGGTCGCACAATGGACGAAGTTAACAA |
| | | GAAAGCTTACCAAGGTGTAACTCTTGCTCATAACGACGGTGGTGTTCC |
| | | AGTTATGACGGTTAACATTCCTGATCAAACAGCTTACACATTAGGCTA |
| | | TATGATTTACTTCTTCGAAGCAGCTGTTGCTGTATCTGGTTACTTGAAC |
| | | GGAATTAATCCATTCAACCAACCAGGTGTTGAAGCATACAAGTCAAA |
| | | TATGTTTGCATTACTTGGTAAACCAGGTTATGAAGATAAGACAGCTGA |
| | | ATTAAACGCTCGTCTATAA |
| 79 | DP9 | ATGAGTTGGGAAGATTCTGTCAAAGAATGGCAAGATTATGCAGATTT |
| | Phospho- | AGATTTTAATTTAAAAAAAGAATTAGCAACTTTAGCTGAAGATAAAG |
| | glucomutase | ATGCTTTAAAAGAAGCCTTTTATGCTCCAATGGAATTTGGTACAGCAG |
| | | GAATGCGTGGCGTAATGGGCCCTGGTATCAACCGGATGAATATCTAT |
| | | ACGGTTCGTCAAGCAACAGAAGGTTTAGCTAATTTTATGGATACCTTA |
| | | GATTTTACTGATAAGAAACGGGGAGTGGCGATCAGTTTTGATTCCCGC |
| | | TATCACTCACAAGAGTTTGCTTTAGCAGCAGCTGGTGTTTTAGGTAAG |
| | | CATGGTATTCCAAGTTTTGTTTTTGATAGTATGCGTCCCACTCCAGAA |
| | | TTATCATATACAGTACGTGAGTTAAACACTTATGCTGGAATCATGATT |
| | | ACTGCTAGTCATAATCCTAAACAATATAATGGATATAAGATTTATGGT |
| | | CCTGATGGCGGACAAATGCCACCAATGGAATCTGATAAGATTACAGA |
| | | ATATATTCGCCAAGTAACTGACATCTTTGGTGTTGAAGCTCTTACTCA |
| | | AAGTGAATTAAGAGCTAAGGGCTTAATGACCATTATTGGTGAAGACA |
| | | TTGACCTCAAGTATCTTGAGGAAGTTAAGACGGTATCAATTAATCATG |
| | | AACTAATCCAGCGCTTTGGTGCAGACATGAAGTTGATCTACTCACCAT |
| | | TACATGGTACTGGAAAAGTAGTTGGTGGACGTGCGTTAGAAAATGCT |
| | | GGTTTTAAGGATTACACTATGGTCCCTGAACAAGCAATTGCTGACCCA |
| | | GAATTTATTACAACGCCATTCCCTAACCCAGAATTCCCACAAACTTTT |
| | | GATTTGGCTATTGAATTAGGTAAAAAGCAAGATGCTGACCTTTTGATT |
| | | GCCACTGATCCGGATGCCGATCGTTTGGGAGCTGCCGTTCGTTTACCA |
| | | AATGGTGACTACAAATTATTGACAGGGAACCAAATTGCAGCCTTGAT |
| | | GTTAGAATACATCTTAACTGCGCATGATGCAGCAGGTGACTTGCCAG |
| | | GTAACGCAGCTGCCGTTAAGTCAATTGTTTCTAGTGAACTAGCAACCA |
| | | GAATTGCCGAAGCCCATCATGTAGAAATGATTAACGTTCTAACTGGG |
| | | TTTAAGTACATTGCTGACCAAATTAAACATTACGAAGAAAATGGCGA |
| | | CCATACCTTTATGTTTGGTTTCGAAGAAAGTTATGGCTATCTTGTTCG |
| | | GCCATTTGTTCGCGATAAAGATGCCATCCAAGGAATTGTCCTATTGGC |
| | | TGAAATTGCTGCTTATTATCGTAGTAAGGGGCAAACCTTATATGACGG |
| | | TCTTCAAAACTTATTTACTACTTACGGATATCATGAAGAAAAGACCAT |
| | | TTCAAAAGATTTCCCTGGAGTTGACGGTAAAGAAAAAATGGCTGCCA |
| | | TTATGGAAAAGGTTCGTGAAGAACGCCCAAGTCAATTTGATCAGTAC |
| | | AAGGTATTAGAAACTGAAGACTTCTTAGCTCAAACTAAGTATGAAGC |
| | | AGATGGATCTACCCAAGCTATCAAATTACCAAAAGCGGATGTTTTGA |
| | | AATTTACATTAGATGATGGTACTTGGATTGCAATTCGTCCTTCTGGAA |
| | | CAGAACCAAAAATTAAATTCTATATTGGTACAGTTGGCGAAGATGAA |
| | | AAAGATGCTTTGAATAAGATTGATGTTTTTGAAACAGCTATTAATGAA |
| | | CTTATAAAATAA |
| 80 | DP9 2- | ATGCACCGTATTTTAATTGCCAACCGAGGCGAAATTGCGACCCGAATT |
| | oxoglutarate | ATTCGGGCAACGCATGAACTCGGAAAAACAGCTGTAGCAATTTATGC |
| | carboxylase | TAAAGCGGATGAATTTTCTATGCATCGTTTTAAAGCAGATGAAGCTTA |
| | small subunit | CCAAGTTGGTGAAGATAGTGATCCAATTGGAGCATATTTAAATATTG |
| | | ATGACATTATTCGTATTGCAAAAGAAAATAATATTGATGCAATTCACC |
| | | CCGGCTATGGATTTTTGTCGGAAAATGCTGTATTTGCGCGAGCAGTTG |
| | | AAGCAGCTGGGATTAAGTTCATTGGACCTCGACCCGAATTACTAGAA |
| | | ATGTTTGGTGATAAATTACAAGCTAAAAATGCAGCCATTAAGGCCGG |
| | | TGTACCAACTATTCCGGGAACGGAAAAACCAGTTAAAGATGTCGATG |
| | | ACGCGCTAAATTTTGCAGAGCAATTTGGCTATCCTATATTTGTTAAGT |
| | | CAGCGGCAGGTGGCGGCGGAAAAGGGATGCGGATTGTACATCATCAA |
| | | CAAGAGATGCGCGAAGCATTTAAGATGGCTCAGTCAGAAGCTTCTTC |
| | | GTCTTTTGGTGACGATGAAATTTACTTAGAACGTTACTTAGTTGATCC |
| | | AATCCATATTGAGGTTCAAGTAGTTGCGGATGAACACGGTGAGATGG |
| | | TTCATTTGTATGAACGAAATTCATCGATTCAGCGACGCCATCAAAAAA |
| | | TCATTGAATTTGCTCCAGCAGTGGGAATTTCTGCCACCGTCCGTGATC |
| | | AAATAAGAAAAGCTGCTTTAAAATTATTGAAGTCGGTCAATTATAGT |
| | | AACGCTGCAACCATTGAGTTTTTGGTAGAAGGTAATCAATTTTACTTT |
| | | ATGGAAGTGAATCCACGAATTCAGGTTGAACATACAGTTACCGAAGA |
| | | AGTCACGGGAATCGATATTGTGCAAACCCAAATTAAGGTTGCTGAAG |
| | | GTCAAAGATTACACGAAGAAATCGGTGTTCCTCAACAAGCCCAAATT |
| | | GAAGCTGTGGGAGTGGCAATTCAAGCCCGAATTACCACTGAAGATCC |
| | | AATGAATAACTTTATTCCAGATGTCGGTAGAATCCAGACGTATCGTTC |
| | | ACCTGGTGGAACAGGTGTGAGATTGGATGCTGGAAATGCCTTTGCTG |
| | | GAGCCATTGTAACTCCGCATTATGATTCACTTCTGACCAAGGCAATTG |
| | | TCCATGCGCCAACCTTTGACGAAGCCTTGGTAAAGATGGATCGAGTG |
| | | CTCAATGAATTTGTAATTGCTGGGGTTAAAACTAATATTCCATTTTTA |
| | | AAGAAATTAATTCATCATCCTATTTTTAGATCGGAATTAGCTCCGACA |
| | | ACCTTTGTGGATGAGACACCAGAACTCTTTGATTTAAAAGCTGAAACT |
| | | CCGGTAGTTACTCAACTTTTGAGTTACATTGCTAATACTACTATCAAT |
| | | GGTTATCCAGGCTTAGAAAAGCAGAATCCAGTAGTGTTAACTCGGCC |
| | | AGTCCGTCCACATTTTGAAGCACAAGTACCGCATGAAAATGCGAAAC |
| | | AGATCTTGGATAGTAAGGGACCTGATGCCATGATCAATTGGCTGTTA |
| | | AAACAAAAGCAGGTCTTGCTAACCGATACGACCATGCGGGATGCCCA |
| | | TCAATCATTATTTGCTACGCGAATGCGGACCAAAGACATGGTAGAAA |
| | | TTGCCGATCAAGTCCAGAAAGGTCTGCCTAACCTATTTTCAGCTGAAG |
| | | TTTGGGGCGGTGCGACCTTTGATGTTGCTTATCGGTTCCTAGGTGAGG |
| | | ATCCATGGGAAAGACTCCAACAATTGCGGGCTAAAATGCCAAATACG |
| | | ATGCTCCAAATGCTTTTACGTGGGTCAAATGCAGTAGGGTATCAAAAT |
| | | TATCCAGACAACGCCATTGACGAATTTATTCGATTGGCTGCCAAAAAT |
| | | GGAATTGATGTTTTCCGAATCTTTGATTCTCTTAATTGGGTGCCACAG |
| | | CTTGAAGAATCTATCCAACGGGTGCGTGATAATGGAAAAGTGGCTGA |
| | | AGCAGCCATGGCATATACTGGCGATATTTTAGATACTAATCGTACTAA |
| | | ATATAATTTGAAATATTATGTGGATTTGGCTCAAGAACTCCAAGCAGC |
| | | AGGTGCTCATATTATTGGAATCAAAGATATGTCAGGAATTTTAAAACC |
| | | ACAAGCTGCTTATGCATTAATTTCAGAGTTAAAAAATCATCTGGATGT |
| | | GCCAATTCATTTGCATACGCACGATACTACAGGCAACGGCATTTTCTT |
| | | ATATTCTGAAGCAATACGAGCTGGAGTTGATGTGGTCGACGTTGCCA |
| | | CTTCTGCGCTAGCGGGAACGACTTCTCAGCCTTCAATGCAGTCTCTTT |
| | | ACTATGCGTTGTCTAATAACCAGCGCCAACCAGATTTAGATATTCAAA |
| | | AAGCAGAAAAACTAGATGAATATTGGGGCGGAATTCGACCATATTAC |
| | | GAAGGATTTGGCACCCAATTAAATGGACCACAAACTGAAATTTATCG |
| | | AATTGAAATGCCTGGTGGACAGTATACCAACCTTCGCCAGCAAGCTA |
| | | ACGCAGTCCATTTGGGTAAGCGTTGGGATGAGATTAAGGAAATGTAC |
| | | GCAACCGTCAATCAAATGTTTGGCGATATTCCAAAGGTTACGCCTTCT |
| | | TCTAAAGTAGTTGGCGATATGGCACTATTCATGGTCCAAAATGATTTG |
| | | ACGCCTGAAATGGTAATGAACGATAAGGGACAATTAAGTTTTCCCGA |
| | | ATCAGTGGTAAACTTTTTCCGTGGTGATTTAGGACAACCGGCGGGTGG |
| | | TTTTCCAAAACAGCTCCAAAAGGTGATTCTAAAAGAGCAAGCCCCAT |
| | | TGACAGTACGACCAGGAGCTTTAGCCGATCCAGTTGATTTTGATCAAG |
| | | TTCGTAAACAGGCAACTAAGGTTTTAGGTCACCAAGCAAGTGATGAA |
| | | GAAGTTATGTCGTTTATTATGTATCCAGATGTGATGACCGAATACATT |
| | | CAACGTCAAAATGAATATGGTCCAGTACCATTATTAGATACTCCAATC |
| | | TTTTTCCAAGGCATGCATATTGGCCAACGCATTGATTTACAATTGGGA |
| | | CGCGGAAAATCGGTCATTATTGTCCTTCGAGAAATTAGTGAAGCAGA |
| | | TGAGGCGGGCCAAAGGTCACTTTTCTTTGATATAAATGGACAAAGTG |
| | | AAGAAGTGATTGTTTATGATGTTAATGCGCAGGTAACGAAAGTAAAG |
| | | AAGATTAAAGCTGATCCGACTAAAGCCGAACAGATTGGCGCTACTAT |
| | | GGCGGGCTCGGTCATTGAAGTCCAAGTAGAAGCGGGCCAAAAGGTCC |
| | | AGCGAGGTGATAACTTAATTGTCACTGAGGCGATGAAAATGGAGACC |
| | | GCGTTAAGAGCACCTTTCGACGCAACCATTAAGAAGATTTATGCTACC |
| | | CCTGAAATGCAAATCGAGACGGGGGATTTATTGATTGAACTAGAAAA |
| | | GGAGTAA |
| 81 | DP3 Glycine | ATGTCAACATTTTTATTAGAAATTGGACTTGAAGAAATACCAGCTCAT |
| | tRNA ligase | TTGGTAACCAGTTCAGAGAATCAGTTAATTGAAAGAACTAAAAAGTT |
| | beta subunit | CTTATCAGAGCATCGTTTAACAGTAGGTGATATTAAACCATATTCAAC |
| | | ACCGCGACGTCTGGCTGTCGTTTTGACAGATGTTGCTGAAACATCAGA |
| | | AAGTTTAAGCGAAGAAAAGCGTGGACCATCTGTTGACCGTGCACAAG |
| | | ACGAAAACGGTAATTGGACAAAGGCAGCATTAGGTTTTGCACGTGGT |
| | | CAAGGTGCTAATCCTGAAGCATTTGAAATTAAAGATGGATATGTTTG |
| | | GCTAACAAAACGTACTGCTGGTGTAGCCGCGAATGAAATTTTAGCTA |
| | | AAATTGGTGATGAAGTTGTCGCCCAAATGAAATTTTCAACTTATATGA |
| | | AGTGGGCTAATCACAGCTTTTTGTATGTTCGACCTATTCGTTGGCTCG |
| | | TAGCACTTCTTGATAGTGAAGTCATTTCTTTCAACGTGTTAGATATTA |
| | | CCACAGATCGTTTCACACGTGGTCATCGTTTTTTGTCTTCAGAACATG |
| | | TTGAAATATCTTCTGCAGATAATTATGTAACGACTTTGCAGGGTGCTA |
| | | ACGTGGTTGTTGATGCTACAGTGCGCAAAAATGAAATTCGATCGCAG |
| | | TTGAATGCAATTGCTGAAGCTAATGGTTGGGTTCTGCAACTTGAGACC |
| | | GATGCGGCGCAAGATTTGTTGGAAGAAGTTAATAACATTGTTGAGTG |
| | | GCCAACAGCGTTTGCTGGCAGTTTCGATGAGAAATATTTAGAAATAC |
| | | CAGATGAAGTTTTGATTACATCAATGCGCGAACATCAGCGTTTCTTCT |
| | | TTGTGACGAATGAAAAAGGACAATTATTGCCACACTTTTTGTCAATAA |
| | | GAAATGGTAACCGTGAGCATCTAAACAACGTTATTGCTGGAAATGAA |
| | | AAAGTATTGGTAGCAAGGTTAGAAGATGCCGAATTCTTCTATCATGA |
| | | AGACCAAACCAAATCAATTTCTGATTACATGACTAAAGTTAAAAAGT |
| | | TAGTCTTCCATGAAAAAATTGGTACGGTGTATGAACACATGCAACGC |
| | | ACTGGTGCTTTGGCTTCAGCAATGGCGGTGGTTTTGAAGTTTGATGAA |
| | | GTACAACAGGCTGATTTGACCCGTGCATCAGAAATTTATAAATTTGAT |
| | | TTGATGACCGGTATGGTTGGTGAATTTGATGAACTTCAAGGCATTATG |
| | | GGTGAGCATTATGCCAAGCTTTTTGGCGAAGATGATGCGGTTGCAAC |
| | | AGCCATTCGAGAGCATTATATGCCAACTTCAGCTAATGGTGAGGTTGC |
| | | GCAATCTGAAATTGGTGCTTTGTTGGCCGTTGCGGATAAACTTGATAG |
| | | CATTGTGACGTTTTTTGCTGCTGGATTAATACCAAGTGGTTCTAATGA |
| | | TCCTTATGGCTTACGACGTGCAGCTACTGGCATCGTGCGTACATTGGT |
| | | GGATAAAAAATGGCATATTGATTTGCGGCCTTTGCTAGCTGATTTTGT |
| | | GCAACAGCAAGGTAAGGTAACTGACACCGATTTAACGACATTTGTTG |
| | | ATTTCATGTTGGATCGTGTTCGTAAATTATCGTTGGATGCTGGAATAC |
| | | GTCAAGATATTGTCATTGCTGGATTAGGCAACGTTGATAGAGCTGATA |
| | | TCGTATATATTAGTCAGCGAGTCGAAGTTTTGTCCCAACATAGTGGTG |
| | | ATGGCAATTTCCGAGATGTAATTGAGGCACTGACTCGTGTGGATCGCT |
| | | TAGCCGTAAAGCAAGTAACTAATGCAACGGTTGATCCTGCTAAGTTT |
| | | GAAAATCAATCTGAAAAGGACCTATATCAAGCAACGTTAACGCTTGA |
| | | TTTAAATACTTTGATGCATGACGGTGCAGAAAATCTCTACATGGCCTT |
| | | AGCAAATTTGCAAAAACCAATTGCGGCTTATTTTGATGAAACCATGGT |
| | | TAACGCTGAAGATGAATCTGTTAAAGATAATCGATATGCGCAGCTGA |
| | | ACGTCATACAACGACTAACCAACGGATTAGGAGATTTGACGCAAATC |
| | | GTCATTAAGTAA |
| 82 | DP3 Glutamine | ATGGCTCGTAAAACATTTACCAAAGAAGAAATTAAACAAATTGTTGT |
| | synthetase | TGATGAAAATGTAGAATTCATTCGTGTAACATTCACTGATGTCTTAGG |
| | | TGCGATTAAAAACGTTGAAGTACCAACTTCTCAATTAGATAAGGTGCT |
| | | TGACAACAATTTAATGTTTGACGGTTCATCAATCGAGGGATTTGTTCG |
| | | TATCAATGAATCAGATATGTATCTTTACCCCGATTTATCAACATTTAT |
| | | GATTTTCCCATGGGCAACGGATGGTCATGGTGGTAAAGTGGCCCGCTT |
| | | GATTGCCGACATTTATACTGCTGATCGTGAGCCATTTGCTGGAGACCC |
| | | CCGTCATGCGTTACGTTCGGTACTCGCTGACGCGCGTGAAGCTGGGTT |
| | | TACGGCGTTTAATGTCGGGACAGAACCTGAATTTTTCTTGTTTAAACT |
| | | TGATGAAAAAGGCAACCCAACCACAGAGTTAAACGACAAAGGTGGTT |
| | | ATTTTGACCTAGCACCATTGGATATGGGTGAAAATGTTCGTCGTGAAA |
| | | TTGTTTTGACTTTGGAAAAAATGGGCTTTGAAATTGAAGCTGCTCACC |
| | | ACGAAGTTGCCGAAGGACAGCATGAAGTAGACTTTAAATACGCTTCA |
| | | GCTCTTGAAGCCGCTGACAACATTCAGACGTTTAAGTTGGTTGTTAAA |
| | | ACCATCGCACGCAAGAATGGTTACTATGCTACCTTTATGCCAAAGCCT |
| | | GTTGCAGGTATTAACGGATCCGGTATGCACACAAACATGTCATTATTT |
| | | ACAAAAGATGGTAACGCATTTGTTGATACATCGGATGAAATGGGCTT |
| | | GTCAAAAACAGCATATAACTTCTTGGGTGGTATTTTAGAACATGCGAC |
| | | TGCGTTTACAGCGCTTGCAAACCCAACAGTTAACTCATACAAGCGCTT |
| | | GACACCAGGATTCGAAGCACCTGTTTATGTTGCATGGTCAGCATCAA |
| | | ATCGTTCACCAATGGTTCGAGTTCCGGCCTCACGTGGTAATTCAACAC |
| | | GTTTGGAACTTCGTTCAGTTGACCCAACAGCTAATCCTTATACTGCAT |
| | | TGGCAGCCATTTTGGCTTCAGGACTGGATGGGATCAAGCGTGAATTA |
| | | GAGCCTTTGGCCTCAGTTGATAAAAATATTTATTTGATGGATGAGGTC |
| | | GAACGGGAAAAGGCAGGCATTACAGACTTACCAGATACTCTGTTGGC |
| | | TGCAGTTCGTGAGTTGGCGGCTGATGATGTTGTTCGTTCAGCTATTGG |
| | | AGAACATATTGCTGATAAGTTTATTGAAGCAAAGAAGATTGAATACA |
| | | CATCATATCGTCAGTTTGTTTCTGAATGGGAAACAGATTCTTATCTTG |
| | | AAAATTACTAA |
| 83 | DP3 DNA gyrase | GTGTTCGCAGATTATATCTGTTCACACGCTAATAATATGGCAGAGAAT |
| | subunit B | ATCGAAAATGAAGCATTGGAGAACATTGATGGCATCGTAACCGATGA |
| | | TACCGAAATCCGTCAAGCAAGCACCGTTCATGCAGCAGCAGGCGCTT |
| | | ACAATGCTGATCAGATTCAAGTTTTGGAAGGATTGGAAGCTGTCCGC |
| | | AAACGCCCTGGCATGTACATTGGTACGACCACAGCGCAAGGCTTGCA |
| | | CCATTTGGTATGGGAAATTGTTGATAACGGGATTGATGAGGCATTAG |
| | | CAGGGTTTGCGTCACATATTACGGTCACAATCGAAAAGGATAACTCA |
| | | ATCACGGTAACCGATGACGGCCGTGGTATTCCTGTCGACATTCAAACT |
| | | AAAACGGGTAAGCCAGCTCTTGAAACTGTCTTTACGGTATTACACGCC |
| | | GGTGGTAAATTTGGCGGTGGCGGTTATAAAGTATCTGGTGGATTACA |
| | | CGGTGTTGGAGCTTCTGTTGTCAATGCCTTGTCAACGGATTTGGACGT |
| | | TAGAGTTGTTCGTGATAATACTGTTTATTACATGGACTTCAAAGTGGG |
| | | ACGCGTCAACACACCGATGAAACAATTGACGGAAAAGCCCACTATTG |
| | | AGCGTGGTACAATTGTTCATTTTAAGCCCGATGCAGATATTTTCCGTG |
| | | AAACAACAGTTTATAACTACAACACATTACTAACACGTGTGCGCGAA |
| | | TTGGCCTTTTTGAATAAAGGTTTGCGCATTTCGATTACAGATAATCGA |
| | | CCTGAAGAAGCTGTTTCTGAAAGCTTTCATTTTGAAGGTGGGATTAAA |
| | | GAATACGTCAGCTATTTGAATAAGGACAAGACTGCTATTTTCCCTGAA |
| | | CCTGTTTACGTTGAGGGTGAAGAAAATGGCATTGTAGTGGAAGCTGC |
| | | CTTACAGTACACTACCGATATTAAAGACAATCTGCGGACGTTTACTAA |
| | | CAATATCAATACCTATGAAGGTGGGACGCACGAAACTGGCTTTAAAA |
| | | CAGCCTTAACACGTGTAATCAATGATTACGCTCGTAAAAATGGTCAG |
| | | CTCAAAGATAATGCAGAAAGTTTGACAGGGGAAGATGTGCGCGAAG |
| | | GCATGACTGCTATCGTGTCAATCAAGCACCCAGATCCACAATTTGAA |
| | | GGACAAACCAAAACTAAATTAGGTAACTCCGATGCACGTCAAGCAAC |
| | | GGATCGGATGTTCTCAGAAACGTTCAGTCGTTTCATGATGGAAAATCC |
| | | AGCAGTTGCCAAGCAAATTGTTGAAAAAGGTGTCTTAGCCCAAAAAG |
| | | CACGATTGGCTGCCAAGCGTGCACGCGAAATGACACGCAAACAATCT |
| | | GGTTTGGAAATTGGTAATTTGCCAGGTAAATTAGCTGATAATACCTCA |
| | | AATGATCCTGAAATTTCAGAATTATTTATTGTTGAGGGTGATTCAGCC |
| | | GGTGGTTCAGCTAAGCAAGGACGTAACCGTTTGACGCAAGCTATTTT |
| | | GCCAATTCGAGGCAAAATTTTAAATGTTGGGAAAGCCTCATTGGATC |
| | | GGGTGTTAGCCAACGAAGAAATTCGATCATTGTTTACAGCAATGGGA |
| | | ACTGGATTTGGTGAGGACTTTAATGTTGAAAAAGCCAATTATCACAA |
| | | AGTCATTATTATGACAGATGCCGATGTCGATGGCGCCCATATTCGAAC |
| | | ACTATTGTTAACGCTATTTTATCGTTATATGCGACCACTTGTTGACGC |
| | | AGGCTATATTTATATTGCGCAGCCACCGCTTTACGGTGTTGCCTTAGG |
| | | CAATAATAAATCAATGACGTACATTGATTCTGATGAAGAACTTGAAG |
| | | ACTATTTGTCACAATTGCCATCTAATATTAAACCAAAAGTTCAACGTT |
| | | ATAAGGGACTAGGGGAAATGGATTACGATCAACTAGCAGATACAACC |
| | | ATGGATCCGCAGAATCGTCGTTTGCTACGTGTTGACCCAACTGATGCT |
| | | GAAGAAGCCGAAGCAGTTATTGATATGTTAATGGGTGGGGATGTACC |
| | | ACCACGTCGTAAGTTTATTGAAGACAATGCTGTCTTTGTTGAGAACTT |
| | | GGATATTTAA |
| 84 | DP3 Leucine | ATGATTTTCGTCAACGAAGCTTACAAAACCGATGCTGTGCCGAAAGC |
| | tRNA ligase | GGCGGCGGAAAACTTCGTACAGATGCTGTCCCCACTGGCACCGCATT |
| | | TGGCAGAAGAACTGTGGGAACGACTTGGTCATACCGATACGATTACG |
| | | TATGAACCATGGCCAACGTACGATGAGGCTTGGACCATAGAATCCGA |
| | | AGTGGAAATCGTCGTGCAAGTGAACGGCAAAATCGTAGAACGCACGA |
| | | AAATTTCCAAAGACCTGGATCAAGCAGCGATGCAAGAACACAGCTTA |
| | | AGCCTGCCGAATGTTCAGCAGGCTGTGGCTGGGAAGACGATCCGCAA |
| | | AGTGATTGCGGTGCCAGGCAAGCTGGTGAATATCGTCGTTGGATAA |
| 85 | DP3 Glucose-6- | ATGGCACACATTACATTTGACACAAAGAACATTGAGAATTTTGTTGCA |
| | phosphate | CCATACGAATTGGACGAAATGCAACCATTAATTACGATGGCTGACCA |
| | isomerase | ACAATTGCGCAATCGTACGGGCGCTGGTGCAGAATATTCTGATTGGTT |
| | | GACTCTACCTACTGATTACGACAAGGAAGAATTTGCACGTATTCAAA |
| | | AGGCGGCGCAACAAATTCAATCTGATTCAAAGATTTTGGTTGTCATTG |
| | | GTATTGGTGGTTCATATTTGGGCGCGAAGATGGCGGTTGATTTCTTGA |
| | | ATCCAATGTTTAATAATGAATTGTCGGATGACCAACGTCAAGGTGTTA |
| | | AAATTTATTTTGCTGGTAACTCAACTTCTGCAGCTTACTTAAATGATTT |
| | | AGTTCGTGTCATTGGTGATCAAGACTTTTCTGTCAACGTTATCTCAAA |
| | | GTCTGGCACAACAACGGAACCATCAATCGCTTTCCGTGTGTTTAAACA |
| | | ATTGTTAGAGAAAAAGTATGGTTCTGATGCTGCTAAGAAGCGTATCT |
| | | ATGCCACAACAGATGCCAATCGTGGTGCTTTGCACGATGAAGCAGCG |
| | | GCTTCAGGTTATGAAACATTCACAATTCCTGATGGTGTCGGTGGTCGC |
| | | TTCTCTGTTTTGACAGCTGTTGGCTTGTTGCCAATTGCTGCTTCAGGCG |
| | | CTGATATCCAAAAATTGATGGACGGCGCTCGTGATGCGCAAAACGAA |
| | | TATACTGATTCTGATTTGAAAAAGAACGAGGCATATAAATATGCAGC |
| | | CGTTCGTCGTATTTTGTATGATAAGGGTTATACAACAGAATTGTTGAT |
| | | TAACTGGGAACCTTCAATGCAATATTTGTCAGAGTGGTGGAAGCAAT |
| | | TGATGGGCGAGTCTGAAGGTAAAAATCAAAAGGGTATCTATCCATCT |
| | | TCAGCTAACTTCTCAACCGACTTGCACTCACTTGGACAATATATTCAA |
| | | GAAGGACGCCGTGATTTGTTTGAGACGGTGGTTAAGTTAGACAATCC |
| | | TGTATCTAATTTGGACCTACCACATGAAGAAGGCAACAATGATGGTTT |
| | | GCAATATTTGGAAGGTATCACGATCGATGAAGTGAACACCAAAGCAT |
| | | CTCAAGGGGTTACTTTGGCTCACGTTGATGGTGGTGTGCCTAACTTGG |
| | | CTGTTCACTTGCCAGCACAAGATGCTTATTCACTCGGTTACATGATTT |
| | | ACTTCTTTGAAATGGCTGTTGGGGCGTCTGGTTATACGTTTGGTATTA |
| | | ACCCATTCAACCAACCGGGTGTCGAAGCCTATAAGACAGCTATGTTT |
| | | GCACTATTAGGTAAGCCTGGCTATGAGGAAGCGACAAAAGCATTCCG |
| | | TGCCCGCTTAGACAAATAA |
| 86 | DP3 Beta- | ATGACTAAATTTTCAGATATTAAAGGTTTTGCCTTTGATTTAGATGGG |
| | phospho- | GTTATTGCTGATACGGCGCGTTTCCATGGTGAAGCTTGGCATCAAACA |
| | glucomutase | GCTGATGAGGTTGGCACAACTTGGACACCAGAATTGGCTGAAGGTTT |
| | | GAAGGGCATTAGTCGTATGGCTTCCTTGCAAATGATTTTGGATGCTGG |
| | | GGATCATGCCGATGATTTTTCGCAAGCAGATAAAGAAGCATTAGCAG |
| | | AAAAGAAAAATCATAATTATCAACAACTTATTTCAACATTGACGGAA |
| | | GATGATATTTTGCCTGGCATGAAAGATTTTATTCAATCAGCCAAGGCA |
| | | GCCGGCTATACAATGTCGGTGGCATCAGCTTCTAAAAACGCACCAAT |
| | | GATTCTAGATCATTTGGGATTGACCAAGTATTTTGTCGGCATTGTTGA |
| | | TCCCGCCACTTTGACAAAGGGAAAACCTGATCCTGAAATCTTCGTTCG |
| | | TGCTGCGGAAGTCTTACATTTAAATCCAGAAAATGTTATTGGATTGGA |
| | | AGATTCAGCTGCTGGTATTGTGTCAATCAATGGCGCAGGTGAGACAT |
| | | CACTAGCCATTGGTAACGCAGATGTTTTGTCAGGAGCGGACTTGAATT |
| | | TTGCGTCTACTTCAGAAGTGACCTTAGCAAATATTGAAGCTAAAATGC |
| | | AATAG |
| 87 | DP3 2- | ATGTTTAAAAAAGTGCTTGTTGCTAATCGTGGTGAAATTGCGGTTCGC |
| | oxoglutarate | ATCATTCGAACGCTCAAAGAAATGGGGATTGCTTCAGTCGCTATTTAC |
| | carboxylase | TCGACAGCCGATAAAGATAGTTTACACGTACAAATCGCTGACGAAGC |
| | small subunit | GATTGCTGTGGGGGGACCGAAACCTAAAGATTCATACTTAAATATGA |
| | | AAAATATTTTAAGTGCAGCCCTGCTGTCGGGAGCAGAGGCAATTCAT |
| | | CCAGGATATGGCTTTTTAGCTGAAAATACATTGTTTGCTGAAATGGTT |
| | | GGCGAAGTTGGTATTAAATGGATTGGGCCTAGGCCAGAAACAATTGA |
| | | GTTAATGGGTAACAAAGCTAACGCACGTGAAGAAATGCGGCGTGCCG |
| | | GCGTACCAGTAATTCCAGGTTCAGAGGGATTTATCCGTGATTTTCATG |
| | | AAGCAAAAACGGTTGCTGATAAAATTGGCTATCCTTTGTTGCTAAAA |
| | | GCTGCCGCTGGTGGTGGTGGTAAAGGCATGCGTTTTGTTTACGGTGAG |
| | | GATGAGTTATCAGATAAATTTGATGATGCTCAAAACGAAGCGCGTGC |
| | | TTCGTTTGGCGATGATCACATGTATATTGAAAAAGTTATGTCACGTGT |
| | | TCGCCACATTGAAATGCAAGTGTTTCGTGATGAGAATGGTCATGTTGT |
| | | TTACTTGCCAGAACGAAATTGCTCATTGCAACGCAATAATCAAAAGG |
| | | TGATTGAAGAATCACCAGCTACGGGTGTAACGCCTGAAATGCGTGCG |
| | | CATCTTGGCGAAATTGTTACTAAAGCCGCAAAAGCATTGGCGTATGA |
| | | AAATACTGGAACCATTGAATTTTTGCAAGATCGCGATGGTCATTTCTA |
| | | CTTTATGGAAATGAACACACGTATTCAAGTAGAACATCCAGTTTCTGA |
| | | AATGGTAACGGGATTAGATTTAATTAAGTTACAAATTCAAGTTGCTGC |
| | | AGGCTTAGATTTACCGGTGGTTCAAGATGACGTGATCGTTCAAGGCC |
| | | ACTCTATCGAAGTACGTTTGACGGCTGAGCAGCCAGAAAAACACTTT |
| | | GCACCTAGTGCTGGAACGATTGATTTTGTTTTTTTGCCAACTGGTGGA |
| | | CCGGGTGTTCGTATTGATTCAGCCTTATTTAATGGCGATAAAATTCAA |
| | | CCATTTTACGATTCTATGATTGGCAAATTAATTGTTAAGGCCGATGAT |
| | | CGTGAAACAGCCATGAGAAAGATTCAACGTGTGGTTGATGAAACTGT |
| | | TGTACGTGGTGTAGCAACGAGCCGTAATTTTCAAAAAGCTCTGTTAGC |
| | | TGATCCACAGGTTCAACGTGGCGAATTTGACACACGTTATTTGGAAAC |
| | | TGAATTTTTACCGAGATGGACACAAACATTGCCAGATAATCAATAA |
| 88 | DP1 Glutamine- | ATGAGCAAGCCCACTGTCGACCCTACCTCGAATTCCAAGGCCGGACC |
| | tRNA ligase | TGCCGTCCCGGTCAATTTCCTGCGCCCGATCATCCAGGCGGACCTGGA |
| | | TTCGGGCAAGCATACGCAGATCGTCACCCGCTTCCCGCCAGAGCCCA |
| | | ACGGCTACCTGCACATCGGTCATGCCAAGTCGATTTGTGTGAACTTCG |
| | | GCCTGGCTCAGGAGTTCGGTGGCGTTACGCACCTGCGTTTCGACGACA |
| | | CCAACCCGGCCAAGGAAGACCAGGAATACATCGACGCCATCGAAAG |
| | | CGACATCAAGTGGCTGGGCTTCGAATGGTCCGGTGAAGTGCGCTATG |
| | | CATCCAAGTATTTCGACCAGCTGTTCGACTGGGCCGTCGAGTTGATCA |
| | | AGGCCGGCAAGGCCTACGTTGACGACCTGACCCCCGAGCAAGCCAAG |
| | | GAATACCGTGGCAGCCTGACCGAGCCGGGCAAGAACAGCCCGTTCCG |
| | | CGACCGTTCGGTCGAAGAGAACCTCGACTGGTTCAACCGCATGCGCG |
| | | CCGGTGAGTTCCCGGACGGCGCCCGCGTGCTGCGCGCCAAGATCGAC |
| | | ATGGCCTCGCCGAACATGAACCTGCGCGACCCGATCATGTACCGCAT |
| | | TCGCCATGCCCATCACCACCAGACCGGTGACAAGTGGTGCATCTACC |
| | | CCAACTACGACTTCACCCACGGTCAGTCGGACGCCATCGAAGGCATC |
| | | ACCCACTCCATCTGCACCCTGGAGTTCGAAAGCCATCGCCCTCTGTAC |
| | | GAATGGTTCCTGGACAGCCTGCCGGTGCCGGCGCACCCGCGTCAGTA |
| | | CGAATTCAGCCGCCTGAACCTGAACTACACCATCACCAGCAAGCGCA |
| | | AGCTCAAGCAACTGGTCGATGAAAAGCACGTGCATGGCTGGGACGAC |
| | | CCGCGCATGTCGACGCTCTCGGGTTTCCGTCGTCGTGGCTACACCCCG |
| | | GCGTCGATCCGCAATTTCTGCGACATGGTCGGCACCAACCGTTCTGAC |
| | | GGTGTGGTCGATTACGGCATGCTTGAGTTCAGCATCCGTCAGGATCTG |
| | | GACGCGAACGCGCCGCGCGCCATGTGCGTGCTGCGTCCGTTGAAAGT |
| | | CGTGATCACCAACTACCCGGAAGACAAGGTCGACCACCTTGAGCTGC |
| | | CGCGTCACCCGCAGAAAGAAGAGCTGGGCGTGCGCAAGCTGCCGTTC |
| | | GCGCGCGAAATCTACATCGACCGTGACGACTTCATGGAAGAGCCGCC |
| | | GAAGGGTTACAAGCGCCTGGAGCCGAACGGCGAAGTGCGCCTGCGTG |
| | | GCAGCTACGTGATCCGCGCCGACGAAGCAATCAAGGACGCCGAAGGC |
| | | AACATCGTCGAACTGCGCTGCTCGTACGATCCGGAAACACTCGGCAA |
| | | GAACCCTGAAGGCCGTAAGGTCAAGGGCGTGATCCACTGGGTGCCGG |
| | | CCGCTGCCAGCATCGAGTGCGAAGTGCGTCTGTACGATCGTCTGTTCC |
| | | GATCGCCGAACCCGGAGAAGGCCGAAGACAGCGCCAGCTTCCTGGAC |
| | | AACATCAACCCTGACTCGCTGCAAGTGCTTACAGGTTGTCGTGCTGAG |
| | | CCATCGCTTGGCGACGCACAGCCGGAAGACCGTTTCCAGTTCGAGCG |
| | | CGAAGGTTACTTCTGCGCGGATATCAAGGACTCGAAACCCGGTGCTC |
| | | CGGTATTCAACCGTACCGTGACCTTGCGTGATTCGTGGGGCCAGTGA |
| 89 | DP1 DNA gyrase | ATGAGCGAAGAAAACACGTACGACTCGACCAGCATTAAAGTGCTGAA |
| | subunit B | AGGTTTGGATGCCGTACGCAAACGTCCCGGTATGTACATCGGCGACA |
| | | CCGATGATGGTAGCGGTCTGCACCACATGGTGTTCGAGGTGGTCGAC |
| | | AACTCCATCGACGAAGCTTTGGCCGGTCACTGCGACGACATCAGCAT |
| | | TATCATCCACCCGGATGAGTCCATCACGGTGCGCGACAACGGTCGCG |
| | | GCATTCCGGTCGATGTGCACAAAGAAGAAGGCGTTTCGGCGGCTGAG |
| | | GTCATCATGACCGTGCTGCACGCCGGCGGTAAGTTCGATGACAACTCT |
| | | TATAAAGTCTCCGGCGGTCTGCACGGTGTAGGTGTGTCGGTAGTGAA |
| | | CGCACTGTCCGAAGAGCTGATCCTGACCGTTCGCCGTAGCGGCAAGA |
| | | TTTGGGAGCAGACGTACGTCCATGGTGTGCCACAAGAGCCGATGAAA |
| | | ATCGTTGGCGACAGTGAATCCACGGGTACGCAGATCCACTTCAAGCC |
| | | ATCGGCTGAAACCTTCAAGAACATCCACTTTAGCTGGGACATCCTGGC |
| | | CAAGCGGATTCGCGAACTGTCCTTCCTCAACTCCGGTGTGGGTATCGT |
| | | CCTCAAGGACGAGCGCAGCGGCAAGGAAGAACTGTTCAAGTACGAA |
| | | GGCGGTCTGCGCGCGTTCGTTGAATACCTGAACACCAATAAGACCGC |
| | | GGTCAACCAGGTGTTCCACTTCAACATTCAGCGTGAAGACGGCATCG |
| | | GCGTGGAAATCGCCCTGCAGTGGAACGACAGCTTCAACGAGAACTTG |
| | | TTGTGCTTCACCAACAACATTCCACAGCGCGATGGCGGTACTCACTTG |
| | | GTGGGTTTCCGTTCCGCACTGACGCGTAACCTGAACACTTACATCGAA |
| | | GCCGAAGGCTTGGCCAAGAAGCACAAAGTCGCCACCACCGGTGACGA |
| | | TGCGCGTGAAGGCCTGACCGCGATTATCTCGGTGAAAGTGCCGGATC |
| | | CCAAGTTCAGCTCCCAGACCAAAGACAAGCTGGTTTCTTCCGAGGTG |
| | | AAGACCGCCGTGGAACAGGAGATGGGCAAGTACTTCTCCGACTTCCT |
| | | GCTGGAGAACCCGAACGAAGCCAAGCTGGTCGTCGGCAAGATGATCG |
| | | ACGCTGCACGTGCTCGCGAAGCGGCGCGTAAAGCCCGTGAGATGACC |
| | | CGTCGTAAAGGCGCGCTGGATATTGCTGGCTTGCCTGGCAAGTTGGCT |
| | | GACTGCCAGGAGAAGGACCCAGCGCTCTCCGAGCTATATCTTGTGGA |
| | | AGGTGACTCTGCTGGCGGTTCCGCCAAGCAGGGTCGTAACCGTCGCA |
| | | CCCAGGCGATCCTGCCGTTGAAAGGCAAGATTCTCAACGTAGAGAAG |
| | | GCCCGCTTCGACAAGATGATTTCCTCCCAGGAAGTCGGCACCTTGATT |
| | | ACGGCGTTGGGTTGCGGCATTGGCCGCGATGAGTACAACATCGACAA |
| | | GCTGCGCTACCACAACATCATCATCATGACCGATGCTGACGTCGACG |
| | | GTTCGCACATCCGTACCTTGCTGCTGACCTTCTTCTTCCGTCAGTTGCC |
| | | TGAGCTGATTGAGCGTGGCTACATCTATATCGCGCAGCCGCCGTTGTA |
| | | CAAAGTGAAAAAGGGCAAGCAAGAGCAGTACATCAAAGACGACGAC |
| | | GCCATGGAAGAGTACATGACGCAGTCGGCCCTGGAAGATGCAAGCCT |
| | | GCACTTGAACGACGAAGCACCGGGTATCTCCGGTGAGGCGTTGGAGC |
| | | GTCTGGTTAACGACTTCCGTATGGTGATGAAGACCCTCAAGCGTCTAT |
| | | CGCGTCTGTACCCTCAGGAACTGACCGAGCACTTCATCTACCTGCCGG |
| | | CCGTCAGTCTGGAGCAGTTGGGTGATCATGCAGCGATGCAAGAGTGG |
| | | CTGGCTCAGTACGAAGTACGCCTGCGCACTGTTGAGAAGTCTGGCCT |
| | | GGTGTACAAAGCCAGTCTGCGTGAAGACCGTGAACGTAACGTGTGGC |
| | | TGCCGGAGGTTGAGTTGATCTCCCACGGCCTGTCGAATTACGTCACCT |
| | | TCAACCGCGACTTCTTCGGCAGTAATGACTACAAGACGGTCGTGACC |
| | | CTCGGCGCGCAGTTGAGCACCTTGCTGGATGATGGTGCTTACATTCAA |
| | | CGTGGCGAGCGTAAGAAAGCGGTCAAGGAGTTCAAGGAAGCCTTGG |
| | | ACTGGCTGATGGCGGAAAGCACCAAGCGTCATACCATTCAGCGATAC |
| | | AAAGGTCTGGGCGAGATGAACCCTGATCAGTTGTGGGAAACCACCAT |
| | | GGATCCAGCACAGCGTCGCATGCTGCGCGTGACCATCGAAGACGCCA |
| | | TTGGCGCAGATCAGATCTTCAACACCCTGATGGGTGATGCGGTCGAA |
| | | CCTCGCCGTGACTTCATCGAGAGCAATGCCTTGGCGGTGTCCAACCTG |
| | | GACTTCTGA |
| 90 | DP1 Isoleucine-- | ATGACCGACTATAAAGCCACGCTAAACCTTCCGGACACCGCCTTCCC |
| | tRNA ligase | AATGAAGGCCGGCCTGCCACAGCGCGAACCGCAGATCCTGCAGCGCT |
| | | GGGACAGTATTGGCCTGTACGGAAAGTTGCGCGAAATTGGCAAGGAT |
| | | CGTCCGAAGTTCGTCCTGCACGACGGCCCTCCTTATGCCAACGGCACG |
| | | ATTCACATCGGTCATGCGCTGAACAAAATTCTCAAGGACATGATCCTG |
| | | CGCTCGAAAACCCTGTCGGGTTTTGACGCGCCGTATGTCCCGGGCTGG |
| | | GACTGCCATGGCCTGCCGATCGAACACAAAGTCGAAGTGACCTACGG |
| | | CAAAAACCTGGGCGCGGATAAAACCCGCGAACTGTGCCGTGCCTACG |
| | | CCACTGAGCAGATCGAAGGGCAGAAGTCCGAATTCATCCGCCTGGGC |
| | | GTGCTGGGCGAGTGGGACAACCCGTACAAGACCATGAACTTCAAGAA |
| | | CGAGGCCGGTGAAATCCGTGCCTTGGCTGAAATCGTCAAAGGCGGTT |
| | | TTGTGTTCAAGGGCCTCAAGCCCGTGAACTGGTGCTTCGACTGCGGTT |
| | | CGGCCCTGGCTGAGGCGGAAGTCGAATACGAAGACAAGAAGTCCTCG |
| | | ACCATCGACGTGGCCTTCCCGATCGCCGACGACGCCAAGTTGGCCCA |
| | | GGCTTTCGGCCTGGCAAGCCTGAGCAAGCCGGCGGCCATCGTGATCT |
| | | GGACCACCACCCCGTGGACCATCCCGGCCAACCAGGCGCTGAACGTG |
| | | CACCCGGAATTCACCTACGCCCTGGTGGACGTCGGTGATCGCCTGCTG |
| | | GTGCTGGCCGAGGAAATGGTCGAGGCCTGTCTGGCGCGCTACGAACT |
| | | GCAAGGTTCGGTGATCGCCACCACCACCGGCTCCGCGCTGGAACTGA |
| | | TCAACTTCCGTCACCCGTTCTATGACCGCCTGTCGCCGGTTTACCTGG |
| | | CTGACTACGTCGAACTGGGTTCGGGTACGGGTGTGGTTCACTCCGCAC |
| | | CGGCCTACGGCGTTGACGACTTCGTGACCTGCAAAGCCTACGGTATG |
| | | GTCAACGATGACATCCTCAACCCGGTGCAGAGCAATGGTGTGTACGC |
| | | GCCATCGCTGGAGTTCTTCGGCGGCCAGTTCATCTTCAAGGCTAACGA |
| | | GCCGATCATCGACAAACTGCGTGAAGTCGGTGCGCTGCTGCACACCG |
| | | AAACCATCAAGCACAGCTACATGCACTGCTGGCGCCACAAAACCCCG |
| | | CTGATCTACCGCGCCACCGCGCAGTGGTTTATCGGCATGGACAAAGA |
| | | GCCGACCAGCGGCGACACCCTGCGTGTGCGCTCGCTCAAAGCCATCG |
| | | AAGACACCAAGTTCGTCCCGGCCTGGGGCCAGGCGCGCCTGCACTCG |
| | | ATGATCGCCAATCGTCCGGACTGGTGCATCTCCCGCCAGCGTAACTGG |
| | | GGCGTACCGATCCCGTTCTTCCTGAACAAGGAAAGCGGCGAGCTGCA |
| | | CCCACGCACCGTCGAGCTGATGGAAGCCGTGGCCTTGCGCGTTGAAC |
| | | AGGAAGGCATCGAAGCCTGGTTCAAGCTGGACGCCGCCGAGCTGCTG |
| | | GGCGACGAAGCGCCGCTGTACGACAAGAAGGCTCGGACCAACACCGT |
| | | GGCTGGTTCCACTCGTCGCTGCTGA |
| 91 | DP1 NADH- | ATGACTACAGGCAGTGCTCTGTACATCCCGCCTTATAAGGCAGACGA |
| | quinone | CCAGGATGTGGTTGTCGAACTCAATAACCGTTTTGGCCCTGACGCCTT |
| | oxidoreductase | TACCGCCCAGGCCACACGTACCGGCATGCCGGTGCTGTGGGTGGCGC |
| | subunit C/D | GCGCCAGGCTCGTCGAAGTCCTGACCTTCCTGCGCAACCTGCCCAAGC |
| | | CGTACGTCATGCTCTATGACCTGCATGGCGTGGACGAGCGTCTGCGG |
| | | ACCAAGCGCCAGGGCCTGCCGAGCGGCGCCGATTTCACCGTGTTCTA |
| | | TCACCTGCTGTCGATCGAACGTAACAGCGACGTGATGATCAAGGTCG |
| | | CCCTCTCCGAAAGCGACCTGAGCGTCCCGACCGTGACCGGCATCTGG |
| | | CCCAACGCCAGTTGGTACGAGCGTGAAGTCTGGGACATGTTCGGTAT |
| | | CGACTTCCCTGGCCACCCGCACCTGACGCGCATCATGATGCCGCCGA |
| | | CCTGGGAAGGTCACCCGCTGCGCAAGGACTTCCCTGCGCGCGCCACC |
| | | GAATTCGACCCGTTCAGCCTGAACCTCGCCAAGCAACAGCTTGAAGA |
| | | AGAGGCTGCACGCTTCCGGCCGGAAGACTGGGGCATGAAACGCTCCG |
| | | GCACCAACGAGGACTACATGTTCCTCAACCTGGGCCCGAACCACCCT |
| | | TCGGCGCACGGTGCCTTCCGTATCATCCTGCAACTGGACGGCGAAGA |
| | | AATCGTCGACTGCGTGCCGGACATCGGTTACCACCACCGTGGTGCCG |
| | | AGAAGATGGCCGAGCGCCAGTCGTGGCACAGCTTCATCCCGTACACC |
| | | GACCGTATCGACTACCTCGGCGGCGTGATGAACAATCTGCCGTACGT |
| | | GCTCTCGGTCGAGAAGCTGGCCGGTATCAAGGTGCCGGACCGCGTCG |
| | | ACACCATCCGCATCATGATGGCCGAGTTCTTCCGGATCACCAGCCACC |
| | | TGCTGTTCCTGGGTACCTACATCCAGGACGTCGGCGCCATGACCCCGG |
| | | TGTTCTTCACCTTCACCGACCGTCAGCGCGCCTACAAGGTCATCGAAG |
| | | CCATCACCGGCTTCCGCCTGCACCCGGCCTGGTACCGCATCGGCGGTG |
| | | TCGCGCACGACCTGCCAAATGGCTGGGAACGCCTGGTCAAGGAATTC |
| | | ATCGACTGGATGCCCAAGCGTCTGGACGAGTACCAGAAAGCCGCCCT |
| | | GGACAACAGCATCCTCAAGGGCCGGACCATTGGGGTCGCGGCCTACA |
| | | ACACCAAAGAGGCCCTGGAATGGGGCGTCACCGGTGCTGGCCTGCGT |
| | | TCCACCGGTTGCGATTTCGACCTGCGTAAAGCGCGCCCGTACTCCGGC |
| | | TACGAGAACTTCGAATTCGAAGTGCCGTTGGCGGCCAATGGCGATGC |
| | | CTACGACCGTTGCATCGTGCGCGTCGAAGAAATGCGCCAGAGCCTGA |
| | | AGATCATCGAGCAATGCATGCGCAACATCCGGCAGGCCCGTACAAGG |
| | | CGGACCACCCGCTGACCACGCCGCCGCCGAAAGAGCGCACGCTGCAA |
| | | CACATCGAAACCCTGATCACGCACTTCCTGCAGGTTTCGTGGGGCCCG |
| | | GTGATGCCGGCCAACGAATCCTTCCAGATGATCGAAGCGACCAAGGG |
| | | TATCAACAGTTATTACCTGACGAGCGATGGCGGCACCATGAGCTACC |
| | | GCACCCGGATTCGCACTCCAAGCTTCCCGCACCTGCAGCAGATCCCTT |
| | | CGGTGATCAAAGGTGAAATGGTCGCGGACTTGATTGCGTACCTGGGT |
| | | AGTATCGATTTCGTTATGGCCGACGTGGACCGCTAA |
| 92 | DP1 Protein | ATGGACGACAACAAGAAGAAAGCCTTGGCTGCGGCCCTGGGTCAGAT |
| | RecA | CGAACGTCAATTCGGCAAGGGTGCCGTAATGCGTATGGGCGATCACG |
| | | ACCGTCAGGCGATCCCGGCTATTTCCACTGGCTCTCTGGGTCTGGACA |
| | | TCGCACTCGGCATTGGCGGCCTGCCAAAAGGCCGTATCGTTGAAATCT |
| | | ACGGCCCTGAATCTTCCGGTAAAACCACCCTGACCCTGTCGGTGATTG |
| | | CCCAGGCGCAAAAAATGGGCGCCACTTGTGCGTTCGTCGATGCCGAG |
| | | CACGCTCTTGACCCTGAATACGCCGGCAAGCTGGGCGTCAACGTTGA |
| | | CGACCTGCTGGTTTCCCAACCGGACACCGGTGAGCAAGCCTTGGAAA |
| | | TCACCGACATGCTGGTGCGCTCCAACGCCATCGACGTGATCGTGGTCG |
| | | ACTCCGTGGCTGCCCTGGTGCCGAAAGCTGAAATCGAAGGCGAAATG |
| | | GGCGACATGCACGTGGGCCTGCAAGCCCGTCTGATGTCCCAGGCGCT |
| | | GCGTAAAATCACCGGTAACATCAAGAACGCCAACTGCCTGGTGATCT |
| | | TCATCAACCAGATCCGTATGAAGATTGGCGTGATGTTCGGCAGCCCG |
| | | GAAACCACCACCGGTGGTAACGCGTTGAAGTTCTACGCTTCGGTCCGT |
| | | CTGGATATCCGCCGTACTGGCGCGGTGAAGGAAGGCGACGAGGTGGT |
| | | GGGTAGCGAAACCCGCGTTAAAGTTGTGAAGAACAAGGTGGCCCCGC |
| | | CATTCCGTCAGGCTGAGTTCCAGATTCTCTACGGCAAGGGTATCTACC |
| | | TGAACGGCGAGATGATCGACCTGGGCGTACTGCACGGTTTCGTCGAG |
| | | AAGTCCGGTGCCTGGTATGCCTACAACGGCAGCAAGATCGGTCAGGG |
| | | CAAGGCCAACTCGGCCAAGTTCCTGGCGGACAACCCGGATATCGCTG |
| | | CCACGCTTGAGAAGCAGATTCGCGACAAGCTGCTGACCCCGGCACCA |
| | | GACGTGAAAGCTGCTGCCAACCGCGAGCCGGTTGAAGAAGTAGAAG |
| | | AAGTCGACACTGACATCTGA |
| 93 | DP1 RNA | ATGGAAATCACCCGCAAGGCTCTGAAAAAGCACGGTCGCGGCAACAA |
| | polymerase sigma | GCTGGCAATTGCCGAGCTGGTGGCCCTGGCTGAGCTGTTCATGCCAAT |
| | factor RpoD | CAAGCTGGTGCCGAAGCAATTTGAAGGCCTGGTTGAGCGTGTGCGCA |
| | | GTGCTCTTGAGCGTCTGCGTGCCCAAGAGCGCGCAATCATGCAGCTCT |
| | | GCGTACGTGATGCACGCATGCCGCGTGCCGACTTCCTGCGCCAGTTCC |
| | | CGGGCAACGAAGTGGATGAAAGCTGGACCGACGCACTGGCCAAAGG |
| | | CAAGGCGAAGTACGCCGAAGCCATTGGTCGCCTGCAGCCGGACATCA |
| | | TCCGTTGCCAGCAGAAGCTGACCGCGCTTCAAACCGAAACCGGTCTG |
| | | ACGATTGCTGAGATCAAGGACATCAACCGTCGCATGTCGATCGGTGA |
| | | GGCCAAGGCCCGCCGCGCGAAGAAAGAGATGGTTGAAGCGAACTTG |
| | | CGTCTGGTGATCTCCATCGCCAAGAAGTACACCAACCGTGGCCTGCA |
| | | ATTCCTCGATCTGATCCAGGAAGGCAACATCGGCTTGATGAAGGCTG |
| | | TGGACAAGTTCGAATACCGTCGCGGCTACAAGTTCTCGACTTATGCCA |
| | | CCTGGTGGATCCGTCAGGCGATCACTCGCTCGATCGCAGACCAGGCC |
| | | CGCACCATCCGTATTCCGGTGCACATGATCGAGACCATCAACAAGCT |
| | | CAACCGTATTTCCCGGCAGATGTTGCAGGAAATGGGTCGCGAACCGA |
| | | CGCCGGAAGAGCTGGGCGAACGCATGGAAATGCCTGAGGATAAAAT |
| | | CCGTAAGGTATTGAAGATCGCTAAAGAGCCGATCTCCATGGAAACGC |
| | | CGATTGGTGATGACGAAGACTCCCATCTGGGTGACTTCATCGAAGAC |
| | | TCGACCATGCAGTCGCCCATCGATGTGGCTACCGTTGAGAGCCTTAAA |
| | | GAAGCGACTCGCGACGTACTGTCCGGCCTCACTGCCCGTGAAGCCAA |
| | | GGTACTGCGCATGCGTTTCGGCATCGACATGAATACCGACCACACCCT |
| | | TGAGGAAGTCGGTAAGCAGTTTGACGTGACCCGTGAACGGATCCGTC |
| | | AGATCGAAGCCAAGGCACTGCGCAAGTTGCGCCACCCGACGCGAAGC |
| | | GAGCATCTACGCTCCTTCCTCGACGAGTGA |
| 94 | DP1 DNA- | ATGGCTTACTCATATACTGAGAAAAAACGTATCCGCAAGGACTTTAG |
| | directed RNA | CAAGTTGCCGGACGTCATGGATGTCCCGTACCTTCTGGCTATCCAGCT |
| | polymerase | GGATTCGTATCGTGAATTCTTGCAAGCGGGAGCGACTAAAGATCAGT |
| | subunit beta | TCCGCGACGTGGGCCTGCATGCGGCCTTCAAATCCGTTTTCCCGATCA |
| | | TCAGCTACTCCGGCAATGCTGCGCTGGAGTACGTGGGTTATCGCCTGG |
| | | GCGAACCGGCATTTGATGTCAAAGAATGCGTGTTGCGCGGTGTTACG |
| | | TACGCCGTACCTTTGCGGGTAAAAGTCCGTCTGATCATTTTCGACAAA |
| | | GAATCGTCGAACAAAGCGATCAAGGACATCAAAGAGCAAGAAGTCT |
| | | ACATGGGCGAAATCCCATTGATGACTGAAAACGGTACCTTCGTTATC |
| | | AACGGTACCGAGCGCGTTATCGTTTCCCAGCTGCACCGTTCCCCGGGC |
| | | GTGTTCTTCGACCACGACCGCGGCAAGACGCACAGCTCCGGTAAGCT |
| | | CCTGTACTCCGCGCGGATCATTCCGTACCGCGGCTCGTGGTTGGACTT |
| | | CGAGTTCGACCCGAAAGACTGCGTGTTCGTGCGTATCGACCGTCGTCG |
| | | TAAGCTGCCGGCCTCGGTACTGCTGCGCGCGCTCGGCTATACCACTGA |
| | | GCAAGTGCTTGATGCTTTCTACACCACCAACGTATTCAGCCTGAAGGA |
| | | TGAAACCCTCAGCCTGGAACTGATTGCTTCGCGTCTGCGTGGTGAAAT |
| | | TGCCGTCCTGGATATCCAGGATGAAAACGGCAAGGTCATCGTTGAAG |
| | | CTGGCCGCCGTATTACCGCGCGCCACATCAACCAGATCGAAAAAGCC |
| | | GGTATCAAGTCGCTGGACGTGCCGCTGGACTACGTCCTGGGTCGCAC |
| | | CACTGCCAAGGTCATCGTTCACCCGGCTACAGGCGAAATCCTGGCTG |
| | | AGTGCAACACCGAGCTGAACACCGAGATCCTGGCAAAAATCGCCAAG |
| | | GCCCAGGTTGTTCGCATCGAGACCCTGTACACCAACGACATCGACTG |
| | | CGGTCCGTTCATCTCCGACACGCTGAAGATCGACTCCACCAGCAACC |
| | | AATTGGAAGCGCTGGTCGAGATCTATCGCATGATGCGTCCTGGTGAG |
| | | CCACCGACCAAAGACGCTGCCGAGACCCTGTTCAACAACCTGTTCTTC |
| | | AGCCCTGAGCGCTATGACCTGTCTGCGGTCGGCCGGATGAAGTTCAA |
| | | CCGTCGTATCGGTCGTACCGAGATCGAAGGTTCGGGCGTGCTGTGCA |
| | | AGGAAGACATCGTCGCGGTACTGAAGACCTTGGTCGACATCCGTAAC |
| | | GGTAAAGGCATCGTCGATGACATCGACCACTTGGGTAACCGTCGTGT |
| | | TCGCTGCGTAGGCGAAATGGCCGAGAACCAGTTCCGCGTTGGCCTGG |
| | | TACGTGTTGAGCGTGCGGTCAAAGAGCGTCTGTCGATGGCTGAAAGC |
| | | GAAGGCCTGATGCCGCAAGATCTGATCAACGCCAAGCCAGTGGCTGC |
| | | GGCGGTGAAAGAGTTCTTCGGTTCCAGCCAGCTCTCGCAGTTCATGGA |
| | | CCAGAACAACCCGCTCTCCGAGATCACCCACAAGCGCCGTGTTTCCG |
| | | CACTGGGCCCGGGCGGTCTGACCCGTGAGCGTGCAGGCTTTGAAGTT |
| | | CGTGACGTACACCCAACGCACTACGGTCGTGTTTGCCCGATCGAAAC |
| | | GCCGGAAGGTCCGAACATCGGTCTGATCAACTCCCTTGCCGCTTATGC |
| | | ACGCACTAACCAGTACGGCTTCCTCGAGAGCCCGTACCGTGTAGTGA |
| | | AAGATGCACTGGTCACCGACGAGATCGTGTTCCTGTCCGCCATCGAA |
| | | GAAGCCGATCACGTGATCGCTCAGGCTTCGGCCACGATGAACGACAA |
| | | GAAAGTCCTGATCGACGAGCTGGTAGCTGTTCGTCACTTGAACGAGTT |
| | | CACCGTTAAGGCGCCGGAAGACGTCACCTTGATGGACGTTTCGCCGA |
| | | AGCAGGTAGTTTCGGTTGCAGCGTCGCTGATCCCGTTCCTGGAGCACG |
| | | ATGACGCCAACCGTGCGTTGATGGGTTCCAACATGCAGCGTCAAGCT |
| | | GTACCCACCCTGCGTGCCGACAAGCCGCTGGTAGGTACCGGCATGGA |
| | | GCGTAACGTAGCCCGTGACTCCGGCGTTTGCGTCGTGGCTCGTCGTGG |
| | | CGGCGTGATCGACTCTGTTGATGCCAGCCGTATCGTGGTTCGTGTTGC |
| | | CGATGACGAAGTTGAGACTGGCGAAGCCGGTGTCGACATCTACAACC |
| | | TGACCAAATACACCCGCTCGAACCAGAACACCTGCATCAACCAGCGC |
| | | CCGCTGGTGAGCAAGGGTGATCGCGTTCAGCGTAGCGACATCATGGC |
| | | CGACGGCCCGTCCACCGATATGGGTGAGCTGGCACTGGGTCAGAACA |
| | | TGCGCATCGCGTTCATGGCATGGAACGGCTTCAACTTCGAAGACTCCA |
| | | TCTGCCTGTCCGAGCGTGTTGTTCAAGAAGACCGCTTCACCACGATCC |
| | | ACATTCAGGAGCTGACCTGTGTGGCGCGTGACACCAAGCTTGGGCCA |
| | | GAGGAAATCACTGCAGACATCCCGAACGTGGGTGAAGCTGCACTGAA |
| | | CAAACTGGACGAAGCCGGTATCGTTTACGTAGGTGCTGAAGTTGGCG |
| | | CAGGCGACATCCTGGTTGGTAAGGTCACTCCGAAAGGCGAGACCCAA |
| | | CTGACTCCGGAAGAGAAGCTGTTGCGTGCCATCTTCGGTGAAAAAGC |
| | | CAGCGACGTTAAAGACACTTCCCTGCGCGTACCTACCGGTACCAAGG |
| | | GTACTGTCATCGACGTACAGGTCTTCACCCGTGACGGCGTTGAGCGTG |
| | | ATGCTCGTGCACTGTCCATCGAGAAGACTCAACTCGACGAGATCCGC |
| | | AAGGACCTGAACGAAGAGTTCCGTATCGTTGAAGGCGCGACCTTCGA |
| | | ACGTCTGCGTTCCGCTCTGGTAGGCCACAAGGCTGAAGGCGGCGCAG |
| | | GTCTGAAGAAAGGTCAGGACATCACCGACGAAATCCTCGACGGTCTT |
| | | GAGCACGGCCAGTGGTTCAAACTGCGCATGGCTGAAGACGCTCTGAA |
| | | CGAGCAGCTCGAGAAGGCCCAGGCCTATATCGTTGATCGCCGCCGTC |
| | | TGCTGGACGACAAGTTCGAAGACAAGAAGCGCAAACTGCAGCAGGG |
| | | CGATGACCTGGCTCCAGGCGTGCTGAAAATCGTCAAGGTTTACCTGG |
| | | CAATCCGTCGCCGCATTCAGCCGGGCGACAAGATGGCCGGTCGTCAC |
| | | GGTAACAAGGGTGTGGTCTCCGTGATCATGCCGGTTGAAGACATGCC |
| | | GCACGATGCCAATGGCACCCCGGTCGACGTCGTCCTCAACCCGTTGG |
| | | GCGTACCTTCGCGTATGAACGTTGGTCAGATCCTTGAAACCCACCTGG |
| | | GCCTCGCGGCCAAAGGTCTGGGCGAGAAGATCAACCGTATGATCGAA |
| | | GAGCAGCGCAAGGTCGCAGACCTGCGTAAGTTCCTGCACGAGATCTA |
| | | CAACGAGATCGGCGGTCGCAACGAAGAGCTGGACACCTTCTCCGACC |
| | | AGGAAATCCTGGATCTGGCGAAGAACCTGCGCGGCGGCGTTCCAATG |
| | | GCTACCCCGGTATTCGACGGTGCCAAGGAAAGCGAAATCAAGGCCAT |
| | | GCTGAAACTGGCAGACCTGCCGGAAAGTGGCCAGATGCAGCTGTTCG |
| | | ACGGCCGTACCGGCAACAAGTTTGAGCGCCCGGTTACTGTTGGCTAC |
| | | ATGTACATGCTGAAGCTGAACCACTTGGTAGACGACAAGATGCACGC |
| | | TCGTTCTACCGGTTCGTACAGCCTGGTTACCCAGCAGCCGCTGGGTGG |
| | | TAAGGCTCAGTTCGGTGGTCAGCGTTTCGGGGAGATGGAGGTCTGGG |
| | | CACTGGAAGCATACGGTGCTGCTTACACTCTGCAAGAAATGCTCACA |
| | | GTGAAGTCGGACGATGTGAACGGTCGGACCAAGATGTACAAAAACAT |
| | | CGTGGACGGCGATCACCGTATGGAGCCGGGCATGCCCGAGTCCTTCA |
| | | ACGTGTTGATCAAAGAAATTCGTTCCCTCGGCATCGATATCGATCTGG |
| | | AAACCGAATAA |
| 95 | DP22 Glutamine- | ATGAGTGAGGCTGAAGCCCGCCCAACAAATTTTATCCGTCAGATTATT |
| | tRNA ligase | GATGAAGATCTGGCGACCGGGAAACACAATACCGTTCATACCCGTTT |
| | | CCCGCCTGAGCCAAATGGCTATCTGCATATCGGTCATGCGAAATCTAT |
| | | CTGCCTGAACTTCGGCATTGCGCAAGACTATCAGGGGCAGTGCAACC |
| | | TGCGTTTTGACGATACCAACCCGGCAAAAGAAGACATCGAATTCGTT |
| | | GAGTCGATCAAACACGACGTCCAGTGGTTAGGTTTCGACTGGAGCGG |
| | | TGATATTCACTACTCTTCAGACTATTTTGATCAACTGCACGCTTATGC |
| | | GCTGGAACTGATCAACAAAGGTCTGGCGTACGTTGACGAACTGTCAC |
| | | CGGATCAGATCCGTGAATACCGCGGCTCGCTGACGTCTCCGGGCAAA |
| | | AACAGCCCGTACCGTGACCGTTCAGTGGAAGAGAACATCGCGCTGTT |
| | | TGAGAAAATGCGTAACGGTGAATTTGCCGAAGGCGCTGCCTGTCTGC |
| | | GTGCAAAAATCGATATGGCGTCGCCTTTCTTCGTGATGCGCGATCCGG |
| | | TTCTGTACCGTATTAAGTTTGCAGAACACCACCAGACCGGCAAAAAA |
| | | TGGTGCATCTATCCGATGTACGATTTCACCCACTGCATTTCCGATGCG |
| | | CTGGAAGGGATCACCCATTCGCTGTGTACGCTGGAATTCCAGGACAA |
| | | CCGCCGTCTGTACGACTGGGTTCTGGATAACATCTCCATTCCATGCCA |
| | | CCCGCGTCAGTACGAGTTCTCCCGTCTGAATCTCGAGTACTCCATCAT |
| | | GTCTAAGCGTAAGCTGAACCAGCTGGTGACCGAGAAGATTGTGGAAG |
| | | GCTGGGACGACCCGCGTATGCCGACTGTTTCAGGTCTGCGTCGTCGTG |
| | | GTTACACCGCCGCGTCTATCCGTGAATTCTGCCGTCGTATCGGCGTCA |
| | | CCAAGCAAGACAACAACGTCGAAATGATGGCGCTGGAATCCTGTATC |
| | | CGTGACGATCTGAACGAAAATGCACCGCGCGCCATGGCGGTGATCAA |
| | | CCCGGTTAAAGTGATCATTGAAAACTTTACCGGTGATGACGTGCAGA |
| | | GGGTGAAAATGCCGAACCACCCGAGCAAACCGGAAATGGGCACCCG |
| | | CGAAGTGCCATTTACCCGTGAGATTTATATCGATCAGGCAGATTTCCG |
| | | CGAAGAAGCGAACAAGCAATACAAGCGTCTGGTGCTCGGCAAAGAA |
| | | GTGCGTCTGCGCAATGCGTATGTGATCAAAGCAGAACGTATCGAGAA |
| | | AGATGCAGAAGGCAATATCACCACGATCTTCTGTTCTTACGATATCGA |
| | | TACACTGAGCAAAGATCCTGCCGATGGCCGCAAGGTGAAAGGCGTGA |
| | | TCCACTGGGTTTCGGCGTCAGAAGGCAAACCGGCGGAGTTCCGCCTG |
| | | TATGACCGTCTGTTCAGCGTCGCCAACCCGGGTCAGGCAGAAGATTTC |
| | | CTGACCACCATCAACCCGGAATCTCTGGTGATTTCCCACGGTTTCGTG |
| | | GAGCCATCACTGGTGGCTGCACAGGCTGAAATCAGCCTGCAGTTCGA |
| | | GCGTGAAGGTTACTTCTGCGCCGACAGCCGCTACTCAAGCGCTGAAC |
| | | ATCTGGTGTTTAACCGTACCGTTGGCCTGCGCGATACCTGGGAAAGCA |
| | | AACCCGTCGTGTAA |
| 96 | DP22 DNA | ATGTCGAATTCTTATGACTCCTCAAGTATCAAGGTATTAAAAGGGCTG |
| | gyrase subunit B | GACGCGGTGCGTAAGCGCCCCGGCATGTATATCGGCGATACCGATGA |
| | | CGGCACTGGTCTGCACCACATGGTATTCGAGGTTGTGGACAACGCTAT |
| | | CGACGAAGCCCTCGCGGGCCACTGTAAAGAGATTCAGGTCACGATCC |
| | | ATGCGGATAACTCTGTGTCCGTACAGGATGATGGTCGTGGCATTCCGA |
| | | CCGGTATTCATGAAGAAGAGGGCGTTTCTGCTGCTCAGGTCATCATGA |
| | | CCGTTCTTCACGCCGGCGGTAAATTTGACGATAACTCGTATAAAGTCT |
| | | CCGGCGGTCTGCATGGCGTGGGTGTTTCCGTCGTTAACGCCCTGTCAG |
| | | AAAAACTGGAACTGGTTATCCGCCGCGAAGGCAAAGTGCACACCCAG |
| | | ACTTACGTGCATGGCGAACCTCAGGATCCGCTGAAAGTGATTGGCGA |
| | | TACTGACGTGACCGGTACCACGGTACGTTTCTGGCCAAGCTTCAACAC |
| | | CTTCACCAATCACACTGAATTCGAGTATGACATTCTGGCGAAACGCCT |
| | | GCGTGAACTGTCATTCCTGAACTCCGGCGTGGCGATCCGCCTGCTGGA |
| | | TAAACGTGATGGTAAAAACGATCACTTCCATTATGAAGGCGGTATCA |
| | | AAGCTTTCGTGGAATATCTGAACAAAAACAAAACCCCAATCCATCCG |
| | | ACCGTATTCTATTTCTCCACGGTCAAAGATGACATTGGCGTTGAAGTG |
| | | GCGTTGCAGTGGAACGACGGTTTCCAGGAAAACATTTACTGCTTCACC |
| | | AACAACATTCCACAGCGCGATGGCGGGACTCACTTAGCCGGTTTCCG |
| | | TTCGGCAATGACCCGTACCCTGAACGCGTACATGGATAAAGAAGGCT |
| | | ACAGCAAGAAATCCAAAATCAGCGCCACCGGTGATGATGCCCGTGAA |
| | | GGCCTGATTGCTGTGGTGTCGGTGAAGGTGCCGGATCCTAAGTTCTCT |
| | | TCTCAGACCAAAGACAAACTGGTGTCTTCTGAAGTGAAAACAGCGGT |
| | | TGAAACGCTGATGAACGAGAAGCTGGTGGATTACCTGATGGAAAACC |
| | | CGTCAGACGCCAAAATCGTTGTCGGTAAAATCATCGACGCAGCGCGT |
| | | GCCCGTGAAGCAGCACGTAAAGCGCGTGAAATGACCCGCCGTAAAGG |
| | | CGCGCTGGATCTGGCTGGCTTGCCAGGCAAACTGGCGGACTGTCAGG |
| | | AACGCGATCCGGCACATTCCGAACTGTACTTAGTGGAAGGGGACTCA |
| | | GCGGGCGGCTCTGCAAAACAAGGCCGTAACCGTAAGAACCAGGCGAT |
| | | TCTGCCGTTGAAAGGTAAAATCCTCAACGTGGAGAAAGCGCGCTTCG |
| | | ACAAAATGCTCTCTTCTCAGGAAGTGGCAACGCTGATTACAGCACTC |
| | | GGTTGCGGCATTGGCCGTGACGAATACAACCCGGACAAACTGCGCTA |
| | | TCACAGCATCATCATCATGACCGATGCCGACGTCGATGGTTCGCACAT |
| | | CCGTACCCTGTTGCTGACATTCTTCTACCGTCAGATGCCTGAAATTGT |
| | | AGAACGTGGCCACGTGTTTATCGCCCAGCCGCCGTTGTACAAAGTGA |
| | | AAAAAGGCAAGCAGGAACAGTACATTAAAGATGACGAAGCGATGGA |
| | | TCAGTATCAGATTTCCATTGCGATGGACGGGGCAACGTTACACGCCA |
| | | ACGCTCATGCGCCAGCCCTGGCGGGTGAACCGCTGGAGAAACTGGTC |
| | | GCTGAACATCACAGCGTGCAGAAAATGATTGGCCGCATGGAACGTCG |
| | | TTATCCGCGTGCGCTGCTGAATAACCTGATCTATCAGCCGACCCTGCC |
| | | GGGTGCAGATCTGGCCGATCAGGCGAAAGTGCAGGCCTGGATGGAAT |
| | | CGCTGGTGGCGCGTCTCAACGAGAAAGAGCAGCACGGCAGTTCTTAC |
| | | AGCGCGATCGTGCGTGAAAACCGCGAACATCAGCTGTTCGAACCGGT |
| | | TCTGCGTATCCGCACCCACGGTGTTGATACCGATTACGATCTGGATGC |
| | | CGACTTCATCAAAGGCGGCGAATACCGCAAAATCTGTGCGCTGGGTG |
| | | AACAGCTGCGCGGCCTGATCGAAGAAGATGCCTTCATCGAACGTGGC |
| | | GAACGCCGTCAGCCCGTCACCAGCTTCGAACAGGCGCTGGAATGGCT |
| | | GGTGAAAGAGTCCCGTCGTGGTCTGTCGATTCAGCGATACAAAGGTC |
| | | TGGGTGAAATGAACCCTGAACAGCTGTGGGAAACCACCATGGATCCT |
| | | GAGCAACGTCGCATGTTACGTGTGACCGTGAAGGATGCCATCGCCGC |
| | | TGACCAGTTGTTCACGACGCTGATGGGCGATGCGGTTGAACCGCGCC |
| | | GCGCCTTTATCGAAGAGAACGCCCTGAAAGCCGCCAATATCGATATC |
| | | TGA |
| 97 | DP22 Isoleucine | ATGAGTGACTACAAGAACACCCTGAATTTGCCGGAAACAGGGTTCCC |
| | tRNA ligase | GATGCGTGGCGATCTGGCCAAGCGTGAACCTGACATGCTGAAAAATT |
| | | GGTATGACCAGGATCTGTACGGGATTATTCGTGCTGCCAAGAAAGGC |
| | | AAAAAAACCTTTATTTTGCATGACGGCCCTCCGTATGCGAACGGCAG |
| | | CATTCATATTGGTCACTCAGTAAACAAAATTCTTAAAGACATGATTAT |
| | | CAAGTCCAAAGGACTTGCGGGCTTTGATGCGCCGTATGTGCCGGGCT |
| | | GGGATTGTCATGGTCTGCCGATCGAGCTGAAAGTCGAACAACTGATC |
| | | GGTAAGCCGGGCGAGAAAGTTACGGCGGCGGAATTCCGTGAAGCCTG |
| | | CCGTAAATATGCCGCAGAACAGGTTGAAGGCCAGAAGAAAGACTTCA |
| | | TCCGTCTGGGCGTGCTGGGCGACTGGGATCATCCGTACCTGACGATG |
| | | GATTTCAAAACCGAAGCCAACATCATCCGTGCGCTGGGCAAAATCAT |
| | | CGGTAACGGCCACCTGCATAAAGGCGCCAAGCCGGTGCACTGGTGTA |
| | | CAGATTGCGGTTCGTCGCTGGCCGAAGCCGAAGTCGAATATTACGAC |
| | | AAAGCCTCGCCTTCTATTGATGTGGCGTTCAACGCGACGGATGCCGCA |
| | | GCCGTGGCAGCGAAATTTGGCGTTACTGCCTTTAATGGCCCGATCTCG |
| | | CTGGTTATCTGGACCACAACACCGTGGACTATGCCCGCTAACCGCGCC |
| | | ATTTCACTGAATCCTGAGTTTGCTTATCAGCTGGTTCAGGTCGAAGGT |
| | | CAGTGTCTGATCCTGGCAACCGATCTGGTTGAAAGCGTCATGAAACG |
| | | TGCCGGTATTGCCGGATGGACCGTTCTGGGCGAGTGCAAAGGCGCAG |
| | | ACCTCGAACTGCTGCGCTTCAAACACCCGTTCCTCGGTTTCGACGTTC |
| | | CGGCGATCCTGGGCGATCACGTGACGCTCGATGCGGGTACCGGTGCC |
| | | GTGCATACCGCACCAGGCCACGGCCCTGACGACTTTGTTATCGGCCA |
| | | GAAATACGGTCTGGAAGTGGCGAATCCGGTAGGGCCGAACGGTTGCT |
| | | ACCTGCCGGGCACTTACCCGACGCTGGACGGTAAATTTGTCTTTAAAG |
| | | CCAACGACCTGATCGTTGAGTTGCTGCGTGAAAAAGGCGCATTGCTG |
| | | CACGTTGAGAAAATCACGCACAGCTATCCTTGCTGCTGGCGCCACAA |
| | | AACGCCAATCATCTTCCGCGCGACGCCGCAATGGTTCATCAGCATGG |
| | | ATCAGAAGGGCCTGCGTCAGCAGTCGCTGGAAGAGATCAAAGGCGTG |
| | | CAGTGGATCCCGGACTGGGGTCAGGCACGTATCGAAAACATGGTCGC |
| | | TAACCGTCCTGACTGGTGTATCTCCCGTCAGCGTACCTGGGGCGTGCC |
| | | GATGTCTCTGTTCGTTCACAAAGACACTGAGCAGCTGCATCCGCGCAG |
| | | CCTTGAGCTGATGGAAGAAGTGGCGAAACGTGTTGAGGTGGATGGCA |
| | | TTCAGGCGTGGTGGGATCTGAATCCGGAAGACATTCTGGGTGCAGAC |
| | | GCCGCAGATTACGTCAAAGTACCGGACACGCTGGACGTCTGGTTTGA |
| | | CTCCGGTTCAACGCATTCTTCCGTTGTGGATGTGCGTCCTGAGTTCAA |
| | | CGGGCATTCTCCTGATCTGTATCTGGAAGGTTCTGACCAGCATCGCGG |
| | | CTGGTTCATGTCTTCCCTGATGATTTCGACGGCAATGAAAGGCAAAGC |
| | | GCCTTACAAACAAGTGCTGACTCACGGTTTCACCGTGGATGGTCAGG |
| | | GCCGCAAAATGTCTAAATCCATCGGCAATACCATCGCGCCGCAAGAC |
| | | GTGATGAACAAGCTGGGTGGCGACATTCTGCGTCTGTGGGTCGCGTC |
| | | GACGGATTACACCGGCGAAATCGCCGTGTCCGACGAAATCCTCAAAC |
| | | GTGCTGCTGATTCTTACCGCCGTATCCGTAACACCGCGCGCTTCCTGC |
| | | TGGCGAACCTTAACGGTTTCGATCCGGCGCTGCACAGCGTGGCTCCG |
| | | GAAGACATGGTGGTGCTGGACCGCTGGGCGGTTGGCCGTGCGAAAGC |
| | | CGCTCAGGAAGAAATCATTGCTGCGTATGAAGCCTATGATTTCCATGG |
| | | CGTTGTTCAGCGTCTGATGCAGTTCTGCTCGATCGAAATGGGTTCCTT |
| | | CTATCTGGATATCATTAAAGATCGTCAGTACACCGCGAAAAGCGACA |
| | | GCGTTGCACGTCGCAGCTGTCAGACCGCGCTGTATCACATCAGTGAA |
| | | GCGCTGGTTCGCTGGATGGCACCGATCATGTCGTTCACAGCCGATGA |
| | | AATCTGGGCGGAACTGCCGGGAAGCCGTGAGAAATTCGTCTTCACCG |
| | | AAGAGTGGTACGACGGTCTGTTCGGTCTCGCAGGCAACGAATCCATG |
| | | AACGATGCGTTCTGGGATGAACTGCTGAAAGTGCGTGGCGAAGTGAA |
| | | CAAAGTGATCGAACAGGCGCGTGCGGATAAACGTCTGGGCGGTTCTC |
| | | TGGAAGCAGCGGTTACGCTGTTTGCTGATGATGCGCTGGCAACAGAC |
| | | CTGCGTTCTCTGGGCAATGAACTGCGCTTTGTGCTGCTGACGTCAGGG |
| | | GCGAAAGTTGCCGCACTGAGTGATGCAGATGACGCGGCTCAGTCGAG |
| | | TGAATTGCTGAAAGGCCTGAAGATTGGTCTGGCGAAAGCAGAAGGCG |
| | | ACAAGTGCCCGCGCTGCTGGCATTACACTACCGATTAA |
| 98 | DP22 NADH- | ATGACAGATTTGACGACGCAAGATTCCGCCCTGCCAGCATGGCATAC |
| | quinone | CCGTGATCATCTCGATGATCCGGTTATCGGCGAATTGCGTAACCGTTT |
| | oxidoreductase | TGGGCCAGAGGCCTTTACTGTCCAGGCAACCCGCACCGGAATTCCCG |
| | subunit C/D | TGGTGTGGTTCAAGCGTGAACAGTTACTGGAAGCGATTACCTTTTTAC |
| | | GAAAACAGCCAAAACCTTACGTCATGCTTTTCGATTTGCATGGCTTTG |
| | | ATGAGCGTTTACGTACACACCGCGACGGTTTACCGGCTGCGGATTTTT |
| | | CCGTTTTCTACCACCTGATCTCCGTCGAGCGTAACCGCGACATCATGA |
| | | TCAAAGTGGCGTTGTCAGAAAACGATCTTCATGTTCCGACGATCACCA |
| | | AAGTGTTCCCGAACGCTAACTGGTACGAACGCGAAACATGGGAAATG |
| | | TTCGGTATTACCTTCGACGGCCATCCGCACCTGACGCGCATCATGATG |
| | | CCGCAGACCTGGGAAGGGCATCCGCTGCGTAAAGACTATCCGGCGCG |
| | | CGCCACCGAGTTCGATCCTTATGAGCTGACTAAGCAAAAAGAAGAAC |
| | | TCGAGATGGAATCGCTGACCTTCAAGCCGGAAGACTGGGGCATGAAG |
| | | CGCGGTACCGATAACGAGGACTTTATGTTCCTCAACCTCGGTCCTAAC |
| | | CACCCGTCAGCGCATGGTGCATTCCGTATTATCCTGCAGCTGGATGGC |
| | | GAAGAGATTGTCGACTGCGTGCCTGACGTCGGTTACCACCACCGTGG |
| | | TGCGGAGAAAATGGGCGAACGCCAGTCATGGCACAGCTACATTCCGT |
| | | ATACTGACCGTATCGAATATCTCGGCGGTTGTGTTAACGAAATGCCTT |
| | | ACGTGCTGGCTGTTGAAAAACTCGCCGGTATCGTGACGCCGGATCGC |
| | | GTTAACACCATCCGTGTGATGCTGTCTGAACTGTTCCGTATCAACAGC |
| | | CATCTGCTGTACATCTCTACGTTTATTCAGGACGTGGGTGCGATGACG |
| | | CCGGTATTCTTCGCCTTTACCGATCGTCAGAAAATTTACGATCTGGTG |
| | | GAAGCGATCACCGGTTTCCGTATGCACCCGGCCTGGTTCCGTATCGGT |
| | | GGCGTAGCGCATGACCTGCCGAAAGGCTGGGACCGCCTGCTGCGTGA |
| | | ATTCCTTGACTGGATGCCAGCCCGTTTGGATTCCTACGTCAAAGCGGC |
| | | GCTGAGAAACACCATTCTGATTGGCCGTTCCAAAGGCGTGGCCGCGT |
| | | ATAACGCCGACGACGCACTGGCCTGGGGCACCACCGGTGCTGGCCTG |
| | | CGCGCAACGGGTATCCCGTTCGATGTGCGTAAATGGCGTCCGTATTCA |
| | | GGTTATGAAAACTTTGACTTTGAAGTGCCGACCGGTGATGGCGTCAGT |
| | | GACTGCTATTCCCGCGTGATGCTGAAAGTGGAAGAACTTCGTCAGAG |
| | | CCTGCGCATTCTGGAACAGTGCTACAAAAACATGCCGGAAGGCCCGT |
| | | TCAAGGCGGATCACCCGCTGACCACGCCGCCACCGAAAGAGCGCACG |
| | | CTGCAACACATCGAGACCCTGATCACGCACTTCCTGCAAGTGTCGTGG |
| | | GGGCCGGTCATGCCTGCACAAGAATCTTTCCAGATGGTTGAAGCAAC |
| | | CAAAGGGATCAACAGCTACTACCTGACCAGTGACGGCAGCACCATGA |
| | | GCTACCGCACCCGTGTCCGTACGCCGAGCTTCCCGCATTTGCAGCAGA |
| | | TCCCGTCCGTAATCCGTGGCAGCCTGGTATCCGACCTGATCGTGTATC |
| | | TGGGCAGTATCGATTTTGTAATGTCAGATGTGGACCGCTAA |
| 99 | DP22 Protein | ATGGCTATTGATGAGAACAAGCAAAAAGCGTTAGCTGCAGCACTGGG |
| | RecA | CCAGATTGAAAAGCAATTCGGTAAAGGCTCCATCATGCGTCTGGGTG |
| | | AAGATCGCTCCATGGACGTTGAAACGATCTCTACCGGCTCTTTGTCTC |
| | | TGGATATCGCGTTAGGTGCCGGCGGTTTGCCAATGGGCCGTATCGTTG |
| | | AGATCTATGGCCCGGAATCTTCCGGTAAAACAACGCTGACCTTGCAA |
| | | GTTATCGCGGCTGCACAGCGTGAAGGCAAAACCTGTGCGTTCATCGA |
| | | TGCAGAACACGCCCTGGACCCGATCTACGCTAAAAAACTGGGCGTGG |
| | | ATATCGATAACCTGCTGTGTTCTCAGCCAGATACCGGCGAACAGGCTC |
| | | TGGAAATCTGTGACGCGCTGACCCGTTCAGGCGCTGTTGACGTGATCA |
| | | TCGTTGACTCCGTTGCCGCACTGACACCGAAAGCGGAAATCGAAGGC |
| | | GAAATTGGTGACTCTCACATGGGCCTCGCGGCACGTATGATGAGCCA |
| | | GGCGATGCGTAAGCTGGCCGGTAACCTGAAAAACGCCAACACCTTGC |
| | | TGATCTTCATCAACCAGATCCGTATGAAAATTGGTGTGATGTTCGGTA |
| | | ACCCGGAAACCACCACCGGCGGTAACGCCCTGAAATTCTACGCTTCT |
| | | GTGCGTCTGGATATCCGCCGTATCGGCGCGATCAAAGAAGGCGATGT |
| | | GGTTGTCGGTAGCGAAACGCGTGTGAAAGTGGTGAAGAACAAAATCG |
| | | CTGCGCCATTTAAACAAGCTGAATTCCAGATCATGTACGGCGAAGGC |
| | | ATCAATATCAACGGCGAGCTGATTGATCTCGGCGTGAAGCACAAGCT |
| | | GATCGAAAAAGCCGGTGCATGGTATAGCTACAACGGTGAGAAGATTG |
| | | GTCAGGGTAAAGCGAACTCCTGCAACTTCCTGAAAGAAAACCCGAAA |
| | | GTGGCTGCCGAGCTGGATAAAAAACTGCGTGATATGCTGTTGAGCGG |
| | | TACCGGTGAACTGAGTGCTGCGACCACGGCTGAAGATGCTGACGACA |
| | | ACATGGAAACCAGCGAAGAGTTTTAA |
| 100 | DP22 RNA | ATGGAGCAAAACCCGCAGTCACAGCTTAAGCTACTTGTCACCCGTGG |
| | polymerase sigma | TAAGGAGCAAGGCTATCTGACCTATGCTGAGGTCAATGACCATCTGC |
| | factor RpoD | CGGAAGATATCGTCGATTCCGACCAGATCGAAGACATCATCCAGATG |
| | | ATTAACGACATGGGCATCCAGGTACTTGAAGAAGCACCGGACGCCGA |
| | | TGATTTGATGCTGGCCGAAAACCGCCCTGATACCGATGAAGACGCTG |
| | | CAGAAGCCGCGGCGCAGGTGCTTTCCAGCGTTGAATCCGAAATTGGC |
| | | CGTACCACCGACCCTGTGCGTATGTATATGCGCGAGATGGGTACCGTT |
| | | GAGTTGCTGACCCGTGAAGGCGAAATCGACATCGCCAAACGTATCGA |
| | | AGACGGTATCAATCAGGTCCAGTGCTCCGTTGCTGAATATCCTGAAGC |
| | | TATCACTTATTTGTTAGAGCAATATGACCGTGTGGAAGCAGGCGAAG |
| | | TACGTCTGTCTGACCTGATCACCGGTTTTGTTGACCCGAACGCCGAAG |
| | | AAGAAATCGCACCAACTGCGACTCACGTGGGTTCTGAACTGACCACT |
| | | GAAGAGCAGAATGATGACGACGAAGACGAAGATGAAGACGACGACG |
| | | CTGAAGACGACAACAGCATCGATCCGGAACTGGCTCGCCAGAAGTTC |
| | | ACCGAACTGCGTGAACAGCATGAAGCGACGCGTCTGGTCATCAAGAA |
| | | AAACGGCCGTAGTCACAAGAGCGCAGCAGAAGAAATCCTGAAGCTGT |
| | | CCGATGTGTTCAAACAGTTCCGTCTGGTGCCAAAACAGTTCGATTTCC |
| | | TGGTTAACAGCATGCGTTCCATGATGGATCGCGTTCGTGCTCAGGAAC |
| | | GTCTGATCATGAAAGTGTGCGTTGAACAGTGCAAAATGCCGAAGAAA |
| | | AACTTCGTCAATCTGTTCGCCGGTAACGAAACCAGCGATACCTGGTTT |
| | | GATGCCGCTCTGGCAATGGGTAAACCATGGTCCGAGAAGCTGAAAGA |
| | | AGTCACCGAAGACGTGCAACGCGGCCTGATGAAACTGCGTCAGATCG |
| | | AAGAAGAAACCGGCCTGACTATCGAACAGGTTAAAGACATCAACCGT |
| | | CGCATGTCGATCGGCGAAGCGAAAGCCCGTCGCGCGAAGAAAGAGA |
| | | TGGTTGAAGCAAACTTACGTCTGGTTATTTCTATCGCCAAGAAATACA |
| | | CCAACCGTGGTCTGCAGTTCCTTGACCTGATCCAGGAAGGTAACATCG |
| | | GCCTGATGAAAGCCGTTGATAAGTTTGAATATCGCCGTGGTTATAAGT |
| | | TCTCAACTTATGCGACCTGGTGGATCCGTCAGGCTATCACCCGCTCCA |
| | | TCGCCGACCAGGCGCGTACCATCCGTATCCCGGTACATATGATTGAG |
| | | ACGATCAACAAACTCAACCGTATCTCCCGTCAGATGCTGCAAGAGAT |
| | | GGGCCGCGAACCGACACCGGAAGAGCTGGCTGAGCGTATGTTGATGC |
| | | CGGAAGACAAAATCCGCAAAGTGCTGAAAATTGCCAAAGAGCCAATC |
| | | TCCATGGAAACGCCAATCGGCGACGATGAAGATTCGCATCTGGGCGA |
| | | TTTCATCGAGGATACCACCCTCGAGCTGCCACTGGATTCTGCGACGTC |
| | | TGAAAGCCTGCGTTCTGCAACGCATGACGTTCTGGCTGGCCTGACTGC |
| | | ACGTGAAGCGAAAGTTCTGCGTATGCGTTTCGGTATCGATATGAACA |
| | | CTGACCACACGCTGGAAGAAGTGGGCAAACAGTTCGACGTGACCCGT |
| | | GAGCGTATCCGTCAGATCGAAGCGAAAGCGTTGCGTAAACTGCGCCA |
| | | CCCGAGCCGCTCCGAAGTACTGCGCAGCTTCCTGGACGATTAA |
| 101 | DP22 DNA- | GTGAAAGACTTACTAAAGTTTCTGAAAGCGCAAACTAAGACCGAAGA |
| | directed RNA | GTTTGATGCGATCAAAATTGCTCTGGCATCGCCAGACATGATCCGTTC |
| | polymerase | TTGGTCTTTTGGTGAAGTTAAGAAGCCAGAAACCATTAACTACCGTAC |
| | subunit beta′ | GTTCAAACCAGAACGTGACGGCCTTTTCTGTGCCCGTATTTTCGGACC |
| | | AGTAAAAGACTACGAATGCCTGTGCGGTAAGTACAAGCGTTTAAAAC |
| | | ATCGCGGCGTGATCTGCGAGAAGTGCGGCGTTGAAGTGACCCAGACT |
| | | AAAGTACGCCGTGAGCGTATGGGCCACATCGAACTGGCTTCCCCGAC |
| | | TGCACACATCTGGTTCCTGAAATCGCTGCCATCGCGCATCGGTTTGCT |
| | | GCTGGATATGCCACTGCGTGACATCGAACGTGTTCTGTACTTCGAATC |
| | | CTATGTGGTTATCGAAGGCGGCATGACTAACCTCGAAAAACGCCAGA |
| | | TCCTGACTGAAGAGCAGTATCTGGATGCGTTGGAAGAGTTTGGTGAT |
| | | GAGTTCGACGCGAAGATGGGTGCGGAAGCTATTCAGGCCCTGTTGAA |
| | | AAACATGGATCTGGAAGCAGAGTGCGAGCAACTGCGTGAAGAGTTGA |
| | | ACGAAACCAACTCCGAAACCAAACGTAAGAAGCTGACCAAGCGTATC |
| | | AAGCTGCTGGAAGCGTTCGTTCAGTCTGGTAACAAACCAGAGTGGAT |
| | | GATCCTGACTGTGCTGCCGGTACTGCCACCAGACTTGCGTCCATTGGT |
| | | TCCGTTGGACGGCGGCCGTTTCGCAACGTCGGATCTGAACGATCTGTA |
| | | TCGTCGCGTGATCAACCGTAACAACCGTCTGAAACGCCTGCTGGATCT |
| | | GGCTGCGCCAGACATCATCGTACGTAACGAAAAACGTATGCTGCAAG |
| | | AAGCGGTAGATGCTTTGCTGGATAACGGCCGTCGCGGTCGTGCTATC |
| | | ACCGGCTCTAACAAGCGTCCGCTGAAATCTCTGGCAGACATGATTAA |
| | | AGGTAAACAGGGTCGTTTCCGTCAGAACTTGCTGGGTAAACGTGTCG |
| | | ACTACTCTGGTCGTTCCGTTATCACCGTAGGTCCATACCTGCGTCTGC |
| | | ACCAGTGTGGTCTGCCGAAGAAAATGGCACTGGAACTGTTCAAACCG |
| | | TTCATCTACGGCAAGCTGGAACTGCGTGGCCTGGCCACCACCATCAA |
| | | AGCCGCGAAGAAAATGGTTGAGCGCGAAGAAGCTGTCGTTTGGGACA |
| | | TCCTGGACGAAGTTATCCGCGAACACCCGGTACTGCTGAACCGTGCA |
| | | CCAACCCTGCACCGTTTGGGTATCCAGGCGTTTGAACCGGTTCTGATC |
| | | GAAGGTAAAGCAATCCAGCTGCACCCGCTGGTTTGTGCGGCATATAA |
| | | CGCCGACTTCGATGGTGACCAGATGGCTGTTCACGTACCGTTGACGCT |
| | | GGAAGCCCAGCTGGAAGCGCGTGCGTTGATGATGTCTACCAACAACA |
| | | TCCTGTCACCTGCGAACGGCGAGCCAATCATCGTTCCTTCTCAGGACG |
| | | TTGTATTGGGTCTGTACTACATGACCCGTGACTGTGTTAACGCCAAAG |
| | | GCGAAGGCATGGTTCTGACCGGTCCTAAAGAAGCTGAGCGTATTTAC |
| | | CGCGCCGGTTTGGCCTCTCTGCATGCGCGTGTCAAAGTGCGTATTACA |
| | | GAAGAGATCAAAAATACCGAAGGCGAAGTTACGCACAAGACGTCGA |
| | | TTATCGACACGACAGTTGGTCGCGCCATCCTTTGGATGATCGTACCTA |
| | | AAGGTCTGCCGTTCTCTATCGTCAACCAGCCTCTGGGCAAAAAAGCTA |
| | | TCTCCAAAATGCTGAACACCTGTTACCGCATTTTGGGCCTGAAGCCGA |
| | | CCGTTATTTTTGCTGACCAGATCATGTACACCGGTTTTGCTTACGCTGC |
| | | CCGTTCAGGCGCGTCAGTAGGTATCGATGACATGGTAATCCCTGCGA |
| | | AGAAAGCAGAGATCATCGAAGAAGCAGAAACCGAAGTTGCTGAAAT |
| | | CCAGGAACAGTTCCAGTCTGGTCTGGTCACTGCTGGCGAACGCTATA |
| | | ACAAAGTGATCGACATCTGGGCTGCGGCCAACGAACGTGTTGCTAAG |
| | | GCAATGATGGAAAACTTGTCTGTTGAAGACGTCGTCAACCGTGACGG |
| | | TGTTGTTGAACAGCAGGTTTCCTTCAACAGTATCTTTATGATGGCCGA |
| | | CTCCGGTGCGCGTGGTTCTGCTGCACAGATTCGTCAGCTGGCCGGTAT |
| | | GCGTGGCCTGATGGCGAAACCAGATGGTTCCATCATTGAAACGCCAA |
| | | TCACCGCGAACTTCCGTGAAGGTCTGAACGTACTCCAGTACTTCATCT |
| | | CTACTCACGGTGCTCGTAAAGGTTTGGCGGATACCGCACTTAAAACG |
| | | GCTAACTCCGGTTATCTGACCCGTCGTCTGGTTGACGTCGCGCAGGAT |
| | | CTGGTTGTGACCGAAGACGACTGTGGGACTCACGAAGGCATCATGAT |
| | | GACTCCGGTCATCGAAGGTGGCGACGTTAAAGAACCACTGCGTGAGC |
| | | GTGTACTGGGTCGTGTGACTGCAGAAGATATCCTCAAGCCGGGTACG |
| | | GCGGATATCCTGGTTCCACGTAACACCCTGCTTCACGAGAAGACGTGT |
| | | GATCTGTTAGAAGAGAACTCAGTCGACAGCGTGAAAGTACGTTCAGT |
| | | CGTAAGTTGCGAAACCGACTTTGGTGTGTGTGCAAACTGCTACGGTCG |
| | | CGACCTGGCACGTGGTCACATCATCAACAAAGGTGAAGCGATCGGTG |
| | | TTATTGCAGCACAGTCCATCGGTGAGCCGGGTACCCAGCTGACGATG |
| | | CGTACGTTCCACATCGGTGGTGCGGCATCTCGTGCGGCAGCGGAATC |
| | | CAGCATCCAGGTTAAGAACACTGGTACCATTAAACTGAGCAACCACA |
| | | AGCACGTTAGCAACTCTAACGGCAAACTGGTGATCACTTCCCGTAAC |
| | | ACTGAGCTGAAATTGATCGACGAATTCGGTCGTACCAAAGAAAGCTA |
| | | TAAAGTGCCTTACGGTTCCGTGATGGGCAAAGGCGATGGCGCATCAG |
| | | TTAACGGCGGCGAAACCGTTGCTAACTGGGATCCGCACACCATGCCA |
| | | GTTATCAGTGAAGTGAGTGGTTTCATTCGCTTTGCCGATATGGTGGAT |
| | | ACTCAGACCATCACACGCCAGACCGACGACCTGACCGGTTTGTCTTCT |
| | | CTGGTTGTTCTGGACTCTGCAGAGCGTACCGGTAGCGGTAAAGACCT |
| | | GCGTCCGGCACTGAAAATCGTTGACGCTAAAGGCGACGACGTATTGA |
| | | TTCCAGGTACTGATATGCCTGCTCAATACTTCCTGCCAGGTAAAGCGA |
| | | TTGTTCAGCTGGAAGATGGTACTCAGATCCACTCTGGTGACACCCTGG |
| | | CGCGTATTCCTCAGGAATCCGGCGGTACCAAGGACATCACCGGTGGT |
| | | CTGCCACGCGTTGCTGACCTGTTCGAAGCACGTCGTCCGAAAGAGCCT |
| | | GCAATCCTTGCTGAAATCAGCGGGATCATCTCCTTCGGTAAAGAAAC |
| | | CAAAGGCAAACGTCGTCTGGTAATTTCTCCGTTAGATGGCAGCGATG |
| | | CTTACGAAGAAATGATCCCTAAATGGCGTCAGCTGAACGTGTTCGAA |
| | | GGCGAAGTTGTGGAACGTGGTGACGTCGTATCCGACGGCCCTGAGTC |
| | | TCCGCACGACATCTTGCGTTTACGTGGTGTTCACGCGGTTACCCGCTA |
| | | CATCACCAACGAAGTGCAGGAAGTTTACCGTCTGCAAGGCGTTAAGA |
| | | TTAACGATAAGCACATCGAAGTTATCGTTCGTCAGATGTTGCGTAAAG |
| | | GCACCATCGTTAGCGCTGGTGGCACTGACTTCCTGGAAGGCGAGCAG |
| | | GCAGAAATGTCTCGCGTTAAAATCGCTAACCGTAAGCTGGAAGCTGA |
| | | AGGCAAAATCACGGCAACATTCAGCCGTGACCTGCTCGGTATCACCA |
| | | AGGCATCCCTGGCGACCGAATCCTTCATCTCTGCAGCGTCGTTCCAGG |
| | | AAACCACGCGTGTTCTTACCGAAGCGGCTGTTGCCGGTAAACGTGAT |
| | | GAACTGCGTGGCCTGAAAGAGAACGTTATCGTTGGCCGTCTGATCCC |
| | | AGCCGGTACCGGTTACGCTTATCATCAGGATCGTGCACGCCGTAAAG |
| | | CACAAGGCGAAGTGCCAGTTGTACCGCAAGTCAGCGCGGATGAAGCA |
| | | ACGGCTAACCTGGCTGAACTGCTGAACGCAGGTTTCGGTAACAGCGA |
| | | CGATTAA |
| 102 | DP67 Glutamine- | ATGAGTGAGGCTGAAGCCCGCCCAACTAACTTTATTCGTCAGATTATC |
| | tRNA ligase | GACGAAGATCTGGCGAACGGTAAGCACAGTTCAGTGCACACCCGCTT |
| | | CCCGCCTGAGCCGAATGGCTATCTGCATATTGGCCATGCGAAATCAAT |
| | | CTGCCTGAACTTTGGTATCGCTCAGGATTATCAGGGGCAGTGTAACCT |
| | | GCGCTTTGATGACACTAACCCGGTGAAAGAAGATCTGGAGTTTGTTG |
| | | AATCAATCAAGCGTGATGTGCAGTGGCTGGGCTTTAAGTGGAGTGGT |
| | | GACGTACGCTACTCATCTGACTATTTCGAGCAACTGCACAATTATGCC |
| | | GTTGAGCTGATTAGTAAAGGGCTGGCGTACGTTGATGAACTGTCACC |
| | | GGAGCAGATCCGTGAATACCGTGGCAGCCTGACCTCAGCGGGTAAAA |
| | | ACAGCCCCTTCCGCGATCGCAGCGTGGACGAAAACCTTGCGCTCTTTG |
| | | CAAAAATGCGCGCGGGCGGCTTTGCCGAGGGCACCGCGTGTTTACGA |
| | | GCCAAAATTGATATGGCTTCCAACTTTATCGTTCTGCGCGATCCGGTG |
| | | ATCTACCGCATCAAATTTGCCGAACATCATCAGACCGGCAATAAGTG |
| | | GTGCATCTATCCGATGTATGACTTTACCCACTGCATCTCTGATGCGCT |
| | | GGAAGGCATTACTCACTCACTGTGTACGCTGGAATTCCAGGATAACC |
| | | GTCGCCTGTACGACTGGGTGCTGGATAACATCACCATTCCGGTTCATC |
| | | CGCGTCAGTATGAATTCTCTCGCCTGAATCTTGAATATGCCATCATGT |
| | | CCAAGCGTAAGTTGAGTCAGTTGGTGACCGAGAACGTGGTGGAAGGT |
| | | TGGGATGATCCCCGTATGCTGACTGTTTCGGGTTTGCGCCGCCGTGGC |
| | | TACACTGCGGAATCCATCCGTGAATTCTGCCGCCGCATTGGGGTGACC |
| | | AAGCAGGACAATATTGTTGAAATGGCCGCTCTGGAATCCTGTATCCGT |
| | | GACGACCTCAATGAGAATGCCCCGCGTGCCATGGCAGTGATGGATCC |
| | | GGTAAAAGTGGTGATAGAAAATCTGCCTGCGCATCACGATGAGGTGA |
| | | TCACCATGCCGAATCATCCGAGCAAGCCGGAAATGGGTACCCGCGAA |
| | | GTCCCGTTCAGTCGTGAGATCTACATCGATCGTGCTGACTTCCGTGAG |
| | | GAAGCAAACAAGCAGTACAAGCGGCTGGTGCTGGGCAAAGAAGTGC |
| | | GTCTGCGTAACGCTTATGTGATCAAAGCCGAGCGCGTGGCAAAGGAC |
| | | GATGAAGGCAACATTACCTGCCTGTTCTGTACCTGTGATGTGGATACT |
| | | CTGAGCAAGGATCCGGCCGACGGGCGTAAAGTGAAGGGCGTTATCCA |
| | | CTGGGTGTCAGCTGTTCATGCCCTTCCGGCAGAGTTCCGTCTGTACGA |
| | | TCGGCTGTTCAGCGTACCGAATCCGGGGGCGGCAGAAGACTTCCTGG |
| | | CCAGCATCAACCCGGAATCTCTGGTGATCCGTCAGGGCTTCGTGGAG |
| | | CCCGGGATGCAGCAGGCGGAGGCGTCAGCCCCGTATCAGTTTGAGCG |
| | | TGAAGGCTACTTCTGCGCTGACAGTGTCTACTCCAGTGCCAGCAATCT |
| | | GGTGTTCAACCGCACCGTTGGCCTGCGTGACACCTGGGCGAAAGTCG |
| | | GCGAGTAA |
| 103 | DP67 DNA | ATGTCGAATTCTTATGACTCCTCCAGTATCAAAGTTCTGAAAGGGCTC |
| | gyrase subunit B | GATGCTGTACGCAAACGCCCGGGTATGTATATCGGCGATACGGATGA |
| | | CGGTACCGGTCTGCATCACATGGTATTTGAGGTCGTGGATAACGCCAT |
| | | TGACGAAGCGCTCGCCGGTCACTGTTCCGATATTCTTGTCACTATTCA |
| | | TGCCGATAACTCTGTTTCCGTTGTGGATGATGGCCGTGGTATTCCGAC |
| | | CGGTATTCACGAAGAAGAAGGCATCTCAGCCGCTGAAGTGATCATGA |
| | | CCGTGCTGCACGCCGGCGGTAAGTTCGACGATAACTCTTATAAAGTCT |
| | | CCGGCGGCCTGCACGGCGTGGGCGTGTCAGTGGTGAACGCCCTGTCG |
| | | GAAAAACTGGAGCTGACCATTCGTCGCGAAGGGAAAGTTCACCAGCA |
| | | GACTTACGTCCACGGCGTGCCACAGGCCCCGTTGAGTGTGAGCGGTG |
| | | AAACTGACCTGACGGGAACGCGCGTGCGTTTCTGGCCCAGCCATCAG |
| | | ACGTTCACTAACGTCGTGGAGTTCGAGTACGAAATTTTGGCAAAGCG |
| | | CCTGCGTGAGCTGTCGTTCCTGAACTCCGGTGTATCAATCAAGCTGGA |
| | | AGATAAGCGCGACGGTAAAAGCGACCATTACCACTATGAAGGTGGTA |
| | | TCAAGGCGTTTGTTGAGTACCTCAACAAGAACAAAACCCCGATCCAC |
| | | CCGAATGTGTTCTATTTCTCAACCGAGAAAGACGGCATTGGTGTGGA |
| | | AGTGGCGCTGCAGTGGAACGATGGTTTCCAGGAAAATATCTACTGCT |
| | | TTACCAACAACATCCCACAGCGGGATGGGGGCACGCACCTCGTTGGT |
| | | TTCCGTACCGCGATGACCCGTACCCTGAATGCCTACATGGATAAAGA |
| | | AGGCTACAGCAAGAAAGCCAAAGTCAGCGCCACCGGTGACGACGCG |
| | | CGTGAAGGCCTGATTGCTGTGGTGTCGGTGAAAGTGCCGGATCCGAA |
| | | ATTCTCTTCACAGACCAAAGATAAACTGGTCTCTTCTGAAGTGAAAAC |
| | | CGCCGTTGAGCAGCAGATGAACGAGCTGCTGGCAGAATACCTGCTGG |
| | | AAAACCCGACCGATGCCAAAATCGTCGTCGGTAAAATCATTGATGCG |
| | | GCCCGCGCCCGTGAAGCGGCCCGTCGTGCACGTGAAATGACCCGCCG |
| | | TAAAGGCGCGCTGGATCTGGCAGGCCTGCCGGGCAAACTGGCGGACT |
| | | GCCAGGAGCGTGATCCGGCTCTGTCCGAAATTTACCTGGTGGAAGGG |
| | | GACTCTGCGGGCGGCTCTGCCAAGCAGGGACGTAACCGTAAAAACCA |
| | | GGCCATCCTGCCGCTGAAGGGTAAAATCCTCAACGTCGAGAAGGCGC |
| | | GCTTTGACAAGATGCTCGCGTCGCAGGAAGTCGCTACGCTGATCACC |
| | | GCGCTGGGCTGTGGTATCGGTCGTGATGAGTACAACCCCGACAAACT |
| | | GCGCTATCACAGCATCATTATCATGACCGATGCCGACGTGGATGGCTC |
| | | GCATATCCGTACCCTGCTGCTGACCTTCTTCTACCGTCAGATGCCAGA |
| | | AATCATTGAGCGTGGTCATGTCTATATTGCCCAGCCACCGCTGTACAA |
| | | GGTGAAAAAAGGCAAGCAGGAGCAGTATATTAAAGACGACGATGCG |
| | | ATGGATCAGTACCAGATCGCCATCGCGCTGGACGGTGCCACGCTGCA |
| | | TGCGAACGCCAGCGCCCCGGCCCTTGGCGGTAAGCCACTGGAAGATC |
| | | TGGTGTCTGAGTTCAACAGCACGCGCAAGATGATCAAGCGCATGGAG |
| | | CGCCGTTACCCGGTGGCCTTGCTGAATGCGCTGGTCTACAACCCGACC |
| | | CTGAGCGATTTGACCGCCGAAGCGCCGGTACAGAGCTGGATGGATGT |
| | | GCTGGTGAAGTATCTGAACGACAACGACCAGCACGGCAGCACCTACA |
| | | GCGGTCTGGTACGCGAAAATCTGGAGCTGCATATCTTTGAGCCGGTA |
| | | CTGCGTATCAAAACCCACGGCGTGGATACCGATTATCCGCTCGACAG |
| | | CGAGTTTATGCTCGGCGGCGAATACCGTAAGCTCTGCGCGCTGGGTG |
| | | AGAAGCTGCGTGGCCTGATCGAAGAAGACGCGTTCATCGAACGTGGT |
| | | GAGCGGCGTCAGCCGATTGCCAGCTTTGAGCAGGCGATGGAGTGGCT |
| | | GGTTAAAGAGTCACGCCGTGGCCTGACGGTTCAGCGTTATAAAGGTC |
| | | TGGGCGAGATGAACCCGGATCAGCTGTGGGAAACCACCATGGATCCG |
| | | GACAGCCGCCGTATGCTGCGCGTGACCATCAAAGATGCCGTGGCCGC |
| | | CGACCAGCTGTTCACCACCCTGATGGGGGATGCGGTAGAGCCCCGTC |
| | | GTGCCTTTATTGAAGAGAACGCCCTGCGCGCGGCAAACATCGATATC |
| | | TGA |
| 104 | DP67 Isoleucine | ATGAGTGACTATAAATCTACCCTGAATTTGCCGGAAACGGGGTTCCC |
| | 1RNA ligase | GATGCGTGGCGATCTGGCCAAACGCGAACCGGGTATGCTGCAACGTT |
| | | GGTATGATGACAAGCTGTACGGCATCATTCGCGAAGCCAAGAAAGGG |
| | | AAAAAAACCTTTATCCTGCACGATGGCCCTCCTTACGCCAACGGCAG |
| | | CATTCATATTGGTCACTCCGTTAACAAGATTCTGAAAGACATTATCGT |
| | | TAAGTCGAAAGGCATGGCGGGCTATGACTCGCCTTATGTACCGGGTT |
| | | GGGACTGCCACGGTCTGCCTATCGAGCATAAAGTTGAGCAGATGATC |
| | | GGTAAGCCGGGAGAGAAAGTCAGCGCCGCTGAGTTCCGTGCTGCCTG |
| | | CCGCAAATACGCTGCCGAGCAGGTGGAAGGGCAGAAAGCCGACTTTA |
| | | TCCGTCTGGGTGTGTTGGGTGACTGGGATCGTCCGTATCTGACAATGA |
| | | ACTTCCAGACCGAAGCCAATATTATCCGTGCGCTGGGTAAAATCATC |
| | | GGTAACGGGCACCTGCACAAAGGGGCCAAGCCGGTACACTGGTGCCT |
| | | GGACTGCCGTTCTGCCCTGGCTGAGGCGGAAGTGGAGTACTACGATA |
| | | AAACCTCTCCGTCTATCGATGTCATGTTCAATGCGACTGATAAAGAGG |
| | | GGGTACAGGCCAAATTTGCGGCAACGAATGTTGACGGCCCGATCTCG |
| | | CTGGTGATCTGGACTACCACGCCGTGGACCATGCCGGCTAACCGCGC |
| | | TATCTCACTGCATCCTGAATTCGACTACCAGCTGGTACAGATTGAAGG |
| | | CCGTGCTCTGATCCTCGCCAAAGAGATGGTTGAGAGCGTGATGCAGC |
| | | GCGTTGGTGTTGCCGCCTGGACCGTGCTGGGCGAAGCGAAAGGGGCA |
| | | GACCTGGAGCTGATGGGCTTCCAGCATCCGTTCCTCGACCATACCTCT |
| | | CCGGTTGTGCTGGGTGAGCATGTCACGCTGGAAGCCGGTACCGGTGC |
| | | GGTCCATACCGCACCAGGCCATGGCCCGGACGACTATGTTATCGGTC |
| | | AGAAATACGGTATCGAAGTGGCTAACCCGGTCGGCCCGGATGGCTGC |
| | | TACCTGCCGGGAACCTACCCGACGCTGGATGGTGTGAACGTCTTTAA |
| | | AGCCAACGATATGATCGTTGAACTGCTGCGTGAAAAGGGTGCTCTGC |
| | | TGCACGTTGAGAAACTGTTCCACAGCTATCCACACTGCTGGCGTCATA |
| | | AAACGCCCATCATCTTCCGCGCTACGCCACAGTGGTTTATCAGCATGG |
| | | ATCAGAAGGGCCTGCGTGCGCAGTCGCTGAAAGAGATCAAGGGCGTG |
| | | CAGTGGATCCCGGACTGGGGTCAGGCACGTATTGAATCGATGGTCGC |
| | | GAACCGTCCTGACTGGTGTATTTCCCGTCAGCGTACCTGGGGCGTGCC |
| | | GATGGCGCTGTTCGTCCATAAAGACACCGAACAGCTGCACCCGGATT |
| | | CGCTGGAGCTGATGGAGAAAGTGGCGAAGCGGGTTGAGCAGGACGG |
| | | CATTCAGGCATGGTGGGATCTTGATGCCCGCGACCTGATGGGCGCCG |
| | | ATGCTGACAACTACGTTAAAGTCCCGGATACCCTGGACGTCTGGTTTG |
| | | ACTCCGGTTCAACCAGCTACTCGGTCGTCGATGCCCGCCCTGAATTTG |
| | | ACGGCAATGCCCCTGACCTGTATCTGGAAGGATCGGATCAGCACCGC |
| | | GGCTGGTTTATGTCCTCACTGATGATCTCGACCGCGATGAAAGGCAA |
| | | AGCGCCTTACCGTCAGGTACTGACGCACGGCTTCACCGTCGATGGTCA |
| | | GGGCCGTAAGATGTCCAAGTCACTGGGCAATACTGTCAGCCCGCAGG |
| | | ATGTGATGAACAAACTGGGCGCCGATATTCTGCGCCTGTGGGTCGCCT |
| | | CTACGGACTACTCCGGTGAGATCGCCGTATCCGACGAGATCCTTAAA |
| | | CGCTCTGCCGACAGCTATCGCCGCATCCGTAACACCGCACGTTTCCTG |
| | | CTGGCAAACCTTGCCGGTTTTAATCCGGAAACCGATAGGGTGAAACC |
| | | GGAAGAGATGGTGGTGGTGGATCGCTGGGCCGTTGGCCGTGCGCTGG |
| | | CGGCACAGAATGATATCGTAGCCTCGTATGAAGCTTATGACTTCCATG |
| | | AAGTCGTGCAGCGTCTGATGCAGTTCTGTTCGGTTGAGATGGGCTCCT |
| | | TCTACCTGGATATCATCAAGGATCGTCAGTACACCGCGAAGGCCGAT |
| | | GGCCTGGCGCGTCGCAGCTGTCAGACGGCGCTGTGGTATATCGTGGA |
| | | AGCGCTGGTGCGCTGGATGGCACCGATTATGTCCTTCACTGCCGATGA |
| | | AATCTGGGGTTACCTGCCGGGTAAACGCAGCCAGTATGTCTTTACCGA |
| | | AGAGTGGTTTGACGGGCTGTTCAGCCTGGAGGACAATCAGCCGATGA |
| | | ACGACAGTTACTGGGCAGAACTGCTGAAAGTACGCGGTGAAGTCAAC |
| | | AAGGTGATCGAGCAGGCCCGCGCTGATAAGCGGATTGGCGGGTCTCT |
| | | GGAAGCCAGCGTGACGCTGTATGCTGACGCAGACCTGGCCGCGAAGC |
| | | TGACCAGCCTGGGTGAGGAGCTGCGCTTTGTGTTGCTGACTTCCGGGG |
| | | CGCAGGTTGCGGATTATGCGCAGGCCACCGCTGATGCACAGCAAAGC |
| | | GAAGGGGTAAAAGGTCTGAAAATTGCCCTGAGCAAAGCGGAAGGCG |
| | | AGAAGTGCCCGCGCTGCTGGCATTACACTAACGATATCGGCCAGAAT |
| | | GCTGAACACGCTGACGTGTGCGGCCGTTGTGTCACTAACGTCGCGGG |
| | | CAGCGGCGAACAGCGTAAGTTTGCATGA |
| 105 | DP67 NADH- | GTGATCGGCGAGCTGCGTAATCGTTTTGGGCCTGATGCCTTTACAGTA |
| | quinone | CAAGCGACCCGTACCGGCGTGCCGGTGGTCTGGGTAAAACGTGAGCA |
| | oxidoreductase | GTTGCTTGAGATTATTGAGTTCCTGCGCAAGCTGCCTAAACCCTATGT |
| | subunit C/D | GATGCTGTATGACCTGCATGGCATGGATGAGCGCCTGCGTACTCACC |
| | | GTGCCGGTTTACCGGCGGCGGATTTTTCCGTTTTCTATCACTTCATCTC |
| | | CATTGAACGTAACCGCGACATCATGCTCAAGGTGGCGTTGTCTGAAA |
| | | ACGATTTGAATGTGCCCACCATCACCAAAATTTTCCCGAATGCCAACT |
| | | GGTATGAGCGTGAAACCTGGGAGATGTTTGGTATCAATGTTGAAGGC |
| | | CACCCGCACCTGACGCGCATTATGATGCCGCAGAGCTGGGAAGGGCA |
| | | TCCGCTGCGCAAAGATTACCCTGCGCGTGCGACCGAGTTCGATCCGTT |
| | | TGAACTGACCAAGCAGAAAGAAGATCTGGAGATGGAATCTCTGACCT |
| | | TCAAGCCTGAAGACTGGGGCATGAAGCGTTCGACCAACAATGAGGAC |
| | | TTCATGTTCCTCAACCTGGGCCCGAACCACCCTTCTGCGCACGGCGCG |
| | | TTCCGTATCATCCTGCAACTGGACGGTGAAGAGATCGTCGACTGCGTG |
| | | CCGGATATCGGATACCACCATCGTGGTGCCGAAAAAATGGGTGAACG |
| | | CCAGTCCTGGCACAGCTACATTCCGTATACCGACCGTATTGAGTATCT |
| | | CGGCGGCTGCGTAAACGAAATGCCGTACGTGCTGGCGGTAGAAAAGC |
| | | TGGCTGGTATCAAAGTCCCTGAGCGCGTGGAAGTCATTCGCGTGATG |
| | | CTATCAGAGCTGTTCCGTATAAACAGCCACCTGCTGTACATCTCTACG |
| | | TTTATCCAGGACGTCGGTGCTATGTCCCCGGTGTTCTTTGCCTTTACTG |
| | | ACCGCCAGAAAATTTACGACGTGGTAGAAGCCATTACCGGCTTCCGT |
| | | ATGCATCCGGCCTGGTTCCGCATTGGTGGCGTGGCGCATGATCTGCCT |
| | | AAAGGCTGGGAGCGCCTGCTGCGTGAGTTCCTGGATTGGATGCCTAA |
| | | GCGTCTGAAAGCCTATGAGCAGACCGCACTGAAAAACTCCGTGCTTA |
| | | TTGCCCGTTCCAAAGGGGTTTCTGCCTATAACATGGAAGAAGCACTG |
| | | GCCTGGGGCACGACGGGGGCTGGCCTGCGTGGTACCGGTCTGGACTT |
| | | TGATGTGCGTAAATGGCGTCCATATTCCGGTTATGAAAACTTCGATTT |
| | | CGAAGTGCCAATCGGAGATGGCGTAAGCTGTGCTTACACCCGTGTCA |
| | | TGCTGAAGATGGAAGAGATGCGCCAGAGTATGCGCATCCTGGAACAG |
| | | TGCCTGAAGAACATGCCAGCAGGCCCGTTCAAGGCTGACCATCCGCT |
| | | GACCACGCCGCCGCCGAAAGAGCGCACGCTGCAGCATATCGAAACCC |
| | | TGATCACTCACTTCCTGCAGGTTTCGTGGGGCCCGGTAATGCCGGCAA |
| | | ACGAATCCTTCCAGATGATTGAAGCGACCAAAGGGATCAACAGTTAC |
| | | TACCTGACCAGTGATGGCAGCACGATGAGCTACCGCACCCGCGTGCG |
| | | TACGCCGAGCTTCCCGCATTTGCAACAGATCCCATCGGTGATCAACGG |
| | | CAGCCTGGTATCCGATCTGATCGTATACCTCGGTAGTATCGATTTTGT |
| | | TATGTCAGACGTGGACCGCTAA |
| 106 | DP67 Protein | ATGGCTATCGACGAAAACAAGCAAAAAGCACTGGCAGCAGCGCTGG |
| | RecA | GCCAGATTGAAAAGCAGTTTGGTAAAGGCTCCATCATGCGCCTGGGT |
| | | GAAGACCGCACCATGGATGTGGAAACCATCTCAACCGGTTCTTTATC |
| | | ACTGGATATCGCGCTGGGTGCCGGTGGTTTACCAATGGGCCGTATCGT |
| | | TGAAATCTATGGCCCGGAGTCTTCCGGTAAAACCACCCTGACGCTGC |
| | | AGGTTATCGCTTCTGCACAGCGTAAAGGGAAAACCTGTGCATTTATCG |
| | | ATGCCGAGCATGCTCTGGACCCGGTCTACGCTAAAAAACTGGGCGTG |
| | | GATATCGATAACTTGCTGTGTTCTCAGCCGGATACCGGTGAGCAGGC |
| | | GCTGGAAATCTGTGATGCGCTGGCCCGTTCCGGTGCGGTTGACGTCAT |
| | | CATCGTCGACTCCGTAGCGGCGTTGACACCAAAAGCAGAAATCGAAG |
| | | GTGAAATCGGTGACTCTCATATGGGCCTTGCGGCACGTATGATGAGC |
| | | CAGGCGATGCGTAAGCTGGCCGGTAACCTGAAGAACTCCGGTACGCT |
| | | GCTGATCTTTATCAACCAGATCCGTATGAAAATTGGCGTGATGTTCGG |
| | | TAACCCGGAAACCACTACCGGTGGTAACGCTCTGAAATTCTACGCTTC |
| | | TGTCCGTCTGGATATTCGCCGCATCGGCGCGATCAAAGAGGGTGATG |
| | | AAGTGGTGGGTAGCGAAACCCGCGTTAAAGTGGTGAAAAACAAAATC |
| | | GCAGCACCGTTTAAACAGGCTGAGTTCCAGATCATGTACGGCGAAGG |
| | | TATCAACGTTTACGGTGAGCTGGTCGACCTGGGCGTGAAGCACAAGC |
| | | TGATCGAAAAAGCCGGTGCCTGGTACAGCTATAACGGTGACAAGATT |
| | | GGTCAGGGTAAAGCCAACTCAGGTAACTTCCTGAAAGAGAACCCGGC |
| | | TATCGCTAACGAAATCGAAGCAAAACTGCGTGAAATGCTGTTGAACA |
| | | GCCCGGACGATAAGCCTGATTTTGTTCCGGCTCCGCATGAAGCCGATA |
| | | GTGAAGTTAACGAAGATATCTAA |
| 107 | RNA polymerase | ATGGAGCAAAACCCGCAGTCACAGCTTAAGCTACTTGTCACCCGTGG |
| | sigma factor | TAAGGAGCAAGGCTATCTGACCTATGCCGAGGTCAATGACCATCTGC |
| | RpoD | CGGAAGATATCGTCGACTCCGATCAGATTGAAGACATCATTCAGATG |
| | | ATCAACGACATGGGCATTCAGGTTGTAGAAGAAGCGCCTGATGCCGA |
| | | TGATTTGATGCTGAATGAGAACAACAACGACACGGACGAAGACGCTG |
| | | CCGAAGCGGCTGCTCAGGTATTATCCAGCGTAGAATCTGAAATCGGA |
| | | CGTACCACCGACCCGGTGCGCATGTACATGCGCGAAATGGGGACGGT |
| | | TGAACTGCTGACGCGTGAAGGCGAGATCGATATCGCCAAACGCATCG |
| | | AAGAGGGTATCAACCAGGTACAGTGTTCCGTTGCTGAATATCCTGAA |
| | | GCGATTACTTACCTGCTTGAGCAATATGACCGTGTTGAAGCGGGCGA |
| | | AGCGCGCCTGTCGGATCTGATCACCGGTTTTGTCGACCCGAATGCCGA |
| | | AGCAGAGATCGCCCCTACTGCGACTCACGTGGGTTCAGAACTTTCCGC |
| | | TGAAGAGCGTGATGACGAAGAAGAAGACGAAGAGTCTGACGACGAC |
| | | AGCTCGGATGATGACAACAGCATCGATCCGGAACTGGCGCGGGAAAA |
| | | ATTCAACGACCTGCGCGTTCAGTACGAAACCACCCGTACCGTTATCAA |
| | | AGCGAAAAGCCGCAGCCACGCTGATGCCATCGCTGAGATCCAGAATC |
| | | TGTCCGACGTGTTCAAGCAGTTCCGCCTGGTGCCGAAGCAGTTCGACT |
| | | TCCTGGTGAACAGCATGCGCACCATGATGGATCGCGTCCGTACTCAG |
| | | GAACGCCTGATCCTCAAGCTGTGCGTAGAAATCTGTAAGATGCCGAA |
| | | GAAGAACTTCATTACCCTGTTCACCGGTAATGAAACCAGCGAAACCT |
| | | GGTTCAAAGCGGCACTGGCAATGAATAAGCCGTGGTCAGAGAAGCTG |
| | | AACGATGTGTCAGATGACGTACACCGTAGCCTGATGAAGCTGCAGCA |
| | | GATCGAAACGGAAACTGGCCTGACGATTGAACAGGTAAAAGACATCA |
| | | ACCGTCGTATGTCGATCGGCGAAGCGAAAGCGCGCCGTGCGAAGAAA |
| | | GAGATGGTTGAGGCTAACCTGCGTCTGGTTATCTCTATCGCCAAGAAG |
| | | TACACCAACCGTGGCCTGCAGTTCCTGGATCTGATTCAGGAAGGTAA |
| | | CATCGGTCTGATGAAAGCGGTGGATAAGTTTGAATATCGCCGTGGTT |
| | | ATAAGTTCTCGACTTATGCCACCTGGTGGATCCGTCAGGCGATCACCC |
| | | GTTCAATCGCTGACCAGGCGCGTACCATCCGTATTCCGGTGCACATGA |
| | | TTGAGACGATTAACAAGCTCAACCGTATTTCCCGCCAGATGCTGCAA |
| | | GAGATGGGCCGTGAGCCGACGCCGGAAGAGCTGGCCGAGCGTATGCT |
| | | GATGCCGGAAGATAAGATCCGTAAGGTGCTGAAAATTGCCAAAGAGC |
| | | CGATCTCTATGGAGACGCCGATTGGTGATGATGAAGATTCACATCTG |
| | | GGTGATTTTATCGAAGACACCACGCTGGAGCTGCCGCTGGACTCCGC |
| | | GACGTCAGAGAGCCTGCGTTCTGCCACGCACGACGTGCTGGCCGGTC |
| | | TGACCGCGCGTGAAGCCAAAGTACTGCGTATGCGTTTCGGTATCGAT |
| | | ATGAATACCGACCACACGCTGGAAGAAGTGGGCAAACAGTTCGACGT |
| | | AACGCGTGAGCGTATTCGTCAGATTGAGGCGAAAGCGCTGCGTAAGC |
| | | TGCGTCACCCAAGCCGCTCTGAAGTGCTGCGCAGCTTCCTCGACGATT |
| | | AA |
| 108 | DNA-directed | ATGGTTTACTCCTATACCGAGAAAAAACGTATTCGTAAGGATTTTGGA |
| | RNA polymerase | AAGCGTCCACAAGTTCTGGACATTCCATATCTCCTTTCTATCCAGCTT |
| | subunit beta | GACTCGTTCCAGAAGTTCATCGAGCAAGATCCGGAAGGTCAATATGG |
| | | TCTGGAAGCAGCATTCCGCTCCGTATTTCCAATCCAAAGCTATAGCGG |
| | | TAATTCTGAGCTGCAGTACGTCAGCTACCGTTTAGGCGAACCCGTCTT |
| | | TGATGTGAAAGAGTGTCAGATTCGTGGCGTCACGTATTCTGCTCCTCT |
| | | GCGCGTAAAACTGCGCCTGGTGATCTACGAGCGCGAAGCGCCGGAAG |
| | | GCACCGTTAAAGACATCAAAGAACAAGAAGTTTACATGGGCGAAATT |
| | | CCGCTCATGACGGATAACGGTACCTTTGTTATCAACGGTACTGAGCGC |
| | | GTTATCGTTTCTCAGCTCCACCGTAGTCCTGGTGTCTTCTTCGACAGCG |
| | | ATAAGGGTAAAACCCACTCGTCCGGTAAAGTGCTGTATAACGCACGT |
| | | ATCATCCCTTACCGTGGTTCATGGCTGGACTTCGAGTTCGACCCGAAA |
| | | GACAACCTGTTCGTCCGTATTGACCGTCGCCGTAAACTGCCAGCGACC |
| | | ATCATTCTGCGCGCGTTGAATTACACCACTGAACAGATCCTCGACCTG |
| | | TTCTTCGATAAAGTGGTTTACCAAATTCGCGACAACAAGCTGCAGATG |
| | | GAGCTTATTCCTGAGCGCCTGCGTGGTGAGACCGCTTCATTTGATATT |
| | | GAAGCGAACGGCACCGTTTACGTCGAAAAAGGCCGCCGTATTACTGC |
| | | GCGCCATATTCGCCAGCTTGAGAAAGATGCTGTTGCCCACATCGAAG |
| | | TGCCGGTTGAGTATATTGCCGGTAAAGTGGTCGCTAAAGACTACGTTG |
| | | ATGAGAGCACCGGTGAACTGCTGATCGCAGCGAACATGGAACTGTCA |
| | | CTGGATCTGCTGGCTAAACTCAGCCAGTCCGGTCACAAGCGCATTGA |
| | | AACCCTGTTCACCAACGATCTGGATCACGGTGCGTACATGTCTGAGAC |
| | | GGTACGTGTCGACCCAACCAGCGATCGCCTGAGCGCTCTGGTTGAGA |
| | | TCTACCGCATGATGCGTCCTGGTGAGCCACCAACGCGTGAAGCGGCT |
| | | GAAAACCTGTTTGAGAACCTGTTCTTCTCTGAAGACCGCTATGATCTG |
| | | TCTGCGGTTGGTCGTATGAAGTTCAACCGTTCTCTGCTGCGCGACGAG |
| | | ATCGAAGGTTCCGGTATCCTGAGCAAAGACGACATCATTCAGGTGAT |
| | | GAAGAAGCTCATCGGTATCCGTAACGGTATTGGCGAAGTGGATGATA |
| | | TCGACCACCTCGGCAACCGTCGTATCCGTTCCGTTGGCGAAATGGCTG |
| | | AAAACCAGTTCCGTGTTGGCCTTGTGCGCGTAGAGCGTGCGGTGAAA |
| | | GAGCGTCTGTCCCTGGGCGATCTGGATACCCTGATGCCACAGGACAT |
| | | GATCAACGCCAAGCCAATTTCTGCGGCAGTGAAAGAGTTCTTCGGCT |
| | | CCAGCCAGCTGTCACAGTTTATGGACCAGAACAACCCGTTGTCTGAG |
| | | ATCACGCATAAGCGTCGTATCTCTGCACTGGGTCCGGGCGGTCTGACG |
| | | CGTGAGCGTGCAGGCTTCGAAGTTCGAGACGTACACCCGACGCACTA |
| | | CGGTCGCGTATGTCCAATCGAAACGCCGGAAGGTCCAAACATCGGTC |
| | | TGATCAACTCCTTGTCTGTGTATGCACAGACCAATGAGTACGGTTTCC |
| | | TGGAAACCCCATACCGTCGCGTTCGCGAAGGCGTGGTGACCGACGAA |
| | | ATTCATTACCTCTCTGCTATTGAAGAGGGTAACTACGTTATCGCTCAG |
| | | GCAAACACCAATCTCGACGACGAAGGTCACTTCGTAGACGACCTGGT |
| | | CACCTGCCGTAGCAAAGGCGAATCGAGTCTCTTCAACCGCGATCAAG |
| | | TTGACTACATGGACGTTTCCACCCAGCAGGTGGTTTCCGTCGGTGCGT |
| | | CACTGATCCCGTTCCTGGAGCACGATGACGCCAACCGCGCATTGATG |
| | | GGTGCAAACATGCAACGTCAGGCGGTTCCTACTCTGCGTGCTGATAA |
| | | GCCGCTGGTAGGTACCGGTATGGAGCGTGCGGTTGCGGTTGACTCCG |
| | | GTGTTACTGCCGTAGCGAAACGTGGTGGTACCGTGCAGTACGTGGAT |
| | | GCATCCCGTATCGTTATTAAAGTTAACGAAGACGAAATGTATCCGGG |
| | | CGAAGCCGGTATCGACATTTACAACCTGACCAAATATACCCGTTCTAA |
| | | CCAGAACACCTGCATCAACCAGATGCCTTGCGTGAACCTGGGTGAGC |
| | | CAATCGAACGTGGTGATGTGCTGGCTGATGGCCCTTCAACCGATCTCG |
| | | GCGAACTGGCACTCGGTCAGAACATGCGCGTCGCGTTCATGCCGTGG |
| | | AACGGCTACAACTTCGAAGACTCCATTCTGGTCTCGGAGCGCGTTGTT |
| | | CAGGAAGATCGCTTCACCACTATCCACATTCAGGAACTGGCGTGTGT |
| | | GTCTCGTGACACCAAGCTGGGGCCAGAAGAGATCACCGCTGACATCC |
| | | CTAACGTGGGTGAAGCTGCGCTCTCTAAACTGGATGAGTCCGGTATC |
| | | GTGTATATCGGTGCGGAAGTGACCGGTGGGGACATTCTGGTTGGTAA |
| | | GGTAACACCTAAAGGTGAAACCCAGCTGACGCCAGAAGAGAAACTG |
| | | CTGCGTGCGATCTTCGGTGAAAAAGCGTCTGACGTTAAAGACTCTTCT |
| | | CTGCGCGTACCAAACGGTGTGTCAGGGACAATCATCGACGTTCAGGT |
| | | CTTTACCCGCGATGGCGTGGAAAAAGACAAGCGTGCGCTGGAAATCG |
| | | AAGAGATGCAGCTGAAGCAGGCGAAGAAAGACCTGTCTGAAGAATT |
| | | GCAGATCCTCGAAGCCGGCTTGTTCAGCCGTATTAACTACCTGCTGGT |
| | | TGCCGGCGGTGTTGAAGCGGAAAAACTGGAGAAGCTGCCACGTGAGC |
| | | GCTGGCTCGAACTGGGCCTGACCGACGAAGAGAAGCAAAATCAGCTG |
| | | GAACAGCTGGCCGAGCAGTACGACGAGCTGAAGCACGAGTTTGAGA |
| | | AAAAACTTGAAGCCAAGCGCCGTAAAATCACTCAGGGCGATGACCTG |
| | | GCACCTGGCGTGCTGAAAATCGTGAAAGTGTATCTGGCCGTTAAACG |
| | | TCAGATCCAGCCTGGTGACAAAATGGCAGGTCGTCACGGGAACAAAG |
| | | GTGTTATCTCCAAGATCAACCCGATCGAAGATATGCCATACGATGAG |
| | | TTCGGTACGCCGGTCGACATCGTACTGAACCCGCTGGGCGTTCCATCA |
| | | CGTATGAACATTGGTCAGATTCTTGAAACCCACCTGGGTATGGCTGCG |
| | | AAAGGCATTGGCGAGAAAATTAACGCTATGCTTAAGAAGCAGGAAG |
| | | AAGTGTCCAAGCTGCGTGAATTCATTCAGCGTGCTTACGATCTGGGCA |
| | | GCGATCTGCGTCAGAAAGTTGACCTGAACACCTTCACCGATGACGAA |
| | | GTGCTGCGCCTGGCAGAGAATCTGAAAAAAGGTATGCCAATTGCAAC |
| | | ACCAGTGTTTGACGGCGCGAAAGAGAGCGAAATCAAAGAGCTGTTAC |
| | | AGCTCGGCGGCCTGCCTTCTTCTGGCCAGATCACGCTGTTTGATGGTC |
| | | GTACCGGTGAGCAGTTCGAACGTCAGGTTACCGTTGGCTACATGTAC |
| | | ATGCTGAAGCTGAACCACCTGGTTGATGACAAAATGCATGCGCGTTC |
| | | TACCGGTTCTTACAGCCTCGTTACTCAGCAGCCGCTGGGTGGTAAGGC |
| | | GCAGTTCGGTGGTCAGCGCTTCGGTGAGATGGAAGTGTGGGCACTGG |
| | | AAGCATACGGTGCCGCGTATACCCTGCAGGAAATGCTGACCGTGAAG |
| | | TCTGATGACGTTAACGGCCGTACCAAGATGTATAAAAACATCGTTGA |
| | | CGGCAACCATCAGATGGAACCGGGCATGCCGGAATCTTTCAACGTAC |
| | | TGTTGAAAGAGATCCGCTCGCTGGGTATCAACATCGAGCTGGAAGAC |
| | | GAGTAA |
| 109 | DP68 Glutamine- | ATGAGCAAGCCCACTGTCGACCCTACCTCGAATTCCAAGGCCGGACC |
| | tRNA ligase | TGCCGTCCCGGTCAATTTCCTGCGCCCGATCATCCAGGCGGACCTGGA |
| | | TTCGGGCAAGCACACGCAGATCGTCACCCGCTTCCCGCCAGAGCCCA |
| | | ACGGCTACCTGCACATCGGTCACGCCAAGTCGATCTGTGTGAACTTCG |
| | | GCCTGGCCCAGGAGTTCGGTGGCGTCACGCACCTGCGTTTCGACGAC |
| | | ACCAACCCGGCCAAGGAAGACCAGGAATACATCGACGCCATCGAAA |
| | | GCGACATCAAGTGGCTGGGCTTCGAATGGTCCGGTGAAGTGCGCTAT |
| | | GCGTCCAAGTATTTCGACCAGTTGTTCGACTGGGCCGTCGAGCTGATC |
| | | AAGGCCGGCAAGGCCTACGTCGACGACCTGACCCCGGAGCAGGCCAA |
| | | GGAATACCGTGGCACGCTGACCGAGCCGGGCAAGAACAGCCCGTTCC |
| | | GTGACCGTTCGGTAGAAGAGAACCTCGACTGGTTCAACCGCATGCGC |
| | | GCCGGTGAGTTCCCGGACGGCGCCCGCGTGCTGCGCGCCAAGATCGA |
| | | CATGGCCTCGCCGAACATGAACCTGCGCGACCCGATCATGTACCGCA |
| | | TCCGCCACGCCCATCACCACCAGACCGGTGACAAGTGGTGCATCTAC |
| | | CCGAACTATGACTTCACCCACGGTCAGTCGGACGCCATCGAAGGCAT |
| | | CACCCACTCCATCTGCACCCTGGAGTTCGAAAGCCATCGCCCGCTGTA |
| | | TGAGTGGTTCCTCGACAGCCTGCCGGTTCCGGCGCACCCGCGTCAGTA |
| | | CGAGTTCAGCCGCCTGAACCTGAACTACACCATCACCAGCAAGCGCA |
| | | AGCTCAAGCAGTTGGTGGACGAAAAGCACGTGCATGGCTGGGATGAC |
| | | CCGCGCATGTCCACCCTGTCGGGTTTCCGCCGTCGCGGCTACACCCCG |
| | | GCGTCGATCCGCAGCTTCTGCGACATGGTCGGCACCAACCGCTCCGA |
| | | CGGCGTGGTCGATTACGGCATGCTCGAGTTCAGCATCCGTCAGGACCT |
| | | GGACGCCAACGCGCCGCGTGCCATGTGCGTATTGCGCCCGTTGAAAG |
| | | TCGTGATCACCAACTATCCGGAAGACAAGGTCGACCACCTCGAACTG |
| | | CCGCGTCACCCGCAGAAAGAAGAACTTGGCGTGCGCAAGCTGCCGTT |
| | | CGCGCGTGAAATCTACATCGACCGTGATGACTTCATGGAAGAGCCGC |
| | | CGAAAGGCTACAAGCGCCTGGAGCCTAACGGCGAAGTGCGCCTGCGC |
| | | GGCAGCTACGTGATCCGTGCCGATGAAGCGATCAAGGACGCCGATGG |
| | | CAACATCGTCGAACTGCGATGCTCCTACGACCCGGAAACCCTGGGCA |
| | | AGAACCCTGAAGGCCGCAAGGTCAAAGGCGTCGTTCACTGGGTGCCG |
| | | GCTGCTGCCAGCATCGAGTGCGAAGTGCGCCTGTACGATCGTCTGTTC |
| | | CGTTCGCCGAACCCTGAGAAGGCTGAAGACAGCGCCAGCTTCCTGGA |
| | | CAACATCAACCCTGACTCCCTGCAAGTTCTCACGGGTTGTCGTGCCGA |
| | | GCCATCGCTTGGCGACGCACAGCCGGAAGACCGTTTCCAGTTCGAGC |
| | | GCGAAGGTTACTTCTGCGCGGATATCAAGGACTCCAAACCTGGTCAT |
| | | CCGGTCTTCAACCGTACCGTGACCTTGCGTGATTCGTGGGGCCAGTG |
| 110 | DP68 DNA | ATGAGCGAAGAAAACACGTACGACTCGACCAGCATTAAAGTGCTGAA |
| | gyrase subunit B | AGGTTTGGATGCCGTACGCAAACGTCCCGGTATGTACATCGGCGACA |
| | | CCGATGATGGTAGCGGTCTGCACCACATGGTGTTCGAGGTGGTCGAC |
| | | AACTCCATCGACGAAGCTTTGGCCGGTCACTGCGACGACATCAGCAT |
| | | TATCATCCACCCGGATGAGTCCATCACCGTGCGCGACAACGGTCGCG |
| | | GTATTCCGGTCGATGTGCACAAAGAAGAAGGCGTATCGGCGGCAGAG |
| | | GTCATCATGACCGTGCTTCACGCCGGCGGTAAGTTCGACGACAACTCC |
| | | TATAAAGTTTCCGGCGGTTTGCACGGTGTAGGTGTGTCGGTGGTGAAC |
| | | GCTCTGTCCGAAGAGCTTATCCTGACTGTTCGCCGTAGCGGCAAGATC |
| | | TGGGAACAGACCTACGTGCATGGTGTTCCACAAGAACCGATGAAAAT |
| | | CGTTGGCGACAGTGAATCCACCGGTACGCAGATCCACTTCAAGCCTTC |
| | | GGCAGAAACCTTCAAGAATATCCACTTCAGTTGGGACATCCTGGCCA |
| | | AGCGTATTCGTGAACTGTCGTTCCTTAACTCCGGTGTGGGTATCGTCC |
| | | TCAAGGACGAGCGCAGCGGCAAGGAAGAGTTGTTCAAGTACGAAGG |
| | | CGGCTTGCGTGCGTTCGTTGAGTACCTGAACACCAACAAGACTGCGG |
| | | TCAACCAGGTGTTCCACTTCAACATCCAGCGTGAAGACGGTATCGGC |
| | | GTTGAAATCGCCCTGCAGTGGAACGACAGCTTCAACGAGAACCTGTT |
| | | GTGCTTCACCAACAACATTCCACAGCGCGACGGCGGTACTCACTTGGT |
| | | GGGTTTCCGTTCCGCACTGACGCGTAACCTGAACACCTACATCGAAGC |
| | | GGAAGGCTTGGCCAAGAAGCACAAAGTGGCCACTACCGGTGACGATG |
| | | CGCGTGAAGGCCTGACGGCGATTATCTCGGTGAAAGTGCCGGATCCA |
| | | AAGTTCAGCTCCCAGACCAAAGACAAGCTGGTGTCTTCCGAAGTGAA |
| | | GACCGCAGTGGAACAGGAGATGGGCAAGTACTTCTCCGACTTCCTGC |
| | | TGGAAAACCCGAACGAAGCCAAGTTGGTTGTCGGCAAGATGATCGAC |
| | | GCGGCGCGTGCCCGTGAAGCGGCGCGTAAAGCCCGTGAGATGACCCG |
| | | CCGTAAAGGCGCGTTGGATATCGCCGGCCTGCCGGGCAAACTGGCTG |
| | | ACTGCCAGGAGAAGGACCCTGCCCTCTCCGAACTGTACCTGGTGGAA |
| | | GGTGACTCTGCTGGCGGTTCCGCCAAGCAGGGTCGTAACCGTCGCAC |
| | | CCAGGCTATCCTGCCGTTGAAGGGTAAGATCCTCAACGTCGAGAAGG |
| | | CCCGCTTCGACAAGATGATTTCCTCTCAGGAAGTCGGCACCTTGATCA |
| | | CGGCGTTGGGCTGCGGTATTGGCCGCGATGAGTACAACATCGACAAA |
| | | CTGCGTTACCACAACATCATCATCATGACCGATGCTGACGTCGACGGT |
| | | TCGCACATCCGTACCCTGCTGCTGACCTTCTTCTTCCGTCAGTTGCCGG |
| | | AGCTGATCGAGCGTGGCTACATCTACATCGCTCAGCCGCCGTTGTACA |
| | | AAGTGAAAAAGGGCAAGCAAGAGCAGTACATCAAAGACGACGACGC |
| | | CATGGAAGAGTACATGACGCAGTCGGCCCTGGAAGATGCCAGCCTGC |
| | | ACTTGAACGACGAAGCCCCGGGCATTTCCGGTGAGGCGCTGGAGCGT |
| | | TTGGTTAACGACTTCCGCATGGTAATGAAGACCCTCAAGCGTCTGTCG |
| | | CGCCTGTACCCTCAGGAGCTGACCGAGCACTTCATCTACCTGCCTTCC |
| | | GTGAGCCTGGAGCAGTTGGGCGATCACGCCCACATGCAGAATTGGCT |
| | | GGCTCAGTACGAAGTACGTCTGCGCACCGTCGAGAAGTCTGGCCTGG |
| | | TTTACAAAGCCAGCTTGCGTGAAGACCGTGAACGTAACGTGTGGCTG |
| | | CCGGAGGTTGAACTGATCTCCCACGGCCTGTCGAACTACGTCACCTTC |
| | | AACCGCGACTTCTTCGGCAGCAACGACTACAAGACCGTGGTTACCCT |
| | | CGGCGCGCAATTGAGCACCCTGTTGGACGACGGTGCTTACATCCAGC |
| | | GTGGCGAGCGTAAGAAAGCGGTCAAGGAGTTCAAGGAAGCCCTGGA |
| | | CTGGTTGATGGCTGAAAGCACCAAGCGCCACACCATCCAGCGATACA |
| | | AAGGTCTGGGCGAGATGAACCCGGATCAACTGTGGGAAACCACCATG |
| | | GATCCTGCTCAGCGTCGCATGCTACGCGTGACCATCGAAGACGCCATT |
| | | GGCGCAGACCAGATCTTCAACACCCTGATGGGTGATGCGGTCGAGCC |
| | | TCGCCGTGACTTCATCGAGAGCAACGCCTTGGCGGTGTCTAACCTGGA |
| | | TTTCTGA |
| 111 | DP68 Isoleucine | ATGACCGACTATAAAGCCACGCTAAACCTTCCGGACACCGCCTTCCC |
| | 1RNA ligase | AATGAAGGCCGGCCTGCCACAGCGCGAACCGCAGATCCTGCAGCGCT |
| | | GGGACAGTATTGGCCTGTACGGAAAGTTGCGCGAAATTGGCAAGGAT |
| | | CGTCCGAAGTTCGTCCTGCACGACGGCCCTCCTTATGCCAACGGCACG |
| | | ATTCACATCGGTCATGCGCTGAACAAAATTCTCAAGGACATGATCCTG |
| | | CGTTCGAAAACCCTGTCGGGCTTCGACGCGCCTTATGTTCCGGGCTGG |
| | | GACTGCCACGGCCTGCCGATCGAACACAAAGTCGAAGTGACCTACGG |
| | | CAAGAACCTGGGCGCGGATAAAACCCGCGAACTGTGCCGTGCCTACG |
| | | CCACCGAGCAGATCGAAGGGCAGAAGTCCGAATTCATCCGCCTGGGC |
| | | GTGCTGGGCGAGTGGGACAACCCGTACAAGACCATGAACTTCAAGAA |
| | | CGAGGCCGGTGAAATCCGTGCCTTGGCTGAAATCGTCAAAGGCGGTT |
| | | TCGTGTTCAAGGGCCTCAAGCCCGTGAACTGGTGCTTCGACTGCGGTT |
| | | CGGCCCTGGCTGAAGCGGAAGTCGAGTACGAAGACAAGAAGTCCTCG |
| | | ACCATCGACGTGGCCTTCCCGATCGCCGACGACGACAAGCTGGCTCA |
| | | AGCCTTTGGCCTGTCCAGCCTGCCAAAGCCTGCAGCCATCGTGATCTG |
| | | GACCACCACCCCGTGGACCATCCCGGCCAACCAGGCGCTGAACGTGC |
| | | ACCCGGAATTCACCTACGCCCTGGTGGACGTCGGTGATCGCCTGCTGG |
| | | TGCTGGCTGAAGAAATGGTCGAGGCCTGCCTGGCGCGCTACGAGCTG |
| | | CAAGGTTCGGTCATCGCCACCACCACCGGCACTGCGCTGGAGCTGAT |
| | | CAATTTCCGTCACCCGTTCTATGACCGTCTGTCGCCGGTGTACCTGGC |
| | | TGACTACGTAGAGCTGGGTTCGGGTACTGGTGTGGTTCACTCCGCGCC |
| | | GGCCTACGGCGTTGATGACTTTGTGACCTGCAAAGCCTACGGCATGGT |
| | | CAACGATGACATCCTCAACCCGGTGCAGAGCAATGGCGTGTACGCGC |
| | | CGTCGCTGGAGTTCTTTGGCGGCCAGTTCATCTTCAAGGCCAACGAGC |
| | | CGATCATCGACAAACTGCGTGAAGTCGGTTCGCTGCTGCACACCGAA |
| | | ACCATCAAGCACAGCTACATGCACTGCTGGCGTCACAAGACCCCGCT |
| | | GATCTACCGCGCTACCGCGCAGTGGTTTATCGGCATGGACAAAGAGC |
| | | CGACCAGCGGCGACACCCTGCGTGTGCGCTCGCTCAAAGCGATCGAA |
| | | GAGACCAAGTTTGTCCCGGCCTGGGGCCAGGCGCGCCTGCACTCGAT |
| | | GATCGCCAACCGCCCGGACTGGTGCATCTCCCGCCAGCGCAACTGGG |
| | | GCGTGCCGATTCCGTTCTTCCTGAACAAGGAAAGCGGCGAGCTGCAC |
| | | CCACGTACCGTTGAACTGATGGAAGCAGTGGCGCTGCGCGTTGAGCA |
| | | GGAAGGCATCGAAGCCTGGTTCAAGCTGGACGCCGCCGAACTGCTGG |
| | | GCGACGAAGCGCCGCTGTACGACAAGATCAGCGACACCCTCGACGTG |
| | | TGGTTCGACTCGGGTACCACCCACTGGCACGTGCTGCGCGGTTCGCAC |
| | | CCGATGGGTCACGCCACCGGCCCGCGTGCCGACCTGTACCTGGAAGG |
| | | CTCGGACCAACACCGTGGCTGGTTCCACTCGTCGTTGCTGACCGGCTG |
| | | CGCCATCGACAACCACGCGCCGTACCGCGAACTGCTGACCCACGGCT |
| | | TCACCGTCGACGAGACGGGCCGCAAGATGTCCAAGTCGCTGAAAAAC |
| | | GTGATCGAGCCGAAAAAGATCAACGACACCCTGGGCGCCGATATCAT |
| | | GCGTCTGTGGGTCGCCTCGACCGATTACTCGGGCGAAATCGCCGTGTC |
| | | GGACCAGATCCTGGCCCGTAGCGCCGATGCCTACCGCCGTATCCGTA |
| | | ATACCGCACGCTTCCTGCTGTCGAACCTGACCGGTTTCAACCCGGCCA |
| | | CCGACATCCTGCCGGCCGAGGACATGCTCGCCCTGGACCGTTGGGCC |
| | | GTGGACCGTACGCTGTTGCTGCAGCGCGAGTTGCAGGAACACTACGG |
| | | CGAATACCGTTTCTGGAACGTGTACTCCAAGATCCACAACTTCTGCGT |
| | | GCAGGAGCTGGGTGGTTTCTACCTCGATATCATCAAGGACCGCCAGT |
| | | ACACCACCGGCGCCAACAGCAAGGCGCGCCGCTCGGCGCAGACCGC |
| | | GCTGTACCACATCTCTGAAGCGCTGGTGCGCTGGATCGCACCGATCCT |
| | | GGCCTTCACCGCTGACGAACTGTGGGAATACCTGCCGGGCGAGCGTA |
| | | ACGAATCGGTGATGCTCAACACCTGGTACGAAGGCCTGACCGAATTG |
| | | CCGGCCAACTTCGAACTGGGCCGCGAGTACTGGGAAGGCGTGATGGC |
| | | CGTCAAGGTTGCGGTGAACAAGGAGCTGGAAGTTCAGCGCGCGGCCA |
| | | AGGCCGTCGGTGGCAACCTGCAAGCCGAAGTCACCCTGTTTGCCGAG |
| | | GAAGGCCTGACCGCCGACCTGGCCAAGCTGAGCAACGAACTGCGCTT |
| | | CGTACTGATCACCTCGACCGCGAGCCTGGCACCGTTTGCCCAGGCACC |
| | | TGCGGACGCAGTGGCCACCGAAGTGCCGGGCCTCAAGCTCAAAGTGG |
| | | TCAAGTCGGCCTTTCCTAAGTGCGCCCGTTGCTGGCACTGCCGTGAAG |
| | | ACGTCGGCGTGAACCCAGAGCATCCGGAAATCTGCGGTCGTTGCGTC |
| | | GACAACATCAGCGGTGCTGGCGAGGTTCGCCACTATGCCTAA |
| 112 | DP68 NADH- | ATGACTACAGGCAGTGCTCTGTACATCCCGCCTTACAAGGCAGACGA |
| | quinone | CCAGGATGTGGTTGTCGAACTCAATAACCGTTTTGGCCCTGACGCCTT |
| | oxidoreductase | CACCGCCCAGGCCACACGCACCGGTATGCCGGTGCTGTGGGTGGCGC |
| | subunit C/D | GCGCCAAGCTCGTCGAAGTCCTGAGCTTCCTGCGCAACCTGCCCAAG |
| | | CCGTACGTCATGCTTTATGACCTGCATGGCGTGGACGAGCGTCTGCGC |
| | | ACCAAGCGTCAAGGTTTGCCGAGCGGTGCCGATTTCACCGTGTTCTAC |
| | | CACTTGATGTCGCTGGAACGTAACAGCGACGTGATGATCAAGGTCGC |
| | | GCTGTCCGAAAGCGACTTGAGCATCCCGACCGTCACCGGTATCTGGC |
| | | CGAATGCCAGCTGGTACGAGCGCGAAGTTTGGGACATGTTCGGTATC |
| | | GACTTCCCGGGCCACCCGCACCTGACGCGCATCATGATGCCGCCGAC |
| | | CTGGGAAGGTCACCCGCTGCGCAAGGACTTTCCTGCCCGCGCAACCG |
| | | AATTCGACCCGTTCAGCCTCAACCTCGCCAAGCAGCAGCTTGAAGAA |
| | | GAAGCTGCACGCTTCCGTCCGGAAGACTGGGGCATGAAACGCTCCGG |
| | | CACCAACGAGGACTACATGTTCCTCAACCTGGGCCCGAACCACCCTTC |
| | | GGCTCACGGTGCCTTCCGTATCATCCTGCAACTGGACGGCGAAGAAA |
| | | TCGTCGACTGTGTGCCGGACATCGGTTACCACCACCGTGGTGCCGAG |
| | | AAGATGGCCGAGCGCCAGTCCTGGCACAGCTTCATCCCGTACACCGA |
| | | CCGTATCGACTACCTCGGCGGCGTGATGAACAACCTGCCGTACGTGCT |
| | | GTCGGTCGAGAAGCTGGCCGGTATCAAGGTGCCGGACCGCGTCGACA |
| | | CCATCCGCATCATGATGGCCGAGTTCTTCCGCATCACCAGCCACCTGC |
| | | TGTTCCTGGGTACCTATATCCAGGACGTTGGCGCCATGACCCCGGTGT |
| | | TCTTCACCTTCACCGACCGTCAACGCGCCTACAAGGTGATCGAAGCCA |
| | | TCACCGGTTTCCGCCTGCACCCGGCCTGGTATCGCATCGGCGGCGTGG |
| | | CGCACGACCTGCCGAACGGCTGGGAGCGCCTGGTCAAGGAATTCATC |
| | | GACTGGATGCCCAAGCGTCTGGACGAGTACCAAAAGGCTGCGCTGGA |
| | | CAACAGCATCCTCAAGGGTCGTACCATCGGCGTCGCGCAGTACAACA |
| | | CCAAAGAAGCCCTGGAATGGGGCGTCACTGGTGCCGGCCTGCGTTCG |
| | | ACCGGCTGCGACTTCGACCTGCGTAAAGCACGGCCGTACTCGGGCTA |
| | | CGAGAACTTCGAGTTCGAAGTGCCGCTGGCCGCCAATGGCGATGCCT |
| | | ACGACCGGTGCATCGTGCGCGTTGAAGAAATGCGCCAGAGCCTGAAG |
| | | ATCATCGAGCAGTGCATGCGCAACATGCCGGCTGGCCCGTACAAGGC |
| | | GGATCATCCGCTGACCACACCGCCGCCGAAAGAGCGCACGCTGCAGC |
| | | ACATCGAAACCCTGATCACGCACTTCCTGCAAGTTTCGTGGGGCCCGG |
| | | TGATGCCGGCCAACGAATCCTTCCAGATGATCGAAGCGACCAAGGGT |
| | | ATCAACAGTTATTACCTGACGAGCGATGGCGGCACCATGAGCTACCG |
| | | CACCCGGATTCGTACCCCAAGCTTTGCCCACTTGCAGCAGATCCCTTC |
| | | GGTGATCAAAGGCGAGATGGTCGCGGACTTGATTGCGTACCTGGGTA |
| | | GTATCGATTTCGTTATGGCCGACGTGGACCGCTAA |
| 113 | DP68 Protein | ATGGACGACAACAAGAAGAAAGCCTTGGCTGCGGCCCTGGGTCAGAT |
| | RecA | CGAACGTCAATTCGGCAAGGGTGCCGTAATGCGTATGGGCGATCACG |
| | | ACCGTCAGGCGATCCCGGCTATTTCCACTGGCTCTCTGGGTCTGGACA |
| | | TCGCACTCGGCATTGGCGGCCTGCCAAAAGGCCGTATCGTTGAAATCT |
| | | ACGGTCCTGAATCTTCCGGTAAAACCACCCTGACCCTGTCGGTGATTG |
| | | CCCAGGCGCAAAAAATGGGCGCCACCTGTGCGTTCGTCGACGCCGAG |
| | | CACGCCCTGGACCCGGAATACGCCGGTAAGCTGGGCGTCAACGTTGA |
| | | CGACCTGCTGGTTTCCCAGCCGGACACCGGTGAGCAAGCCCTGGAAA |
| | | TCACCGACATGCTGGTGCGCTCCAACGCCATCGACGTGATCGTGGTCG |
| | | ACTCCGTGGCTGCCCTGGTACCGAAAGCTGAAATCGAAGGCGAAATG |
| | | GGCGACATGCACGTGGGCCTGCAAGCCCGCCTGATGTCCCAGGCGCT |
| | | GCGTAAAATTACCGGTAACATCAAGAACGCCAACTGCCTGGTGATCT |
| | | TCATCAACCAGATCCGTATGAAGATCGGCGTAATGTTCGGCAGCCCG |
| | | GAAACCACTACCGGTGGTAACGCGCTGAAGTTCTACGCTTCGGTCCGT |
| | | CTGGACATCCGCCGTACCGGCGCGGTGAAGGAAGGTGACGAAGTTGT |
| | | TGGTAGCGAAACTCGCGTTAAAGTCGTGAAGAACAAGGTCGCTCCGC |
| | | CTTTCCGTCAGGCAGAGTTCCAGATTCTCTACGGCAAGGGTATCTACC |
| | | TGAACGGCGAGATGATTGACCTGGGCGTACTGCACGGTTTCGTCGAG |
| | | AAGTCCGGTGCCTGGTATGCCTACAACGGCAGCAAGATCGGTCAGGG |
| | | CAAGGCCAACTCGGCCAAGTTCCTGGCAGACAACCCGGATATCGCTG |
| | | CCACGCTTGAGAAGCAGATTCGCGACAAGCTGCTGACCCCAGCGCCA |
| | | GACGTGAAAGCTGCCGCCAACCGCGAGCCGGTTGAAGAAGTGGAAG |
| | | AAGCTGACACTGATATCTGA |
| 114 | DP68 RNA | ATGTCCGGAAAAGCGCAACAACAGTCTCGTATTAAAGAGTTGATCAC |
| | polymerase sigma | CCTTGGTCGTGAGCAGAAATATCTGACTTACGCAGAGGTCAACGATC |
| | factor RpoD | ACCTGCCTGAGGATATTTCAGATCCTGAGCAGGTGGAAGACATCATC |
| | | CGCATGATTAATGACATGGGGATCCCCGTACACGAGAGTGCTCCGGA |
| | | TGCGGACGCCCTTATGTTGGCCGACTCCGATACCGACGAGGCAGCTG |
| | | CTGAAGAAGCGGCTGCTGCGCTGGCAGCGGTGGAGACCGACATCGGT |
| | | CGTACGACTGACCCTGTGCGCATGTATATGCGTGAAATGGGTACCGTC |
| | | GAGCTGCTGACACGTGAAGGCGAAATCGAAATCGCCAAACGTATTGA |
| | | AGAGGGTATCCGTGAAGTGATGGGCGCAATCGCGCACTTCCCTGGCA |
| | | CGGTTGACCACATTCTCTCCGAGTACACTCGCGTCACCACCGAAGGTG |
| | | GCCGCCTGTCTGACGTTCTGAGCGGCTACATCGACCCGGACGACGGC |
| | | ATTGCGCCGCCTGCCGCCGAAGTACCGCCGCCCGTCGATGCGAAAGC |
| | | CGCGAAGGCTGACGACGACACCGAAGACGACGATGCTGAAGCCAGC |
| | | AGCGACGACGAAGATGAAGTTGAAAGCGGCCCGGACCCGATCATCGC |
| | | AGCCCAGCGTTTCGGTGCGGTTTCCGATCAAATGGAAATCACCCGCA |
| | | AGGCCCTGAAAAAGCACGGTCGCTCCAACAAGCTGGCGATTGCCGAG |
| | | CTGGTGGCCCTGGCTGAGCTGTTCATGCCGATCAAGCTGGTACCGAA |
| | | GCAATTCGAAGGCTTGGTTGAGCGTGTTCGCAGTGCCCTTGAACGTCT |
| | | GCGTGCGCAAGAACGCGCAATCATGCAGCTGTGTGTACGTGATGCAC |
| | | GTATGCCGCGGGCTGACTTCCTGCGCCAGTTCCCGGGCAACGAAGTA |
| | | GACGAAAGCTGGACCGACGCACTGGCCAAAGGCAAGGCGAAATACG |
| | | CCGAAGCCATTGGTCGCCTGCAGCCGGACATCATCCGTTGCCAGCAG |
| | | AAGCTGACCGCGCTTGAGACCGAAACCGGTCTGACGATTGCTGAAAT |
| | | CAAAGACATCAACCGTCGCATGTCGATCGGTGAGGCCAAGGCCCGCC |
| | | GCGCGAAGAAAGAGATGGTTGAAGCGAACTTGCGTCTGGTGATCTCG |
| | | ATCGCCAAGAAGTACACCAACCGTGGTCTGCAATTCCTCGATCTGATC |
| | | CAGGAAGGCAACATCGGCTTGATGAAGGCGGTGGACAAGTTCGAATA |
| | | CCGTCGCGGCTACAAGTTCTCGACTTATGCCACCTGGTGGATCCGTCA |
| | | GGCGATCACTCGCTCGATCGCCGACCAGGCTCGCACCATCCGTATTCC |
| | | GGTGCACATGATCGAGACGATCAACAAGCTCAACCGTATTTCCCGGC |
| | | AGATGTTGCAGGAAATGGGTCGCGAACCGACCCCGGAAGAGCTGGGC |
| | | GAACGCATGGAAATGCCTGAGGATAAAATCCGCAAGGTATTGAAGAT |
| | | CGCTAAAGAGCCGATCTCCATGGAAACGCCGATTGGTGATGACGAAG |
| | | ACTCCCACCTGGGTGACTTCATCGAAGACTCGACCATGCAGTCGCCA |
| | | ATCGATGTCGCCACTGTTGAGAGCCTTAAAGAAGCGACTCGCGACGT |
| | | ACTGTCCGGCCTCACTGCCCGTGAAGCCAAGGTACTGCGCATGCGTTT |
| | | CGGCATCGACATGAATACCGACCACACCCTTGAGGAAGTCGGTAAGC |
| | | AGTTTGACGTGACCCGCGAGCGGATCCGTCAGATCGAAGCCAAGGCG |
| | | CTGCGCAAGTTGCGCCACCCGACGCGAAGCGAGCATCTGCGCTCCTT |
| | | CCTCGACGAGTGA |
| 115 | DP68 DNA- | ATGGCTTACTCATATACTGAGAAAAAACGTATCCGCAAGGACTTTAG |
| | directed RNA | CAAGTTGCCGGACGTCATGGATGTCCCGTACCTTCTGGCTATCCAGCT |
| | polymerase | GGATTCGTATCGTGAATTCTTGCAGGCGGGAGCGACCAAAGATCAGT |
| | subunit beta | TCCGCGACGTGGGCCTGCATGCGGCCTTCAAATCCGTTTTCCCGATCA |
| | | TCAGCTACTCCGGCAATGCTGCGCTGGAGTACGTGGGTTATCGCCTGG |
| | | GCGAACCGGCATTTGATGTCAAAGAATGCGTGTTGCGCGGTGTTACG |
| | | TACGCCGTACCTTTGCGGGTAAAAGTCCGCCTGATCATTTTCGACAAA |
| | | GAATCGTCGAACAAAGCGATCAAGGACATCAAAGAGCAAGAAGTCT |
| | | ACATGGGCGAAATCCCACTGATGACTGAAAACGGTACCTTCGTAATC |
| | | AACGGTACCGAGCGTGTTATTGTTTCCCAGCTGCACCGTTCCCCGGGC |
| | | GTGTTCTTCGACCACGACCGCGGCAAGACGCACAGCTCCGGTAAACT |
| | | CCTGTACTCCGCGCGGATCATTCCGTACCGCGGTTCGTGGTTGGACTT |
| | | CGAGTTCGACCCGAAAGACTGCGTGTTCGTGCGTATCGACCGTCGTCG |
| | | CAAGCTGCCGGCCTCGGTACTGCTGCGCGCGCTCGGTTACACCACTGA |
| | | GCAGGTGCTGGACGCTTTCTACACCACCAACGTATTCAGCCTGAAGG |
| | | ATGAAACCCTCAGCCTGGAGCTGATTGCTTCGCGTCTGCGTGGTGAAA |
| | | TTGCCGTTCTGGACATTCAGGACGAAAACGGCAAAGTGATCGTTGAA |
| | | GCGGGTCGTCGTATTACTGCGCGCCACATCAACCAGATCGAAAAAGC |
| | | CGGCATCAAGTCGCTGGAAGTGCCTCTGGACTACGTCCTGGGTCGCA |
| | | CCACCGCCAAGGTTATCGTTCACCCGGCTACAGGCGAAATCCTGGCT |
| | | GAGTGCAACACCGAGCTGAACACCGAAATCCTGGCAAAAATCGCCAA |
| | | GGCCCAGGTTGTTCGCATCGAGACCCTGTACACCAACGACATCGACT |
| | | GCGGTCCGTTCATCTCCGACACACTGAAGATCGACTCCACCAGCAAC |
| | | CAATTGGAAGCGCTGGTCGAGATCTATCGCATGATGCGTCCTGGTGA |
| | | GCCACCGACCAAAGACGCTGCCGAGACCCTGTTCAACAACCTGTTCTT |
| | | CAGCCCTGAGCGTTATGACCTGTCTGCGGTCGGCCGGATGAAGTTCA |
| | | ACCGTCGTATCGGTCGTACCGAGATCGAAGGTTCGGGCGTGCTGTGC |
| | | AAGGAAGATATCGTCGCGGTACTGAAGACTCTGGTCGACATCCGTAA |
| | | CGGTAAAGGCATCGTCGATGACATCGACCACCTGGGTAACCGTCGTG |
| | | TTCGCTGCGTAGGCGAAATGGCCGAAAACCAGTTCCGCGTTGGCCTT |
| | | GTGCGTGTTGAACGTGCGGTCAAAGAGCGTCTGTCGATGGCTGAAAG |
| | | CGAAGGCCTGATGCCGCAAGACCTGATCAACGCCAAGCCAGTGGCTG |
| | | CGGCAGTGAAAGAGTTCTTCGGTTCCAGCCAGCTTTCCCAGTTCATGG |
| | | ACCAGAACAACCCGCTCTCCGAGATCACCCACAAGCGCCGTGTTTCT |
| | | GCACTGGGCCCGGGCGGTCTGACCCGTGAGCGTGCTGGCTTTGAAGT |
| | | TCGTGACGTACACCCGACGCACTACGGTCGTGTTTGCCCGATCGAAAC |
| | | GCCGGAAGGTCCGAACATCGGTCTGATCAACTCCCTGGCCGCTTATGC |
| | | GCGCACCAACCAGTACGGCTTCCTCGAGAGCCCGTACCGCGTGGTGA |
| | | AAGACGCTCTGGTCACCGACGAGATCGTATTCCTGTCCGCCATCGAA |
| | | GAAGCTGATCACGTGATCGCTCAGGCTTCGGCCACGATGAACGACAA |
| | | GAAAGTCCTGATCGACGAGCTGGTAGCTGTTCGTCACTTGAACGAGTT |
| | | CACCGTCAAGGCGCCGGAAGACGTCACCTTGATGGACGTTTCGCCGA |
| | | AGCAGGTAGTTTCGGTTGCAGCGTCGCTGATCCCGTTCCTGGAACACG |
| | | ATGACGCCAACCGTGCGTTGATGGGTTCCAACATGCAGCGTCAAGCT |
| | | GTACCAACCCTGCGCGCTGACAAGCCGCTGGTAGGTACCGGCATGGA |
| | | GCGTAACGTAGCCCGTGACTCCGGCGTTTGCGTCGTAGCCCGTCGTGG |
| | | CGGCGTGATCGACTCCGTTGATGCCAGCCGTATCGTGGTTCGTGTTGC |
| | | CGATGATGAAGTTGAAACTGGCGAAGCCGGTGTCGACATCTACAACC |
| | | TGACCAAATACACCCGCTCGAACCAGAACACCTGCATCAACCAGCGT |
| | | CCGCTGGTGAGCAAGGGTGACCGCGTTCAGCGTAGCGACATCATGGC |
| | | CGACGGCCCGTCCACTGACATGGGTGAACTGGCTCTGGGTCAGAACA |
| | | TGCGCATCGCGTTCATGGCATGGAACGGCTTCAACTTCGAAGACTCCA |
| | | TCTGCCTGTCCGAGCGTGTTGTTCAAGAAGACCGTTTCACCACGATCC |
| | | ACATTCAGGAACTGACCTGTGTGGCACGTGATACCAAGCTTGGGCCA |
| | | GAGGAAATCACTGCAGACATCCCGAACGTGGGTGAAGCTGCACTGAA |
| | | CAAGCTGGACGAAGCCGGTATCGTTTACGTAGGTGCTGAAGTTGGCG |
| | | CAGGCGACATCCTGGTAGGTAAGGTCACTCCGAAAGGCGAGACCCAA |
| | | CTGACTCCGGAAGAGAAGCTGCTGCGTGCCATCTTCGGTGAAAAAGC |
| | | CAGCGACGTTAAAGACACCTCCCTGCGTGTACCTACCGGTACCAAGG |
| | | GTACTGTTATCGACGTACAGGTCTTCACCCGTGACGGCGTTGAGCGTG |
| | | ATGCTCGTGCACTGTCCATCGAGAAGACTCAACTCGACGAGATCCGC |
| | | AAGGACCTGAACGAAGAGTTCCGTATCGTTGAAGGCGCGACCTTCGA |
| | | ACGTCTGCGTTCCGCTCTGGTAGGCCACAAGGCTGAAGGCGGCGCAG |
| | | GTCTGAAGAAAGGTCAGGACATCACCGACGAAGTACTCGACGGTCTT |
| | | GAGCACGGCCAGTGGTTCAAACTGCGCATGGCTGAAGATGCTCTGAA |
| | | CGAGCAGCTCGAGAAGGCCCAGGCCTACATCGTTGATCGCCGTCGTC |
| | | TGCTGGACGACAAGTTCGAAGACAAGAAGCGCAAACTGCAGCAGGG |
| | | CGATGACCTGGCTCCAGGCGTGCTGAAAATCGTCAAGGTTTACCTGG |
| | | CAATCCGTCGCCGCATCCAGCCGGGCGACAAGATGGCCGGTCGTCAC |
| | | GGTAACAAAGGTGTGGTCTCCGTGATCATGCCGGTTGAAGACATGCC |
| | | GCACGATGCCAATGGCACCCCGGTCGACGTCGTCCTCAACCCGTTGG |
| | | GCGTACCTTCGCGTATGAACGTTGGTCAGATCCTCGAAACCCACCTGG |
| | | GCCTCGCGGCCAAAGGTCTGGGCGAGAAGATCAACCGTATGATCGAA |
| | | GAGCAGCGCAAGGTTGCTGACCTGCGTAAGTTCCTGCACGAGATCTA |
| | | CAACGAGATCGGCGGTCGCAACGAAGAGCTGGACACCTTCTCCGACC |
| | | AGGAAATCCTGGACTTGGCGAAGAACCTGCGCGGCGGCGTTCCAATG |
| | | GCTACCCCGGTGTTCGACGGTGCCAAGGAAAGCGAAATCAAGGCCAT |
| | | GCTGAAACTGGCAGACCTGCCGGAAAGCGGCCAGATGCAGCTGTTCG |
| | | ACGGCCGTACCGGCAACAAGTTTGAGCGCCCGGTTACTGTTGGCTAC |
| | | ATGTACATGCTGAAGCTGAACCACTTGGTAGACGACAAGATGCACGC |
| | | TCGTTCTACCGGTTCGTACAGCCTGGTTACCCAGCAGCCGCTGGGTGG |
| | | TAAGGCTCAGTTCGGTGGTCAGCGTTTCGGGGAGATGGAGGTCTGGG |
| | | CACTGGAAGCATACGGTGCTGCATACACTCTGCAAGAAATGCTCACA |
| | | GTGAAGTCGGACGATGTGAACGGTCGGACCAAGATGTACAAAAACAT |
| | | CGTGGACGGCGATCACCGTATGGAGCCGGGCATGCCCGAGTCCTTCA |
| | | ACGTGTTGATCAAAGAAATTCGTTCCCTCGGCATCGATATCGATCTGG |
| | | AAACCGAATAA |
| 116 | DP69 Glutamine- | GTGCGCGAGGACCTGGCCAGCGGAAAGCACCAGGCGATCAAGACCC |
| | IRNA ligase | GCTTCCCGCCGGAGCCGAACGGCTACCTGCACATCGGCCACGCCAAG |
| | | TCGATCTGCCTGAACTTCGGCATCGCCGGTGAGTTCAGCGGCGTCTGC |
| | | AACCTGCGTTTCGACGACACCAATCCGGCCAAGGAAGACCCGGAGTA |
| | | CGTGGCCGCGATCCAGGACGACGTGCGCTGGCTGGGCTTTGAATGGA |
| | | ACGAGCTGCGCCACGCCTCGGACTACTTCCAGACCTATTACCTGGCCG |
| | | CCGAGAAGCTGATCGAACAGGGCAAGGCCTACGTCTGCGACCTGTCG |
| | | GCCGAGGAAGTGCGCGCCTACCGCGGCACCCTGACCGAGCCGGGCCG |
| | | CCCGTCGCCGTGGCGTGACCGCAGCGTCGAGGAGAACCTCGACCTGT |
| | | TCCGCCGCATGCGTGCCGGTGAATTCCCCGATGGCGCGCGCACCGTG |
| | | CGCGCCAAGATCGACATGGCCAGCGGCAACATCAACCTGCGTGATCC |
| | | GGCGCTGTACCGCATCAAGCACGTCGAGCACCAGAACACCGGCAACG |
| | | CGTGGCCGATCTACCCGATGTACGACTTCGCCCATGCGCTGGGCGATT |
| | | CGATCGAGGGCATCACCCACTCGCTGTGCACGCTGGAATTCGAAGAC |
| | | CACCGCCCGCTGTACGACTGGTGCGTGGACAACGTCGACTTCGCCCA |
| | | CGATGACGCGCTGACCCAGCCGCTGGTCGACGCCGGCCTGCCGCGCG |
| | | AAGCGGCCAAACCGCGCCAGATCGAGTTCTCGCGCCTGAACATCAAC |
| | | TACACGGTGATGAGCAAGCGCAAGCTGATGGCGCTGGTCACCGAACA |
| | | GCTGGTGGACGGCTGGGAAGACCCGCGCATGCCGACCCTGCAGGGCC |
| | | TGCGTCGCCGTGGCTACACCCCGGCAGCGATGCGCCTGTTCGCCGAG |
| | | CGCGTGGGCATCAGCAAGCAGAATTCGCTGATCGATTTCAGCGTGCT |
| | | GGAAGGCGCGCTGCGCGAAGACCTGGACAGCGCCGCACCGCGCCGC |
| | | ATGGCCGTGGTCGACCCGGTCAAGCTGGTGCTGACCAACCTGGCCGA |
| | | AGGCCACGAAGAGCAGCTGACCTTCAGCAACCACCCGAAGGACGAG |
| | | AGCTTCGGTACCCGCGAAGTGCCGTTCGCACGTGAAGTGTGGATCGA |
| | | CCGCGAGGACTTCGCCGAAGTGCCGCCGAAGGGCTGGAAGCGCCTGG |
| | | TTCCCGGTGGTGAAGTGCGCCTGCGCGGCGCCGGCATCATCCGCTGC |
| | | GACGACGTGATCAAGGATGCCGACGGCACCATCACCGAGCTGCGCGG |
| | | CTGGCTGGATCCGGAATCGCGCCCGGGCATGGAAGGCGCCAACCGCA |
| | | AGGTCAAGGGCACCATCCACTGGGTCAGCGCGGTGCACGGTGTGCCG |
| | | GCCGAGATCCGCCTGTATGACCGCCTGTTCTCGGTGCCGAACCCGGAC |
| | | GATGAATCGGAAGGCAAGACCTACCGCGACTACCTCAATCCGGACTC |
| | | GCGCCGCACCGTCACCGGCTATGTCGAGCCGGCGGCTGCCAGCGCTG |
| | | CGCCGGAACAGTCGTTCCAGTTCGAGCGCACCGGCTACTTCGTTGCCG |
| | | ACCGCCGCGACCACACCGAAGCCAAGCCGGTGTTCAACCGCAGCGTG |
| | | ACCCTGCGCGACACCTGGTCGGCCTGA |
| 117 | DP69 DNA | ATGACCGACGAACAGAACACCCCGGCAAACAACGGCAACTACGACG |
| | gyrase subunit B | CCAACAGCATTACGGCCCTGGAAGGCCTGGAGGCTGTCCGCAAGCGC |
| | | CCAGGCATGTACATCGGCGACGTCCATGACGGCACCGGCCTGCATCA |
| | | CATGGTGTTCGAGGTCGTCGACAACTCAATCGACGAAGCCCTCGCCG |
| | | GCCATGCCGACCACGTCTCGGTGACGATCCATGCCGATGGCTCGGTA |
| | | GGCGTGTCCGACAACGGTCGCGGCATCCCGACGGGCAAGCACGAGCA |
| | | GATGAGCAAGAAGCTCGACCGCGATGTGTCTGCAGCCGAAGTGGTGA |
| | | TGACGGTCCTGCACGCAGGCGGCAAGTTCGACGACAACAGCTACAAG |
| | | GTTTCCGGCGGCCTGCACGGCGTGGGCGTCAGCGTGGTCAACGCGCT |
| | | GTCGCAGAAGCTGGTCCTGGATATCTACCAGGGTGGCTTCCACTACCA |
| | | GCAGGAGTACGCCGACGGCGCAGCACTGCATCCGCTGAAGCAGATCG |
| | | GCCCCAGCACCAAGCGCGGGACCACCCTGCGCTTCTGGCCCTCGGTA |
| | | AAGGCTTTCCACGACAACGTGGAATTCCACTACGACATCCTGGCCCG |
| | | GCGCCTGCGCGAACTGTCCTTCCTCAATTCCGGCGTCAAGATCGTGCT |
| | | GGTGGACGAGCGTGGTGATGGCCGCCGCGACGACTTCCATTACGAGG |
| | | GCGGCATCCGCAGCTTCGTGGAGCATCTGGCGCAGTTGAAGACGCCG |
| | | TTGCACCCGAACGTGATCTCGGTGACCGGCGAATCCAATGGCATCAC |
| | | CGTGGAAGTGGCGCTGCAGTGGACCGACTCCTACCAGGAGACGATGT |
| | | ACTGCTTCACCAACAACATTCCGCAGAAGGACGGCGGTACCCACCTG |
| | | GCCGGCTTCCGTGGCGCATTGACCCGCGTGCTCAACAACTACATCGA |
| | | GCAGAACGGCATCGCCAAGCAGGCCAAGATCAACCTGACCGGCGATG |
| | | ACATGCGCGAAGGCATGATCGCGGTGCTGTCGGTGAAGGTGCCGGAT |
| | | CCCAGCTTCTCCAGCCAGACCAAGGAAAAGCTGGTCAGCTCGGATGT |
| | | GCGCCCGGCCGTGGAAAGCGCGTTCGGCCAGCGCCTGGAAGAGTTCC |
| | | TGCAGGAAAACCCGAACGAAGCCAAGGCCATCGCCGGCAAGATCGTC |
| | | GACGCTGCCCGTGCCCGCGAAGCGGCGCGCAAGGCCCGCGACCTGAC |
| | | CCGCCGCAAGGGTGCGCTGGATATCGCCGGCCTGCCGGGCAAGCTGG |
| | | CCGACTGCCAGGAAAAGGATCCGGCGCTGTCCGAACTGTTCATCGTC |
| | | GAGGGTGACTCGGCAGGTGGTTCGGCCAAGCAGGGTCGCAACCGCAA |
| | | GAACCAGGCGGTGCTGCCGCTGCGCGGCAAGATCCTCAACGTGGAAC |
| | | GTGCGCGCTTCGACCGCATGCTGGCGTCCGACCAGGTGGGTACGCTG |
| | | ATCACCGCGCTGGGTACCGGCATCGGTCGTGACGAGTACAACCCGGA |
| | | CAAGCTGCGGTACCACAAGATCATCATCATGACCGACGCCGACGTCG |
| | | ACGGCGCGCACATCCGCACCCTGCTGCTGACGTTCTTCTACCGTCAGA |
| | | TGCCGGAGCTGATCGAGCGCGGTTATGTCTATATCGGCCTGCCGCCGT |
| | | TGTACAAGATCAAGCAGGGCAAGCAGGAGCTGTACCTGAAGGACGA |
| | | CCCGGCGCTGGACAGCTATCTGGCCAGCAGCGCGGTGGAGAACGCTG |
| | | GGCTGGTGCCGGCCAGCGGCGAGCCGCCGATCGACGGCGTGGCACTG |
| | | GAAAAGCTGCTGCTCGCCTACGCTGCCGCGCAGGACACGATCAACCG |
| | | CAATACCCACCGCTACGACCGCAACCTGCTCGAAGCGCTGGTCGACT |
| | | TCATGCCGCTGGAGCTGGAAAACCTGCGCACTGCAGGTCCTGGCGAA |
| | | GGTCTGGACGCGTTGGCCAAGCACCTCAACCAGGGCAACCTCGGCAG |
| | | CGCCCGCTTCACCCTGGAACTGCAGGAACCCAACGAGCAGCGTCCGG |
| | | CGGCCGTACTGGTGACCCGCAGCCACATGGGCGAACAGCACATCCAG |
| | | GTGCTGCCGCTGTCCGCGCTGGAAAGCGGCGAACTGCGCGGCATCCA |
| | | TCAGGCAGCGCAGCTGCTGCACGGTCTGGTCCGCGAAGGCGCGGTCA |
| | | TCACCCGTGGCGCCAAGTCGATCGAGATCGACTCGTTCGCACAGGCC |
| | | CGCAACTGGCTGTTGGACGAAGCCAAGCGCGGCCGGCAGATCCAGCG |
| | | ATTCAAGGGTCTGGGCGAAATGAATCCGGAACAGCTGTGGGATACCA |
| | | CCGTCAATCCCGATACCCGTCGCCTGCTGCAGGTGCGCATCGAAGAC |
| | | GCGGTGGCCGCTGACCAGATCTTCAGCACCCTGATGGGTGATGTGGT |
| | | CGAACCGCGTCGTGACTTCATCGAAGACAACGCGTTGAAGGTCGCCA |
| | | ACCTGGATATCTGA |
| 118 | DP69 Isoleucine | GTGAGCCAGGACTACAAGACCACCCTCAACCTGCCGGCCACCGAATT |
| | tRNA ligase | CCCGATGCGCGGCGACCTGCCCAAGCGCGAGCCGGGCATTCTGGCGC |
| | | GCTGGGAAGAGCAGGGGCTCTACCAGCAGCTGCGCGACAACGCCGCC |
| | | GGCCGCCCGCTGTTCGTGCTGCATGACGGCCCGCCGTACGCCAATGC |
| | | GCGCATCCACCTGGGCCATGCGGTCAACAAGATCCTCAAGGACATCA |
| | | TCGTCAAGTCGCGCTACCTGGCCGGCTTCGATGCGCCCTACGTGCCGG |
| | | GCTGGGACTGCCATGGCCTGCCGATCGAAATCGCGGTGGAAAAGAAG |
| | | TGGGGCAAGGTCGGGGTGAAGCTCGATGCGGTCGAGTTCCGGCAGAA |
| | | GTGCCGCGAGTTCGCCGAAGAACAGATCGACATCCAGCGTGCCGACT |
| | | TCAAGCGCCTGGGCGTCACCGGCGACTGGGACAACCCGTACAAGACC |
| | | CTAAGCTTCGATTTCGAGGCCAACGAGATCCGTGCGCTGTCCAAGATC |
| | | GTGGCCAACGGCCATCTGCTGCGTGGCGCCAAGCCGGTCTACTGGTG |
| | | CTTCGACTGCGGCTCGGCACTGGCCGAGGCCGAGATCGAGTACCACG |
| | | AGAAGACCTCGCCGGCGATCGACGTGGCCTACACCGCGCGTGATCCG |
| | | CAGGCGGTGGCGCAGGCGTTCGGCGTCAGCCTGCCGGCCGATGTCGA |
| | | AGTGGCGGTGCCGATCTGGACCACCACTCCGTGGACGCTGCCGGCTT |
| | | CGCTGGCGGTGTCGCTGGGCGCGGACATCCGCTACGTGCTGGCCGAA |
| | | GGCCCGGCGCACAACGGCAAGCGCCGTTGGCTGGTGCTGGCTGCTGC |
| | | GCTGGCCGAACGGTCGCTGCAGCGCTACGGCGTGGACGCGGTGGTGC |
| | | TGCACGGTGAAGCCGAAGGTTCGGCGCTGGAAAACCAGCTGCTGGCG |
| | | CACCCGTTCTACCCGGAGCGCGAGATCCCCGTGCTCAACGGCGAACA |
| | | CGTGTCCGACGAGGACGGTACCGGTGCGGTGCACACTGCCCCCGGCC |
| | | ACGGCCAGGAAGACTACGTGGTCAGCCAGAAGTACGGCCTGCTGGAG |
| | | AAGTACAACGCCGGCCAGATCAATCCGGTCGACGGTGCGGGCGTGTA |
| | | CCTGGCGTCCACCCCGCCCGCCGGTGACCTGGTGCTGGCCGGTACCC |
| | | ACATCTGGAAGGCGCAGCAGCCGATCATCGAAGTGCTGGCCGCCAGC |
| | | GGCGCGCTGCTCAAGGCCGTGGAGATCGTGCACAGTTATCCGCATTG |
| | | TTGGCGCCACAAGAAGACCCCGCTGGTGTTCCGCGCCACCCCGCAGT |
| | | GGTTCATTTCGATGGACAAGGCCAACCTGCGCAACGATGCGCTGGCC |
| | | GCGATCGATACCGTCGGCTGGTTCCCGAGCTGGGGCAAGGCGCGCAT |
| | | CCAAAGCATGATCGACGGCCGCCCGGACTGGACCATCTCGCGCCAGC |
| | | GCACCTGGGGCGTGCCGATCGCGCTGTTCACCCACCGCCAGACCGGC |
| | | GAGATCCACCCGCGTTCGGTGGAGCTGATGCAGCAGGTGGCCGACCG |
| | | CGTTGAAGCCGAAGGCATCGACGTGTGGTACTCGCTGGATGCGGCTG |
| | | AACTGCTGGGCGCTGAAGCGGCCGACTACGAGAAGGTCACCGACATC |
| | | CTCGATGTCTGGTTCGATTCCGGCGTGACCCACGAAGCCGTGCTGGCT |
| | | GCCCGTGGCTTCGGCAAGCCGGCCGATCTGTACCTGGAAGGTTCGGA |
| | | CCAGCATCGCGGCTGGTTCCAGTCCTCGCTGCTGACCGGCGTGGCCAT |
| | | CGACAAGCGCGCGCCGTACAAGCAGTGCCTCACCCACGGTTTCACCG |
| | | TGGACGAGCACGGCCGCAAGATGTCCAAGTCGCTGGGCAACGGCATC |
| | | GAACCGCAGGAAATCATGAACAAGCTGGGCGCGGACATCCTGCGCCT |
| | | GTGGATCGCCTCGGCCGACTACAGCAACGAGATGTCGCTGTCGCAGG |
| | | AAATCCTCAAGCGCACCGCCGACGCCTACCGCCGCCTGCGCAACACC |
| | | GCCCGCTTCCTGCTGGGCAACCTGGACGGTTTCGATCCGGCCCAGCAC |
| | | CTGCGCCCGCTCAACGAGATGGTCGCGCTGGACCGCTGGATCGTGCA |
| | | TCGCGCCTGGGAGCTGCAGGAGAAGATCAAGGCGGCGTATGACAACT |
| | | ACGACATGGCCGAGATCGTGCAGTTGCTGCTGAACTTCTGCAGCGTG |
| | | GACCTGGGCTCGCTGTACCTGGACGTGACCAAGGATCGCCTGTATAC |
| | | GATGCCGACCGATTCGGATGGTCGTCGTTCGGCGCAGAGCGCGATGT |
| | | ACCACATCGCCGAAGCGTTCACCCGCTGGGTGGCGCCGATCCTGACC |
| | | TTCACCGCCGACGAGCTGTGGGGCTACCTGCCGGGCGATCGTGCCGG |
| | | CCACGTGCTGTTCACTACCTGGTACGAGGGCCTGGCACCGCTGCCGAC |
| | | CGATGCACAGCTCAACGCTGCCGACTTCGATCAGCTGCTGGCCGTGC |
| | | GCGAGCAGGTGGCCAAGGTGCTGGAGCCGATGCGCGCCAATGGTGCG |
| | | ATCGGTGCCGCGCTGGAAGCGGAGATCACCATCGCCGCCAGCGAAGA |
| | | GCAGGCCGCGCGCTGGCAGCCGCTGGCCGATGAACTGCGTTTCCTGTT |
| | | CATCAGTGGTGACGTGCAGGTGCGTCCGGCGACCACCGACGAGGTGT |
| | | TCGTCAGCGCGCAGCCGACGCAGAAGTCCAAGTGCGTGCGCTGCTGG |
| | | CACCACCGTGCCGACGTTGGCAGCAATGCCGACCACCCGGAACTGTG |
| | | CGGCCGCTGCGTGACCAACATCGCCGGTGCCGGCGAAGCGCGGAGCT |
| | | GGTTCTGA |
| 119 | DP69 Glycine | ATGAGCCACTTGTCTCCCCTGCTGATTGAACTGGGCACCGAAGAGTTG |
| | tRNA ligase beta | CCGGTCAAGGCGCTGCCGGGCCTGGCCCAGGCCTTCTTCGACGGTGTT |
| | subunit | GTCGATGGCCTGCGCAAGCGCGGCGTCGAACTGGAGCTGGGCGATGC |
| | | CCGCCCGCTGTCGACCCCGCGCCGCCTGGCCGTGCTGCTGCCGGGCGT |
| | | TGGCCTGGAACAGCCGGAACAACACAGCGAAGTGCTGGGCCCGTACC |
| | | TGAACATCGCGCTGGACGCCGAAGGCCAGCCGACCAAGGCGCTGCAG |
| | | GGTTTCGCGGCCAAGGCCGGGATCGACTGGACCGCGCTGGAGAAGAC |
| | | CACCGACAACAAGGGTGAGCGCTTCGTGCACCGTGCGGTGACTCCGG |
| | | GCGCGCGCACCGCTGCGCTGCTGCCGGAGATCCTGCGCGAGGCCATC |
| | | GCCGGCATGCCGATTCCCAAGCCGATGCGCTGGGGCGACCACAGCTG |
| | | GGGCTTCGCCCGCCCGGTGCACTGGCTGGTGCTGCTGCATGGCGGCG |
| | | ACGTGGTCGAGGCCGAACTGTTTGGCCTGAAGGCCGACCGCATGAGC |
| | | CGCGGCCACCGCTTCCTGCACGACAAGACCGTGTGGCTGACCCAGCC |
| | | GCAGGACTATGTCGAATCGCTGCGCGCCGCCTTCGTGCTGGTCGATCC |
| | | GGCCGAGCGCCGCCGGCGCATCGTTGCCGAAGTGGAAGCCGCTGCCG |
| | | CCACCGCCGGTGGCAGCGCACGCATCACCGAGGACAACCTGGAGCAG |
| | | GTGGTGAACCTGGTCGAGTGGCCGGCGGCAGTGTTGTGCAGCTTCGA |
| | | GCGCGCGTTCCTGGCGGTACCGCAGGAAGCGCTGATCGAGACGATGG |
| | | AGATCAACCAGAAGTTCTTCCCGGTGCTGGATGACGGCGGCAAGCTG |
| | | ACCGAGAAGTTCATCGGCATCGCCAACATCGAGTCCAAGGACGTGGC |
| | | CGAAGTGGCCAAGGGCTACGAGCGCGTGATCCGCCCGCGCTTCGCCG |
| | | ATGCCAAGTTCTTCTTCGACGAAGACCTGAAGCAGGGCCTGCAGGCG |
| | | ATGGGCGAGGGCCTGAAGACGGTGACCTACCAGGCCAAGCTGGGCA |
| | | GCGTGGCCGACAAGGTCGCGCGCGTGGCGGCGCTGGCCGAGGTGATC |
| | | GCTGCGCAGGTGGGGGCCGACCCGGTGCTGGCCAAGCGTGCCGCGCA |
| | | GCTGGCCAAGAACGACCTGCAGTCGCGCATGGTCAATGAGTTCCCGG |
| | | AACTGCAGGGCATCGCTGGCCGCCACTACGCGGTGGCCGGTGGCGAG |
| | | TCGCCGGAGGTGGCGCTGGCCATCGACGAGGCCTACCAGCCGCGCTT |
| | | CGGTGGCGATGACATCGCGCTGTCGCCGCTGGGCAAGGTGCTGGCGA |
| | | TCGCCGAGCGTGTGGACACGCTGGCCGGCGGTTTCGCCGCGGGCCTG |
| | | AAGCCGACCGGCAACAAGGACCCGTTCGCCCTGCGCCGCAACGCGCT |
| | | GGGCCTGGCCCGCACGATTATCGAAAGTGGCTTCGAGCTGGACCTGC |
| | | GCGCGCTGCTGGCCAGCGCCAATGCCGGGCTGACCGTGCGCAACGTG |
| | | CAGGCCGACGTGGCTGAGCTGTACGACTTCATCCTCGACCGCCTGAA |
| | | GGGCTACTACAGCGACAAGGGCGTGCCGGCCAGCCACTTCAATGCGG |
| | | TGGCTGAGCTGAAGCCGGTCTCGCTGTACGATTTCGACCGTCGCCTGG |
| | | ACGCCATCGGTATCTTCGCGGCGCTGCCGGAGGCCGAGGCGCTGGCA |
| | | GCGGCCAACAAGCGCATCCGCAACATCCTGCGCAAGGCCGAAGGCGA |
| | | TATTCCGGGCCAGATCGATGCGGCCCTGTTGCAGGAAGATGCCGAGC |
| | | GCGCGCTGGCGGAAGCCGTGACTGCAGCCATCGACGACACCGGCGCC |
| | | AGCCTGCACCAGAAGGACTACGTGGCCGTGCTGGCGCGCCTGGCCCG |
| | | CCTGCGTCCGCAGGTCGATGCGTTCTTCGATGGGGTGATGGTCAATGC |
| | | CGAGGATCCGGCACTGCGCGGCAACCGCCTGGCGCTGCTGACGATGC |
| | | TGGGCGAGCGCTTGGGCAAGGTCGCGGCGATCGAGCATCTGTCGAGC |
| | | TGA |
| 120 | DP69 Glutamine | ATGTCCGTGGAAACCGTAGAGAAGCTGATCAAGGACAACCAGATCGA |
| | synthetase | GTTCGTCGATCTGCGCTTCGTCGACATGCGTGGTGTCGAACAGCATGT |
| | | GACCTTCCCGGTCAGCATCGTCGAGCCGTCGCTGTTTGAAGAAGGCA |
| | | AGATGTTCGATGGCAGCTCGATCGCCGGCTGGAAGGGCATCAACGAG |
| | | TCGGACATGGTGCTGCTGCCGGACACCGCCAGCGCCTACGTCGACCC |
| | | GTTCTACGCCGATCCGACCATCGTGATCAGCTGCGACATCCTCGACCC |
| | | GGCCACCATGCAGCCGTATGGCCGTTGCCCGCGCGGCATCGCCAAGC |
| | | GCGCCGAGTCCTACCTGAAGTCCTCGGGCATCGCCGAAACCGCGTTCT |
| | | TCGGCCCGGAGCCGGAGTTCTTCATCTTCGACTCGGTGCGTTTCGCCA |
| | | ATGAAATGGGCAACACCTTCTTCAAGGTCGACTCGGAAGAAGCGGCG |
| | | TGGAACAGCGGCGCCAAGTACGACGGCGCCAACAGCGGCTACCGTCC |
| | | GGGCGTGAAGGGCGGTTATTTCCCCGTTCCGCCGACCGACACCCTGC |
| | | ACGACCTGCGTGCGGAGATGTGCAAGACCCTGGAACAGGTCGGCATC |
| | | GAAGTGGAAGTGCAGCACCACGAAGTGGCCACCGCCGGCCAGTGCG |
| | | AGATCGGCACCAAGTTCAGCACCCTGGTGCAGAAGGCCGACGAACTG |
| | | CTGCGGATGAAGTACGTCATCAAGAACGTCGCCCACCGCAACGGCAA |
| | | GACCGTCACCTTCATGCCCAAGCCGATCGTCGGCGACAACGGCAGCG |
| | | GCATGCACGTGCACCAGTCGCTGTCCAAGGGCGGCACCAACCTGTTC |
| | | TCCGGTGACGGCTACGGTGGCCTGAGCCAGATGGCGCTGTGGTACAT |
| | | CGGCGGCATCTTCAAGCATGCCAAGGCGATCAACGCCTTTGCCAACT |
| | | CGGGTACCAACAGCTACAAGCGCCTGGTGCCGGGCTTCGAAGCCCCG |
| | | GTGATGCTGGCCTACTCGGCGCGCAACCGTTCGGCCTCGTGCCGCATT |
| | | CCGTGGGTGTCCAACCCGAAGGCGCGTCGCATTGAAATGCGCTTCCC |
| | | CGATCCGATCCAGTCGGGCTACCTGACCTTCACCGCGCTGATGATGGC |
| | | CGGCCTGGACGGCATCAAGAACCAGATCGACCCGGGCGCACCGAGCG |
| | | ACAAGGATCTGTACGACCTGCCGCCGGAAGAAGAGAAGCTGATTCCG |
| | | CAGGTCTGCTCCTCGCTGGACCAGGCCCTGGAAGCGCTGGACAAGGA |
| | | CCGTGAGTTCCTCAAGGCCGGTGGCGTGATGAGCGATGACTTCATCG |
| | | ACGGCTACATCGCGCTGAAGATGCAGGAAGTGACCAAGTTCCGCGCG |
| | | GCGACCCACCCGCTGGAATACCAGTTGTACTACGCCAGCTGA |
| 121 | DP69 Glucose-6- | ATGACAACGAACAACGGATTCGACTCGCTGCATTCCCACGCCCAGCG |
| | phosphate | CCTGAAGGGCGCAAGCATCCCCAGCCTGCTCGCCGCCGAACCCGGCC |
| | isomerase | GCGTACAGGACCTGGCGCTGCGGGTCGGTCCGTTGTATGTCAACTTCG |
| | | CCCGGCAGAAATACGATGCCGCGGCGTTGCAGGCGCTGTTGGCGCTG |
| | | GCTGCCGAACGTGATGTCGGCGGCGCCATCACGCGCCTGTTCCGTGG |
| | | CGAGCAGGTCAATCTGACCGAAGGCCGCGCCGCACTGCACACCGCAC |
| | | TGCGCGGCGACGTGGTCGATGCGCCGGTTGCCGCCGAGGCCTATGCC |
| | | ACGGCCCGCGAAATCCGCCAGCGCATGGGCGTGCTGGTGCGCGCACT |
| | | GGAAGACAGTGGCGTGACCGATGTGGTCAGTGTCGGCATCGGCGGTT |
| | | CCGATCTCGGTCCGCGTCTGGTCGCCGACGCACTGCGTCCAGTCACTG |
| | | GCGCTCGCCTGCGCGTGCATTTCGTGTCTAACGTGGACGGCGCTGCCA |
| | | TGCAGCGCACGCTGGCCACGCTGGATCCGGCGAAGACCGCCGGCATC |
| | | CTCATTTCCAAGACCTTCGGTACCCAGGAAACCCTGCTCAACGGCCAG |
| | | ATCCTGCACGATTGGCTGGGTGGCAGCGAGCGCCTGTACGCGGTCAG |
| | | CGCCAATCCGGAACGCGCCGCCAAGGCCTTCGCCATCGCCGCCGAGC |
| | | GCGTGCTGCCGATGTGGGACTGGGTAGGGGGGCGCTATTCGCTGTGG |
| | | TCGGCCGTCGGTTTCCCGATCGCACTGGCCATCGGCTTCGAGCGTTTC |
| | | GAGCAGTTGCTGGAAGGCGCCGCGCAGATGGATGCGCATGCGCTGGA |
| | | CGCGCCGCTGGAGCGCAACCTGCCGGTGCTGCACGGCCTGACCGACA |
| | | TCTGGAACCGCAATCTGCTGGGCTCTGCCACGCATGCGGTGATGACCT |
| | | ACGACCAGCGCTTGGCGCTGCTGCCGGCCTACCTGCAGCAGCTGGTG |
| | | ATGGAAAGCCTGGGCAAGCGCGTGCAGCGCGATGGCCAGCCGGTCAC |
| | | CACCGACACCGTGCCGGTGTGGTGGGGCGGTGCCGGCACCGATGTGC |
| | | AGCACAGCTTCTTCCAGGCCCTGCACCAGGGCACCAGCATCATTCCG |
| | | GCCGATTTCATCGGCTGCGTGCACAACGACGATCCGTATACGGTCAA |
| | | CCACCAGGCGTTGATGGCCAACCTGCTGGCGCAGACCGAAGCGCTGG |
| | | CCAACGGCCAGGGCAGTGACGATCCGCACCGCGATTATCCGGGTGGC |
| | | CGCCCGAGCACGATGATCCTGCTCGACGCGCTCACCCCGCAGGCGCT |
| | | GGGCGCCTTGATCGCGATGTACGAACACGCCGTGTACGTGCAGTCGG |
| | | TGATCTGGAACATCAACGCCTTCGACCAGTTCGGTGTCGAGCTGGGC |
| | | AAGCAGCTGGCCAGTGGCCTGCTGCCCGCTCTGCAGGGTGAGGATGT |
| | | CGAGGTCAACGACCCGCTGACCCGTGAGCTGCTGGCCCAGCTGAAGG |
| | | GCTGA |
| 122 | DP69 Leucine | ATGACCAGCGTCGAACCCAACGTTTACGATCCGCAGCAGGTTGAATC |
| | tRNA ligase | CGCCGCCCAGAAGTACTGGGACGCTACCCGTGCCTTCGAGGTCGATG |
| | | AAGCCTCGGACAAGCCGAAGTACTACTGCCTGTCGATGCTTCCGTATC |
| | | CGTCCGGTGCGCTGCACATGGGCCACGTGCGCAATTACACGATCGGC |
| | | GACGTGATCAGCCGCTACAAGCGCATGACCGGCCACAACGTGCTGCA |
| | | GCCGATGGGCTGGGACGCGTTTGGCCTGCCGGCGGAAAACGCTGCGA |
| | | TCAAGAACAAGACCGCGCCGGCCGCCTGGACCTACAAGAACATCGAC |
| | | CACATGCGCAGCCAGCTGCAGTCGCTGGGCTATGCCATCGACTGGTC |
| | | GCGCGAGTTCGCCACCTGCCGCCCGGACTATTACGTCCACGAGCAGC |
| | | GCATGTTCACCCGCCTGATGCGCAAGGGCCTGGCCTACCGCCGCAAC |
| | | GCGGTGGTGAACTGGGACCCGGTCGACCAGACCGTGCTGGCCAACGA |
| | | GCAGGTCATCGACGGCCGTGGCTGGCGCTCCGGCGCGCTTGTGGAAA |
| | | AGCGCGAGATCCCGCAGTGGTTCCTGCGCATCACCGACTACGCCCAG |
| | | GAACTGCTGGACGGCCTGGATGAGCTGGACGGCTGGCCGGAGTCGGT |
| | | CAAGACCATGCAGCGCAACTGGATCGGCCGCTCCGAAGGGCTGGAAA |
| | | TCCAGTTCGACGTGCGCGACGTCGATGGTGCCGCACTGGATCCGCTGC |
| | | GCGTGTTCACCACCCGCCCGGACACCGTGATGGGCGTGACTTTCGTGT |
| | | CGATCGCGGCCGAACATCCGCTGGCGCTGCATGCCGCGAAGAACAAC |
| | | CCGGAACTGGCTGCGCTGCTGTCGGAAATGAAGCAGGGCGGCGTGTC |
| | | CGAGGCCGAGCTGGAGACCCAGGAAAAGCGCGGCATGGATACCGGC |
| | | CTGCGCGCCGTGCATCCGGTTACCGGTGCCCAGGTGCCGGTGTGGGTC |
| | | GCCAACTTCGTGCTGATGGGCTACGGCACTGGCGCGGTGATGGCCGT |
| | | ACCGGGCCACGACCAGCGCGACAATGAATTCGCCAACAAGTACAACC |
| | | TGCCGATCCGCCAGGTCATCGCGCTGAAGTCGCTGCGCAAGGACGAA |
| | | GGCGCCTACGACGCGACGCGCTGGCAGGACTGGTACGGCGACAAGAC |
| | | CCGCGAGACCGAACTGGTCAACTCCGAAGAGTTCGACGGCCTGGACT |
| | | TCCAGGGCGCTTTCGAGGCGCTGGCCGAACGGTTCGAGCGCAAGGCC |
| | | CAGGGACAGCGCCGGGTGAACTACCGCCTGCGCGACTGGGGCGTGAG |
| | | CCGCCAGCGCTACTGGGGCTGCCCGATTCCGGTGATCTACTGCGACA |
| | | AGTGTGGCGCGGTACCGGTGCCGGAAGACCAGCTGCCGGTGGTGCTG |
| | | CCGGAAGACGTGGCGTTCGCCGGTACCGGTTCGCCGATCAAGACCGA |
| | | TCCGGAATGGCGCAAGACCACCTGCCCGGACTGCGGCGGTGCGGCCG |
| | | AGCGTGAGACCGACACCTTCGACACCTTCATGGAGTCGAGCTGGTAC |
| | | TACGCCCGCTACACCTCGCCGGGCGCCCGCGATGCGGTCGACAAGCG |
| | | CGGCAACTACTGGCTGCCGGTGGACCAGTACATCGGTGGCATCGAAC |
| | | ACGCGATCCTGCACCTGATGTATTTCCGCTTCTACCACAAGCTGCTGC |
| | | GCGACGCGCGGATGGTGGACAGCAACGAACCCGCGCGGAACCTGCTG |
| | | TGCCAGGGCATGGTGATCGCTGAGACCTACTACCGCCCGAACCCGGA |
| | | CGGCTCGAAGGACTGGATCAACCCGGCCGATGTGGAAGTGCAGCGCG |
| | | ACGAGCGCGGCCGCATCACCGGCGCCACCCTGATCGCCGACGGTCAG |
| | | CCGGTGGTGGTCGGTGGTACCGAGAAGATGTCCAAGTCGAAGAACAA |
| | | CGGCGTGGACCCGCAGGCGATGGTCGGCAAGTACGGCGCCGATACCG |
| | | TGCGCCTGTTCTCGATGTTCGCTGCACCGCCGGAACAGTCGCTGGAAT |
| | | GGAACGAAGCCGGCGTGGACGGCATGGCCCGCTTCCTGCGCCGCCTG |
| | | TGGGCACAGGTGCAGAAGCACGCTGCCGAGGGTGCCGCACCGGCGCT |
| | | CGACGCGGCCGCGCTGGATGCCGGCCAGAAGGCCCTGCGCCGCAAGA |
| | | CCCACGAGACCATCGGCAAGGTCGGCGACGACTACGGCCGCCGCCAC |
| | | AGCTTCAACACCGCCATTGCCGCGGTGATGGAGCTGATGAACGCGCT |
| | | GGCCAAGTTCGAGGACGGCAGTGAACAGGGGCGCGCCGTGCGCCAG |
| | | GAAGCACTGCAGGCCATCGTGCTGCTGCTCAACCCGATCACCCCGCA |
| | | TGCCAGCCACGCCCTGTGGCAGGTACTGGGCCATGGCGAAACGCTGC |
| | | TGGAAGATCAGCCGTTCCCGCAGGCCGACAGCAGTGCGCTGGTGCGC |
| | | GATGCGCTGACTTTGGCCGTGCAGGTCAATGGCAAGCTGCGTGGCAC |
| | | CATCGAGGTCGCCGCCGATGCCGCGCGCGAGCAGATCGAAGCGCTGG |
| | | CCCTGGCCGAGCCGAACGCGGCCAAGTTCCTGGAAGGCCTGACGGTG |
| | | CGCAAGATCATCATCGTTCCCGGCAAGATCGTGAACATCGTCGCTGCC |
| | | TGA |
| 123 | DP70 Glycine | ATGTCTAAACATACAGTATTGTTCGAATTGGGCTGTGAAGAACTTCCA |
| | tRNA ligase beta | CCTAAAAGCCTCAAAAAATTACGTGATGCACTGCATGCTGAAACGGT |
| | subunit | AAAAGGCTTAAAAGATGCAGGCTTAGCATTCGACTCAATCGAAGCTT |
| | | ATGCAGCACCGCGTCGTTTGGCACTTAAAATTGTGAATATCGATGGCG |
| | | CTCAGCCTGATACACAAAAACGCTTTGACGGCCCTGCAAAAGAAGCG |
| | | GCTTATGATGCTGAAGGCAAACCAAGCAAAGCATTAGAAGGCTTTAT |
| | | GCGTGGTCAAGGCATCACTGCGGATCAAGTCACCACGTTCCAAGCGG |
| | | GTAAAGTTGAAAAGGTTTGCTATTTAAAAGATGTTAAAGGTCAAAGC |
| | | CTTGAGGTTTTACTGCCACAAATTCTACAAGCAGCTTTGGACAATCTT |
| | | CCAATTGCAAAACGTATGCGTTCAGCGGCAAGCCGTACTGAATTCGT |
| | | GCGTCCTGTAAAATGGGTGGTGTTGCTCAAAGACAATGATGTGATTG |
| | | CAGCCACTATTCAAGATCACAAAGCAGGCAATGTGACTTATGGTCAT |
| | | CGTTTCCATGCCCCTGAAGCGATTACTTTGGCTCATGCAGATGAATAT |
| | | CTTGCCAAGTTAAAAGCGGCTTATGTGGTTGCTGACTTTGCAGAACGC |
| | | CAAGCCATCATTGACCAACAAGTCAAAGCGTTGGCTGATGAAGTTAA |
| | | TGCGATTGCGATTGTACCAAGCGACCTGCGTGATGAAGTGACCGCAT |
| | | TGGTGGAATGGCCTGTTGCGCTACGTGCCAGCTTTGAGGAGCGTTTCC |
| | | TTGCTGTACCGCAAGAAGCTTTGATTACCACGATGCAAGACAACCAA |
| | | AAATACTTCTGTTTGGTGAATAGTGATAACAAGCTACAGCCTTATTTC |
| | | ATTACTGTTTCAAATATTGAGTCTAAAGATCCGATTCAAATTATTGAA |
| | | GGCAATGAAAAAGTGGTTCGTCCACGTTTGTCGGATGCTGAATTCTTC |
| | | TTCTTGCAAGATCAAAAGCAACCACTAGCTTCTCGTAAAGAAAAACT |
| | | GGCTAACATGGTGTTCCAAGCACAATTGGGTACGCTGTGGGATAAGT |
| | | CACAACGTATTGCAAAATTGGCTGTGGCTTTATCGAACATCACGGGTG |
| | | CAACTGCGGCTGATGCTGAAAAAGCAGCATTGCTGGCAAAATGTGAC |
| | | TTAACCTCTGAATTGGTGGGTGAATTCCCTGAACTTCAAGGCATTGCG |
| | | GGAACCTATTACGCACGCATTGAAGGTGAAAACCATGAAGTGGCTGA |
| | | AGCTTTAGGCGAACAGTATTTACCTAAATTTGCAGGCGATGTTTTACC |
| | | GCAAACAAAAACAGGCACAACCATTGCCCTTGCCGACCGTTTAGACA |
| | | CGCTCACGGGTATTTTTGGTATTGGTCAAGCACCTACAGGTTCTAAAG |
| | | ATCCGTTTGCATTACGTCGTTCTGCAATCGGTATTTTACGTTTGGTGAC |
| | | TGAAAACAATCTTGATGTGTCGATTGAAGATTTAATCCAGCTGGCATT |
| | | AAACGCTTATGGCGATGTTGTAGCGGATCATGCGAAGACTTTAGCGG |
| | | ATGCTGTTGCATTCCTTGAAGGTCGTTACCGTGCCAAGTATGAAGACC |
| | | AAGGCGTTGCAGTTGATGTGATTCAAGCGGTTCAAGCATTATCACCA |
| | | AAATCACCTTTAGATTTTGATAAGCGTGTGACTGCGGTAAATCATTTC |
| | | CGTGCATTGCCTGAAGCTGCTGCACTGGCTGCTGCAAATAAGCGTGTT |
| | | GCCAACATTCTTGCCAAAGAAGCAGAACTAACAGGCGCAGTGGTTGA |
| | | AGCAAACTTGGTTGAAGAGGCTGAAAAAGCATTATTCGCTGTACTTG |
| | | CTAAAATTACGCCTGAAGTTGAACCATTATTTGCTGCCAAAGATTACA |
| | | CCACTGCATTGTCTAAGCTTGCTGCTTTACGTGCGCCTGTGGATGCAT |
| | | TCTTTGAAGGCGTCATGGTCATGGCAGATGATGCAGAATTGAAAGCC |
| | | AACCGTTTACGTTTATTGGCTCAATTACGTGGTTTGTTTACAAGTGTTG |
| | | CGGATATTTCGGTGTTGCAGCACTAA |
| 124 | DP70 DNA | ATGAGTTCAGAAGATCAAGCTGCTTCTCAAACAGAACAAACCAATGA |
| | gyrase subunit B | AAAGGCTTATGATTCCTCTAGTATCAAAGTATTACGTGGCCTAGATGC |
| | | TGTTCGTAAGCGTCCGGGTATGTATATTGGTGATACGGACGATGGTTC |
| | | AGGTTTACATCACATGGTGTTTGAGGTGGTCGATAATGCGATTGATGA |
| | | AGCCTTAGCGGGTCACTGTGATGAAATCTTAGTCACCATCCATGAAG |
| | | ATGAGTCTGTAAGTGTTGCAGATAACGGTCGTGGGATTCCAACGGAT |
| | | ATTCACCCTGAAGAAGGGGTATCTGCCGCTGAAGTGATTTTAACCATT |
| | | TTGCATGCTGGCGGTAAGTTTGATGATAATAGCTATAAAGTTTCCGGT |
| | | GGTTTACACGGGGTAGGTGTTTCTGTTGTAAATGCCTTGTCGAGTAAA |
| | | TTATTACTAAATATTCGTCGTGCAGGAAAAGTATATGAACAGGAATA |
| | | TCACCATGGTGATCCTGTCTATCCATTACGCGCGATTGGTGATACTGA |
| | | AGAAACCGGTACCACCGTTCGTTTCTATCCGAGTGAATTAACCTTCTC |
| | | TCAAACGATTTTTAATGTTGATATTTTAGCGCGTCGTTTGCGCGAACT |
| | | TTCATTCTTAAATGCAGGGGTTCGTATTGTATTACGTGATGAACGTAT |
| | | CAATGCTGAACATGTATTTGATTATGAAGGTGGTTTGTCTGAATTTGT |
| | | AAAATATATCAATCAAGGTAAAACCCACTTGAATGAGATTTTTCATTT |
| | | TACCAGTGAAGTTGTGGAAACAGGAATTACTGTTGAAGTAGCATTAC |
| | | AGTGGAATGATACTTATCAAGAAAATGTCCGTTGCTTTACCAATAACA |
| | | TCCCACAAAAAGATGGTGGTACGCATTTAGCCGGTTTCCGTGCCGCGT |
| | | TAACACGGGGTTTAAACCAGTATCTTGATAGTGAAAATATTCTTAAGA |
| | | AAGAAAAAGTTGCTGTCACAGGTGATGATGCCCGTGAAGGTTTAACG |
| | | GCGATTGTTTCAGTGAAAGTGCCTGATCCAAAATTCTCATCACAAACC |
| | | AAAGAAAAATTGGTTTCCAGTGAAGTGAAAACTGCTGTAGAGCAGGC |
| | | GATGAACAAGTCTTTTTCTGAATATCTTTTAGAAAATCCACAAGCGGC |
| | | TAAATCGATTGCCGGCAAAATTATTGATGCTGCACGTGCACGTGATGC |
| | | TGCGCGTAAAGCACGTGAAATGACACGTCGTAAGAGTGCATTAGATA |
| | | TTGCTGGTCTGCCTGGTAAACTGGCGGATTGCCAAGAAAAAGATCCA |
| | | GCATTGTCTGAACTTTACTTGGTCGAAGGTGACTCGGCGGGCGGTTCT |
| | | GCAAAACAGGGTCGTAACCGTAAGATGCAAGCTATTCTGCCGCTTAA |
| | | AGGTAAAATCTTAAACGTAGAACGTGCACGTTTTGACAAAATGATTT |
| | | CATCGCAAGAAGTGGGCACGCTGATTACTGCACTGGGCTGTGGTATT |
| | | GGTCGTGAGGAATACAATCCTGATAAATTGCGTTATCACAAAATCATT |
| | | ATCATGACCGATGCCGACGTCGATGGTTCGCACATTCGTACGCTCCTG |
| | | TTGACCTTCTTCTTCCGTCAAATGCCAGAACTTGTGGAACGTGGTTAT |
| | | ATTTATATTGCACAGCCACCGTTGTATAAGTTGAAAAAAGGTAAGCA |
| | | AGAGCAATATCTTAAAGATAATGATGCTTTAGAAACCTATCTTATTTC |
| | | GAATGCCATTGATGAGCTTGAACTGCATATTAGTGCTGAGGCACCTGC |
| | | GATTCGTGGTGAATCTTTGGCTAAAGTGATTGCTGATTATCAAACCTC |
| | | ACAAAAAAGTTTAAATCGTTTAACGCTACGTTATCCTGCAAGCTTGCT |
| | | GGATGGTTTACTTGGTTTGGATGCATTTAAACTTGATCAAAATCATGA |
| | | TGAAGATTATGTAAAACAATGGTCTGAACAATTGCGTGCAGCAATTG |
| | | AACAACACCAACCAAGTTTGCGTCCTGAAATCACCTTAGAAGCTTTTG |
| | | AAAAAGAGCATGCAGATGGTGAGAAAGTGACGCATTATTGGCCACGT |
| | | GTAACGGTCTATGTACATAACTTGCCGCATCATTATTTACTTGATTCT |
| | | GGATTATTGGCTTCAAGTGAATACAAGCGTTTACTGCAAAATTCGAA |
| | | GAGTTGGTTCACATTGCTTGAAGATGGCGCTTATTTGCAAAAAGGTGA |
| | | GCGTAAAATTCATGTCGCCACTTTCCATCAAGTTTGGCAACATATTTT |
| | | ATCCGACTCGCGTCGTGGCATGATGATCCAGCGCTATAAAGGTTTGG |
| | | GTGAGATGAACGCGGAACAGCTTTGGGAAACCACCATGGATCCTGAA |
| | | AACCGTAACATGTTGCAAGTCACCATTAATGATGCGATTGAAGCGGA |
| | | TCGTATGTTCTCTTGTTTGATGGGAGATGATGTGGAACCACGTCGTGC |
| | | CTTCATTGAAGAAAATGCTTTAAATGCGGATATTGACGCTTAA |
| 125 | DP70 Leucine | ATGACTACTTCTCACATTGACCCTGAATATCAAGCGAGCGCGATTGAA |
| | tRNA ligase | TCCACTGTCCAACAAGACTGGGAAACTCGCAAAGCCTTTAAAGTTGC |
| | | CGACACTGTAGAAGGTAAACATCGTTATATCCTCTCGATGTTCCCTTA |
| | | TCCAAGTGGCAAGCTGCATATGGGTCATGTGCGTAACTACACCATTG |
| | | GCGACGTGATTAGCCGTTTCCACCGTCTCAAAGGTGAAACTGTCCTAC |
| | | AACCGATGGGTTGGGATGCTTTTGGTCTGCCTGCGGAAAATGCAGCG |
| | | ATTGCACACCAAGTTGCCCCTGCAAAATGGACCTTTGAAAACATCGC |
| | | GTACATGCGTGACCAGTTAAAAAAATTGGGTCTGTCAGTCGATTGGG |
| | | ATCGTGAATTTGCGACCTGTACGCCAGAGTATTATCACTGGGAACAAT |
| | | GGTTATTTGTACAGCTGTATAAAAAAGGGCTGATTTATCGCAAACTTT |
| | | CAACGGTAAACTGGGATCCTGTCGATCAGACTGTACTTGCTAATGAA |
| | | CAAGTTGAAAATGGTCGTGGTTGGCGTTCGGGTGCATTGGTTGAAAA |
| | | ACGTGATATTCCAATGTATTACTTCCGTATTACCGATTATGCACAAGA |
| | | ATTATTAGACGATTTAGATTCGCTTAAAGATGGTTGGCCGCAACAAGT |
| | | CTTGACCATGCAACGCAACTGGATTGGTCGTTCACAAGGCATGGAAA |
| | | TCACCTTTCCATCTGCGAACCCTGAAATCTATGCAGATGATTTAACGG |
| | | TTTATACCACACGTGGTGACACCTTGATGGGCGTGACGTATGTTGCGG |
| | | TTGCCGCTGAACATCCAATGGCGCTTAAAGCGGCTGAAACAAATCCC |
| | | GAATTGGCTGCATTTATTGAAGAATGCCGTATGGGTTCAGTGGCTGAA |
| | | GCAGATCTTGCCACTGCCGAGAAAAAAGGCATGGCCACTGGTTTGTC |
| | | TGTGAAGCATCCTGTAACGGGTGAAGTGGTTCCAGTGTGGATTGCGA |
| | | ACTATGTATTGATGTCATACGGTTCAGGTGCGGTGATGGCAGTTCCAG |
| | | CACACGACGAACGTGATTTCGAATTTGCCAACAAATATGGTTTAACCC |
| | | TCCAGCAAGTGATTGATGCCAAAGGTGCAGACGATGCTGAATTTTCT |
| | | GCAACTGAATGGCAGGAATGGTATGGCTCGAAAGAAGGCAAACTGGT |
| | | TAATTCTGGCGAATTTGACGGTTTAGACTTCCAAGCTGCATTTGATGC |
| | | ATTCATTGCAAAATTAGAACCACAAAAACTGGCAAATACGAAAGTTC |
| | | AGTTCCGTCTACGTGACTGGGGTGTTTCGCGTCAGCGTTATTGGGGTT |
| | | GTCCAATTCCAATGATCAACTGTGAAACTTGTGGTCAAGTACCTGTAC |
| | | CTGAAGAACAACTTCCAGTAATTTTACCAACTGACGTGGTGCCAGAT |
| | | GGTTCAGGCAATCCGTTAAATAAAATGCCTGAATTTTATGAAACCCA |
| | | ATGTCCATGTTGTGGTGCAGGTGCACGCCGTGAAACCGATACTTTGGA |
| | | TACGTTCGTAGAGTCATCTTGGTACTATGCACGTTATGCATCTCCAGA |
| | | TTTCACTGGCGGTTTAGTTAAACCTGAAGCTGCAAAATCATGGCTACC |
| | | AGTCAACCAATATATTGGCGGTGTGGAACATGCAATTTTGCATTTATT |
| | | GTATGCCCGTTTCTTCCATAAATTGATGCGTGATGAAGGCGTCGTTGA |
| | | AGGCAATGAACCTTTCGCTAACTTACTGACTCAAGGTATGGTTTTAGC |
| | | TGATACCTTCTACCGTGAAGCCGAATCAGGTAAGAAAACATGGTTTA |
| | | ATCCTGCGGATATTGAATTAGAAAAAGACGAAAAAGGTCGTGTTCTT |
| | | TCTGCTAAATACACAGGTGATGGCCAAGAAGTTGTGGTTGGCGGTCA |
| | | AGAAAAAATGTCGAAATCGAAAAATAATGGCATCGACCCGCAATCGA |
| | | TTATTGATCAATACGGCGCAGATACTGCACGTGTATTTATGATGTTTG |
| | | CGGCCCCACCCGATCAATCGCTTGAATGGTCTGATGCCGGTGTGGAA |
| | | GGTGCAAACCGTTTCTTGAAACGTGTATGGCGTTTAACCACAGGTTTC |
| | | TTAGAAAAAGGCAACCATGCTGCTGTAATTGATGTTGCGAATTTGTCA |
| | | TCAGCGGCACAAGACTTACGTCGTAAAACCCACGAAACCATTCAAAA |
| | | AGTCGGTGATGACATTGAACGTCGTCATGCCTTCAATACTGCCATTGC |
| | | AGCGCAAATGGAATTATTGAATGCTTGCAATAAATTTGAAGCCAAAG |
| | | ATGATAATGACGTTGCGGTTGAACGCGATGCTATTGTTAGCTTACTCA |
| | | CTTTACTTGCACCATTTGCACCACATTTAAGTCAGACCCTATTGGCTC |
| | | AATTCGGTATTGAGTTAACTGAAACCTTGTTCCCTACTGTGGATGAGT |
| | | CTGCGCTAACCCGCAACACACAAACTATTGTGGTACAGGTCAATGGT |
| | | AAACTTCGTGGCAAGTTGGAAGTGTCTGTTGATCTCTCTAAAGAAGAT |
| | | ATTTTGGATCAAGCCAAAGCATTGCCTGAAGTACAACAATTCTTAACC |
| | | GGTCCAACCAAGAAAGAAATTGTGGTGCCGAATAAATTGGTCAATTT |
| | | GGTGGTTTAA |
| 126 | DP70 Glucose-6- | ATGAATAGTATTGAAAAATTTCCCTTGCATGATACGGATCTGATTCAG |
| | phosphate | GAAAAACTAAAAAGTTTTGCCCAACAAGAGCAAGAGATTAATTTAAA |
| | isomerase | TTATTTATTTAAAAAAAATAAAAAACGTTTTGATGAATATTCCGTTCA |
| | | TGCGGGTCAGTTATGTTTTGATTATAGTAAGCACCGTGTTGATGAGCG |
| | | TATTATTAACGAGCTTATTTGTTATGCGGAATCACAACATTTGGGTAA |
| | | CTGGATTCAGCGCTTATTTTCTTTAGAAAAAATTAATTACACTGAAAA |
| | | TCGCGCAGCGATGCATTGGGCTTTGCGTTTGCCGAAGCAAGATAGTA |
| | | CACATGCAGATTTGGCAGCGCAGGTACATAGTCAGCTTGATCGTATGT |
| | | ATCAATTGGTCGAGAAAATTCATCAGGGGCAGTATCGAGGAGCTACA |
| | | GGTGAGGTCATCCATGATGTGGTCAATATTGGTGTCGGTGGATCAGAT |
| | | CTTGGTCCTTTAATGGTGTCTCAAGCGCTGACTGATTTTAAAGTTCAA |
| | | ACGGCTCAAAAATTAAAAGTCCATTTTGTTTCGACGATGGATGGCAG |
| | | CCAACTTTCAGATCTTTTACATCAGTTTCGCCCAGAAACCACCTTGTTT |
| | | ATTATTTCATCCAAGTCTTTTGGCACCATTGATACGCTTTCCAATGCAC |
| | | AAACGGCAAAATGCTGGCTTGAGCAATCTTTAGGAACGTCGAAATCA |
| | | GTTCTAAGATGTCACTTTGTTGGTGTTTCAACCAAGCCCGATAAGATG |
| | | ACCGAGTGGGGAATCAGCACTGAAAATCAATTCTTATTGTGGGATTG |
| | | GGTCGGTGGGCGCTATTCACTATGGTCGTGTATTGGTTTGCCTATTGC |
| | | ATTAAGTATTGGGGTCGAGGGCTTTAAACAGTTGCTTGCTGGTGCTTA |
| | | TGAAATGGATCAGCATTTTCAGAACACACCACTTGAACAAAATATTC |
| | | CTGTGTTGATGGGTTTACTGGGAATATGGAATAACAACTTCCTGAATA |
| | | TTCAAACTCATGCGGTACTTCCTTATGATGGTCGGCTGAAATATTTTG |
| | | CGGCTTATTTACAGCAATTGGAAATGGAGTCGAATGGTAAGTCGATT |
| | | CAGCGTTCTGGTGAAAAAGTCGTATTAGATACCTGCCCAATTTTATGG |
| | | GGTGAAGTTGGACCAAATGCACAACATGCTTTTTATCAGCTGCTGCAT |
| | | CAAGGTACACATGCTGTGAGTTGTGACTTTATTGCACCTGTGAAACGC |
| | | TATAATGCCAATCAATTTACCTATGTTGAAAATGCAGAGGCTTTAGTT |
| | | GAACAACACCATTTAGCCTTATCGAATTGTTTGGCACAATCACGTCTA |
| | | TTGGCCTTTGGTAATCATGTTCTAGATCCGAAAGAAGTAGAAAGTTCA |
| | | CCGAAATATAAACAATATGCAGGCAACCAACCGACCACAACAATTTT |
| | | GTTAAAAGAGTTGAATCCGCGCAGTTTAGGTATGCTCATTGCGATGTA |
| | | TGAGCACAAGGTATTTGTGCAATCCGTGATGTGGAATATTAATCCATT |
| | | TGACCAATGGGGCGTAGAAAAAGGTAAAGAAATTGCCAATCAACTGT |
| | | TACCGATTCTCAATCAAGAGCAAGCTGATGTTTCTGATCTTGATTCTT |
| | | CAACGCAAGGTCTATTAAGAATTTTACTGGGAAAAGCTGATGGCTAA |
| 127 | DP70 NADH- | ATGGCTGAAACTGACATTGCTATGCCAGAATCAACGCCTGTTGATTCA |
| | quinone | CGCCCAGCATTTGCAATTGTAGAAGAGCTCAAAGCCAAATTTGGTGA |
| | oxidoreductase | GAACTTCTATGTGCAAGCGACTTTTGAAGATTTTCCAACGGTCTGGGT |
| | subunit C/D | TGAGCGCGCGCGCGTACAAGATGTTTTAATGTTCTTGCGTAAAGTATC |
| | | ACGTCCATACGTGATGCTGTTCGACTTGTCTGCGGTAGATGAGCGTTT |
| | | ACGTACCCACCGTGACGGTTTACCTGCATCAGACTTCACTGTGTTTTA |
| | | TCATTTGTTGTCGCTAGAGCGCAACAGTGATATTCGTATTAAAGTTGC |
| | | GTTGAGTGAGAGTGATCTCAATCTTCCAACCGCAACCAACATTTGGCC |
| | | AAATGCCAACTGGTACGAACGTGAAGCTTACGATATGTTCGGGATCA |
| | | ATTTCGAAGGGCATCCAATGCTCCGTCGTATTTTGTTGCCAACCTATT |
| | | GGGAAGGTCACCCACTGCGTAAAGAATATTCTGCACGTGCGACTGAA |
| | | TATACACCGTATATGCAGAACCAAGCGAAGCAGGATTTCGAGCAAGA |
| | | ACATTTACGTTTTGTTCCTGAAGATTGGGGTCTATCACGCGGTAATGC |
| | | CGATGAAGATTTCATGTTCTTGAACTTAGGTCCAAACCATCCATCTGC |
| | | GCACGGTGCATTCCGTATCATTTTGCAGTTGGACGGTGAAGAAGTGA |
| | | AAGACTGTGTGCCTGATATTGGCTATCACCACCGTGGTGTGGAAAAG |
| | | ATGGCTGAACGTCAAACTTGGCATTCATTCATTCCATATACCGACCGT |
| | | GTTGACTACTTGGGTGGTTGTGCGCAAAACATGCCTTATGTGATGGGT |
| | | GTGGAGCAAATGGCAGGAATTACTGTTCCTGACCGTGCACAATGTAT |
| | | CCGTGTCATGATGTCTGAATTATTCCGTATCAATAACCATTTATTGTTT |
| | | ATTGGTACTGCAATTCAAGATGCCGGCGGTATGACGCCAGTCTTCTAT |
| | | ATGTTTGCCGATCGTCAAAAGATCTATGATGCGATTGAAGCGATTACA |
| | | GGCTACCGTATGCATCCAGCATGGTTCCGTATTGGCGGGACTGCGCAC |
| | | GACCTTCCAAACAATTGGCAACATCTGATTCGTGAAATTCTCGAATGG |
| | | ATGCCGAAGCGTATGAATGAATACTATACAGCTGCACTACGCAACTC |
| | | AGTATTTATTGGTCGTACCCGTAATGTTGCACAATACGATGCAAAATC |
| | | TGCATTGGCTTGGGGTGTAACAGGTACAGGTCTACGCGCGACAGGGA |
| | | TTGATTTCGACGTGCGTAAATACCGTCCGTATAGCGGTTATGAAAACT |
| | | ACGACTTCGACGTGCCTTTAGAATACGAAGGCGATGCTTACGCTCGTG |
| | | TGATGGTTCACTTCCGTGAAATTGAAGAATCACTGAAAATTGTGAAG |
| | | CAGTGCTTGGATAACATGCCATCTGGTCCATATAAAGCGGATCATCCT |
| | | TTGGCTGTTCCACCACCAAAAGACAAGACATTACAAGATATTGAAAC |
| | | TTTGATTACGCACTTCTTGAGCGTGTCATGGGGTCCTGTGATGCCTGC |
| | | GGGTGAAGCGTCTGTAATGGCTGAAGTGGTAAAAGGTGCATCGAACT |
| | | ACTACTTGACTTCAGACAAGTCAACCATGAGTTATCGTACCCGTATTC |
| | | GTACACCAACTTTCACGCACTTACAGCAAATGCCTTCTGTGATTAATG |
| | | GCAGTCTTGTATCTGACTTGATCATTTATTTAGCGACCATTGACGTCG |
| | | TAATGGCTGACGTGGATCGCTAG |
| 128 | DP70 Protein | ATGGATGATAATAAAAGTAAGGCGCTTAATGCTGCCCTAAGCCAGAT |
| | RecA | TGAAAAACAATTTGGTAAAAATACCGTAATGCGTCTTGGTGATAATA |
| | | CCGTATTGGCCGTTGAAGCGGTCTCTACAGGTTCTTTAACACTAGACA |
| | | TTGCACTTGGTATTGGTGGCTTACCAAAAGGTCGTATCGTTGAAATTT |
| | | ACGGTCCTGAATCTTCTGGTAAAACCACAATGACATTGCAAGCGATT |
| | | GCACAATGTCAAAAAGCCGGTGGTACTTGTGCTTTTATCGATGCAGA |
| | | ACATGCACTCGATCCTCAGTATGCACGTAAGCTTGGTGTCGACCTTGA |
| | | CAACCTGTTGGTTTCTCAACCAGACCACGGTGAACAAGCCCTTGAAAT |
| | | TGCAGACATGTTAGTCCGCTCTGGTGCTATTGACATGATCGTTGTCGA |
| | | TTCCGTGGCTGCACTGACACCTCGCGCTGAAATTGAAGGTGAAATGG |
| | | GCGACTCACATATGGGCTTACAAGCACGTTTGATGAGTCAGGCATTA |
| | | CGTAAAATTACTGGTAATGCAAAACGCTCAAACTGTATGGTGATCTTC |
| | | ATTAACCAAATCCGTATGAAGATTGGTGTAATGTTTGGTAGCCCTGAA |
| | | ACCACAACAGGTGGTAATGCACTCAAATTCTACGCTTCTGTACGTTTG |
| | | GATATCCGTCGTATTGGTCAAGTGAAAGAAGGCGATGAAATTGTCGG |
| | | TTCAGAAACCCGCGTTAAAGTCGTAAAAAATAAAATGGCACCTCCTT |
| | | TTAAGGAAGCGTTATTCCAAATTTTATATGGCAAAGGTGTCAATCAAC |
| | | TGGGTGAACTGGTTGATCTTGCTGTTGCGCAAGAACTGGTACAAAAA |
| | | GCAGGTGCTTGGTATTCATATCAAGGCAATAAAATTGGTCAAGGTAA |
| | | AAACAACGTGATCCGCCATTTAGAGGAAAATCCTCAAATTGCACAAG |
| | | AACTTGATCGCCTGATTCGTGAAAAATTGTTGACACCAACGACCACG |
| | | CCTATTGAAGAAAAAGATGAAGTAGAACCAGACTTTCTAGATGCTTA |
| | | A |
| 129 | DP70 RNA | ATGAGCGATATGACTTCCCCTACTTCGCAAGTAGCGGCTCTGATTAGC |
| | polymerase sigma | CGAGGCAAAGAGCAAGGTTACTTAACTTACGCTGAGGTTAACGATCA |
| | factor RpoD | TCTCCCAGACTCGATCACGGAAAGCGAACAGATTGAAGACATTATTC |
| | | AAATGCTTCAAGATGTCGGCATTCCAGTGCATGAACGTGCGCCTGAA |
| | | TCTGATGACACCATGTTCGACGGTAACAATGCAGAAGCAACCGATGA |
| | | AGTCGCTGAAGAAGAAGCGGCAGCTGTTCTTGCTTCAGTTGAAAGCG |
| | | AACCTGGTCGTACCACCGATCCAGTACGTATGTACATGCGTGAAATG |
| | | GGAACGGTTGAACTATTAACGCGTGAAGGCGAAATTAGCATTGCAAA |
| | | ACGCATTGAAGAAGGTATTCGTGACGTTCTTCATTCGATTGCGTACTG |
| | | GCCAAATGCAGTTGAAGTTGTATTAAAAGAATATAGCGATGTTGCTG |
| | | AAGGCGAACGTCGTCTTGCTGATATTTTATCTGGTTATTTAGACCCAG |
| | | AATCTGACGAAGAAATTCCAGAAGTTTTAGAAGAAGAAGCTGAAATT |
| | | GTTGAAGATGATGAAGCGACGACTAAAACCACTAAAGATGTAAAATT |
| | | GGACGATGACGAAGAAGAAGAATCTGAAAGTGATGATGATTCTGAA |
| | | GGTGAGTCTGGTCCAGATCCAGAAATTGCACGTGTTCGTTTCACTGAA |
| | | TTAGAAGATGCGTGGAAAGTAACCAAAGCCACCATTGAAAAGCATGG |
| | | CCGTAACAGCAAACAAGCAGATGAAGCGCTTGAAGCTCTTGCAACTG |
| | | TGTTTATGATGTTCAAATTTACACCACGTTTATTTGAAATCATTTCAGA |
| | | AATGATTCGTGGCACGCATGAACAAATTCGTACAGCAGAACGTGAAG |
| | | TGATGCGTTACGCAGTTCGTCGTGGTCGTATGGACCGTACCCAATTCC |
| | | GTACATCGTTCCCAGGCCAAGAGTCAAATCCAGCTTGGTTAGATGAA |
| | | CAAATTGCTAAAGCACCTGCGGATCAAAAAGGTTATTTAGAAAAAGT |
| | | ACGTCCAGATGTTGTTGCATTCCAGCAAAAGATTGCCGATATCGAAA |
| | | AAGAATTGGGCTTAGATGTTAAAGACATCAAAGACATTTCTAAACGT |
| | | ATGGCTGTGGGTGAAGCGAAAGCACGTCGCGCGAAAAAAGAAATGG |
| | | TTGAAGCAAACTTACGTTTGGTGATTTCGATTGCGAAAAAATATACCA |
| | | ACCGTGGTTTACAATTCCTTGACTTGATTCAAGAAGGTAACATCGGTT |
| | | TGATGAAAGCCGTAGACAAGTTTGAATACCGTCGTGGTTATAAATTCT |
| | | CGACTTATGCAACTTGGTGGATTCGTCAGGCGATTACCCGTTCGATTG |
| | | CCGATCAAGCACGTACCATCCGTATTCCAGTACACATGATCGAAACC |
| | | ATTAACAAGATCAACCGTGTATCTCGTCAACTTCTTCAAGAAATGGGC |
| | | CGTGAGCCTACCCCTGAAGAATTAGGCGAACGTCTGGAAATGGACGA |
| | | AGTTAAAGTACGTAAAGTGCTGAAAATTGCCAAAGAACCGATTTCGA |
| | | TGGAAACACCGATTGGTGATGACGAAGATTCGCATCTTGGTGACTTC |
| | | ATTGAAGATGGTAACATTACCTCTCCAATTGATGCCGCGACTTCTGAA |
| | | GGCTTAAAAGAAGCAACACGTGAAGTGCTGGAAAACTTGACCGAACG |
| | | TGAAGCGAAAGTCTTAAAAATGCGTTTTGGTATTGATATGCCAACCG |
| | | ACCATACTTTAGAAGAAGTGGGTAAACAATTTGATGTAACACGTGAA |
| | | CGTATTCGTCAGATTGAAGCCAAAGCTTTACGTAAATTACGTCACCCT |
| | | TCTCGTTCTGAACACTTACGTTCATTCCTAGAAAATGACTAA |
| 130 | DP71 Glutamine- | ATGAGTGAGGCTGAAGCCCGCCCAACAAATTTTATCCGTCAGATTATT |
| | IRNA ligase | GATGAAGATCTGGCGACCGGGAAACACAATACCGTTCACACCCGTTT |
| | | CCCGCCTGAGCCTAATGGCTATTTGCATATCGGCCATGCGAAGTCTAT |
| | | CTGCCTGAATTTCGGCATTGCGCAAGACTACCAGGGTCAGTGCAATCT |
| | | GCGTTTTGACGATACTAACCCGGCAAAAGAAGACATCGAATTCGTTG |
| | | AGTCGATCAAATACGACGTCCAGTGGCTGGGCTTCGACTGGAGCGGT |
| | | GATATTCACTACTCCTCAGACTATTTCGATCAACTGCACGCATACGCG |
| | | CTGGAGCTAATCAACAAAGGTCTGGCGTACGTTGACGAACTGTCTCC |
| | | CGATCAAATTCGCGAATACCGTGGTTCGCTGACCGCACCGGGCAAAA |
| | | ACAGCCCGTATCGCGATCGCAGCGTGGAAGAAAATATCGCGCTGTTT |
| | | GAAAAAATGCGTAACGGTGAATTCGCCGAAGGTGCCGCTTGCCTGCG |
| | | TGCCAAAATCGATATGGCGTCGCCATTCTTCGTGATGCGCGATCCGGT |
| | | CATCTACCGTATTAAGTTTGCCGAACATCATCAGACTGGCACAAAATG |
| | | GTGCATCTACCCGATGTACGATTTCACTCACTGCATTTCCGATGCGCT |
| | | GGAAGGGATCACCCATTCACTGTGTACGCTGGAATTCCAGGACAACC |
| | | GCCGTCTGTACGACTGGGTACTGGATAACATCACTATTCCATGCCATC |
| | | CGCGTCAGTATGAGTTCTCCCGTCTGAATCTTGAATACTCCATCATGT |
| | | CCAAGCGTAAGCTGAACCTGCTGGTGACGGATAAGATTGTAGAAGGT |
| | | TGGGACGATCCGCGTATGCCGACGGTTTCCGGTCTGCGTCGCCGTGGT |
| | | TATACCGCCGCGTCTATCCGCGAATTCTGCCGTCGTATCGGCGTGACC |
| | | AAGCAGGACAACAACGTTGAAATGATGGCGCTGGAATCCTGTATTCG |
| | | TGACGATCTGAACGAAAACGCACCGCGCGCCATGGCCGTTATTAACC |
| | | CGGTTAAAGTTGTCATTGAGAACTTCACCGGTGATGACGTGCAAATG |
| | | GTGAAAATGCCGAATCATCCGAGCAAACCGGAAATGGGCACCCGCGA |
| | | AGTGCCGTTCACCCGTGAGATTTACATCGATCAGGCTGATTTCCGCGA |
| | | AGAAGCGAACAAACAGTACAAACGTCTGGTGCTGGGCAAAGAAGTTC |
| | | GCCTGCGCAATGCGTATGTGATCAAAGCGGAACACATCGAGAAAGAC |
| | | GCGGAAGGGAATATCACCACCATCTTCTGTTCTTACGATATCGATACG |
| | | CTGAGCAAAGATCCCGCTGATGGCCGTAAGGTGAAAGGCGTGATTCA |
| | | CTGGGTTTCTGCTTCTGAAGGTAAACCGGCAGAATTTCGCCTGTATGA |
| | | CCGTCTGTTCAGTGTTGCGAACCCTGGCCAGGCTGAAGATTTCCTGAC |
| | | CACCATCAACCCGGAATCTCTGGTGATTGCTCAGGGCTTCGTTGAGCC |
| | | GTCTCTGGTCGCTGCTCAGGCAGAAGTCAGTGTGCAGTTCGAACGTG |
| | | AAGGTTACTTCTGTGCCGACAGCCGCTATTCAAGTGCTGAGCATCTGG |
| | | TGTTCAACCGCACCGTCGGCCTTCGCGACACCTGGGAAAGCAAACCC |
| | | GTCGCCTGA |
| 131 | DP71 DNA | ATGTCGAATTCTTATGACTCCTCAAGTATCAAGGTATTAAAAGGGCTG |
| | gyrase subunit B | GACGCGGTGCGTAAGCGCCCCGGCATGTATATCGGCGATACCGATGA |
| | | CGGCACTGGTCTGCACCACATGGTATTCGAGGTTGTGGACAACGCTAT |
| | | CGACGAAGCCCTCGCGGGCCACTGTAAAGAGATTCAGGTCACGATCC |
| | | ATGCGGATAACTCTGTTTCCGTACAGGATGATGGTCGTGGTATTCCTA |
| | | CCGGCATTCACGAAGAAGAGGGCGTTTCTGCTGCTCAGGTCATCATG |
| | | ACCGTACTTCATGCCGGCGGTAAATTTGACGATAACTCGTACAAAGTC |
| | | TCCGGCGGTCTGCATGGCGTGGGTGTTTCCGTCGTTAACGCCCTGTCG |
| | | GAAAAACTGGAGCTGGTTATCCGCCGTGAAGGCAAAGTGCACACCCA |
| | | GACTTACGTCCACGGTGAGCCGCAGGATCCGCTGAAAGTGGTTGGCG |
| | | ATACCGAGGCGACCGGTACGACCGTGCGCTTCTGGCCAAGCTACGCC |
| | | ACCTTCACCAATCAAACAGAATTCGAGTATGACATTCTGGCGAAACG |
| | | CCTCCGTGAGCTGTCATTCCTGAACTCTGGTGTGGCGATCCGCCTGCT |
| | | CGACAAACGCGATGGCAAGAACGATCACTTCCATTATGAAGGCGGTA |
| | | TCAAAGCTTTCGTGGAATACCTGAACAAAAACAAAACCCCAATCCAC |
| | | CCAACCGTGTTCTATTTCTCCACCGTGAAAGACGATATCGGTGTGGAA |
| | | GTGGCGTTGCAGTGGAATGATGGTTTCCAGGAAAATATTTACTGCTTT |
| | | ACCAACAATATCCCTCAGCGCGACGGCGGCACCCATCTGGTAGGCTT |
| | | CCGTTCTGCGATGACCCGTACGCTTAACGCGTATATGGATAAAGAAG |
| | | GCTACAGCAAGAAATCCAAAATCAGCGCCACCGGTGATGATGCCCGT |
| | | GAAGGCCTGATCGCCGTGGTTTCGGTAAAAGTGCCGGATCCTAAGTT |
| | | CTCCTCTCAGACCAAAGACAAACTGGTTTCTTCCGAAGTGAAGACCG |
| | | CCGTTGAGTCTCTGATGAACGAGAAGCTGGTTGATTATCTGATGGAA |
| | | AACCCGGCCGACGCGAAAATCGTTGTCGGTAAAATCATCGATGCAGC |
| | | CCGTGCGCGTGAAGCCGCGCGTAAAGCACGTGAAATGACCCGTCGTA |
| | | AAGGCGCGCTCGATCTGGCCGGTCTGCCAGGCAAACTGGCTGACTGT |
| | | CAGGAACGCGACCCGGCACATTCCGAACTGTACTTAGTGGAAGGGGA |
| | | CTCAGCGGGCGGCTCTGCAAAACAAGGCCGTAACCGTAAGAACCAGG |
| | | CGATTCTGCCGTTGAAAGGGAAAATCCTCAACGTTGAGAAAGCGCGC |
| | | TTCGACAAAATGCTCTCTTCTCAGGAAGTGGCGACGCTGATTACCGCG |
| | | CTCGGTTGCGGTATCGGCCGTGACGAATACAACCCGGATAAACTGCG |
| | | TTATCACAGCATCATCATCATGACCGATGCCGACGTCGATGGTTCGCA |
| | | CATCCGTACCCTGTTACTGACATTCTTCTACCGTCAGATGCCTGAAAT |
| | | TGTAGAGCGTGGCCACGTGTTTATCGCGCAGCCTCCGCTGTACAAAGT |
| | | GAAAAAAGGCAAACAGGAACAGTACATTAAAGATGATGAAGCGATG |
| | | GATCAGTATCAAATCTCTATCGCGATGGACGGGGCAACGTTACACGC |
| | | CAACGCCCATGCACCAGCACTGGCGGGCGAACCGCTGGAGAAACTGG |
| | | TGGCTGAACATCACAGCGTGCAGAAAATGATTGGCCGTATGGAACGT |
| | | CGTTATCCGCGTGCGCTGCTGAATAATCTGGTCTATCAGCCAACGCTG |
| | | GCGGGTGCTGAACTTGCCGACGAAGCGAAAGTGAAGGAATGGATTGA |
| | | AACGCTGGTGTCTCGTCTGAACGAGAAAGAGCAGCACGGCAGCAGCT |
| | | ACAGTGCGATCGTGCGCGAAAATCTTGAACACCAGCTGTTCGAGCCA |
| | | ATCCTGCGCATTCGTACTCACGGTGTGGATACCGACTACGATCTCGAT |
| | | GCAGACTTCATTCAGGGCGGCGAATACCGCAAAATCTGTACCCTGGG |
| | | TGAAAAACTGCGCGGCCTGATCGAAGAAGATGCTTACATCGAACGTG |
| | | GCGAACGCCGTCAGCCAGTGACCAGCTTCGAGCAGGCGCTGGAATGG |
| | | CTGGTGAAAGAGTCGCGTCGCGGTCTGTCGATTCAGCGTTATAAAGG |
| | | TCTGGGTGAAATGAACCCTGAGCAATTGTGGGAAACCACGATGGATC |
| | | CGACACAACGCCGCATGCTGCGCGTGACGGTGAAAGATGCTATCGCG |
| | | GCGGACCAGCTGTTCACCACGCTGATGGGCGATGCGGTTGAACCGCG |
| | | CCGCGCCTTCATCGAAGAGAACGCCCTTAAAGCTGCCAATATCGATA |
| | | TCTGA |
| 132 | DP71 Isoleucine | ATGAGTGACTACAAGAACACCCTGAATTTGCCGGAAACAGGGTTCCC |
| | tRNA ligase | GATGCGTGGCGATCTGGCCAAGCGTGAACCTGACATGCTGAAGAATT |
| | | GGTATGACCAGGATCTGTACGGGATTATTCGTGCTGCCAAGAAAGGC |
| | | AAGAAAACCTTTATCTTGCATGACGGCCCTCCGTATGCGAACGGCAG |
| | | CATTCATATTGGTCACTCAGTAAACAAAATTCTTAAAGACATGATCGT |
| | | TAAGTCCAAAGGACTGGCGGGCTTTGATGCGCCGTATGTTCCGGGCT |
| | | GGGATTGTCATGGTCTGCCGATTGAACTGAAAGTTGAACAGCTGATC |
| | | GGTAAGCCGGGCGAAAAAGTCACGGCGGCGGAATTCCGTGAAGCCTG |
| | | CCGCAAGTACGCTGCTGAACAGGTTGAAGGTCAGAAGAAAGACTTCA |
| | | TCCGTCTGGGCGTGCTCGGTGACTGGGATCATCCGTACCTGACCATGG |
| | | ACTTCAAAACAGAAGCCAACATCATTCGTGCCCTGGGTAAAATCATC |
| | | GGCAACGGTCACCTGCATAAAGGTGCGAAACCTGTTCACTGGTGTAC |
| | | CGATTGCGGATCTTCACTGGCTGAAGCCGAAGTCGAATATTACGACA |
| | | AAGTGTCTCCGTCTATCGACGTGACGTTTAATGCGACGGATGCCGCCG |
| | | CTGTTGCTGCGAAATTCGGTGCCACTGCTTTCAATGGCCCGGTTTCTC |
| | | TGGTCATCTGGACCACCACCCCGTGGACCATGCCAGCTAACCGCGCG |
| | | ATTTCACTCAACGCTGAGTTCTCTTATCAGCTGGTGCAGATTGAAGGT |
| | | CAGTGCCTGATCCTGGCTACCGATCTGGTAGAAAGCGTGATGAATCG |
| | | CGCCGGTATCGCTGAGTGGACTGTGCTGGGCGAATGTAAAGGTGCGG |
| | | ATCTTGAATTGCTTCGATTCCAGCATCCGTTCCTCGGTTTCGATGTTCC |
| | | GGCGATCCTCGGCGATCACGTTACTCTCGATGCCGGTACCGGTGCTGT |
| | | ACATACCGCACCTGGCCACGGTCCTGATGACTTTGTCATTGGCCAGAA |
| | | ATACGGTCTGGAAGTCGCAAACCCGGTTGGACCGAACGGCTGCTACC |
| | | TGCCGGGCACTTATCCGACGCTGGATGGCAAATTCGTCTTTAAAGCGA |
| | | ATGATCTGATCGTTGAATTGCTGCGTGAGAAGGGCGCACTGCTGCAC |
| | | GTTGAGAAAATGAACCACAGCTATCCGTGCTGCTGGCGTCACAAAAC |
| | | GCCGATCATCTTCCGCGCTACGCCACAATGGTTCATCAGCATGGATCA |
| | | GAAAGGTTTGCGTCAGAAGTCTCTGGAAGAGATCAAAGGCGTGCAGT |
| | | GGATCCCTGACTGGGGTCAGGCGCGTATCGAAAACATGGTCGCTAAC |
| | | CGTCCTGACTGGTGTATCTCCCGCCAGCGTACGTGGGGCGTACCGATG |
| | | TCTCTGTTCGTGCATAAAGATACCGAACAGCTTCATCCGCGCAGCCTT |
| | | GAGCTGATGGAAGAAGTGGCAAAACGCGTGGAAGCCGATGGCATTC |
| | | AGGCATGGTGGGATCTGAACCCTGAAGAGATTTTGGGTGCAGACGCT |
| | | GCCGATTACGTCAAAGTGCCGGATACGCTGGACGTCTGGTTTGACTCC |
| | | GGTTCCACGCACTCCTCCGTTGTGGATGTGCGCCCTGAGTTCAACGGT |
| | | CATTCACCGGATCTGTATCTGGAAGGTTCTGACCAGCATCGCGGCTGG |
| | | TTCATGTCTTCTCTGATGATTTCTACGGCGATGAAAGGCAAAGCGCCT |
| | | TACAAACAAGTACTGACTCACGGTTTCACCGTCGATGGTCAGGGCCG |
| | | TAAAATGTCTAAATCCATCGGTAACACCATCGCGCCTCAGGATGTGAT |
| | | GAATAAGCTGGGTGGCGACATCCTGCGTTTGTGGGTGGCATCTACGG |
| | | ATTACACCGGCGAAATCGCCGTGTCCGACGAAATCCTCAAACGTGCT |
| | | GCCGATTCTTATCGCCGTATCCGTAACACCGCGCGCTTCCTGCTGGCG |
| | | AACCTTAACGGTTTCGATCCGGCGCTGCACAGCGTGGCACCGGAAGA |
| | | GATGGTTGTGCTGGATCGCTGGGCGGTTGGCCGCGCGAAAGCTGCAC |
| | | AAGACGAGATCATTGCTGCGTACGAAGCCTATGATTTCCACGGCGTT |
| | | GTTCAGCGTCTGATGCAGTTCTGCTCGATCGAAATGGGTTCGTTCTAT |
| | | CTGGATATCATTAAAGATCGCCAGTACACCGCGAAGAGCGACAGCGT |
| | | TGCGCGCCGCAGCTGCCAGACCGCGCTGTATCACATCTGCGAAGCAC |
| | | TGGTTCGCTGGATGGCGCCAATCATGTCCTTCACTGCCGATGAAATCT |
| | | GGGCTGAACTGCCAGGTCATCGCGAGAAGTTCGTCTTTACTGAAGAA |
| | | TGGTACGACGGTCTGTTTGGCCTGATCGGTAACGAATCCATGAACGAT |
| | | GCGTTCTGGGATGAGCTGCTGAAAGTGCGTGGTGAAGTGAACAAAGT |
| | | GATCGAACAGGCGCGTGCTGATAAACGTCTGGGCGGTTCTCTGGAAG |
| | | CAGCCGTGACCTTATATGCAGACGACGCGCTGGCAACAGACCTGCGT |
| | | TCTCTGGGTAACGAACTGCGCTTTGTGCTCCTGACTTCCGGTGCGAAA |
| | | GTCGCCGCGCTGTCTGAAGCTGATGACTCAGCGCAGGCCAGCGAATT |
| | | GTTGAAAGGACTGAAAATTGGTCTGGCGAAAGCAGAAGGCGAGAAG |
| | | TGCCCGCGCTGCTGGCATTTCACCACTGATATCGGCCAGAATGCGGA |
| | | ACACAGTGACATCTGTGGCCGTTGTGTGACTAACATTGCCGGTGACG |
| | | GCGAAGAGCGTAAGTTTGCATAA |
| 133 | DP71 NADH- | ATGTCAGAACTTACTCATATTAATGCTTCCGGCGACGCCCACATGGTG |
| | quinone | GATGTCTCCGGTAAAGACGACACCGTTCGTGAAGCCCGTGCCGAAGC |
| | oxidoreductase | CTTTGTTGAAATGGCCGAAAGCACGCTGGCGATGATCATCGGCGGTA |
| | subunit C/D | ATCACCATAAGGGTGACGTGTTCGCGACCGCGCGGATTGCCGGTATT |
| | | CAGGCAGCGAAGAAAACCTGGGATCTGATCCCGCTGTGTCATCCGCT |
| | | GTTGCTGACCAAGGTGGAAGTGAATCTTGAAGCGCAGCCAGAATTTA |
| | | ATCGTGTACGTATTGAATCCCGCTGCCGCCTGAGCGGTAAAACCGGC |
| | | GTCGAGATGGAAGCGCTGACCTTCAAGCCTGAAGACTGGGGAATGAA |
| | | GCGCGGCACCGAAAACGAGGACTTCATGTTCCTCAACCTCGGACCTA |
| | | ACCATCCGTCTGCGCACGGTGCGTTCCGCATCATCCTGCAGCTTGATG |
| | | GCGAAGAAATTGTCGACTGTGTACCGGACGTCGGTTACCACCACCGT |
| | | GGTGCTGAGAAGATGGGCGAGCGCCAGTCATGGCACAGCTACATTCC |
| | | ATACACGGACCGTATCGAATACCTCGGCGGTTGCGTTAACGAGATGC |
| | | CATACGTACTGGCTGTTGAAAAACTGGCGGGTATCGTCGTGCCGGAT |
| | | CGCGTTAACACCATCCGCGTGATGCTGTCTGAACTGTTCCGTATCAAC |
| | | AGCCACCTGCTGTACATCTCTACGTTTATTCAGGACGTGGGCGCGATG |
| | | ACGCCAGTGTTCTTCGCCTTTACCGATCGTCAGAAAATTTACGATCTG |
| | | GTGGAAGCGATCACCGGTTTCCGTATGCACCCGGCCTGGTTCCGTATT |
| | | GGTGGCGTTGCACACGACCTGCCGAAAGGCTGGGAGCGTCTGCTGCG |
| | | TGAATTCCTTGACTGGATGCCAGCCCGTCTGGATTCCTACGTCAAGGC |
| | | AGCGCTGAAAAACACCATTCTGATTGGACGTTCCAAAGGCGTAGCAG |
| | | CATACAACGCCGATGATGCGCTGGCGTGGGGCACCACCGGTGCTGGC |
| | | CTGCGTGCGACCGGGATCGACTTCGATGTCCGCAAATGGCGTCCATAT |
| | | TCAGGTTACGAAAACTTCGATTTTGAAGTGCCGGTCGGCGATGGCGTC |
| | | AGTGATTGCTATTCCCGCGTGATGCTAAAAGTGGAAGAGCTTCGTCA |
| | | GAGCCTGCGCATTCTGGAACAGTGCTACAAAAACATGCCGGAAGGCC |
| | | CGTTCAAGGCGGATCACCCGCTGACCACGCCGCCACCGAAAGAGCGT |
| | | ACGCTGCAACACATCGAAACCCTGATCACTCACTTCCTGCAAGTGTCG |
| | | TGGGGTCCGATCATGCCTGCGCAAGAATCTTTCCAGATGGTTGAAGCC |
| | | ACCAAAGGGATCAACAGCTACTACCTGACCAGTGACGGCAGCACCAT |
| | | GAGCTACCGCACGCGCGTCCGTACGCCAAGCTTCCCGCATTTGCAGC |
| | | AGATCCCGTCCGTAATCCGTGGCAGCCTGGTATCCGACCTGATCGTGT |
| | | ATCTGGGCAGTATCGATTTTGTAATGTCAGATGTGGACCGCTAA |
| 134 | DP71 Protein | ATGGCTATTGATGAGAACAAGCAAAAAGCGTTAGCTGCAGCACTGGG |
| | RecA | CCAGATTGAAAAGCAATTCGGTAAAGGCTCCATCATGCGTCTGGGTG |
| | | AAGATCGCTCTATGGACGTGGAAACGATCTCTACCGGCTCTTTGTCTC |
| | | TGGATATCGCGTTAGGCGCCGGTGGTTTGCCGATGGGCCGTATCGTTG |
| | | AGATTTATGGCCCGGAATCCTCCGGTAAAACTACGCTGACCCTTCAGG |
| | | TTATTGCTGCCGCACAGCGCGAAGGCAAAACCTGTGCGTTCATCGAT |
| | | GCGGAACATGCACTTGACCCTATCTACGCGAAGAAATTGGGCGTAGA |
| | | TATCGACAACCTGTTGTGTTCTCAGCCGGATACCGGCGAACAGGCTCT |
| | | GGAAATCTGTGACGCGCTGACCCGTTCAGGCGCGGTCGACGTTATCA |
| | | TCGTCGACTCCGTTGCTGCACTGACGCCAAAAGCAGAAATCGAAGGC |
| | | GAAATCGGTGACTCTCACATGGGCCTTGCGGCACGTATGATGAGCCA |
| | | GGCAATGCGTAAGCTTGCCGGTAACCTGAAAAACGCCAACACCTTGC |
| | | TGATCTTCATCAACCAGATCCGTATGAAAATCGGTGTGATGTTCGGTA |
| | | ACCCGGAAACCACCACCGGTGGTAACGCCCTGAAATTCTACGCCTCT |
| | | GTGCGTCTGGATATCCGCCGCATCGGCGCTATCAAAGAAGGCGACGT |
| | | GGTGATCGGCAGTGAAACGCGCGTGAAAGTTGTGAAGAACAAAATCG |
| | | CTGCGCCTTTCAAACAGGCTGAATTCCAGATCCTATACGGCGAAGGC |
| | | ATCAACATTAACGGCGAGCTGATCGATTTGGGCGTTAAGCACAAACT |
| | | GGTCGAAAAAGCCGGTGCATGGTACAGCTACAACGGCGAGAAGATTG |
| | | GTCAGGGTAAATCTAACTCCTGCAACTATCTGAAAGAAAACCCGAAA |
| | | ATCGCTGCTGAACTGGATAAAAAACTGCGTGATATGTTGTTGAGTGG |
| | | CACTGGTGAACTGGCCGCTGCAACCACAGCAGAACTTGCAGACGACG |
| | | ATATGGAAACCAGCGAAGAGTTTTAA |
| 135 | DP71 RNA | GGTAAGGAGCAAGGCTATCTGACCTTTGCTGAGGTCAATGACCATCT |
| | polymerase sigma | GCCGGAAGATATCGTCGACTCCGACCAGATCGAAGACATCATCCAGA |
| | factor RpoD | TGATTAACGACATGGGCATCCAGGTTCTTGAAGAAGCGCCGGACGCC |
| | | GATGATTTGATGCTGGCCGAAAACCGCCCTGATACCGATGAAGATGC |
| | | TGCAGAAGCAGCGGCTCAGGTGCTTTCCAGCGTTGAATCTGAAATTG |
| | | GCCGTACCACCGACCCTGTGCGTATGTATATGCGCGAAATGGGTACC |
| | | GTTGAGCTCCTGACCCGTGAAGGCGAAATCGACATCGCCAAACGTAT |
| | | CGAAGACGGTATCAATCAGGTCCAGTGCTCCGTTGCTGAATATCCTGA |
| | | AGCTATCACCTATTTGTTAGAGCAATATGACCGTGTTGAAGCAGGCG |
| | | AAGCACGTCTGTCTGATTTGATCACCGGTTTTGTTGATCCGAACGCCG |
| | | AAGAAGAAATCGCGCCGACTGCGACTCACGTGGGTTCTGAACTGACC |
| | | ACTGAAGAGCAAAATGATACCGACGACGATGAAGAAGACGACGACG |
| | | ATGCTGAAGACGACAACAGCATCGACCCGGAACTGGCGCGTCAGAAG |
| | | TTCACCGATCTGCGTGAGCAACATGAAGCGACCCGTGCCGTCATCAA |
| | | GAAAAATGGCCGTAGCCACAAAAGCGCCGCAGAAGAAATTCTGAAG |
| | | CTGTCCGATGTGTTTAAACAGTTCCGTCTGGTACCAAAACAGTTCGAT |
| | | TTCCTGGTGAACAGCATGCGCTCCATGATGGATCGCGTCCGTACTCAG |
| | | GAACGTCTGATCATGAAAGTGTGCGTTGAACAGTGCAAAATGCCGAA |
| | | GAAAAACTTCGTCAATCTGTTCGCCGGTAACGAAACCAGCAGTACCT |
| | | GGTTTGATGCTGCTCTGGCAATGGGTAAACCATGGTCTGAGAAGCTG |
| | | AAAGAAGTGACCGAAGACGTGCAGCGCGGCCTGATGAAACTGCGCC |
| | | AAATCGAAGAAGAAACTGGCCTGACTATCGAACAGGTAAAAGACATT |
| | | AACCGTCGCATGTCGATCGGCGAAGCGAAAGCACGCCGCGCGAAGA |
| | | AAGAGATGGTTGAAGCGAACTTACGTCTGGTTATCTCTATCGCGAAG |
| | | AAATACACCAACCGTGGCTTGCAGTTCCTTGACCTGATTCAGGAAGGT |
| | | AACATCGGCCTGATGAAAGCCGTTGATAAGTTTGAATATCGCCGTGG |
| | | TTATAAGTTCTCTACTTATGCGACCTGGTGGATCCGTCAGGCTATCAC |
| | | CCGCTCCATCGCCGACCAGGCACGTACCATCCGTATTCCGGTGCATAT |
| | | GATTGAGACCATCAACAAACTCAACCGTATTTCGCGCCAGATGTTGC |
| | | AGGAGATGGGCCGTGAGCCGACGCCGGAAGAGCTGGCTGAACGCAT |
| | | GCTGATGCCGGAAGACAAGATCCGTAAAGTGCTGAAAATTGCTAAAG |
| | | AGCCAATCTCCATGGAAACGCCAATCGGCGACGATGAAGATTCGCAT |
| | | CTGGGTGATTTCATCGAGGATACTACCCTCGAGCTGCCGCTGGATTCT |
| | | GCGACCTCTGAAAGCCTGCGTTCTGCAACGCACGACGTTCTGGCTGGC |
| | | CTGACCGCACGTGAAGCGAAAGTTCTGCGTATGCGTTTCGGTATCGAT |
| | | ATGAACACTGACCACACTCTGGAAGAAGTGGGCAAACAGTTCGACGT |
| | | AACCCGTGAACGTATCCGTCAGATCGAAGCCAAAGCGTTGCGTAAAC |
| | | TACGCCACCCAAGCCGCTCCGAAGTGCTGCGCAGCTTCCTCGACGACT |
| | | AG |
| 136 | DP71 DNA- | ATGGACCAGAACAACCCGTTGTCTGAGATCACGCACAAACGTCGTAT |
| | directed RNA | CTCTGCACTGGGCCCGGGCGGTTTGACCCGTGAACGTGCTGGCTTTGA |
| | polymerase | AGTTCGAGACGTACACCCGACGCACTACGGTCGCGTATGTCCAATCG |
| | subunit beta | AAACGCCAGAAGGTCCAAACATCGGTCTGATCAACTCATTATCTGTCT |
| | | ATGCACAGACAAATGAGTATGGTTTCCTGGAAACCCCTTACCGCCGT |
| | | GTGCGTGAAGGTATGGTTACCGATGAAATTAACTACCTGTCTGCCATC |
| | | GAAGAAGGCAACTTTGTTATCGCTCAGGCGAACTCCAACCTGGATGA |
| | | CGAAGGCCACTTCCTGGAAGATTTAGTCACTTGTCGTAGCAAAGGCG |
| | | AATCAAGCCTGTTCAGCCGCGACCAGGTTGACTACATGGACGTTTCTA |
| | | CCCAGCAGATCGTATCCGTTGGTGCTTCACTGATTCCATTCCTGGAAC |
| | | ACGATGACGCCAACCGTGCATTGATGGGTGCGAACATGCAACGTCAG |
| | | GCAGTTCCTACTCTGCGTGCTGATAAGCCGCTGGTAGGTACTGGTATG |
| | | GAACGTGCTGTTGCGGTTGACTCCGGTGTTACTGCCGTTGCCAAACGT |
| | | GGTGGTACTGTTCAGTACGTAGATGCATCCCGTATCGTTATTCGTGTT |
| | | AACGAAGAAGAGATGAATCCAGGCGAAGCAGGTATCGACATTTATAA |
| | | CCTGACTAAGTACACCCGTTCTAACCAGAACACCTGCATCAACCAGA |
| | | TGCCGTGTGTGAATCTGGGCGAGCCAATCGAGCGCGGCGACGTGCTG |
| | | GCAGATGGTCCGTCAACAGATCTGGGCGAACTGGCACTGGGTCAGAA |
| | | CATGCGTGTCGCGTTCATGCCTTGGAACGGTTACAACTTCGAAGACTC |
| | | CATCTTGGTCTCCGAACGTGTTGTGCAGGAAGATCGCTTCACGACCAT |
| | | CCATATCCAGGAACTGGCATGTGTGTCCCGTGACACAAAGTTAGGGC |
| | | CTGAAGAGATCACTGCTGATATCCCTAACGTGGGTGAAGCTGCGCTCT |
| | | CCAAACTGGATGAGTCCGGTATTGTGTATATCGGTGCTGAAGTGACC |
| | | GGTGGTGACATTCTGGTCGGTAAAGTTACGCCTAAAGGCGAAACCCA |
| | | GCTGACTCCAGAAGAGAAACTGCTGCGTGCGATCTTCGGTGAGAAAG |
| | | CGTCTGACGTTAAAGATTCTTCTCTGCGTGTACCAAACGGCGTTTCCG |
| | | GTACGATTATTGACGTGCAAGTCTTTACCCGCGATGGCGTGGAAAAA |
| | | GATAAGCGTGCGTTAGAAATCGAAGAAATGCAGCTGAAACAGGCTAA |
| | | GAAAGACCTGACTGAAGAGCTGCAAATTCTGGAAGCTGGTCTGTTTG |
| | | CACGTATCCAGTCCGCGCTGGTTGCTGGCGGTGTTGAAGCCGATAAG |
| | | CTGGGCAAATTGCCACGCGATCGTTGGCTTGAACTGTCACTGACTGAC |
| | | GAAGACAAACAGAATCAGTTGGAACAGCTTGCTGAACAGTACGACGA |
| | | ACTGAAATCCGAGTTTGAGAAAAAACTCGAAGCTAAACGTCGTAAAA |
| | | TCACTCAGGGCGATGACCTAGCACCAGGTGTGCTGAAAATCGTTAAA |
| | | GTGTACCTGGCCGTTAAACGTCAGATCCAACCTGGTGACAAAATGGC |
| | | AGGCCGCCACGGTAACAAAGGTGTTATCTCCAAGATCAACCCGATCG |
| | | AAGATATGCCTTACGATGAAAACGGGACTCCTGTTGACATCGTACTG |
| | | AACCCGCTGGGCGTTCCATCACGTATGAACATTGGTCAGATTTTAGAA |
| | | ACCCACCTGGGTATGGCCGCGAAAGGTATTGGTGAAAAAATCAATGC |
| | | CATGCTTAAGAAACATGAAGAAGTTTCTAAGCTGCGCGAGTTCATCC |
| | | AGCGTGCCTATGATCTGGGCGACGACGTACGTCAGAAAGTTGATCTG |
| | | ACCACCTTCACCGATGATGAAGTATTGCGTTTGGCTGAAAACCTGAA |
| | | AAAGGGTATGCCAATTGCAACACCAGTCTTCGACGGTGCGAAAGAGA |
| | | CAGAGATCAAGCAACTGCTTGAAATGGGCGGCGTCCCAACCTCTGGC |
| | | CAGATCACACTGTTTGACGGCCGTACCGGCGAGCAATTCGAGCGCCA |
| | | GGTTACCGTCGGCTACATGTACATGCTGAAACTGAACCACCTGGTTGA |
| | | CGATAAGATGCATGCGCGTTCTACCGGTTCTTACAGCCTTGTTACTCA |
| | | GCAGCCGCTGGGTGGTAAAGCTCAGTTCGGTGGTCAGCGCTTCGGTG |
| | | AGATGGAAGTGTGGGCACTGGAAGCATACGGTGCCGCTTATACCCTG |
| | | CAGGAAATGCTGACTGTTAAGTCCGATGACGTGAACGGCCGTACTAA |
| | | GATGTATAAAAACATCGTAGATGGCGATCACCGGATGGAACCAGGCA |
| | | TGCCGGAATCATTCAACGTACTGTTGAAAGAAATCCGCTCTCTGGGTA |
| | | TCAACATCGAGCTGGAAGACGAGTAA |
| 137 | DP72 16S rRNA | TTCGGAGAGTTTGATCCTGGCTCAGGACGAACGCTGGCGGCGTGCCT |
| | | AATACATGCAAGTCGAGCGGACAGAAGGGAGCTTGCTCCCGGATGTT |
| | | AGCGGCGGACGGGTGAGTAACACGTGGGTAACCTGCCTGTAAGACTG |
| | | GGATAACTCCGGGAAACCGGAGCTAATACCGGATAGTTCCTTGAACC |
| | | GCATGGTTCAAGGATGAAAGACGGTTTCGGCTGTCACTTACAGATGG |
| | | ACCCGCGGCGCATTAGCTAGTTGGTGGGGTAATGGCTCACCAAGGCG |
| | | ACGATGCGTAGCCGACCTGAGAGGGTGATCGGCCACACTGGGACTGA |
| | | GACACGGCCCAGACTCCTACGGGAGGCAGCAGTAGGGAATCTTCCGC |
| | | AATGGACGAAAGTCTGACGGAGCAACGCCGCGTGAGTGATGAAGGTT |
| | | TTCGGATCGTAAAGCTCTGTTGTTAGGGAAGAACAAGTGCGAGAGTA |
| | | ACTGCTCGCACCTTGACGGTACCTAACCAGAAAGCCACGGCTAACTA |
| | | CGTGCCAGCAGCCGCGGTAATACGTAGGTGGCAAGCGTTGTCCGGAA |
| | | TTATTGGGCGTAAAGGGCTCGCAGGCGGTTTCTTAAGTCTGATGTGAA |
| | | AGCCCCCGGCTCAACCGGGGAGGGTCATTGGAAACTGGGAAACTTGA |
| | | GTGCAGAAGAGGAGAGTGGAATTCCACGTGTAGCGGTGAAATGCGTA |
| | | GAGATGTGGAGGAACACCAGTGGCGAAGGCGACTCTCTGGTCTGTAA |
| | | CTGACGCTGAGGAGCGAAAGCGTGGGGAGCGAACAGGATTAGATAC |
| | | CCTGGTAGTCCACGCCGTAAACGATGAGTGCTAAGTGTTAGGGGGTT |
| | | TCCGCCCCTTAGTGCTGCAGCTAACGCATTAAGCACTCCGCCTGGGGA |
| | | GTACGGTCGCAAGACTGAAACTCAAAGGAATTGACGGGGGCCCGCAC |
| | | AAGCGGTGGAGCATGTGGTTTAATTCGAAGCAACGCGAAGAACCTTA |
| | | CCAGGTCTTGACATCCTCTGACAACCCTAGAGATAGGGCTTTCCCTTC |
| | | GGGGACAGAGTGACAGGTGGTGCATGGTTGTCGTCAGCTCGTGTCGT |
| | | GAGATGTTGGGTTAAGTCCCGCAACGAGCGCAACCCTTGATCTTAGTT |
| | | GCCAGCATTTAGTTGGGCACTCTAAGGTGACTGCCGGTGACAAACCG |
| | | GAGGAAGGTGGGGATGACGTCAAATCATCATGCCCCTTATGACCTGG |
| | | GCTACACACGTGCTACAATGGACAGAACAAAGGGCTGCGAGACCGCA |
| | | AGGTTTAGCCAATCCCATAAATCTGTTCTCAGTTCGGATCGCAGTCTG |
| | | CAACTCGACTGCGTGAAGCTGGAATCGCTAGTAATCGCGGATCAGCA |
| | | TGCCGCGGTGAATACGTTCCCGGGCCTTGTACACACCGCCCGTCACAC |
| | | CACGAGAGTTTGCAACACCCGAAGTCGGTGAGGTAACCTTTATGGAG |
| | | CCAGCCGCCGAAGGTGGGGCAGATGATTGGGGTGAAGTCGTAACAAG |
| | | GTAGCCGTATCGGAAGGTGCGGCTGGATCACCTCCTTT |
| 138 | DP73 16S rRNA | AACGGAGAGTTTGATCCTGGCTCAGGACGAACGCTGGCGGCGTGCCT |
| | | AATACATGCAAGTCGAGCGGACAGAAGGGAGCTTGCTCCCGGACGTT |
| | | AGCGGCGGACGGGTGAGTAACACGTGGGCAACCTGCCCCTTAGACTG |
| | | GGATAACTCCGGGAAACCGGAGCTAATACCGGATAATCCCTTTCTCC |
| | | ACCTGGAGAGAGGGTGAAAGATGGCTTCGGCTATCACTAAGGGATGG |
| | | GCCCGCGGCGCATTAGCTAGTTGGTAAGGTAACGGCTTACCAAGGCG |
| | | ACGATGCGTAGCCGACCTGAGAGGGTGATCGGCCACACTGGGACTGA |
| | | GACACGGCCCAGACTCCTACGGGAGGCAGCAGTAGGGAATCTTCCGC |
| | | AATGGACGAAAGTCTGACGGAGCAACGCCGCGTGAGTGAGGAAGGC |
| | | CTTCGGGTCGTAAAGCTCTGTTGTGAGGGAAGAAGCGGTGCCGTTCG |
| | | AATAGGGCGGTACCTTGACGGTACCTCACCAGAAAGCCACGGCTAAC |
| | | TACGTGCCAGCAGCCGCGGTAATACGTAGGTGGCAAGCGTTGTCCGG |
| | | AATTATTGGGCGTAAAGCGCGCGCAGGCGGCTTCTTAAGTCTGATGT |
| | | GAAATCTCGGGGCTCAACCCCGAGCGGCCATTGGAAACTGGGGAGCT |
| | | TGAGTGCAGAAGAGGAGAGTGGAATTCCACGTGTAGCGGTGAAATGC |
| | | GTAGAGATGTGGAGGAACACCAGTGGCGAAGGCGACTCTCTGGTCTG |
| | | TAACTGACGCTGAGGCGCGAAAGCGTGGGGAGCAAACAGGATTAGA |
| | | TACCCTGGTAGTCCACGCCGTAAACGATGAGTGCTAGGTGTTAG |
| 139 | DP74 16S rRNA | GCCTAATACATGCAAGTCGTGCGGACCTTTTAAAAGCTTGCTTTTAAA |
| | | AGGTTAGCGGCGAACGGGTGAGTAACACGTGGGCAACCTGCCTGTAA |
| | | GATCGGGATAATGCCGGGAAACCGGGGCTAATACCGGATAGTTTTTT |
| | | CCTCCGCATGGAGGAAAAAGGAAAGACGGCTTCGGCTGTCACTTACA |
| | | GATGGGCCCGCGGCGCATTAGCTTGTTGGTGGGGTAACGGCTCACCA |
| | | AGGCAACGATGCGTAGCCGACCTGAGAGGGTGATCGGCCACATTGGG |
| | | ACTGAGACACGGCCCAAACTCCTACGGGAGGCAGCAGTAGGGAATCT |
| | | TCCGCAATGGACGAAAGTCTGACGGAGCAACGCCGCGTGAGTGAAGA |
| | | AGGCCTTCGGGTCGTAAAACTCTGTTGCCGGGGAAGAACAAGTGCCG |
| | | TTCGAACAGGGCGGCGCCTTGACGGTACCCGGCCAGAAAGCCACGGC |
| | | TAACTACGTGCCAGCAGCCGCGGTAATACGTAGGTGGCAAGCGTTGT |
| | | CCGGAATTATTGGGCGTAAAGCGCGCGCAGGCGGCTTCTTAAGTCTG |
| | | ATGTGAAATCTTGCGGCTCAACCGCAAGCGGTCATTGGAAACTGGGA |
| | | GGCTTGAGTGCAGAAGAGGAGAGTGGAATTCCACGTGTAGCGGTGAA |
| | | ATGCGTAGAGATGTGGAGGAACACCAGTGGCGAAGGCGGCTCTCTGG |
| | | TCTGTAACTGACGCTGAGGCGCGAAAGCGTGGGGAGCAAACAGGATT |
| | | AGATACCCTGGTAGTCCACGCCGTAAACGATGAGTGCTAAGTGTTAG |
| | | AGGGTTTCCGCCCTTTAGTGCTGCAGCTAACGCATTAAGCACTCCGCC |
| | | TGGGGAGTACGGCCGCAAGGCTGAAACTCAAAGGAATTGACGGGGG |
| | | CCCGCACAAGCGGTGGAGCATGTGGTTTAATTCGAAGCAACGCGAAG |
| | | AACCTTACCAGGTCTTGACATCCTCTGACCTCCCTGGAGACAGGGCCT |
| | | TCCCCTTCGGGGGACAGAGTGACAGGTGGTGCATGGTTGTCGTCAGC |
| | | TCGTGTCGTGAGATGTTGGGTTAAGTCCCGCAACGAGCGCAACCCTTG |
| | | ACCTTAGTTGCCAGCATTCAG |
| 140 | DP75 16S rRNA | TGAAGAGTTTGATCATGGCTCAGATTGAACGCTGGCGGCAGGCCTAA |
| | | CACATGCAAGTCGAGCGGTAGAGAGAAGCTTGCTTCTCTTGAGAGCG |
| | | GCGGACGGGTGAGTAATGCCTAGGAATCTGCCTGGTAGTGGGGGATA |
| | | ACGTTCGGAAACGGACGCTAATACCGCATACGTCCTACGGGAGAAAG |
| | | CAGGGGACCTTCGGGCCTTGCGCTATCAGATGAGCCTAGGTCGGATT |
| | | AGCTAGTTGGTGAGGTAATGGCTCACCAAGGCGACGATCCGTAACTG |
| | | GTCTGAGAGGATGATCAGTCACACTGGAACTGAGACACGGTCCAGAC |
| | | TCCTACGGGAGGCAGCAGTGGGGAATATTGGACAATGGGCGAAAGCC |
| | | TGATCCAGCCATGCCGCGTGTGTGAAGAAGGTCTTCGGATTGTAAAG |
| | | CACTTTAAGTTGGGAGGAAGGGTTGTAGATTAATACTCTGCAATTTTG |
| | | ACGTTACCGACAGAATAAGCACCGGCTAACTCTGTGCCAGCAGCCGC |
| | | GGTAATACAGAGGGTGCAAGCGTTAATCGGAATTACTGGGCGTAAAG |
| | | CGCGCGTAGGTGGTTCGTTAAGTTGGATGTGAAAGCCCCGGGCTCAA |
| | | CCTGGGAACTGCATTCAAAACTGACGAGCTAGAGTATGGTAGAGGGT |
| | | GGTGGAATTTCCTGTGTAGCGGTGAAATGCGTAGATATAGGAAGGAA |
| | | CACCAGTGGCGAAGGCGACCACCTGGACTGATACTGACACTGAGGTG |
| | | CGAAAGCGTGGGGAGCAAACAGGATTAGATACCCTGGTAGTCCACGC |
| | | CGTAAACGATGTCAACTAGCCGTTGGAATCCTTGAGATTTTAGTGGCG |
| | | CAGCTAACGCATTAAGTTGACCGCCTGGGGAGTACGGCCGCAAGGTT |
| | | AAAACTCAAATGAATTGACGGGGGCCCGCACAAGCGGTGGAGCATGT |
| | | GGTTTAATTCGAAGCAACGCGAAGAACCTTACCAGGCCTTGACATCC |
| | | AATGAACTTTCCAGAGATGGATGGGTGCCTTCGGGAACATTGAGACA |
| | | GGTGCTGCATGGCTGTCGTCAGCTCGTGTCGTGAGATGTTGGGTTAAG |
| | | TCCCGTAACGAGCGCAACCCTTGTCCTTAGTTACCAGCACGTTATGGT |
| | | GGGCACTCTAAGGAGACTGCCGGTGACAAACCGGAGGAAGGTGGGG |
| | | ATGACGTCAAGTCATCATGGCCCTTACGGCCTGGGCTACACACGTGCT |
| | | ACAATGGTCGGTACAAAGGGTTGCCAAGCCGCGAGGTGGAGCTAATC |
| | | CCATAAAACCGATCGTAGTCCGGATCGCAGTCTGCAACTCGACTGCG |
| | | TGAAGTCGGAATCGCTAGTAATCGCGAATCAGAATGTCGCGGTGAAT |
| | | ACGTTCCCGGGCCTTGTACACACCGCCCGTCACACCATGGGAGTGGG |
| | | TTGCACCAGAACGGGAGGACGGTTACCACGGTGTGATTCATGACTGG |
| | | GGTGAAGTCGTAACAAGGTAGCCGTAGGGGAACCTGCGGCTGGATCA |
| | | CCTCCTT |
| 141 | DP76 16S rRNA | CTTGAGAGTTTGATCCTGGCTCAGAACGAACGCTGGCGGCAGGCTTA |
| | | ACACATGCAAGTCGAGCGCCCCGCAAGGGGAGCGGCAGACGGGTGA |
| | | GTAACGCGTGGGAATCTACCTTTTGCTACGGAACAACAGTTGGAAAC |
| | | GACTGCTAATACCGTATGTGCCCTTCGGGGGAAAGATTTATCGGCAA |
| | | AGGATGAGCCCGCGTTGGATTAGCTAGTTGGTGAGGTAAAGGCTCAC |
| | | CAAGGCGACGATCCATAGCTGGTCTGAGAGGATGATCAGCCACACTG |
| | | GGACTGAGACACGGCCCAGACTCCTACGGGAGGCAGCAGTGGGGAA |
| | | TATTGGACAATGGGCGCAAGCCTGATCCAGCCATGCCGCGTGAGTGA |
| | | TGAAGGCCCTAGGGTTGTAAAGCTCTTTCACCGGTGAAGATAATGAC |
| | | GGTAACCGGAGAAGAAGCCCCGGCTAACTTCGTGCCAGCAGCCGCGG |
| | | TAATACGAAGGGGGCTAGCGTTGTTCGGATTTACTGGGCGTAAAGCG |
| | | CACGTAGGCGGATTTTTAAGTCAGGGGTGAAATCCCGGGGCTCAACC |
| | | CCGGAACTGCCTTTGATACTGGAAGTCTTGAGTATGGTAGAGGTGAG |
| | | TGGAATTCCGAGTGTAGAGGTGAAATTCGTAGATATTCGGAGGAACA |
| | | CCAGTGGCGAAGGCGGCTCACTGGACCATTACTGACGCTGAGGTGCG |
| | | AAAGCGTGGGGAGCAAACAGGATTAGATACCCTGGTAGTCCACGCCG |
| | | TAAACGATGAATGTTAGCCGTCGGGGGGTTTACCTTTCGGTGGCGCA |
| | | GCTAACGCATTAAACATTCCGCCTGGGGAGTACGGTCGCAAGATTAA |
| | | AACTCAAAGGAATTGACGGGGGCCCGCACAAGCGGTGGAGCATGTG |
| | | GTTTAATTCGAAGCAACGCGCAGAACCTTACCAGCCCTTGACATACC |
| | | GGTCGCGGACACAGAGATGTGTCTTTCAGTTCGGCTGGACCGGATAC |
| | | AGGTGCTGCATGGCTGTCGTCAGCTCGTGTCGTGAGATGTTGGGTTAA |
| | | GTCCCGCAACGAGCGCAACCCTCGCCTTTAGTTGCCAGCATTTAGTTG |
| | | GGCACTCTAAAGGGACTGCCAGTGATAAGCTGGAGGAAGGTGGGGAT |
| | | GACGTCAAGTCCTCATGGCCCTTACGGGCTGGGCTACACACGTGCTAC |
| | | AATGGTGGTGACAGTGGGCAGCAAGCACGCGAGTGTGAGCTAATCTC |
| | | CAAAAGCCATCTCAGTTCGGATTGCACTCTGCAACTCGAGTGCATGA |
| | | AGTTGGAATCGCTAGTAATCGCGGATCAGCATGCCGCGGTGAATACG |
| | | TTCCCGGGCCTTGTACACACCGCCCGTCACACCATGGGAGTTGGTTTT |
| | | ACCCGAAGGCACTGTGCTAACCGCAAGGAGGCAGGTGACCACGGTAG |
| | | GGTCAGCGACTGGGGTGAAGTCGTAACAAGGTAGCCGTAGGGGAACC |
| | | TGCGGCTGGATCACCTCCTTT |
| 142 | DP77 16S rRNA | TCGGAGAGTTTGATCCTGGCTCAGGATGAACGCTGGCGGCGTGCCTA |
| | | ATACATGCAAGTCGAGCGAACTGATTAGAAGCTTGCTTCTATGACGTT |
| | | AGCGGCGGACGGGTGAGTAACACGTGGGCAACCTGCCTGTAAGACTG |
| | | GGATAACTTCGGGAAACCGAAGCTAATACCGGATAGGATCTTCTCCT |
| | | TCATGGGAGATGATTGAAAGATGGTTTCGGCTATCACTTACAGATGG |
| | | GCCCGCGGTGCATTAGCTAGTTGGTGAGGTAACGGCTCACCAAGGCA |
| | | ACGATGCATAGCCGACCTGAGAGGGTGATCGGCCACACTGGGACTGA |
| | | GACACGGCCCAGACTCCTACGGGAGGCAGCAGTAGGGAATCTTCCGC |
| | | AATGGACGAAAGTCTGACGGAGCAACGCCGCGTGAGTGATGAAGGCT |
| | | TTCGGGTCGTAAAACTCTGTTGTTAGGGAAGAACAAGTACAAGAGTA |
| | | ACTGCTTGTACCTTGACGGTACCTAACCAGAAAGCCACGGCTAACTA |
| | | CGTGCCAGCAGCCGCGGTAATACGTAGGTGGCAAGCGTTATCCGGAA |
| | | TTATTGGGCGTAAAGCGCGCGCAGGCGGTTTCTTAAGTCTGATGTGAA |
| | | AGCCCACGGCTCAACCGTGGAGGGTCATTGGAAACTGGGGAACTTGA |
| | | GTGCAGAAGAGAAAAGCGGAATTCCACGTGTAGCGGTGAAATGCGTA |
| | | GAGATGTGGAGGAACACCAGTGGCGAAGGCGGCTTTTTGGTCTGTAA |
| | | CTGACGCTGAGGCGCGAAAGCGTGGGGAGCAAACAGGATTAGATAC |
| | | CCTGGTAGTCCACGCCGTAAACGATGAGTGCTAAGTGTTAGAGGGTT |
| | | TCCGCCCTTTAGTGCTGCAGCTAACGCATTAAGCACTCCGCCTGGGGA |
| | | GTACGGTCGCAAGACTGAAACTCAAAGGAATTGACGGGGGCCCGCAC |
| | | AAGCGGTGGAGCATGTGGTTTAATTCGAAGCAACGCGAAGAACCTTA |
| | | CCAGGTCTTGACATCCTCTGACAACTCTAGAGATAGAGCGTTCCCCTT |
| | | CGGGGGACAGAGTGACAGGTGGTGCATGGTTGTCGTCAGCTCGTGTC |
| | | GTGAGATGTTGGGTTAAGTCCCGCAACGAGCGCAACCCTTGATCTTA |
| | | GTTGCCAGCATTCAGTTGGGCACTCTAAGGTGACTGCCGGTGACAAA |
| | | CCGGAGGAAGGTGGGGATGACGTCAAATCATCATGCCCCTTATGACC |
| | | TGGGCTACACACGTGCTACAATGGATGGTACAAAGGGCTGCAAGACC |
| | | GCGAGGTCAAGCCAATCCCATAAAACCATTCTCAGTTCGGATTGTAG |
| | | GCTGCAACTCGCCTACATGAAGCTGGAATCGCTAGTAATCGCGGATC |
| | | AGCATGCCGCGGTGAATACGTTCCCGGGCCTTGTACACACCGCCCGTC |
| | | ACACCACGAGAGTTTGTAACACCCGAAGTCGGTGGAGTAACCGTAAG |
| | | GAGCTAGCCGCCTAAGGTGGGACAGATGATTGGGGTGAAGTCGTAAC |
| | | AAGGTAGCCGTATCGGAAGGTGCGGCTGGATCACCTCCTTT |
| 143 | DP78 16S rRNA | TTGAAGAGTTTGATCATGGCTCAGATTGAACGCTGGCGGCAGGCCTA |
| | | ACACATGCAAGTCGGACGGTAGCACAGAGAGCTTGCTCTTGGGTGAC |
| | | GAGTGGCGGACGGGTGAGTAATGTCTGGGGATCTGCCCGATAGAGGG |
| | | GGATAACCACTGGAAACGGTGGCTAATACCGCATAACGTCGCAAGAC |
| | | CAAAGAGGGGGACCTTCGGGCCTCTCACTATCGGATGAACCCAGATG |
| | | GGATTAGCTAGTAGGCGGGGTAATGGCCCACCTAGGCGACGATCCCT |
| | | AGCTGGTCTGAGAGGATGACCAGCCACACTGGAACTGAGACACGGTC |
| | | CAGACTCCTACGGGAGGCAGCAGTGGGGAATATTGCACAATGGGCGC |
| | | AAGCCTGATGCAGCCATGCCGCGTGTATGAAGAAGGCCTTCGGGTTG |
| | | TAAAGTACTTTCAGCGGGGAGGAAGGCGACGGGGTTAATAACCCTGT |
| | | CGATTGACGTTACCCGCAGAAGAAGCACCGGCTAACTCCGTGCCAGC |
| | | AGCCGCGGTAATACGGAGGGTGCAAGCGTTAATCGGAATTACTGGGC |
| | | GTAAAGCGCACGCAGGCGGTCTGTTAAGTCAGATGTGAAATCCCCGG |
| | | GCTTAACCTGGGAACTGCATTTGAAACTGGCAGGCTTGAGTCTTGTAG |
| | | AGGGGGGTAGAATTCCAGGTGTAGCGGTGAAATGCGTAGAGATCTGG |
| | | AGGAATACCGGTGGCGAAGGCGGCCCCCTGGACAAAGACTGACGCTC |
| | | AGGTGCGAAAGCGTGGGGAGCAAACAGGATTAGATACCCTGGTAGTC |
| | | CACGCCGTAAACGATGTCGACTTGGAGGTTGTTCCCTTGAGGAGTGG |
| | | CTTCCGGAGCTAACGCGTTAAGTCGACCGCCTGGGGAGTACGGCCGC |
| | | AAGGTTAAAACTCAAATGAATTGACGGGGGCCCGCACAAGCGGTGGA |
| | | GCATGTGGTTTAATTCGATGCAACGCGAAGAACCTTACCTACTCTTGA |
| | | CATCCAGCGAACTTAGCAGAGATGCTTTGGTGCCTTCGGGAACGCTG |
| | | AGACAGGTGCTGCATGGCTGTCGTCAGCTCGTGTTGTGAAATGTTGGG |
| | | TTAAGTCCCGCAACGAGCGCAACCCTTATCCTTTGTTGCCAGCGATTC |
| | | GGTCGGGAACTCAAAGGAGACTGCCGGTGATAAACCGGAGGAAGGT |
| | | GGGGATGACGTCAAGTCATCATGGCCCTTACGAGTAGGGCTACACAC |
| | | GTGCTACAATGGCGCATACAAAGAGAAGCGACCTCGCGAGAGCAAG |
| | | CGGACCTCACAAAGTGCGTCGTAGTCCGGATCGGAGTCTGCAACTCG |
| | | ACTCCGTGAAGTCGGAATCGCTAGTAATCGTGGATCAGAATGCCACG |
| | | GTGAATACGTTCCCGGGCCTTGTACACACCGCCCGTCACACCATGGG |
| | | AGTGGGTTGCAAAAGAAGTAGGTAGCTTAACCTTCGGGAGGGCGCTT |
| | | ACCACTTTGTGATTCATGACTGGGGTGAAGTCGTAACAAGGTAACCG |
| | | TAGGGGAACCTGCGGTTGGATCACCTCCTT |
| 144 | DP79 16S rRNA | TGAAGAGTTTGATCATGGCTCAGATTGAACGCTGGCGGCAGGCCTAA |
| | | CACATGCAAGTCGAGCGGTAGAGAGAAGCTTGCTTCTCTTGAGAGCG |
| | | GCGGACGGGTGAGTAATACCTAGGAATCTGCCTGATAGTGGGGGATA |
| | | ACGTTCGGAAACGGACGCTAATACCGCATACGTCCTACGGGAGAAAG |
| | | CAGGGGACCTTCGGGCCTTGCGCTATCAGATGAGCCTAGGTCGGATT |
| | | AGCTAGTTGGTGAGGTAATGGCTCACCAAGGCTACGATCCGTAACTG |
| | | GTCTGAGAGGATGATCAGTCACACTGGAACTGAGACACGGTCCAGAC |
| | | TCCTACGGGAGGCAGCAGTGGGGAATATTGGACAATGGGCGAAAGCC |
| | | TGATCCAGCCATGCCGCGTGTGTGAAGAAGGTCTTCGGATTGTAAAG |
| | | CACTTTAAGTTGGGAGGAAGGGCAGTTACCTAATACGTGACTGTCTTG |
| | | ACGTTACCGACAGAATAAGCACCGGCTAACTCTGTGCCAGCAGCCGC |
| | | GGTAATACAGAGGGTGCAAGCGTTAATCGGAATTACTGGGCGTAAAG |
| | | CGCGCGTAGGTGGTTTGTTAAGTTGAATGTGAAATCCCCGGGCTCAAC |
| | | CTGGGAACTGCATCCAAAACTGGCAAGCTAGAGTATGGTAGAGGGTA |
| | | GTGGAATTTCCTGTGTAGCGGTGAAATGCGTAGATATAGGAAGGAAC |
| | | ACCAGTGGCGAAGGCGACTACCTGGACTGATACTGACACTGAGGTGC |
| | | GAAAGCGTGGGGAGCAAACAGGATTAGATACCCTGGTAGTCCACGCC |
| | | GTAAACGATGTCAACTAGCCGTTGGGAGTCTTGAACTCTTAGTGGCGC |
| | | AGCTAACGCATTAAGTTGACCGCCTGGGGAGTACGGCCGCAAGGTTA |
| | | AAACTCAAATGAATTGACGGGGGCCCGCACAAGCGGTGGAGCATGTG |
| | | GTTTAATTCGAAGCAACGCGAAGAACCTTACCAGGCCTTGACATCCA |
| | | ATGAACTTTCTAGAGATAGATTGGTGCCTTCGGGAACATTGAGACAG |
| | | GTGCTGCATGGCTGTCGTCAGCTCGTGTCGTGAGATGTTGGGTTAAGT |
| | | CCCGTAACGAGCGCAACCCTTGTCCTTAGTTACCAGCACGTAATGGTG |
| | | GGCACTCTAAGGAGACTGCCGGTGACAAACCGGAGGAAGGTGGGGA |
| | | TGACGTCAAGTCATCATGGCCCTTACGGCCTGGGCTACACACGTGCTA |
| | | CAATGGTCGGTACAAAGGGTTGCCAAGCCGCGAGGTGGAGCTAATCC |
| | | CATAAAACCGATCGTAGTCCGGATCGCAGTCTGCAACTCGACTGCGT |
| | | GAAGTCGGAATCGCTAGTAATCGTGAATCAGAATGTCACGGTGAATA |
| | | CGTTCCCGGGCCTTGTACACACCGCCCGTCACACCATGGGAGTGGGTT |
| | | GCACCAGAAGTAGCTAGTCTAACCTTCGGGAGGACGGTTACCACGGT |
| | | GTGATTCATGACTGGGGTGAAGTCGTAACAAGGTAGCCGTAGGGGAA |
| | | CCTGCGGCTGGATCACCTCCTT |
| 145 | DP80 16S rRNA | CTTGAGAGTTTGATCCTGGCTCAGAGCGAACGCTGGCGGCAGGCTTA |
| | | ACACATGCAAGTCGAGCGGGCACCTTCGGGTGTCAGCGGCAGACGGG |
| | | TGAGTAACACGTGGGAACGTACCCTTCGGTTCGGAATAACGCTGGGA |
| | | AACTAGCGCTAATACCGGATACGCCCTTTTGGGGAAAGGTTTACTGCC |
| | | GAAGGATCGGCCCGCGTCTGATTAGCTAGTTGGTGGGGTAACGGCCT |
| | | ACCAAGGCGACGATCAGTAGCTGGTCTGAGAGGATGATCAGCCACAC |
| | | TGGGACTGAGACACGGCCCAGACTCCTACGGGAGGCAGCAGTGGGG |
| | | AATATTGGACAATGGGCGCAAGCCTGATCCAGCCATGCCGCGTGAGT |
| | | GATGAAGGCCTTAGGGTTGTAAAGCTCTTTTGTCCGGGACGATAATG |
| | | ACGGTACCGGAAGAATAAGCCCCGGCTAACTTCGTGCCAGCAGCCGC |
| | | GGTAATACGAAGGGGGCTAGCGTTGCTCGGAATCACTGGGCGTAAAG |
| | | GGCGCGTAGGCGGCCATTCAAGTCGGGGGTGAAAGCCTGTGGCTCAA |
| | | CCACAGAATTGCCTTCGATACTGTTTGGCTTGAGTTTGGTAGAGGTTG |
| | | GTGGAACTGCGAGTGTAGAGGTGAAATTCGTAGATATTCGCAAGAAC |
| | | ACCAGTGGCGAAGGCGGCCAACTGGACCAATACTGACGCTGAGGCGC |
| | | GAAAGCGTGGGGAGCAAACAGGATTAGATACCCTGGTAGTCCACGCC |
| | | GTAAACGATGAATGCTAGCTGTTGGGGTGCTTGCACCTCAGTAGCGC |
| | | AGCTAACGCTTTAAGCATTCCGCCTGGGGAGTACGGTCGCAAGATTA |
| | | AAACTCAAAGGAATTGACGGGGGCCCGCACAAGCGGTGGAGCATGT |
| | | GGTTTAATTCGAAGCAACGCGCAGAACCTTACCATCCCTTGACATGTC |
| | | GTGCCATCCGGAGAGATCCGGGGTTCCCTTCGGGGACGCGAACACAG |
| | | GTGCTGCATGGCTGTCGTCAGCTCGTGTCGTGAGATGTTGGGTTAAGT |
| | | CCCGCAACGAGCGCAACCCACGTCCTTAGTTGCCATCATTTAGTTGGG |
| | | CACTCTAGGGAGACTGCCGGTGATAAGCCGCGAGGAAGGTGTGGATG |
| | | ACGTC |
| 146 | DP81 16S rRNA | AACGGAGAGTTTGATCCTGGCTCAGGACGAACGCTGGCGGCGTGCCT |
| | | AATACATGCAAGTCGAGCGGACAGAAGGGAGCTTGCTCCCGGACGTT |
| | | AGCGGCGGACGGGTGAGTAACACGTGGGCAACCTGCCCCTTAGACTG |
| | | GGATAACTCCGGGAAACCGGAGCTAATACCGGATAATCCCTTTCTCC |
| | | ACCTGGAGAGAGGGTGAAAGATGGCTTCGGCTATCACTAGGGGATGG |
| | | GCCCGCGGCGCATTAGCTAGTTGGTAAGGTAACGGCTTACCAAGGCG |
| | | ACGATGCGTAGCCGACCTGAGAGGGTGATCGGCCACACTGGGACTGA |
| | | GACACGGCCCAGACTCCTACGGGAGGCAGCAGTAGGGAATCTTCCGC |
| | | AATGGACGAAAGTCTGACGGAGCAACGCCGCGTGAGTGAGGAAGGC |
| | | TTTCGGGTCGTAAAGCTCTGTTGTGAGGGAAGAAGCGGTACCGTTCG |
| | | AATAGGGCGGTACCTTGACGGTACCTCACCAGAAAGCCACGGCTAAC |
| | | TACGTGCCAGCAGCCGCGGTAATACGTAGGTGGCAAGCGTTGTCCGG |
| | | AATTATTGGGCGTAAAGCGCGCGCAGGCGGCTTCTTAAGTCTGATGT |
| | | GAAATCTCGGGGCTCAACCCCGAGCGGCCATTGGAAACTGGGGAGCT |
| | | TGAGTGCAGAAGAGGAGAGTGGAATTCCACGTGTAGCGGTGAAATGC |
| | | GTAGAGATGTGGAGGAACACCAGTGGCGAAGGCGACTCTCTGGTCTG |
| | | TAACTGACGCTGAGGCGCGAAAGCGTGGGGAGCAAACAGGATTAGA |
| | | TACCCTGGTAGTCCACGCCGTAAACGATGAGTGCTAGGTGTTAGGGG |
| | | TTTCGATGCCCGTAGTGCCGAAGTTAACACATTAAGCACTCCGCCTGG |
| | | GGAGTACGGCCGCAAGGCTGAAACTCAAAGGAATTGACGGGGACCC |
| | | GCACAAGCAGTGGAGCATGTGGTTTAATTCGAAGCAACGCGAAGAAC |
| | | CTTACCAGGTCTTGACATCCTTTGACCACCCAAGAGATTGGGCTTCCC |
| | | CTTCGGGGGCAAAGTGACAGGTGGTGCATGGTTGTCGTCAGCTCGTG |
| | | TCGTGAGATGTTGGGTTAAGTCCCGCAACGAGCGCAACCCTTGATCTT |
| | | AGTTGCCAGCATTGAGTTGGGCACTCTAAGGTGACTGCCGGTGACAA |
| | | ACCGGAGGAAGGTGGGGATGACGTCAAATCATCATGCCCCTTATGAC |
| | | CTGGGCTACACACGTGCTACAATGGATGGTACAAAGGGCAGCGAAAC |
| | | CGCGAGGTGAAGCCAATCCCATAAAGCCATTCTCAGTTCGGATTGCA |
| | | GGCTGCAACTCGCCTGCATGAAGCCGGAATTGCTAGTAATCGCGGAT |
| | | CAGCATGCCGCGGTGAATACGTTCCCGGGTCTTGTACACACCGCCCGT |
| | | CACACCACGAGAGTTTGTAACACCCGAAGTCGGTGAGGCAACCTTTT |
| | | GGAGCCAGCCGCCTAAGGTGGGACAAATGATTGGGGTGAAGTCGTAA |
| | | CAAGGTAGCCGTATCGGAAGGTGCGGCTGGATCACCTCCTTT |
| 147 | DP82 16S rRNA | AACGGAGAGTTTGATCCTGGCTCAGGACGAACGCTGGCGGCGTGCCT |
| | | AATACATGCAAGTCGAGCGGACAGAAGGGAGCTTGCTCCCGGACGTT |
| | | AGCGGCGGACGGGTGAGTAACACGTGGGCAACCTGCCCCTTAGACTG |
| | | GGATAACTCCGGGAAACCGGAGCTAATACCGGATAATCCCTTTCTCC |
| | | ACCTGGAGAGAGGGTGAAAGATGGCTTCGGCTATCACTAAGGGATGG |
| | | GCCCGCGGCGCATTAGCTAGTTGGTAAGGTAACGGCTTACCAAGGCA |
| | | ACGATGCGTAGCCGACCTGAGAGGGTGATCGGCCACACTGGGACTGA |
| | | GACACGGCCCAGACTCCTACGGGAGGCAGCAGTAGGGAATCTTCCGC |
| | | AATGGACGAAAGTCTGACGGAGCAACGCCGCGTGAGTGAGGAAGGC |
| | | CTTCGGGTCGTAAAGCTCTGTTGTGAGGGAAGAAGCGGTACCGTTCG |
| | | AATAGGGCGGTACCTTGACGGTACCTCACCAGAAAGCCACGGCTAAC |
| | | TACGTGCCAGCAGCCGCGGTAATACGTAGGTGGCAAGCGTTGTCCGG |
| | | AATTATTGGGCGTAAAGCGCGCGCAGGCGGCTTCTTAAGTCTGATGT |
| | | GAAATCTCGGGGCTCAACCCCGAGCGGCCATTGGAAACTGGGGAGCT |
| | | TGAGTGCAGAAGAGGAGAGTGGAATTCCACGTGTAGCGGTGAAATGC |
| | | GTAGAGATGTGGAGGAACACCAGTGGCGAAGGCGACTCTCTGGTCTG |
| | | TAACTGACGCTGAGGCGCGAAAGCGTGGGGAGCAAACAGGATTAGA |
| | | TACCCTGGTAGTCCACGCCGTAAACGATGAGTGCTAGGTGTTAGGGG |
| | | TTTCGATGCCCGTAGTGCCGAAGTTAACACATTAAGCACTCCGCCTGG |
| | | GGAGTACGGCCGCAAGGCTGAAACTCAAAGGAATTGACGGGGACCC |
| | | GCACAAGCAGTGGAGCATGTGGTTTAATTCGAAGCAACGCGAAGAAC |
| | | CTTACCAGGTCTTGACATCCTTTGACCACCCAAGAGATTGGGCTTCCC |
| | | CTTCGGGGGCAAAGTGACAGGTGGTGCATGGTTGTCGTCAGCTCGTG |
| | | TCGTGAGATGTTGGGTTAAGTCCCGCAACGAGCGCAACCCTTGATCTT |
| | | AGTTGCCAGCATTCAGTTGGGCACTCTAAGGTGACTGCCGGTGACAA |
| | | ACCGGAGGAAGGTGGGGATGACGTCAAATCATCATGCCCCTTATGAC |
| | | CTGGGCTACACACGTGCTACAATGGATGGTACAAAGGGCAGCGAAAC |
| | | CGCGAGGTGAAGCCAATCCCATAAAGCCATTCTCAGTTCGGATTGCA |
| | | GGCTGCAACTCGCCTGCATGAAGCCGGAATTGCTAGTAATCGCGGAT |
| | | CAGCATGCCGCGGTGAATACGTTCCCGGGTCTTGTACACACCGCCCGT |
| | | CACACCACGAGAGTTTGTAACACCCGAAGTCGGTGAGGCAACCTTTT |
| | | GGAGCCAGCCGCCTAAGGTGGGACAAATGATTGGGGTGAAGTCGTAA |
| | | CAAGGTAGCCGTATCGGAAGGTGCGGCTGGATCACCTCCTTT |
| 148 | DP83 16S rRNA | ACGGAGAGTTTGATCCTGGCTCAGGACGAACGCTGGCGGCGTGCCTA |
| | | ATACATGCAAGTCGAGCGGAGTTTCAAGAAGCTTGCTTTTTGAAACTT |
| | | AGCGGCGGACGGGTGAGTAACACGTGGGCAACCTGCCCCTTAGACTG |
| | | GGATAACTCCGGGAAACCGGAGCTAATACCGGATAATCCCTTTCTCC |
| | | ACCTGGAGAGAGGGTGAAAGATGGCTTCGGCTATCACTAAGGGATGG |
| | | GCCCGCGGCGCATTAGCTAGTTGGTAAGGTAACGGCTTACCAAGGCA |
| | | ACGATGCGTAGCCGACCTGAGAGGGTGATCGGCCACACTGGGACTGA |
| | | GACACGGCCCAGACTCCTACGGGAGGCAGCAGTAGGGAATCTTCCGC |
| | | AATGGACGAAAGTCTGACGGAGCAACGCCGCGTGAGTGAGGAAGGC |
| | | CTTCGGGTCGTAAAGCTCTGTTGTGAGGGAAGAAGCGGTACCGTTCG |
| | | AATAGGGCGGTACCTTGACGGTACCTCACCAGAAAGCCACGGCTAAC |
| | | TACGTGCCAGCAGCCGCGGTAATACGTAGGTGGCAAGCGTTGTCCGG |
| | | AATTATTGGGCGTAAAGCGCGCGCAGGCGGCTTCTTAAGTCTGATGT |
| | | GAAATCTCGGGGCTCAACCCCGAGCGGCCATTGGAAACTGGGGAGCT |
| | | TGAGTGCAGAAGAGGAGAGTGGAATTCCACGTGTAGCGGTGAAATGC |
| | | GTAGAGATGTGGAGGAACACCAGTGGCGAAGGCGACTCTCTGGTCTG |
| | | TAACTGACGCTGAGGCGCGAAAGCGTGGGGAGCAAACAGGATTAGA |
| | | TACCCTGGTAGTCCACGCCGTAAACGATGAGTGCTAGGTGTTAGGGG |
| | | TTTCGATGCCCGTAGTGCCGAAGTTAACACATTAAGCACTCCGCCTGG |
| | | GGAGTACGGCCGCAAGGCTGAAACTCAAAGGAATTGACGGGGACCC |
| | | GCACAAGCAGTGGAGCATGTGGTTTAATTCGAAGCAACGCGAAGAAC |
| | | CTTACCAGGTCTTGACATCCTTTGACCACCCAAGAGATTGGGCTTCCC |
| | | CTTCGGGGGCAAAGTGACAGGTGGTGCATGGTTGTCGTCAGCTCGTG |
| | | TCGTGAGATGTTGGGTTAAGTCCCGCAACGAGCGCAACCCTTGATCTT |
| | | AGTTGCCAGCATTCAGTTGGGCACTCTAAGGTGACTGCCGGTGACAA |
| | | ACCGGAGGAAGGTGGGGATGACGTCAAATCATCATGCCCCTTATGAC |
| | | CTGGGCTACACACGTGCTACAATGGATGGTACAAAGGGCAGCGAAGC |
| | | CGCGAGGTGAAGCCAATCCCATAAAGCCATTCTCAGTTCGGATTGCA |
| | | GGCTGCAACTCGCCTGCATGAAGCCGGAATTGCTAGTAATCGCGGAT |
| | | CAGCATGCCGCGGTGAATACGTTCCCGGGTCTTGTACACACCGCCCGT |
| | | CACACCACGAGAGTTTGTAACACCCGAAGTCGGTGAGGCAACCTTTT |
| | | GGAGCCAGCCGCCTAAGGTGGGACAAATGATTGGGGTGAAGTCGTAA |
| | | CAAGGTAGCCGTATCGGAAGGTGCGGCTGGATCACCTCCTTT |
| 149 | DP84 16S rRNA | TACGGAGAGTTTGATCCTGGCTCAGGATGAACGCTGGCGGCGTGCTT |
| | | AACACATGCAAGTCGAACGGTGAAGCCAAGCTTGCTTGGTGGATCAG |
| | | TGGCGAACGGGTGAGTAACACGTGAGCAACCTGCCCTGGACTCTGGG |
| | | ATAAGCGCTGGAAACGGCGTCTAATACTGGATATGAGCTCTCATCGC |
| | | ATGGTGGGGGTTGGAAAGATTTTTTGGTCTGGGATGGGCTCGCGGCCT |
| | | ATCAGCTTGTTGGTGAGGTAATGGCTCACCAAGGCGTCGACGGGTAG |
| | | CCGGCCTGAGAGGGTGACCGGCCACACTGGGACTGAGACACGGCCCA |
| | | GACTCCTACGGGAGGCAGCAGTGGGGAATATTGCACAATGGGCGAAA |
| | | GCCTGATGCAGCAACGCCGCGTGAGGGATGACGGCCTTCGGGTTGTA |
| | | AACCTCTTTTAGCAGGGAAGAAGCGAAAGTGACGGTACCTGCAGAAA |
| | | AAGCGCCGGCTAACTACGTGCCAGCAGCCGCGGTAATACGTAGGGCG |
| | | CAAGCGTTATCCGGAATTATTGGGCGTAAAGAGCTCGTAGGCGGTTT |
| | | GTCGCGTCTGCTGTGAAATCCCGAGGCTCAACCTCGGGCCTGCAGTG |
| | | GGTACGGGCAGACTAGAGTGCGGTAGGGGAGATTGGAATTCCTGGTG |
| | | TAGCGGTGGAATGCGCAGATATCAGGAGGAACACCGATGGCGAAGG |
| | | CAGATCTCTGGGCCGTAACTGACGCTGAGGAGCGAAAGGGTGGGGAG |
| | | CAAACAGGCTTAGATACCCTGGTAGTCCACCCCGTAAACGTTGGGAA |
| | | CTAGTTGTGGGGACCATTCCACGGTTTCCGTGACGCAGCTAACGCATT |
| | | AAGTTCCCCGCCTGGGGAGTACGGCCGCAAGGCTAAAACTCAAAGGA |
| | | ATTGACGGGGACCCGCACAAGCGGCGGAGCATGCGGATTAATTCGAT |
| | | GCAACGCGAAGAACCTTACCAAGGCTTGACATACACCAGAACGGGCC |
| | | AGAAATGGTCAACTCTTTGGACACTGGTGAACAGGTGGTGCATGGTT |
| | | GTCGTCAGCTCGTGTCGTGAGATGTTGGGTTAAGTCCCGCAACGAGC |
| | | GCAACCCTCGTTCTATGTTGCCAGCACGTAATGGTGGGAACTCATGGG |
| | | ATACTGCCGGGGTCAACTCGGAGGAAGGTGGGGATGACGTCAAATCA |
| | | TCATGCCCCTTATGTCTTGGGCTTCACGCATGCTACAATGGCCGGTAC |
| | | AAAGGGCTGCAATACCGTGAGGTGGAGCGAATCCCAAAAAGCCGGTC |
| | | CCAGTTCGGATTGAGGTCTGCAACTCGACCTCATGAAGTCGGAGTCG |
| | | CTAGTAATCGCAGATCAGCAACGCTGCGGTGAATACGTTCCCGGGTC |
| | | TTGTACACACCGCCCGTCAAGTCATGAAAGGAGCCGTCGAAGGTGGG |
| | | ATCGGTAATTAGGACTAAGTCGTAACAAGGTAGCCGTACCGGAAGGT |
| | | GCGGCTGGATCACCTCCTTT |
| 150 | DP85 16S rRNA | ACGGTCGGGGGCATCAGTATTCAGTCGTCAGAGGTGAAATTCTTGGA |
| | | TTGACTGAAGACTAACTACTGCGAAAGCATTTGCCAAGGACGTTTTCA |
| | | TTAATCAAGAACGAAAGTTAGGGGATCGAAGATGATCAGATACCGTC |
| | | GTAGTCTTAACCATAAACTATGCCGACTAGAGATCGGGTGGTGCTTTT |
| | | TGCGCACTCGGCATCTTACGAGAAATCAAAGTCTTTGGGTTCTGGGGG |
| | | GAGTATGGTCGCAAGGCTGAAACTTAAAGGAATTGACGGAGGGGCAC |
| | | CACCAGGAGTGGAGCCTGCGGCTTAATTTGACTCAACACGGGGAAAC |
| | | TCACCAGGTCCAGACGTAATAAGGATTGACAAGTTAGAGACTTCTCTT |
| | | GATCTTACGGGTGGTGGTGCATGGCCGTTTTTAGTCCTTGGAGTGATT |
| | | TGTCTGCTTAATTGCGATAACGGACGAGACCTTAACCTGCTAAATAGG |
| | | GCTGCGAGCATCTGCTCGTGGGCTCTTCTTAGAGGGACTATGGGTATC |
| | | AAACCCATGGAAGTTTGAGGCAACAACAGGTCTGTGATGCCCTTAGA |
| | | CGTTCTGGGCCGCACGCGCGCTACACTGACGGAGCCAGCAAGCATAA |
| | | CCTTGGTCGAGAGGCCTGGGTAATCTCGTGAAACTCCGTCGTGCTGGG |
| | | GATAGAGCATTGTAATTTTTGCTCTTCAACGAGGAATTCCTAGTAAGC |
| | | GCAAGTCATCAGCTTGCGTTGATTACGTCCCTGCCCCTTGTACACACC |
| | | GCCCGTCGCTACTACCGATTGAATGGCTTAGTGAGGCTTCAAGACCG |
| | | GCGCGGCCTGCGGGGCAACTCGCGCGCTGCGCTGGGAATTTAGTCAA |
| | | ACTTGGTCATTTAGAGGTCGTAAAAGTCGTAACAAGGTTTCCGTAGGT |
| | | GAACCTGCGGAAGGATCATT |
| 151 | DP86 16S rRNA | CGATGCGTAGCCGACCTGAGAGGGTGATCGGCCACACTGGGACTGAG |
| | | ACACGGCCCAGACTCCTACGGGAGGCAGCAGTAGGGAATCTTCCGCA |
| | | ATGGACGAAAGTCTGACGGAGCAACGCCGCGTGAGTGATGAAGGTTT |
| | | TCGGATCGTAAAGCTCTGTTGTTAGGGAAGAACAAGTGCCGTTCAAA |
| | | TAGGGCGGCACCTTGACGGTACCTAACCAGAAAGCCACGGCTAACTA |
| | | CGTGCCAGCAGCCGCGGTAATACGTAGGTGGCAAGCGTTGTCCGGAA |
| | | TTATTGGGCGTAAAGGGCTCGCAGGCGGTTTCTTAAGTCTGATGTGAA |
| | | AGCCCCCGGCTCAACCGGGGAGGGTCATTGGAAACTGGGGAACTTGA |
| | | GTGCAGAAGAGGAGAGTGGAATTCCACGTGTAGCGGTGAAATGCGTA |
| | | GAGATGTGGAGGAACACCAGTGGCGAAGGCGACTCTCTGGTCTGTAA |
| | | CTGACGCTGAGGAGCGAAAGCGTGGGGAGCGAACAGGATTAGATAC |
| | | CCTGGTAGTCCACGCCGTAAACGATGAGTGCTAAGTGTTAGGGGGTT |
| | | TCCGCCCCTTAGTGCTGCAGCTAACGCATTAAGCACTCCGCCTGGGGA |
| | | GTACGGTCGCAAGACTGAAACTCAAAGGAATTGACGGGGGCCCGCAC |
| | | AAGCGGTGGAGCATGTGGTTTAATTCGAAGCAACGCGAAGAACCTTA |
| | | CCAGGTCTTGACATCCTCTGACAATCCTAGAGATAGGACGTCCCCTTC |
| | | GGGGGCAGAGTGACAGGTGGTGCATGGTTGTCGTCAGCTCGTGTCGT |
| | | GAGATGTTGGGTTAAGTCCCGCAACGAGCGCAACCCTTGATCTTAGTT |
| | | GCCAGCATTCAGTTGGGTGTTCTTTGAAAACT |
| 152 | DP87 16S rRNA | TTTGAGAGTTTGATCCTGGCTCAGGACGAACGCTGGCGGCGTGCCTA |
| | | ATACATGCAAGTCGAACGAACTCTGGTATTGATTGGTGCTTGCATCAT |
| | | GATTTACATTTGAGTGAGTGGCGAACTGGTGAGTAACACGTGGGAAA |
| | | CCTGCCCAGAAGCGGGGGATAACACCTGGAAACAGATGCTAATACCG |
| | | CATAACAACTTGGACCGCATGGTCCGAGCTTGAAAGATGGCTTCGGC |
| | | TATCACTTTTGGATGGTCCCGCGGCGTATTAGCTAGATGGTGGGGTAA |
| | | CGGCTCACCATGGCAATGATACGTAGCCGACCTGAGAGGGTAATCGG |
| | | CCACATTGGGACTGAGACACGGCCCAAACTCCTACGGGAGGCAGCAG |
| | | TAGGGAATCTTCCACAATGGACGAAAGTCTGATGGAGCAACGCCGCG |
| | | TGAGTGAAGAAGGGTTTCGGCTCGTAAAACTCTGTTGTTAAAGAAGA |
| | | ACATATCTGAGAGTAACTGTTCAGGTATTGACGGTATTTAACCAGAA |
| | | AGCCACGGCTAACTACGTGCCAGCAGCCGCGGTAATACGTAGGTGGC |
| | | AAGCGTTGTCCGGATTTATTGGGCGTAAAGCGAGCGCAGGCGGTTTTT |
| | | TAAGTCTGATGTGAAAGCCTTCGGCTCAACCGAAGAAGTGCATCGGA |
| | | AACTGGGAAACTTGAGTGCAGAAGAGGACAGTGGAACTCCATGTGTA |
| | | GCGGTGAAATGCGTAGATATATGGAAGAACACCAGTGGCGAAGGCG |
| | | GCTGTCTGGTCTGTAACTGACGCTGAGGCTCGAAAGTATGGGTAGCA |
| | | AACAGGATTAGATACCCTGGTAGTCCATACCGTAAACGATGAATGCT |
| | | AAGTGTTGGAGGGTTTCCGCCCTTCAGTGCTGCAGCTAACGCATTAAG |
| | | CATTCCGCCTGGGGAGTACGGCCGCAAGGCTGAAACTCAAAGGAATT |
| | | GACGGGGGCCCGCACAAGCGGTGGAGCATGTGGTTTAATTCGAAGCT |
| | | ACGCGAAGAACCTTACCAGGTCTTGACATACTATGCAAATCTAAGAG |
| | | ATTAGACGTTCCCTTCGGGGACATGGATACAGGTGGTGCATGGTTGTC |
| | | GTCAGCTCGTGTCGTGAGATGTTGGGTTAAGTCCCGCAACGAGCGCA |
| | | ACCCTTATTATCAGTTGCCAGCATTAAGTTGGGCACTCTGGTGAGACT |
| | | GCCGGTGACAAACCGGAGGAAGGTGGGGATGACGTCAAATCATCATG |
| | | CCCCTTATGACCTGGGCTACACACGTGCTACAATGGATGGTACAACG |
| | | AGTTGCGAACTCGCGAGAGTAAGCTAATCTCTTAAAGCCATTCTCAGT |
| | | TCGGATTGTAGGCTGCAACTCGCCTACATGAAGTCGGAATCGCTAGT |
| | | AATCGCGGATCAGCATGCCGCGGTGAATACGTTCCCGGGCCTTGTAC |
| | | ACACCGCCCGTCACACCATGAGAGTTTGTAACACCCAAAGTCGGTGG |
| | | GGTAACCTTTTAGGAACCAGCCGCCTAAGGTGGGACAGATGATTAGG |
| | | GTGAAGTCGTAACAAGGTAGCCGTAGGAGAACCTGCGGCTGGATCAC |
| | | CTCCTT |
| 153 | DP88 16S rRNA | TAGTGGGTTTGATCCTGGCTCAGGACGAACGCTGGCGGCGTGCCTAA |
| | | TACATGCAAGTCGAGCGGACAGATGGGAGCTTGCTCCCTGATGTTAG |
| | | CGGCGGACGGGTGAGTAACACGTGGGTAACCTGCCTGTAAGACTGGG |
| | | ATAACTCCGGGAAACCGGGGCTAATACCGGATGGTTGTCTGAACCGC |
| | | ATGGTTCAGACATAAAAGGTGGCTTCGGCTACCACTTACAGATGGAC |
| | | CCGCGGCGCATTAGCTAGTTGGTGAGGTAACGGCTCACCAAGGCGAC |
| | | GATGCGTAGCCGACCTGAGAGGGTGATCGGCCACACTGGGACTGAGA |
| | | CACGGCCCAGACTCCTACGGGAGGCAGCAGTAGGGAATCTTCCGCAA |
| | | TGGACGAAAGTCTGACGGAGCAACGCCGCGTGAGTGATGAAGGTTTT |
| | | CGGATCGTAAAGCTCTGTTGTTAGGGAAGAACAAGTGCCGTTCAAAT |
| | | AGGGCGGCACCTTGACGGTACCTAACCAGAAAGCCACGGCTAACTAC |
| | | GTGCCAGCAGCCGCGGTAATACGTAGGTGGCAAGCGTTGTCCGGAAT |
| | | TATTGGGCGTAAAGGGCTCGCAGGCGGTTTCTTAAGTCTGATGTGAA |
| | | AGCCCCCGGCTCAACCGGGGAGGGTCATTGGAAACTGGGGAACTTGA |
| | | GTGCAGAAGAGGAGAGTGGAATTCCACGTGTAGCGGTGAAATGCGTA |
| | | GAGATGTGGAGGAACACCAGTGGCGAAGGCGACTCTCTGGTCTGTAA |
| | | CTGACGCTGAGGAGCGAAAGCGTGGGGAGCGAACAGGATTAGATAC |
| | | CCTGGTAGTCCACGCCGTAAACGATGAGTGCTAAGTGTTAGGGGGTT |
| | | TCCGCCCCTTAGTGCTGCAGCTAACGCATTAAGCACTCCGCCTGGGGA |
| | | GTACGGTCGCAAGACTGAAACTCAAAGGAATTGACGGGGGCCCGCAC |
| | | AAGCGGTGGAGCATGTGGTTTAATTCGAAGCAACGCGAAGAACCTTA |
| | | CCAGGTCTTGACATCCTCTGACAATCCTAGAGATAGGACGTCCCCTTC |
| | | GGGGGCAGAGTGACAGGTGGTGCATGGTTGTCGTCAGCTCGTGTCGT |
| | | GAGATGTTGGGTTAAGTCCCGCAACGAGCGCAACCCTTGATCTTAGTT |
| | | GCCAGCATTCAGTTGGGCACTCTAAGGTGACTGCCGGTGACAAACCG |
| | | GAGGAAGGTGGGGATGACGTCAAATCATCATGCCCCTTATGACCTGG |
| | | GCTACACACGTGCTACAATGGACAGAACAAAGGGCAGCGAAACCGC |
| | | GAGGTTAAGCCAATCCCACAAATCTGTTCTCAGTTCGGATCGCAGTCT |
| | | GCAACTCGACTGCGTGAAGCTGGAATCGCTAGTAATCGCGGATCAGC |
| | | ATGCCGCGGTGAATACGTTCCCGGGCCTTGTACACACCGCCCGTCACA |
| | | CCACGAGAGTTTGTAACACCCGAAGTCGGTGAGGTAACCTTTATGGA |
| | | GCCAGCCGCCGAAGGTGGGACAGATGATTGGGGTGAAGTCGTAACAA |
| | | GGTAGCCGTATCGGAAGGTGCGGCTGGATCACCTCCTTT |
| 154 | DP89 16S rRNA | GTAACGGCTCACCAAGGCAACGATGCGTAGCCGACCTGAGAGGGTGA |
| | | TCGGCCACACTGGGACTGAGACACGGCCCAGACTCCTACGGGAGGCA |
| | | GCAGTAGGGAATCTTCCGCAATGGACGAAAGTCTGACGGAGCAACGC |
| | | CGCGTGAGTGATGAAGGTTTTCGGATCGTAAAGCTCTGTTGTTAGGGA |
| | | AGAACAAGTACCGTTCGAATAGGGCGGTACCTTGACGGTACCTAACC |
| | | AGAAAGCCACGGCTAACTACGTGCCAGCAGCCGCGGTAATACGTAGG |
| | | TGGCAAGCGTTGTCCGGAATTATTGGGCGTAAAGGGCTCGCAGGCGG |
| | | TTTCTTAAGTCTGATGTGAAAGCCCCCGGCTCAACCGGGGAGGGTCAT |
| | | TGGAAACTGGGGAACTTGAGTGCAGAAGAGGAGAGTGGAATTCCAC |
| | | GTGTAGCGGTGAAATGCGTAGAGATGTGGAGGAACACCAGTGGCGA |
| | | AGGCGACTCTCTGGTCTGTAACTGACGCTGAGGAGCGAAAGCGTGGG |
| | | GAGCGAACAGGATTAGATACCCTGGTAGTCCACGCCGTAAACGATGA |
| | | GTGCTAAGTGTTAGGGGGTTTCCGCCCCTTAGTGCTGCAGCTAACGCA |
| | | TTAAGCACTCCGCCTGGGGAGTACGGTCGCAAGACTGAAACTCAAAG |
| | | GAATTGACGGGGGCCCGCACAAGCGGTGGAGCATGTGGTTTAATTCG |
| | | AAGCAACGCGAAGAACCTTACCAGGTCTTGACATCCTCTGACAATCC |
| | | TAGAGATAGGACGTCCCCTTCGGGGGCAGAGTGACAGGTGGTGCATG |
| | | GTTGTCGTCAGCTCGTGTCGTGAGATGTTGGGTTAAGTCCCGCAACGA |
| | | GCGCAACCCTTGATCTTAGTTGCCAGCATTCAGTTGGGCACTCTAAGG |
| | | TGACTGCCGGTGACAAACCGGAGGAAGGTGGGGATGACGTCAAATCA |
| | | TCATGCCCCTTATGACCTGGGCTACACACGTGCTACAATGGACAGAA |
| | | CAAAGGGCAGCGAAACCGCGAGGTTAAGCCAATCCCACAAATCTGTT |
| | | CTCAGTTCGGATCGCAGTCTGCAACTCGACTGCGTGAAGCTGGAATC |
| | | GCTAGTAATCGCGGATCAGCATGCCGCGGTGAATACGTTCCCGGGCC |
| | | TTGTACACACCGCCCGTCACACCACGAGAGTTTGTAACACCCGAAGT |
| | | CGGTGAGGTAACCTTTTAGGAGCCAGCCGCCGAAGGTGGGACAGATG |
| | | ATTGGGGTGAAGTCGTAACAAGGTAGCCGTATCGGAAGGTGCGGCTG |
| | | GATCACCTCCTTT |
| 155 | DP90 16S rRNA | TTTGAGAGTTTGATCCTGGCTCAGGACGAACGCTGGCGGCGTGCCTA |
| | | ATACATGCAAGTCGAACGAACTCTGGTATTGATTGGTGCTTGCATCAT |
| | | GATTTACATTTGAGTGAGTGGCGAACTGGTGAGTAACACGTGGGAAA |
| | | CCTGCCCAGAAGCGGGGGATAACACCTGGAAACAGATGCTAATACCG |
| | | CATAACAACTTGGACCGCATGGTCCGAGCTTGAAAGATGGCTTCGGC |
| | | TATCACTTTTGGATGGTCCCGCGGCGTATTAGCTAGATGGTGGGGTAA |
| | | CGGCTCACCATGGCAATGATACGTAGCCGACCTGAGAGGGTAATCGG |
| | | CCACATTGGGACTGAGACACGGCCCAAACTCCTACGGGAGGCAGCAG |
| | | TAGGGAATCTTCCACAATGGACGAAAGTCTGATGGAGCAACGCCGCG |
| | | TGAGTGAAGAAGGGTTTCGGCTCGTAAAACTCTGTTGTTAAAGAAGA |
| | | ACATATCTGAGAGTAACTGTTCAGGTATTGACGGTATTTAACCAGAA |
| | | AGCCACGGCTAACTACGTGCCAGCAGCCGCGGTAATACGTAGGTGGC |
| | | AAGCGTTGTCCGGATTTATTGGGCGTAAAGCGAGCGCAGGCGGTTTTT |
| | | TAAGTCTGATGTGAAAGCCTTCGGCTCAACCGAAGAAGTGCATCGGA |
| | | AACTGGGAAACTTGAGTGCAGAAGAGGACAGTGGAACTCCATGTGTA |
| | | GCGGTGAAATGCGTAGATATATGGAAGAACACCAGTGGCGAAGGCG |
| | | GCTGTCTGGTCTGTAACTGACGCTGAGGCTCGAAAGTATGGGTAGCA |
| | | AACAGGATTAGATACCCTGGTAGTCCATACCGTAAACGATGAATGCT |
| | | AAGTGTTGGAGGGTTTCCGCCCTTCAGTGCTGCAGCTAACGCATTAAG |
| | | CATTCCGCCTGGGGAGTACGGCCGCAAGGCTGAAACTCAAAGGAATT |
| | | GACGGGGGCCCGCACAAGCGGTGGAGCATGTGGTTTAATTCGAAGCT |
| | | ACGCGAAGAACCTTACCAGGTCTTGACATACTATGCAAATCTAAGAG |
| | | ATTAGACGTTCCCTTCGGGGACATGGATACAGGTGGTGCATGGTTGTC |
| | | GTCAGCTCGTGTCGTGAGATGTTGGGTTAAGTCCCGCAACGAGCGCA |
| | | ACCCTTATTATCAGTTGCCAGCATTAAGTTGGGCACTCTGGTGAGACT |
| | | GCCGGTGACAAACCGGAGGAAGGTGGGGATGACGTCAAATCATCATG |
| | | CCCCTTATGACCTGGGCTACACACGTGCTACAATGGATGGTACAACG |
| | | AGTTGCGAACTCGCGAGAGTAAGCTAATCTCTTAAAGCCATTCTCAGT |
| | | TCGGATTGTAGGCTGCAACTCGCCTACATGAAGTCGGAATCGCTAGT |
| | | AATCGCGGATCAGCATGCCGCGGTGAATACGTTCCCGGGCCTTGTAC |
| | | ACACCGCCCGTCACACCATGAGAGTTTGTAACACCCAAAGTCGGTGG |
| | | GGTAACCTTTTAGGAACCAGCCGCCTAAGGTGGGACAGATGATTAGG |
| | | GTGAAGTCGTAACAAGGTAGCCGTAGGAGAACCTGCGGCTGGATCAC |
| | | CTCCTT |
| 156 | DP92 16S rRNA | CGATGCGTAGCCGACCTGAGAGGGTGATCGGCCACACTGGGACTGAG |
| | | ACACGGCCCAGACTCCTACGGGAGGCAGCAGTAGGGAATCTTCCGCA |
| | | ATGGACGAAAGTCTGACGGAGCAACGCCGCGTGAGTGATGAAGGTTT |
| | | TCGGATCGTAAAGCTCTGTTGTTAGGGAAGAACAAGTACCGTTCGAA |
| | | TAGGGCGGTACCTTGACGGTACCTAACCAGAAAGCCACGGCTAACTA |
| | | CGTGCCAGCAGCCGCGGTAATACGTAGGTGGCAAGCGTTGTCCGGAA |
| | | TTATTGGGCGTAAAGGGCTCGCAGGCGGTTTCTTAAGTCTGATGTGAA |
| | | AGCCCCCGGCTCAACCGGGGAGGGTCATTGGAAACTGGGGAACTTGA |
| | | GTGCAGAAGAGGAGAGTGGAATTCCACGTGTAGCGGTGAAATGCGTA |
| | | GAGATGTGGAGGAACACCAGTGGCGAAGGCGACTCTCTGGTCTGTAA |
| | | CTGACGCTGAGGAGCGAAAGCGTGGGGAGCGAACAGGATTAGATAC |
| | | CCTGGTAGTCCACGCCGTAAACGATGAGTGCTAAGTGTTAGGGGGTT |
| | | TCCGCCCCTTAGTGCTGCAGCTAACGCATTAAGCACTCCGCCTGGGGA |
| | | GTACGGTCGCAAGACTGAAACTCAAAGGAATTGACGGGGGCCCGCAC |
| | | AAGCGGTGGAGCATGTGGTTTAATTCGAAGCAACGCGAAGAACCTTA |
| | | CCAGGTCTTGACATCCTCTGACAATCCTAGAGATAGGACGTCCCCTTC |
| | | GGGGGCAGAGTGACAGGTGGTGCATGGTTGTCGTCAGCTCGTGTCGT |
| | | GAGATGTTGGGTTAAGTCCCGCAACGAGCGCAACCCTTGATCTTAGTT |
| | | GCCAGCATTCAGTTGGGCACTCTAAGGTGACTGCCGGTGACAAACCG |
| | | GAGGAAGGTGGGGATGACGTCAAATCATCATGCCCCTTATGACCTGG |
| | | GCTACACACGTGCTACAATGGACAGAACAAAGGGCAGCGAAACCGC |
| | | GAGGTTAAGCCAATCCCACAAATCTGTTCTCAGTTCGGATCGCAGTCT |
| | | GCAACTCGACTGCGTGAAGCTGGAATCGCTAGTAATCGCGGATCAGC |
| | | ATGCCGCGGTGAATACGTTCCCGGGCCTTGTACACACCGCCCGTCACA |
| | | CCACGAGAGTTTGTAACACCCGAAGTCGGTGAGGTAACCTTTTAGGA |
| | | GCCAGCCGCCGAAGGTGGGACAGATGATTGGGGTGAAGTCGTAACAA |
| | | GGTAGCCGTATCGGAAGGTGCGGCTGGATCACCTCCTTT |
| 157 | DP93 16S rRNA | ATTGAGAGTTTGATCCTGGCTCAGGATGAACGCTGGCGGCGTGCCTA |
| | | ATACATGCAAGTCGAACGCACAGCGAAAGGTGCTTGCACCTTTCAAG |
| | | TGAGTGGCGAACGGGTGAGTAACACGTGGACAACCTGCCTCAAGGCT |
| | | GGGGATAACATTTGGAAACAGATGCTAATACCGAATAAAACTTAGTG |
| | | TCGCATGACAAAAAGTTAAAAGGCGCTTCGGCGTCACCTAGAGATGG |
| | | ATCCGCGGTGCATTAGTTAGTTGGTGGGGTAAAGGCCTACCAAGACA |
| | | ATGATGCATAGCCGAGTTGAGAGACTGATCGGCCACATTGGGACTGA |
| | | GACACGGCCCAAACTCCTACGGGAGGCTGCAGTAGGGAATCTTCCAC |
| | | AATGGGCGAAAGCCTGATGGAGCAACGCCGCGTGTGTGATGAAGGCT |
| | | TTCGGGTCGTAAAGCACTGTTGTATGGGAAGAACAGCTAGAATAGGA |
| | | AATGATTTTAGTTTGACGGTACCATACCAGAAAGGGACGGCTAAATA |
| | | CGTGCCAGCAGCCGCGGTAATACGTATGTCCCGAGCGTTATCCGGATT |
| | | TATTGGGCGTAAAGCGAGCGCAGACGGTTTATTAAGTCTGATGTGAA |
| | | AGCCCGGAGCTCAACTCCGGAATGGCATTGGAAACTGGTTAACTTGA |
| | | GTGCAGTAGAGGTAAGTGGAACTCCATGTGTAGCGGTGGAATGCGTA |
| | | GATATATGGAAGAACACCAGTGGCGAAGGCGGCTTACTGGACTGCAA |
| | | CTGACGTTGAGGCTCGAAAGTGTGGGTAGCAAACAGGATTAGATACC |
| | | CTGGTAGTCCACACCGTAAACGATGAACACTAGGTGTTAGGAGGTTT |
| | | CCGCCTCTTAGTGCCGAAGCTAACGCATTAAGTGTTCCGCCTGGGGAG |
| | | TACGACCGCAAGGTTGAAACTCAAAGGAATTGACGGGGACCCGCACA |
| | | AGCGGTGGAGCATGTGGTTTAATTCGAAGCAACGCGAAGAACCTTAC |
| | | CAGGTCTTGACATCCTTTGAAGCTTTTAGAGATAGAAGTGTTCTCTTC |
| | | GGAGACAAAGTGACAGGTGGTGCATGGTCGTCGTCAGCTCGTGTCGT |
| | | GAGATGTTGGGTTAAGTCCCGCAACGAGCGCAACCCTTATTGTTAGTT |
| | | GCCAGCATTCAGATGGGCACTCTAGCGAGACTGCCGGTGACAAACCG |
| | | GAGGAAGGCGGGGACGACGTCAGATCATCATGCCCCTTATGACCTGG |
| | | GCTACACACGTGCTACAATGGCGTATACAACGAGTTGCCAACCCGCG |
| | | AGGGTGAGCTAATCTCTTAAAGTACGTCTCAGTTCGGATTGTAGTCTG |
| | | CAACTCGACTACATGAAGTCGGAATCGCTAGTAATCGCGGATCAGCA |
| | | CGCCGCGGTGAATACGTTCCCGGGTCTTGTACACACCGCCCGTCACAC |
| | | CATGGGAGTTTGTAATGCCCAAAGCCGGTGGCCTAACCTTTTAGGAA |
| | | GGAGCCGTCTAAGGCAGGACAGATGACTGGGGTGAAGTCGTAACAA |
| | | GGTAGCCGTAGGAGAACCTGCGGCTGGATCACCTCCTTT |
| 158 | DP94 16S rRNA | ATCTGCCCAGAAGCAGGGGATAACACTTGGAAACAGGTGCTAATACC |
| | | GTATAACAACAAAATCCGCATGGATTTTGTTTGAAAGGTGGCTTCGGC |
| | | TATCACTTCTGGATGATCCCGCGGCGTATTAGTTAGTTGGTGAGGTAA |
| | | AGGCCCACCAAGACGATGATACGTAGCCGACCTGAGAGGGTAATCGG |
| | | CCACATTGGGACTGAGACACGGCCCAAACTCCTACGGGAGGCAGCAG |
| | | TAGGGAATCTTCCACAATGGACGAAAGTCTGATGGAGCAATGCCGCG |
| | | TGAGTGAAGAAGGGTTTCGGCTCGTAAAACTCTGTTGTTAAAGAAGA |
| | | ACACCTTTGAGAGTAACTGTTCAAGGGTTGACGGTATTTAACCAGAA |
| | | AGCCACGGCTAACTACGTGCCAGCAGCCGCGGTAATACGTAGGTGGC |
| | | AAGCGTTGTCCGGATTTATTGGGCGTAAAGCGAGCGCAGGCGGTTTTT |
| | | TAAGTCTGATGTGAAAGCCTTCGGCTTAACCGGAGAAGTGCATCGGA |
| | | AACTGGGAGACTTGAGTGCAGAAGAGGACAGTGGAACTCCATGTGTA |
| | | GCGGTGGAATGCGTAGATATATGGAAGAACACCAGTGGCGAAGGCG |
| | | GCTGTCTAGTCTGTAACTGACGCTGAGGCTCGAAAGCATGGGTAGCG |
| | | AACAGGATTAGATACCCTGGTAGTCCATGCCGTAAACGATGAGTGCT |
| | | AAGTGTTGGAGGGTTTCCGCCCTTCAGTGCTGCAGCTAACGCATTAAG |
| | | CACTCCGCCTGGGGAGTACGACCGCAAGGTTGAAACTCAAAGGAATT |
| | | GACGGGGGCCCGCACAAGCGGTGGAGCATGTGGTTTAATTCGAAGCT |
| | | ACGCGAAGAACCTTACCAGGTCTTGACATCTTCTGCCAATCTTAGAGA |
| | | TAAGACGTTCCCTTCGGGGACAGAATGACAGGTGGTGCATGGTTGTC |
| | | GTCAGCTCGTGTCGTGAGATGTTGGGTTAAGTCCCGCAACGAGCGCA |
| | | ACCCTTATTATCAGTTGCCAGCATTCAGTTGGGCACTCTGGTGAGACT |
| | | GCCGGTGACAAACCGGAGGAAGGTGGGGATGACGTCAAATCATCATG |
| | | CCCCTTATGACCTGGGCTACACACGTGCTACAATGGACGGTACAACG |
| | | AGTTGCGAAGTCGTGAGGCTAAGCTAATCTCTTAAAGCCGTTCTCAGT |
| | | TCGGATTGTAGGCTGCAACTCGCCTACATGAAGTTGGAATCGCTAGTA |
| | | ATCGCGGATCAGCATGCCGCGGTGAATACGTTCCCGGGCCTTGTACA |
| | | CACCGCCCGTCACACCATGAGAGTTTGTAACACCCAAAGCCGGTGAG |
| | | ATAACCTTCGGGAGTCAGCCGTCTAAGGTGGGACAGATGATTAGGGT |
| | | GAAGTCGTAACAAGGTAGCCGTAGGAGAACCTGCGGCTGGATCACCT |
| | | CCTT |
| 159 | DP95 16S rRNA | TGCTAATACCGCATAGATCCAAGAACCGCATGGTTCTTGGCTGAAAG |
| | | ATGGCGTAAGCTATCGCTTTTGGATGGACCCGCGGCGTATTAGCTAGT |
| | | TGGTGAGGTAATGGCTCACCAAGGCGATGATACGTAGCCGAACTGAG |
| | | AGGTTGATCGGCCACATTGGGACTGAGACACGGCCCAAACTCCTACG |
| | | GGAGGCAGCAGTAGGGAATCTTCCACAATGGACGCAAGTCTGATGGA |
| | | GCAACGCCGCGTGAGTGAAGAAGGCTTTCGGGTCGTAAAACTCTGTT |
| | | GTTGGAGAAGAATGGTCGGCAGAGTAACTGTTGTCGGCGTGACGGTA |
| | | TCCAACCAGAAAGCCACGGCTAACTACGTGCCAGCAGCCGCGGTAAT |
| | | ACGTAGGTGGCAAGCGTTATCCGGATTTATTGGGCGTAAAGCGAGCG |
| | | CAGGCGGTTTTTTAAGTCTGATGTGAAAGCCCTCGGCTTAACCGAGGA |
| | | AGCGCATCGGAAACTGGGAAACTTGAGTGCAGAAGAGGACAGTGGA |
| | | ACTCCATGTGTAGCGGTGAAATGCGTAGATATATGGAAGAACACCAG |
| | | TGGCGAAGGCGGCTGTCTGGTCTGTAACTGACGCTGAGGCTCGAAAG |
| | | CATGGGTAGCGAACAGGATTAGATACCCTGGTAGTCCATGCCGTAAA |
| | | CGATGAATGCTAGGTGTTGGAGGGTTTCCGCCCTTCAGTGCCGCAGCT |
| | | AACGCATTAAGCATTCCGCCTGGGGAGTACGACCGCAAGGTTGAAAC |
| | | TCAAAGGAATTGACGGGGGCCCGCACAAGCGGTGGAGCATGTGGTTT |
| | | AATTCGAAGCAACGCGAAGAACCTTACCAGGTCTTGACATCTTTTGAT |
| | | CACCTGAGAGATCAGGTTTCCCCTTCGGGGGCAAAATGACAGGTGGT |
| | | GCATGGTTGTCGTCAGCTCGTGTCGTGAGATGTTGGGTTAAGTCCCGC |
| | | AACGAGCGCAACCCTTATGACTAGTTGCCAGCATTTAGTTGGGCACTC |
| | | TAGTAAGACTGCCGGTGACAAACCGGAGGAAGGTGGGGATGACGTC |
| | | AAATCATCATGCCCCTTATGACCTGGGCTACACACGTGCTACAATGGA |
| | | TGGTACAACGAGTTGCGAGACCGCGAGGTCAAGCTAATCTCTTAAAG |
| | | CCATTCTCAGTTCGGACTGTAGGCTGCAACTCGCCTACACGAAGTCGG |
| | | AATCGCTAGTAATCGCGGATCAGCACGCCGCGGTGAATACGTTCCCG |
| | | GGCCTTGTACACACCGCCCGTCACACCATGAGAGTTTGTAACACCCG |
| | | AAGCCGGTGGCGTAACCCTTTTAGGGAGCGAGCCGTCTAAGGTGGGA |
| | | CAAATGATTAGGGTGAAGTCGTAACAAGGTAGCCGTAGGAGAACCTG |
| | | CGGCTGGATCACCTCCTTT |
| 160 | DP96 16S rRNA | ACACGGCCCAAACTCCTACGGGAGGCAGCAGTAGGGAATCTTCCACA |
| | | ATGGACGCAAGTCTGATGGAGCAACGCCGCGTGAGTGAAGAAGGCTT |
| | | TCGGGTCGTAAAACTCTGTTGTTGGAGAAGAATGGTCGGCAGAGTAA |
| | | CTGTTGTCGGCGTGACGGTATCCAACCAGAAAGCCACGGCTAACTAC |
| | | GTGCCAGCAGCCGCGGTAATACGTAGGTGGCAAGCGTTATCCGGATT |
| | | TATTGGGCGTAAAGCGAGCGCAGGCGGTTTTTTAAGTCTGATGTGAA |
| | | AGCCCTCGGCTTAACCGAGGAAGCGCATCGGAAACTGGGAAACTTGA |
| | | GTGCAGAAGAGGACAGTGGAACTCCATGTGTAGCGGTGAAATGCGTA |
| | | GATATATGGAAGAACACCAGTGGCGAAGGCGGCTGTCTGGTCTGTAA |
| | | CTGACGCTGAGGCTCGAAAGCATGGGTAGCGAACAGGATTAGATACC |
| | | CTGGTAGTCCATGCCGTAAACGATGAATGCTAGGTGTTGGAGGGTTTC |
| | | CGCCCTTCAGTGCCGCAGCTAACGCATTAAGCATTCCGCCTGGGGAGT |
| | | ACGACCGCAAGGTTGAAACTCAAAGGAATTGACGGGGGCCCGCACA |
| | | AGCGGTGGAGCATGTGGTTTAATTCGAAGCAACGCGAAGAACCTTAC |
| | | CAGGTCTTGACATCTTTTGATCACCTGAGAGATCAGGTTTCCCCTTCG |
| | | GGGGCAAAATGACAGGTGGTGCATGGTTGTCGTCAGCTCGTGTCGTG |
| | | AGATGTTGGGTTAAGTCCCGCAACGAGCGCAACCCTTATGACTAGTT |
| | | GCCAGCATTTAGTTGGGCACTCTAGTAAGACTGCCGGTGACAAACCG |
| | | GAGGAAGGTGGGGATGACGTCAAATCATCATGCCCCTTATGACCTGG |
| | | GCTACACACGTGCTACAATGGATGGTACAACGAGTTGCGAGACCGCG |
| | | AGGTCAAGCTAATCTCTTAAAGCCATTCTCAGTTCGGACTGTAGGCTG |
| | | CAACTCGCCTACACGAAGTCGGAATCGCTAGTAATCGCGGATCAGCA |
| | | CGCCGCGGTGAATACGTTCCCGGGCCTTGTACACACCGCCCGTCACAC |
| | | CATGAGAGTTTGTAACACCCGAAGCCGGTGGCGTAACCCTTTTAGGG |
| | | AGCGAGCCGTCTAAGGTGGGACAAATGATTAGGGTGAAGTCGTAACA |
| | | AGGTAGCCGTAGGAGAACCTGCGGCTGGATCACCTCCTTT |
| 161 | DP97 16S rRNA | AATGAGAGTTTGATCCTGGCTCAGGACGAACGCTGGCGGCGTGCCTA |
| | | ATACATGCAAGTCGAGCGATGATTAAAGATAGCTTGCTATTTTTATGA |
| | | AGAGCGGCGAACGGGTGAGTAACGCGTGGGAAATCTGCCGAGTAGC |
| | | GGGGGACAACGTTTGGAAACGAACGCTAATACCGCATAACAATGAGA |
| | | ATCGCATGATTCTTATTTAAAAGAAGCAATTGCTTCACTACTTGATGA |
| | | TCCCGCGTTGTATTAGCTAGTTGGTAGTGTAAAGGACTACCAAGGCG |
| | | ATGATACATAGCCGACCTGAGAGGGTGATCGGCCACACTGGGACTGA |
| | | GACACGGCCCAGACTCCTACGGGAGGCAGCAGTAGGGAATCTTCGGC |
| | | AATGGGGGCAACCCTGACCGAGCAACGCCGCGTGAGTGAAGAAGGTT |
| | | TTCGGATCGTAAAACTCTGTTGTTAGAGAAGAACGTTAAGTAGAGTG |
| | | GAAAATTACTTAAGTGACGGTATCTAACCAGAAAGGGACGGCTAACT |
| | | ACGTGCCAGCAGCCGCGGTAATACGTAGGTCCCAAGCGTTGTCCGGA |
| | | TTTATTGGGCGTAAAGCGAGCGCAGGTGGTTTCTTAAGTCTGATGTAA |
| | | AAGGCAGTGGCTCAACCATTGTGTGCATTGGAAACTGGGAGACTTGA |
| | | GTGCAGGAGAGGAGAGTGGAATTCCATGTGTAGCGGTGAAATGCGTA |
| | | GATATATGGAGGAACACCGGAGGCGAAAGCGGCTCTCTGGCCTGTAA |
| | | CTGACACTGAGGCTCGAAAGCGTGGGGAGCAAACAGGATTAGATACC |
| | | CTGGTAGTCCACGCCGTAAACGATGAGTGCTAGCTGTAGGGAGCTAT |
| | | AAGTTCTCTGTAGCGCAGCTAACGCATTAAGCACTCCGCCTGGGGAG |
| | | TACGACCGCAAGGTTGAAACTCAAAGGAATTGACGGGGGCCCGCACA |
| | | AGCGGTGGAGCATGTGGTTTAATTCGAAGCAACGCGAAGAACCTTAC |
| | | CAGGTCTTGACATACTCGTGATATCCTTAGAGATAAGGAGTTCCTTCG |
| | | GGACACGGGATACAGGTGGTGCATGGTTGTCGTCAGCTCGTGTCGTG |
| | | AGATGTTGGGTTAAGTCCCGCAACGAGCGCAACCCTTATTACTAGTTG |
| | | CCATCATTAAGTTGGGCACTCTAGTGAGACTGCCGGTGATAAACCGG |
| | | AGGAAGGTGGGGATGACGTCAAATCATCATGCCCCTTATGACCTGGG |
| | | CTACACACGTGCTACAATGGATGGTACAACGAGTCGCCAACCCGCGA |
| | | GGGTGCGCTAATCTCTTAAAACCATTCTCAGTTCGGATTGCAGGCTGC |
| | | AACTCGCCTGCATGAAGTCGGAATCGCTAGTAATCGCGGATCAGCAC |
| | | GCCGCGGTGAATACGTTCCCGGGCCTTGTACACACCGCCCGTCACACC |
| | | ACGGAAGTTGGGAGTACCCAAAGTAGGTTGCCTAACCGCAAGGAGGG |
| | | CGCTTCCTAAGGTAAGACCGATGACTGGGGTGAAGTCGTAACAAGGT |
| | | AGCCGTATCGGAAGGTGCGGCTGGATCACCTCCTTT |
| 162 | DP98 16S rRNA | AATGAGAGTTTGATCCTGGCTCAGGACGAACGCTGGCGGCGTGCCTA |
| | | ATACATGCAAGTCGAGCGATGATTAAAGATAGCTTGCTATTTTTATGA |
| | | AGAGCGGCGAACGGGTGAGTAACGCGTGGGAAATCTGCCGAGTAGC |
| | | GGGGGACAACGTTTGGAAACGAACGCTAATACCGCATAACAATGAGA |
| | | ATCGCATGATTCTTATTTAAAAGAAGCAATTGCTTCACTACTTGATGA |
| | | TCCCGCGTTGTATTAGCTAGTTGGTAGTGTAAAGGACTACCAAGGCG |
| | | ATGATACATAGCCGACCTGAGAGGGTGATCGGCCACACTGGGACTGA |
| | | GACACGGCCCAGACTCCTACGGGAGGCAGCAGTAGGGAATCTTCGGC |
| | | AATGGGGGCAACCCTGACCGAGCAACGCCGCGTGAGTGAAGAAGGTT |
| | | TTCGGATCGTAAAACTCTGTTGTTAGAGAAGAACGTTAAGTAGAGTG |
| | | GAAAATTACTTAAGTGACGGTATCTAACCAGAAAGGGACGGCTAACT |
| | | ACGTGCCAGCAGCCGCGGTAATACGTAGGTCCCAAGCGTTGTCCGGA |
| | | TTTATTGGGCGTAAAGCGAGCGCAGGTGGTTTCTTAAGTCTGATGTAA |
| | | AAGGCAGTGGCTCAACCATTGTGTGCATTGGAAACTGGGAGACTTGA |
| | | GTGCAGGAGAGGAGAGTGGAATTCCATGTGTAGCGGTGAAATGCGTA |
| | | GATATATGGAGGAACACCGGAGGCGAAAGCGGCTCTCTGGCCTGTAA |
| | | CTGACACTGAGGCTCGAAAGCGTGGGGAGCAAACAGGATTAGATACC |
| | | CTGGTAGTCCACGCCGTAAACGATGAGTGCTAGCTGTAGGGAGCTAT |
| | | AAGTTCTCTGTAGCGCAGCTAACGCATTAAGCACTCCGCCTGGGGAG |
| | | TACGACCGCAAGGTTGAAACTCAAAGGAATTGACGGGGGCCCGCACA |
| | | AGCGGTGGAGCATGTGGTTTAATTCGAAGCAACGCGAAGAACCTTAC |
| | | CAGGTCTTGACATACTCGTGATATCCTTAGAGATAAGGAGTTCCTTCG |
| | | GGACACGGGATACAGGTGGTGCATGGTTGTCGTCAGCTCGTGTCGTG |
| | | AGATGTTGGGTTAAGTCCCGCAACGAGCGCAACCCTTATTACTAGTTG |
| | | CCATCATTAAGTTGGGCACTCTAGTGAGACTGCCGGTGATAAACCGG |
| | | AGGAAGGTGGGGATGACGTCAAATCATCATGCCCCTTATGACCTGGG |
| | | CTACACACGTGCTACAATGGATGGTACAACGAGTCGCCAACCCGCGA |
| | | GGGTGCGCTAATCTCTTAAAACCATTCTCAGTTCGGATTGCAGGCTGC |
| | | AACTCGCCTGCATGAAGTCGGAATCGCTAGTAATCGCGGATCAGCAC |
| | | GCCGCGGTGAATACGTTCCCGGGCCTTGTACACACCGCCCGTCACACC |
| | | ACGGAAGTTGGGAGTACCCAAAGTAGGTTGCCTAACCGCAAGGAGGG |
| | | CGCTTCCTAAGGTAAGACCGATGACTGGGGTGAAGTCGTAACAAGGT |
| | | AGCCGTATCGGAAGGTGCGGCTGGATCACCTCCTTT |
| 163 | DP100 16S rRNA | TTTGAGAGTTTGATCCTGGCTCAGGACGAACGCTGGCGGCGTGCCTA |
| | | ATACATGCAAGTCGAACGAACTCTGGTATTGATTGGTGCTTGCATCAT |
| | | GATTTACATTTGAGTGAGTGGCGAACTGGTGAGTAACACGTGGGAAA |
| | | CCTGCCCAGAAGCGGGGGATAACACCTGGAAACAGATGCTAATACCG |
| | | CATAACAACTTGGACCGCATGGTCCGAGCTTGAAAGATGGCTTCGGC |
| | | TATCACTTTTGGATGGTCCCGCGGCGTATTAGCTAGATGGTGGGGTAA |
| | | CGGCTCACCATGGCAATGATACGTAGCCGACCTGAGAGGGTAATCGG |
| | | CCACATTGGGACTGAGACACGGCCCAAACTCCTACGGGAGGCAGCAG |
| | | TAGGGAATCTTCCACAATGGACGAAAGTCTGATGGAGCAACGCCGCG |
| | | TGAGTGAAGAAGGGTTTCGGCTCGTAAAACTCTGTTGTTAAAGAAGA |
| | | ACATATCTGAGAGTAACTGTTCAGGTATTGACGGTATTTAACCAGAA |
| | | AGCCACGGCTAACTACGTGCCAGCAGCCGCGGTAATACGTAGGTGGC |
| | | AAGCGTTGTCCGGATTTATTGGGCGTAAAGCGAGCGCAGGCGGTTTTT |
| | | TAAGTCTGATGTGAAAGCCTTCGGCTCAACCGAAGAAGTGCATCGGA |
| | | AACTGGGAAACTTGAGTGCAGAAGAGGACAGTGGAACTCCATGTGTA |
| | | GCGGTGAAATGCGTAGATATATGGAAGAACACCAGTGGCGAAGGCG |
| | | GCTGTCTGGTCTGTAACTGACGCTGAGGCTCGAAAGTATGGGTAGCA |
| | | AACAGGATTAGATACCCTGGTAGTCCATACCGTAAACGATGAATGCT |
| | | AAGTGTTGGAGGGTTTCCGCCCTTCAGTGCTGCAGCTAACGCATTAAG |
| | | CATTCCGCCTGGGGAGTACGGCCGCAAGGCTGAAACTCAAAGGAATT |
| | | GACGGGGGCCCGCACAAGCGGTGGAGCATGTGGTTTAATTCGAAGCT |
| | | ACGCGAAGAACCTTACCAGGTCTTGACATACTATGCAAATCTAAGAG |
| | | ATTAGACGTTCCCTTCGGGGACATGGATACAGGTGGTGCATGGTTGTC |
| | | GTCAGCTCGTGTCGTGAGATGTTGGGTTAAGTCCCGCAACGAGCGCA |
| | | ACCCTTATTATCAGTTGCCAGCATTAAGTTGGGCACTCTGGTGAGACT |
| | | GCCGGTGACAAACCGGAGGAAGGTGGGGATGACGTCAAATCATCATG |
| | | CCCCTTATGACCTGGGCTACACACGTGCTACAATGG |
| 164 | DP101 16S rRNA | ATGAGAGTTTGATCTTGGCTCAGGATGAACGCTGGCGGCGTGCCTAA |
| | | TACATGCAAGTCGAACGAACTTCCGTTAATTGATTATGACGTACTTGT |
| | | ACTGATTGAGATTTTAACACGAAGTGAGTGGCGAACGGGTGAGTAAC |
| | | ACGTGGGTAACCTGCCCAGAAGTAGGGGATAACACCTGGAAACAGAT |
| | | GCTAATACCGTATAACAGAGAAAACCGCATGGTTTTCTTTTAAAAGAT |
| | | GGCTCTGCTATCACTTCTGGATGGACCCGCGGCGTATTAGCTAGTTGG |
| | | TGAGGCAAAGGCTCACCAAGGCAGTGATACGTAGCCGACCTGAGAGG |
| | | GTAATCGGCCACATTGGGACTGAGACACGGCCCAGACTCCTACGGGA |
| | | GGCAGCAGTAGGGAATCTTCCACAATGGACGCAAGTCTGATGGAGCA |
| | | ACGCCGCGTGAGTGAAGAAGGGTTTCGGCTCGTAAAGCTCTGTTGTT |
| | | AAAGAAGAACGTGGGTAAGAGTAACTGTTTACCCAGTGACGGTATTT |
| | | AACCAGAAAGCCACGGCTAACTACGTGCCAGCAGCCGCGGTAATACG |
| | | TAGGTGGCAAGCGTTATCCGGATTTATTGGGCGTAAAGCGAGCGCAG |
| | | GCGGTCTTTTAAGTCTAATGTGAAAGCCTTCGGCTCAACCGAAGAAGT |
| | | GCATTGGAAACTGGGAGACTTGAGTGCAGAAGAGGACAGTGGAACTC |
| | | CATGTGTAGCGGTGAAATGCGTAGATATATGGAAGAACACCAGTGGC |
| | | GAAGGCGGCTGTCTGGTCTGCAACTGACGCTGAGGCTCGAAAGCATG |
| | | GGTAGCGAACAGGATTAGATACCCTGGTAGTCCATGCCGTAAACGAT |
| | | GATTACTAAGTGTTGGAGGGTTTCCGCCCTTCAGTGCTGCAGCTAACG |
| | | CATTAAGTAATCCGCCTGGGGAGTACGACCGCAAGGTTGAAACTCAA |
| | | AAGAATTGACGGGGGCCCGCACAAGCGGTGGAGCATGTGGTTTAATT |
| | | CGAAGCTACGCGAAGAACCTTACCAGGTCTTGACATCTTCTGACAGTC |
| | | TAAGAGATTAGAGGTTCCCTTCGGGGACAGAATGACAGGTGGTGCAT |
| | | GGTTGTCGTCAGCTCGTGTCGTGAGATGTTGGGTTAAGTCCCGCAACG |
| | | AGCGCAACCCTTATTACTAGTTGCCAGCATTAAGTTGGGCACTCTAGT |
| | | GAGACTGCCGGTGACAAACCGGAGGAAGGTGGGGACGACGTCAAAT |
| | | CATCATGCCCCTTATGACCTGGGCTACACACGTGCTACAATGGATGGT |
| | | ACAACGAGTCGCGAGACCGCGAGGTTAAGCTAATCTCTTAAAACCAT |
| | | TCTCAGTTCGGACTGTAGGCTGCAACTCGCCTACACGAAGTCGGAATC |
| | | GCTAGTAATCGCGGATCAGCATGCCGCGGTGAATACGTTCCCGGGCC |
| | | TTGTACACACCGCCCGTCACACCATGAGAGTTTGTAAC |
| 165 | DP101 ITS | TCCGTAGGTGAACCTGCGGAAGGATCATTACTGTGATTTAGTACTACA |
| | sequence | CTGCGTGAGCGGAACGAAAACAACAACACCTAAAATGTGGAATATAG |
| | | CATATAGTCGACAAGAGAAATCTACGAAAAACAAACAAAACTTTCAA |
| | | CAACGGATCTCTTGGTTCTCGCATCGATGAAGAGCGCAGCGAAATGC |
| | | GATACCTAGTGTGAATTGCAGCCATCGTGAATCATCGAGTTCTTGAAC |
| | | GCACATTGCGCCCCTCGGCATTCCGGGGGGCATGCCTGTTTGAGCGTC |
| | | GTTTCCATCTTGCGCGTGCGCAGAGTTGGGGGAGCGGAGCGGACGAC |
| | | GTGTAAAGAGCGTCGGAGCTGCGACTCGCCTGAAAGGGAGCGAAGCT |
| | | GGCCGAGCGAACTAGACTTTTTTTCAGGGACGCTTGGCGGCCGAGAG |
| | | CGAGTGTTGCGAGACAACAAAAAGCTCGACCTCAAATCAGGTAGGAA |
| | | TACCCGCTGAACTTAAGCATATCAATAAGCGGAGGAAAAGAAACCAA |
| | | CAGGGATTGCCTCAGTAGCGGCGAGTGAAGCGGCAAGAGCTCAGATT |
| | | TGAAATCGTGCTTTGCGGCACGAGTTGTAGATTGCAGGTTGGAGTCTG |
| | | TGTGGAAGGCGGTGTCCAAGTCCCTTGGAACAGGGCGCCCAGGAGGG |
| | | TGAGAGCCCCGTGGGATGCCGGCGGAAGCAGTGAGGCCCTTCTGACG |
| | | AGTCGAGTTGTTTGGGAATGCAGCTCCAAGCGGGTGGTAAATTCCAT |
| | | CTAAGGCTAAATACTGGCGAGAGACCGATAGCGAACAAGTACTGTGA |
| | | AGGAAAGATGAAAAGCACTTTGAAAAGAGAGTGAAACAGCACGTGA |
| | | AATTGTTGAAAGGGAAGGGTATTGCGCCCGACATGGGGATTGCGCAC |
| | | CGCTGCCTCTCGTGGGCGGCGCTCTGGGCTTTCCCTGGGCCAGCATCG |
| | | GTTCTTGCTGCAGGAGAAGGGGTTCTGGAACGTGGCTCTTCGGAGTGT |
| | | TATAGCCAGGGCCAGATGCTGCGTGCGGGGACCGAGGACTGCGGCCG |
| | | TGTAGGTCACGGATGCTGGCAGAACGGCGCAACACCGCCCGTCTTGA |
| | | AACATGGACCAAGGAGTCTAACGTCTATGCGAGTGTTTGGGTGTGAA |
| | | ACCCGTACGCGTAATGAAAGTGAACGTAGGTCGGACCCCCTGCCCTC |
| | | GGGGAGGGGAGCACGATCGACCGATCCCGATGTTTATCGGAAGGATT |
| | | TGAGTAGGAGCATAGCTGTTGGGACCCGAAAGATGGTGAACTATGCC |
| | | TGAATAGGGTGAAGCCAGAGGAAACTCTGGTGGAGGCTCGTAGCGGT |
| | | TCTGACGTGCAAATCGATCGTCGAATTTGGGTATAGGGGCGAAAGAC |
| | | TAATCGAACCATCTAGTAGCTGGTTCCTGCCGAAGTTTCCCTCAGGA |

1.-20. (canceled)

21. A dietary supplement comprising a combination of four heterologous microbes selected from Lactobacillus brevis, Lactobacillus plantarum, Leuconostoc mesenteroides, and a Pichia, wherein the combination of four heterologous microbes comprises at least 1×10\hat{ }7 of each of the four heterologous microbes, wherein each of the four heterologous microbes or the combination thereof comprises a gene or gene pathway directed to biosynthesis of short chain fatty acid (SCFA) or metabolites thereof, and wherein the combination produces an increased amount of SCFA when grown together relative to the summed amount of SCFA produced by an equivalent amount of each distinct microbe grown in isolation under the same conditions, wherein the combination lessens a decrease in, maintains, or improves gut health in a subject, and wherein the dietary supplement is formulated for oral delivery.

22. The dietary supplement of claim 21, wherein the combination lessens a decrease in, maintains, or improves gut barrier function in the subject.

23. The dietary supplement of claim 21, wherein the dietary supplement is formulated as a medical food or a pharmaceutical composition.

24. The dietary supplement of claim 21, wherein the combination comprises about 1.0×10⁷ to 1.0×10¹² CFU of each of the heterologous microbes.

25. The dietary supplement of claim 22, wherein, following administration of the dietary supplement to the subject over a period of time, gut barrier function in the subject is maintained or improved as compared to a suitable control.

26. The dietary supplement of claim 25, wherein the suitable control comprises (i) a control group that has not been administered the dietary supplement and/or (ii) the subject's gut barrier function prior to the first administration of the dietary supplement to the subject.

27. The dietary supplement of claim 22, wherein, following administration of the dietary supplement to the subject over a period of time: (i) the level of alkaline phosphatase (ALP) in the subject's intestine is maintained or improved as compared to a suitable control, and/or (ii) a decrease in intestinal ALP in the subject is less severe as compared to a suitable control.

28. The dietary supplement of claim 27, wherein the suitable control comprises (i) a control group that has not been administered the dietary supplement and/or (ii) the subject's intestinal alkaline phosphatase (ALP) prior to the first administration of the dietary supplement to the subject.

29. The dietary supplement of claim 21, wherein the taxonomic or functional composition of the microbiome of the subject is altered after administration of the dietary supplement to the subject, as compared to a suitable control.

30. The dietary supplement of claim 29, wherein the suitable control comprises (i) a control group that has not been administered the dietary supplement, and/or (ii) the taxonomic or functional composition of the microbiome of the subject prior to the first administration of the dietary supplement.

31. The dietary supplement of claim 29, wherein the microbiome is altered by an increase in the abundance of the microbial species present in the dietary supplement and/or wherein the microbiome is altered by increased gene abundance of vitamin K2 biosynthesis pathways.

32. The dietary supplement of claim 21, wherein administration of the dietary supplement to the subject results in (i) altering the amount of at least one circulatory inflammatory cytokine in the subject, wherein the amount of the at least one circulatory inflammatory cytokine is altered as compared to a suitable control, optionally wherein the suitable control comprises (a) a control group that has not been administered the dietary supplement, and/or (b) the amount of the at least one circulatory inflammatory cytokine in the subject prior to the first administration of the dietary supplement.

33. The dietary supplement of claim 32, wherein the at least one circulatory inflammatory cytokine is selected from the group consisting of CRP, IL-17, TNF, IL-4, RANKL, and IFNγ, optionally wherein the amount of the at least one circulatory inflammatory cytokine decreases.

34. A dietary supplement comprising a combination of four heterologous microbes consisting of Lactobacillus brevis, Lactobacillus plantarum, a Leuconostoc, and a Pichia, wherein the combination of four heterologous microbes comprises at least 1×10\hat{ }7 of each of the four heterologous microbes, wherein each of the four heterologous microbes comprises a gene or gene pathway directed to biosynthesis of short chain fatty acid (SCFA) or metabolites thereof and wherein the combination produces an increased amount of SCFA when grown together relative to the summed amount of SCFA produced by an equivalent amount of each distinct microbe grown in isolation under the same conditions, wherein the combination decreases systemic inflammation in a subject, and wherein the dietary supplement is formulated for oral delivery.

35. The dietary supplement of claim 34, wherein following administration of the dietary supplement to the subject over a period of time, the abundance of Bifidobacterium pseudolongum is increased in the subject's gut microbiome as compared to a suitable control.

36. The dietary supplement of claim 35, wherein the suitable control is (i) a control group that has not been administered the dietary supplement and/or (ii) the presence or severity of the subject's systemic inflammation prior to the first administration of the dietary supplement

37. The dietary supplement of claim 21, wherein the dietary supplement further comprises at least one of: (i) a prebiotic selected from an oligofructose or a dried fruit or vegetable powder; (ii) a bulking agent comprising magnesium stearate; and (iii) a cryoprotectant present in an effective amount to extend survival of the heterologous microbes after thawing the dietary supplement from a cryogenic temperature.

38. A composition comprising a combination of four heterologous microbes selected from Lactobacillus brevis, Lactobacillus plantarum, Leuconostoc mesenteroides, and a Pichia, wherein the combination of four heterologous microbes comprises at least 1×10\hat{ }7 of each of the four heterologous microbes, wherein the composition lessens a decrease in, maintains, or improves immune health in a subject, or reduces inflammation in the subject, and wherein the composition is formulated for oral delivery.

39. The composition of claim 38, wherein administration of the composition to the subject results in (i) altering the amount of at least one circulatory inflammatory cytokine in the subject, wherein the amount of the at least one circulatory inflammatory cytokine is altered as compared to a suitable control, optionally wherein the suitable control comprises (a) a control group that has not been administered the composition, and/or (b) the amount of the at least one circulatory inflammatory cytokine in the subject prior to the first administration of the composition.

40. The composition of claim 39, wherein the at least one circulatory inflammatory cytokine is selected from the group consisting of CRP, IL-17, TNF, IL-4, RANKL, and IFNγ, optionally wherein the amount of the at least one circulatory inflammatory cytokine decreases.
